(19) United States
(12) Patent Application Publication
(10) Pub. No.: US 2026/0167659 A1
(43) Pub. Date: Jun. 18, 2026

(54) STING AGONISTS

(71) Applicant: LIGACHEM BIOSCIENCES INC., Daejeon (KR)

(72) Inventors: Ho Young Song, Daejeon (KR); Sung Min KIM, Daejeon (KR); Yeong Hun Jung, Daejeon (KR); Kun Jung Lee, Daejeon (KR); Ji Soo Kim, Daejeon (KR); Kyung Eun Park, Daejeon (KR); Se Yeon Park, Daejeon (KR); Yun-Hee Park, Daejeon (KR); Nara Han, Daejeon (KR); So Yeon Lim, Daejeon (KR); Gun Young Jang, Daejeon (KR); Chul-Woong Chung, Daejeon (KR); Jihye Oh, Daejeon (KR); Hyun Joo Bae, Daejeon (KR); Sook Kyung Chang, Daejeon (KR); Young Cheol Lee, Daejon (KR)

(21) Appl. No.: 19/127,024

(22) PCT Filed: Nov. 8, 2023
(86) PCT No.: PCT/IB23/00670

§ 371 Date: May 2, 2025

(30) Foreign Application Priority Data
Nov. 8, 2022 (KR) 10-2022-0147897

(51) Int. Cl. C07H 15/26; A61K 31/7056; A61P 35/00

(52) U.S. Cl. CPC C07H 15/26; A61K 31/7056; A61P 35/00

(57) ABSTRACT

The disclosure relates to a compound represented by Formula 1, or a pharmaceutically acceptable salt thereof, as a stimulator of interferon genes (STING) agonist, pharmaceutical compositions comprising the same, and related methods of and uses in treatment or prevention of disease.

### CROSS-REFERENCE TO RELATED APPLICATIONS

This application claims the benefit of Korean Patent Application No. 10-2022-0147897, filed Nov. 8, 2022, which is incorporated by reference herein in its entirety.

### BACKGROUND

The stimulator of interferon genes (STING) is a low-molecular-weight protein currently attracting attention as a target for cancer therapies. STING is an adapter protein in the cGAS (cyclic GMP-AMP synthase)-STING pathway, which is a sensing pathway that induces activation of type I IFN and other inflammatory cytokines, triggering antiviral and antitumor immune responses (Chen, Q. et al. Regulation and function of the cGAS-STING pathway of cytosolic DNA sensing. Nat. Immunol. 2016, 17, 1142-1149; Woo, S. R. et al. STING-dependent cytosolic DNA sensing mediates innate immune recognition of immunogenic tumors. Immunity 2014, 41, 830-842). In addition, STING activates signal transducer and activator of transcription 6 (STAT6) and transcription factor interferon regulatory factor 3 (IRF3) through TANK-binding kinase 1 (TBK1) in antiviral and innate immune responses (Burdette DL, Vance RE, STING and the innate immune response to nucleic acids in the cytosol, 2013, Nature Immunology. 14 (1): 19-26). STING agonists can also trigger expression of cytokines, giving rise to a T cell-mediated innate immune response which inhibits the growth of cancer cells. However, systemic delivery of STING agonists can cause widespread inflammation.

Various STING agonists have been tested in preclinical and clinical environments. A variety of agonists in the form of CDN (cyclic dinucleotide) compounds (ADU-S100, BI-STING, GSK532, JNJ-4412, SB11285, MK-1454, TAK676, etc.), bacterial vectors (SYNB1891, STACT-TREX-1), non-cyclic dinucleotide (CDN), compounds (ALG-031048, JNJ-6196, MK-2118, MSA-1, MSA-2, CRD-5500, etc.), nano vaccines (PC7A NP, cGAMP-NP, etc.) and ADCs (XMT-2056, TAK500, etc.) are under development by various strategies.

The most widely used preclinical compound, DMXAA (vascular disrupting agent), was used clinically in combination with paclitaxel and carboplatin, but its lack of efficacy was confirmed in phase 3. Further, ADU-S100, which was first used clinically as the STING agonist, was discontinued from use in 2020.

Examples of STING agonists are disclosed, for example, in WO2021/014365 (a macrocyclic compound as STING agonist), and US2021/0139473 (a heterocyclic amide-containing compound as protein modulator), US 2022/0073509 (a heterocyclic compound as STING activator), and KR 2022-0024467 (a heterocycle-containing STING agonist), each of which is incorporated herein by reference in its entirety.

However, existing STING agonists appear to exhibit only limited bioavailability and require local administration to tumors due to hyperactivation of cytokine expression, or have to be used in combination with other compounds. Therefore, there remains a demand for development of therapeutically effective STING agonists.

### SUMMARY

In certain aspects, the present disclosure relates to compounds represented by structural formula (I), and pharmaceutically acceptable salts thereof:

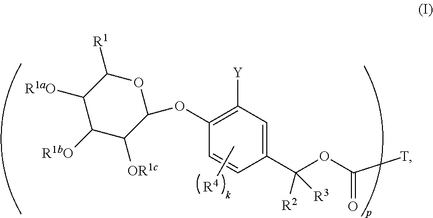

wherein:
T is a moiety comprising a Stimulator of Interferon Genes (STING) agonist,
p is 1 or 2,
each instance of R¹ is independently CH₂OR¹¹ or COOR¹²,
each instance of R^{1a}, R^{1b}, R^{1c} and R¹¹ is independently H or a hydroxyl protecting group,
each instance of R¹² is independently H or a carboxyl protecting group,
each instance of R² and R³ is independently H or alkyl, or R² and R³ together with a carbon atom to which they are attached form a cycloalkyl,
each instance of R⁴ is independently selected from halogen, alkyl, CN, and NO₂,
each instance of k is independently 0, 1, 2, or 3,
each instance of Y is independently selected from H, —C(O)NHL^{u}U, C(O)NR′(L^{u}U), —C(O)N(L^{u}U)₂, and —C(O)OH,
each instance of L^{u} is a first linker,
   each instance of U is independently selected from H, alkyl, amino, azido, acetylenyl, alkylamino, heterocyclyl, alkoxy, —COOH, —P(O)(OH)₂, —OH, -DBCO, and a saccharide, and
   each instance R′ is independently selected from alkyl, cycloalkyl, alkoxy, alkylthio, mono- or di-alkylamino, heteroaryl, and aryl.

In some aspects, the present disclosure relates to pharmaceutical compositions comprising a compound of the disclosure, such as a compound of formula (I), or a pharmaceutically acceptable salt thereof, and a pharmaceutically acceptable excipient.

In some aspects, the present disclosure relates to a method of preventing or treating a disease mediated by stimulator of interferon genes (STING) in a subject in need thereof, comprising administering to the subject a compound of the disclosure, such as a compound of formula (I), or a pharmaceutically acceptable salt thereof, or a pharmaceutical composition of the disclosure.

In some aspects, the present disclosure relates to a method of inducing an immune response in a subject in need thereof, comprising administering to the subject a compound of the disclosure, such as a compound of formula (I), or a pharmaceutically acceptable salt thereof, or a pharmaceutical composition of the disclosure.

In some aspects, the present disclosure relates to a method of modulating the activity of a STING adaptor protein comprising contacting the STING adaptor protein with a compound of the disclosure, such as a compound of formula (I), or a pharmaceutically acceptable salt thereof, or a pharmaceutical composition of the disclosure.

### BRIEF DESCRIPTION OF THE DRAWINGS

FIG. 1A: STING agonist compounds (Compound 214, 221 and 230) induced cytokine production (human CXCL-10).

FIG. 1B: STING agonist compounds (Compound 214, 221 and 230) induced cytokine production (human IFNα).

FIG. 2A: MDA-MB-468 tumor cells were treated with STING agonist compound (Compound 313).

FIG. 2B: BxPC3 tumor cells were treated with STING agonist compound (Compound 313).

FIG. 3A: Up-regulation of co-stimulatory molecule CD86 when THP-1 was treated with the STING agonist compound (Compound 313).

FIG. 3B: Up-regulation of MHC class II molecule HLA-DR when THP-1 was treated with the STING agonist compound (Compound 313).

FIG. 4A: STING agonist compounds (Compound 274 and 277) enhanced CD69 expression on CD8⁺ T cells.

FIG. 4B: STING agonist compounds (Compound 274 and 277) enhanced CD69 expression on NK cells.

FIG. 5A: STING agonist compounds (Compound 344 and 361) enhanced expansion of activated CD8⁺ T cells FIG. 5B: STING agonist compounds (Compound 344 and 361) enhanced expansion of activated NK cells.

FIG. 6A: Graphical representation of the plasma PK in naïve Balb/C mouse following single dose of STING agonist compound (Compound 221).

FIG. 6B: Graphical representation of the plasma PK in naïve Balb/C mouse following single dose of STING agonist compound (Compound 277).

FIG. 6C: Graphical representation of the plasma PK in naïve Balb/C mouse following single dose of STING agonist compound (Compound 281).

FIG. 6D: Graphical representation of the plasma PK in naïve Balb/C mouse following single dose of STING agonist compound (Compound 274) compared to Comparative compound #1 and #2.

FIG. 7A: Tumor volume (mm³) following treatment with STING agonist compounds (Compound 214 and 221) in CT26 syngeneic mouse model. When tumor volume reached 70 mm³, each compound was given at 1.5 mg/kg intravenously every 3 or 4 days (total three times).

FIG. 7B: Body weight (%) following treatment with STING agonist compounds (Compound 214 and 221). When tumor volume reached 70 mm³, each compound was given at 1.5 mg/kg intravenously every 3 or 4 days (total three times).

FIG. 8: Tumor volume (mm³) following treatment with STING agonist compound (Compound 274) in CT26 mouse model at various doses.

FIG. 9: Tumor volume (mm³) following treatment with STING agonist compounds (Compound 274 and 281) compared to Comparative compound #1 in CT26 syngeneic mouse model. When tumor volume reached 55 mm³, each compound was given at 0.5 mg/kg intravenously once weekly for three weeks. Compared to the comparative compound #1, mean tumor growth over time was monitored.

FIG. 10A: Tumor volume (mm³) following treatment with STING agonist compounds (Compound 274, 313, 344, and 396) compared to Comparative compound #3 in CT26 syngeneic mouse model. When tumor volume reached 80 mm³, each compound was given at 0.3 mg/kg intravenously once weekly for 3 weeks. Compared to the comparative compound #3, tumor growth over time was monitored.

FIG. 10B: Body weight (mm³) following treatment with STING agonist compounds (Compound 274, 313, 344, and 396) compared to Comparative compound #3 in CT26 syngeneic mouse model. When tumor volume reached 80 mm³, each compound was given at 0.3 mg/kg intravenously once weekly for 3 weeks. Compared to the comparative compound #3, body weight over time was monitored.

FIG. 11: Tumor volume (mm³) following treatment with STING agonist compounds (Compound 274, and 313) compared to Comparative compound #1 and #2 in EMT6 syngeneic mouse model. When tumor volume reached 100 mm³, each compound was given at 0.125 mg/kg intravenously once weekly for three weeks. Compared to the comparative compounds #1 and #2, mean tumor growth over time was monitored.

FIG. 12A: Tumor volume (mm³) following treatment with STING agonist compound (Compound 313) at various doses in EMT6 model.

FIG. 12B: Body weight (%) following treatment with STING agonist compound (Compound 313) at various doses in EMT6 model.

### DETAILED DESCRIPTION

In certain aspects, the present disclosure relates to compounds represented by structural formula (I), and pharmaceutically acceptable salts thereof:

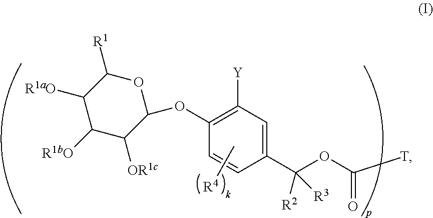

wherein:
T is a moiety comprising a Stimulator of Interferon Genes (STING) agonist,
p is 1 or 2,
each instance of R¹ is independently CH₂OR¹¹ or COOR¹²,
each instance of R^{1a}, R^{1b}, R^{1c}, and R¹¹ is independently H or a hydroxyl protecting group,
each instance of R¹² is independently H or a carboxyl protecting group,
each instance of R² and R³ is independently H or alkyl, or R² and R³ together with a carbon atom to which they are attached form a cycloalkyl,
each instance of R⁴ is independently selected from halogen, alkyl, CN, and NO₂,
each instance of k is independently 0, 1, 2, or 3,
each instance of Y is independently selected from H, —C(O)NHL^{u}U, C(O)NR′(L^{u}U), —C(O)N(L^{u}U)₂, and —C(O)OH,
each instance of L^{u} is a first linker,
each instance of U is independently selected from H, alkyl, amino, azido, acetylenyl, alkylamino, heterocyclyl, alkoxy, —COOH, —P(O)(OH)₂, —OH, -DBCO, and a saccharide, and
each instance R′ is independently selected from alkyl, cycloalkyl, alkoxy, alkylthio, mono- or di-alkylamino, heteroaryl, and aryl.

In some embodiments, the compound of formula (I) is a compound of formula (Ia):

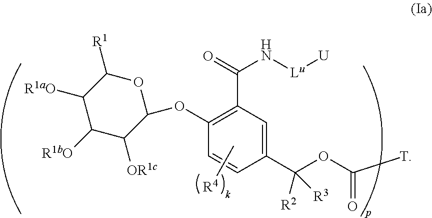

In some embodiments, p is 1 and T is a moiety represented by one of the following structural formulas:

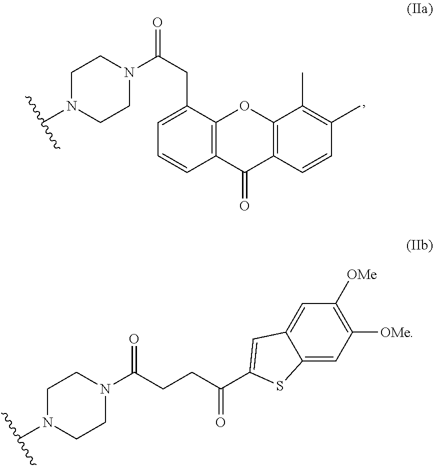 [SMILES: *N1CCN(C(=O)Cc2cccc3c(=O)c4ccc(C)c(C)c4oc23)CC1 ; *N1CCN(C(=O)CCC(=O)c2cc3cc(OC)c(OC)cc3s2)CC1]

In some embodiments, T is a moiety represented by formula (IIc):

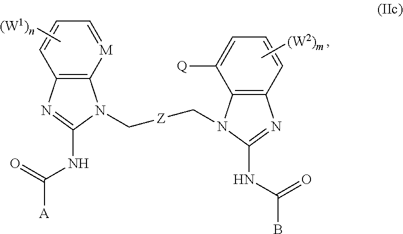

wherein:
T is coupled to the —C(O)OCR²R³— fragment of formula (I) via W¹, W², A, B, or L²,
M is N, C(X^{a}R^{a}) or C(X^{b}L¹L²),
Q is —X^{a}R^{a} or -XbL¹L²-,
each instance of W¹ and W² is independently selected from alkyl, amino, amido, carboxylic acid, ester, and hydrazido, e.g. —C(O)NH-alkyl-N(alkyl)-, or —C(O)NH—N(alkyl)-;
n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino, and —O(alkylene)guanidino,
L¹ is selected from alkylene, heteroalkylene, alkenylene, alkynylene, cycloalkylene, heterocyclylene, arylene, and heteroarylene,
L² is a bond, or a linker moiety coupled to L¹ and comprising a nitrogen atom coupled to the —C(O)O(CR²R³)— fragment of the compound represented by structural formula (I). Any suitable linker moiety may be used.

In some embodiments, Q is —X^{a}R^{a}.

In some embodiments, M is C(X^{a}R^{a}).

In some embodiments, Q is —X^{a}R^{a} or M is C(X^{a}R^{a}).

In some embodiments, X^{a} is O.

In some embodiments, R^{a} is selected from C₁₋₆ alkyl, heterocyclylalkyl, cycloalkylalkyl, -(alkylene)carboxylic acid, and -(alkylene) guanidino.

In some embodiments, the alkyl is C₁₋₆ alkyl, e.g., C₃ alkyl.

In some embodiments, the alkylene is C₁₋₆ alkylene, e.g., C₃ alkylene.

In some embodiments, M is C(X^{a}R^{a}), e.g., M has the structure selected from:

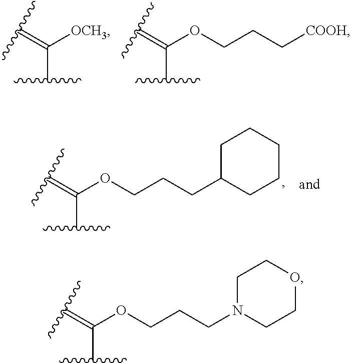 [SMILES: *C(=*)OC ; *C(=*)OCCCC(=O)O ; *C(=*)OCCCC1CCCCC1 ; *C(=*)OCCCN1CCOCC1]

wherein each

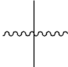

is a point of connection to the remainder of the phenyl ring.

In some embodiments, Q is —X^{a}R^{a}, e.g., Q has the structure selected from:

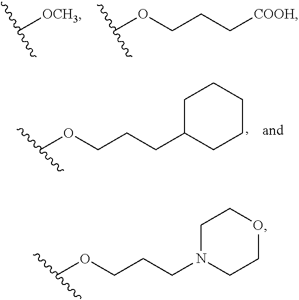

wherein each

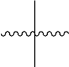

is a point of connection to the phenyl ring.

In some embodiments, M has the structure:

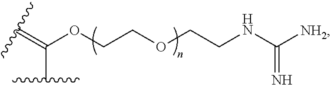

wherein n represents an integer from 1 to 15, e.g.,

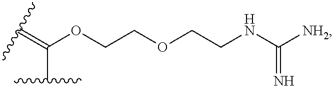 [SMILES: *C(=*)OCCOCCNC(=N)N]

wherein each

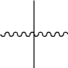

is a point of connection to the remainder of the phenyl ring.

In some embodiments, Q has the structure:

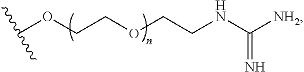

wherein n represents an integer from 1 to 15, e.g.,

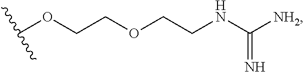 [SMILES: *OCCOCCNC(=N)N]

wherein

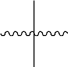

is a point of connection to the phenyl ring.

In some embodiments, M is C(X^{b}L¹L²-) or Q is —X^{b}L¹L²-.

In some embodiments, L² is selected from a bond,

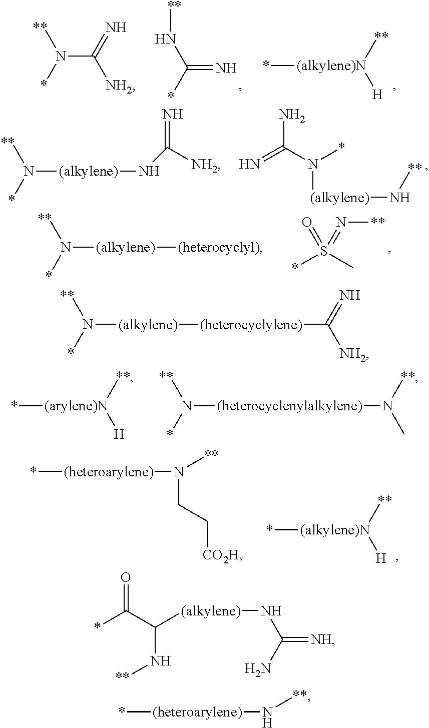

and *heterocyclylene** and wherein * is the point of connection to L¹ and ** is the point of connection to the —C(O)O(CR²R³)— fragment of the compound represented by structural formula (I).

In some embodiments, T is represented by structural formula (IIa).

In some embodiments, the compound is

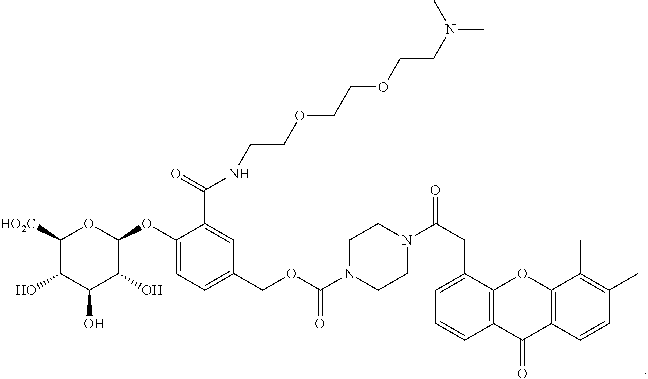 [SMILES: Cc1ccc2c(=O)c3cccc(CC(=O)N4CCN(C(=O)OCc5ccc(O[C@@H]6O[C@H](C(=O)O)[C@@H](O)[C@H](O)[C@H]6O)c(C(=O)NCCOCCOCCN(C)C)c5)CC4)c3oc2c1C]

In some embodiments, T is represented by structural formula (IIb).

In some embodiments, the compound is

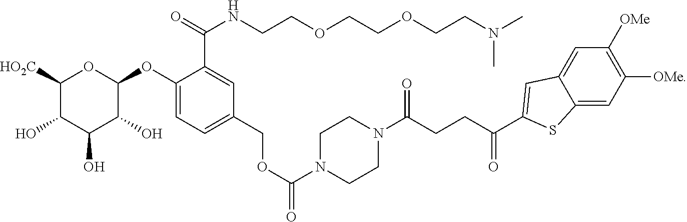 [SMILES: COc1cc2cc(C(=O)CCC(=O)N3CCN(C(=O)OCc4ccc(O[C@@H]5O[C@H](C(=O)O)[C@@H](O)[C@H](O)[C@H]5O)c(C(=O)NCCOCCOCCN(C)C)c4)CC3)sc2cc1OC]

In some embodiments, T is represented by structural formula (IIc).

In some embodiments, T is coupled to the —C(O)OCR²R³— fragment via W², and T is a moiety represented by formula (IIc1):

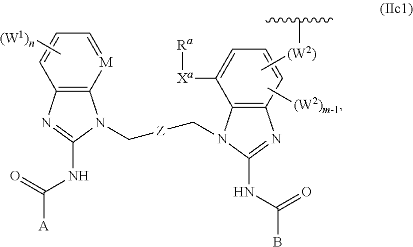

wherein

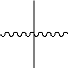

is the point of connection to the —C(O)O(CR²R³)— fragment of the compound represented by structural formula (I).

In some embodiments, T is coupled to the —C(O)OCR²R³— fragment via B, and T is a moiety represented by formula (IIc2):

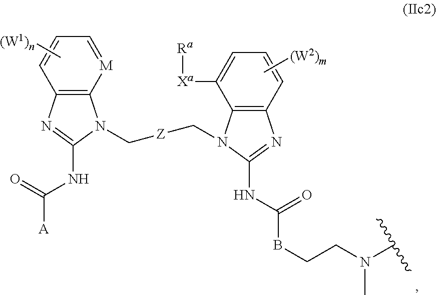

wherein

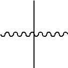

is the point of connection to the —C(O)O(CR²R³)— fragment of the compound represented by structural formula (I).

In some embodiments, T is coupled to the —C(O)OCR²R³— fragment via L², and T is a moiety represented by formula (IIc3):

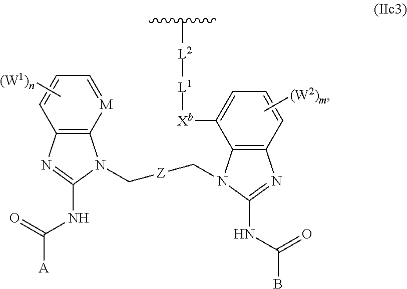

wherein

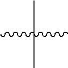

is the point of connection to the —C(O)O(CR²R³)— fragment of the compound represented by structural formula (I).

In some embodiments, T is coupled to the —C(O)OCR²R³— fragment via L², and T is a moiety represented by formula (IIc4):

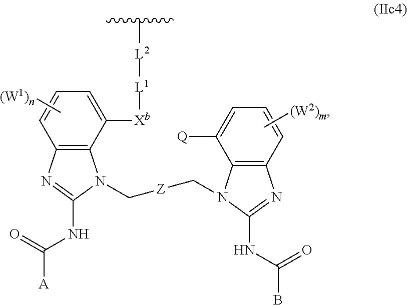

wherein

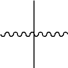

is the point of connection to the —C(O)O(CR²R³)— fragment of the compound represented by structural formula (I).

In some embodiments, L² is a second linker. Any suitable linker moiety may be used.

In some embodiments, L² is selected from a bond,

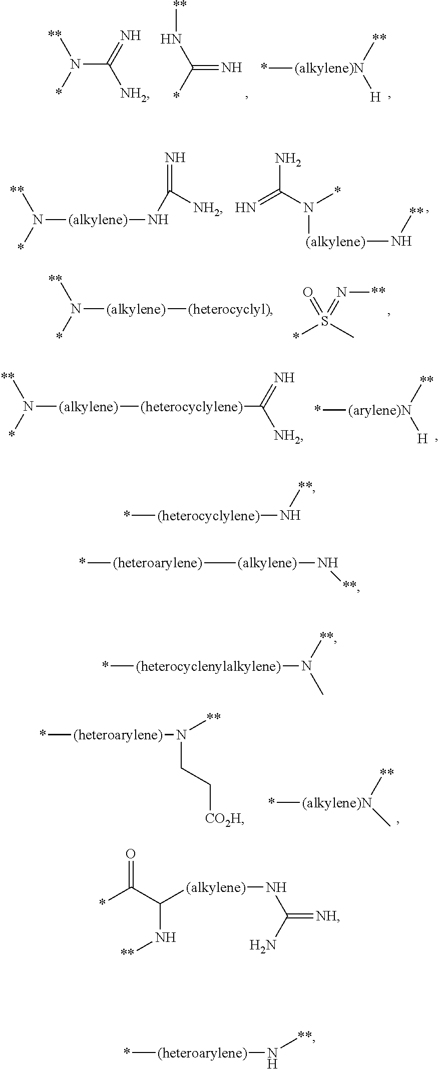

and *heterocyclylene**, and wherein * is the point of connection to L¹ and ** is the point of connection to the —C(O)O(CR²R³)— fragment of the compound represented by structural formula (I).

In some embodiments, L² is a second linker comprising ^{#}OC(O)NR-L⁴-NR⁶, ^{#}OC(O)-L⁴-NR⁶, or ^{#}OC(O)NR⁵-L⁴-(heterocyclylene), wherein:
the heterocyclylene comprises a nitrogen atom connected to the —C(O)O(CR²R³)— fragment of the compound represented by structural formula (I), and wherein # is the point of connection to L¹,
each instance of L⁴ is independently alkylene or arylalkylene,
each instance of R⁵ is independently selected from H, alkyl, and dialkylaminoalkyl, and
each instance of R⁶ is independently selected from H, alkyl, and dialkylaminoalkyl.

In some embodiments, L² is selected from

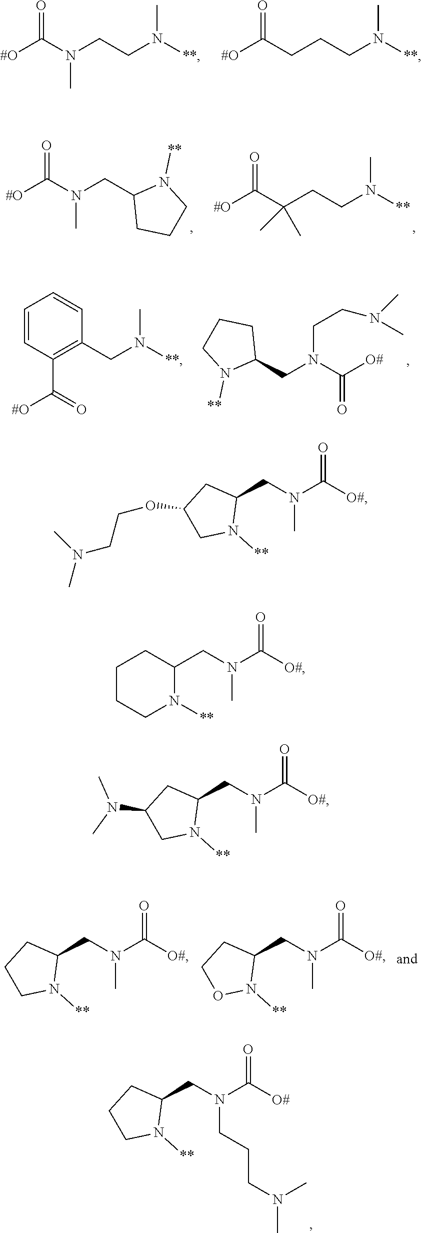

wherein ** indicates the connection point to the —C(O)O(CR²R³)— fragment of the compound represented by structural formula (I).

In some embodiments, L² is selected from

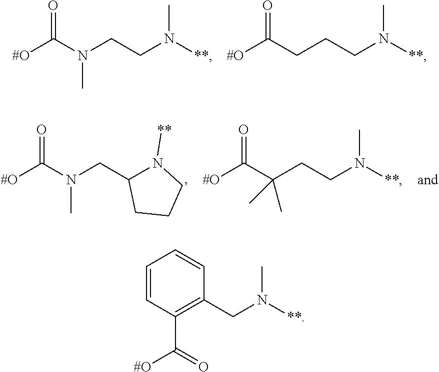

In some embodiments, L² is

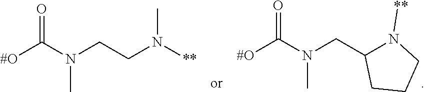

In some embodiments, p is 1 and T is a moiety represented by formula (IIc5):

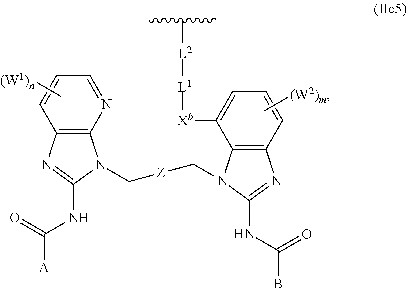

wherein:
T is coupled to the —C(O)OCR²R³— fragment of the compound represented by structural formula (I) via L²,
each instance of Wⁱ and W² is independently selected from alkyl, amino, amido, carboxylic acid, ester, and hydrazido, n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino, and —O(alkylene)guanidino,
L¹ is selected from alkylene, heteroalkylene, alkenylene, alkynylene, cycloalkylene, heterocyclylene, arylene, and heteroarylene,
L² is a bond, a linker moiety coupled to L¹ and comprising a nitrogen atom coupled to the —C(O)O(CR²R³)— fragment of the compound represented by structural formula (I), or a second linker, and

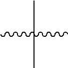

is the point of connection to —C(O)OCR²R³—.

In some embodiments, L² is a second linker comprising ^{#}OC(O)NR⁵-L⁴-NR⁶, ^{#}OC(O)-L⁴-NR⁶, or ^{#}OC(O)NR⁵-L⁴-(heterocyclylene), wherein:
the heterocyclylene comprises a nitrogen atom connected to the —C(O)O(CR²R³)— fragment of the compound represented by structural formula (I), and wherein # is the point of connection to L¹,
each instance of L⁴ is independently alkylene or arylalkylene,
each instance of R⁵ is independently selected from H, alkyl, and dialkylaminoalkyl, and
each instance of R⁶ is independently selected from H, alkyl, and dialkylaminoalkyl.

In some embodiments, p is 2 and T is a moiety represented by one of the following structural formulas:

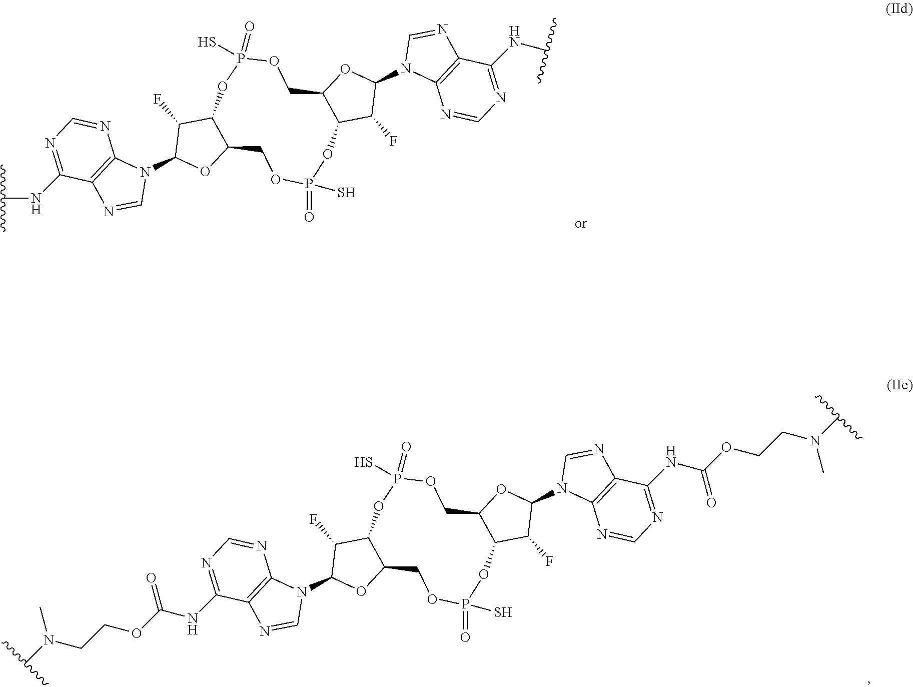 [SMILES: *Nc1ncnc2c1ncn2[C@@H]1O[C@@H]2COP(=O)(S)O[C@H]3[C@@H](F)[C@H](n4cnc5c(N*)ncnc54)O[C@@H]3COP(=O)(S)O[C@H]2[C@H]1F ; *N(C)CCOC(=O)Nc1ncnc2c1ncn2[C@@H]1O[C@@H]2COP(=O)(S)O[C@H]3[C@@H](F)[C@H](n4cnc5c(NC(=O)OCCN(*)C)ncnc54)O[C@@H]3COP(=O)(S)O[C@H]2[C@H]1F]

wherein

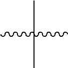

is the point of connection to —C(O)OCR²R³—.

In some embodiments, T is a moiety represented by structural formula (IIf), wherein T is coupled to each —C(O)OCR²R³— fragment of formula (I) independently via two of W¹, W², A, B, or L²:

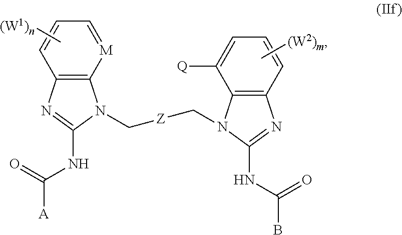

wherein:
M is N, C(X^{a}R^{a}), or C(X^{b}L¹L²-),
Q is —X^{a}R^{a} or -X^{b}L¹L²-,
each instance of W¹ and W² is independently selected from alkyl, amino, amido, carboxylic acid, ester, and hydrazido,
n and m are each independently 0, 1, 2, or 3,
Z is selected from alkylene, alkenylene, and alkynylene,
A and B are each independently 5-membered heteroaryl,
X^{a} and X^{b} are each independently selected from CH₂, NH, O, and S,
each instance of R^{a} is independently selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino, and —O(alkylene)guanidino,
each instance of L¹ is independently selected from alkylene, heteroalkylene, alkenylene, alkynylene, cycloalkylene, heterocyclylene, arylene, and heteroarylene,
each instance of L² is independently selected from a bond, a linker moiety coupled to the respective L¹ and comprising a nitrogen atom coupled to the respective —C(O)O(CR²R³)— fragment of the compound represented by structural formula (I), or a second linker.

In some embodiments, T is a moiety of formula (IId).

In some embodiments, T is a moiety of formula (IIe).

In some embodiments, the compound is

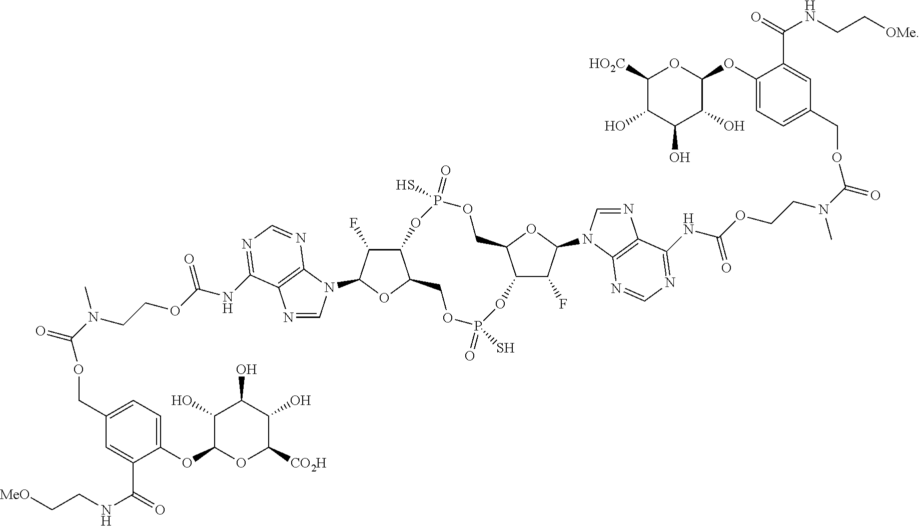 [SMILES: COCCNC(=O)c1cc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(NC(=O)OCCN(C)C(=O)OCc7ccc(O[C@@H]8O[C@H](C(=O)O)[C@@H](O)[C@H](O)[C@H]8O)c(C(=O)NCCOC)c7)ncnc65)O[C@@H]4CO[P@@](=O)(S)O[C@H]3[C@H]2F)ccc1O[C@@H]1O[C@H](C(=O)O)[C@@H](O)[C@H](O)[C@H]1O]

In some embodiments, T is a moiety of formula (IIf).

In some embodiments, T is coupled to each —C(O)OCR²R³— fragment via W¹ and W², and T is a moiety represented by formula (IIf1):

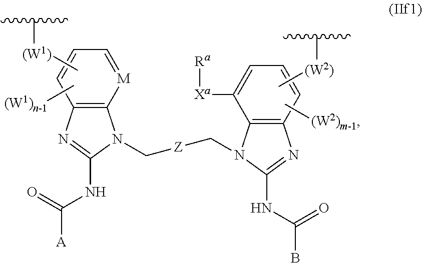

wherein each

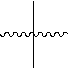

is a point of connection to the —C(O)O(CR²R³)— fragments of the compound represented by structural formula (I).

In some embodiments, T is coupled to each —C(O)OCR²R³— fragment via A and B, and T is a moiety represented by formula (IIf2):

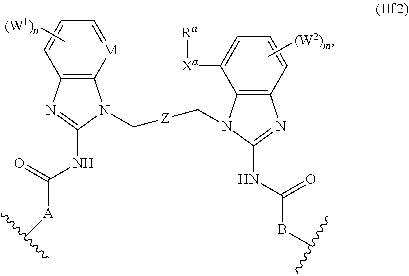

wherein each

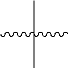

is a point of connection to the —C(O)O(CR²R³)— fragments of the compound represented by structural formula (I).

In some embodiments, T is coupled to each —C(O)OCR²R³— fragment via two L², and T is a moiety represented by formula (IIf3):

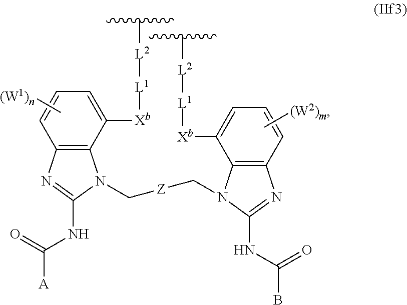

wherein each

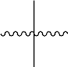

is a point of connection to the —C(O)O(CR²R³)— fragments of the compound represented by structural formula (I).

In some embodiments, L² is selected from a bond,

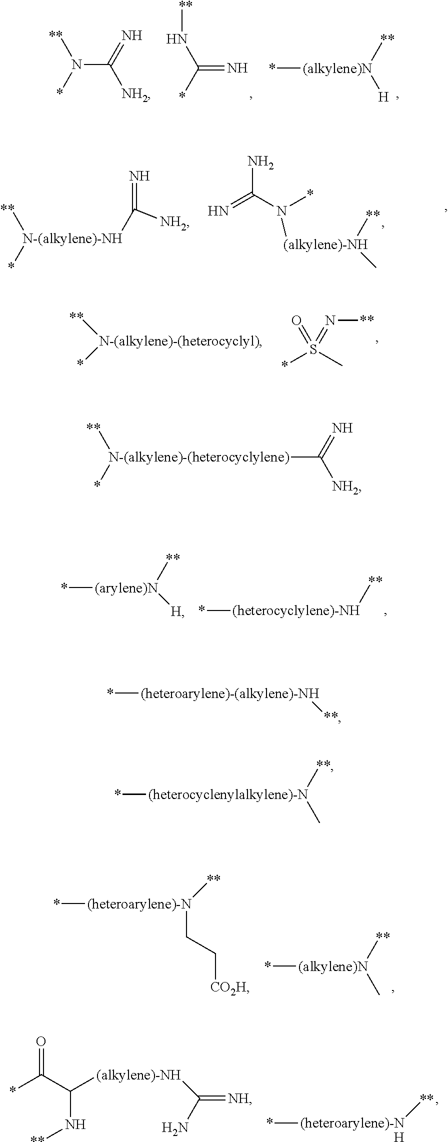

and *heterocyclylene** and wherein * is the point of connection to L¹ and ** is the point of connection to the —C(O)O(CR²R³)— fragment of the compound represented by structural formula (I).

In some embodiments, each L² is independently a second linker comprising ^{#}OC(O)NR⁵-L⁵-NR⁶, ^{#}OC(O)-L⁴-NR⁶, or ^{#}OC(O)NR⁵-L⁵-(heterocyclylene), wherein the heterocyclylene comprises a nitrogen atom connected to the respective —C(O)O(CR²R³)— fragment of the compound represented by structural formula (I), wherein # is the point of connection to the respective L¹,
each L⁵ is independently alkylene or aralkylene, and
each R⁵ and each R⁶ are each independently selected from H, alkyl, and dialkylaminoalkyl.

In some embodiments, p is 1 and T is a moiety represented by formula (III):

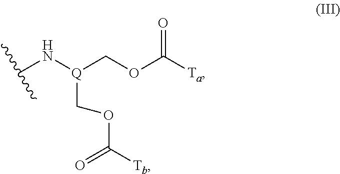

wherein Q is a branched linker moiety,
Tₐ and T_{b} are each independently a moiety comprising a Stimulator of Interferon Genes (STING) agonist, and

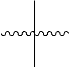

is a point of connection to the —C(O)O(CR²R³)— fragment of the compound represented by structural formula (III).

In some embodiments, T is a moiety represented by formula (IIIa):

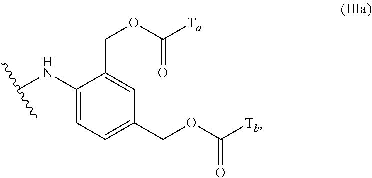

wherein:

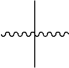

is the point of connection to the —C(O)O(CR²R³)— fragment of the compound represented by structural formula (I),
Ta and Tb are each independently a moiety represented by formula (IV):

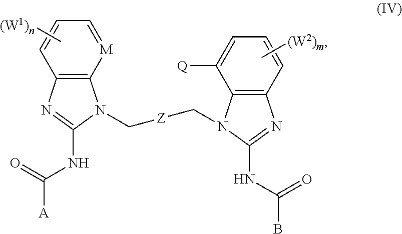

wherein:
Ta and Tb are each independently coupled to the —C(O)OCR²R³— fragment of formula (I) via W¹, W², A, B, or L²
M is N, C(X^{a}R^{a}) or C(X^{b}L¹L²-),
Q is —X^{a}R^{a} or -X^{b}L¹L²-,
each instance of W¹ and W² is independently selected from alkyl, amino, amido, carboxylic acid, ester, and hydrazido, e.g. —C(O)NH-(alkyl)-N(alkyl)-;
n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino, and —O(alkylene)guanidino,
L¹ is selected from alkylene, heteroalkylene, alkenylene, alkynylene, cycloalkylene, heterocyclylene, arylene, and heteroarylene,
L² is a bond, a linker moiety coupled to L¹ and comprising a nitrogen atom coupled to the —C(O)O(CR²R³)— fragment of the compound represented by structural formula (IIIa) or a second linker.

In some embodiments, A and B are each independently substituted or unsubstituted pyrazole or substituted or unsubstituted oxazole.

In some embodiments, A and B are each independently substituted pyrazole or substituted oxazole, wherein the pyrazole and oxazole are each substituted with two C₁₋₃ alkyls.

In some embodiments, p is 1 and T is a moiety represented by formula (IIIb):

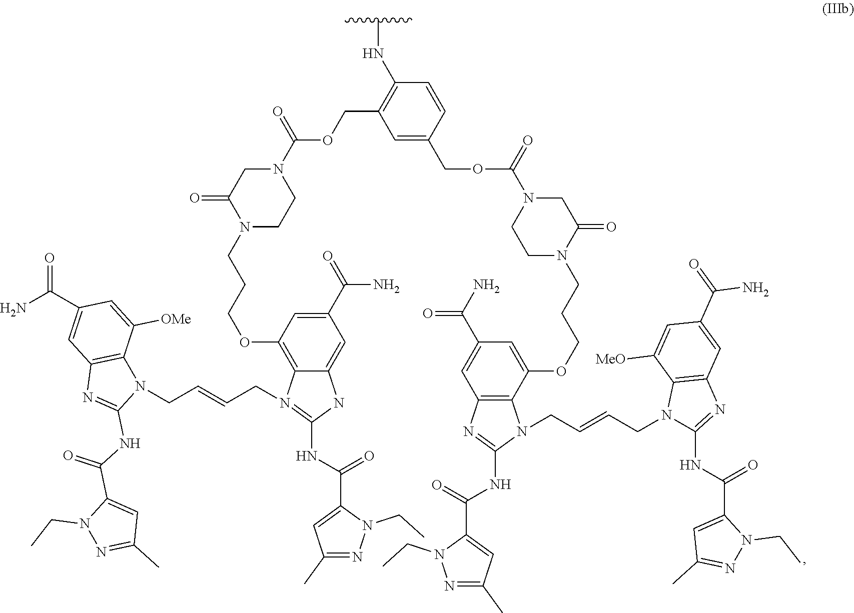

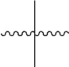

is the point of connection to the —C(O)O(CR²R³)— fragment of the compound represented by structural formula (I).

In some embodiments, A and B are each independently a 5-membered heteroaryl optionally substituted by 1 to 4 groups independently selected from halogen, OH, CN, NO₂, amine, amide, amidine; —(CH₂)_{q}NR^{w}R^{w1}; C₁₋₁₀ alkyl, C₂₋₁₀ alkenyl, and C₂₋₁₀ alkynyl, wherein: each q is independently selected from 0, 1, 2 or 3, and R^{w} and R^{w1} are each independently selected from hydrogen, C₁₋₁₀ alkyl, C₂₋₁₀ alkenyl, and C₂₋₁₀ alkynyl.

For example, A and B may be each independently selected from pyrazole, imidazole, oxazole, isoxazole, thiazole, and isothiazole. In certain preferred embodiments, A and B are each pyrazole.

In some embodiments, A and B are each independently substituted or unsubstituted pyrazole. For example, in certain preferred embodiments, A and B are each pyrazole substituted with two C₁₋₃ alkyls.

In some embodiments, A and B are each independently substituted or unsubstituted oxazole. For example, in certain preferred embodiments, A and B are each oxazole substituted with two C₁₋₃ alkyls.

In some embodiments, A and B are each independently substituted or unsubstituted pyrazole or substituted or unsubstituted oxazole.

In some embodiments, A and B are each independently substituted pyrazole or substituted oxazole, wherein the pyrazole and oxazole are each substituted with two C₁₋₃ alkyls.

In some embodiments, A and B are each independently represented by one of the following structural formulas:

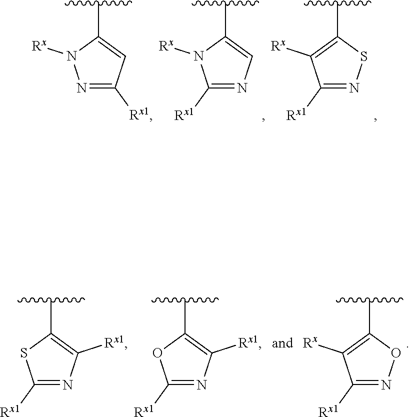

wherein R^{x} and R^{x1} are each independently selected from hydrogen, C₁₋₅ alkyl, C₁₋₅ haloalkyl, halogen, OH, —OP(O)(R^{y}R^{y1})₂, —OR, —NR^{y}R^{y1}, —OCOR^{y}, —CO₂R^{y}, —SOR^{y}, —SO₂R^{y}, —CONR^{y}R^{y1}, —SO₂NR^{y}R^{y1}, —OCONR^{y}R^{y1}, —NR^{y}COR^{y1}, —NR^{y}SOR^{y1}, —NR^{y}CO₂R^{y1}, and —NR^{y}SO₂R^{y1}, and wherein R^{y} and R^{y1} are each independently selected from hydrogen, C₁₋₁₀ alkyl, C₂₋₁₀ alkenyl, and C₂₋₁₀ alkynyl.

For example, A and B may be each independently represented by one of the following structural formulas:

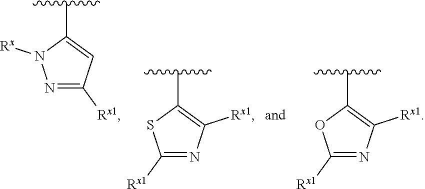

In some preferred embodiments, A and B are each represented by the following structural formula:

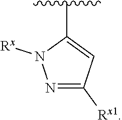

In some embodiments, A and B are represented by the following structural formula:

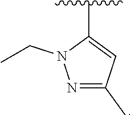 [SMILES: *c1cc(C)nn1CC]

In some embodiments, A and B are represented by the following structural formula:

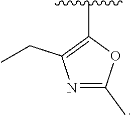 [SMILES: *c1oc(C)nc1CC]

In some embodiments, A and B are represented by the following structural formula:

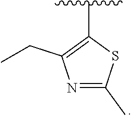 [SMILES: *c1sc(C)nc1CC]

In some embodiments, each instance of the first linker is independently selected in each instance from *(alkylene)O(alkylene)**, *(heteroalkylene)**, *(alkylene)**, *(heteroaralkylene)**, *(heteroalkylene)(heterocyclylene)**, *CH₂CH₂C(O)NHCH**, *(CH₂CH₂O)ₜ—**, and *(alkylene)(heteroarylene)(CH₂CH₂O)ₜ**,
wherein * indicates the point of connection to the —C(O)NH— fragment of the compound represented by structural formula (I), ** indicates the point of connection to U, and t represents an integer from 1-15.

In some embodiments, each instance of the first linker is independently selected from *(CH₂CH₂O)ₜCH₂**, *(CH₂CH₂O)₂CH₂CH₂N(CH₃)CH₂**, *(CH₂CH₂O)₂CH₂CH₂N(CH₃)**, *(CH₂CH₂O)ₜ**, *(CH₂CH₂O)ₜCH₂CH₂NH**, *CH₂CH₂**, *(CH₂CH₂O)ₜCH₂**, *(CH₂CH₂O)ₜCH₂CH₂ heterocyclylene**, *(CH₂CH₂O)ₜCH₂**, *(CH₂CH₂O)ₜCH₂CH₂**, and *(CH₂CH₂O)ₜNH**.

In some embodiments, t represents an integer from 1-6. In other embodiments, t represents an integer from 1-3.

In some embodiments, U is H. In other embodiments, U is a reactive group. Any suitable reactive group may be used, such that an additional moiety with a complementary reactive group may be coupled the compound of formula (I) through reaction with U. In some such embodiments, the reactive group is an amino, azido, acetylenyl, —COOH, —P(O)(OH)₂, or —OH. In some embodiments, U is alkylamino, heterocyclyl, or alkoxy. In some embodiments, U is a heterocyclyl comprising at least one nitrogen, which is the point of connection to L^{u}. In some embodiments, U is —N(alkyl)₂, for example —N(CH₃)₂. In some embodiments, U is —(O)alkyl, for example, —OCH₃.

In some embodiments, U is —NH(CH₃), —N(CH₃)₂, —OCH₃, or

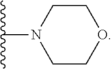 [SMILES: *N1CCOCC1]

In some embodiments, the compound is selected from

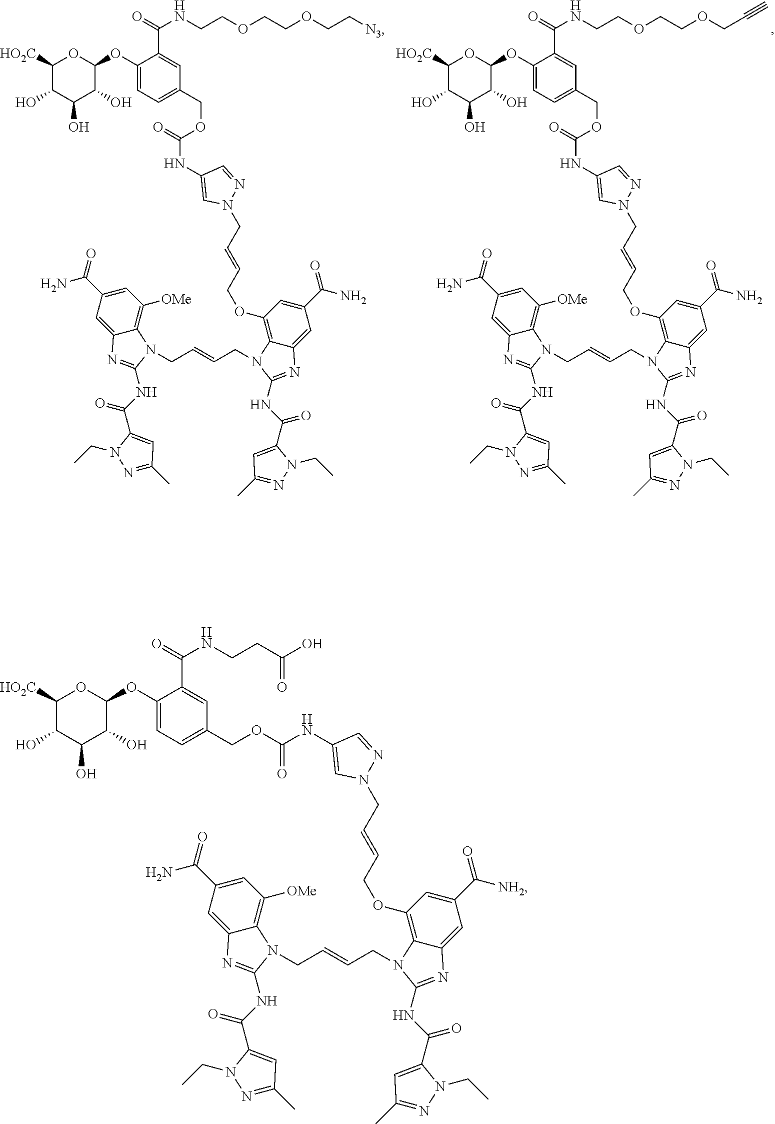

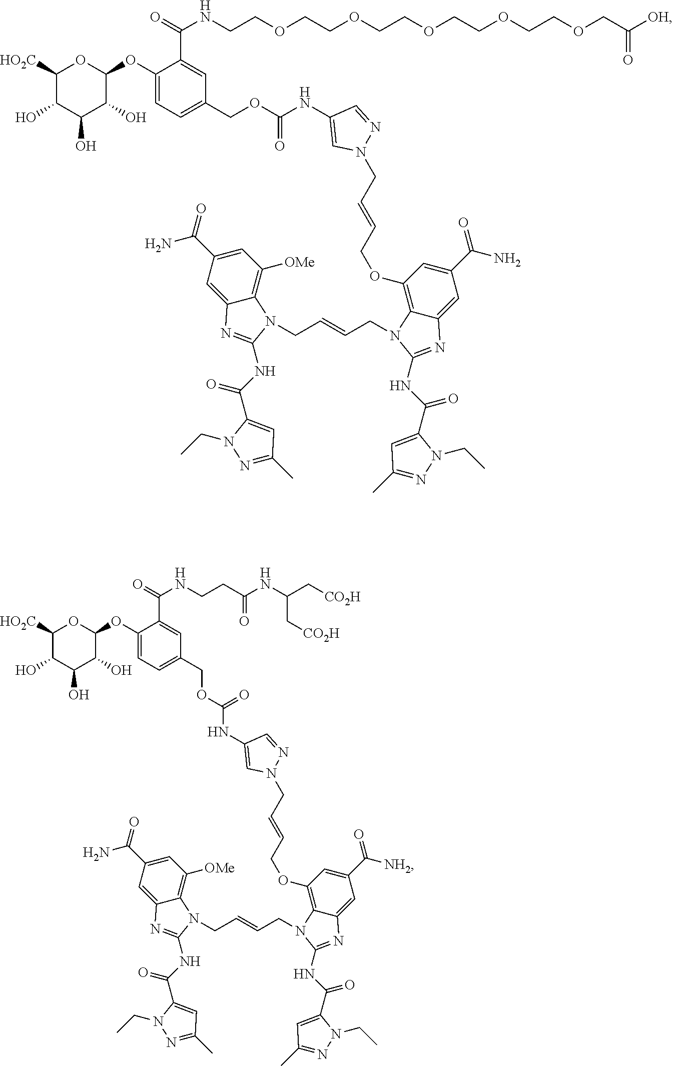 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCOCCOCCOCC(=O)O)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCC(=O)NC(CC(=O)O)CC(=O)O)c4)cn3)c21]

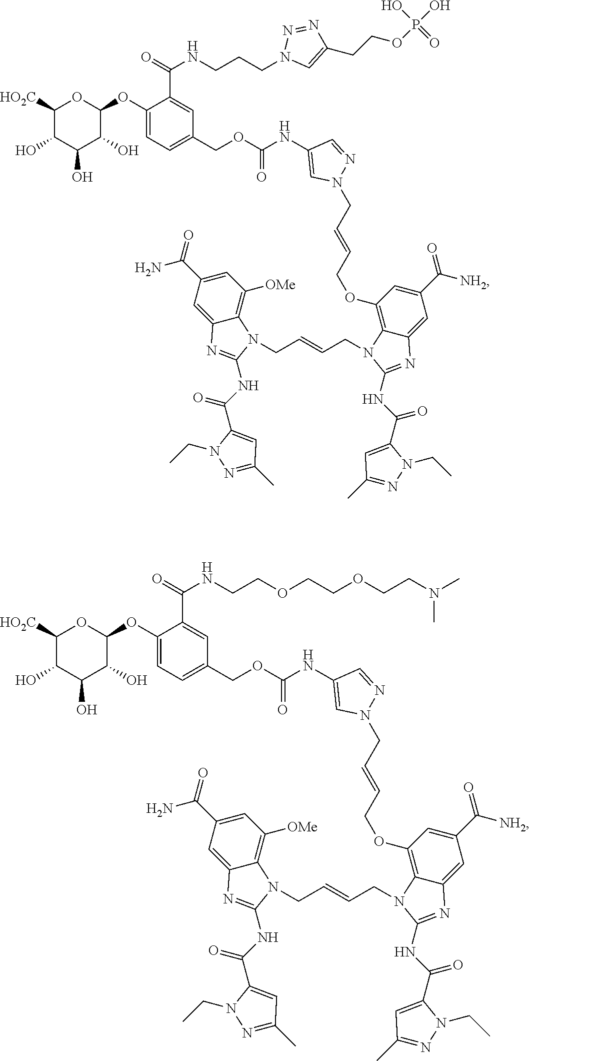 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCCn5cc(CCOP(=O)(O)O)nn5)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN(C)C)c4)cn3)c21]

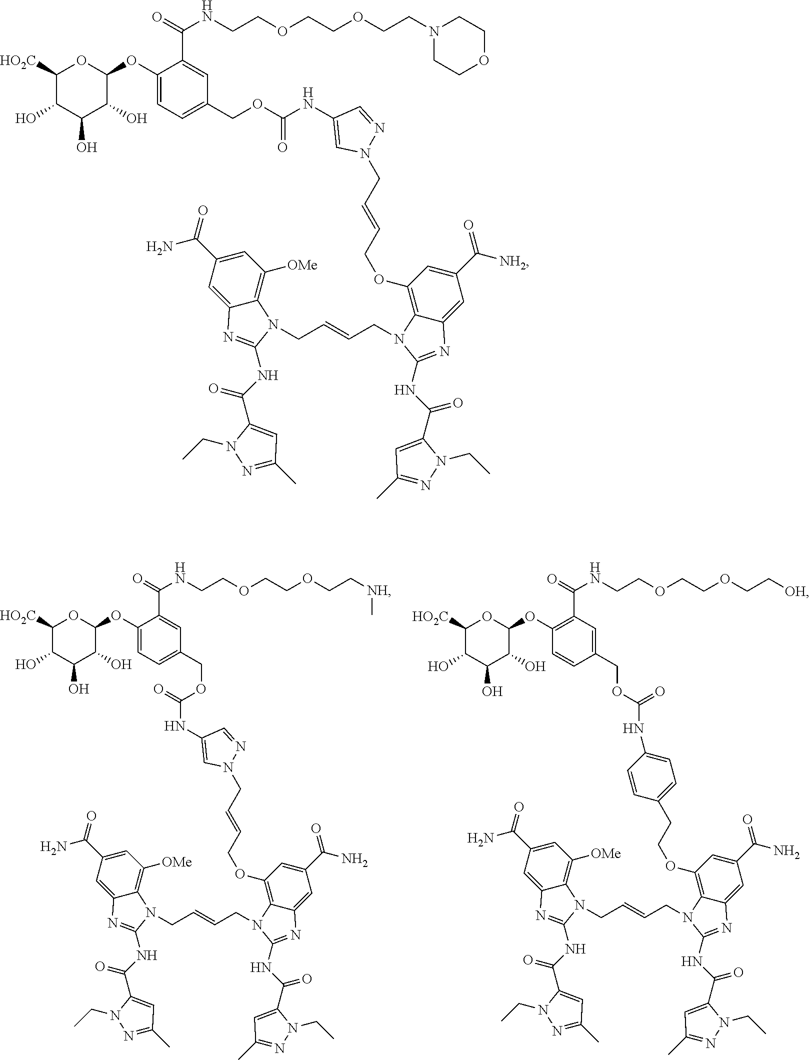

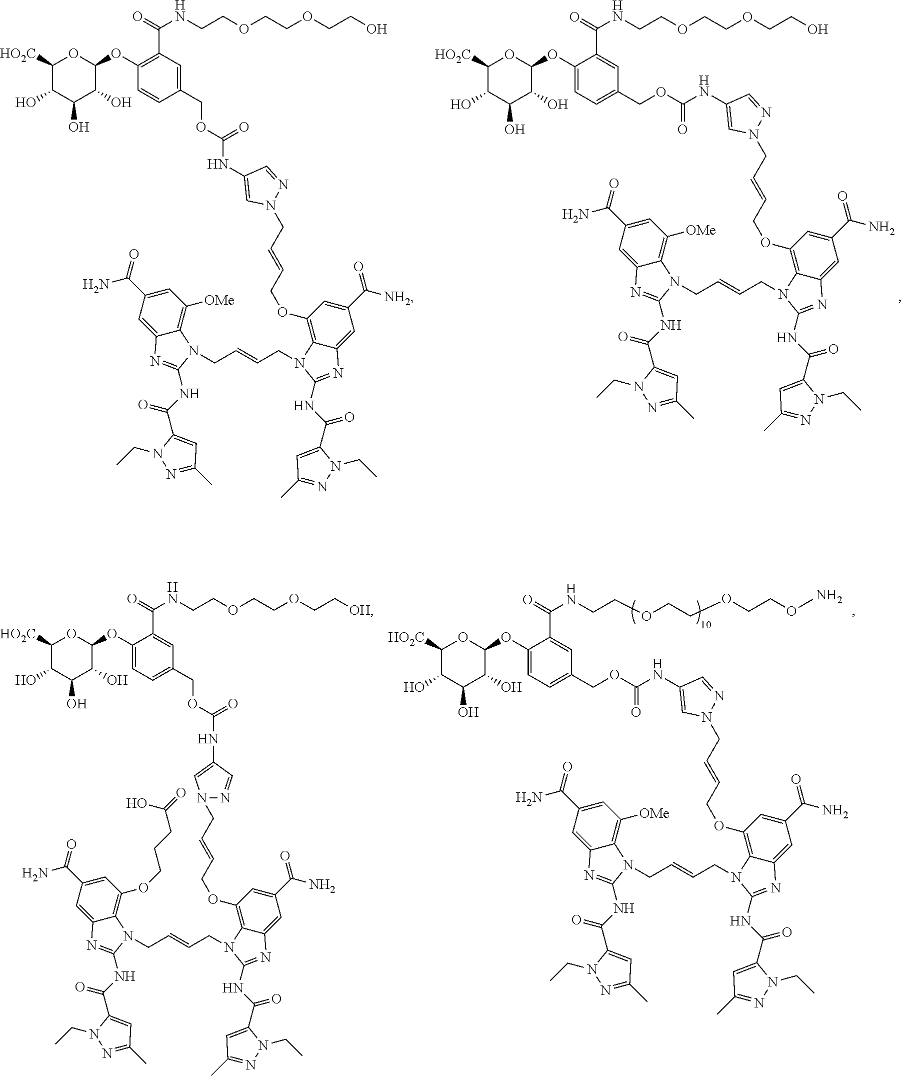

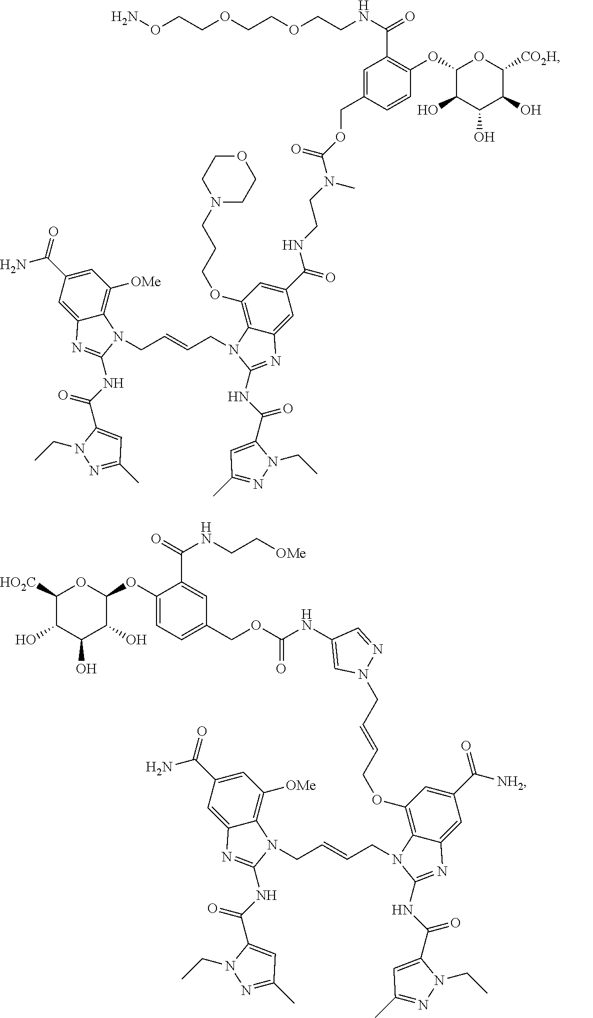 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NCCN(C)C(=O)OCc3ccc(O[C@@H]4O[C@H](C(=O)O)[C@@H](O)[C@H](O)[C@H]4O)c(C(=O)NCCOCCOCCON)c3)cc(OCCCN3CCOCC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOC)c4)cn3)c21]

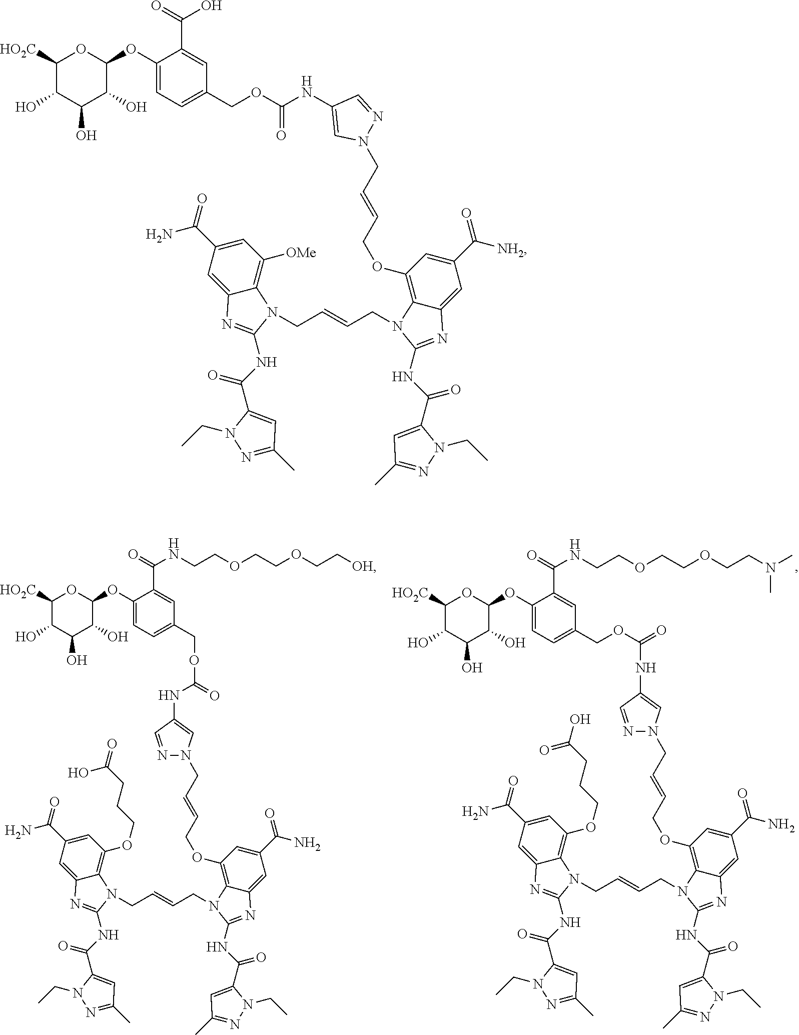

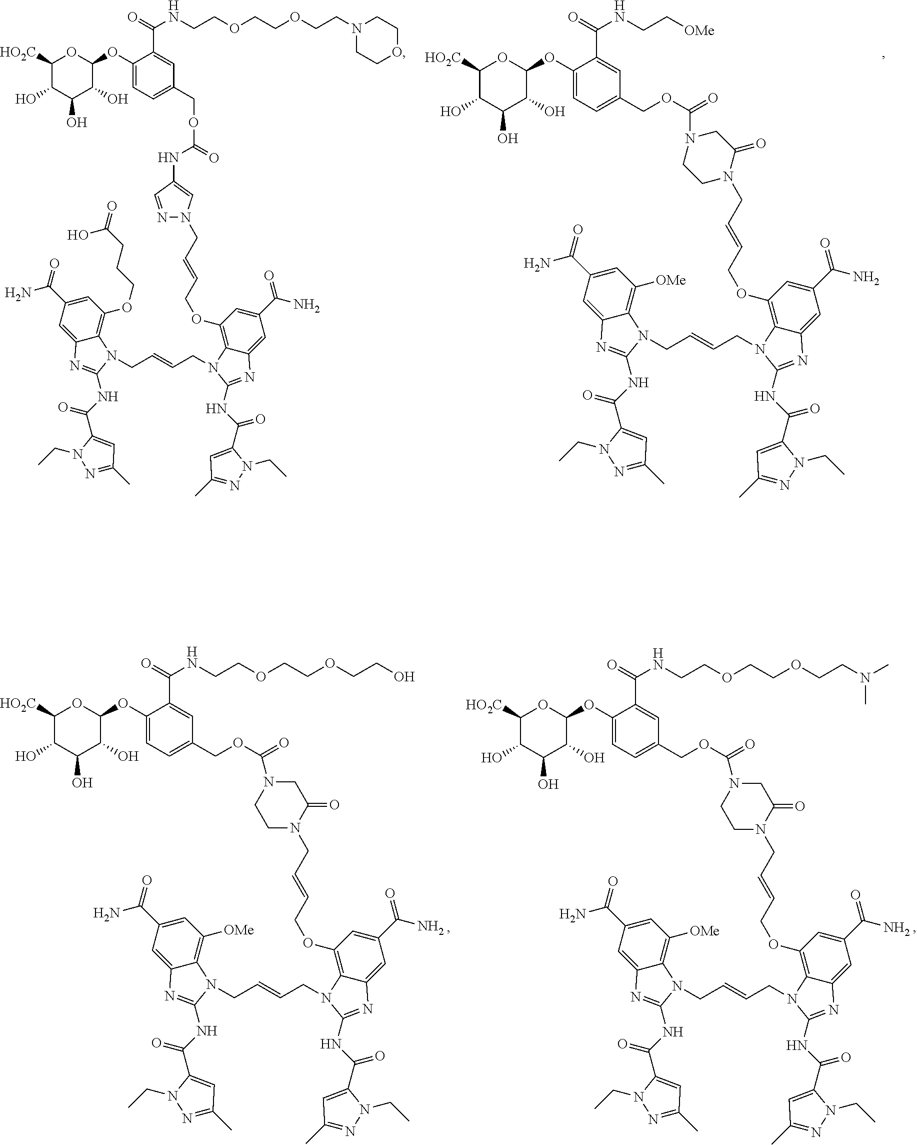

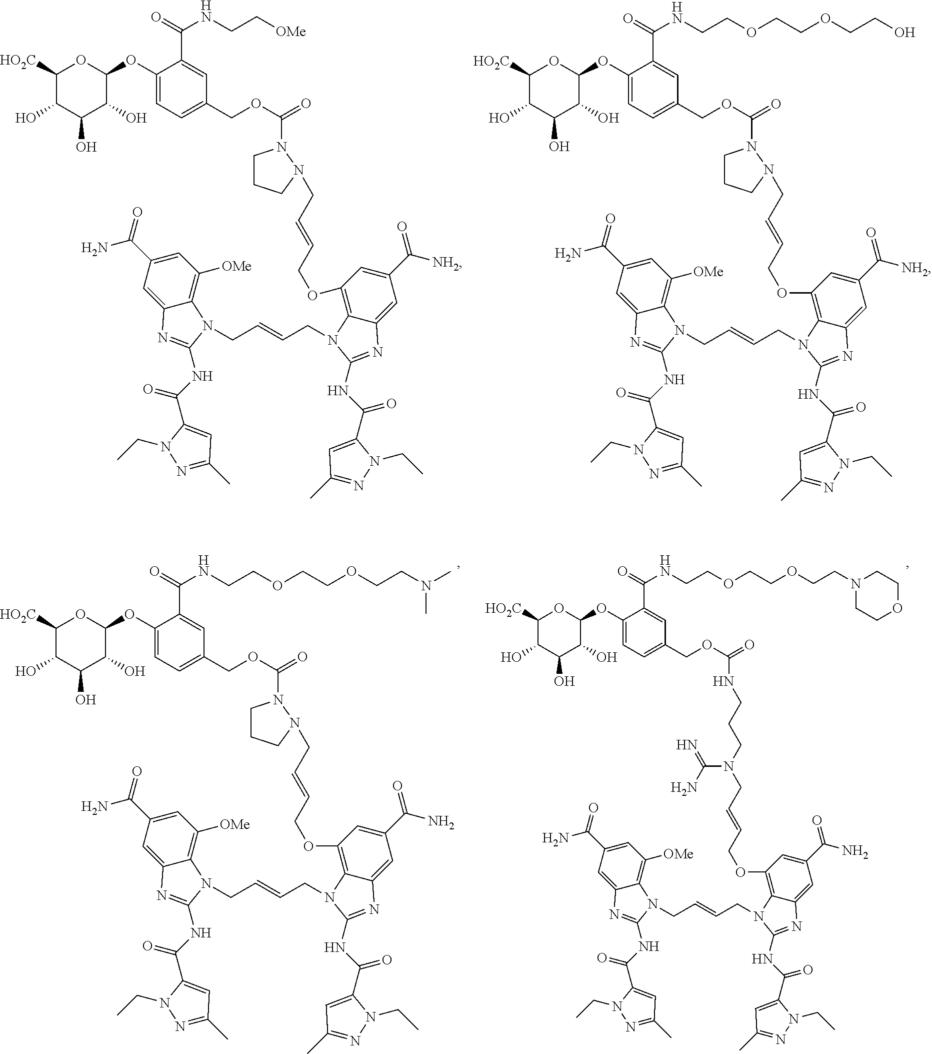

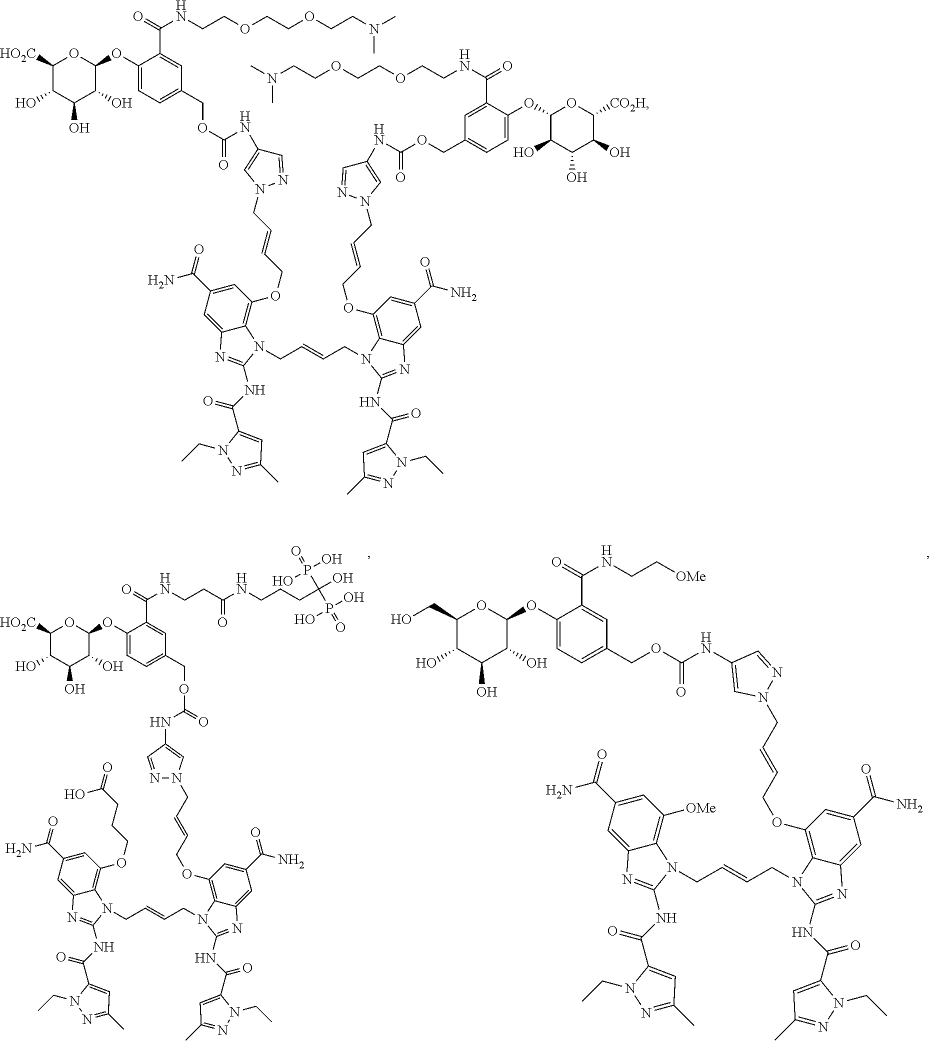

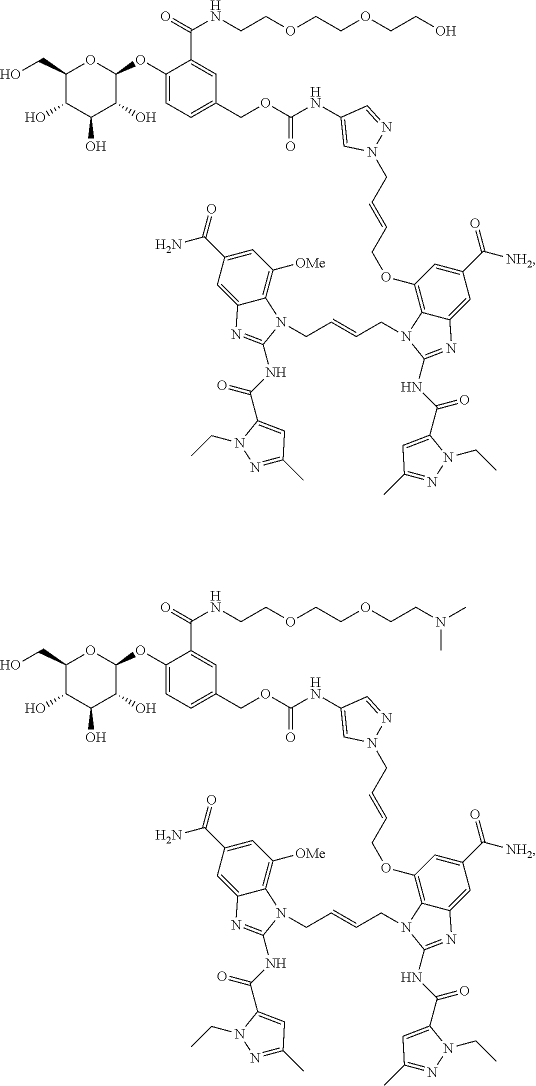 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](CO)[C@@H](O)[C@H](O)[C@H]5O)c(C(=O)NCCOCCOCCO)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](CO)[C@@H](O)[C@H](O)[C@H]5O)c(C(=O)NCCOCCOCCN(C)C)c4)cn3)c21]

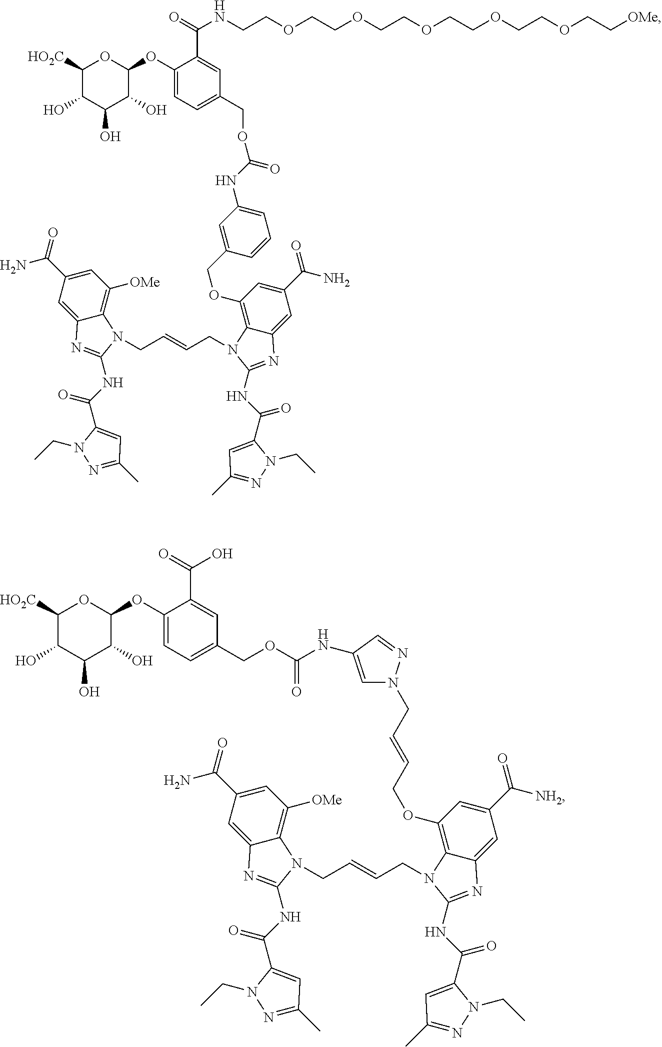 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCc3cccc(NC(=O)OCc4ccc(O[C@@H]5O[C@H](C(=O)O)[C@@H](O)[C@H](O)[C@H]5O)c(C(=O)NCCOCCOCCOCCOCCOCCOC)c4)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)O)c4)cn3)c21]

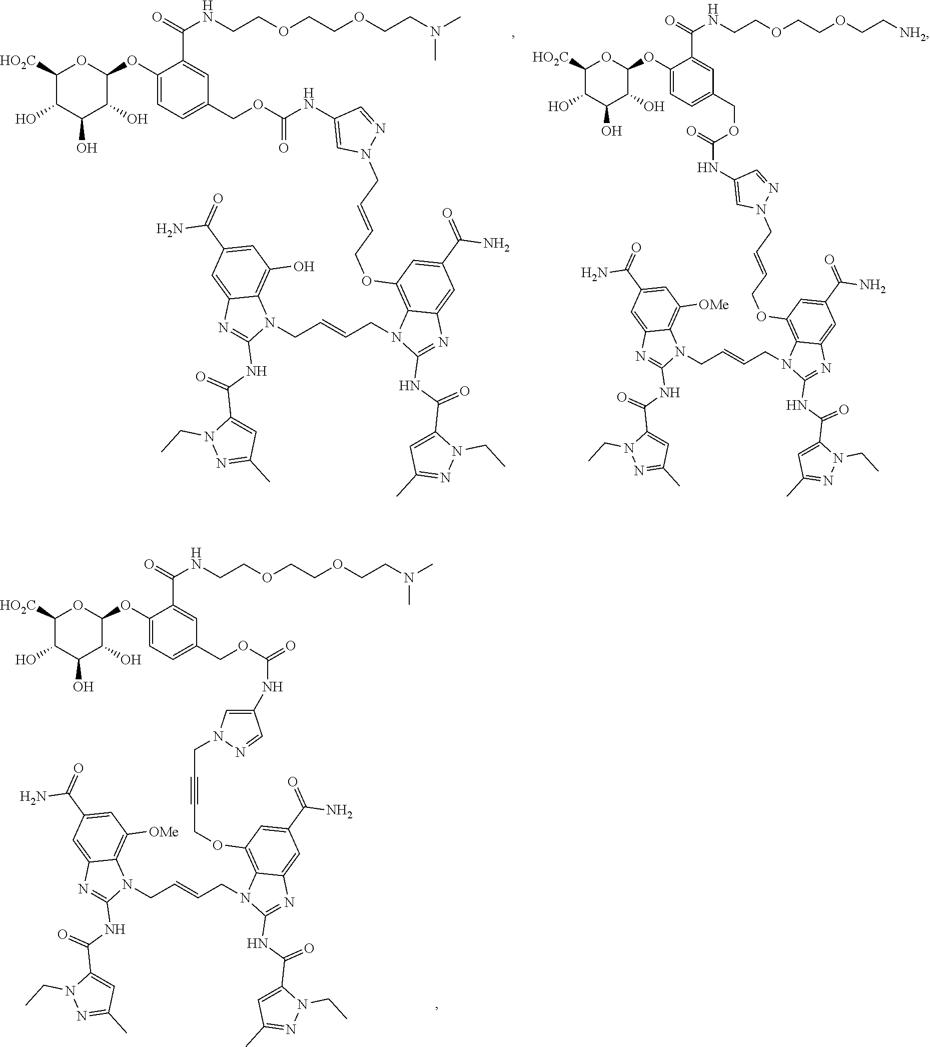

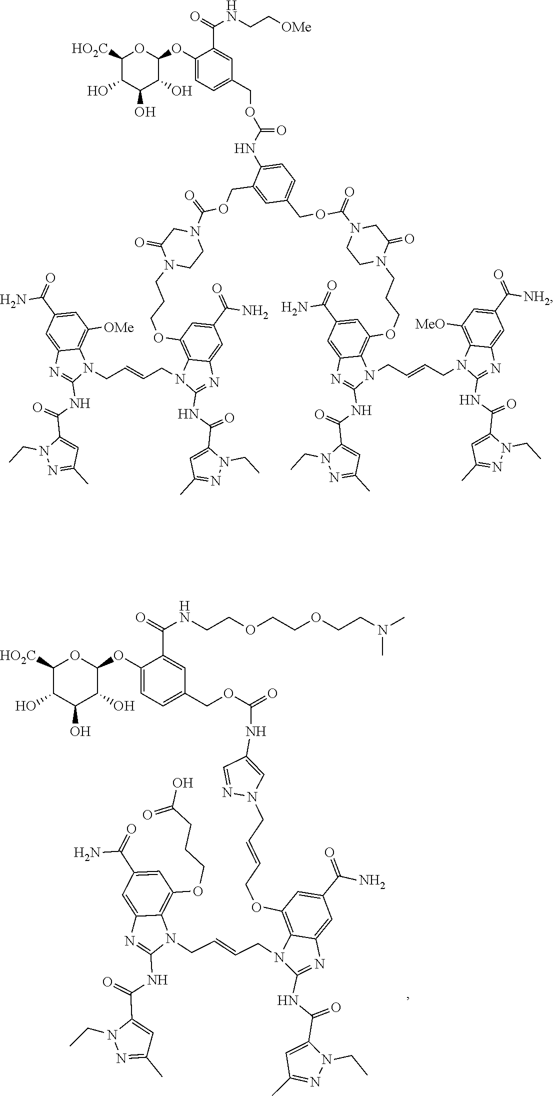

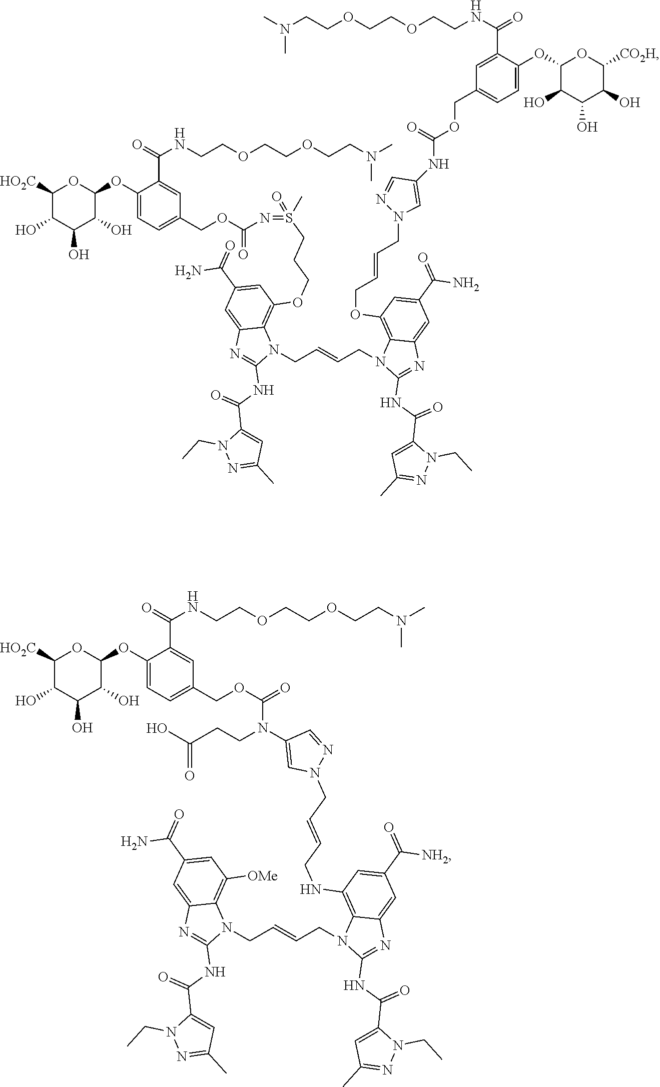 [SMILES: CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN(C)C)c4)cn3)c2n1C/C=C/Cn1c(NC(=O)c2cc(C)nn2CC)nc2cc(C(N)=O)cc(OCCCS(C)(=O)=NC(=O)OCc3ccc(O[C@@H]4O[C@H](C(=O)O)[C@@H](O)[C@H](O)[C@H]4O)c(C(=O)NCCOCCOCCN(C)C)c3)c21 ; CCn1nc(C)cc1C(=O)Nc1nc2cc(C(N)=O)cc(NC/C=C/Cn3cc(N(CCC(=O)O)C(=O)OCc4ccc(O[C@@H]5O[C@H](C(=O)O)[C@@H](O)[C@H](O)[C@H]5O)c(C(=O)NCCOCCOCCN(C)C)c4)cn3)c2n1C/C=C/Cn1c(NC(=O)c2cc(C)nn2CC)nc2cc(C(N)=O)cc(OC)c21]

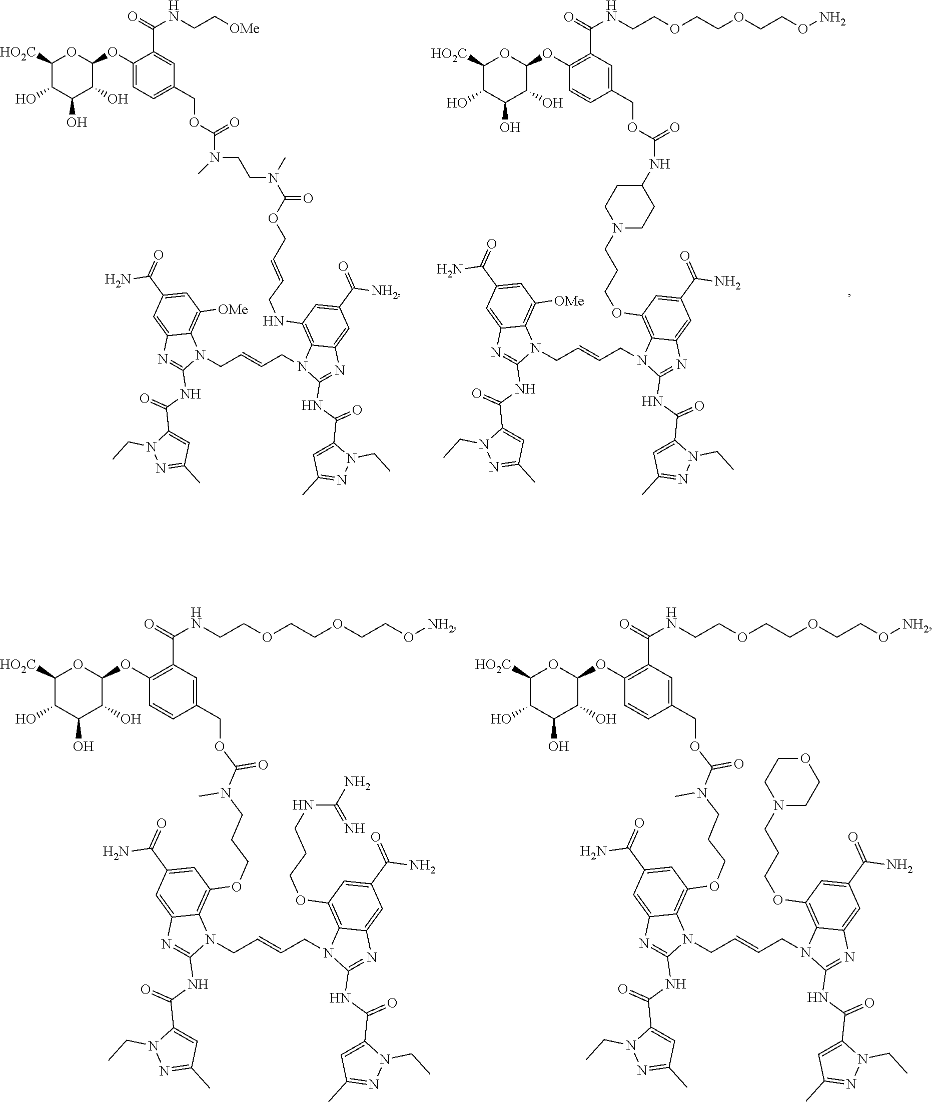

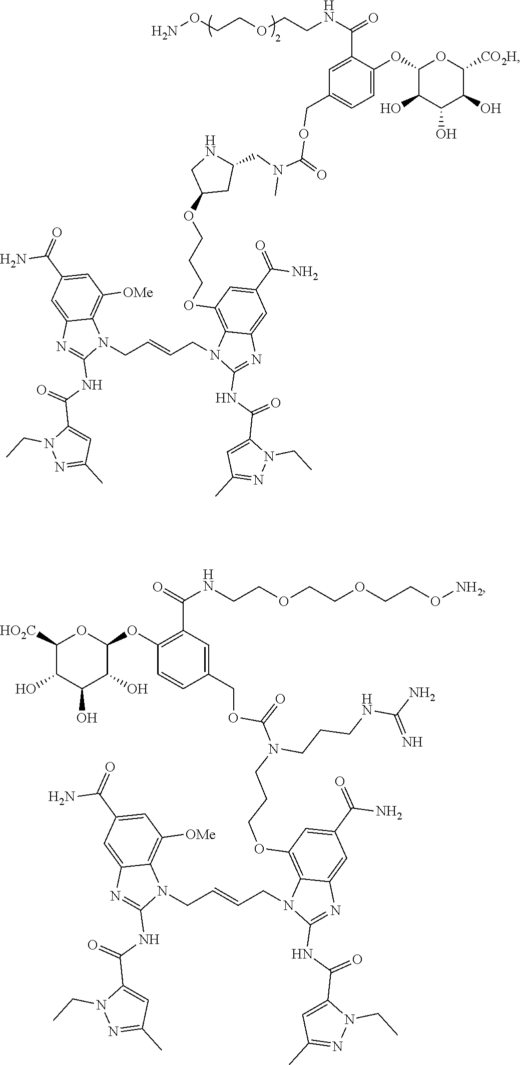

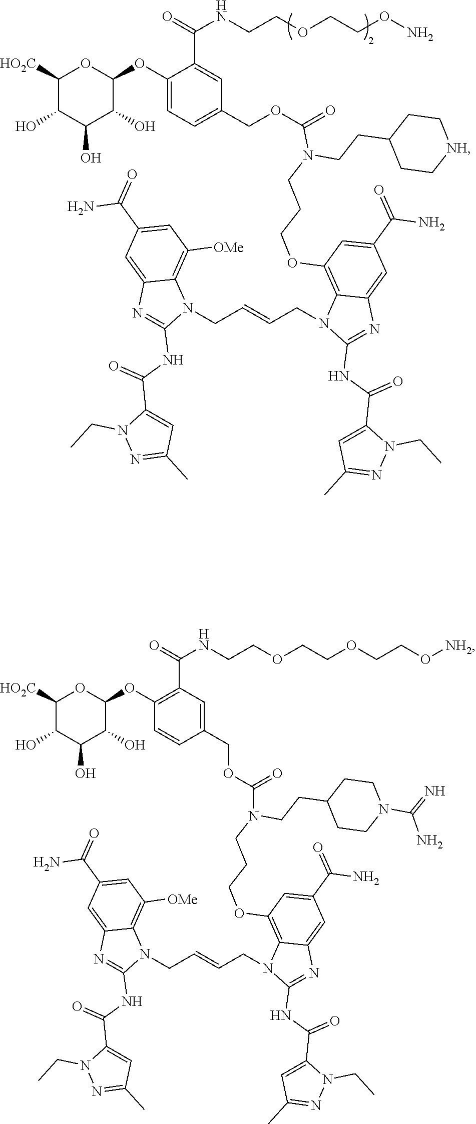

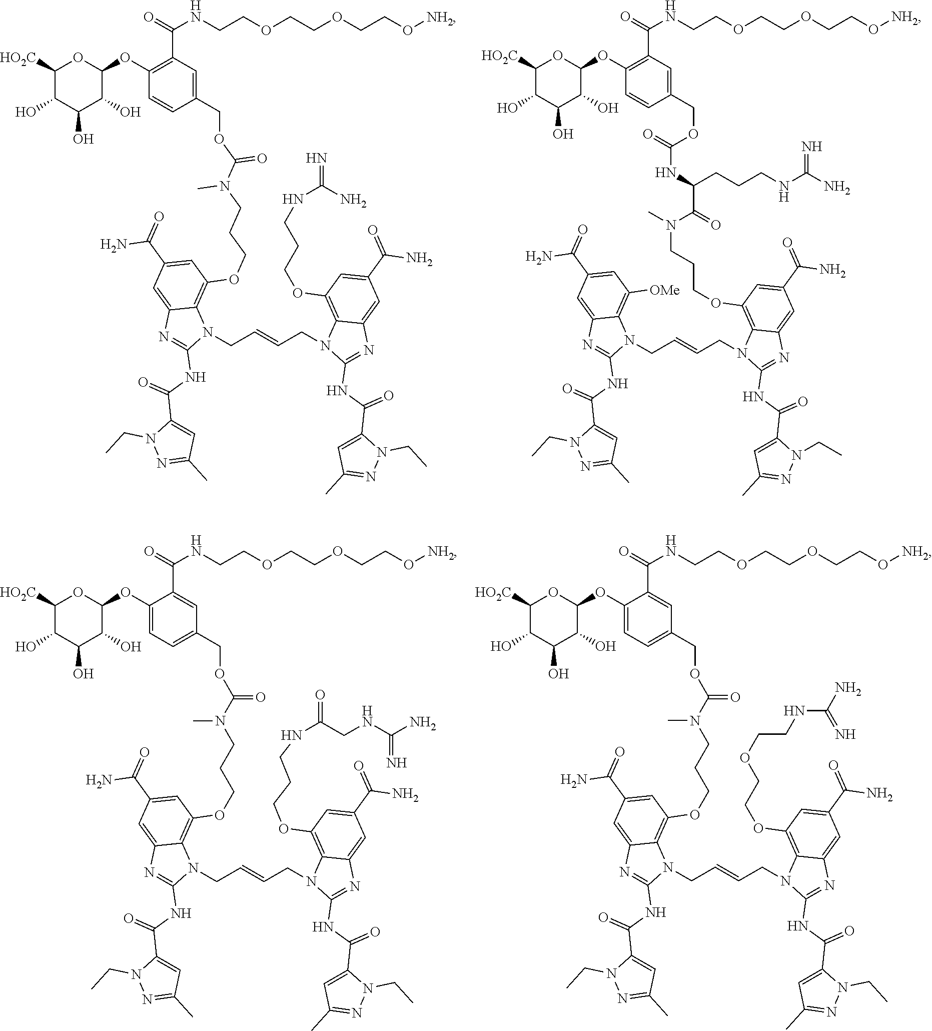

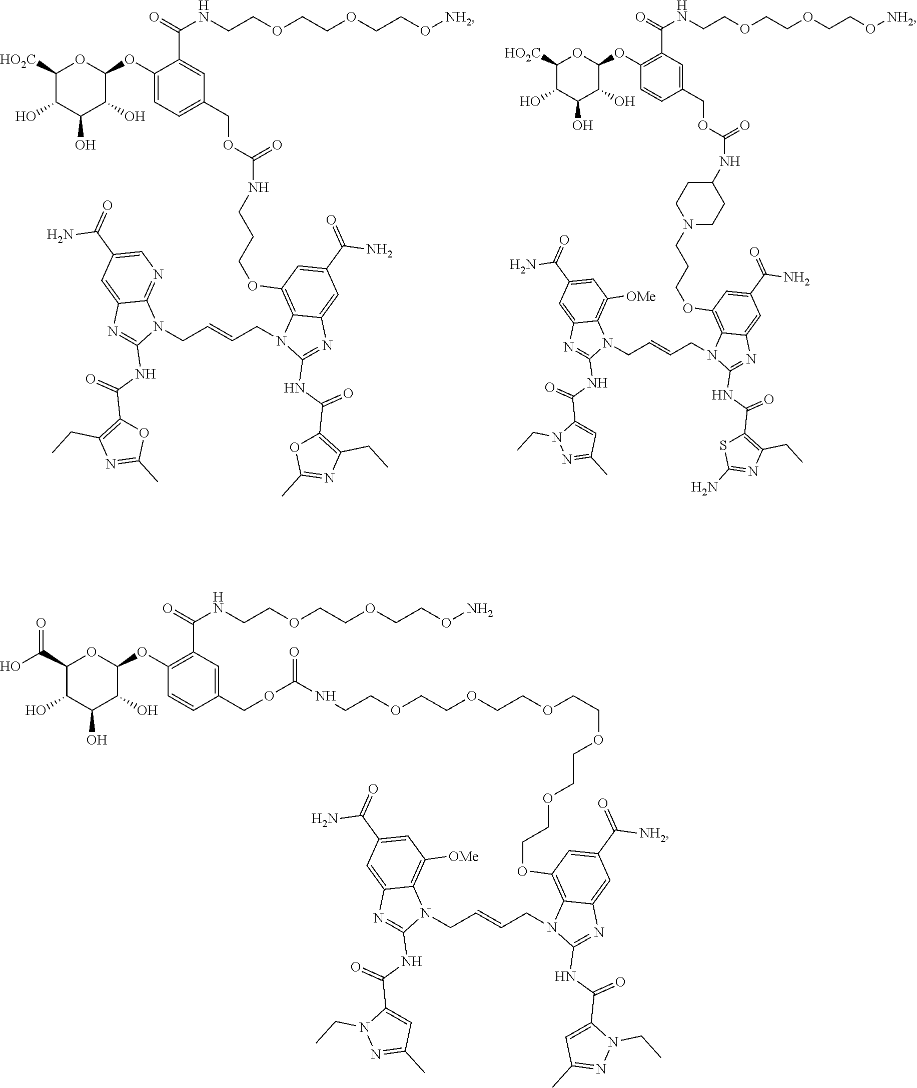

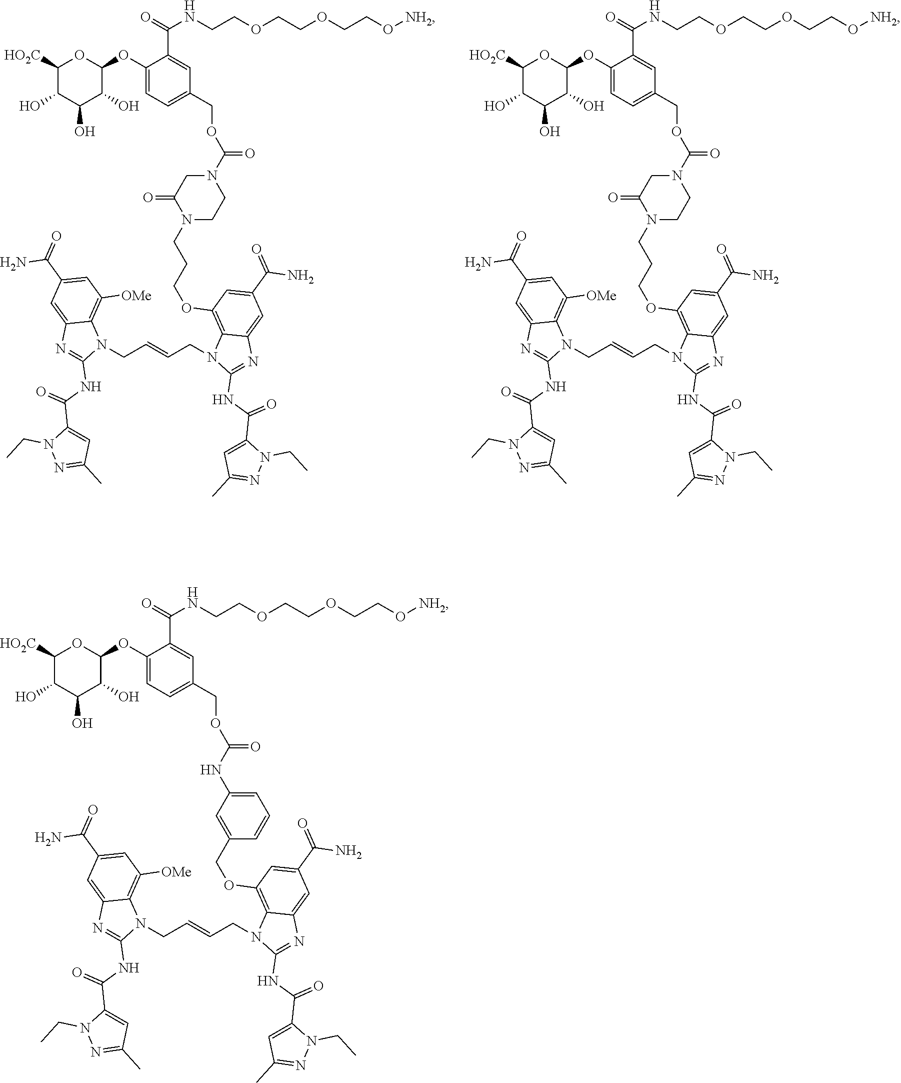

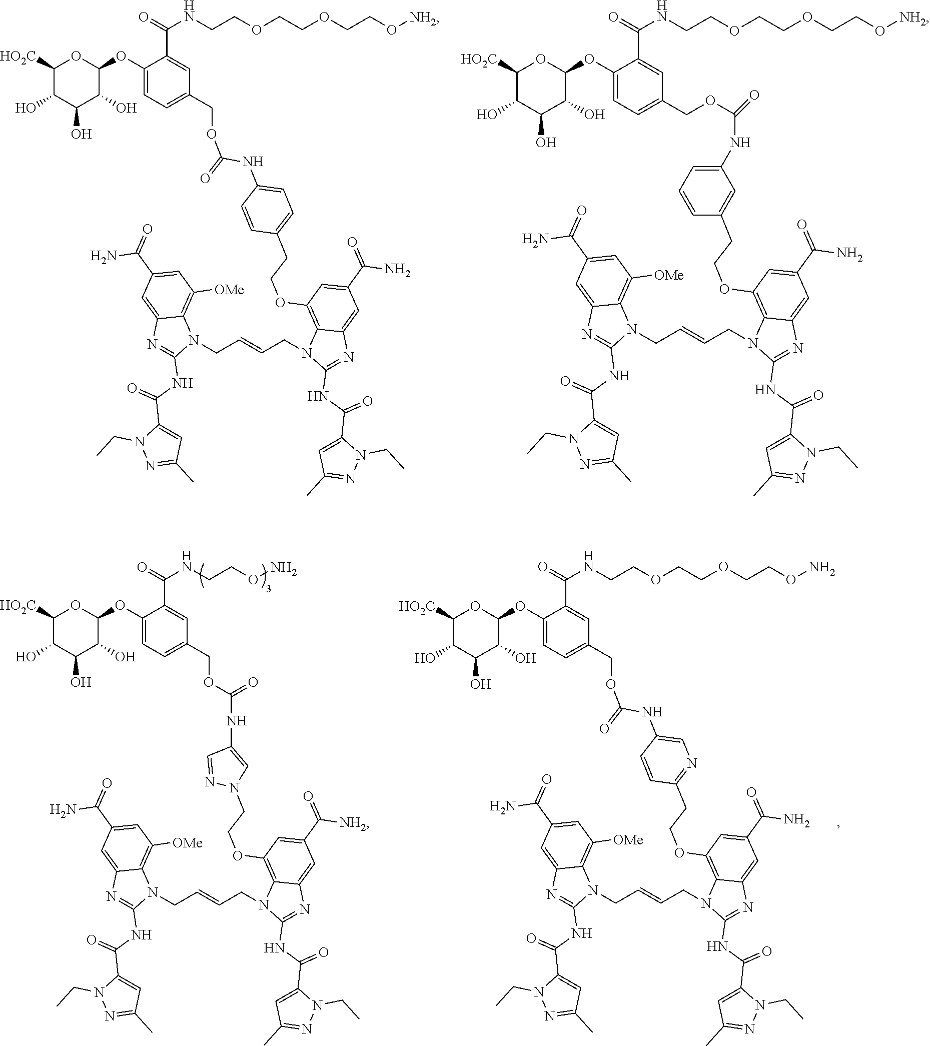

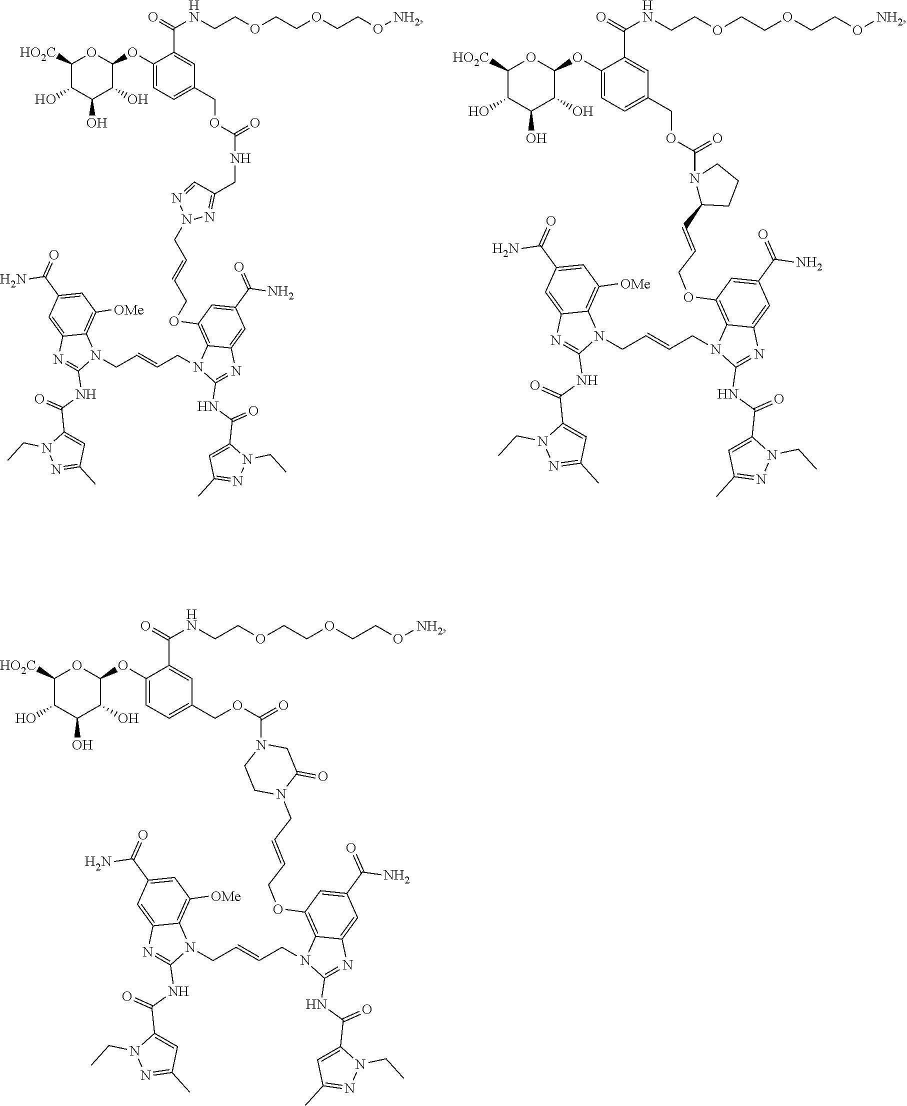

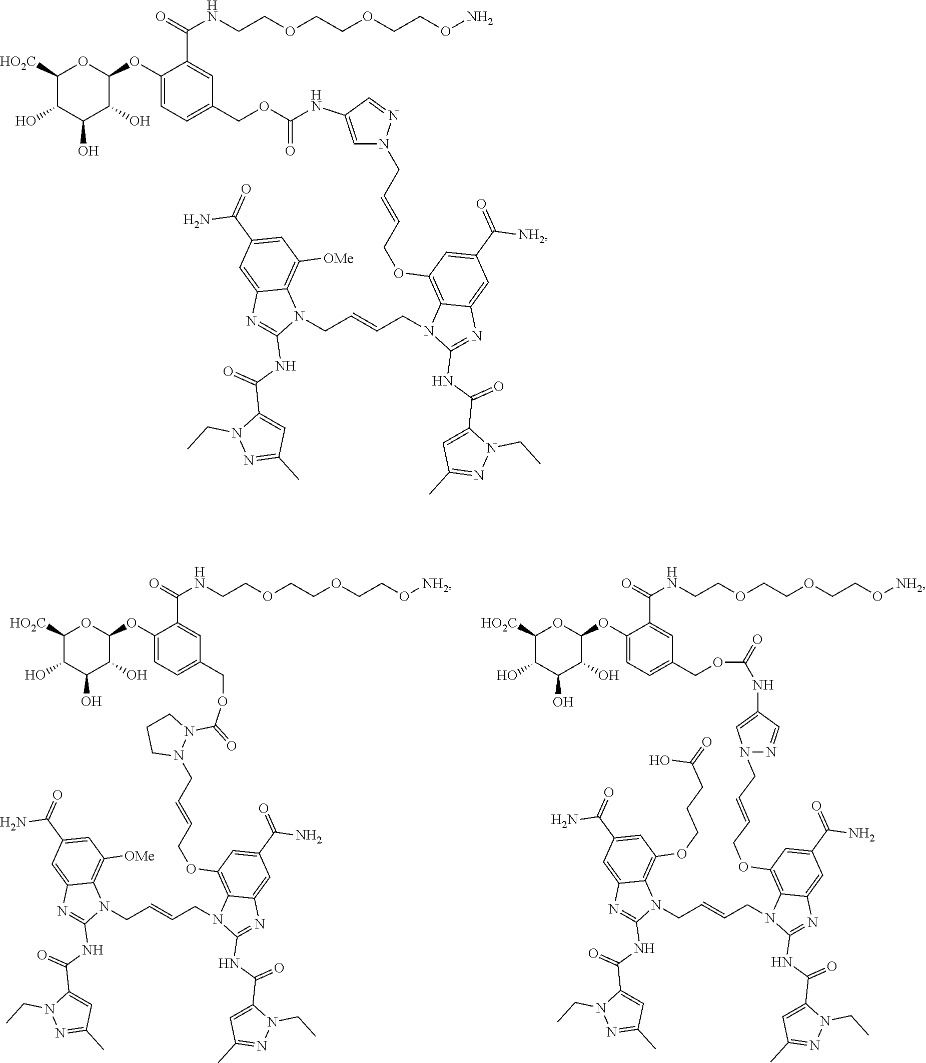

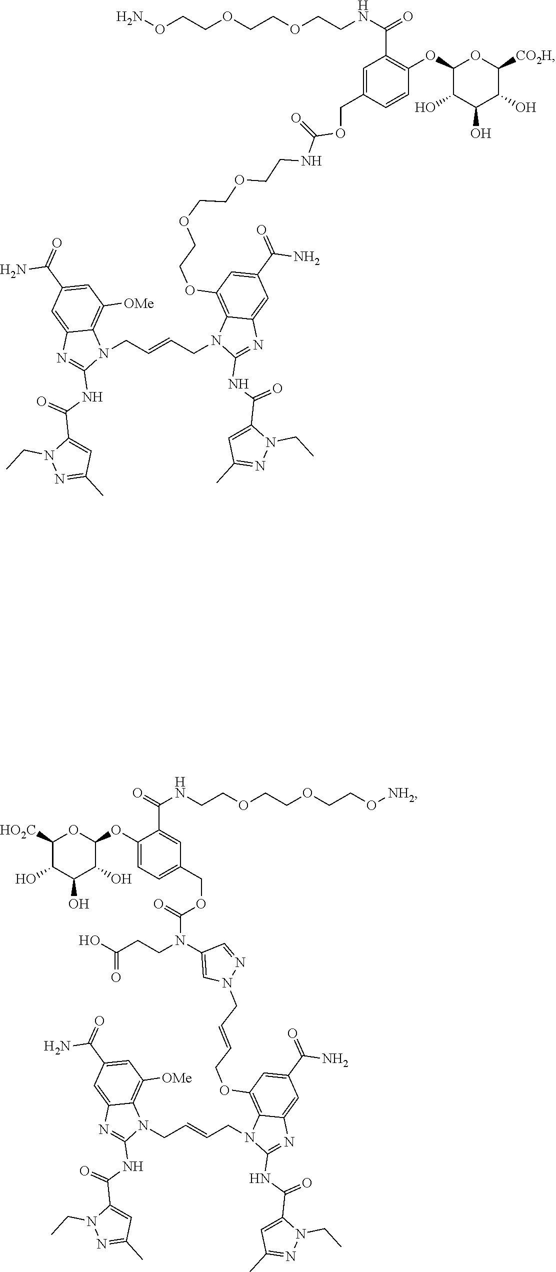 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCOCCOCCNC(=O)OCc3ccc(O[C@H]4O[C@@H](C(=O)O)[C@H](O)[C@@H](O)[C@@H]4O)c(C(=O)NCCOCCOCCON)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(N(CCC(=O)O)C(=O)OCc4ccc(O[C@@H]5O[C@H](C(=O)O)[C@@H](O)[C@H](O)[C@H]5O)c(C(=O)NCCOCCOCCON)c4)cn3)c21]

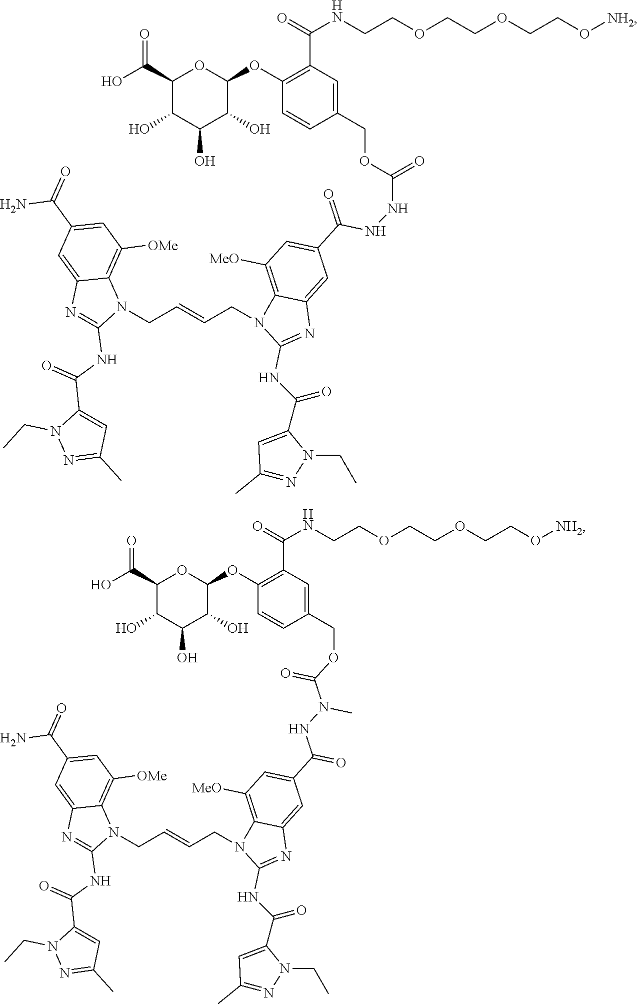

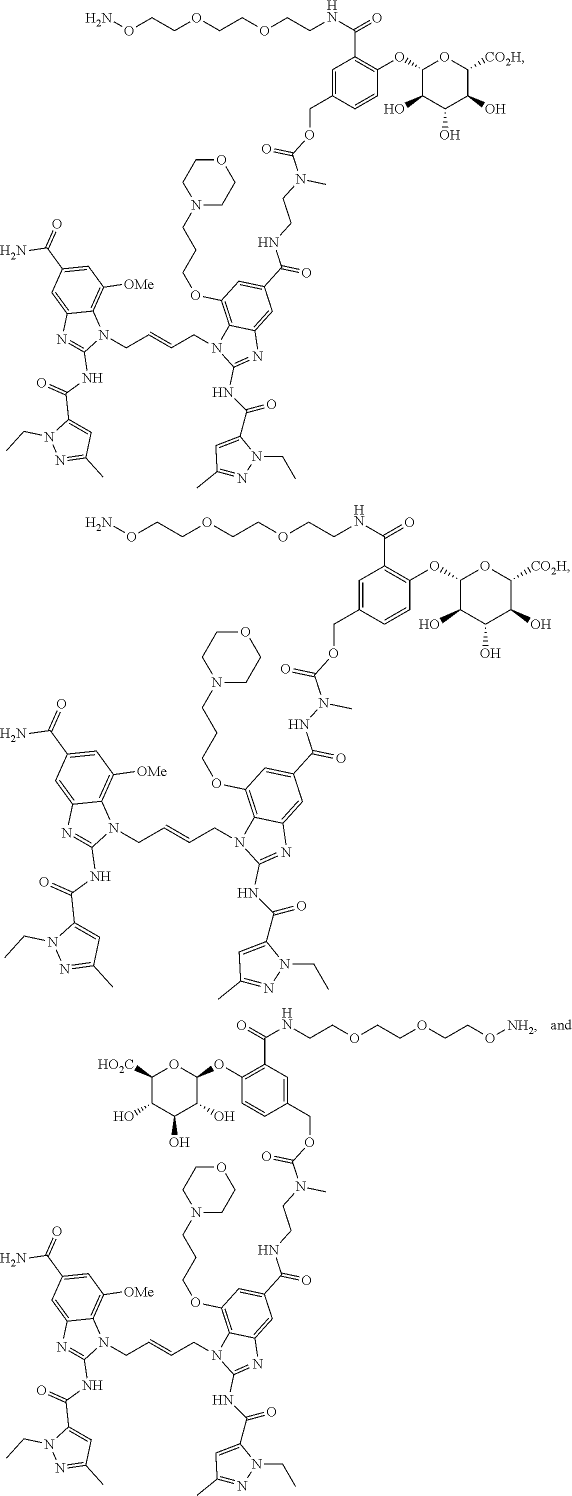

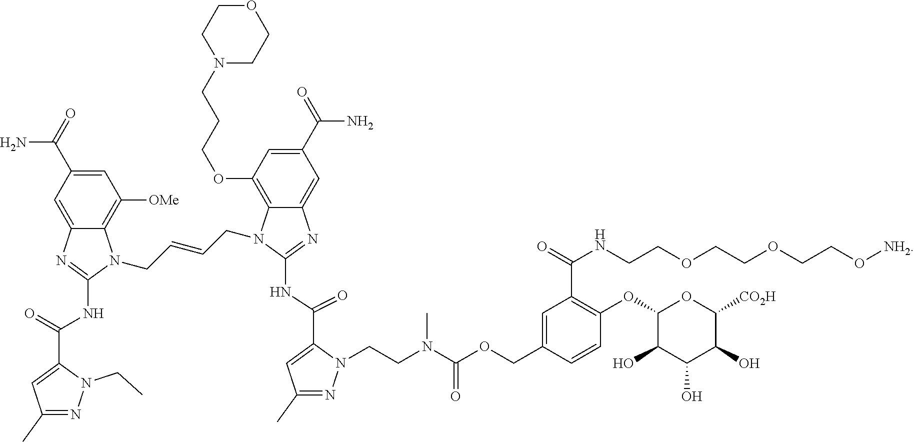 [SMILES: CCn1nc(C)cc1C(=O)Nc1nc2cc(C(N)=O)cc(OC)c2n1C/C=C/Cn1c(NC(=O)c2cc(C)nn2CCN(C)C(=O)OCc2ccc(O[C@@H]3O[C@H](C(=O)O)[C@@H](O)[C@H](O)[C@H]3O)c(C(=O)NCCOCCOCCON)c2)nc2cc(C(N)=O)cc(OCCCN3CCOCC3)c21]

In some embodiments, U is a saccharide. In some embodiments, the saccharide is a glucuronide. In some embodiments, the glucuronide is

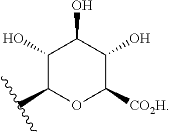 [SMILES: *[C@@H]1O[C@H](C(=O)O)[C@@H](O)[C@H](O)[C@H]1O]

In some embodiments, the compound is selected from:

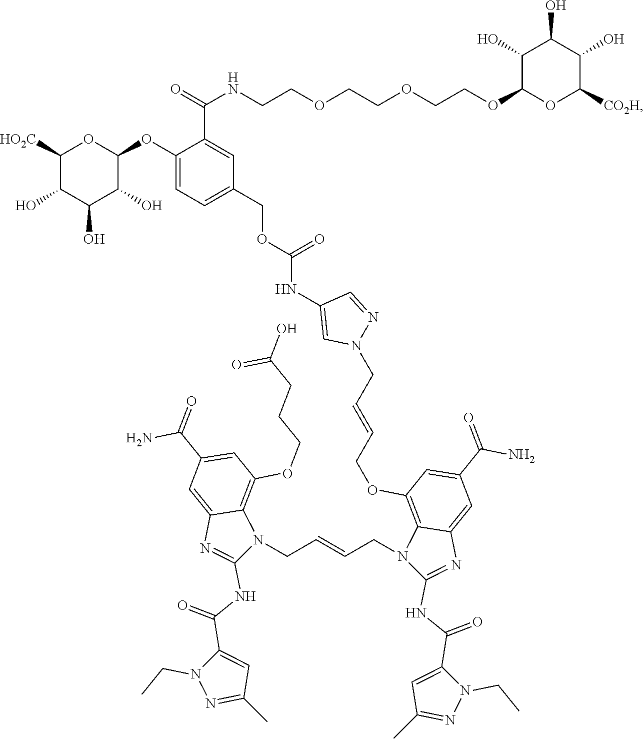 [SMILES: CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCO[C@@H]5O[C@H](C(=O)O)[C@@H](O)[C@H](O)[C@H]5O)c4)cn3)c2n1C/C=C/Cn1c(NC(=O)c2cc(C)nn2CC)nc2cc(C(N)=O)cc(OCCCC(=O)O)c21]

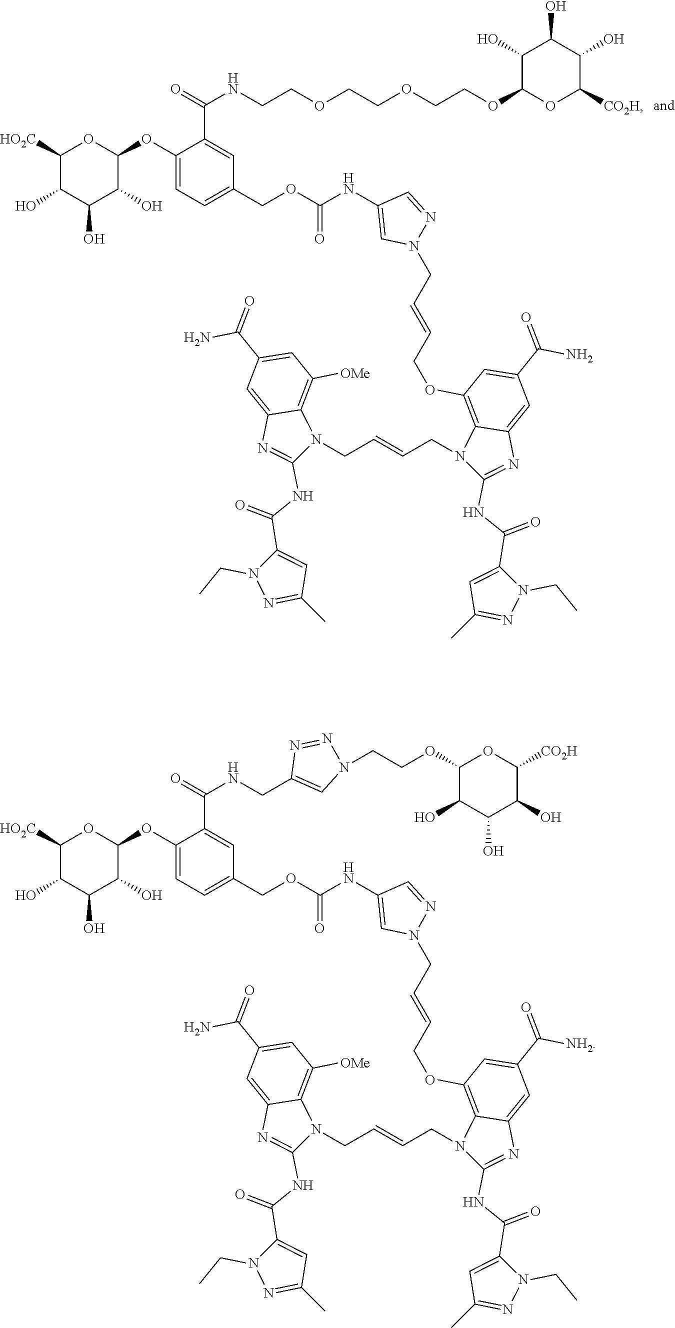 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCO[C@@H]5O[C@H](C(=O)O)[C@@H](O)[C@H](O)[C@H]5O)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCc5cn(CCO[C@@H]6O[C@H](C(=O)O)[C@@H](O)[C@H](O)[C@H]6O)nn5)c4)cn3)c21]

In some embodiments, T is selected from:

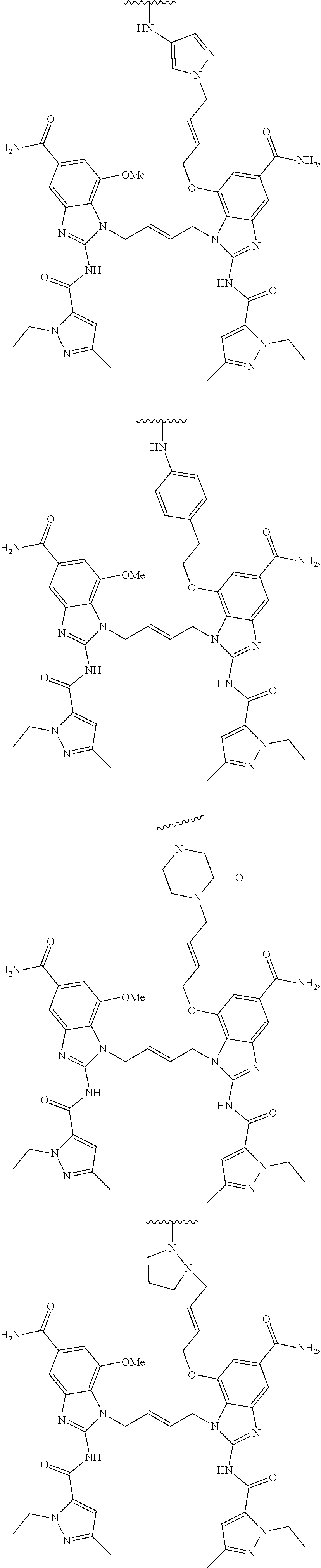

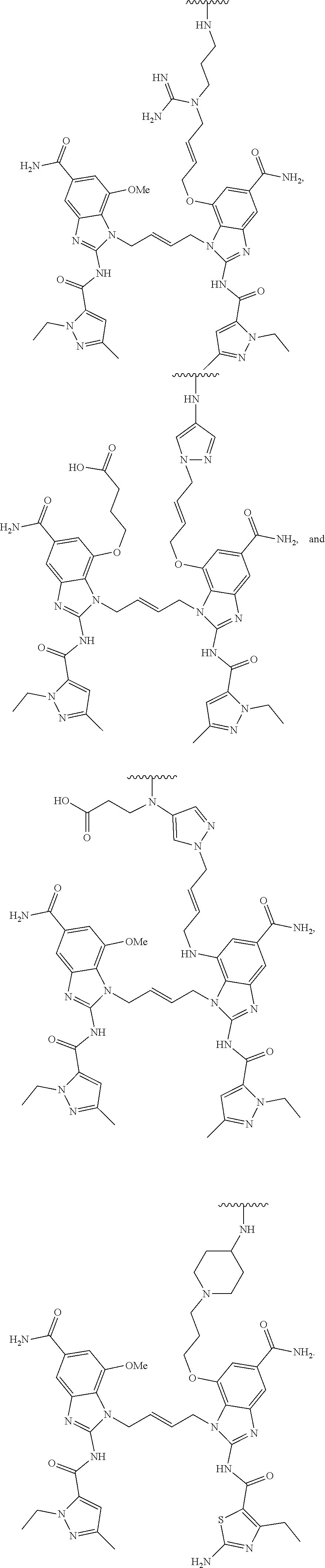

In some embodiments, Y is selected from;

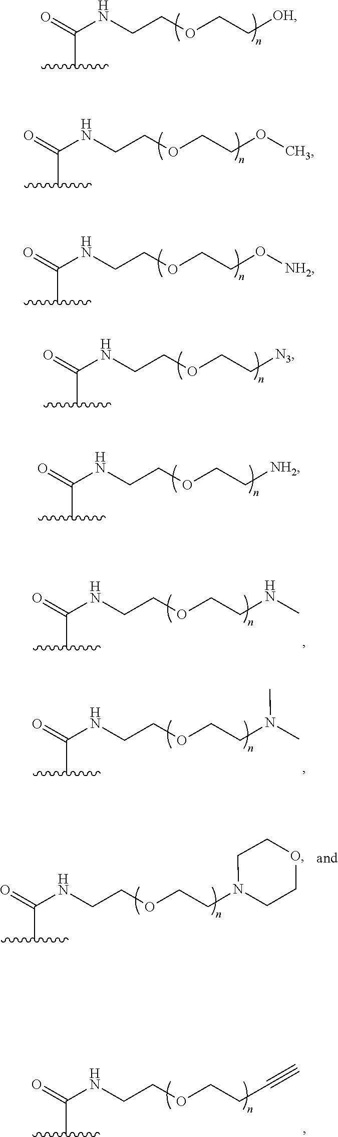

wherein n represents an integer from 1 to 15.

In some embodiments, Y is selected from:

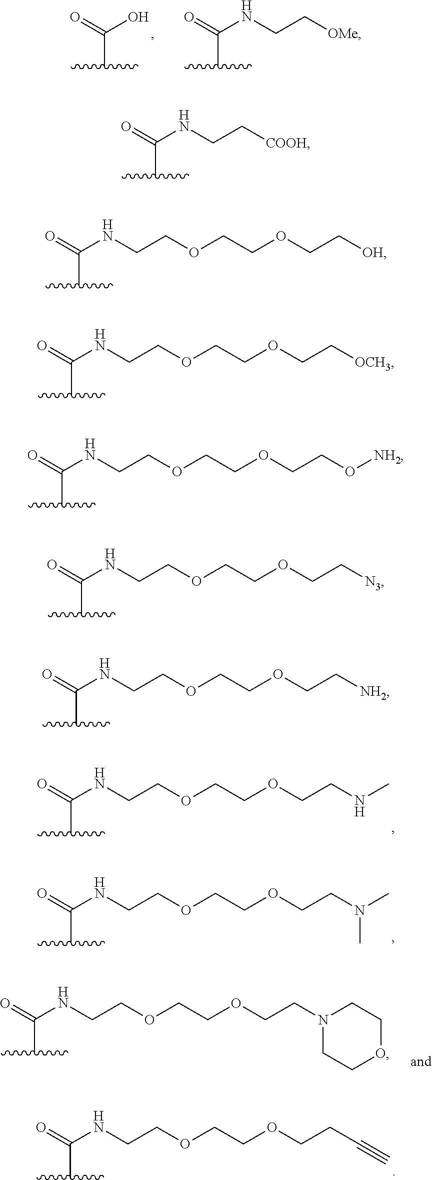

In some embodiments, the compound is selected from:

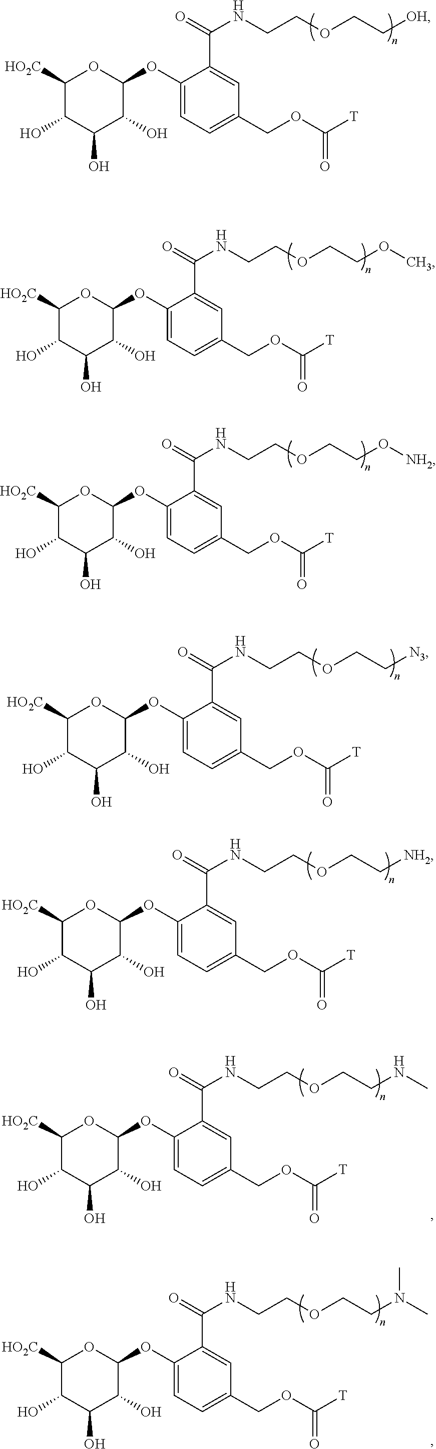

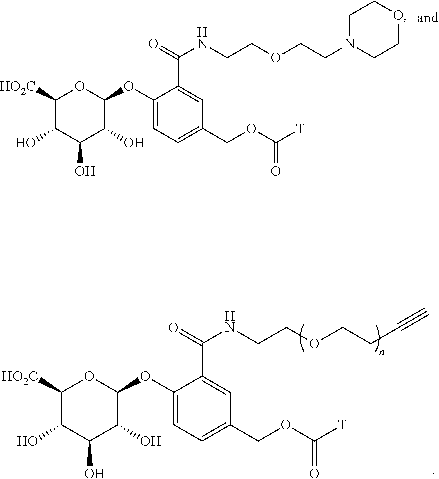

wherein n represents an integer from 1 to 15.

In some embodiments, the compound is selected from:

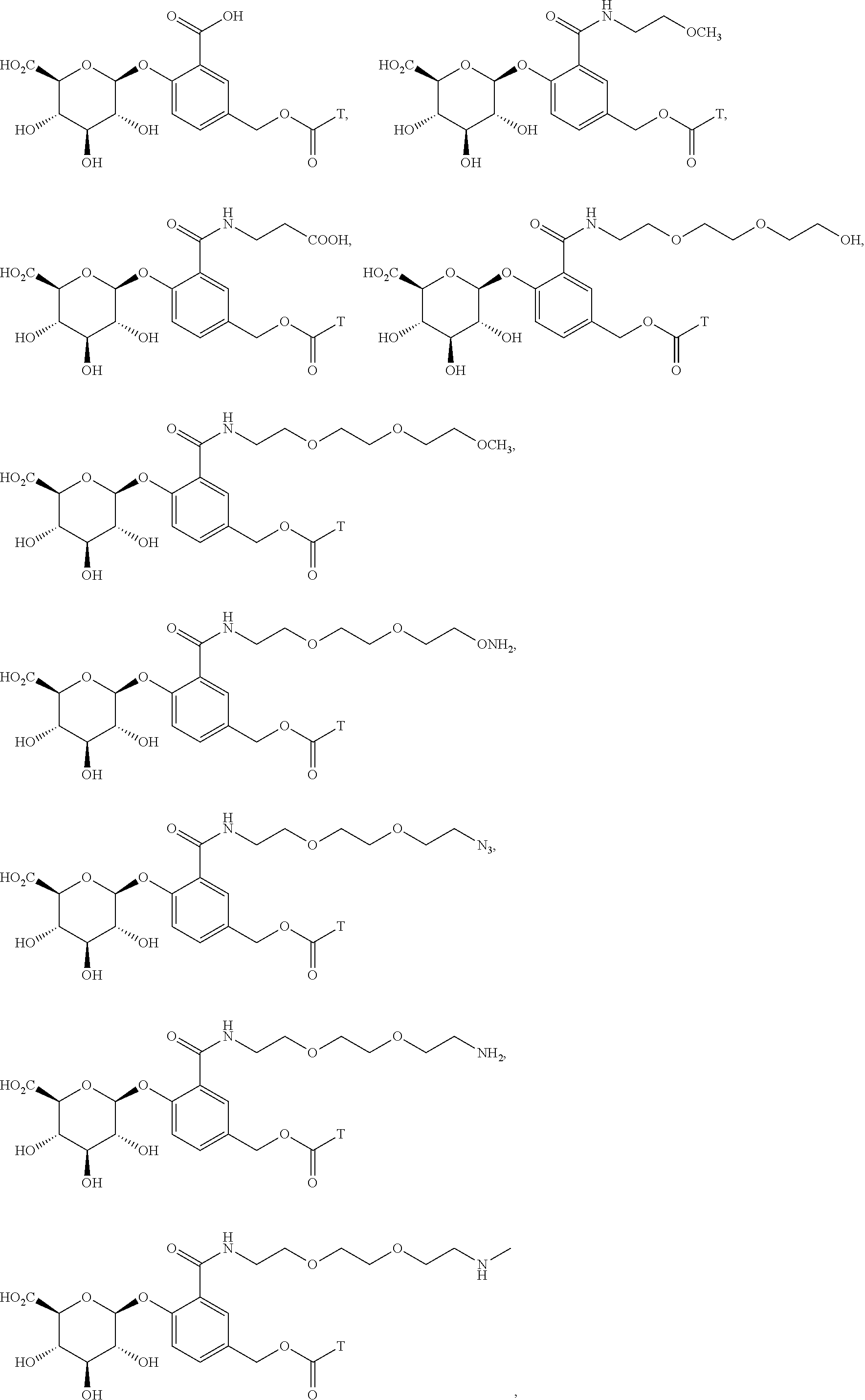

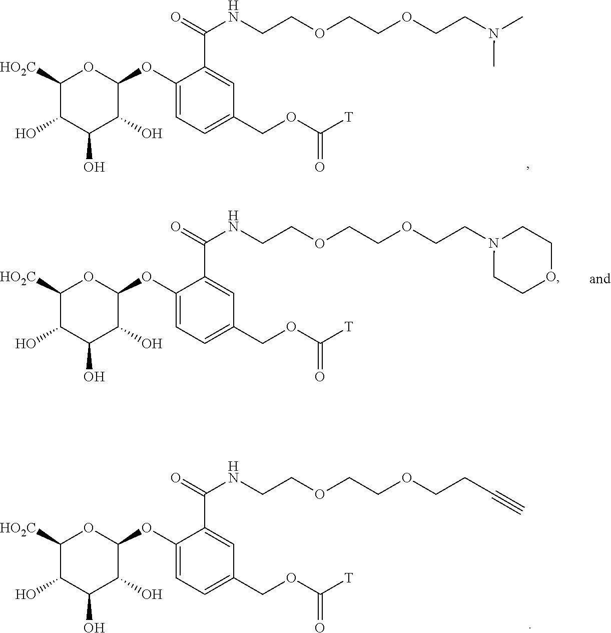

In some embodiments, the compound is selected from:

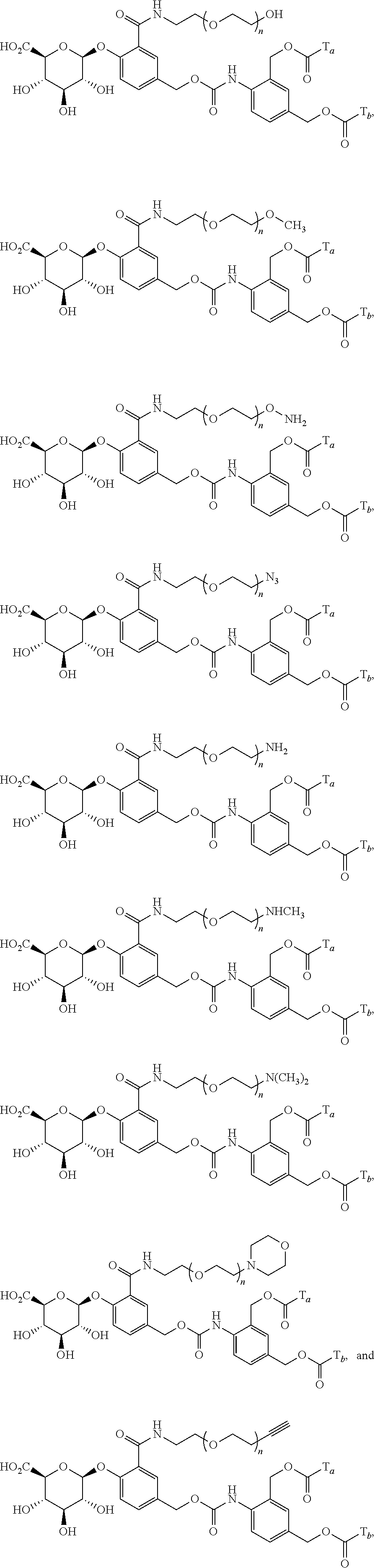

wherein n represents an integer from 1 to 15.

In some embodiments, the compound is selected from:

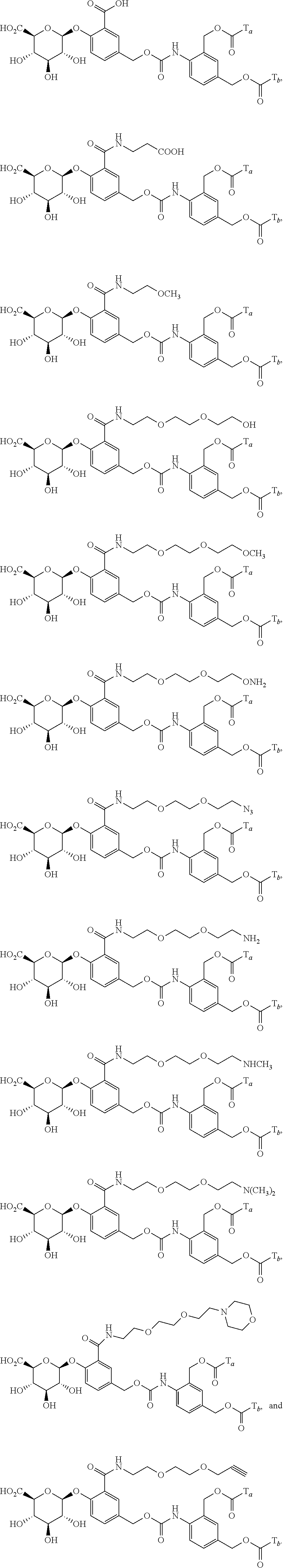

In some embodiments, the present disclosure relates to pharmaceutical compositions comprising a compound of the disclosure, such as a compound of formula (I) or a pharmaceutically acceptable salt thereof, and a pharmaceutically acceptable excipient.

In some embodiments, the present disclosure relates to a method of preventing or treating a disease mediated by stimulator of interferon genes (STING) in a subject in need thereof, the method comprising administering to the subject the compound of formula (I) or a pharmaceutically acceptable salt thereof or the pharmaceutical composition comprising the compound of formula (I) or a pharmaceutically acceptable salt thereof.

In some embodiments, the present disclosure relates to the use of the compound of formula (I) or a pharmaceutically acceptable salt thereof or the pharmaceutical composition comprising the compound of formula (I) or a pharmaceutically acceptable salt thereof for treating or preventing a disease mediated by stimulator of interferon genes (STING) in a subject in need thereof.

In some embodiments, the present disclosure relates to the compound of formula (I) or a pharmaceutically acceptable salt thereof or the pharmaceutical composition comprising the compound of formula (I) or a pharmaceutically acceptable salt thereof for use in treating or preventing a disease mediated by stimulator of interferon genes (STING) in a subject in need thereof.

In some embodiments, the disease mediated by STING is cancer.

In some embodiments, the cancer is selected from lung cancer, small cell lung cancer, gastrointestinal cancer, colorectal cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi sarcoma, and melanoma.

In some embodiments, the present disclosure relates to a method of inducing an immune response in a subject in need thereof, the method comprising administering to the subject the compound of formula (I) or a pharmaceutically acceptable salt thereof or the pharmaceutical composition comprising the compound of formula (I) or a pharmaceutically acceptable salt thereof.

In some embodiments, the present disclosure relates to the use of the compound of formula (I) or a pharmaceutically acceptable salt thereof or the pharmaceutical composition comprising the compound of formula (I) or a pharmaceutically acceptable salt thereof for inducing an immune response in a subject in need thereof.

In some embodiments, the present disclosure relates to the compound of formula (I) or a pharmaceutically acceptable salt thereof or the pharmaceutical composition comprising the compound of formula (I) or a pharmaceutically acceptable salt thereof for use in inducing an immune response in a subject in need thereof.

In some embodiments, the inducing of the immune response is effective to prevent or treat a disease mediated by STING in the subject.

In some embodiments, the disease mediated by STING is cancer.

In some embodiments, the cancer is selected from lung cancer, small cell lung cancer, gastrointestinal cancer, colorectal cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi sarcoma, and melanoma.

In some embodiments, the present disclosure relates to a method of modulating the activity of a STING adaptor protein comprising contacting the STING adaptor protein with the compound of Formula (I) or a pharmaceutically acceptable salt thereof.

In some embodiments, the compound increases the activity of the STING adaptor protein.

In some embodiments, the STING-mediated disease is cancer, bacterial infection disease, viral infection disease, fungal infection disease, immune-mediated disorder, central nervous system disease, peripheral nervous system disease, neurodegenerative disease, cerebrovascular disease, peripheral Arterial disease, cardiovascular disease, allergic disease or inflammation. In specific embodiments, the STING-mediated disease is cancer or an infectious disease.

In some embodiments, the cancer is lung cancer, small cell lung cancer, gastrointestinal cancer, colorectal cancer, bowel cancer, breast cancer, ovarian cancer, prostate cancer, testicular cancer, liver cancer, kidney cancer, bladder cancer, pancreatic cancer, brain cancer, sarcoma, and osteosarcoma, Kaposi's sarcoma, and melanoma.

In some embodiments, the pharmaceutical composition may additionally contain a pharmaceutically effective amount of a chemotherapeutic agent.

In some embodiments, the pharmaceutical composition may include one or more therapeutic co-agents; and a pharmaceutically acceptable excipient may be additionally included.

In some embodiments, the therapeutic co-agent is an agent that exhibits a preventive, ameliorative, or therapeutic effect on STING-mediated diseases, or an agent that can reduce the expression of side effects that appear when administering a therapeutic agent for STING-mediated diseases, or it may be, but is not limited to, an agent that exhibits an immunity enhancing effect, and when applied in the form of a formulation together with the STING agent represented by the compound, it exhibits a therapeutically useful effect and/or improves the stability of the proteolytic agent, and/or reduce the side effects that may appear when administering the STING agonist represented by the compound, and/or, any agent that exhibits the effect of maximizing the therapeutic effect through the enhancement of immunity can be applied in combination.

Combinable therapeutic co-agents include, for example, auristatin, bexarotene, bicalutamide, BMS 184476, bleomycin, semadotin, chlorambucil, cyclophosphamide, docetaxol, docetaxel, carr boplatin, carmustine, cisplatin, cryptophycin, decitabine, dolastatin, doxorubicin, mibobulin isethionate, rhizoxin, sertenef, streptozocin, mitomycin, methotrexate, taxanes, nilutamide, nivolumab, Onapristone, paclitaxel, procarbazine, tamoxifen, tasonermine, tretinoin, vinblastine, vincristine, PD-1 antagonists, CTLA-4 antagonists, B7 co-stimulatory molecules, interleukin-2, interleukin-7, etc.

In some embodiments, the cancer is lung cancer, small cell lung cancer, gastrointestinal cancer, colorectal cancer, bowel cancer, breast cancer, ovarian cancer, prostate cancer, testicular cancer, liver cancer, kidney cancer, bladder cancer, pancreatic cancer, brain cancer, sarcoma, osteosarcoma, It may be selected from the group consisting of Kaposi's sarcoma and melanoma, but any carcinoma for which the STING agonist represented by the compound can exhibit a therapeutic effect can be applied. In some embodiments, there is provided a method for preventing or treating STING-mediated diseases, comprising administering the pharmaceutical composition to a patient. In addition, the compound of Formula 1 of the present invention can be used as an adjuvant for the treatment of other infectious diseases, diseases or disorders including cancer, in any one of its tautomers, stereoisomers and pharmaceutically usable salts.

### Combination Therapy

In some embodiments, the compound, an optical isomer thereof, a stereoisomer thereof, a solvate thereof, a tautomer thereof or a pharmaceutically acceptable salt thereof, or a pharmaceutical composition comprising them may be used alone or in one or more additional therapies (e.g., drug treatment or treatment) can be used in combination.

Combination therapy may, for example, combine two therapies or may combine three therapies (e.g., a triple therapy of three therapeutic agents) or more. The dosage of one or more of the additional therapies (e.g., non-drug treatments or therapeutics) may be reduced from the standard dosage when administered alone.

In some embodiments, the compound, an optical isomer thereof, a stereoisomer thereof, a solvate thereof, a tautomer thereof, or a pharmaceutically acceptable salt thereof, or a pharmaceutical composition comprising the same is administered before, after, or concurrently with one or more such additional therapies.

In some embodiments, the compound can be administered together, e.g, in a single pharmaceutical composition, or can be administered separately, and when administered separately, this can occur simultaneously or sequentially. Such sequential administration may be close in time or distant.

In some embodiments, the compound, the additional therapy may be administration of a side effect limiting agent. Examples of such side effect limiting agents are agents that can be used to treat nausea, and may include dronabinol, granisetron, metoclopramide, ondansetron and prochlorperazine, or pharmaceutically acceptable salts thereof.

In some embodiments, the compound, the additional therapy includes non-drug therapy (eg, surgery or radiation therapy). Examples of non-drug treatments include, but are not limited to, radiation therapy, cryotherapy, hyperthermia, surgery (eg, surgical resection of tumor tissue), and adoptive T cell transfer (ACT) therapy.

In some embodiments, combinable therapeutic agents may be compounds used in the treatment of cancer or conditions associated therewith, and suitable steroids include 21-acetoxy pregnenolone, alcomethasone, algestone, amcinonide, beclomethasone, betamethasone, desonide, chloroprednisone, clobetasol, clocortolone, cloprednol, corticosterone, cortisone, cortibazole, deflazacort, desonide, desoximethasone, dexamethasone, diflorasone, di flucortolone, difuprednate, enoxolone, fluazacort, fluchloronide, flumethasone, flunisolide, fluocinolone acetonide, fluocinonide, fluocortin butyl, fluocortolone, fluorometholone, Fluperolone Acetate, Fluprednidene Acetate, Fluprednisolone, Flurandrenolide, Fluticasone Propionate, Formocortal, Halocinonide, Halobetasol Propionate, Halomethasone, Hydrocortisone, Rote Prednol etabonate, mazipredone, medrisone, meprednisone, methylprednisolone, mometasone furoate, paramethasone, predcarbate, prednisolone, prednisolone 25-diethylaminoacetate, prednisolone sodium phosphate, prednisone, prednival, prednylidene, rimexolone, thixocortol, triamcinolone, triamcinolone acetonide, triamcinolone venetonide, triamcinolone hexacetonide and salts or derivatives thereof, but are not limited thereto.

In some embodiments, it may be a biological agent (e.g., a cytokine (e.g., an interferon or an interleukin such as IL-2)) used in the treatment of cancer or a condition associated therewith. In some embodiments, the biologic is an immunoglobulin-based biologic, such as a monoclonal antibody (e.g., a humanized antibody, fully human antibody, Fc fusion protein or functional fragment thereof). Antibody-drug conjugates are also included.

In some embodiments, the immune checkpoint inhibitor can be a monospecific antibody such as a monoclonal antibody, a fusion protein such as an Fc-receptor fusion protein, more specifically an inhibitor of CTLA-4 (eg an inhibitory antibody or small molecule inhibitor) (eg, an anti-CTLA-4 antibody or fusion protein), an inhibitor or antagonist of PD-1 (eg, an inhibitory antibody or small molecule inhibitor), an inhibitor or antagonist of PDL-1 (eg, an inhibitory antibody or small molecule inhibitor), PDL-2 inhibitors or antagonists (eg, inhibitory antibodies or Fc fusions or small molecule inhibitors) (eg, PDL-2/Ig fusion proteins) and the like, but are not limited thereto.

In some embodiments, the anti-cancer agent can be, for example, a chemotherapeutic agent or a targeted therapy. Specifically, anticancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogues, pyrimidine analogues, purine analogues and related inhibitors., vinca alkaloids, epipodophyllotoxins, antibiotics, L-asparaginase, topoisomerase inhibitors, interferons, platinum coordination complexes, anthracenedione substituted ureas, methylhydrazine derivatives, adrenocortical inhibitors, adrenocorticosteroids, progestins, Includes estrogens, antiestrogens, androgens, antiandrogens and gonadotropin analogues. Additional anti-cancer agents include leucovorin (LV), irenotecan, oxaliplatin, capecitabine, paclitaxel, docetaxel, ALK inhibitor receptor tyrosine kinase (RTK)/growth factor receptor (e.g. inhibitors of downstream members of SHP2 inhibitors, SOS1) inhibitors, Raf inhibitors, MEK inhibitors, ERK inhibitors, PI3K inhibitors, PTEN inhibitors, AKT inhibitors, or mTOR inhibitors, Ras inhibitors, Ras vaccines, inhibitors of the MAP kinase (MAPK) pathway (or “MAPK inhibitors”), RAS-RAF-Disrupters or inhibitors of the ERK or PI3K-AKT-TOR or PI3K-AKT signaling pathway, PD-1 or PD-L1 antagonists, immune modulating imides (ImiDs), GITR agonists, genetically engineered T-cells (such as, CAR-T cells), bispecific antibodies (e.g., BiTE), anti-PD-1, anti-PD-L1, anti-CTLA4, anti-LAG-3 and anti-OX40 agents, EGFR inhibitors, IGF-1R inhibitors; and the like, but are not limited thereto.

In some embodiments, anti-angiogenic agents include, but are not limited to, in vitro synthetically prepared chemical compositions, antibodies, antigen binding regions, radionuclides, and combinations and conjugates thereof.

In some embodiments, the autophagy inhibitor is chloroquine, 3-methyladenine, hydroxy chloroquine (Plaquenil™), bafilomycin A1, 5-amino-4-imidazole carboxamide riboside (AICAR), okadaic acid, type autophagy-inhibiting algal toxins that inhibit protein phosphatases of type 2A or type 1, cAMP, and drugs that elevate cAMP levels, such as adenosine, LY204002, N6-mercaptopurine riboside, and vinblastine, but are not limited thereto.

The compounds according to the present disclosure may be used in combination with the agents described herein or other suitable agents depending on the condition being treated. Thus, in some embodiments one or more compounds of the present disclosure will be co-administered with other therapies as described herein. When used in combination therapy, the compounds described herein may be administered simultaneously or separately with the second agent. Such combined administration can include simultaneous administration of both agents in the same dosage form, simultaneous administration in separate dosage forms, and separate administration. That is, a compound described herein and any agent described herein may be formulated together in the same dosage form and administered simultaneously. Alternatively, a compound of the present invention and any therapy described herein may be administered simultaneously, wherein the two agents are in separate preparations. In another alternative, a compound of the present disclosure may be administered followed by any therapy described herein, or vice versa. In some embodiments of separate administration protocols, the compounds of the present invention and any of the therapies described herein may be administered minutes apart, or hours apart, or days apart.

In some embodiments of any of the methods described herein, the first therapy (e.g., a compound of the invention) and one or more additional therapies may be administered in any order, simultaneously or sequentially.

The compound described above in the pharmaceutical composition of the present invention is contained in a therapeutically effective amount or a prophylactically effective amount. The preferred dosage of the compound according to the present invention varies depending on the condition and weight of the patient, the severity of the disease, the type of drug, the route and duration of administration, but can be appropriately selected by those skilled in the art. However, for a desirable effect, the compound of Formula (I) of the present invention may be administered in an amount of 0.0001 to 1000 mg/kg, preferably 0.01 to 500 mg/kg, divided into once to several times a day.

In the composition described above, the compound of Formula (I) may be blended in an amount of 0.0001 to 50% by weight based on the total weight of the total composition.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4^{th} ed.”, W. H. Freeman & Co., New York (2000); Griffiths et al., “Introduction to Genetic Analysis, 7^{th} ed.”, W. H. Freeman & Co., N.Y. (1999); and Gilbert et al., “Developmental Biology, 6^{th} ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituent and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “alkyl” used herein refers to a linear or branched saturated monovalent hydrocarbon. For example, an alkyl group may have 1 to 10 carbon atoms (that is, (C₁₋₁₀)alkyl) or 1 to 8 carbon atoms (that is, (C₁₋₈)alkyl) or 1 to 6 carbon atoms (that is, (C₁₋₆ alkyl) or 1 to 4 carbon atoms (that is, (C₁₋₄)alkyl). Examples of the alkyl group include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃), but are not limited thereto.

Furthermore, the term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer. In certain embodiments, alkyl is unsubstituted, except as otherwise specified. However, if not specified, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “alkenyl” used herein refers to a linear or branched monovalent hydrocarbon radical having at least one carbon-carbon double bond. For example, an alkenyl group may include 2 to 8 carbon atoms (that is, C₂₋₈ alkenyl), or 2 to 6 carbon atoms (that is, C₂₋₆ alkenyl), or 2 to 4 carbon atoms (that is, C₂₋₄ alkenyl). Examples of the alkenyl group are ethylene or vinyl (—CH═CH₂), allyl (—CH₂CH═CH₂), 5-hexenyl (—CH₂CH₂CH₂CH₂CH═CH₂), and 3-hexenyl (—CH₂CH₂CH═CHCH₂CH₂), and are not limited thereto. Throughout the present specification, one terminal hydrogen of the alkenyl group is omitted and may be connected with the next linking group. In certain embodiments, alkenyl is unsubstituted, except as otherwise specified.

The term “alkylene” used herein refers to a linear or branched divalent saturated hydrocarbon group having 1 to 6 (C₁₋₆) carbon atoms. For example, an alkylene having 1 to 4 (C₁₋₄) carbon atoms may be used. Examples thereof include, but are not limited to, methylene, ethylene, trimethylene (propylene), and tetramethylene (n-butylene).

The term “alkynyl” used herein refers to a linear or branched monovalent hydrocarbon radical having at least one carbon-carbon triple bond. For example, an alkynyl group may include 2 to 8 carbon atoms (that is, C₂₋₈ alkynyl), or 2 to 6 carbon atoms (that is, C₂₋₆ alkynyl), or 2 to 4 carbon atoms (that is, C₂₋₄ alkynyl). Examples of alkynyl groups are acetylenyl (—C≡CH), propargyl (—CH₂C≡CH), and —CH₂—C≡CH₃, but are not limited thereto. In certain embodiments, alkynyl is unsubstituted, except as otherwise specified.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆ alkyl group, for example, contains from one to six carbon atoms in the chain.

Affixing the suffix “ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

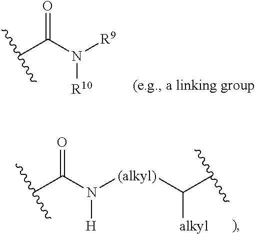

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

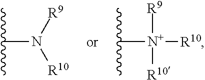

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl” or “arylalkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

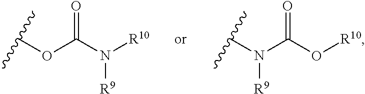

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In exemplary embodiments, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like. Non-limiting examples of monocyclic cycloalkyls are cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, and 1-cyclohex-3-enyl.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroarylalkyl”, “hetaralkyl”, and “heteroaralkyl”, as used herein, refers to an alkyl group substituted with a hetaryl group.

The term “heteroaryl” used herein refers to a single aromatic ring having at least one non-carbon atom in the ring, wherein the atom may be selected from oxygen, nitrogen, and sulfur, and “heteroaryl” may include a multiple condensed ring system having at least one such aromatic ring. The multiple condensed ring systems will be further described. Thus, the “heteroaryl” may include a single aromatic ring having about 1 to 6 carbon atoms and about 1-4 heteroatoms selected from oxygen, nitrogen and sulfur. Sulfur and nitrogen atoms may also exist in oxidized form, provided that the ring is aromatic. An example of the heteroaryl ring systems includes, but are not limited to, pyridyl, pyrimidinyl, oxazolyl, or furyl. In some embodiments, “heteroaryl” includes a multiple condensed ring system (for example, a ring system including 2, 3 or 4 rings), and the heteroaryl group as defined above may form a multiple condensed ring system through condensation with at least one ring selected from heteroaryl (used to form, for example, 1,8-naphthyridinyl), heterocycle (used to form, for example, 1,2,3,4-tetrahydro-1,8-naphthyridinyl), carbocycle (used to form, for example, 5,6,7,8-tetrahydroquinolyl), and aryl (used to form, for example, indazolyl). Thus, a heteroaryl (a single aromatic ring or a multiple condensed ring system) may have about 1-20 carbon atoms and about 1-6 heteroatoms in the heteroaryl ring. Such multiple condensed ring systems may be such that the carbocycle or heterocycle portion of the condensed ring may be substituted with one or more (for example, 1, 2, 3, or 4) oxo groups. The rings of a multiple condensed ring system may be linked to one another through fusion, spiro and cross-linking bonds as long as valency requirements are satisfied. The individual rings of the multiple condensed ring system may be linked to one another in any order. The point of attachment for the heteroaryl or the heteroaryl multiple condensed ring system may be any suitable atom of the heteroaryl or the heteroaryl multiple condensed ring system, including carbon atoms and heteroatoms (for example, nitrogen). Also, when a particular atom-range member heteroaryl (for example, (C₅-C₁₀) heteroaryl) is referred to, the atomic range is to be understood as being relative to the total number of ring atoms of the heteroaryl and as including a carbon atom and a heteroatom. For example, a C₅ heteroaryl may include a thiazolyl and a C₁₀ heteroaryl may include a quinolinyl. Examples of heteroaryls include pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole, and 3b, 4,4a,5-tetrahydro-1H-cyclopropa[3,4]cyclopenta[1,2-c]pyrazole, and are not limited thereto.

The term “heterocyclyl” or “heterocycle” used herein refers to a monosaturated or partially unsaturated non-aromatic compound or non-aromatic multi-ring system in which at least one heteroatom (that is, at least one cyclic heteroatom selected from oxygen, nitrogen and sulfur) is included in the ring. Unless otherwise specified, heterocyclyl groups have 5 to about 20 ring atoms, such as 3 to 12 ring atoms, such as 5 to 10 ring atoms. Thus, the term includes a single saturated or partially unsaturated ring (for example, 3, 4, 5, 6 or 7-membered rings), having about 1 to 6 cyclic carbon atoms and about 1 to 3 cyclic heteroatoms selected from oxygen, nitrogen and sulfur, in the ring. The rings of a multiple condensed ring system may be linked to one another through fusion, spiro and cross-linking bonds as long as valency requirements are satisfied. Examples of heterocycles include azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-chloropiperidine, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

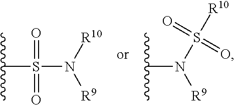

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

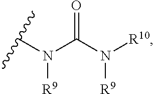

wherein R⁹ and R¹⁰ independently represent hydrogen or a hydrocarbyl.

The term “azido” is art-recognized and may be represented by the general formula

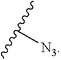

The term “hydrazido” is art-recognized and may be represented by the general formula

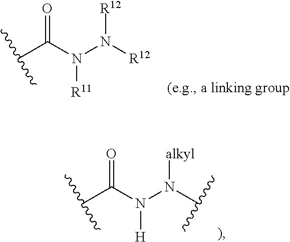

wherein R¹¹ and R¹² independently represent hydrogen or a hydrocarbyl.

The term “guanidino” is art-recognized and may be represented by the general formula

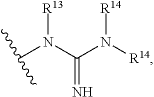

wherein R¹³ and R¹⁴ independently represent hydrogen or a hydrocarbyl.

The term “DBCO” used herein refers to an optionally substituted dibenzocyclooctyne moiety, e.g., the following structure:

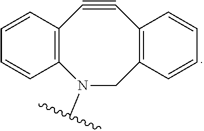 [SMILES: *N1Cc2ccccc2C#Cc2ccccc21]

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I.

Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

The term “cleavage group” refers to a chemical moiety which dissociates when subjected to a stimulus, such as acidic conditions, basic conditions, reducing conditions, oxidizing conditions, light, or heat, or an enzyme, such as an esterase.

### EXAMPLES

The invention now being generally described, it will be more readily understood by reference to the following examples which are included merely for purposes of illustration of certain aspects and embodiments of the present invention and are not intended to limit the invention.

### Preparation of Compounds

### Preparation Example 1: Preparation of Intermediate Compound 2

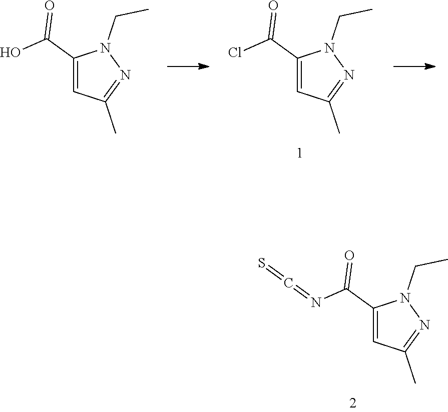

### Preparation of Intermediate Compound 1

After dissolving 1-ethyl-3-methyl-1H-pyrazole-5-carboxylic acid (8 g, 51.9 mmol) in dichloromethane (50 mL), oxalyl chloride (4.97 mL, 57.1 mmol) and N,N-dimethylformamide (0.1 mL) were added at 0° C., and the reaction solution was stirred at room temperature for 2 hours. The reaction solution was concentrated under reduced pressure to afford Intermediate Compound 1.

### Preparation of Intermediate Compound 2

After dissolving Intermediate Compound 1 (crude) in acetone (100 mL), potassium thiocyanate (6.5 g, 67.5 mmol) was added at 0° C. After stirring the reaction solution at room temperature for 30 minutes, hexane (100 mL) was added and the formed solid was filtered. The filtered solution was concentrated under reduced pressure and purified by column chromatography to afford Intermediate Compound 2 (9.7 g, 95%).

¹H-NMR (400 MHz, CDCl₃), δ 7.78 (d, 1H), 7.41 (s, 1H), 7.38 (d, 1H), 3.88 (s, 3H), 2.57 (s, 3H). EI-MS m/z: [M+H]⁺ 196.00.

### Preparation Example 2: Preparation of Intermediate Compound 5

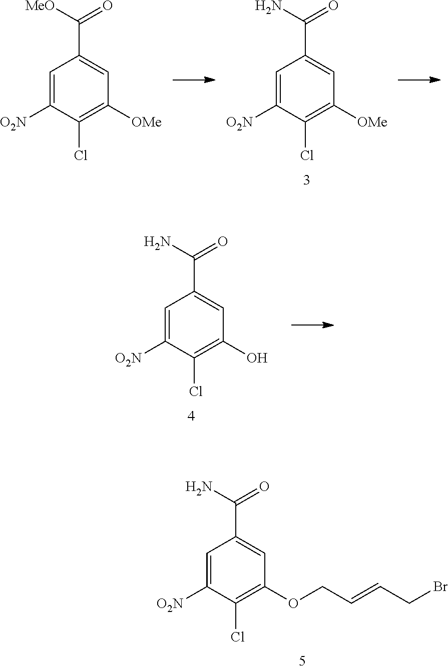 [SMILES: COC(=O)c1cc(OC)c(Cl)c([N+](=O)[O-])c1 ; COc1cc(C(N)=O)cc([N+](=O)[O-])c1Cl ; NC(=O)c1cc(O)c(Cl)c([N+](=O)[O-])c1 ; NC(=O)c1cc(OC/C=C/CBr)c(Cl)c([N+](=O)[O-])c1]

### Preparation of Intermediate Compound 3

To methyl 4-chloro-3-methoxy-5-nitrobenzoate (15 g, 61.1 mmol), aqueous ammonia solution (28˜30% ammonia, 200 mL) was added. The reaction solution was stirred at 50° C. for 6 hours, cooled to room temperature, and then washed with water, filtered, and lyophilized to afford Intermediate Compound 3 (9.51 g, 68%).

¹H-NMR (400 MHz, CDCl₃) δ 8.29 (s, 1H), 8.04 (d, 1H), 7.87 (d, 1H), 7.78 (s, 1H), 4.01 (s, 3H).

### Preparation of Intermediate Compound 4

Intermediate Compound 3 (300 mg, 1.30 mmol) was added to dichloromethane (9 mL), and then aluminum chloride (1.04 g, 7.81 mmol) was added at 0° C. The reaction solution was stirred for 21 hours at room temperature under nitrogen. The reaction solution was added to ice water, and then the resulting solid was filtered and lyophilized to afford Intermediate Compound 4 (223 mg, 79%).

¹H-NMR (400 MHz, CDCl₃), δ 11.73 (s, 1H), 8.21 (s, 1H), 7.92 (s, 1H), 7.80 (s, 1H), 7.66 (s, 1H).

### Preparation of Intermediate Compound 5

Intermediate Compound 4 (2 g, 9.23 mmol) and cesium carbonate (3.61 g, 11.08 mmol) were added to N,N-dimethylformamide (15 mL) at 0° C. under nitrogen and then stirred for 5 minutes. Trans-1,4-dibromo-2-butene (5.93 g, 27.70 mmol) was added to the reaction solution at room temperature under nitrogen and stirred for 2 hours. The resulting solution was extracted with ethyl acetate (20 mL×3) and washed with distilled water (15 mL×2) and brine (15 mL). The combined organic layers were dried over anhydrous sodium sulfate and filtered. The filtrate was concentrated. After solidification using dichloromethane and hexane, it was filtered and dried to afford Intermediate Compound 5 (2.53 g, 78%).

¹H-NMR (400 MHz, CDCl₃), δ 8.23 (s, 2H), 8.02 (d, J=1.8 Hz, 2H), 7.84 (d, J=1.8 Hz, 2H), 7.73 (s, 2H), 6.16-5.98 (m, 4H), 5.70 (s, 1H), 4.83 (d, J=3.5 Hz, 4H), 4.25 (d, J=5.4 Hz, 1H), 4.20-4.14 (m, 3H). EI-MS m/z: [M+H]⁺ 350.98.

### Preparation Example 3: Preparation of Intermediate Compound 9

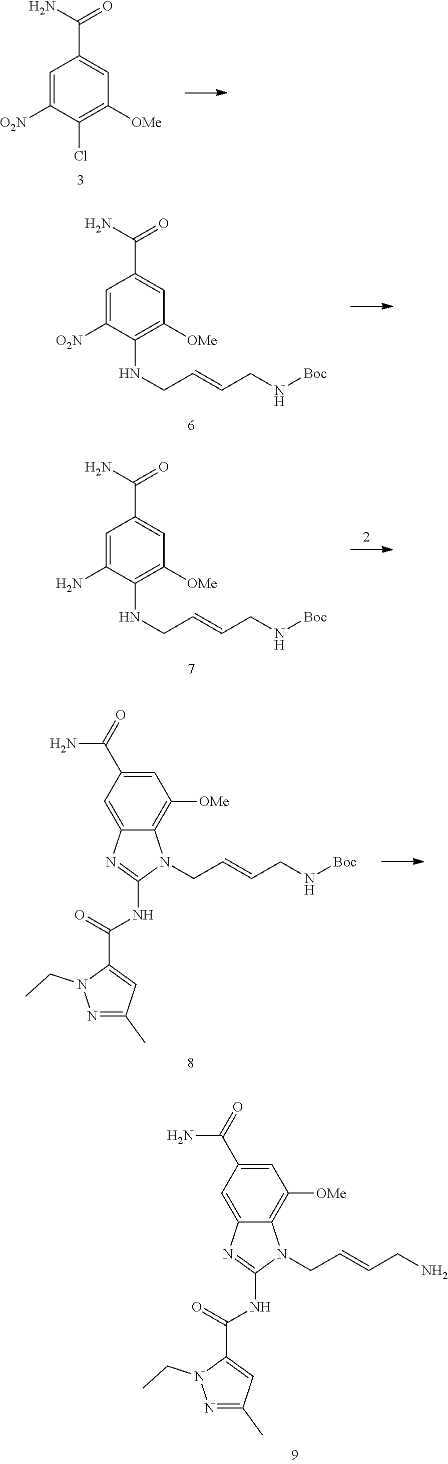 [SMILES: COc1cc(C(N)=O)cc([N+](=O)[O-])c1Cl ; COc1cc(C(N)=O)cc([N+](=O)[O-])c1NC/C=C/CNC(=O)OC(C)(C)C ; COc1cc(C(N)=O)cc(N)c1NC/C=C/CNC(=O)OC(C)(C)C ; CCn1nc(C)cc1C(=O)Nc1nc2cc(C(N)=O)cc(OC)c2n1C/C=C/CNC(=O)OC(C)(C)C ; CCn1nc(C)cc1C(=O)Nc1nc2cc(C(N)=O)cc(OC)c2n1C/C=C/CN]

### Preparation of Intermediate Compound 6

After dissolving 4-chloro-3-methoxy-5-nitro-benzamide (11.4 g, 61.57 mmol) in ethanol (100 mL), Intermediate Compound 3 (10 g, 43.4 mmol) and N,N-diisopropylethylamine (14.9 mL, 86.7 mmol) were added and stirred at 120° C. for 12 hours. The reaction solution was concentrated and diluted with diethyl ether (40 mL), and then the resulting solid was filtered and dried to afford Intermediate Compound 6 (12.6 g, 76%).

¹H-NMR (400 MHz, DMSO-d₆), δ 8.18 (d, 1H), 8.01 (s, 1H), 7.73 (t, 1H), 7.55 (d, 1H), 7.31 (s, 1H), 6.92 (s, 1H), 5.53 (s, 2H), 4.08 (s, 2H), 3.47 (s, 2H), 1.35 (m, 9H).

### Preparation of Intermediate Compound 7

After dissolving Intermediate Compound 6 (10 g, 26.28 mmol) in methanol (90 mL), ammonia aqueous solution (28-30% ammonia, 90 mL) and sodium hydrosulfite (Na₂S₂O₄, 45 g, 262.8 mmol) were sequentially added at 0° C., and the mixture was stirred at room temperature for 1 hour and 30 minutes. After adding methanol (100 mL) to the reaction solution, the resulting solid was filtered, the filtered solution was concentrated, and then diluted with dichloromethane (100 mL) and washed with distilled water (50 mL), and the organic layer was dried over anhydrous sodium sulfate. After filtration, it was concentrated under reduced pressure to afford Intermediate Compound 7 (6.9 g, 75%).

¹H-NMR (400 MHz, DMSO-d₆), δ 7.62 (br s, 1H), 6.98 (br s, 1H), 6.92 (t, 1H), 6.87 (d, 1H), 6.79 (d, 1H), 5.57 (q, 2H), 4.67 (br s, 2H), 3.82 (br s, 1H), 3.76 (s, 3H), 3.51 (dd, 4H), 1.37 (s, 9H).

### Preparation of Intermediate Compound 8

After dissolving Intermediate Compound 7 (6.9 g, 19.7 mmol) in N,N-dimethylformamide (50 mL), Intermediate Compound 2 (4.9 g, 25.59 mmol) was added at 0° C. and stirred at room temperature for 30 minutes. Triethylamine (5.4 mL, 39.38 mmol) and N-(3-dimethylaminopropyl)-N′-ethylcarbodiimide (4.2 g, 27.56 mmol) were added at 0° C., and the mixture was stirred at room temperature for 17 hours. After concentrating the reaction solution, it was diluted with diethyl ether (20 mL) and the resulting solid was filtered to afford Intermediate Compound 8 (7.7 g, 76%).

¹H-NMR (400 MHz, DMSO-d₆), δ 12.86 (s, 1H), 8.02 (s, 1H), 7.67 (s, 1H), 7.40 (m, 2H), 6.93 (m, 1H), 6.65 (s, 1H), 5.67 (m, 2H), 4.93 (d, 2H), 4.61 (q, 2H), 3.98 (s, 3H), 3.51 (m, 2H), 2.55 (m, 2H), 2.18 (s, 3H), 1.35 (t, 3H), 1.32 (s, 9H).

### Preparation of Intermediate Compound 9

After dissolving Intermediate Compound 8 (7.7 g, 15.05 mmol) in dichloromethane (25 mL) and methanol (25 mL), hydrochloric acid (4 M 1,4-dioxane solution, 27 mL) was added and stirred for 2 hours and 30 minutes. The reaction solution was concentrated, diluted with diethyl ether (20 mL), and then the resulting solid was filtered to afford Intermediate Compound 9 (5.6 g, 76%).

¹H-NMR (400 MHz, DMSO-d₆) δ 8.02 (s, 1H), 8.67 (d, 1H), 7.41 (d, 1H), 7.37 (s, 1H), 6.66 (s, 1H), 6.01 (m, 1H), 5.65 (m, 1H), 4.97 (d, 2H), 4.60 (q, 2H), 3.98 (s, 3H), 2.66 (m, 5H), 2.33 (m, 3H), 1.35 (m, 3H). EI-MS m/z. [M+H]⁺ 823.0.

### Example 1: Preparation of Intermediate Compound 13

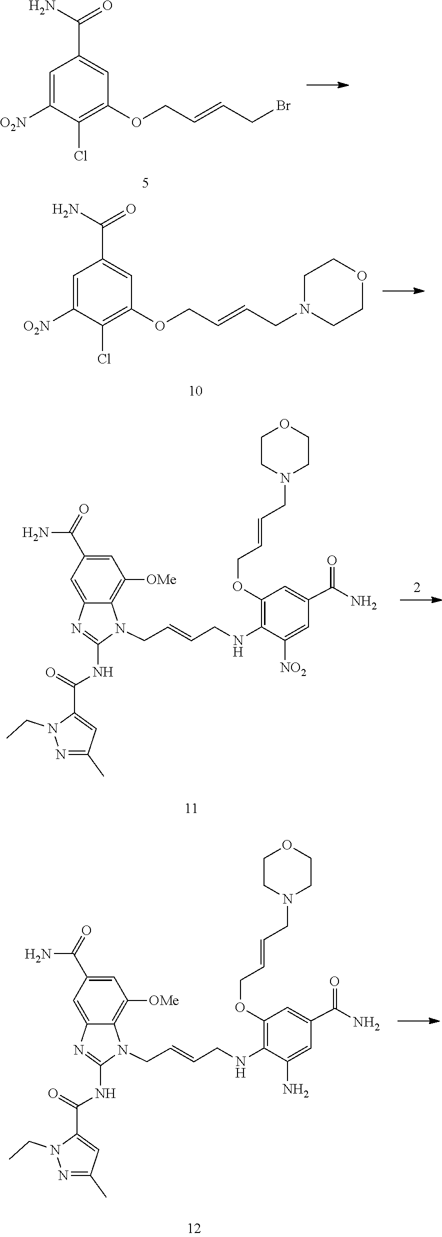 [SMILES: NC(=O)c1cc(OC/C=C/CBr)c(Cl)c([N+](=O)[O-])c1 ; NC(=O)c1cc(OC/C=C/CN2CCOCC2)c(Cl)c([N+](=O)[O-])c1 ; CCn1nc(C)cc1C(=O)Nc1nc2cc(C(N)=O)cc(OC)c2n1C/C=C/CNc1c(OC/C=C/CN2CCOCC2)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C/CN1CCOCC1]

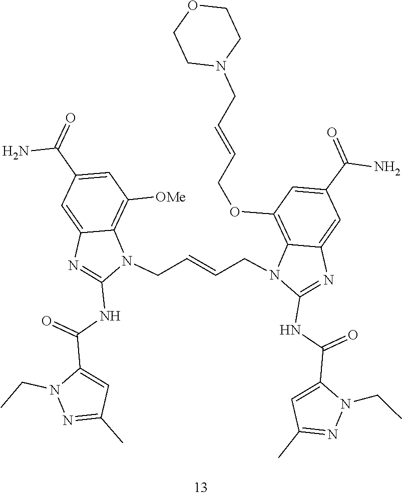 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCOCC3)c21]

### Preparation of Intermediate Compound 10

After dissolving Intermediate Compound 5 (580 mg, 1.66 mmol) in N,N-dimethylformamide (5 mL), morpholine (0.13 mL, 1.01 mmol) and cesium carbonate (590 mg, 1.81 mmol) were added under nitrogen. The reaction solution was stirred at room temperature for 2 hours, diluted with ethyl acetate (50 mL), washed with distilled water (50 mL×2), and dried with anhydrous sodium sulfate. After filtration, the mixture was concentrated under reduced pressure, diluted with dichloromethane and hexane, and the resulting solid was filtered and dried to afford Intermediate Compound 10 (504 mg, 70%).

¹H-NMR (400 MHz, CDCl₃), δ 7.73-7.66 (m, 1H), 6.03-5.84 (m, 1H), 4.75 (dd, J=5.0, 1.2 Hz, 1H), 3.75-3.68 (m, 2H), 3.06 (dd, J=6.0, 1.1 Hz, 1H), 2.46 (t, J=4.7 Hz, 2H). EI-MS m/z: [M+H]⁺ 356.09.

### Preparation of Intermediate Compound 11

After dissolving Intermediate Compound 9 (818 mg, 1.68 mmol) and Intermediate Compound 10 (300 mg, 0.84 mmol) in normal butyl alcohol (13 mL), N,N-diisopropylethylamine (0.74 mL, 4.21 mmol) was added at room temperature, heated to 120° C., and stirred for 24 hours. After cooling the reaction solution to room temperature, the reaction solution was diluted with dichloromethane (100 mL) and methanol (20 mL) and washed with distilled water (50 mL). The organic layers were combined, dried over anhydrous sodium sulfate and filtered. The filtrate was concentrated under reduced pressure. After dilution with diethyl ether, the resulting solid was filtered and dried to afford Intermediate Compound 11 (129 mg, 21%). EI-MS m/z: [M+H]⁺ 731.03.

### Preparation of Intermediate Compound 12

After dissolving Intermediate Compound 11 (129 mg, 0.17 mmol) in methyl alcohol (2 mL) and distilled water (0.1 mL), aqueous ammonia solution (28-30% ammonia, 0.17 mL) and sodium hydrosulfite (Na₂S₂O₄, 321 mg, 1.84 mmol) were added to the reaction solution under nitrogen. After stirring at room temperature for 1 hour, methanol (50 mL) was added to the reaction solution, and the resulting solid was filtered, and the filtered solution was concentrated, and then diluted with dichloromethane (100 mL), washed with distilled water (50 mL), and then the organic layer was dried with anhydrous sodium sulfate. After filtration, it was concentrated under reduced pressure to afford Intermediate Compound 12 (126 mg, crude). EI-MS m/z: [M+H]⁺ 701.08.

### Preparation of Intermediate Compound 13

After dissolving Intermediate Compound 12 (126 mg, 0.18 mmol, crude) in N,N-dimethylformamide (2 mL), Intermediate Compound 2 (39 mg, 0.2 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (40 mg, 0.21 mmol) and triethylamine (0.04 mL, 0.27 mmol) were added, and stirred at room temperature for 16 hours. The resulting material was concentrated under reduced pressure and purified by HPLC to afford Intermediate Compound 13 (5.6 mg, 4%). EI-MS m/z: [M+H]⁺ 862.03.

### Example 2: Preparation of Intermediate Compound 18

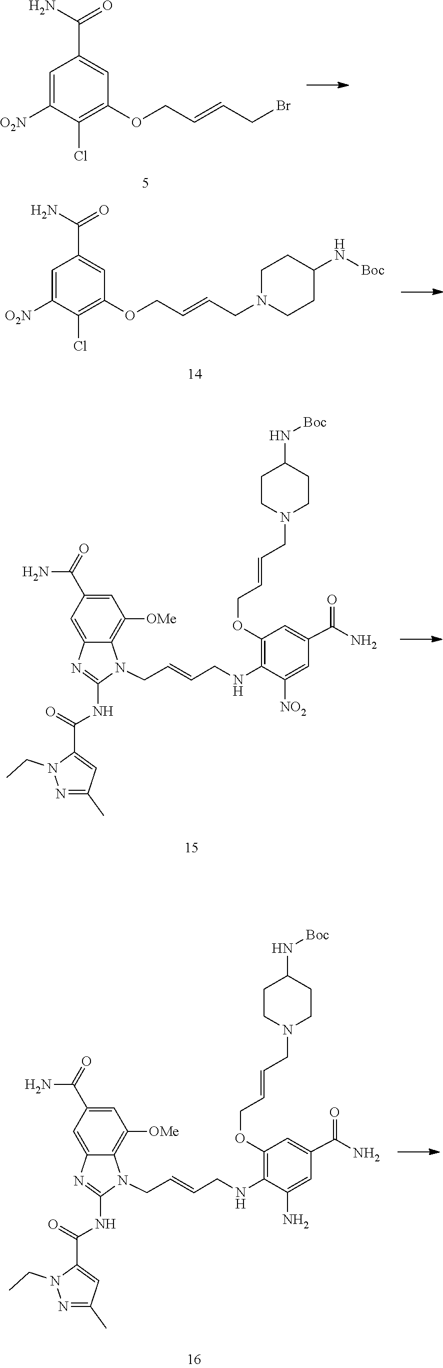 [SMILES: NC(=O)c1cc(OC/C=C/CBr)c(Cl)c([N+](=O)[O-])c1 ; CC(C)(C)OC(=O)NC1CCN(C/C=C/COc2cc(C(N)=O)cc([N+](=O)[O-])c2Cl)CC1 ; CCn1nc(C)cc1C(=O)Nc1nc2cc(C(N)=O)cc(OC)c2n1C/C=C/CNc1c(OC/C=C/CN2CCC(NC(=O)OC(C)(C)C)CC2)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C/CN1CCC(NC(=O)OC(C)(C)C)CC1]

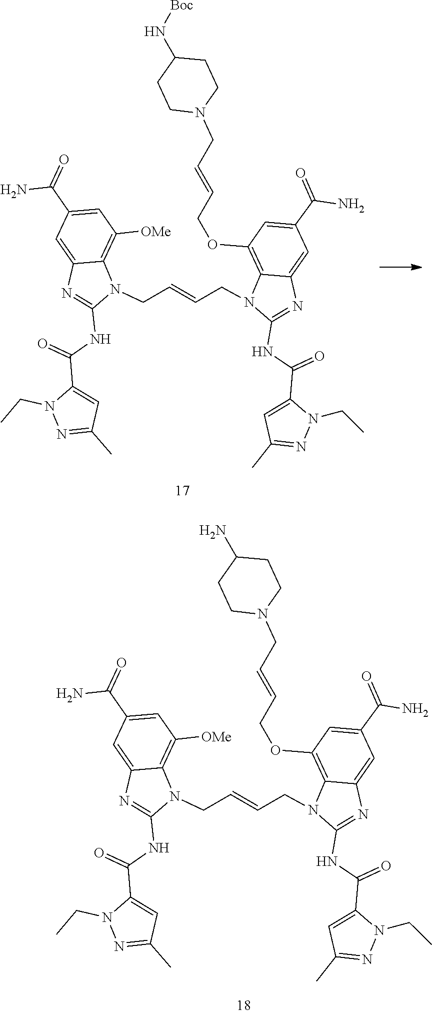

### Preparation of Intermediate Compound 14

After dissolving Intermediate Compound 5 (265 mg, 0.76 mmol) in N,N-dimethylformamide (4 mL), t-butyl piperidin-4-ylcarbamate (167 mg, 0.83 mmol) and cesium carbonate (296 mg, 0.91 mmol) were added under nitrogen. The reaction solution was stirred at room temperature for 2 hours, diluted with ethyl acetate (50 mL), washed with distilled water (50 mL×2), and dried with anhydrous sodium sulfate. After filtration, the mixture was concentrated under reduced pressure, diluted with dichloromethane and hexane, and the resulting solid was filtered and dried to afford Intermediate Compound 14 (248 mg, 70%). EI-MS m/z: [M+H]⁺ 469.06.

### Preparation of Intermediate Compound 15

After dissolving Intermediate Compound 9 (628 mg, 1.3 mmol) and Compound 14 (304 mg, 0.65 mmol) in normal butyl alcohol (5 mL), diisopropylethylamine (0.56 mL, 3.24 mmol) was added at room temperature, heated to 120° C. and stirred for 24 hours. After cooling the reaction solution to room temperature, the reaction solution was diluted with dichloromethane (100 mL) and methanol (20 mL) and washed with distilled water (50 mL). The reaction solution was dried with anhydrous sodium sulfate, filtered, concentrated under reduced pressure, diluted with dichloromethane and hexane, and the resulting solid was filtered and dried to afford Intermediate Compound 15 (124 mg, 23%). EI-MS m/z: [M+H]⁺ 844.02.

### Preparation of Intermediate Compound 16

After dissolving Intermediate Compound 15 (124 mg, 0.15 mmol) in methanol (4 mL) and distilled water (0.5 mL), an aqueous ammonia solution (28˜30% ammonia, 0.4 mL) and sodium hydrosulfite (Na₂S₂O₄, 218 mg, 2.94 mmol) were added to the reaction solution under nitrogen. The mixture was stirred at room temperature for 1 hour, diluted with methanol (50 mL), and filtered. The filtrate was concentrated under reduced pressure, diluted with dichloromethane (100 mL) and methanol (20 mL), and washed with distilled water (50 mL). The washed material was dried with anhydrous sodium sulfate and filtered. The filtrate was concentrated under reduced pressure to afford Intermediate Compound 16 (119 mg, crude). EI-MS m/z: [M+H]⁺ 814.81.

### Preparation of Intermediate Compound 17

After dissolving Intermediate Compound 16 (119 mg, 0.15 mmol, crude) in N,N-dimethylformamide (1 mL), Intermediate Compound 2 (32 mg, 0.16 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (35 mg, 0.18 mmol) and triethylamine (0.06 mL, 0.44 mmol) were added, and stirred at room temperature for 16 hours. The reaction solution was concentrated under reduced pressure and purified by column chromatography to afford Intermediate Compound 17 (36 mg, 25%). EI-MS m/z: [M+H]⁺ 975.15.

### Preparation of Intermediate Compound 18

After dissolving Intermediate Compound 17 (36 mg) in dichloromethane (1 mL), trifluoroacetic acid (1 mL) was added at 0° C. under nitrogen. The reaction solution was stirred at room temperature for 2 hours, concentrated, and then purified by HPLC to afford Intermediate Compound 18 (15.2 mg, 47%). EI-MS m/z: [M+H]⁺ 875.06.

### Example 3: Preparation of Intermediate Compound 23

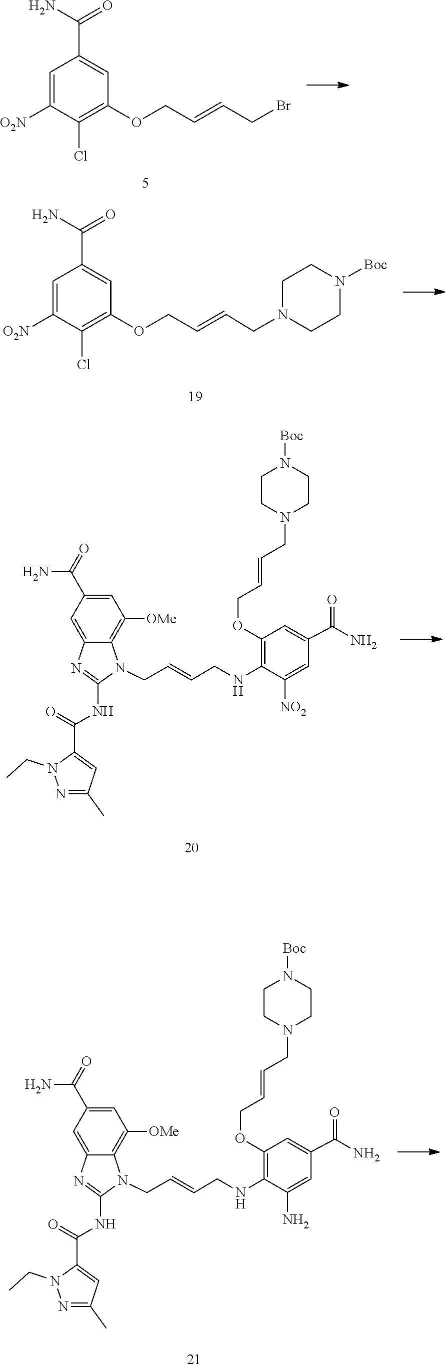 [SMILES: NC(=O)c1cc(OC/C=C/CBr)c(Cl)c([N+](=O)[O-])c1 ; CC(C)(C)OC(=O)N1CCN(C/C=C/COc2cc(C(N)=O)cc([N+](=O)[O-])c2Cl)CC1 ; CCn1nc(C)cc1C(=O)Nc1nc2cc(C(N)=O)cc(OC)c2n1C/C=C/CNc1c(OC/C=C/CN2CCN(C(=O)OC(C)(C)C)CC2)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C/CN1CCN(C(=O)OC(C)(C)C)CC1]

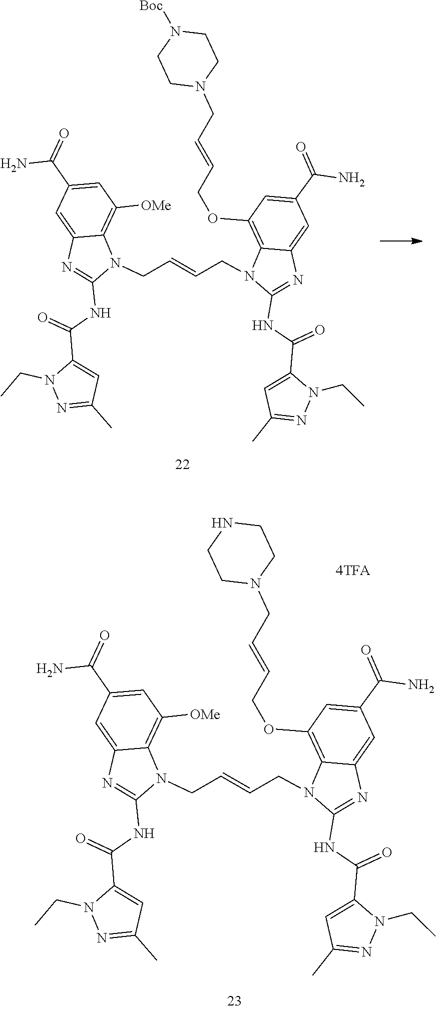

### Preparation of Intermediate Compound 19

After dissolving Intermediate Compound 5 (500 mg, 1.43 mmol) in N,N-dimethylformamide (5 mL), t-butyl piperazine-1-carboxylate (320 mg, 1.71 mmol) and cesium carbonate (512 mg, 1.57 mmol) were added under nitrogen. The reaction solution was stirred at room temperature for 2 hours, diluted with ethyl acetate (50 mL), washed with distilled water (50 mL×2), and dried with anhydrous sodium sulfate. The reaction solution was filtered, concentrated under reduced pressure, diluted with dichloromethane and hexane, and the resulting solid was filtered and dried to afford Intermediate Compound 19 (472 mg, 72%).

¹H-NMR (400 MHz, DMSO-d₆), δ 8.26 (s, 1H), 8.06 (s, 1H), 7.88 (s, 1H), 7.78 (s, 1H), 5.91-5.83 (m, 2H), 4.84 (d, 1H), 3.29-3.27 (m, 4H), 2.98 (d, 2H), 2.73 (t, 4H), 1.39 (s, 9H). EI-MS m/z: [M+H]⁺ 455.11.

### Preparation of Intermediate Compound 20

After dissolving Intermediate Compound 9 (495 mg, 1.02 mmol) and Intermediate Compound 19 (310 mg, 0.68 mmol) in normal butyl alcohol (7 mL), diisopropylethylamine (0.59 mL, 3.41 mmol) was added at room temperature, heated to 120° C. and stirred for 24 hours. The reaction solution was cooled to room temperature, diluted with dichloromethane (100 mL) and methanol (20 mL), and washed with distilled water (50 mL). The organic layer was dried with anhydrous sodium sulfate, filtered, and then concentrated under reduced pressure. The solid obtained by diluting with dichloromethane and diethyl ether was filtered and dried to afford Intermediate Compound 20 (336 mg, 46%). EI-MS m/z: [M+H]⁺ 830.01.

### Preparation of Intermediate Compound 21

After dissolving Intermediate Compound 20 (336 mg, 0.31 mmol) in methanol (10 mL) and distilled water (1 mL), an aqueous ammonia solution (28-30% ammonia, 0.33 mL) and sodium hydrosulfite (Na₂S₂O₄, 549 mg, 3.16 mmol) were added to the reaction solution under nitrogen. The mixture was stirred at room temperature for 1 hour, diluted with methanol (50 mL), and then filtered. The filtrate was concentrated under reduced pressure, diluted with dichloromethane (100 mL) and methanol (20 mL), and then washed with distilled water (50 mL). The organic layer was dried with anhydrous sodium sulfate, filtered, and then concentrated under reduced pressure to afford Intermediate Compound 21 (153 mg, 60%, crude). EI-MS m/z: [M+H]⁺ 800.09.

### Preparation of Intermediate Compound 22

After dissolving Intermediate Compound 21 (153 mg, 0.19 mmol, crude) in N,N-dimethylformamide (2 mL), Intermediate Compound 2 (45 mg, 0.23 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (192 mg, 0.28 mmol) and triethylamine (0.1 mL, 0.76 mmol) were added, and stirred at room temperature for 13 hours. The reaction solution was concentrated under reduced pressure and purified by column chromatography to afford an Intermediate Compound 22 (116 mg, 63%). EI-MS m/z: [M+H]⁺ 961.07.

### Preparation of Intermediate Compound 23

After dissolving Intermediate Compound 22 (41 mg) in dichloromethane (5 mL), trifluoroacetic acid (1 mL) was added at 0° C. under nitrogen. The reaction solution was stirred at room temperature for 2 hours, concentrated, and then purified by HPLC to afford Intermediate Compound 23 (26 mg, 46%).

¹H-NMR (400 MHz, DMSO-d₆), δ 8.66 (br s, 1H), 7.96 (d, 2H), 7.65 (s, 2H), 7.37 (br s, 2H), 7.30 (d, 2H), 6.53 (d, 2H), 5.86-5.63 (m, 4H), 4.93-4.89 (m, 4H), 4.56-4.50 (m, 6H), 3.71 (s, 3H), 2.11 (d, 6H) 1.30-1.25 (m, 6H). EI-MS m/z: [M+H]⁺ 861.21.

### Example 4: Preparation of Intermediate Compound 28

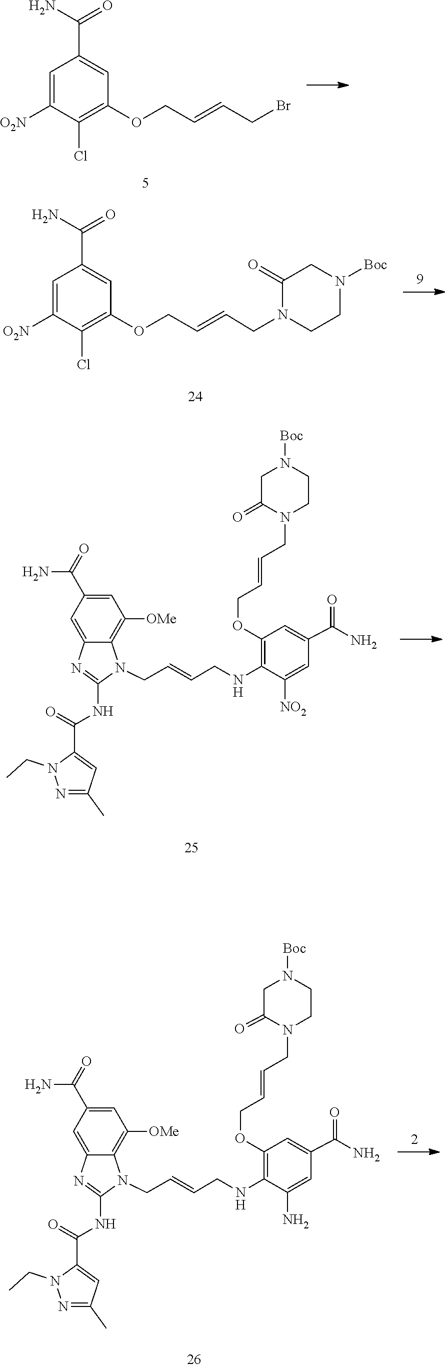 [SMILES: NC(=O)c1cc(OC/C=C/CBr)c(Cl)c([N+](=O)[O-])c1 ; CC(C)(C)OC(=O)N1CCN(C/C=C/COc2cc(C(N)=O)cc([N+](=O)[O-])c2Cl)C(=O)C1 ; CCn1nc(C)cc1C(=O)Nc1nc2cc(C(N)=O)cc(OC)c2n1C/C=C/CNc1c(OC/C=C/CN2CCN(C(=O)OC(C)(C)C)CC2=O)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C/CN1CCN(C(=O)OC(C)(C)C)CC1=O]

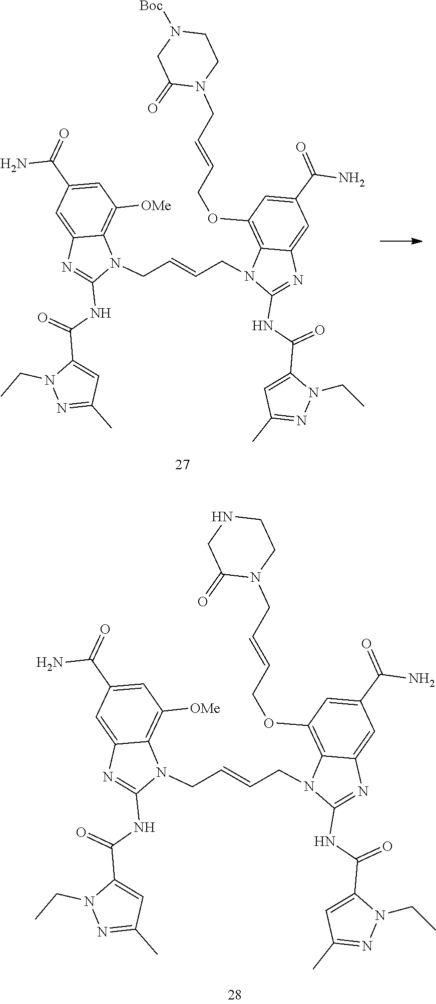

### Preparation of Intermediate Compound 24

t-Butyl 3-oxopiperazine-1-carboxylate (300 mg, 0.86 mmol) was dissolved in tetrahydrofuran (4 mL), and potassium hydroxide (57.7 mg, 1.02 mmol) and TBAB (tetrabutylammonium bromide, 55.3 mg, 0.17 mmol) were sequentially added, and then stirred at room temperature for 30 minutes. Intermediate Compound 5 (300 mg, 0.858 mmol) was dissolved in THF (2 mL), and then slowly added to the reaction solution, and stirred at room temperature for 2 hours. The reaction solution was diluted with ethyl acetate (50 mL), washed with distilled water (30 mL), and then dried with anhydrous magnesium sulfate. The reaction solution was filtered, concentrated under reduced pressure, and purified by column chromatography to afford an Intermediate Compound 24 (213 mg, 52.90%).

¹H-NMR (400 MHz, CDCl₃), δ 8.26 (br s, 1H), 8.06 (s, 1H), 7.87 (s, 1H), 7.78 (s, 1H), 5.90-5.80 (m, 2H), 4.84 (d, J=4.4 Hz, 1H), 4.00 (d, J=4.8 Hz, 2H), 3.92 (s, 2H), 3.53-3.51 (m, 2H), 3.27-3.24 (m, 2H), 1.41 (s, 9H). EI-MS m/z: [M+H]⁺ 469.07.

### Preparation of Intermediate Compound 25

After dissolving Intermediate Compound 9 (440 mg, 0.91 mmol) and Intermediate Compound 24 (213 mg, 0.45 mmol) in normal butyl alcohol (4.5 mL), diisopropylethylamine (0.43 mL, 2.49 mmol) was added at room temperature and stirred for 24 hours while heating to 120° C. The reaction solution was cooled to room temperature and then diluted with dichloromethane (100 mL) and methanol (20 mL) and washed with distilled water (50 mL). The organic layer was dried with anhydrous sodium sulfate, filtered and concentrated under reduced pressure. The solid obtained by diluting with dichloromethane and diethyl ether was filtered and dried to afford Intermediate Compound 25 (213 mg, 55.5%). EI-MS m/z: [M+H]⁺ 844.04.

### Preparation of Intermediate Compound 26

After dissolving Intermediate Compound 25 (210 mg, 0.25 mmol) in methanol (6 mL) and distilled water (1 mL), an aqueous ammonia solution (28-30%, 0.44 mL) and sodium hydrosulfite (Na₂S₂O₄, 433 mg, 2.48 mmol) were added to the reaction solution under nitrogen. The mixture was stirred at room temperature for 1 hour and 45 minutes, diluted with methanol (50 mL), and then filtered. The filtrate was concentrated under reduced pressure, diluted with dichloromethane (100 mL) and methanol (20 mL), and then washed with distilled water (50 mL). The organic layer was dried with anhydrous sodium sulfate and filtered. The filtrate was concentrated under reduced pressure to afford Intermediate Compound 26 (crude). EI-MS m/z: [M+H]⁺ 814.14.

### Preparation of Intermediate Compound 27

After dissolving Intermediate Compound 26 (0.24 mmol, crude) in N,N-dimethylformamide (2.5 mL), Intermediate Compound 2 (50.9 mg, 0.26 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (68.1 mg, 0.35 mmol) and triethylamine (0.1 mL, 0.71 mmol) were added and stirred at room temperature for 18 hours and 30 minutes. The reaction solution was concentrated under reduced pressure and purified by column chromatography to afford Intermediate Compound 27 (48 mg, 20%). EI-MS m/z: [M+H]⁺ 975.19.

### Preparation of Intermediate Compound 28

After dissolving Intermediate Compound 27 (48 mg) in dichloromethane (3.2 mL), trifluoroacetic acid (0.8 mL) was added at 0° C. under nitrogen. The reaction solution was stirred at 0° C. for 40 minutes, concentrated, and then purified by HPLC to afford Intermediate Compound 28 (10.2 mg, 17%).

¹H-NMR (400 MHz, DMSO-d₆), δ 1.28 (br s, 2H), 9.13 (br s, 1H), 7.96 (d, J=14.7 Hz, 2H), 7.65 (s, 2H), 7.37 (br s, 2H), 7.30 (s, 2H), 6.53 (d, J=6.3 Hz, 2H), 5.86-5.63 (m, 4H), 4.92-4.89 (m, 4H), 4.56-4.50 (m, 4H), 3.31 (s, 5H), 2.11 (d, J=4.9 Hz, 6H) 1.29-1.25 (m, 6H). EI-MS m/z: [M+H]⁺ 875.11.

### Example 5: Preparation of Intermediate Compound 33

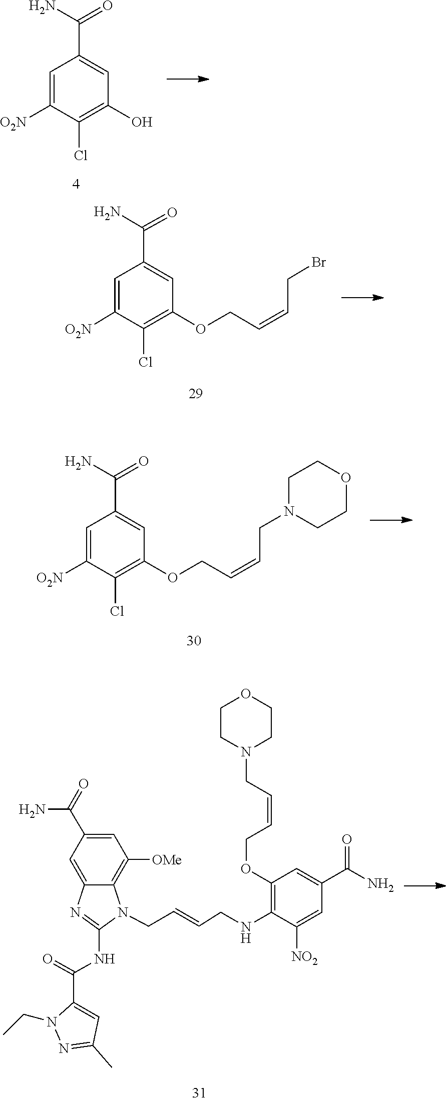

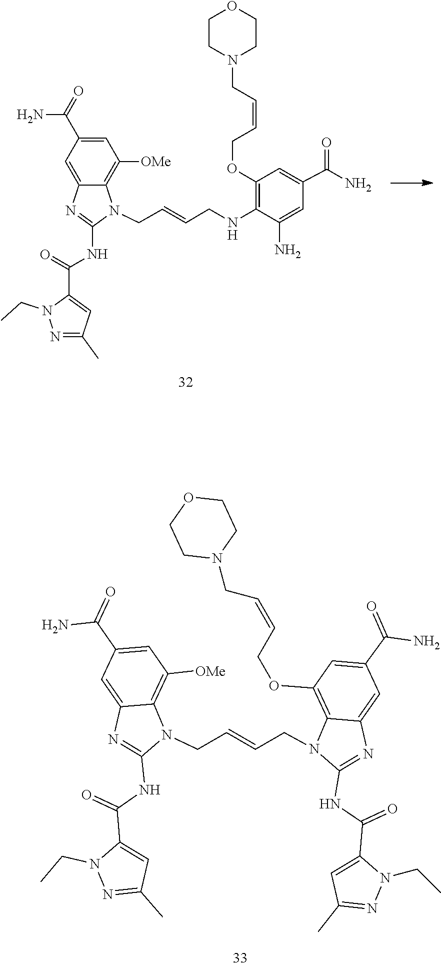 [SMILES: CCn1nc(C)cc1C(=O)Nc1nc2cc(C(N)=O)cc(OC)c2n1C/C=C/CNc1c(N)cc(C(N)=O)cc1OC/C=C\CN1CCOCC1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C\CN3CCOCC3)c21]

### Preparation of Intermediate Compound 29

Intermediate Compound 4 (1.5 g, 6.93 mmol) and cesium carbonate (2.5 g, 7.62 mmol) were dissolved in N,N-dimethylformamide (6 mL) at 0° C. under nitrogen, and then stirred for 5 minutes. Cis-1,4-dibromo-2-butene (3.7 g, 17.32 mmol) was added to the reaction solution at room temperature under nitrogen and stirred for 2 hours. The reaction solution was diluted with ethyl acetate (50 mL) and washed with distilled water (20 mL×2) and brine (20 mL). The organic layer was dried with anhydrous sodium sulfate, filtered and concentrated. The solid obtained by diluting with dichloromethane and hexane was filtered and dried to afford Intermediate Compound 29 (1.64 g, 67%). EI-MS m/z: [M+H]⁺ 351.23.

### Preparation of Intermediate Compound 30

After dissolving Intermediate Compound 29 (450 mg, 1.29 mmol) in N,N-dimethylformamide (5 mL), morpholine (0.1 mL, 1.17 mmol) and cesium carbonate (417 mg, 1.28 mmol) were added under nitrogen. The reaction solution was stirred at room temperature for 2 hours, diluted with ethyl acetate (50 mL), washed with distilled water (50 mL×2), and then dried with anhydrous sodium sulfate. The reaction solution was filtered and concentrated under reduced pressure, and the solid obtained by diluting with dichloromethane and hexane was filtered and dried to afford Intermediate Compound 30 (298 mg, 71%). EI-MS m/z: [M+H]⁺ 356.11.

### Preparation of Intermediate Compound 31

After dissolving Intermediate Compound 9 (592 mg, 1.22 mmol) and Intermediate Compound 30 (290 mg, 0.82 mmol) in normal butyl alcohol (6 mL), diisopropylethylamine (0.71 mL, 4.08 mmol) was added at room temperature and stirred for 24 hours while heating to 120° C. The reaction solution was cooled to room temperature, and then diluted with dichloromethane (100 mL) and methanol (20 mL) and washed with distilled water (50 mL). The reaction solution was dried with anhydrous sodium sulfate, filtered, and then concentrated under reduced pressure. The solid obtained by diluting with dichloromethane and diethyl ether was filtered and dried to afford Intermediate Compound 31 (174 mg, 29%). EI-MS m/z: [M+H]⁺ 731.05.

### Preparation of Intermediate Compound 32

After dissolving Intermediate Compound 31 (173 mg, 0.24 mmol) in methanol (2 mL) and distilled water (0.1 mL), an aqueous ammonia solution (28˜30%, 0.34 mL, 4.76 mmol) and sodium hydrosulfite (Na₂S₂O₄, 412 mg, 2.37 mmol) were added to the reaction solution under nitrogen. The mixture was stirred at room temperature for 1 hour, diluted with methanol (50 mL), and filtered. The filtrate was concentrated under reduced pressure, diluted with dichloromethane (100 mL) and methanol (20 mL), and then washed with distilled water (50 mL). The washed material was dried with anhydrous sodium sulfate, filtered, and then concentrated under reduced pressure to afford Intermediate Compound 32 (48 mg, crude, 29%). EI-MS m/z: [M+H]⁺ 701.22.

### Preparation of Intermediate Compound 33

After dissolving Intermediate Compound 32 (48 mg, 0.07 mmol, crude) in N,N-dimethylformamide (1 mL), Intermediate Compound 2 (16 mg, 0.08 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (16 mg, 0.09 mmol) and triethylamine (0.03 mL, 0.2 mmol) were added, and stirred at room temperature for 16 hours. The reaction solution was concentrated under reduced pressure and purified by HPLC to afford Intermediate Compound 33 (17.5 mg, 30%). EI-MS m/z: [M+H]⁺ 862.08.

### Example 6: Preparation of Intermediate Compound 38

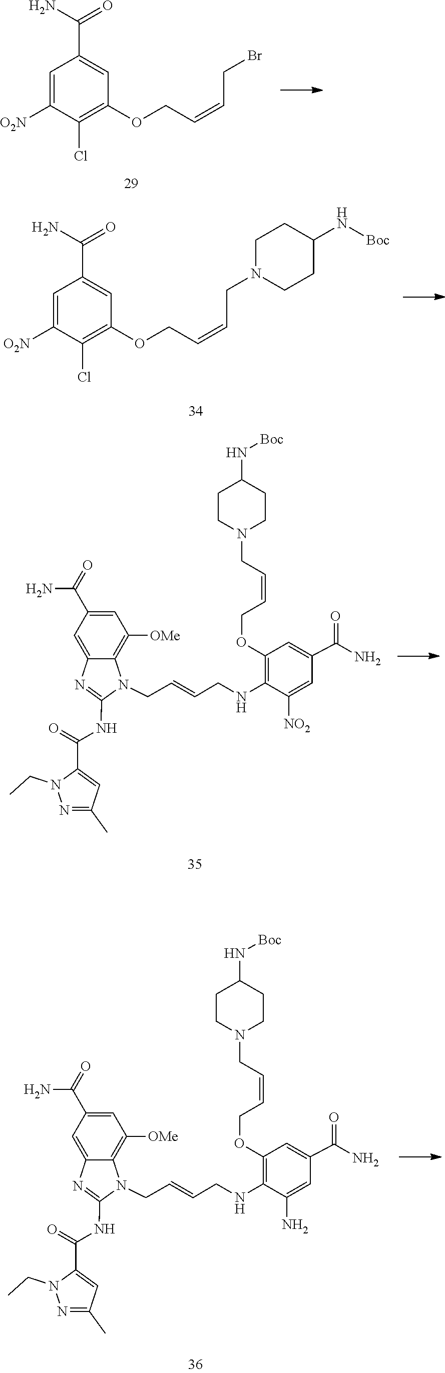 [SMILES: NC(=O)c1cc(OC/C=C\CBr)c(Cl)c([N+](=O)[O-])c1 ; CC(C)(C)OC(=O)NC1CCN(C/C=C\COc2cc(C(N)=O)cc([N+](=O)[O-])c2Cl)CC1 ; CCn1nc(C)cc1C(=O)Nc1nc2cc(C(N)=O)cc(OC)c2n1C/C=C/CNc1c(OC/C=C\CN2CCC(NC(=O)OC(C)(C)C)CC2)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C\CN1CCC(NC(=O)OC(C)(C)C)CC1]

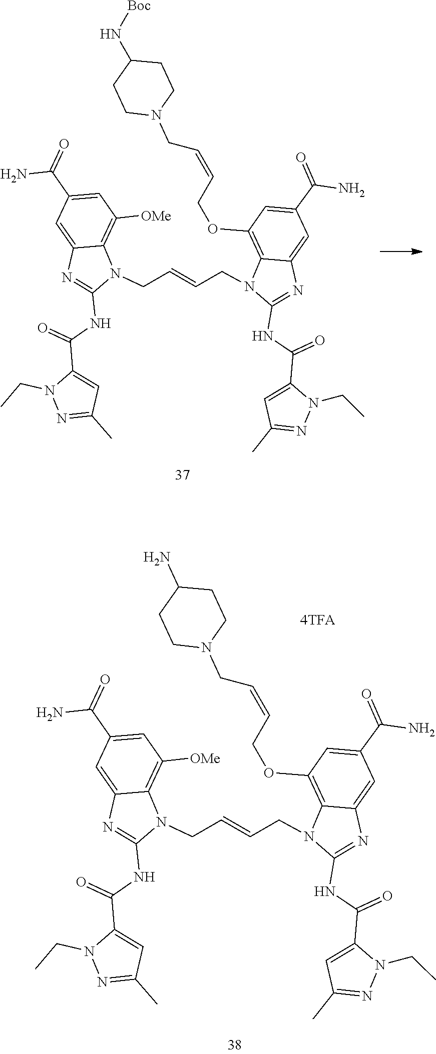

### Preparation of Intermediate Compound 34

After dissolving Intermediate Compound 29 (649 mg, 1.86 mmol) in N,N-dimethylformamide (6 mL), t-butyl piperidin-4-ylcarbamate (338 mg, 1.69 mmol) and cesium carbonate (660 mg, 2.03 mmol) were added under nitrogen. The reaction solution was stirred at room temperature for 2 hours, diluted with ethyl acetate (50 mL), washed with distilled water (50 mL×2), and then dried with anhydrous sodium sulfate. The obtained material was filtered and concentrated under reduced pressure, and the solid obtained by diluting with dichloromethane and hexane was filtered and dried to afford Intermediate Compound 34 (628 mg, 79%).

¹H-NMR (400 MHz, DMSO), δ 8.21 (s, 1H), 8.01 (s, 1H), 7.83 (s, 1H), 7.73 (s, 1H), 6.16-6.01 (m, 2H), 4.82 (d, J=3.5 Hz, 2H), 4.17 (d, J=5.5 Hz, 2H). EI-MS m/z: [M+H]⁺ 469.49.

### Preparation of Intermediate Compound 35

After dissolving Intermediate Compound 9 (413 mg, 0.85 mmol) and Intermediate Compound 34 (600 mg, 1.28 mmol) in normal butyl alcohol (8 mL), diisopropylethylamine (0.74 mL, 4.27 mmol) was added at room temperature and stirred for 24 hours while heating to 120° C. The reaction solution was cooled to room temperature, and then diluted with dichloromethane (100 mL) and methanol (20 mL) and washed with distilled water (50 mL). The washed material was dried with anhydrous sodium sulfate, filtered, and then concentrated under reduced pressure. The solid obtained by diluting with dichloromethane and diethyl ether was filtered and dried to afford Intermediate Compound 35 (147 mg, 20%). EI-MS m/z: [M+H]⁺ 844.13.

### Preparation of Intermediate Compound 36

After dissolving Intermediate Compound 35 (147 mg, 0.17 mmol) in methanol (3 mL) and distilled water (0.1 mL), an aqueous ammonia solution (28˜30%, 0.25 mL) and sodium hydrosulfite (Na₂S₂O₄, 303 mg, 1.74 mmol) were added to the reaction solution under nitrogen. The mixture was stirred at room temperature for 1 hour, diluted with methanol (50 mL), and then filtered. The filtrate was concentrated under reduced pressure, diluted with dichloromethane (100 mL) and methanol (20 mL), and then washed with distilled water (50 mL). The washed material was dried with anhydrous sodium sulfate, filtered, and then concentrated under reduced pressure to afford Intermediate Compound 36 (141 mg, crude). EI-MS m/z: [M+H]⁺ 814.81.

### Preparation of Intermediate Compound 37

After dissolving Intermediate Compound 36 (141 mg, 0.17 mmol, crude) in N,N-dimethylformamide (1 mL), Compound 2 (37 mg, 0.19 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (50 mg, 0.09 mmol) and triethylamine (0.03 mL, 0.22 mmol) were added and stirred at room temperature for 16 hours. The reaction solution was concentrated under reduced pressure and purified by column chromatography to afford Intermediate Compound 37 (48 mg, 28%). EI-MS m/z: [M+H]⁺ 975.16.

### Preparation of Intermediate Compound 38

After dissolving Intermediate Compound 37 (48 mg) in dichloromethane (1 mL), trifluoroacetic acid (1 mL) was added at 0° C. under nitrogen. The reaction solution was stirred at room temperature for 2 hours, concentrated, and then purified by HPLC to afford Intermediate Compound 38 (6.4 mg, 14%). EI-MS m/z: [M+H]⁺ 875.17.

### Example 7: Preparation of Intermediate Compound 43

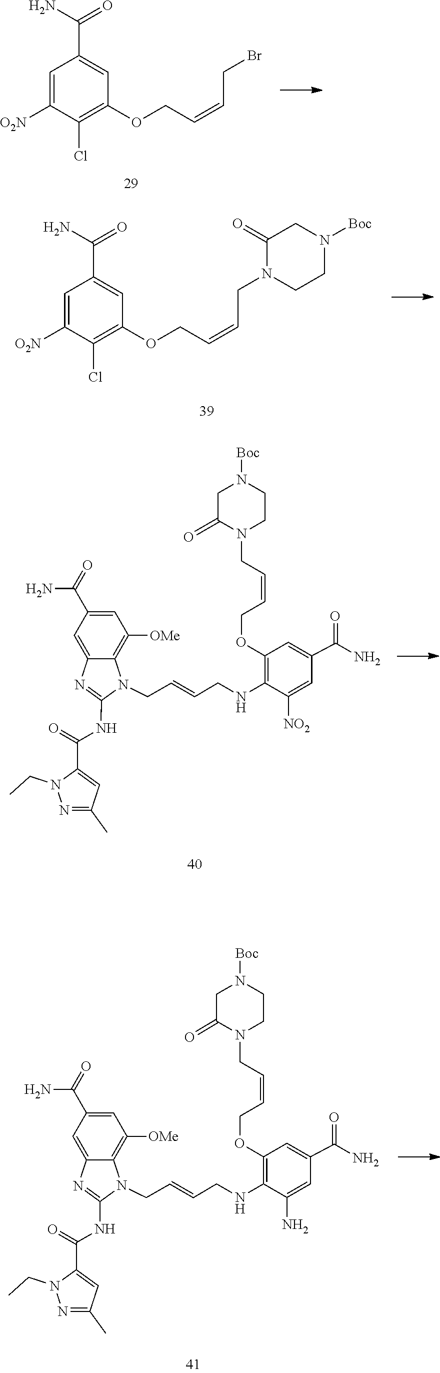 [SMILES: NC(=O)c1cc(OC/C=C\CBr)c(Cl)c([N+](=O)[O-])c1 ; CC(C)(C)OC(=O)N1CCN(C/C=C\COc2cc(C(N)=O)cc([N+](=O)[O-])c2Cl)C(=O)C1 ; CCn1nc(C)cc1C(=O)Nc1nc2cc(C(N)=O)cc(OC)c2n1C/C=C/CNc1c(OC/C=C\CN2CCN(C(=O)OC(C)(C)C)CC2=O)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OC/C=C\CN1CCN(C(=O)OC(C)(C)C)CC1=O]

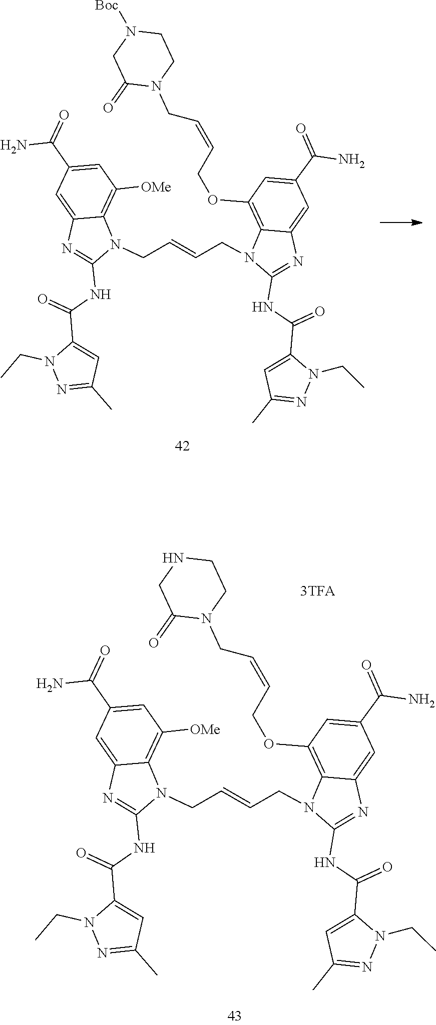

### Preparation of Intermediate Compound 39

t-Butyl 3-oxopiperazine-1-carboxylate (210 mg, 1.05 mmol) was dissolved in N,N-dimethylformamide (6 mL), and potassium hydroxide (66.2 mg, 1.02 mmol) was added and then stirred at room temperature for 30 minutes. After dissolving Intermediate Compound 29 (350 mg, 1.20 mmol) in N,N-dimethylformamide (4 mL), it was slowly added to the reaction solution and stirred at room temperature for 2 hours. The reaction solution was diluted with ethyl acetate (50 mL), washed with distilled water (30 mL), and then dried with anhydrous magnesium sulfate. The dried material was filtered, then concentrated under reduced pressure, and purified by column chromatography to afford Intermediate Compound 39 (432 mg, 92.0%).

¹H-NMR (400 MHz, CDCl₃) δ 7.80 (s, 1H), 7.87 (s, 1H), 7.62 (s, 1H), 5.82-5.81 (m, 2H), 4.76, (d, 1H), 4.10-4.05 (m, 4H), 3.65 (t, 2H), 3.34 (t, 2H), 1.47 (s, 9H). EI-MS m/z: [M+H]⁺ 469.10.

### Preparation of Intermediate Compound 40

After dissolving Intermediate Compound 9 (542 mg, 1.11 mmol) and Intermediate Compound 39 (350 mg, 0.746 mmol) in normal butyl alcohol (4.5 mL), diisopropylethylamine (0.65 mL, 3.73 mmol) was added at room temperature and stirred for 24 hours while heating to 120° C. The reaction solution was cooled to room temperature, and then diluted with dichloromethane (100 mL) and methanol (20 mL) and washed with distilled water (50 mL). The organic layer was dried with anhydrous sodium sulfate, filtered, and then concentrated under reduced pressure. The solid obtained by diluting with dichloromethane and diethyl ether was filtered and dried to afford Intermediate Compound 40 (135 mg, 21.4%). EI-MS m/z: [M+H]⁺ 844.12.

### Preparation of Intermediate Compound 41

After dissolving Intermediate Compound 40 (135 mg, 0.16 mmol) in methanol (6 mL) and distilled water (1 mL), an aqueous ammonia solution (28-30%, 0.3 mL) and sodium hydrosulfite (Na₂S₂O₄, 278 mg, 1.59 mmol) were added to the reaction solution under nitrogen. The mixture was stirred at room temperature for 2 hours, diluted with methanol (50 mL), and then filtered. The filtrate was concentrated under reduced pressure, diluted with dichloromethane (100 mL) and methanol (20 mL), and washed with distilled water (50 mL). The organic layer was dried with anhydrous sodium sulfate, filtered, and then concentrated under reduced pressure to afford Intermediate Compound 41 (91 mg, 69.8%). EI-MS m/z: [M+H]⁺ 814.12.

### Preparation of Intermediate Compound 42

After dissolving Intermediate Compound 41 (91 mg, 0.11 mmol) in N,N-dimethylformamide (2.5 mL), Intermediate Compound 2 (32.7 mg, 0.16 mmol) was dissolved in N,N-dimethylformamide (1 mL) under nitrogen and added thereto. After stirring at room temperature for 1 hour, N-(3-dimethylaminopropyl)-N′-ethylcarbodiimide hydrochloride (34 mg, 0.22 mmol) and triethylamine (0.03 mL, 0.24 mmol) were added and stirred at room temperature for 16 hours. The reaction solution was concentrated under reduced pressure to afford Intermediate Compound 42 (125 mg, crude). EI-MS m/z: [M+H]⁺ 975.17.

### Preparation of Intermediate Compound 43

After dissolving Intermediate Compound 42 (125 mg, crude) in dichloromethane (3.2 mL), trifluoroacetic acid (0.8 mL) was added at 0° C. under nitrogen. The reaction solution was stirred at 0° C. for 40 minutes, concentrated, and then purified by HPLC to afford Intermediate Compound 43 (34 mg, 25%).

¹H-NMR (400 MHz, DMSO-d₆), δ 12.84 (bs, 2H), 9.16 (bs, 2H), 7.97 (d, 2H), 7.65 (s, 2H), 7.36 (s, 2H), 7.30 (s, 2H), 6.53 (d, 2H), 5.79 (s, 2H), 5.73-5.60 (m, 2H), 4.90 (bs, 4H), 4.55-4.50 (m, 6H), 3.84 (d, 2H), 3.73 (s, 2H), 3.69 (s, 4H), 2.11 (d, 6H), 1.29-1.24 (m, 6H). EI-MS m/z: [M+H]⁺ 875.13.

### Example 8: Preparation of Intermediate Compound 48

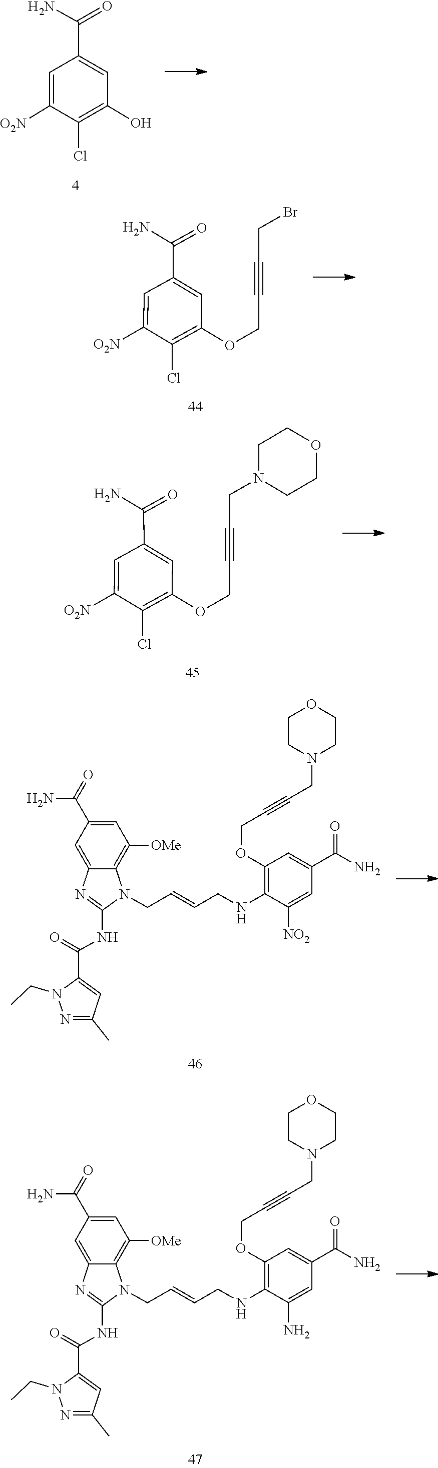 [SMILES: NC(=O)c1cc(O)c(Cl)c([N+](=O)[O-])c1 ; NC(=O)c1cc(OCC#CCBr)c(Cl)c([N+](=O)[O-])c1 ; NC(=O)c1cc(OCC#CCN2CCOCC2)c(Cl)c([N+](=O)[O-])c1 ; CCn1nc(C)cc1C(=O)Nc1nc2cc(C(N)=O)cc(OC)c2n1C/C=C/CNc1c(OCC#CCN2CCOCC2)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OCC#CCN1CCOCC1]

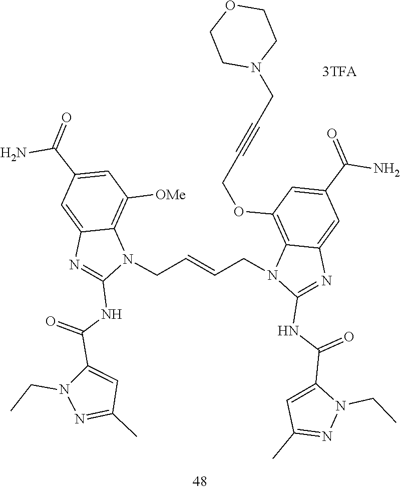 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCC#CCN3CCOCC3)c21]

### Preparation of Intermediate Compound 44

After dissolving Intermediate Compound 4 (2.0 g, 9.23 mmol) in N,N-dimethylformamide (10 mL), 1,4-dibromo-2-butyne (5.8 g, 27.70 mmol) and cesium carbonate (3.6 g, 11.08 mmol) were added under nitrogen. The reaction solution was stirred at room temperature for 2 hours, diluted with ethyl acetate (100 mL), washed with distilled water (50 mL×2), and dried with anhydrous sodium sulfate. The dried material was filtered and concentrated under reduced pressure, and the solid obtained by diluting with dichloromethane and hexane was filtered and dried to afford Intermediate Compound 44 (2.0 g, 62%).

¹H-NMR (400 MHz, DMSO-d₆), δ 8.25 (s, 1H), 8.12 (s, 1H), 7.95 (s, 1H), 7.80 (s, 1H), 5.22 (s, 2H), 4.34 (t, J=5.5 Hz, 2H). EI-MS m/z: [M+H]⁺ 348.99.

### Preparation of Intermediate Compound 45

After dissolving Intermediate Compound 44 (300 mg, 0.86 mmol) in N,N-dimethylformamide (3 mL), morpholine (0.09 mL, 1.03 mmol) and cesium carbonate (309 mg, 0.95 mmol) were added under nitrogen. The mixture was stirred at room temperature for 3 hours, diluted with ethyl acetate (50 mL), washed with distilled water (50 mL×2), and dried with anhydrous sodium sulfate. The dried material was filtered, concentrated under reduced pressure, and purified by column chromatography to afford Intermediate Compound 45 (230 mg, 75%).

¹H-NMR (400 MHz, DMSO-d₆) δ 8.25 (s, 1H), 8.11 (s, 1H), 8.01 (s, 1H), 7.79 (s, 1H), 5.17 (s, 2H), 3.52 (t, J=11.5 Hz, 4H), 3.29 (s, 2H), 2.36 (t, J=11.0 Hz, 4H). EI-MS m/z: [M+H]⁺ 354.13.

### Preparation of Intermediate Compound 46

After dissolving Intermediate Compound 9 (500 mg, 1.03 mmol) and Intermediate Compound 45 (215 mg, 0.60 mmol) in normal butyl alcohol (4 mL), diisopropylethylamine (0.53 mL, 3.03 mmol) was added at room temperature and stirred for 24 hours while heating to 120° C. The reaction solution was cooled to room temperature, and then diluted with dichloromethane (100 mL) and methanol (20 mL) and washed with distilled water (50 mL). The organic layer was dried with anhydrous sodium sulfate and filtered. The filtrate was concentrated under reduced pressure. The solid obtained by diluting with dichloromethane and diethyl ether was filtered and dried to afford Intermediate Compound 46 (303 mg, 68%). EI-MS m/z: [M+H]⁺ 729.11.

### Preparation of Intermediate Compound 47

After dissolving Intermediate Compound 46 (303 mg, 0.41 mmol) in methanol (10 mL) and distilled water (1 mL), an aqueous ammonia solution (28˜30% ammonia, 0.45 mL) and sodium hydrosulfite (Na₂S₂O₄, 724 mg, 4.16 mmol) were added to the reaction solution under nitrogen. The mixture was stirred at room temperature for 1 hour, diluted with methanol (50 mL), and filtered. The filtrate was concentrated under reduced pressure, diluted with dichloromethane (100 mL) and methanol (20 mL), and then washed with distilled water (50 mL). The organic layer was dried with anhydrous sodium sulfate, filtered, and then concentrated under reduced pressure to afford Intermediate Compound 47 (97 mg, 33%, crude). EI-MS m/z: [M+H]⁺ 699.07.

### Preparation of Intermediate Compound 48

After dissolving Intermediate Compound 47 (97 mg, 0.14 mmol, crude) in N,N-dimethylformamide (1.5 mL), Intermediate Compound 2 (32 mg, 0.17 mmol) was dissolved in N,N-dimethylformamide (0.5 mL) under nitrogen and added thereto. After stirring at room temperature for 1 hour, N-(3-dimethylaminopropyl)-N′-ethylcarbodiimide hydrochloride (40 mg, 0.21 mmol) and triethylamine (0.06 mL, 0.42 mmol) were added and stirred at room temperature for 13 hours. The reaction solution was concentrated under pressure and purified by HPLC to afford Intermediate Compound 48 (38 mg, 32%).

¹H-NMR (400 MHz, MeOD-d₄), δ 7.60 (d, J=1.3 Hz, 1H), 7.58 (d, J=1.3 Hz, 1H), 7.44 (d, J=1.4 Hz, 1H), 7.31 (d, J=1.4 Hz, 1H), 6.60 (d, J=0.6 Hz, 1H), 6.58 (d, J=0.6 Hz, 1H), 5.89-5.86 (m, 4H), 5.04-5.01 (m, 4H), 4.61-4.55 (m, 4H), 4.07 (s, 2H), 3.71 (s, 3H), 2.20 (s, 3H), 2.18 (s, 3H), 1.37-1.30 (m, 6H). EI-MS m/z: [M+H]⁺ 860.08.

### Example 9: Preparation of Intermediate Compound 55

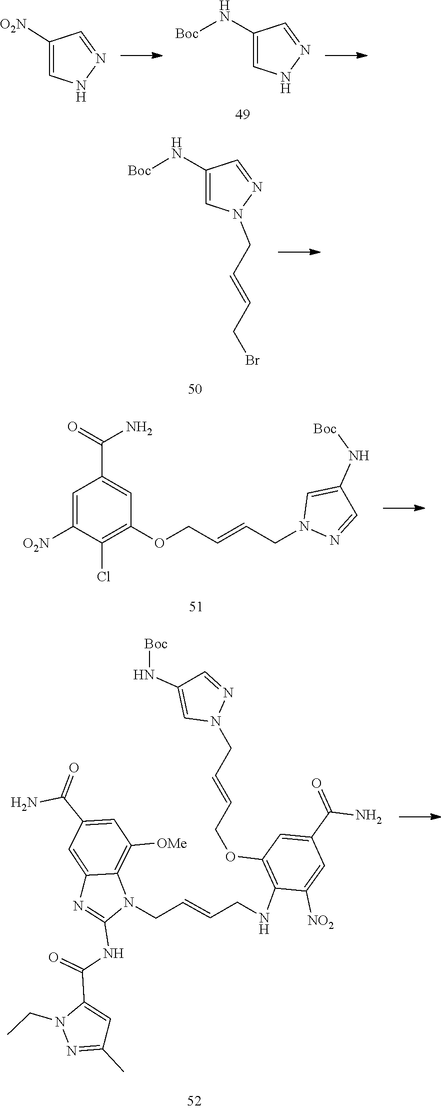 [SMILES: O=[N+]([O-])c1cn[nH]c1 ; CC(C)(C)OC(=O)Nc1cn[nH]c1 ; CC(C)(C)OC(=O)Nc1cnn(C/C=C/CBr)c1 ; CC(C)(C)OC(=O)Nc1cnn(C/C=C/COc2cc(C(N)=O)cc([N+](=O)[O-])c2Cl)c1 ; CCn1nc(C)cc1C(=O)Nc1nc2cc(C(N)=O)cc(OC)c2n1C/C=C/CNc1c(OC/C=C/Cn2cc(NC(=O)OC(C)(C)C)cn2)cc(C(N)=O)cc1[N+](=O)[O-]]

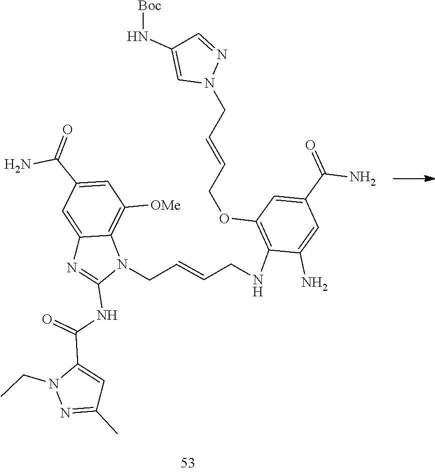 [SMILES: CCn1nc(C)cc1C(=O)Nc1nc2cc(C(N)=O)cc(OC)c2n1C/C=C/CNc1c(N)cc(C(N)=O)cc1OC/C=C/Cn1cc(NC(=O)OC(C)(C)C)cn1]

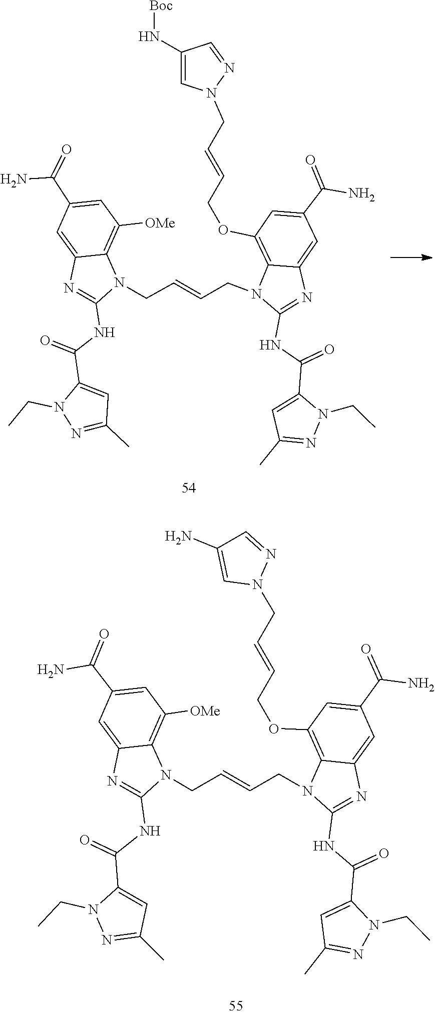

### Preparation of Intermediate Compound 49

To a solution of 4-nitropyrazole (1 g, 6.13 mmol) in methanol (20 mL) were added ammonia solution (28-30% ammonia, 2.2 mL) and sodium hydrosulfite (7.7 g). After stirred at room temperature for 1 hour, the reaction solution was filtered through Celite and the filtrate was evaporated under reduced pressure. Methanol (15 mL) was added to the concentrated filtrate and then di-t-butyl dicarbonate (2.24 mL, 9.73 mmol) and triethylamine (1.86 mL, 13.27 mmol) were added at room temperature. The reaction solution was stirred at room temperature for 16 hours and then diluted with ethyl acetate (50 mL) and washed with distilled water (50 mL×2). The organic layer was dried over anhydrous sodium sulfate and filtered. The filtrate was concentrated under reduced pressure. The reduced was purified by column chromatography to afford Intermediate Compound 49 (0.7 g, 43%).

¹H-NMR (400 MHz, DMSO-d₆) δ 9.05 (s, 1H), 7.44 (s, 1H), 1.42 (s, 9H).

### Preparation of Intermediate Compound 50

To a solution of Intermediate Compound 49 (0.7 g, 3.82 mmol) in N,N-dimethylformamide (15 mL) were added cesium carbonate (3.7 g, 11.46 mmol) and trans-1,4-dibromo-2-butene (2.45 g, 11.46 mmol). After stirred at room temperature for 2 hours, the reaction solution was diluted with ethyl acetate (50 mL) and then washed with saturated aqueous ammonium chloride solution (50×2 mL). The organic layer was dried over anhydrous sodium sulfate and filtered. The filtrate was concentrated under reduced pressure. The residue was purified by column chromatography to afford Intermediate Compound 50 (867 mg, 71%).

¹H-NMR (400 MHz, CDCl₃) δ 7.66 (s, 1H), 7.33 (s, 1H), 6.24 (s, 1H), 6.00-5.90 (m, 1H), 5.87 (td, J=13.2, 5.9 Hz, 1H), 4.69 (d, J=5.6 Hz, 2H), 3.94 (d, J=6.9 Hz, 2H), 1.50 (s, 9H). EI-MS m/z: [M+H]⁺ 317.26.

### Preparation of Intermediate Compound 51

To a solution of Intermediate Compound 4 (540 mg, 2.5 mmol) in N,N-dimethylformamide (15 mL) were added cesium carbonate (894 mg, 2.75 mmol) and compound 50 (867 mg, 2.75 mmol). After stirred at room temperature for 2 hours, the reaction solution was diluted with ethyl acetate (50 mL) and washed with saturated aqueous ammonium chloride solution (50×2 mL). The organic layer was dried over anhydrous sodium sulfate and filtered. The filtrate was concentrated under reduced pressure. The residue was purified by column chromatography to afford Intermediate Compound 51 (830 mg, 73%).

¹H-NMR (400 MHz, DMSO-d₆) δ 9.12 (s, 1H), 8.24 (s, 1H), 8.05 (s, 1H), 7.88 (s, 1H), 7.75 (s, 1H), 7.65 (s, 1H), 7.30 (s, 1H), 6.08 (d, J=15.4 Hz, 1H), 5.86 (d, J=15.8 Hz, 1H), 4.82 (d, J=5.4 Hz, 2H), 4.74 (d, J=5.9 Hz, 2H), 1.44 (d, J=2.3 Hz, 9H). EI-MS m/z: [M+H]⁺ 452.31.

### Preparation of Intermediate Compound 52

To a solution of Intermediate Compound 51 (830 mg, 1.83 mmol) and compound 9 (1.13 g, 2.76 mmol) in n-butanol (11 mL) were added N,N-diisopropylethylamine (1.6 mL, 9.18 mmol). The reaction solution was stirred at 0° C. for 5 minutes then heated to 120° C. for 24 hours. After cooled to room temperature, the reaction solution was concentrated under reduced pressure. The resulting residue was purified by column chromatography to afford Intermediate Compound 52 (420 mg, 28%). EI-MS m/z: [M+H]⁺ 827.39.

### Preparation of Intermediate Compound 53

To a solution of Intermediate Compound 52 (420 mg, 0.51 mmol) in methanol (5 mL) were added aqueous ammonia solution (28˜30% ammonia, 1.8 mL, 12.66 mmol) and sodium hydrosulfite (882 mg, 5.06 mmol). After stirred at room temperature for 2.5 hours, the reaction solution was filtered through Celite and washed with methanol. The filtrate was concentrated under reduced pressure and purified by reversed phase column chromatography to afford Intermediate Compound 53 (400 mg). EI-MS m/z: [M+H]⁺ 797.48.

### Preparation of Intermediate Compound 54

To a solution of Intermediate Compound 53 (400 mg, 0.51 mmol) in N,N-dimethylformamide (1.5 mL) at 0° C. was added compound 2 (109 mg, 0.55 mmol) in N,N-dimethylformamide (1 mL). After 30 minutes, N-(3-dimethylaminopropyl)-N-ethyl carbodiimide (0.1 mL, 0.61 mmol) and triethylamine (0.35 mL, 2.53 mmol) were added to the reaction solution. After stirred at room temperature for 15 hours, the reaction solution was concentrated under reduced pressure and purified by reverse phase chromatography to afford Intermediate Compound 54 (97 mg, 20%).

### Preparation of Intermediate Compound 55

To a solution of Intermediate Compound 54 (30 mg, 0.03 mmol) in dichloromethane (1 mL) was added trifluoroacetic acid (0.2 mL) at 0° C. After stirred at room temperature for 30 minutes, the reaction mixture was concentrated and purified by HPLC to afford Intermediate Compound 55 (10 mg, 27%).

¹H-NMR (400 MHz, CD₃OD) δ 7.80 (s, 1H), 7.57 (d, J=11.4 Hz, 3H), 7.29 (s, 1H), 7.23 (s, 1H), 6.62 (d, J=1.7 Hz, 1H), 6.56 (s, 1H), 5.85 (d, J=18.0 Hz, 3H), 5.70 (d, J=15.6 Hz, 1H), 5.01 (s, 4H), 4.63 (s, 3H), 4.61-4.52 (m, 2H), 4.45 (s, 2H), 3.74 (s, 2H), 3.31 (m, 3H), 2.65 (s, 1H), 2.20 (d, J=12.3 Hz, 6H), 1.34 (dt, J=21.7, 6.9 Hz, 6H). EI-MS m/z: [M+H]⁺ 858.54.

### Example 10: Preparation of Intermediate Compound 65

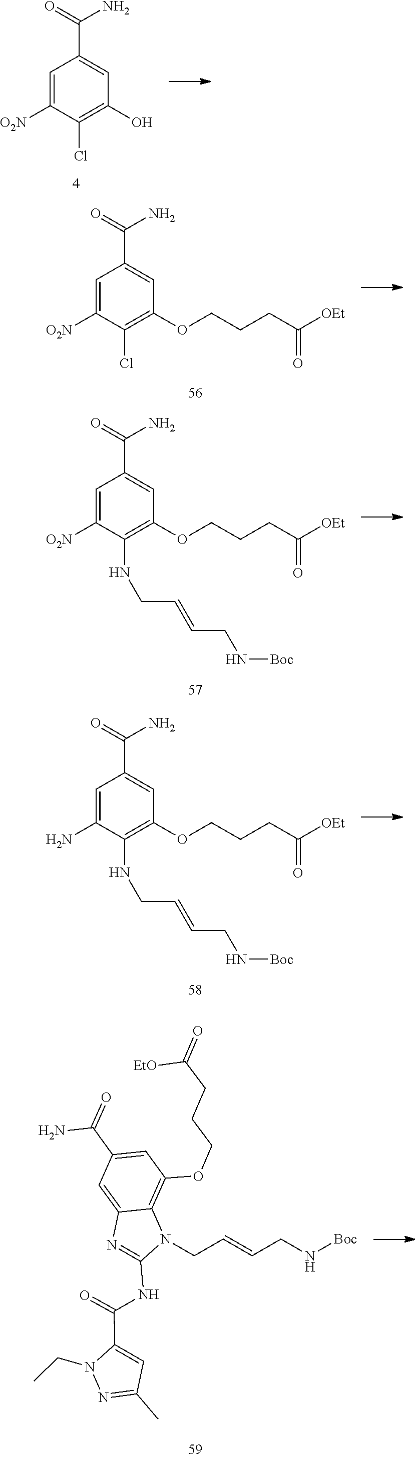 [SMILES: NC(=O)c1cc(O)c(Cl)c([N+](=O)[O-])c1 ; CCOC(=O)CCCOc1cc(C(N)=O)cc([N+](=O)[O-])c1Cl ; CCOC(=O)CCCOc1cc(C(N)=O)cc([N+](=O)[O-])c1NC/C=C/CNC(=O)OC(C)(C)C ; CCOC(=O)CCCOc1cc(C(N)=O)cc(N)c1NC/C=C/CNC(=O)OC(C)(C)C ; CCOC(=O)CCCOc1cc(C(N)=O)cc2nc(NC(=O)c3cc(C)nn3CC)n(C/C=C/CNC(=O)OC(C)(C)C)c12]

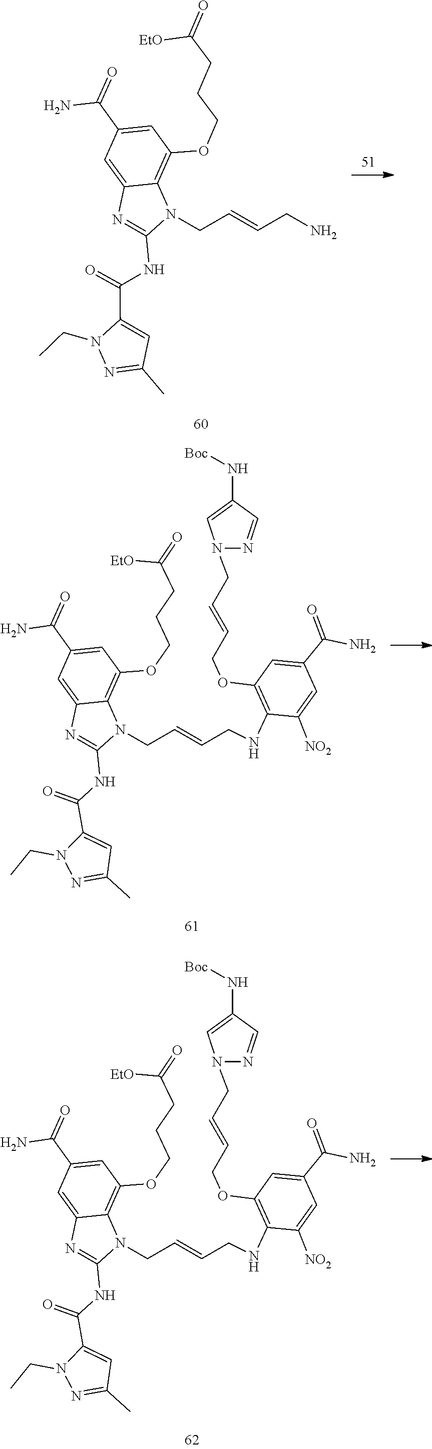 [SMILES: CCOC(=O)CCCOc1cc(C(N)=O)cc2nc(NC(=O)c3cc(C)nn3CC)n(C/C=C/CN)c12 ; CCOC(=O)CCCOc1cc(C(N)=O)cc2nc(NC(=O)c3cc(C)nn3CC)n(C/C=C/CNc3c(OC/C=C/Cn4cc(NC(=O)OC(C)(C)C)cn4)cc(C(N)=O)cc3[N+](=O)[O-])c12 ; CCOC(=O)CCCOc1cc(C(N)=O)cc2nc(NC(=O)c3cc(C)nn3CC)n(C/C=C/CNc3c(OC/C=C/Cn4cc(NC(=O)OC(C)(C)C)cn4)cc(C(N)=O)cc3[N+](=O)[O-])c12]

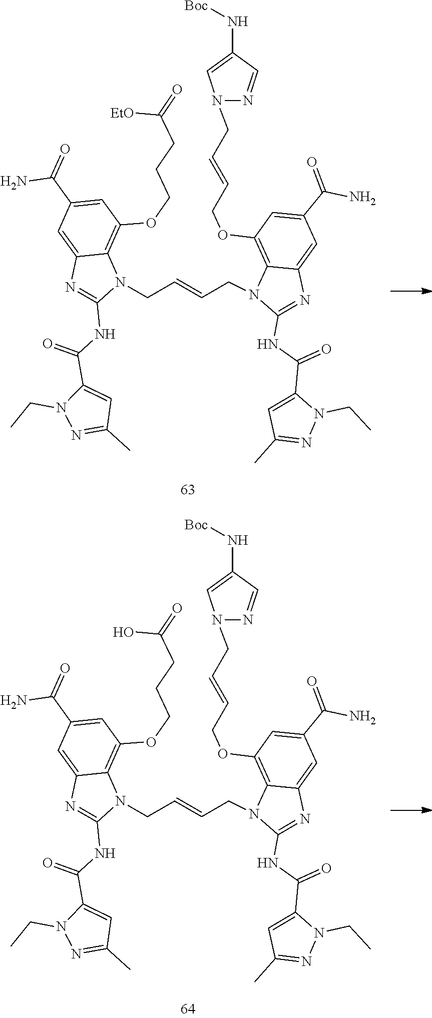

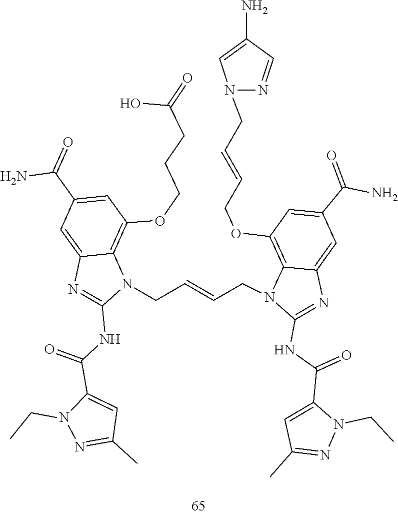 [SMILES: CCn1nc(C)cc1C(=O)Nc1nc2cc(C(N)=O)cc(OC/C=C/Cn3cc(N)cn3)c2n1C/C=C/Cn1c(NC(=O)c2cc(C)nn2CC)nc2cc(C(N)=O)cc(OCCCC(=O)O)c21]

### Preparation of Intermediate Compound 56

To a solution of Intermediate Compound 4 (5 g, 23.08 mmol) in N,N-dimethylformamide (30 mL) was added cesium carbonate (11.2 g, 34.62 mmol) at 0° C. under nitrogen. After 5 minutes, ethyl 4-bromobutyrate (5.4 g, 27.70 mmol) was added to the reaction solution at room temperature under nitrogen. After stirred for 2 hours, the reaction solution was diluted with ethyl acetate (60 mL) and washed with distilled water (15 mL×2) and brine (15 mL). The organic layer was dried over anhydrous sodium sulfate and filtered. The filtrate was concentrated. After solidification used dichloromethane and hexane, the resulting solid was filtered and dried to afford Intermediate Compound 56 (4.7 g, 61%), which was used without further purification.

¹H-NMR (400 MHz, CDCl₃) δ 8.28 (s, 1H), 8.04 (s, 1H), 7.86 (s, 1H), 7.73 (s, 1H), 4.25 (m, 2H), 4.09-4.04 (q, J=7.2 Hz, 2H), 1.19-1.15 (t, J=7.2 Hz, 3H). EI-MS m/z: [M+H]⁺ 331.20.

### Preparation of Intermediate Compound 57

To a solution of Intermediate Compound 56 (4.5 g, 13.60 mmol) in ethanol (30 mL) were added t-butyl (E)-(4-aminobut-2-en-1-yl)carbamate (2.5 g, 13.60 mmol) and N,N-diisopropylethylamine (2.37 mL, 27.21 mmol). After stirred at 120° C. for 12 hours, the reaction solution was cooled to room temperature. The reaction mixture was diluted ethyl acetate (60 mL) and washed with distilled water (15 mL×2) and brine (15 mL). The organic layer was dried over anhydrous sodium sulfate filtered and concentrated. The resulting residue was purified by column chromatography to afford Intermediate Compound 57 (4.5 g, 68%).

¹H-NMR (400 MHz, DMSO-d₆) δ 8.10 (s, 1H), 8.01 (s, 1H), 7.68 (t, 1H), 7.58 (s, 1H), 7.30 (s, 1H), 6.90 (s, 1H), 5.54 (s, 2H), 4.10 (m, 6H), 3.48 (s, 3H), 2.07 (m, 2H) 1.35 (m, 9H) 1.17 (m, 4H). EI-MS m/z: [M+H]⁺ 481.28.

### Preparation of Intermediate Compound 58

To a solution of Intermediate Compound 57 (4.4 g, 9.156 mmol) in methanol (20 mL) were added aqueous ammonia solution (28˜30% ammonia, 10 mL) and sodium hydrosulfite (Na₂S₂O₄, 15 g, 91.6 mmol) at 0° C. After stirred at room temperature for 1.5 hours, the reaction solution was filtered through Celite with methanol. The filtrate was concentrated under reduced pressure to afford Intermediate Compound 58 (4 g, 96%). EI-MS m/z: [M+H]⁺ 451.31.

### Preparation of Intermediate Compound 59

To a solution of Intermediate Compound 58 (4.0 g, 8.87 mmol) in N,N-dimethylformamide (30 mL) was added compound 2 (1.9 g, 9.76 mmol) at 0° C. After stirred at room temperature for 30 min, triethylamine (3.7 mL, 26.63 mmol) and N-(3-dimethylaminopropyl)-N-ethyl carbodiimide (2.7 g, 17.75 mmol) were added to the reaction solution at 0° C. The resulting reaction solution was stirred at room temperature for 17 hours. Then, the reaction solution was concentrated, and the resulting residue was purified by column chromatography to afford Intermediate Compound 59 (3.8 g, 71%).

¹H-NMR (400 MHz, DMSO-d₆) δ 12.86 (s, 1H), 7.99 (s, 1H), 7.66 (s, 1H), 7.37-7.35 (m, 2H), 6.89 (m, 1H), 6.63 (s, 1H), 5.79-5.72 (d, J=16 Hz, 1H), 5.58-5.54 (d, J=16 Hz, 1H) 4.94 (s, 2H), 4.62 (m, 2H), 4.22 (s, 3H), 4.20 (m, 6H), 2.31 (m, 3H), 2.11 (s, 2H), 1.36 (m, 9H), 1.17 (m, 4H). EI-MS m/z: [M+H]⁺ 612.31.

### Preparation of Intermediate Compound 60

To a solution of Intermediate Compound 59 (3.8 g, 6.21 mmol) in dichloromethane (50 mL) was added hydrochloric acid (4 M 1,4-dioxane solution, 11.5 mL). After stirred for 2 hours, the reaction solution was concentrated, and diluted with diethyl ether (20 mL). The resulting solid was filtered to afford Intermediate Compound 60 (3.9 g, 99%).

¹H-NMR (400 MHz, DMSO-d₆) δ 8.04 (s, 1H), 7.88 (m, 2H), 7.68 (s, 1H), 7.39 (s, 2H), 6.67 (s, 1H), 6.08-6.04 (d, J=16 Hz, 1H), 5.59-5.55 (d, J=16 Hz, 1H), 5.00 (s, 2H), 4.61 (m, 2H), 4.22 (m, 2H), 4.10 (m, 2H), 2.19 (s, 3H), 2.11 (m, 2H), 1.37 (m, 3H), 1.19 (m, 3H). EI-MS m/z: [M+H]⁺ 512.31.

### Preparation of Intermediate Compound 61

To a solution of Intermediate Compound 60 (3.8 g, 6.63 mmol) and Intermediate Compound 51 (2 g, 4.42 mmol) in n-butanol (13 mL) was added N,N-diisopropylethylamine (3.85 mL, 22.13 mmol) at room temperature. After stirred at 100° C. for 21 hours, the reaction solution was cooled to room temperature. The reaction solution was diluted with dichloromethane (100 mL) and methanol (20 mL) and washed with distilled water (50 mL). The combined organic layers were dried over anhydrous sodium sulfate and filtered. The filtrate was concentrated under reduced pressure and purified by column chromatography to afford Intermediate Compound 61 (1.6 g, 38%). EI-MS m/z: [M+H]⁺ 926.98.

### Preparation of Intermediate Compound 62

To a solution of Intermediate Compound 61 (1.6 g, 1.83 mmol) in methanol (8 mL) were added ammonia solution (28-30% ammonia, 3.2 mL) and sodium hydrosulfite (Na₂S₂O₄, 3.1 g, 18.3 mmol) under nitrogen. The reaction solution was stirred at room temperature for 1 hour and then methanol (50 mL) was added, and the resulting solid was filtered out. The filtrate was concentrated and diluted with dichloromethane (100 mL) and washed with distilled water (50 mL). The organic layer was dried over anhydrous sodium sulfate and concentrated under reduced pressure to afford Intermediate Compound 62 (1.4 g, 85%). EI-MS m/z: [M+H]⁺ 897.05.

### Preparation of Intermediate Compound 63

To a solution of Intermediate Compound 62 (1.35 g, 1.51 mmol) in N,N-dimethylformamide (10 mL) was added compound 2 (324 mg, 1.66 mmol) in N,N-dimethylformamide (1 mL) under nitrogen. The reaction solution was stirred at room temperature for 1 hour and then N-(3-dimethylaminopropyl)-N′-ethylcarbodiimide hydrochloride (465 mg, 3.02 mmol) and triethylamine (0.63 mL, 4.53 mmol) were added. After stirred at room temperature for 16 hours, the reaction solution was concentrated under reduced pressure and purified by column chromatography to afford Intermediate Compound 63 (560 mg, 35%). EI-MS m/z: [M+H]⁺ 1057.96.

### Preparation of Intermediate Compound 64

To a solution of Intermediate Compound 63 (100 mg, 0.094 mmol) in methanol (2 mL) was added lithium hydroxide monohydrate (13.8 mg, 0.28 mmol) in distilled water (1 mL) at −50° C. under nitrogen. After stirred at 0° C. for 2 hours, the reaction solution was acidified with acetic acid to pH 4-5 and then concentrated and lyophilized to afford Intermediate Compound 64 (100 mg, crude), which was used without further purification. EI-MS m/z: [M+H]⁺ 1029.95.

### Preparation of Intermediate Compound 65

To a solution of Intermediate Compound 64 (100 mg, 0.097 mmol, crude) in dichloromethane (5 mL) was added trifluoroacetic acid (1 mL) at 0° C. under nitrogen. After stirred at room temperature for 2 hours, the reaction solution was concentrated and purified by HPLC to afford Intermediate Compound 65 (42 mg, 34%).

¹H-NMR (400 MHz, DMSO-d₆) δ 12.80 (s, 1H), 9.77 (s, 2H), 7.96 (s, 1H), 7.87 (m, 2H), 7.65 (s, 2H), 7.53 (s, 1H), 7.34 (s, 2H), 7.28 (s, 2H), 6.53 (s, 2H), 5.79 (m, 3H), 5.65 (m, 1H), 4.90 (s, 4H), 4.62 (m, 2H), 4.50 (m, 6H), 3.92 (m, 3H), 2.24 (s, 2H), 2.11 (m, 6H), 1.74 (m, 2H), 1.26 (m, 6H). EI-MS m/z: [M+H]⁺ 929.98.

### Example 11: Preparation of Intermediate Compound 69

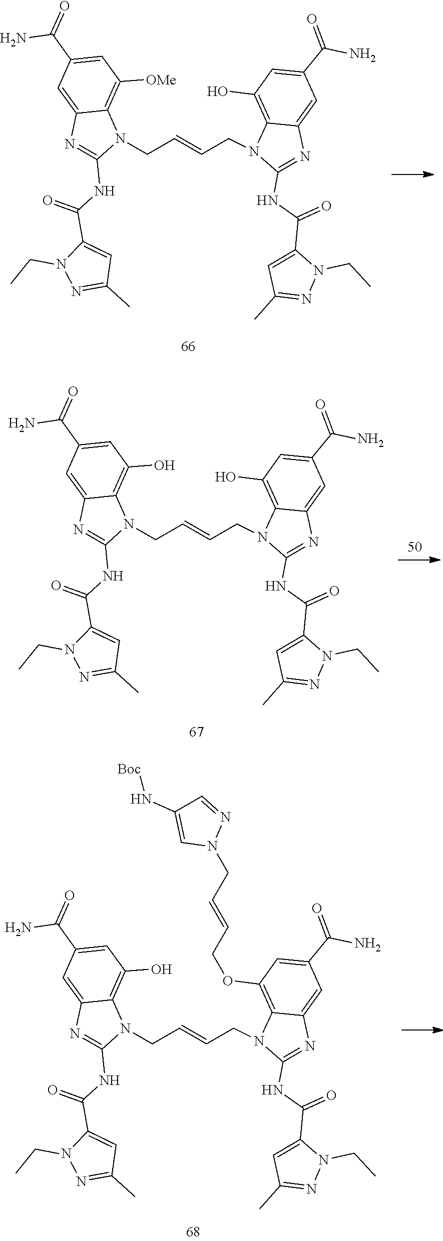

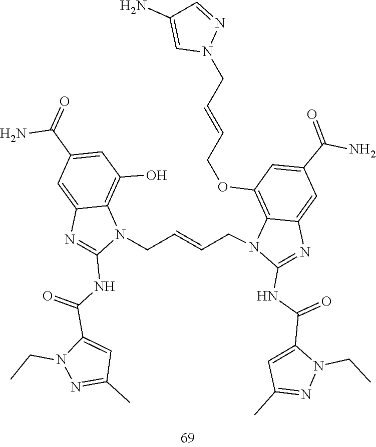 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC/C=C/Cn3cc(N)cn3)c21]

### Preparation of Intermediate Compound 67

To a solution of Intermediate Compound 66 (300 mg, 0.32 mmol, Intermediate Compound 66 was prepared by the method described in the International patent publication No. WO 2022/155518 A1) in dichloroethane (30 mL) was added boron tribromide (1.0 M in dichloromethane, 3.2 mL, 3.16 mmol). After stirred under reflux for 17 hours, the reaction solution was concentrated under reduced pressure and diluted with dichloromethane/diethyl ether (50 mL/50 mL). The resulting solid was filtered to afford Intermediate Compound 67 (280 mg, 84%). EI-MS m/z: [M+H]⁺ 709.15.

### Preparation of Intermediate Compound 68

To a solution of Intermediate Compound 67 (280 mg, 0.27 mmol) in N,N-dimethylformamide (2 mL) were added cesium carbonate (607 mg, 1.87 mmol) and Intermediate Compound 50 (101 mg, 0.32 mmol). After stirred at room temperature for 18 hours, the reaction solution was concentrated under reduced pressure and purified by reversed phase column chromatography to afford Intermediate Compound 68 (158 mg, 62%). EI-MS m/z: [M+H]⁺ 945.00.

### Preparation of Intermediate Compound 69

To a solution of Intermediate Compound 68 (50 mg) in dichloromethane (1.5 mL) was added trifluoroacetic acid (0.5 mL) under nitrogen at 0° C. After stirred at room temperature for 1 hour, the reaction solution was concentrated and the resulting residue was purified by HPLC to afford Intermediate Compound 69 (8 mg).

¹H-NMR (400 MHz, CD₃OD) δ 7.82 (d, J=0.8 Hz, 1H), 7.58 (d, J=0.8 Hz, 1H), 7.53 (d, J=1.3 Hz, 1H), 7.42 (d, J=1.5 Hz, 1H), 7.24 (d, J=1.4 Hz, 1H), 7.17 (d, J=1.4 Hz, 1H), 6.61 (d, J=0.7 Hz, 1H), 6.49 (d, J=0.7 Hz, 1H), 5.95-5.80 (m, 3H), 5.78-5.67 (m, 1H), 5.04 (dd, J=17.4, 3.9 Hz, 4H), 4.67-4.57 (m, 4H), 4.57-4.46 (m, 4H), 2.20 (s, 3H), 2.16 (s, 3H), 1.36 (t, J=7.1 Hz, 3H), 1.28 (t, J=7.1 Hz, 3H). EI-MS m/z: [M+H]⁺ 845.07.

### Example 12: Preparation of Intermediate Compound 79

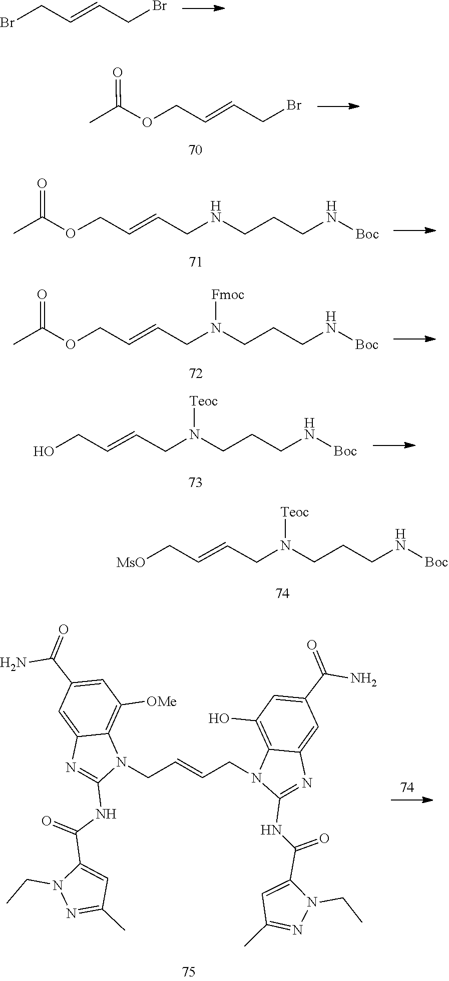

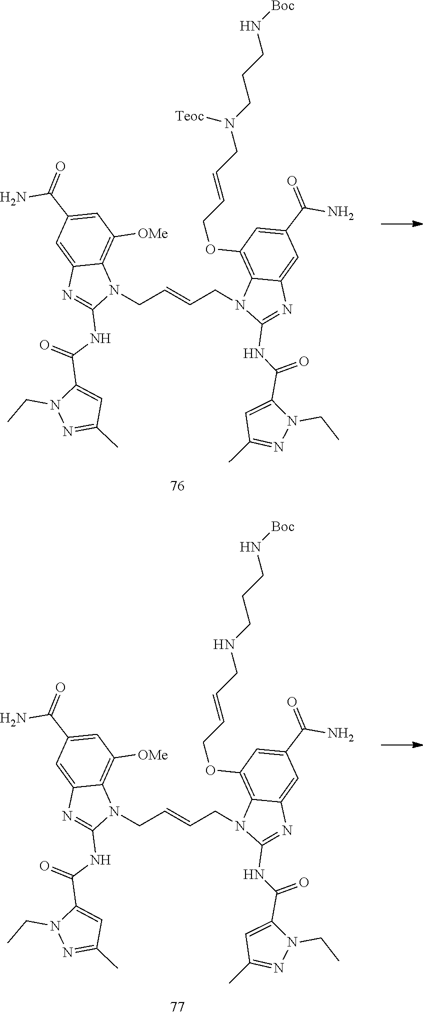

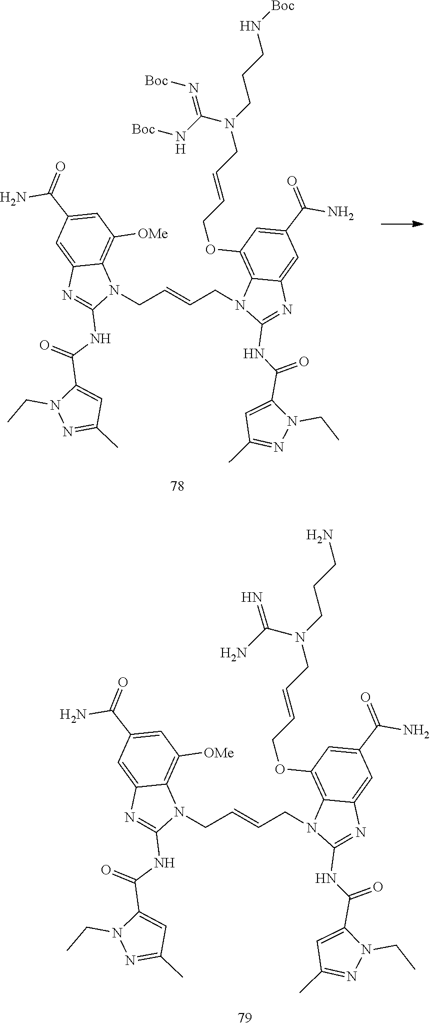

### Preparation of Intermediate Compound 70

To a solution of trans-1,4-dibromo-2-butene (10.4 g, 48.7 mmol) in N,N-dimethylformamide (30 mL) was added sodium acetate (2.0 g, 24.4 mmol) at 0° C. under nitrogen. The reaction solution was stirred at room temperature for 17 hours and then diluted with ethyl acetate (100 mL) and washed with distilled water (50 mL×2). The organic layer was dried over anhydrous sodium sulfate. After filtration and concentration under reduced pressure, the resulting residue was purified by column chromatography to afford Intermediate Compound 70 (2.98 g, 63%).

¹H-NMR (400 MHz, CDCl₃) δ 6.02-5.82 (m, 2H), 4.59 (d, J=5.6 Hz, 2H), 3.95 (d, J=7.2 Hz, 2H), 2.08 (s, 3H).

### Preparation of Intermediate Compound 71

To a solution of Intermediate Compound 70 (838 mg, 4.34 mmol) in dichloromethane (60 mL) were added triethylamine (1.83 mL, 13.02 mmol) and t-butyl(3-aminopropyl) carbamate (2.27 g, 13.02 mmol) in dichloromethane (40 mL) at 0° C. The reaction solution was stirred at room temperature for 18 hours and then concentrated under reduced pressure. The resulting residue was purified by column chromatography to afford Intermediate Compound 71 (380 mg, 30%).

¹H-NMR (400 MHz, CDCl₃) δ 5.87-5.71 (m, 2H), 4.99 (s, 1H), 4.58-4.52 (m, 2H), 3.26 (d, J=5.7 Hz, 2H), 3.23-3.18 (m, 2H), 2.70-2.65 (m, 2H), 2.07 (d, J=2.7 Hz, 3H), 1.68 (d, J=6.6 Hz, 2H), 1.45 (d, J=2.6 Hz, 9H).

### Preparation of Intermediate Compound 72

To a solution of Intermediate Compound 71 (790 mg, 2.76 mmol) in dichloromethane (10 mL) were added fluorenylmethyloxycarbonyl chloride (Fmoc-Cl, 856 mg, 3.31 mmol) and N,N-diisopropylethylamine (0.78 mL, 5.52 mmol). After stirred at room temperature for 2 hours, the reaction solution was diluted with dichloromethane (100 mL) and washed with distilled water (50 mL). The organic layer was dried over anhydrous sodium sulfate. After filtration and concentration under reduced pressure, the resulting residue was purified by column chromatography to afford Intermediate Compound 72 (1.4 g, 98%). EI-MS m/z: [M+H]⁺ 509.19.

### Preparation of Intermediate Compound 73

To a solution of Intermediate Compound 72 (790 mg, 2.76 mmol) in methanol (20 mL) was added potassium carbonate (1.96 g, 14.15 mmol) at 0° C. After stirred at room temperature for 30 minutes, the reaction solution was diluted with dichloromethane (100 mL) and washed with distilled water (50 mL). The organic layer was dried over anhydrous sodium sulfate. After filtration, and concentration under reduced pressure, the resulting residue was dissolved in dichloromethane (10 mL) and then 4-nitrophenyl(2-(trimethylsilyl)ethyl)carbonate (Teoc-PNP, 962 mg, 3.39 mmol) and N,N-diisopropylethylamine (0.80 mL, 5.66 mmol) were added and the reaction solution was stirred at room temperature for 18 hours. The reaction solution was diluted with dichloromethane (100 mL) and washed with distilled water (50 mL). The organic layer was dried over anhydrous sodium sulfate. After filtration and concentration under reduced pressure, the resulting residue was purified by column chromatography to afford Intermediate Compound 73 (596 mg, 54%). EI-MS m/z: [M+H]⁺ 389.28.

### Preparation of Intermediate Compound 74

To a solution of Intermediate Compound 73 (300 mg, 0.77 mmol) in dichloromethane (5 mL) were added triethylamine (0.33 mL, 2.31 mmol) and methanesulfonyl anhydride (175 mg, 1.00 mmol) at 0° C. After stirred at room temperature for 1 hour, the reaction solution was diluted with dichloromethane (50 mL) and washed with distilled water (30 mL). The organic layer was dried over anhydrous sodium sulfate. Filtration and concentration under reduced pressure gave Intermediate Compound 74 (380 mg, crude) which was used without further purification. EI-MS m/z: [M+H]⁺ 467.17.

### Preparation of Intermediate Compound 76

To a solution of Intermediate Compound 75 (450 mg, 0.62 mmol, Intermediate Compound 75 was prepared according to the method described in the International patent publication No. WO 2022/155518 A1) in N,N-dimethylformamide (5 mL) were added cesium carbonate (811 mg, 2.49 mmol) and Intermediate Compound 74 (349 mg, 0.75 mmol) in N,N-dimethylformamide (2 mL). After stirred at room temperature for 3 hours, the reaction solution was concentrated under reduced pressure and diluted with dichloromethane (50 mL) and methanol (10 mL) and washed with distilled water (30 mL). The organic layer was dried over anhydrous sodium sulfate. After filtration and concentration under reduced pressure, the resulting residue was purified by column chromatography to afford Intermediate Compound 76 (468 mg, 68%). EI-MS m/z: [M+H]⁺ 1093.72.

### Preparation of Intermediate Compound 77

To a solution of Intermediate Compound 76 (468 mg, 0.43 mmol) in tetrahydrofuran (10 mL) was added tetrabutylammonium fluoride (1.0 M tetrahydrofuran solution, 2.1 mL, 2.14 mmol). After stirred under reflux for 5 hours, the reaction solution was concentrated under reduced pressure and the resulting residue was purified by reversed-phase column chromatography, which afforded Intermediate Compound 77 (256 mg, 63%). EI-MS m/z [M+H]⁺ 950.12.

### Preparation of Intermediate Compound 78

To a solution of Intermediate Compound 77 (256 mg, 0.27 mmol) in N,N-dimethylformamide (3 mL) were added N,N-bis(t-butoxycarbonyl)-1H-pyrazole-1-carboxamidine (126 mg, 0.40 mmol) and triethylamine (0.11 mL, 0.81 mmol). After stirred at 60° C. for 17 hours, the reaction solution was concentrated under reduced pressure. The resulting residue was purified by column chromatography to afford Intermediate Compound 78 (103 mg, 32%). EI-MS m/z: [M+H]⁺ 1192.17.

### Preparation of Intermediate Compound 79

To a solution of Intermediate Compound 78 (103 mg, 0.09 mmol) in dichloromethane (3 mL) was added trifluoroacetic acid (1 mL) at 0° C. under nitrogen. The reaction solution was stirred at room temperature for 2 hours. After concentration, the resulting residue was purified by HPLC to afford Intermediate Compound 79 (48 mg, 45%)

¹H-NMR (400 MHz, CD₃OD) δ 7.29 (d, J=1.4 Hz, 1H), 7.26 (d, J=1.4 Hz, 1H), 6.59 (d, J=0.6 Hz, 1H), 6.56 (d, J=0.6 Hz, 1H), 5.88-5.77 (m, 2H), 5.75-5.69 (m, 2H), 5.02 (d, J=3.3 Hz, 4H), 4.64-4.50 (m, 6H), 3.94 (d, J=4.2 Hz, 2H), 3.75 (s, 3H), 3.42-3.32 (m, 4H), 3.01-2.93 (m, 2H), 2.24-2.17 (m, 6H), 1.97 (p, J=7.8 Hz, 2H), 1.34 (dt, J=14.6, 7.1 Hz, 6H). EI-MS m/z: [M+H]⁺ 891.09.

### Example 13: Preparation of Intermediate Compound 89

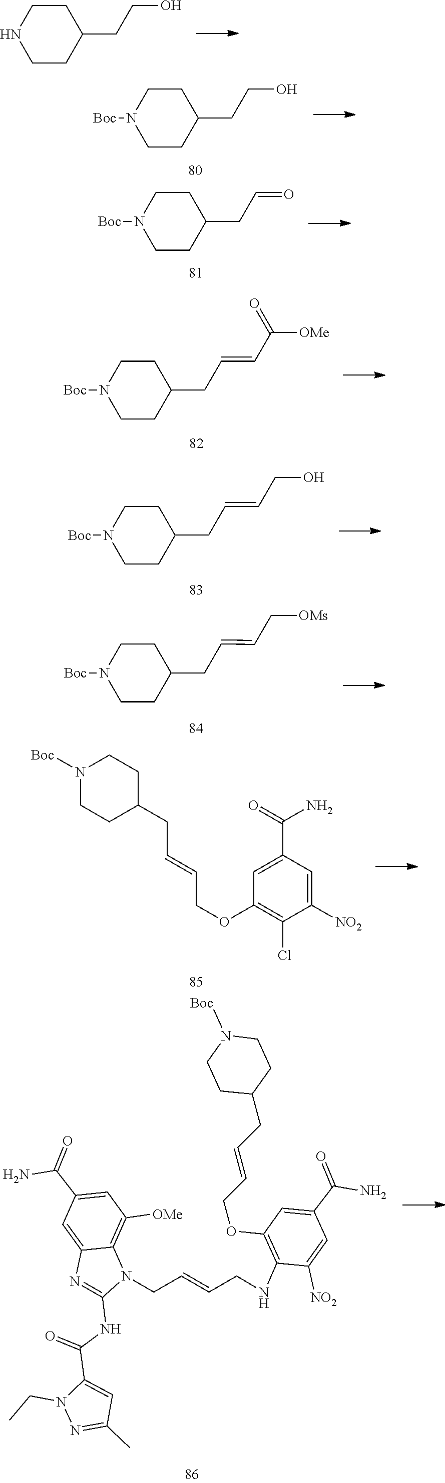

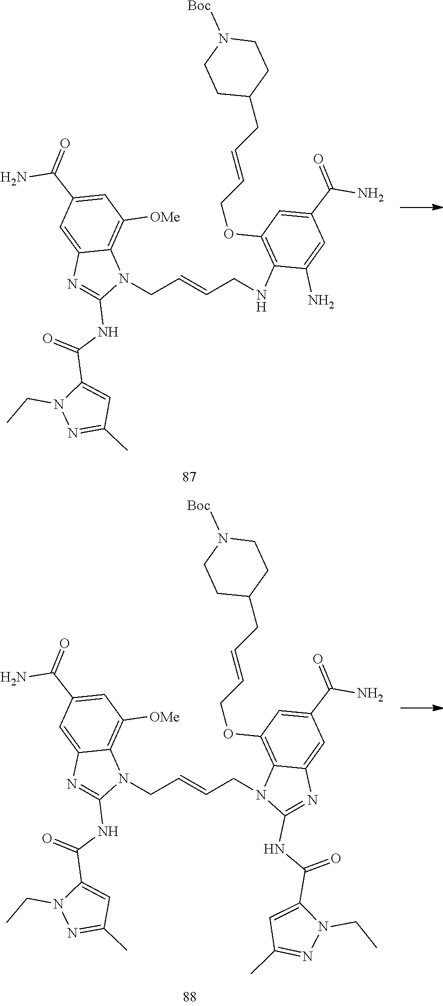 [SMILES: CCn1nc(C)cc1C(=O)Nc1nc2cc(C(N)=O)cc(OC)c2n1C/C=C/CNc1c(N)cc(C(N)=O)cc1OC/C=C/CC1CCN(C(=O)OC(C)(C)C)CC1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CC3CCN(C(=O)OC(C)(C)C)CC3)c21]

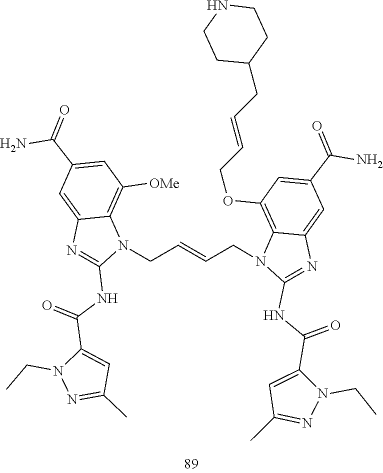 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CC3CCNCC3)c21]

### Preparation of Intermediate Compound 80

To a solution of 4-Piperidinethanol (5 g, 38.7 mmol) in dichloromethane (200 mL) were added triethylamine (8.1 mL, 58.05 mmol) and di-t-butyl dicarbonate (9.78 mL, 42.57 mmol) under nitrogen. The reaction solution was stirred at room temperature for 3 hours and then diluted with ethyl acetate (50 mL) and washed with distilled water (50 mL×2). The organic layer was dried over anhydrous sodium sulfate. After filtration and concentration under reduced pressure, the resulting residue was purified by column chromatography to afford Intermediate Compound 80 (7.25 g, 81.6%).

¹H-NMR (CDCl₃) δ: 1.04-1.10 (m, 2H), 1.48 (s, 9H), 1.49-1.55 (m, 3H), 1.60-1.66 (m, 2H), 2.27 (s, 1H), 2.64 (t, J=8.0 Hz, 2H), 3.64 (t, J=8.0 Hz, 2H), 4.03-4.08 (m, 2H). EI-MS m/z: [M+Na]⁺252.26.

### Preparation of Intermediate Compound 81

To a solution of Dimethyl sulfoxide (0.93 mL, 13.08 mmol) in dichloromethane (20 mL) was slowly added oxalyl chloride (0.34 mL, 3.93 mmol) at −78° C. under nitrogen. After stirred for 30 minutes, the reaction solution was added Intermediate Compound 80 (1 g, 4.36 mmol) in dichloromethane (5 mL). The reaction solution was stirred at −50° C. for 2 hours. The reaction solution was added triethylamine (1.8 mL, 13.1 mmol). After raised to 0° C. stirred for 30 minutes, the reaction solution was diluted with ethyl acetate (50 mL), washed with distilled water (30 mL). The organic layer was dried over anhydrous sodium sulfate. After filtration and concentration under reduced pressure, the resulting residue was purified by column chromatography to afford Intermediate Compound 81 (990 mg, 99%).

¹H-NMR (300 MHz, CDCl₃), δ (ppm): 9.78 (br s, 1H), 4.08 (br d, 2H), 2.74 (br t, 2H), 2.39 (d, 2H), 2.12-1.89 (m, 1H), 1.79-1.64 (m, 2H), 1.45 (s, 9H), 1.26-1.10 (m, 2H). EI-MS m/z: [M+H]⁺ 228.23.

### Preparation of Intermediate Compound 82

To a solution of lithium chloride (17.2 g, 40.65 mmol) in acetonitrile (40 mL) was added triethylphosphonoacetate (7.33 mL, 50.81 mmol) at room temperature under nitrogen. After stirred for 5 minutes, the reaction solution was added Triethylamine (5.67 mL 40.65 mmol) at room temperature for 10 minutes. Compound 81 (7.7 g, 33.88 mmol) in acetonitrile (60 mL) was added to the reaction solution. After stirred at room temperature for 17 hours, the reaction solution was diluted with ethyl acetate (150 mL), washed with distilled water (150 mL). The organic layer was dried over anhydrous magnesium sulfate. After filtration and concentration under reduced pressure, the resulting residue was purified by column chromatography to afford Intermediate Compound 82 (5.59 g, 58.2%).

¹H-NMR (400 MHz, CDCl₃), δ (ppm): 6.96-6.88 (m, 1H), 5.85-5.811 (m, 1H), 4.08 (s, 2H), 3.73 (s, 3H), 2.07-2.64 (m, 2H), 2.17-2.13 (m, 2H), 1.67 (s, 2H), 1.59-1.55 (m, 1H), 1.54 (s, 9H), 1.18-1.11 (m, 2H). EI-MS m/z: [M+H]⁺ 284.01.

### Preparation of Intermediate Compound 83

To a solution of Intermediate Compound 82 (2.5 g, 8.82 mmol) in dichloromethane (30 mL) was added diisobutylaluminium hydride (1.0 M cyclohexane solution, 19 mL, 19.00 mmol) at −78° C. under nitrogen. After stirred at −78° C. for 3 hours, the reaction solution was added methanol (100 mL). The reaction solution was filtered through Celite and washed with methanol. The filtrate was removed under reduced pressure and used without purification to afford Intermediate Compound 83 (1.91 g, 84.8%).

¹H-NMR (400 MHz, CDCl₃): δ=5.70-5.65 (m, 2H), 4.18-4.01 (m, 4H), 2.69 (t, 2H, J=12.6 Hz), 2.02 (t, 2H, J=5.6 Hz), 1.72-1.62 (m, 3H), 1.47 (s, 9H), 1.11 (qd, 2H, J=12.2 Hz, 3.9 Hz). EI-MS m/z: [M+H]⁺ 256.06.

### Preparation of Intermediate Compound 84

To a solution of Intermediate Compound 83 (1.91 g, 7.48 mmol) in dichloromethane (20 mL) were added triethylamine (1.6 mL, 11.2 mmol) and methanesulfonyl chloride (0.87 mL, 11.2 mmol) at 0° C. After stirred at room temperature for 3 hours, the reaction mixture diluted with ethyl acetate (20 mL) and washed with distilled water (30 mL). The organic layer was dried over anhydrous sodium sulfate. After filtration, the filtrate was removed under reduced pressure and used without purification to afford Intermediate Compound 84 (2.59 g, crude).

### Preparation of Intermediate Compound 85

To a solution of Intermediate Compound 5 (1 g, 4.62 mmol) in N,N-dimethylformamide (15 mL) were added potassium carbonate (766 mg, 5.54 mmol) and compound 84 (2.3 g, 6.94 mmol). After stirred at room temperature for 14 hours, the reaction mixture diluted with ethyl acetate (20 mL) and washed with distilled water (30 mL). The organic layer was dried over anhydrous sodium sulfate and filtered. The filtrate was concentrated under reduced pressure. The residue was purified by column chromatography to afford Intermediate Compound 85 (1.18 g, 56.3%).

¹H-NMR (400 MHz, CDCl₃): δ=7.82-7.72, 5.83-5.69 (m, 2H), 4.80-4.71 (m, 2H), 4.04-4.03 (m, 2H), 2.65 (s, 2H), 2.05-1.93 (m, 1H), 1.07-1.04 (m, 2H). EI-MS m/z: [M+H]⁺ 454.17.

### Preparation of Intermediate Compound 86

To a solution of Intermediate Compound 85 (550 mg, 1.21 mmol) in n-butanol (5 mL) were added Intermediate Compound 9 (1.17 g, 2.42 mmol) and N,N-diisopropylethylamine (1.05 mL, 6.06 mmol). After stirred at 0° C. for 5 minutes, the reaction solution was heated to 120° C. and stirred for 24 hours and then cooled to room temperature. The reaction mixture was concentrated under reduced pressure. The concentrated reaction product was purified by column chromatography to afford Intermediate Compound 86 (157 mg, 15.6%). EI-MS m/z: [M+H]⁺ 829.22.

### Preparation of Intermediate Compound 87

To a solution of Intermediate Compound 86 (157 mg, 0.19 mmol) in methanol (3 mL) were added ammonia solution (28-30% ammonia, 0.5 mL, 4.74 mmol) and sodium hydrosulfite (Na₂S₂O₄, 330 mg, 1.89 mmol). After stirred at room temperature for 1 hour, the reaction solution was passed through Celite filter and washed with methanol. After filtration, the filtrate was removed under reduced pressure and used without purification to afford Intermediate Compound 87 (151 mg, crude), which was used without further purification.

### Preparation of Intermediate Compound 88

To a solution of Intermediate Compound 87 (151 mg, 0.19 mmol) in N,N-dimethylformamide (2 mL) was added compound 2 (40 mg, 0.21 mmol) at 0° C. The reaction solution was stirred for 30 minutes and then N-(3-dimethylaminopropyl)-N′-ethylcarbodiimide (0.04 mL, 0.24 mmol) and triethylamine (0.11 mL, 0.76 mmol) were added and the reaction solution was stirred at room temperature for 15 hours. The reaction solution was concentrated under reduced pressure and purified by column chromatography to afford Intermediate Compound 88 (100 mg, 54%). EI-MS m/z: [M+H]⁺ 961.13.

### Preparation of Intermediate Compound 89

To a solution of Intermediate Compound 88 (20 mg, 0.02 mmol) in dichloromethane (2 mL) was added trifluoroacetic acid (0.4 mL) at 0° C. under nitrogen. The reaction solution was stirred at room temperature for 0.5 hours. After concentration, the resulting residue was purified by HPLC to afford Intermediate Compound 89 (4.8 mg, 27%). EI-MS m/z: [M+H]⁺ 861.30.

### Example 14: Preparation of Intermediate Compound 95

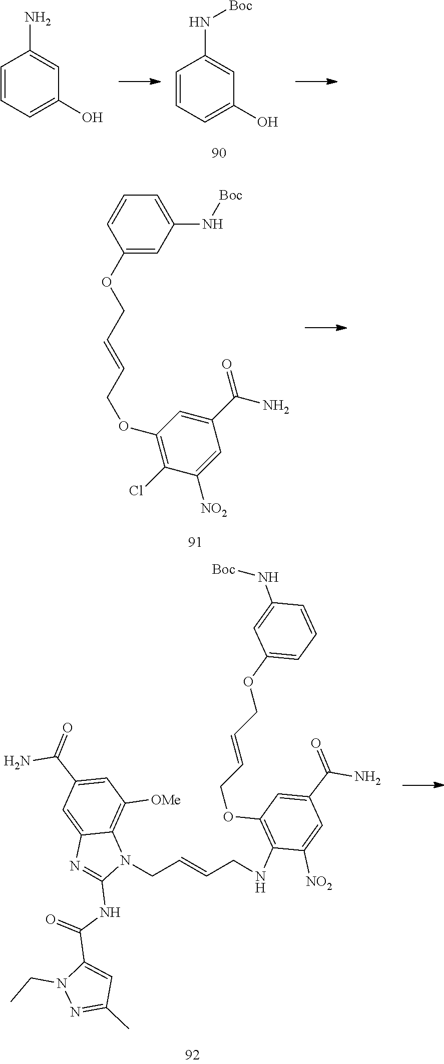 [SMILES: Nc1cccc(O)c1 ; CC(C)(C)OC(=O)Nc1cccc(O)c1 ; CC(C)(C)OC(=O)Nc1cccc(OC/C=C/COc2cc(C(N)=O)cc([N+](=O)[O-])c2Cl)c1 ; CCn1nc(C)cc1C(=O)Nc1nc2cc(C(N)=O)cc(OC)c2n1C/C=C/CNc1c(OC/C=C/COc2cccc(NC(=O)OC(C)(C)C)c2)cc(C(N)=O)cc1[N+](=O)[O-]]

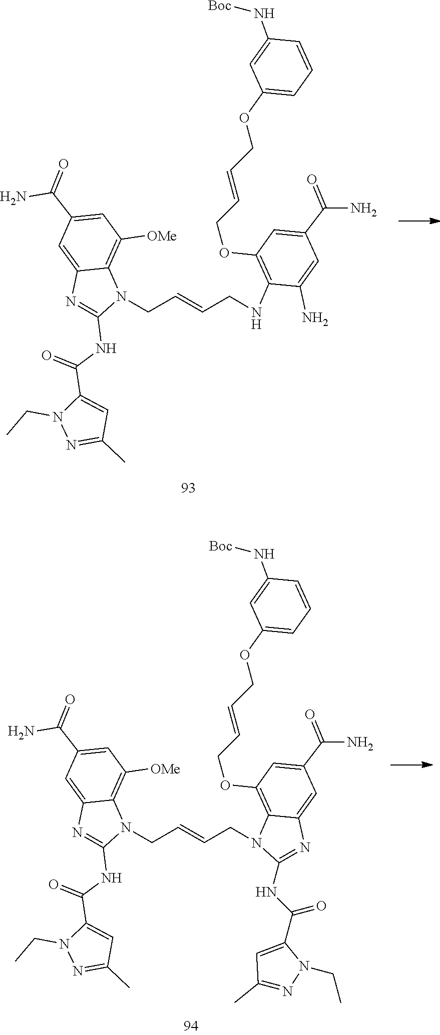 [SMILES: CCn1nc(C)cc1C(=O)Nc1nc2cc(C(N)=O)cc(OC)c2n1C/C=C/CNc1c(N)cc(C(N)=O)cc1OC/C=C/COc1cccc(NC(=O)OC(C)(C)C)c1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/COc3cccc(NC(=O)OC(C)(C)C)c3)c21]

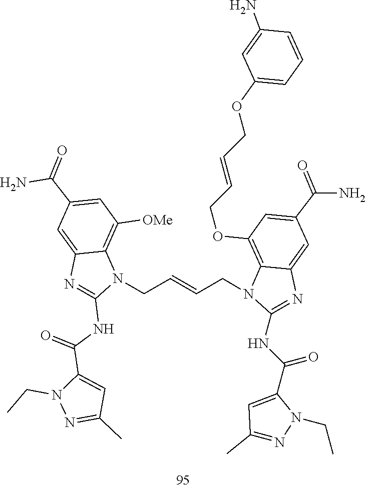 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/COc3cccc(N)c3)c21]

### Preparation of Intermediate Compound 90

To a solution of 3-Aminophenol (1 g, 9.16 mmol) in tetrahydrofuran (10 mL) was added di-t-butyl dicarbonate (2.52 mL, 10.99 mmol). After stirred for 16 hours at room temperature, the reaction solution was diluted with ethyl acetate (50 mL) and washed with saturated aqueous ammonium chloride solution (50×2 mL). The reaction solution was dried over anhydrous sodium sulfate. After filtration, the solvent was removed under reduced pressure and used without purification to afford Compound 90 (1.8 g, 93%).

¹H-NMR (400 MHz, CDCl₃) δ 9.24 (s, 1H), 9.18 (s, 1H), 6.88 (m, 2H), 6.83 (m, 1H), 6.35 (m, 1H), 1.46 (s, 9H). EI-MS m/z: [M+H]⁺ 209.10.

### Preparation of Intermediate Compound 91

To a solution of Intermediate Compound 90 (300 mg, 1.43 mmol) in N,N-dimethylformamide (5 mL) were added cesium carbonate (560 mg, 1.72 mmol) and Intermediate Compound 5 (551 mg, 1.57 mmol) at 0° C. under nitrogen. After stirred for 2 hours, the reaction solution was diluted with ethyl acetate (30 mL) and washed with distilled water (15 mL×2) and brine (15 mL). The organic layer was dried over anhydrous sodium sulfate and filtered and concentrated. The concentrate was solidified by added dichloromethane and diethyl ether, then filtered and dried to afford Intermediate Compound 91 (474 mg, 69%), which was used without further purification.

¹H-NMR (400 MHz, CDCl₃) δ 9.29 (s, 1H), 8.26 (s, 1H), 8.06 (s, 1H), 7.90 (s, 1H), 7.75 (s, 1H), 7.12 (m, 2H), 7.10 (d, 1H), 6.56 (m, 1H), 6.14 (m, 2H), 4.87 (d, J=3.6 Hz, 2H), 4.57 (d, J=3.2 Hz, 2H), 1.46 (s, 9H). EI-MS m/z: [M+H]⁺ 478.00.

### Preparation of Intermediate Compound 92

To a solution of Intermediate Compound 9 (607 mg, 1.47 mmol) and Intermediate Compound 91 (470 mg, 0.98 mmol) in n-butanol (5 mL) were added diisopropylethylamine (0.68 mL, 3.93 mmol) at room temperature. After heated to 120° C. for 24 hours, the reaction solution was cooled to room temperature. The reaction solution was diluted with dichloromethane (100 mL) and methanol (20 mL) and washed with distilled water (50 mL). The organic layer was dried over anhydrous sodium sulfate and filtered and concentrated to afford Intermediate Compound 92 (100 mg, 12%), which was used without further purification. EI-MS m/z: [M+H]⁺ 853.30.

### Preparation of Intermediate Compound 93

To a solution of Intermediate Compound 92 (100 mg, 0.11 mmol) in methanol (5 mL) were added ammonia solution (28-30% ammonia, 0.209 mL) and sodium hydrosulfite (Na₂S₂O₄, 204 mg, 1.172 mmol) under nitrogen. After stirred at room temperature for 1 hour, the reaction solution was diluted with methanol (50 mL) and then filtered. The filtrate was concentrated under reduced pressure. The reaction mixture was diluted with dichloromethane (100 mL) and methanol (20 mL) and washed with distilled water (50 mL). The organic layer was dried over anhydrous sodium sulfate and filtered and concentrated to afford Intermediate Compound 93 (100 mg, crude), which was used without further purification. EI-MS m/z: [M+H]⁺ 823.36.

### Preparation of Intermediate Compound 94

To a solution of Intermediate Compound 93 (100 mg, 0.12 mmol, crude) in N,N-dimethylformamide (2 mL) were added Intermediate Compound 2 (26 mg, 0.13 mmol) in N,N-dimethylformamide (1 mL) at 0° C. under nitrogen. After stirred at room temperature for 1 hour. The reaction mixture was added N-(3-dimethylaminopropyl)-N′-ethylcarbodiimide hydrochloride (28 mg, 0.18 mmol) and triethylamine (0.05 mL, 0.36 mmol) After stirred at room temperature for 13 hours, the reaction solution was concentrated under reduced pressure and solidified by dichloromethane and diethyl ether, then filtered and dried to afford Intermediate Compound 94 (54 mg, 53%). EI-MS m/z: [M+H]⁺ 984.84.

### Preparation of Intermediate Compound 95

To a solution of Intermediate Compound 94 (50 mg, 0.061 mmol) in dichloromethane (2 mL) was added trifluoroacetic acid (0.4 mL) at 0° C. under nitrogen. After stirred at room temperature for 40 minutes, the reaction solution was concentrated and purified by HPLC to afford Intermediate Compound 95 (1.7 mg, 3%). EI-MS m/z: [M+H]⁺ 884.37.

### Example 15: Preparation of Intermediate Compound 104

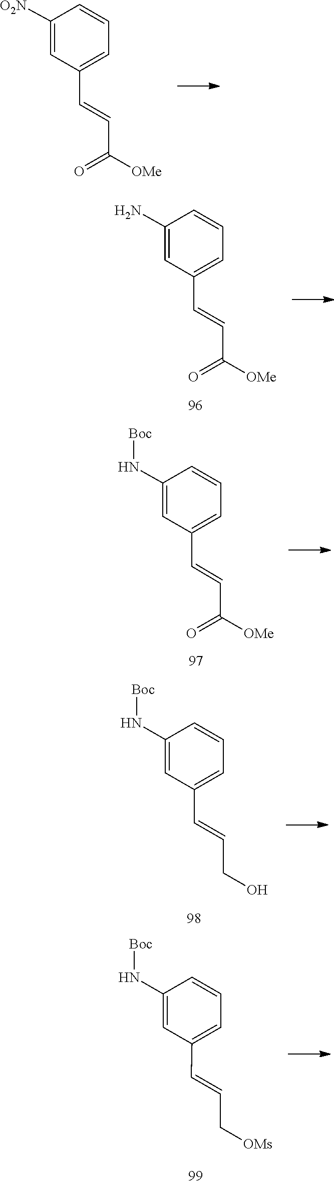

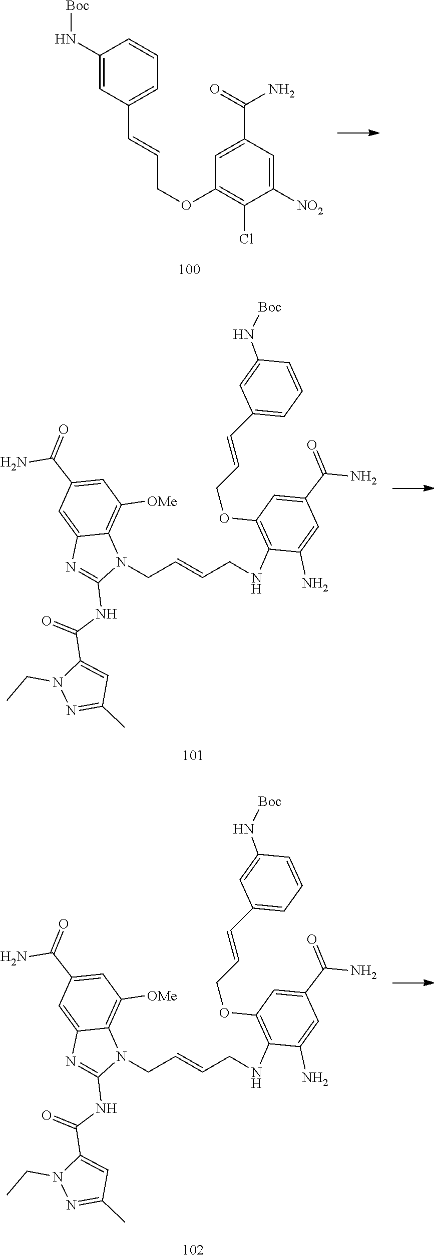 [SMILES: CC(C)(C)OC(=O)Nc1cccc(/C=C/COc2cc(C(N)=O)cc([N+](=O)[O-])c2Cl)c1 ; CCn1nc(C)cc1C(=O)Nc1nc2cc(C(N)=O)cc(OC)c2n1C/C=C/CNc1c(N)cc(C(N)=O)cc1OC/C=C/c1cccc(NC(=O)OC(C)(C)C)c1 ; CCn1nc(C)cc1C(=O)Nc1nc2cc(C(N)=O)cc(OC)c2n1C/C=C/CNc1c(N)cc(C(N)=O)cc1OC/C=C/c1cccc(NC(=O)OC(C)(C)C)c1]

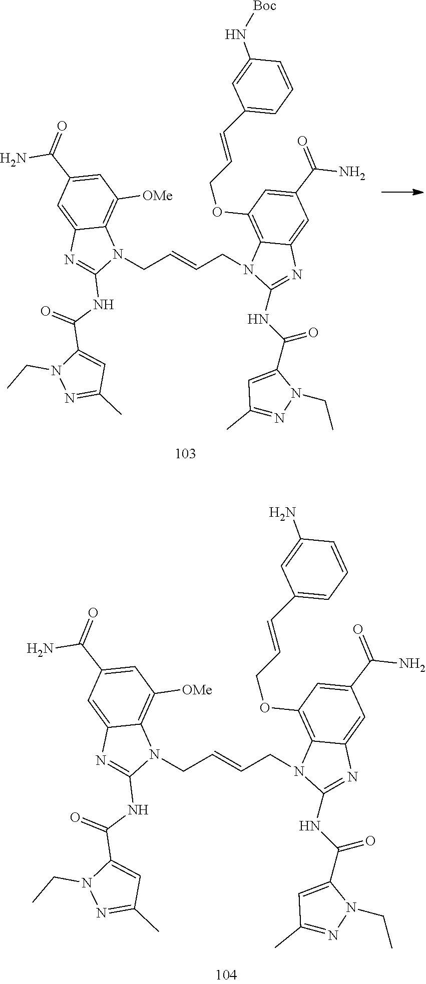 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/c3cccc(NC(=O)OC(C)(C)C)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/c3cccc(N)c3)c21]

### Preparation of Intermediate Compound 96

To a solution of methyl 3-nitrocinnamate (3.5 g, 16.89 mmol) in methanol (20 mL) and distilled water (5 mL) were added conc. hydrochloric acid (0.25 mL) and iron powder (9 g, 161.15 mmol). After heated to reflux with stirring for 17 hours, the reaction solution was filtered through Celite and concentrated under reduced pressure to afford Intermediate Compound 96 (2.7 g, crude) without purification.

¹H-NMR (400 MHz, DMSO-d₆): δ 7.48 (d, J=15.8 Hz, 1H), 7.06 (t, J=7.8 Hz, 1H), 6.85-6.79 (m, 2H), 6.65-6.61 (m, 1H), 6.41 (d, J=15.8 Hz, 1H), 5.19 (s, 2H), 3.71 (s, 3H).

### Preparation of Intermediate Compound 97

To a solution of Intermediate Compound 96 (2.7 g, 15.24 mmol) was dissolved in 1,4-dioxane (10 mL were added di-t-butyl dicarbonate (3.85 mL, 16.76 mmol) and saturated aqueous sodium bicarbonate solution (3.2 g, 38.09 mmol) dissolved in water (50 mL). After stirred at room temperature for 21 hours, the reaction mixture was diluted with ethyl acetate (50 mL) and washed with distilled water (50 mL×2). The reaction solution was dried over anhydrous sodium sulfate. After filtration, the filtrate was concentrated under reduced pressure and purified by column chromatography to afford Intermediate Compound 97 (3 g, 71%).

¹H-NMR (CD₃OD, 400 MHz) δ 7.65 (s, 1H), 7.62 (d, 1H, J=16.1 Hz), 7.43 (d, 1H, J=7.6 Hz), 7.28 (t, 1H, J=7.6 Hz), 7.20 (d, 1H, J=7.6 Hz), 6.47 (d, 1H, J=16.1 Hz), 3.76 (s, 3H), 1.51 (s, 9H).

### Preparation of Intermediate Compound 98

To a solution of Intermediate Compound 97 (1.3 g, 4.69 mmol) in dichloromethane (20 mL) were added diisobutylaluminum hydride (1.0 M in cyclohexane, 19 mL, 18.75 mmol) at −78° C. under nitrogen. After stirred at −78° C. for 3 hours, the reaction solution was added methanol (100 mL) at room temperature. The reaction solution was filtered through Celite. The filtrate was concentrated under reduced pressure. The residue was purified by column chromatography to afford Intermediate Compound 98 (815 mg, 70%). EI-MS m/z: [M+Na]⁺ 272.05.

### Preparation of Intermediate Compound 99

To a solution of Intermediate Compound 98 (800 mg, 3.21 mmol) in dichloromethane (16 mL) were added triethylamine (0.7 mL, 4.81 mmol) and methanesulfonyl chloride (0.3 mL, 3.53 mmol) at 0° C. After stirred at room temperature for 3 hours, the reaction solution diluted with ethyl acetate (20 mL) and washed with distilled water (30 mL). The organic layer was dried over anhydrous magnesium sulfate and filtered. The filtrate was concentrated under reduced pressure to afford Intermediate Compound 99 (1 g, 95%), which was used without further purification.

### Preparation of Intermediate Compound 100

To a solution of Intermediate Compound 4 (762 mg, 3.52 mmol) in N,N-dimethylformamide (15 mL) were added potassium carbonate (663 mg, 4.8 mmol) and compound 99 (1.05 g, 3.2 mmol). After stirred at 50° C. for 15 hours, the reaction solution diluted with ethyl acetate (20 mL) and washed with distilled water (30 mL). The organic layer was dried over anhydrous sodium sulfate and filtered. The filtrate was concentrated under reduced pressure to afford Intermediate Compound 100 (815 mg, 57%), which was used without further purification. EI-MS m/z: [M+H]⁺ 448.25.

### Preparation of Intermediate Compound 101

To a solution of Intermediate Compound 100 (500 mg, 1.12 mmol) in n-butanol (6 mL) were added compound 9 (811 mg, 1.68 mmol) and N,N-diisopropylethylamine (0.8 mL, 4.47 mmol). After stirred at 0° C. for 5 minutes, the reaction mixture was heated to 120° C. and stirred for 23 hours. The reaction solution was cooled to room temperature and concentrated under reduced pressure. The resulting residue was purified by column chromatography to afford Intermediate Compound 101(316 mg, 34%). EI-MS m/z: [M+H]⁺ 823.42.

### Preparation of Intermediate Compound 102

To a solution of Intermediate Compound 101 (316 mg, 0.38 mmol) in methanol (6 mL) were added aqueous ammonia solution (28-30% ammonia, 0.7 mL, 9.6 mmol) and sodium hydrosulfite (Na₂S₂O₄, 668 mg, 3.84 mmol). After stirred at room temperature for 1 hour, the precipitate was filtered through Celite and washed with methanol. The filtrate was concentrated under reduced pressure and obtained Intermediate Compound 102 (304 mg, crude), which was used without further purification. EI-MS m/z: [M+H]⁺ 793.51.

### Preparation of Intermediate Compound 103

To a solution of Intermediate Compound 102 (304 mg, 0.38 mmol) in N,N-dimethylformamide (2 mL) was added compound 2 (82 mg, 0.42 mmol) at 0° C. After stirred for 30 minutes, the reaction solution was added N-(3-dimethylaminopropyl)-N′-ethylcarbodiimide (0.08 mL, 0.48 mmol) and triethylamine (0.21 mL, 1.54 mmol) at room temperature for 15 hours. The reaction solution was concentrated under reduced pressure. The resulting residue was purified by column chromatography to afford Intermediate Compound 103 (12 mg, 3%). EI-MS m/z: [M+H]⁺ 954.47.

### Preparation of Intermediate Compound 104

To a solution of Intermediate Compound 103 (12 mg, 0.001 mmol) in dichloromethane (1 mL) was added trifluoroacetic acid (0.4 mL) at 0° C. under nitrogen. After stirred at room temperature for 0.5 hours, the reaction solution was concentrated. The resulting residue was purified by HPLC to afford Intermediate Compound 104 (4.3 mg, 40%). EI-MS m/z: [M+H]⁺ 855.46.

### Example 16: Preparation of Intermediate Compound 110

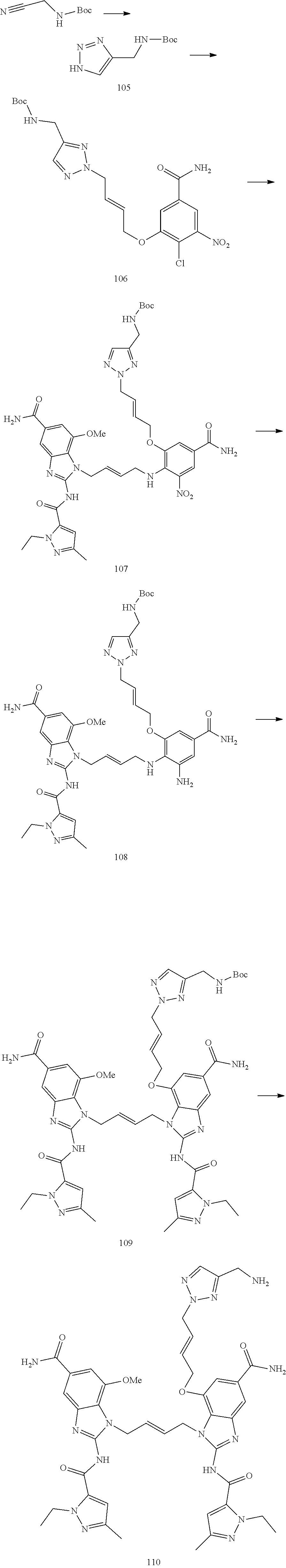

### Preparation of Intermediate Compound 105

To a solution of t-butyl prop-2-en-1-ylcarbamate (1.0 g, 6.44 mmol) in N,N-dimethylformamide (9 mL) and methanol (1 mL) were added trimethylsilylazide (1.27 mL, 9.66 mmol) and copper(I) iodide (613 mg, 3.22 mmol). After stirred at 90° C. for 18 hours, the reaction solution was diluted with ethyl acetate (200 mL) and washed with saturated aqueous ammonium chloride solution (50 mL×2) and dried over anhydrous sodium sulfate. After filtration and concentration under reduced pressure, the resulting residue was purified by column chromatography to afford Intermediate Compound 105 (373 mg, 290%).

¹H-NMR (400 MHz, CDCl₃) δ 7.65 (s, 1H), 5.09 (s, 1H), 4.43 (d, J=6.0 Hz, 2H), 1.46 (s, 9H).

### Preparation of Intermediate Compound 106

To a solution of Intermediate Compound 105 (122 mg, 0.62 mmol) and compound 5 (315 mg, 0.68 mmol) in N,N-dimethylformamide (55 mL) was added potassium carbonate (102 mg, 0.74 mmol) at room temperature under nitrogen. After stirred for 16 hours, the reaction solution was diluted with ethyl acetate (200 mL) and washed with distilled water (150 mL×2) and brine (150 mL). The reaction solution was dried over anhydrous sodium sulfate. After filtration and concentration under reduced pressure, the resulting residue was purified by column chromatography to afford Intermediate Compound 106 (146 mg, 500%).

¹H-NMR (400 MHz, DMSO) δ 8.27 (s, 1H), 8.06 (s, 1H), 7.86 (d, J=8.3 Hz, 2H), 7.78 (s, 1H), 7.30 (s, 1H), 6.17-6.07 (m, 1H), 5.97-5.93 (m, 1H), 5.07 (d, J=6.0 Hz, 2H), 4.84 (d, J=5.2 Hz, 2H), 4.16 (d, J=5.9 Hz, 2H), 1.38 (s, 9H).

### Preparation of Intermediate Compound 107

To a solution of Intermediate Compound 106 (265 mg, 0.57 mmol) and Intermediate Compound 9 (412 mg, 0.85 mmol) in n-butanol (3 mL) was added N,N-diisopropylethylamine (0.49 mL, 2.84 mmol) at room temperature. After heated to 100° C. and stirred for 21 hours, the reaction solution was cooled to room temperature and diluted with dichloromethane (100 mL) and methanol (20 mL) and washed with distilled water (50 mL). The organic layer was dried over anhydrous sodium sulfate and filtered. The filtrate was concentrated under reduced pressure. The resulting residue was purified by column chromatography to afford Intermediate Compound 107 (352 mg, 73%). EI-MS m/z: [M+H]⁺ 842.42.

### Preparation of Intermediate Compound 108

To a solution of Intermediate Compound 107 (352 mg, 0.42 mmol) in methanol (10 mL) and distilled water (2 mL) were added ammonia solution (28-30% ammonia, 0.6 mL) and sodium hydrosulfite (Na₂S₂O₄, 728 mg, 4.2 mmol) under nitrogen. After stirred at room temperature for 2 hours, the reaction solution was diluted with methanol (50 mL) and then filtered. The filtrate was concentrated under reduced pressure. The reaction mixture was diluted with dichloromethane (100 mL) and methanol (20 mL) and washed with distilled water (50 mL). The organic layer was dried over anhydrous sodium sulfate and filtered and concentrated to afford Intermediate Compound 108 (100 mg, 29%), which was used without further purification. EI-MS m/z: [M+H]⁺ 812.47.

### Preparation of Intermediate Compound 109

To a solution of Intermediate Compound 108 (100 mg, 0.12 mmol) in N,N-dimethylformamide (3 mL) was added compound 2 (29 mg, 0.15 mmol) in N,N-dimethylformamide (1 mL) under nitrogen. After stirred at room temperature for 1 hour, the reaction solution was added N-(3-dimethylaminopropyl)-N′-ethylcarbodiimide (0.03 mL, 0.17 mmol) and triethylamine (0.05 mL, 0.05 mmol) at room temperature for 16 hours. The reaction solution was concentrated under reduced pressure. The resulting residue was purified by column chromatography to afford the Intermediate Compound 109 (80 mg, 66%). EI-MS m/z: [M+H]⁺ 973.54.

### Preparation of Intermediate Compound 110

To a solution of Intermediate Compound 109 (80 mg, 0.08 mmol) in dichloromethane (3 mL) was added trifluoroacetic acid (1 mL) at −78° C. under nitrogen. After stirred at room temperature for 2 hours, the reaction solution was concentrated under reduced pressure. The resulting residue was purified by HPLC to afford Intermediate compound 110 (25 mg, 35%).

¹H-NMR (400 MHz, DMSO) δ 12.84 (s, 1H), 8.19 (s, 2H), 8.04-7.91 (m, 3H), 7.65 (s, 2H), 7.37 (s, 2H), 7.30 (s, 2H), 6.52 (d, J=2.6 Hz, 2H), 5.96-5.79 (m, 3H), 4.95-4.87 (m, 6H), 4.58-4.48 (m, 6H), 4.10 (q, J=5.7 Hz, 2H), 3.70 (s, 3H), 2.10 (d, J=6.5 Hz, 6H), 1.26 (q, J=7.1 Hz, 6H). EI-MS m/z: [M+H]⁺ 873.55.

### Example 17: Preparation of Intermediate Compound 120

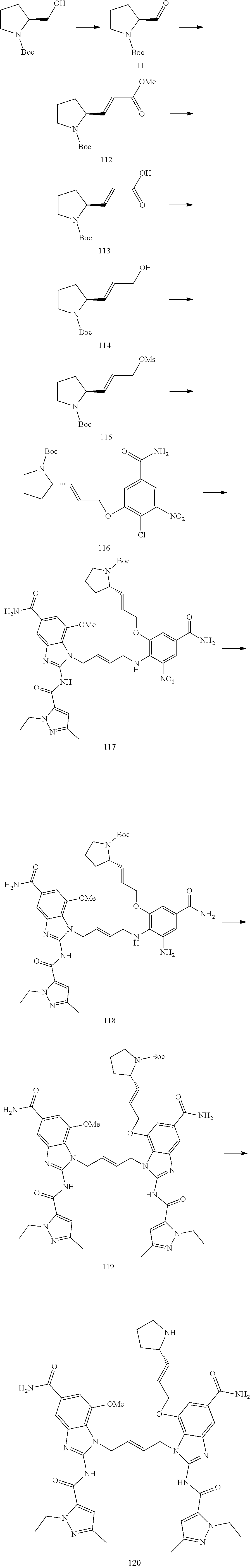

### Preparation of Intermediate Compound 111

To a solution of I t-butyl (S)-2-(hydroxymethyl)pyrrolidine-1-carboxylate (2.2 g, 10.93 mmol) in dichloromethane (20 mL) were added dimethyl sulfoxide (5 mL), triethylamine (9.2 mL, 65.6 mmol) and sulfur trioxide pyridine complex (4.3 g, 27.33 mmol) at 0° C. After stirred at room temperature for 17 hours, the reaction solution was diluted with dichloromethane (100 mL) and washed with distilled water (50 mL) and 0.1 N hydrochloric acid solution (50 mL) and dried over anhydrous sodium sulfate. After filtration and concentration under reduced pressure, the product was purified by column chromatography to afford Intermediate Compound 111 (1.65 g, 75%).

¹H-NMR (400 MHz, CDCl₃) δ 9.60-9.44 (m, 1H), 4.13 (d, J=61.6 Hz, 1H), 3.62-3.40 (m, 2H), 2.23-1.82 (m, 4H), 1.46 (d, J=19.7 Hz, 9H).

### Preparation of Intermediate Compound 112

To a solution of Lithium chloride (421 mg, 9.94 mmol) and trimethyl phosphonoacetate (2.2 g, 12.42 mmol) in acetonitrile (8 mL) was added triethylamine (1.4 mL, mmol) at 0° C. The reaction solution was stirred at room temperature for 10 minutes. The reaction solution was added Intermediate Compound 111 (1.6 g, 8.28 mmol) in acetonitrile (12 mL) After stirred at room temperature for 17 hours, the reaction solution was diluted with diethyl ether (100 mL) and washed with saturated aqueous ammonium chloride solution (50 mL) and brine (50 mL). The organic layer was dried over anhydrous sodium sulfate. After filtration and concentration under reduced pressure, the resulting residue was purified by column chromatography to afford Intermediate Compound 112 (1.43 g, 67%).

¹H-NMR (400 MHz, CDCl₃) δ 6.14 (t, J=10.0 Hz, 1H), 5.74 (d, J=11.4 Hz, 1H), 5.32-5.22 (m, 1H), 3.71 (s, 3H), 3.59-3.34 (m, 2H), 2.31 (d, J=12.9 Hz, 1H), 1.84 (ddt, J=12.5, 8.4, 6.0 Hz, 2H), 1.67 (dt, J=13.2, 6.6 Hz, 1H), 1.42 (d, J=22.5 Hz, 9H).

### Preparation of Intermediate Compound 113

To a solution of Intermediate Compound 112 (700 mg, 2.74 mmol) in tetrahydrofuran (20 mL) was added sodium hydroxide (219 mg, 5.48 mmol) in distilled water (10 mL) at 0° C. After stirred at room temperature for 17 hours, the reaction solution was diluted with ethyl acetate (200 mL) and washed with 1 N hydrochloric acid solution (100 mL). The organic layer was dried over anhydrous sodium sulfate. The filtrate was concentrated under reduced pressure to afford Intermediate Compound 113 (660 mg, 99%), which was used without further purification.

¹H-NMR (400 MHz, CDCl₃) δ 6.91 (d, J=13.0 Hz, 1H), 5.84 (d, J=15.6 Hz, 1H), 4.46 (d, J=56.8 Hz, 1H), 3.45 (s, 2H), 2.10 (s, 1H), 1.87 (q, J=6.6 Hz, 5H), 1.49-1.40 (m, 9H).

### Preparation of Intermediate Compound 114

To a solution of Intermediate Compound 113 (660 mg, 2.74 mmol) in tetrahydrofuran (10 mL) were added isobutyl chloroformate (0.37 mL, 2.87 mmol) and triethylamine (0.46 mL, 3.28 mmol) at −78° C. After stirred at room temperature for 1 hour, the reaction solution was added methanol (5 mL) and sodium borohydride (310 mg, 8.21 mmol). The reaction solution was stirred at room temperature for 2 hours and diluted with ethyl acetate (100 mL) and washed with distilled water (50 mL). The organic layer was dried over anhydrous sodium sulfate and filtered and concentrated under reduced pressure. The resulting residue was purified by column chromatography to afford Intermediate Compound 114 (494 mg, 79%).

¹H-NMR (400 MHz, CDCl₃) δ 5.66 (s, 1H), 4.31 (d, J=39.6 Hz, 1H), 4.14 (d, J=5.0 Hz, 2H), 3.39 (s, 1H), 2.01 (s, 1H), 1.94-1.76 (m, 3H), 1.71 (ddd, J=11.0, 6.7, 3.1 Hz, 4H), 1.45 (d, J=6.8 Hz, 9H).

### Preparation of Intermediate Compound 115

To a solution of Intermediate Compound 114 (494 mg, 2.17 mmol) in dichloromethane (20 mL) were added N-methylmorpholine (0.48 mL, 4.34 mmol) and methanesulfonyl anhydride (416 mg, 2.39 mmol) at −78° C. After stirred at room temperature for 2 hours, the reaction solution was diluted with dichloromethane (100 mL) and washed with distilled water (50 mL). The organic layer was dried over anhydrous sodium sulfate and filtered and concentrated under reduced pressure to afford Intermediate Compound 115 (632 mg, 95%), which was used without further purification.

¹H-NMR (400 MHz, CDCl₃) δ 5.82 (s, 1H), 5.67 (s, 1H), 4.72 (d, J=6.5 Hz, 2H), 4.32 (d, J=39.2 Hz, 1H), 4.17-4.09 (m, 1H), 3.40 (s, 1H), 3.02 (s, 3H), 2.05 (s, 1H), 1.84 (p, J=6.4 Hz, 2H), 1.58 (s, 2H), 1.44 (s, 9H).

### Preparation of Intermediate Compound 116

To a solution of Intermediate Compound 115 (632 mg, 2.07 mmol) and compound 5 (448 mg, 2.07 mmol) in N,N-dimethylformamide (15 mL) was added potassium carbonate (314 mg, 2.27 mmol) at room temperature under nitrogen. After stirred for 16 hours, the reaction solution was diluted with ethyl acetate (200 mL) and washed with distilled water (100 mL×2) and brine (100 mL). The organic layer was dried over anhydrous sodium sulfate and filtered and concentrated under reduced pressure. The resulting residue was purified by column chromatography to afford Intermediate Compound 116 (563 mg, 63%).

¹H-NMR (400 MHz, DMSO) δ 8.25 (s, 1H), 8.04 (d, J=1.9 Hz, 1H), 7.89 (s, 1H), 7.77 (s, 1H), 5.92-5.82 (m, 1H), 5.69-5.62 (m, 1H), 4.83 (d, J=5.9 Hz, 2H), 4.21 (s, 1H), 3.29-3.19 (m, 2H), 1.77 (q, J=6.7 Hz, 2H), 1.65 (s, 1H), 1.33 (d, J=27.7 Hz, 9H).

### Preparation of Intermediate Compound 117

To a solution of Intermediate Compound 116 (300 mg, 0.70 mmol) and Intermediate Compound 9 (511 mg, 0.98 mmol) in n-butanol (4 mL) was added N,N-diisopropylethylamine (0.61 mL, 3.52 mmol) was added at room temperature. After heated to 120° C. and stirred for 20 hours, the reaction solution was cooled to room temperature. The reaction mixture diluted with dichloromethane (100 mL) and methanol (20 mL) and washed with distilled water (50 mL). The organic layer was dried over anhydrous sodium sulfate and filtered and concentrated under reduced pressure. The resulting residue was purified by column chromatography to afford Intermediate Compound 117 (263 mg, 46%). EI-MS m/z: [M+H]⁺ 801.41.

### Preparation of Intermediate Compound 118

To a solution of Intermediate Compound 117 (263 mg, 0.32 mmol) in methanol (10 mL) and distilled water (2 mL) were added ammonia solution (28-30% ammonia, 0.5 mL) and sodium hydrosulfite (Na₂S₂O₄, 572 mg, 3.28 mmol) under nitrogen. After stirred at room temperature for 2 hours, the reaction solution was added methanol (50 mL). The reaction mixture was filtered through Celite and washed with methanol. The filtrate was concentrated and diluted with dichloromethane (100 mL) and washed with distilled water (50 mL). The organic layer was dried over anhydrous sodium sulfate and filtered. The filtrate was concentrated under reduced pressure to afford Intermediate Compound 118 (223 mg, 88%) without purification. EI-MS m/z: [M+H]⁺ 771.43.

### Preparation of Intermediate Compound 119

To a solution of Intermediate Compound 118 (223 mg, 0.29 mmol) in N,N-dimethylformamide (2 mL) were added N-(3-dimethylaminopropyl)-N′-ethylcarbodiimide (0.07 mL, 0.43 mmol) and triethylamine (0.12 mL, 0.87 mmol) under nitrogen. After stirred at room temperature for 1 hour, the reaction solution was added Intermediate Compound 2 (47 mg, 0.24 mmol) in N,N-dimethylformamide (1 mL). After stirred at room temperature for 16 hours, the reaction solution was concentrated under reduced pressure. The resulting residue was purified by column chromatography to afford Intermediate Compound 119 (99 mg, 36%). EI-MS m/z: [M+H]⁺ 932.48.

### Preparation of Intermediate Compound 120

To a solution of Intermediate Compound 119 (99 mg, 0.11 mmol) in dichloromethane (1.6 mL) was added trifluoroacetic acid (0.4 mL) at −78° C. under nitrogen. After stirred at room temperature for 1 hour, the reaction solution was concentrated under reduced pressure. The resulting residue was purified by HPLC to afford Intermediate compound 120 (56 mg, 64%).

### Example 18: Preparation of Intermediate Compound 127

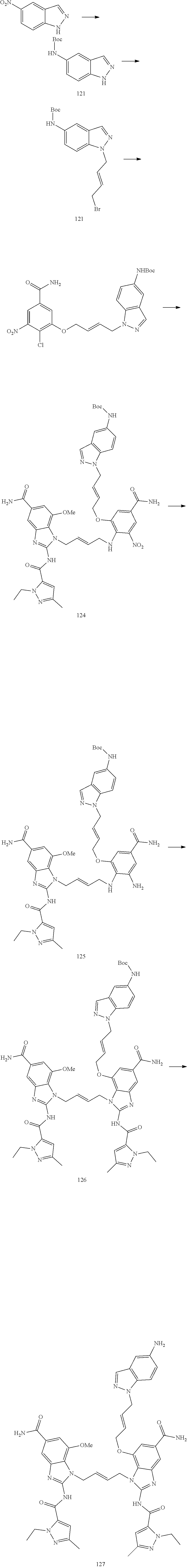

### Preparation of Intermediate Compound 121

To a solution of 5-nitroindole (1 g, 6.13 mmol) in methanol (15 mL) was added palladium/charcoal (85 mg). After stirred at room temperature under hydrogen balloon for 3 hours, the reaction solution was passed through Celite. The filtrate was concentrated under reduced pressure. The concentrated filtrate was dissolved in N,N-dimethylformamide (15 mL) at room temperature and then di-t-butyl dicarbonate (1.3 g, 6.13 mmol) and diisopropylethylamine (0.79 g, 6.13 mmol) were added. After stirred at room temperature for 5 hours, the reaction mixture diluted with ethyl acetate (50 mL) and washed with saturated aqueous ammonium chloride solution (50 mL×2) and dried over anhydrous sodium sulfate. The reaction solution was filtered and concentrated under reduced pressure to afford Intermediate Compound 121 (1.15 g, 80%), which was used without further purification.

¹H-NMR (400 MHz, DMSO-d₆) δ 12.88 (s, 1H), 9.25 (s, 1H), 7.96 (s, 1H), 7.87 (s, 1H), 7.42 (d, J=8.4 Hz, 1H), 7.35 (d, J=8.8 Hz, 1H), 1.48 (s, 9H).

### Preparation of Intermediate Compound 122

To a solution of Intermediate Compound 121 (1 g, 4.28 mmol) in N,N-dimethylformamide (40 mL) were added potassium carbonate (711 mg, 5.14 mmol) and trans-1,4-dibromo-2-butene (2.75 g, 12.86 mmol) at room temperature. After stirred at 60° C. for 18 hours for 6 hours. The reaction solution was diluted with ethyl acetate (50 mL) and washed with distilled water (50 mL×2) and dried over anhydrous sodium sulfate. The reaction solution was filtered and concentrated under reduced pressure. The resulting residue was purified by column chromatography to afford Intermediate Compound 122 (372 mg, 23%).

¹H-NMR (400 MHz, DMSO) δ 9.21 (s, 1H), 8.19 (s, 1H), 7.85 (s, 1H), 7.49 (d, J=8.9 Hz, 1H), 7.21 (d, J=8.5 Hz, 1H), 6.14 (dd, J=15.1, 7.1 Hz, 1H), 5.88 (q, J=7.5 Hz, 1H), 5.05 (d, J=6.0 Hz, 2H), 4.23 (d, J=6.8 Hz, 1H), 4.16 (d, J=7.3 Hz, 2H), 1.48 (s, 9H).

### Preparation of Intermediate Compound 123

To a solution of Intermediate Compound 122 (369 mg, 1.0 mmol) in N,N-dimethylformamide (4 mL) were added 4-chloro-3-hydroxy-5-nitrobenzamide (182 mg, 0.84 mmol) and potassium carbonate (174 mg, 1.26 mmol) under nitrogen. After stirred at 50° C. for 5 hours, the reaction solution was diluted with ethyl acetate (50 mL) and washed with distilled water (50 mL×2). The organic layer was dried over anhydrous sodium sulfate. The filtrate was concentrated under reduced pressure. The resulting residue was purified by column chromatography to afford Intermediate Compound 123 (312 mg, 74%).

¹H-NMR (400 MHz, DMSO-d₆) δ 9.23 (s, 1H), 8.24 (d, J=17.5 Hz, 2H), 8.06 (s, 1H), 7.87 (d, J=13.6 Hz, 2H), 7.78 (s, 1H), 7.50 (d, J=9.2 Hz, 1H), 7.22 (d, J=9.2 Hz, 1H), 6.21 (dt, J=15.8, 6.2 Hz, 1H), 6.01-5.90 (m, 1H), 5.10 (d, J=6.1 Hz, 2H), 4.86 (d, J=5.3 Hz, 2H), 1.48 (s, 9H). EI-MS m/z: [M+H]⁺ 502.31.

### Preparation of Intermediate Compound 124

To a solution of Intermediate Compound 123 (310 mg, 0.62 mmol) and Intermediate Compound 9 (406 mg, 0.98 mmol) in n-butanol (6 mL) was added diisopropylethylamine (0.21 mL, 1.24 mmol) at room temperature. After heated to 120° C. and stirred for 20 hours, the reaction solution was cooled to room temperature. The reaction mixture diluted with dichloromethane (100 mL) and methanol (20 mL) and washed with distilled water (50 mL). The organic layer was dried over anhydrous sodium sulfate and filtered. The filtrate was concentrated under reduced pressure. After dilution with dichloromethane and diethyl ether, the obtained solid was filtered. The solid was dried to afford 124 (crude), which was used without further purification. EI-MS m/z: [M+H]⁺ 877.59.

### Preparation of Intermediate Compound 125

To a solution of Intermediate Compound 124 (0.20 mmol, crude) in methanol (7 mL) and distilled water (1 mL) were added ammonia solution (28-30% ammonia, 0.2 mL) and sodium hydrosulfite (Na₂S₂O₄, 355 mg, 2.04 mmol). After stirred at room temperature for 1 hour, The reaction mixture was diluted with methanol (50 mL) and filtered. The filtrate was concentrated and diluted with acetonitrile (10 mL), and the obtained solid was filtered. The solid was dried to afford Intermediate Compound 125 (crude), which was used without further purification. EI-MS m/z: [M+H]⁺ 847.58.

### Preparation of Intermediate Compound 126

To a solution of Intermediate Compound 125 (0.20 mmol, crude) in N,N-dimethylformamide (4 mL) was added Intermediate Compound 2 (47 mg, 0.24 mmol) in N,N-dimethylformamide (1 mL) under nitrogen. After stirred at room temperature for 1 hour, the reaction solution was added N-(3-dimethylaminopropyl)-N′-ethylcarbodiimide (47 mg, 0.30 mmol) and triethylamine (0.72 mL, 0.61 mmol) at room temperature for 16 hours. The reaction solution was concentrated under reduced pressure. The resulting residue was purified by column chromatography to afford Intermediate Compound 126 (83 mg, 40%). EI-MS m/z: [M+H]⁺ 1008.64.

### Preparation of Intermediate Compound 127

To a solution of Intermediate Compound 126 (33 mg, 0.03 mmol) in dichloromethane (1.6 mL) was added trifluoroacetic acid (0.4 mL) at −78° C. under nitrogen. After stirred at room temperature for 1 hour, the reaction solution concentrated under reduced pressure. The resulting residue was purified by HPLC to afford Intermediate compound 127 (11 mg, 27%).

¹H-NMR (400 MHz, DMSO-d₆) δ 12.84 (s, 1H), 8.25 (s, 1H), 7.98 (s, 1H), 7.93 (s, 1H), 7.68-7.60 (m, 3H), 7.45 (s, 1H), 7.37-7.28 (m, 3H), 7.06 (d, J=9.2 Hz, 1H), 6.52 (s, 2H), 6.02 (dt, J=14.1, 6.5 Hz, 1H), 5.79 (s, 2H), 4.90 (dd, J=20.3, 7.7 Hz, 5H), 4.52 (dq, J=14.1, 6.3 Hz, 5H), 3.69 (s, 3H), 2.10 (d, J=10.6 Hz, 6H), 1.25 (q, J=7.9 Hz, 6H). EI-MS m/z: [M+H]⁺ 908.54.

### Example 19: Preparation of Intermediate Compound 133

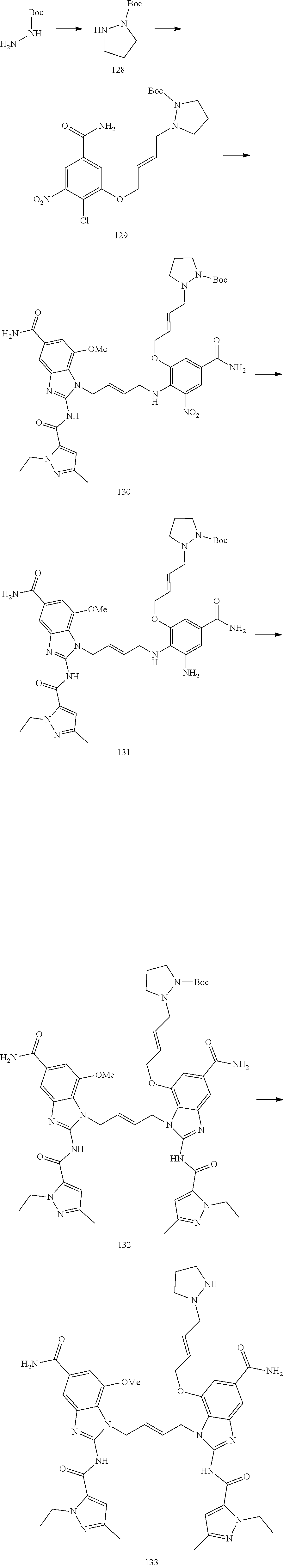

### Preparation of Intermediate Compound 128

To a solution of t-butyl carbazate (5 g, 37.83 mmol) in N,N-dimethylformamide (30 mL) was added sodium hydride (60%, 3.8 g, 94.6 mmol) at 0° C. After stirred at 0° C. for 0.5 hours, the reaction solution was added 1,3-dibromopropane (3.8 mL, 37.8 mmol) at room temperature for 3 hours. The reaction mixture was diluted with ethyl acetate (300 mL) and washed with distilled water (150 mL×2). The organic layer was dried over anhydrous sodium sulfate and then filtered. The filtrate was concentrated. The resulting residue was purified by column chromatography to afford Intermediate Compound 128 (3.9 g, 57%).

¹H-NMR (400 MHz, CDCl₃) δ 3.86 (s, 1H), 3.49-3.41 (m, 2H), 3.07-2.99 (m, 2H), 2.03 (p, J=6.8 Hz, 2H), 1.52-1.44 (m, 9H).

### Preparation of Intermediate Compound 129

To a solution of Intermediate Compound 128 (1.2 g, 6.86 mmol) and Intermediate Compound 5 (2.0 g, 5.72 mmol) in N,N-dimethylformamide (15 mL) was added potassium carbonate (1.1 g, 8.58 mmol) at room temperature under nitrogen. After stirred for 18 hours, the reaction mixture was diluted with ethyl acetate (200 mL) and washed with distilled water (100 mL×2). The organic layer was dried over anhydrous sodium sulfate and then filtered. The filtrate was concentrated. The resulting residue was purified by column chromatography to afford Intermediate Compound 129 (2.0 g, 80%).

¹H-NMR (400 MHz, DMSO) δ 8.28 (s, 1H), 8.05 (d, J=2.1 Hz, 1H), 7.88 (s, 1H), 7.78 (s, 1H), 6.02-5.83 (m, 2H), 4.81 (d, J=5.1 Hz, 2H), 3.26 (d, J=6.0 Hz, 2H), 2.84 (t, J=6.8 Hz, 2H), 2.00 (t, J=7.3 Hz, 2H), 1.37 (s, 9H).

### Preparation of Intermediate Compound 130

To a solution of Intermediate Compound 129 (2 g, 4.54 mmol) and Intermediate Compound 9 (4.7 g, 9.07 mmol) in n-butanol (35 mL) were added N,N-diisopropylethylamine (5.5 mL, 31.8 mmol) at room temperature and heated to 100° C. After stirred for 21 hours, the reaction mixture was cooled to room temperature and diluted with dichloromethane and diethyl ether. The resulting solid was filtered and washed ether. The filtered solid was purified by column chromatography to afford Intermediate Compound 130 (1.0 g, 28%). EI-MS m/z: [M+H]⁺ 816.52.

### Preparation of Intermediate Compound 131

To a solution of Intermediate Compound 130 (1.0 g, 1.29 mmol) in methanol (20 mL) and water (4 mL) were added ammonia solution (28-30% ammonia, 1.4 mL) and sodium hydrosulfite (Na₂S₂O₄, 2.2 g, 12.87 mmol) under nitrogen. After stirred at room temperature for 2 hours, the reaction solution was added methanol (50 mL). The resulting solid was filtered. The filtrate was concentrated and diluted with dichloromethane (100 mL) and washed with distilled water (50 mL). The organic layer was dried over anhydrous sodium sulfate and then filtered. The filtrate was concentrated. The resulting residue was purified by column chromatography to afford Intermediate Compound 131 (1.0 g, crude). EI-MS m/z: [M+H]⁺ 786.58.

### Preparation of Intermediate Compound 132

To a solution of Intermediate Compound 131 (1.0 g, 1.29 mmol) in N,N-dimethylformamide (6 mL) was added compound 2 (301 mg, 1.54 mmol) in N,N-dimethylformamide (3 mL) under nitrogen at room temperature. After stirred for 1 hour, the reaction mixture was added N-(3-dimethylaminopropyl)-N′-ethylcarbodiimide hydrochloride (0.34 mL, 1.93 mmol) and triethylamine (0.36 mL, 2.57 mmol) at room temperature for 17 hours. The reaction mixture was concentrated under reduced pressure. The resulting residue was purified by column chromatography to afford Intermediate Compound 132 (545 mg, 44%). EI-MS m/z: [M+H]⁺ 947.62.

### Preparation of Intermediate Compound 133

To a solution of Intermediate Compound 132 (63 mg, 0.07 mmol) was dissolved in dichloromethane (2 mL) and trifluoroacetic acid (0.2 mL) was added at −0° C. under nitrogen. After stirred at room temperature for 2 hours, the reaction mixture was concentrated under reduced pressure. The resulting residue was purified by HPLC to afford Intermediate Compound 133 (42 mg, 53%). EI-MS m/z: [M+H]⁺ 847.55.

### Example 20: Preparation of Intermediate Compound 139

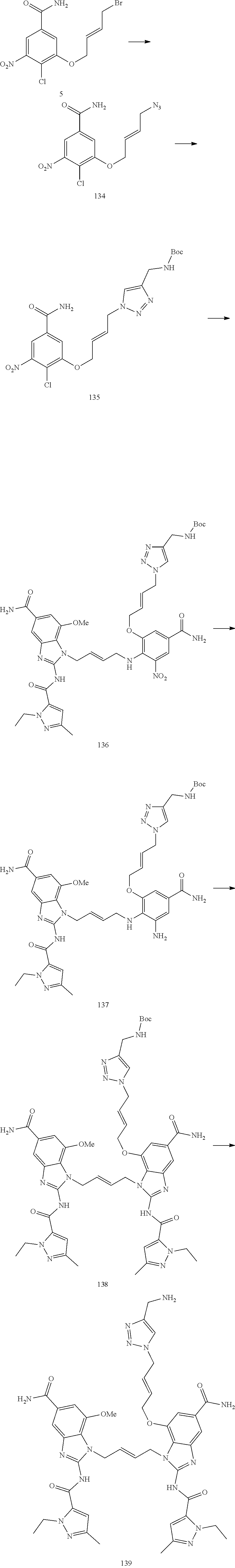

### Preparation of Intermediate Compound 134

To a solution of Intermediate Compound 5 (300 mg, 0.86 mmol) in N,N-dimethylformamide (5 mL) was added sodium azide (84 mg, 1.29 mmol) at 0° C. After stirred at 0° C. for 2 hours, the reaction mixture was diluted with ethyl acetate (100 mL) and washed with distilled water (150 mL×2). The organic layer was dried over anhydrous sodium sulfate and then filtered. The filtrate was concentrated and diluted with dichloromethane and hexane. The resulting solid was filtered and dried to afford Intermediate Compound 134 (225 mg, 84%), which was used without further purification.

¹H-NMR (400 MHz, DMSO) δ 8.27 (s, 1H), 8.07 (d, J=1.7 Hz, 1H), 7.90 (s, 1H), 7.78 (s, 1H), 6.11-5.94 (m, 2H), 4.88 (d, J=4.7 Hz, 2H), 3.97 (d, J=5.5 Hz, 2H).

### Preparation of Intermediate Compound 135

To a solution of Intermediate Compound 134 (225 mg, 0.72 mmol) in ethanol (3 mL), dichloromethane (2 mL) and water (3 mL) were added t-butyl propa-2-ynylcarbamate (145 mg, 0.94 mmol), copper(II) sulfate pentahydrate (36 mg, 0.14 mmol) and sodium L-ascorbate (57 mg, 0.29 mmol) at 0° C. After stirred at room temperature for 2 hours, the reaction mixture was diluted with dichloromethane (100 mL) and methanol (10 mL) and washed with saturated aqueous ammonium chloride solution (50 mL). The organic layer was dried over anhydrous sodium sulfate and then filtered. The filtrate was concentrated then diluted with dichloromethane and hexane. The resulting solid was filtered and dried to afford Intermediate Compound 135 (283 mg, 84%), which was used without further purification.

¹H-NMR (400 MHz, DMSO) δ 8.27 (s, 1H), 8.06 (s, 1H), 7.86 (d, J=8.3 Hz, 2H), 7.78 (s, 1H), 7.30 (s, 1H), 6.17-6.07 (m, 1H), 5.97-5.93 (m, 1H), 5.07 (d, J=6.0 Hz, 2H), 4.84 (d, J=5.2 Hz, 2H), 4.16 (d, J=5.9 Hz, 2H), 1.38 (s, 9H).

### Preparation of Intermediate Compound 136

To a solution of Intermediate Compound 135 (243 mg, 0.52 mmol) and Intermediate Compound 9 (428 mg, 1.04 mmol) in n-butanol (3 mL) were added N,N-diisopropylethylamine (0.45 mL, 2.60 mmol) at room temperature. After heated to 100° C. and stirred for 21 hours, the reaction solution was to room temperature. The reaction solution was diluted with dichloromethane (100 mL), methanol (20 mL) and washed with saturated aqueous ammonium chloride solution (50 mL). The organic layer was dried over anhydrous sodium sulfate and then filtered. The filtrate was concentrated. The resulting residue was purified by column chromatography to afford Intermediate Compound 136 (241 mg, 55%). EI-MS m/z: [M+H]⁺ 842.54.

### Preparation of Intermediate Compound 137

To a solution of Intermediate Compound 136 (241 mg, 0.29 mmol) in methanol (10 mL) and water (2 mL) were added ammonia solution (28-30% ammonia, 0.4 mL) and sodium hydrosulfite (Na₂S₂O₄, 498 mg, 2.86 mmol) to the reaction mixture under nitrogen. After stirred at room temperature for 2 hours, the reaction solution was added methanol (50 mL). The resulting solid was filtered. The filtrate was concentrated and diluted with dichloromethane (100 mL) and washed with distilled water (50 mL). The organic layer was dried over anhydrous sodium sulfate and then filtered. The filtrate was concentrated to afford Intermediate Compound 137 (168 mg, crude), which was used without further purification. EI-MS m/z: [M+H]⁺ 812.54.

### Preparation of Intermediate Compound 138

To a solution of Intermediate Compound 137 (168 mg, 0.21 mmol) in N,N-dimethylformamide (2 mL) was added compound 2 (48 mg, 0.25 mmol) in N,N-dimethylformamide (1 mL) to the reaction mixture under nitrogen at room temperature. After stirred for 1 hour, the reaction solution was added N-(3-dimethylaminopropyl)-N-ethylcarbodiimide hydrochloride (59 mg, 0.31 mmol) and triethylamine (0.09 mL, 0.62 mmol) at room temperature for 16 hours. The reaction mixture was concentrated under reduced pressure. The resulting residue was purified by column chromatography to afford Intermediate Compound 138 (175 mg, 87%). EI-MS m/z: [M+H]⁺ 973.60.

### Preparation of Intermediate Compound 139

To a solution of Intermediate Compound 138 (72 mg, 0.07 mmol) in dichloromethane (2 mL) was added trifluoroacetic acid (0.5 mL) at −0° C. under nitrogen. After stirred at room temperature for 2 hours, the reaction solution was concentrated under reduced pressure. The resulting residue was purified by HPLC to afford Intermediate Compound 139 (51 mg, 56%). EI-MS m/z: [M+H]⁺ 873.52.

### Example 21: Preparation of Intermediate Compound 147

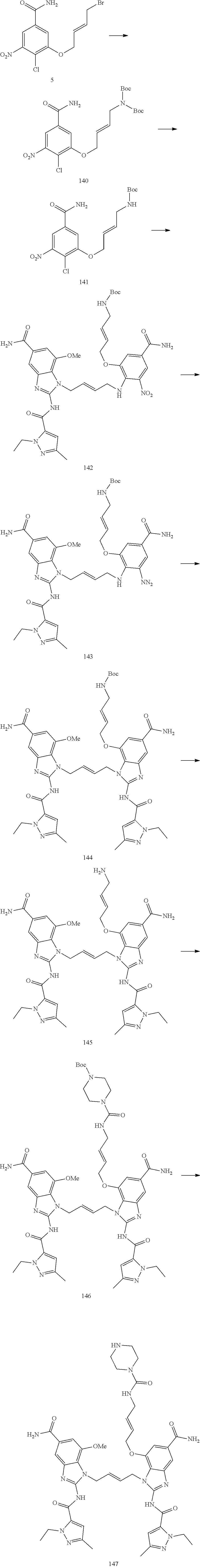

### Preparation of Intermediate Compound 140

To a solution of di-t-butyl-iminodi acetate (684 mg, 3.15 mmol) in N,N-dimethylformamide (7 mL) was added cesium carbonate (1.12 mg, 3.43 mmol). After stirred at room temperature for 10 minutes, the reaction solution was added Intermediate Compound 5 (1 g, 2.86 mmol) at room temperature for 17 hours. The reaction mixture was diluted with ethyl acetate (20 mL) and washed with distilled water (30 mL). The organic layer was dried over anhydrous magnesium sulfate and then filtered. The filtrate was concentrated under reduced pressure. The resulting residue was purified by column chromatography to afford Intermediate Compound 140 (1.05 g, 71%).

¹H-NMR (400 MHz, CDCl₃) δ 7.79 (d, J=1.9 Hz, 1H), 7.63 (d, J=1.9 Hz, 1H), 6.62 (s, 1H), 5.95 (dt, J=16.0, 5.2 Hz, 1H), 5.81 (dt, J=15.7, 5.6 Hz, 1H), 4.74 (d, J=5.4 Hz, 2H), 4.24 (d, J=5.1 Hz, 2H), 1.48 (s, 18H).

### Preparation of Intermediate Compound 141

To a solution of Intermediate Compound 140 (423 mg, 0.87 mmol) in methanol (4 mL) and tetrahydrofuran (10 mL) were added sodium hydroxide (105 mg, 2.61 mmol) in water (0.5 mL). After stirred at room temperature for 1 hour, the reaction mixture was diluted with ethyl acetate (20 mL) and washed with distilled water (30 mL). The organic layer was dried over anhydrous magnesium sulfate and then filtered. The filtrate was concentrated under reduced pressure. The resulting residue was purified by column chromatography to afford Intermediate Compound 141 (223 mg, 69%).

¹H-NMR (400 MHz, DMSO) δ 8.24 (s, 1H), 8.04 (d, J=2.2 Hz, 1H), 7.88 (s, 1H), 7.76 (s, 1H), 7.04 (s, 1H), 5.95-5.72 (m, 2H), 4.80 (d, J=5.5 Hz, 2H), 3.60 (d, J=6.0 Hz, 2H), 1.37 (s, 9H).

### Preparation of Intermediate Compound 142

To a solution of Intermediate Compound 141 (650 mg, 1.68 mmol) and compound 9 (831 mg, 2.02 mmol) in n-butanol (8 mL) was added N,N-diisopropylethylamine (1.2 mL, 8.42 mmol) at −0° C. After stirred for 5 minutes, the reaction mixture was heated to 120° C. for 20 hours. The reaction solution was concentrated under reduced pressure. The resulting residue was purified by column chromatography to afford Intermediate Compound 142 (297 mg, 23%). EI-MS m/z: [M+H]⁺ 761.45.

### Preparation of Intermediate Compound 143

To a solution of Intermediate Compound 142 (294 mg, 0.39 mmol) in methanol (6 mL) were added ammonia solution (28-30% ammonia, 0.7 mL, 9.5 mmol) and sodium hydrosulfite (Na₂S₂O₄, 672 mg, 3.86 mmol). After stirred at room temperature for 1 hours, the resulting solid was filtered through Celite and washed with methanol. The filtrate was concentrated under reduced pressure to afford Intermediate Compound 143 (202 mg, crude), which was used without further purification. EI-MS m/z: [M+H]⁺ 731.5.

### Preparation of Intermediate Compound 144

To a solution of Intermediate Compound 143 (202 mg, 0.28 mmol) in N,N-dimethylformamide (1 mL) was added compound 149 (30 mg, 0.15 mmol) in N,N-dimethylformamide (1 mL). After stirring at 0° C. for 30 minutes, the reaction solution was added N-(3-dimethylaminopropyl)-N-ethylcarbodiimide (43.7 mg, 0.28 mmol). The reaction solution was stirred at room temperature for 15 hours. The reaction mixture was concentrated under reduced pressure. The resulting residue was purified by column chromatography to afford Intermediate Compound 144 (122 mg, 49%). EI-MS m/z: [M+H]⁺ 892.5.

### Preparation of Intermediate Compound 145

To a solution of Intermediate Compound 144 (50 mg, 0.05 mmol) in dichloromethane (0.8 mL) was added trifluoroacetic acid (0.2 mL) at −0° C. After reaction mixture was raised to room temperature and stirred under nitrogen for 1 hour, the reaction mixture was concentrated under reduced pressure to afford Intermediate Compound 145 (63 mg, crude), which was used without further purification. EI-MS m/z: [M+H]⁺ 792.53.

### Preparation of Intermediate Compound 146

To a solution of Intermediate Compound 145 (35.4 mg, 0.04 mmol) in N,N-dimethylformamide (1 mL) was added N,N-diisopropylethylamine (0.04 mL, 0.22 mmol), carbonyldiimidazole (22 mg, 0.13 mmol), and 1-(t-butoxycarbonyl)piperazine (25 mg, 0.13 mmol). After stirred at room temperature for 20 hours, the reaction solution concentrated under reduced pressure to afford Intermediate Compound 146 (45 mg, crude), which was used without further purification. EI-MS m/z: [M+H]⁺ 1004.59.

### Preparation of Intermediate Compound 147

To a solution of Intermediate Compound 146 (45 mg, 0.04 mmol) in dichloromethane (1 mL) was added trifluoroacetic acid (0.2 mL) at −0° C. After stirred at room temperature under nitrogen for 1 hour, the reaction mixture was concentrated. The resulting residue was purified by HPLC to afford Intermediate Compound 147 (5.4 mg, 9.5%).

### Example 22: Preparation of Intermediate Compound 151

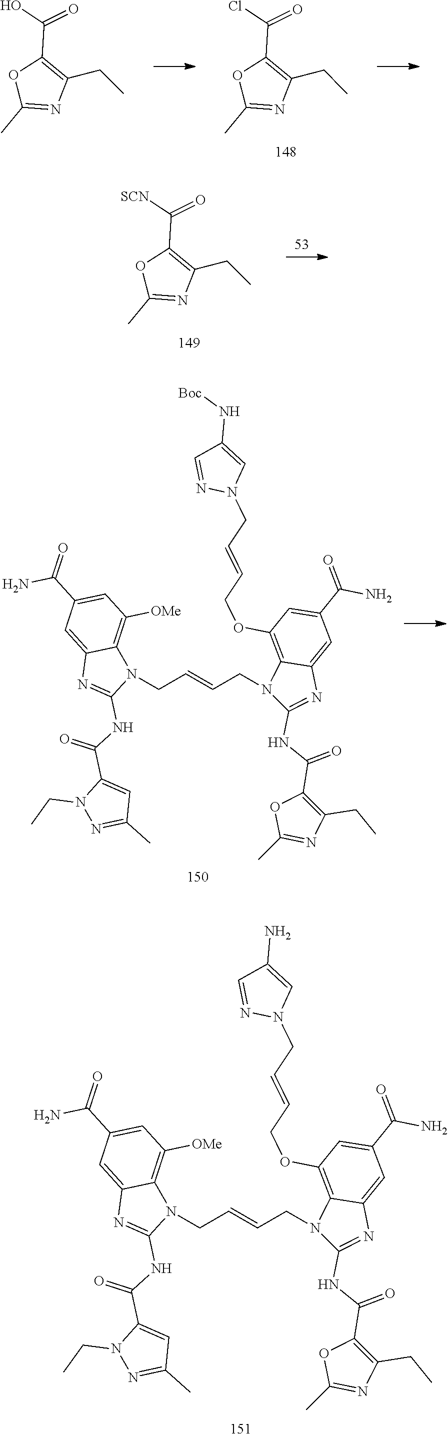

### Preparation of Intermediate Compound 148

To a solution of 4-ethyl-2-methyl-oxazole-5-carboxylic acid (100 mg, 0.64 mmol, prepared according to the method described in the Chinese patent publication No. CN 111471056 A) in tetrahydrofuran (1 mL) was added oxalyl chloride (0.82 mL, 0.96 mmol) and N,N-dimethylformamide (0.1 mL) at 0° C. After stirred at room temperature for 2 hours, the reaction mixture was concentrated under reduced pressure to afford Intermediate Compound 148 (crude), which was used without further purification.

### Preparation of Intermediate Compound 149

To a solution of Intermediate Compound 148 (crude) in acetone (1 mL) was added potassium thiocyanate (125 mg, 1.28 mmol) at 0° C. After stirred at room temperature for 30 minutes, the reaction solution was added hexane (10 mL). The resulting solid was filtered. The filtrate was concentrated under reduced pressure. The resulting residue was purified by column chromatography to afford Intermediate Compound 149 (64 mg, 50%).

¹H-NMR (400 MHz, CDCl₃), δ 2.90 (q, J=7.6 Hz, 2H), 2.54 (s, 3H), 2.72 (t, J=7.6 Hz, 3H). EI-MS m/z: [M+H]⁺ 197.21.

### Preparation of Intermediate Compound 150

To a solution of Intermediate Compound 53 (112 mg, 0.14 mmol) in N,N-dimethylformamide (1.5 mL) was added compound 149 (30 mg, 0.15 mmol) in N,N-dimethylformamide (1 mL). After stirred at 0° C. for 30 min, the reaction solution was added N-(3-dimethylaminopropyl)-N-ethylcarbodiimide (43.7 mg, 0.28 mmol) and triethylamine (0.06 mL, 0.42 mmol) and stirred at room temperature for 15 hours. The reaction mixture was concentrated under reduced pressure. The resulting residue was and purified by column chromatography to afford Intermediate Compound 150 (25 mg, 19%). EI-MS m/z: [M+H]⁺ 959.24.

### Preparation of Intermediate Compound 151

To a solution of Intermediate Compound 150 (25 mg, 0.03 mmol) in dichloromethane (0.8 mL) was added trifluoroacetic acid (0.2 mL) at −0° C. After stirred at room temperature for 2 hours. The reaction solution was concentrated. The resulting residue was purified by HPLC to afford Intermediate Compound 151 (9.3 mg, 42%).

¹H-NMR (400 MHz, DMSO-d₆) δ 8.00-7.83 (m, 2H), 7.65 (s, 1H), 7.54 (s, 1H), 7.36 (s, 1H), 7.31 (s, 1H), 6.51 (s, 1H), 5.91 (d, J=15.0 Hz, 1H), 5.76 (d, J=14.3 Hz, 2H), 4.89 (s, 3H), 4.66 (d, J=5.9 Hz, 1H), 4.51 (d, J=7.3 Hz, 2H), 3.71 (s, 2H), 2.82 (q, J=7.9 Hz, 1H), 2.40 (s, 2H), 2.10 (s, 2H), 1.25 (t, J=7.2 Hz, 2H), 1.02 (t, J=7.7 Hz, 2H). EI-MS m/z: [M+H]⁺ 859.27.

### Example 23: Preparation of Intermediate Compound 155

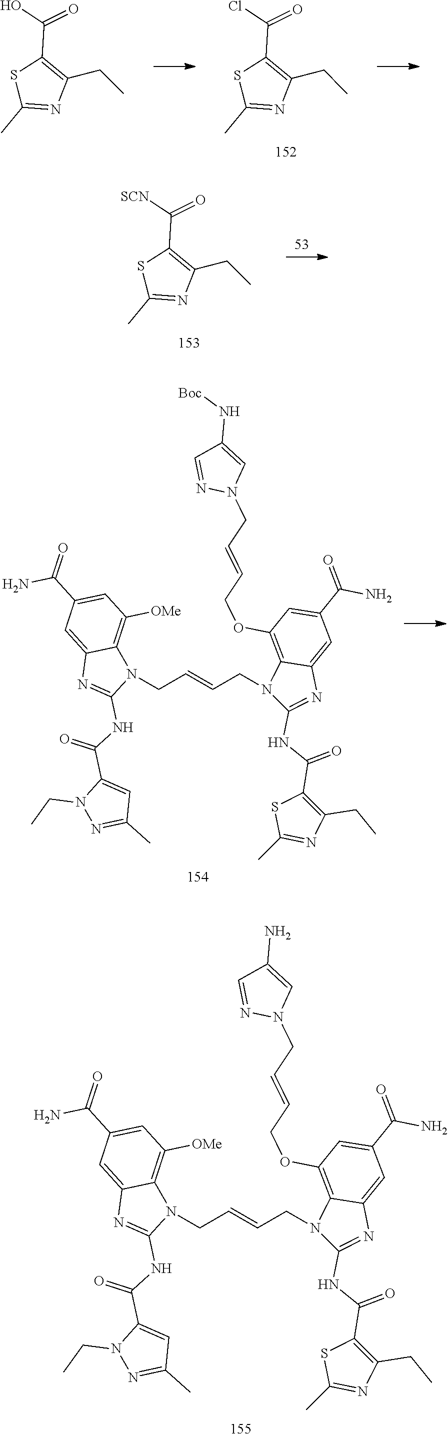

### Preparation of Intermediate Compound 152

To a solution of 4-ethyl-2-methylthiazole-5-carboxylic acid (100 mg, 0.58 mmol) in tetrahydrofuran (1 mL) were added oxalyl chloride (0.75 mL, 0.87 mmol) and N,N-dimethylformamide (0.1 mL) at 0° C. After stirred at room temperature for 2 hours, the reaction mixture was concentrated under reduced pressure to afford Intermediate Compound 152 (crude), which was used without further purification.

### Preparation of Intermediate Compound 153

To a solution of Intermediate Compound 152 (crude) in acetone (1 mL) was added potassium thiocyanate (113 mg, 1.16 mmol) at 0° C. After stirred at room temperature for 30 minutes, the reaction mixture was added hexane (10 mL). The resulting solid was filtered off. The filtrate was concentrated under reduced pressure. The resulting residue was purified by column chromatography to afford Intermediate Compound 153 (21 mg, 16%). EI-MS m/z: [M+H]⁺ 213.20.

### Preparation of Intermediate Compound 154

To a solution of Intermediate Compound 53 (166 mg, 0.15 mmol) in N,N-dimethylformamide (1.5 mL) was added Intermediate Compound 153 (35 mg, 0.16 mmol) dissolved in N,N-dimethylformamide (1 mL) at 0° C. After stirred for 30 minutes, the reaction solution was added N-(3-dimethylaminopropyl)-N-ethylcarbodiimide (46 mg, 0.29 mmol) and triethylamine (0.06 mL, 0.44 mmol) at room temperature for 15 hours. The reaction solution was concentrated under reduced pressure. The resulting residue was purified by column chromatography to afford Intermediate Compound 154 (30 mg, 21%). EI-MS m/z: [M+H]⁺ 975.26.

### Preparation of Intermediate Compound 155

To a solution of Intermediate Compound 154 (30 mg, 0.03 mmol) in dichloromethane (0.8 mL) was added trifluoroacetic acid (0.2 mL) at −0° C. After stirred at room temperature for 2 hours. The reaction solution was concentrated under reduced pressure. The resulting residue was purified by HPLC to afford Intermediate Compound 155 (18 mg, 68%).

¹H-NMR (400 MHz, DMSO-d₆) δ 7.88 (d, J=14.1 Hz, 1H), 7.82 (s, 1H), 7.56 (d, J=8.4 Hz, 1H), 7.47 (s, OH), 7.30-7.21 (m, 2H), 6.45 (s, 1H), 5.77 (dq, 2H), 4.81 (d, J=17.4 Hz, 2H), 4.60 (d, J=6.0 Hz, 1H), 4.51 (s, 1H), 4.44 (d, J=7.7 Hz, 1H), 3.64 (s, 2H), 3.03 (q, J=7.9 Hz, 2H), 2.66 (s, 2H), 2.02 (s, 2H), 1.19 (t, J=7.4 Hz, 2H), 1.07 (t, J=7.7 Hz, 2H). EI-MS m/z: [M+H]⁺ 875.24.

### Example 24: Preparation of Intermediate Compound 160

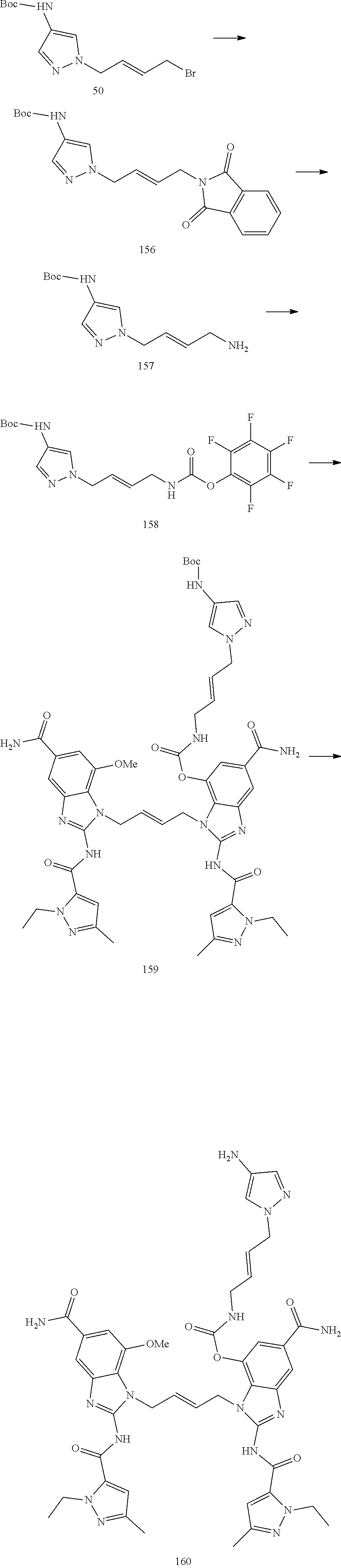 [SMILES: CC(C)(C)OC(=O)Nc1cnn(C/C=C/CBr)c1 ; CC(C)(C)OC(=O)Nc1cnn(C/C=C/CN2C(=O)c3ccccc3C2=O)c1 ; CC(C)(C)OC(=O)Nc1cnn(C/C=C/CN)c1 ; CC(C)(C)OC(=O)Nc1cnn(C/C=C/CNC(=O)Oc2c(F)c(F)c(F)c(F)c2F)c1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC(=O)NC/C=C/Cn3cc(NC(=O)OC(C)(C)C)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC(=O)NC/C=C/Cn3cc(N)cn3)c21]

### Preparation of Intermediate Compound 156

To a solution of methyl 4-nitro-1H-pyrazole-3-carboxylate (100 mg, 0.54 mmol) in N,N-dimethylformamide (3 mL) were added cesium carbonate (285 mg, 0.88 mmol) and trans-1,4-dibromo-2-butene (625 mg, 2.92 mmol). After stirred at room temperature for 2 hours, the reaction mixture was diluted with ethyl acetate (50 mL), washed with saturated aqueous ammonium chloride solution (50×2 mL). The organic layer was dried over anhydrous sodium sulfate and then filtered. The filtrate was concentrated under reduced pressure to afford Intermediate Compound 156 (592 mg, 94%), which was used without further purification.

### Preparation of Intermediate Compound 157

To a solution of Intermediate Compound 156 (592 mg, 1.55 mmol) in methanol (10 mL) was added hydrazine monohydrate (0.2 mL, 4.64 mmol). After stirred at room temperature for 1 hour, the reaction solution was added Dichloromethane and diethyl ether. The resulting solid was filtered. The filtrate was concentrated under reduced pressure. The resulting residue was purified by column chromatography to afford Intermediate Compound 157 (352 mg, 90%).

### Preparation of Intermediate Compound 158

To a solution of Intermediate Compound 157 (350 mg, 1.39 mmol) in dichloromethane (20 mL) were added N,N′-diisopropylethylamine (1.2 mL, 6.93 mmol) and bis(pentafluorophenyl)carbonate (1.6 g, 4.16 mmol) at −0° C. After stirred at room temperature for 1 hour, the reaction mixture was concentrated under reduced pressure. The reaction mixture was added dichloromethane and hexane. The resulting solid was filtered and dried to afford Intermediate Compound 158 (310 mg, 24%), which was used without further purification.

### Preparation of Intermediate Compound 159

To a solution of Intermediate Compound 158 (97 mg, 0.11 mmol) in N,N-dimethylformamide (2 mL) were added Intermediate Compound 75 (50 mg, 0.05 mmol) and N,N-diisopropylethylamine (0.05 mL, 0.26 mmol). After stirred at room temperature for 2 hours, the reaction mixture was concentrated under reduced pressure. The reaction mixture was added dichloromethane and hexane. The resulting solid was filtered and dried to afford Intermediate Compound 159 (59 mg, crude), which was used without further purification.

### Preparation of Intermediate Compound 160

To a solution of Intermediate Compound 159 (59 mg, crude) in dichloromethane (1.5 mL) was added trifluoroacetic acid (0.5 mL) at −0° C. After stirred at room temperature for 2 hours. The reaction mixture was concentrated under reduced pressure. The resulting residue was purified by HPLC to afford Intermediate Compound 160 (30 mg, 45%).

### Example 25: Preparation of Intermediate Compound 164

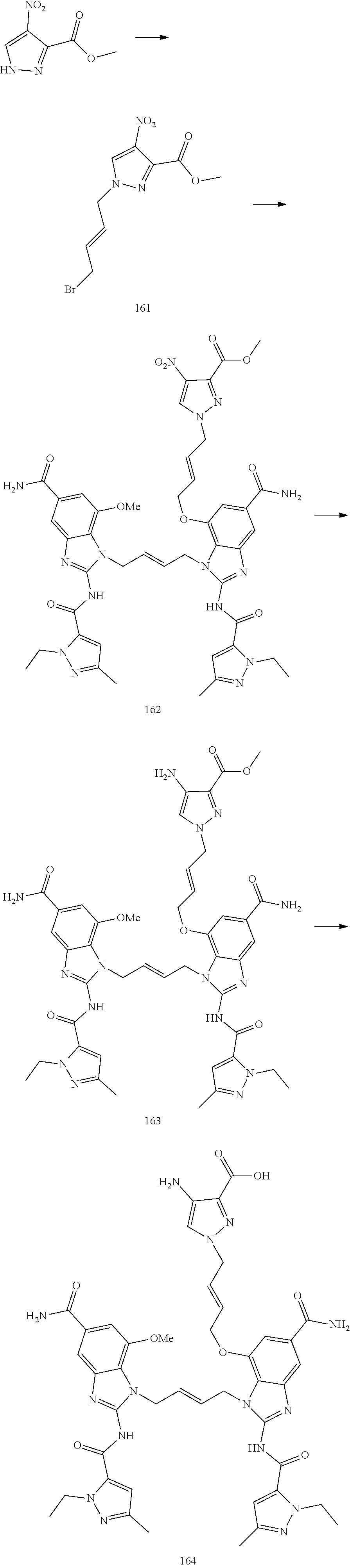 [SMILES: COC(=O)c1n[nH]cc1[N+](=O)[O-] ; COC(=O)c1nn(C/C=C/CBr)cc1[N+](=O)[O-] ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc([N+](=O)[O-])c(C(=O)OC)n3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(N)c(C(=O)OC)n3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(N)c(C(=O)O)n3)c21]

### Preparation of Intermediate Compound 161

To a solution of methyl 4-nitro-1H-pyrazole-3-carboxylate (100 mg, 0.54 mmol) in N,N-dimethylformamide (3 mL) were added cesium carbonate (285 mg, 0.88 mmol) and trans-1,4-dibromo-2-butene (625 mg, 2.92 mmol) After stirred at room temperature for 2 hours, the reaction mixture was diluted with ethyl acetate (50 mL) and washed with saturated aqueous ammonium chloride solution (50×2 mL). The organic layer was dried over anhydrous sodium sulfate and filtered. The filtrate was concentrated under reduced pressure. The resulting residue was purified by column chromatography to afford Intermediate Compound 161 (111 mg, 62%). EI-MS m/z: [M+H]⁺ 304.11.

### Preparation of Intermediate Compound 162

To a solution of Intermediate Compound 66 (50 mg, 0.07 mmol) in N,N-dimethylformamide (2 mL) were added cesium carbonate (34 mg, 0.1 mmol) and Intermediate Compound 161 (23 mg, 0.08 mmol). After stirred at room temperature for 2 hours, the reaction mixture was diluted with dichloromethane (20 mL) and washed with distilled water (30 mL). The organic layer was dried over anhydrous sodium sulfate and filtered. The filtrate was concentrated under reduced pressure. The resulting residue was purified by column chromatography to afford Intermediate Compound 162 (16 mg, 24%). EI-MS m/z: [M+H]⁺ 946.27.

### Preparation of Intermediate Compound 163

To a solution of Intermediate Compound 162 (67 mg, 0.07 mmol) in acetic acid (1 mL) was added zinc powder (46 mg). After stirred at room temperature for 2 hours, the reaction mixture was filtered through Celite then washed with methanol. The reaction mixture was concentrated under reduced pressure. The resulting residue was purified by HPLC to afford Intermediate Compound 163 (3.2 mg, 3.6%). EI-MS m/z: [M+H]⁺ 916.03.

### Preparation of Intermediate Compound 164

To a solution of Intermediate Compound 163 (43 mg, 0.05 mmol) in methanol (1.5 mL) was added lithium hydroxide monohydrate (24 mg, 0.14 mmol) dissolved in water (0.5 mL) at −50° C. After stirred at 0° C. for 20 hours, the reaction mixture was adjusted to pH 4-5 with acetic acid. The reaction mixture was concentrated under reduced pressure. The resulting residue was purified by HPLC to afford Intermediate Compound 164. (1 mg, 1.7%). EI-MS m/z: [M+H]⁺ 902.00.

### Example 26: Preparation of Intermediate Compound 166

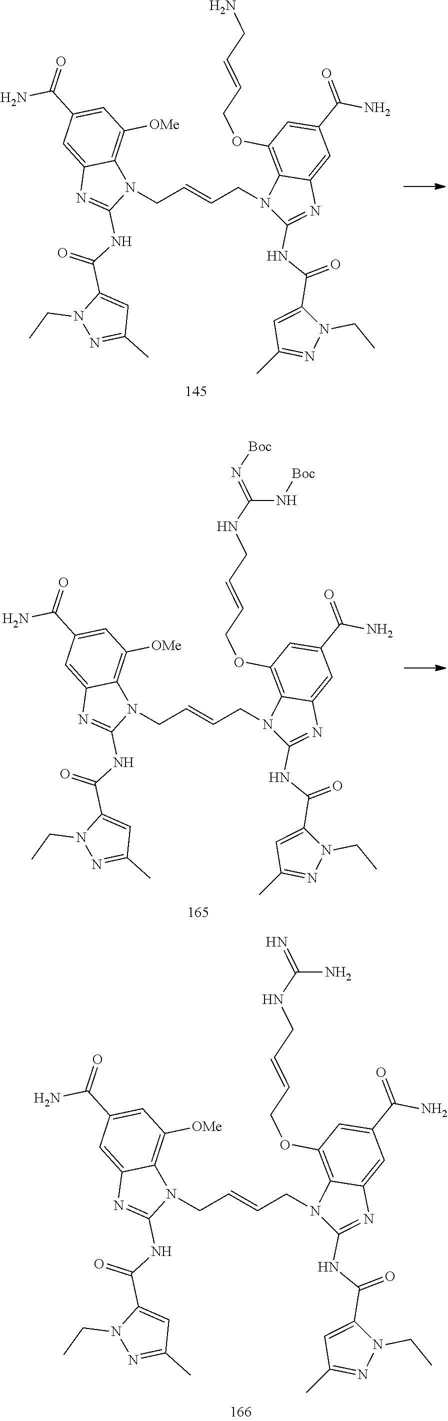 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN/C(=N/C(=O)OC(C)(C)C)NC(=O)OC(C)(C)C)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CNC(=N)N)c21]

### Preparation of Intermediate Compound 165

To a solution of Intermediate Compound 145 (40 mg, 0.04 mmol) in N,N-dimethylformamide (1 mL) were added triethylamine (0.10 mL, 0.745 mmol) and N,N-bis(t-butoxycarbonyl)-1H-pyrazole-1-carboxamidine (17 mg, 0.06 mmol) After stirred at room temperature for 3 hours, the reaction mixture was concentrated under reduced pressure to afford Intermediate Compound 165 (37 mg, crude), which was used without further purification. EI-MS m/z: [M+H]⁺ 1035.01.

### Preparation of Intermediate Compound 166

To a solution of Intermediate Compound 165 (45 mg, 0.04 mmol) in dichloromethane (2 mL) was added trifluoroacetic acid (0.5 mL) at −0° C. After stirred at room temperature for 1 hour, the reaction mixture was concentrated. The resulting residue was purified by HPLC to afford Intermediate Compound 166 (23.8 mg, 46%). EI-MS m/z: [M+H]⁺ 835.09.

### Example 27: Preparation of Intermediate Compound 168

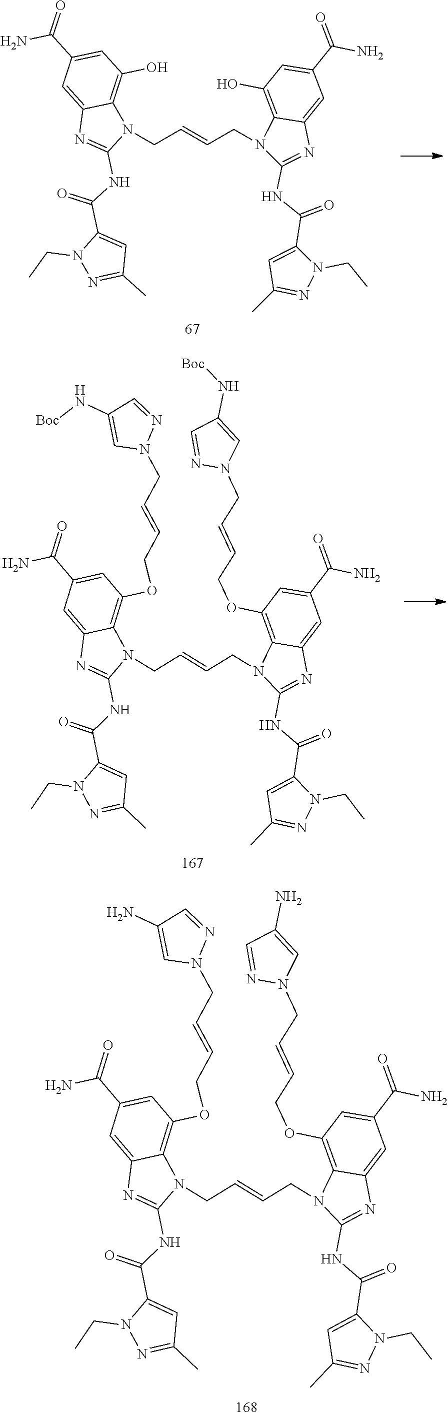 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(O)c21 ; CCn1nc(C)cc1C(=O)Nc1nc2cc(C(N)=O)cc(OC/C=C/Cn3cc(NC(=O)OC(C)(C)C)cn3)c2n1C/C=C/Cn1c(NC(=O)c2cc(C)nn2CC)nc2cc(C(N)=O)cc(OC/C=C/Cn3cc(NC(=O)OC(C)(C)C)cn3)c21 ; CCn1nc(C)cc1C(=O)Nc1nc2cc(C(N)=O)cc(OC/C=C/Cn3cc(N)cn3)c2n1C/C=C/Cn1c(NC(=O)c2cc(C)nn2CC)nc2cc(C(N)=O)cc(OC/C=C/Cn3cc(N)cn3)c21]

### Preparation of Intermediate Compound 167

To a solution of Intermediate Compound 67 (2.0 g, 2.07 mmol, Intermediate Compound 67 was prepared according to the method described in the International patent publication No. WO 2022/155518 A1) in N,N-dimethylformamide (20 mL) were added cesium carbonate (5.4 g, 16.54 mmol) and Intermediate Compound 50 (654 mg, 2.07 mmol) in N,N-dimethylformamide (5 mL). After stirred at room temperature for 3 hours, the reaction mixture was concentrated under reduced pressure and chloroform (100 mL) and methanol (20 mL) were added and washed with distilled water (30 mL). The organic layer was dried over anhydrous sodium sulfate and filtered. The filtrate was concentrated under reduced pressure. The resulting residue was purified by reversed-phase column chromatography to afford Intermediate Compound 167 (958 mg, 38%). EI-MS m/z: [M+H]⁺ 1179.46.

### Preparation of Intermediate Compound 168

To a solution of Intermediate Compound 167 (40 mg, 0.03 mmol) in dichloromethane (1 mL) was added trifluoroacetic acid (0.2 mL) at −0° C. After stirred at room temperature under nitrogen for 1.5 hour, the reaction mixture was concentrated. The resulting residue was purified by HPLC to afford Intermediate Compound 168 (17 mg, 29%). EI-MS m/z: [M+H]⁺ 979.29.

### Example 28: Preparation of Intermediate Compound 175

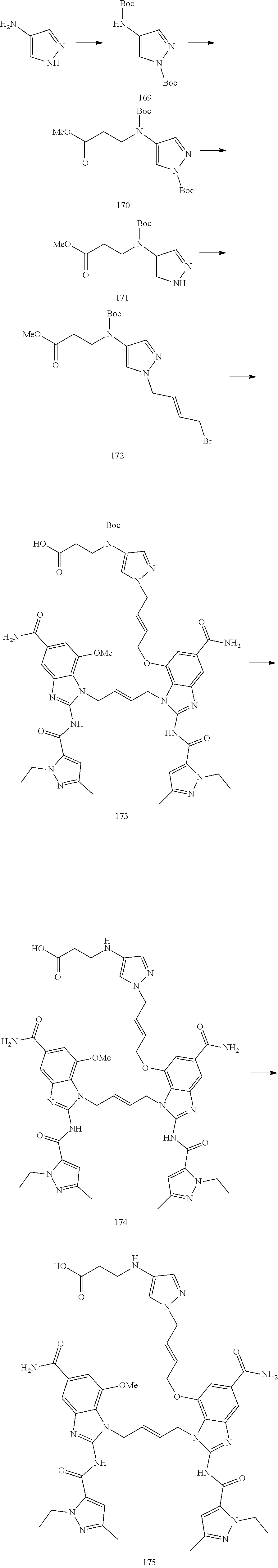

### Preparation of Intermediate Compound 169

To a solution of 4-aminopyrazole (5.89 g, 70.8 mmol) was in tetrahydrofuran (200 mL) were added triethylamine (15 mL, 106.13 mmol) and di-t-butyl dicarbonate (48.8 mL, 212.26 mmol) under nitrogen. After stirred at room temperature for 20 hours, the reaction mixture was added ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 169 (5.9 g, 29%).

¹H-NMR (400 MHz, CDCl3) δ 8.19 (s, 1H), 7.63 (s, 1H), 6.34 (s, 1H), 1.64 (d, J=3.9 Hz, 9H), 1.52 (s, 9H).

### Preparation of Intermediate Compound 170

To a solution of Intermediate Compound 169 (1.1 g, 3.88 mmol) in acetonitrile (30 mL) were added potassium carbonate (590 mg, 4.27 mmol), 18-crown-6 (513 mg, 1.94 mmol) and methyl acrylate (367 mg, 4.27). After stirred at room temperature for 30 minutes, the reaction solution was added Ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 170. (1.38 g, 96%).

¹H-NMR (400 MHz, CDCl3) δ 7.77 (s, 1H), 7.27 (d, J=3.1 Hz, 1H), 3.92 (dt, J=9.4, 5.8 Hz, 2H), 3.71-3.65 (m, 3H), 2.68-2.59 (m, 2H), 1.65 (q, J=2.4 Hz, 9H), 1.54-1.48 (m, 9H). EI-MS m/z: [M+H]⁺ 370.32.

### Preparation of Intermediate Compound 171

To a solution of Intermediate Compound 170 (1.38 g, 3.73 mmol) in methanol (20 mL) was added potassium carbonate (770 mg, 5.6 mmol) at −0° C. After stirred at room temperature for 30 minutes, the reaction solution was added Ethyl acetate (50 mL) was added and washed with distilled water (50 mL×2). The organic layer was dried over anhydrous sodium sulfate and filtered. The filtrate was concentrated under reduced pressure. The resulting residue was purified by column chromatography to afford Intermediate Compound 171. (950 mg, 94%).

¹H-NMR (400 MHz, CDCl3) δ 7.66 (s, 1H), 7.53 (s, 1H), 3.96-3.87 (m, 2H), 3.67 (s, 3H), 2.64 (p, J=5.0 Hz, 2H), 1.50 (s, 9H).

### Preparation of Intermediate Compound 172

To a solution of Intermediate Compound 171 (167 mg, 0.62 mmol) in N,N-dimethylformamide (30 mL) were added cesium carbonate (303 mg, 0.93 mmol) and trans-1,4-dibromo-2-butene (397 mg, 1.86 mmol). After stirred at room temperature for 3 hours, the reaction solution was Ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 172 (177 mg, 71%). EI-MS m/z: [M+H]⁺ 402.24.

### Preparation of Intermediate Compound 173

To a solution of Intermediate Compound 66 (264 mg, 0.37 mmol) in N,N-dimethylformamide (2 mL) were added cesium carbonate (179 mg, 0.43 mmol) and compound 172 (177 mg, 0.44 mmol) After stirred at room temperature for 12 hours, the reaction solution was added Ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 173 (133 mg, 34%). EI-MS m/z: [M+H]⁺ 1045.34.

### Preparation of Intermediate Compound 174

To a solution of Intermediate Compound 173 (150 mg, 0.14 mmol) in dichloromethane (1 mL) was added trifluoroacetic acid (0.25 mL) at −0° C. After stirred at room temperature for 1 hour, the reaction mixture was concentrated under reduced pressure to afford Intermediate Compound 174 (135 mg, crude), which was used without further purification.

### Preparation of Intermediate Compound 175

To a solution of Intermediate Compound 174 (135 mg, crude) in methanol (1 mL) was added lithium hydroxide monohydrate (11.7 mg, 0.28 mmol) in water (1 mL) at −45° C. After stirred at −0° C. for 2 hours, the reaction mixture was adjusted to pH 4-5 with acetic acid. The reaction solution was concentrated under reduced pressure. The resulting residue was purified by HPLC to afford Intermediate Compound 175 (34 mg, 42%). EI-MS m/z: [M+H]⁺ 902.

### Example 29: Preparation of Intermediate Compound 178

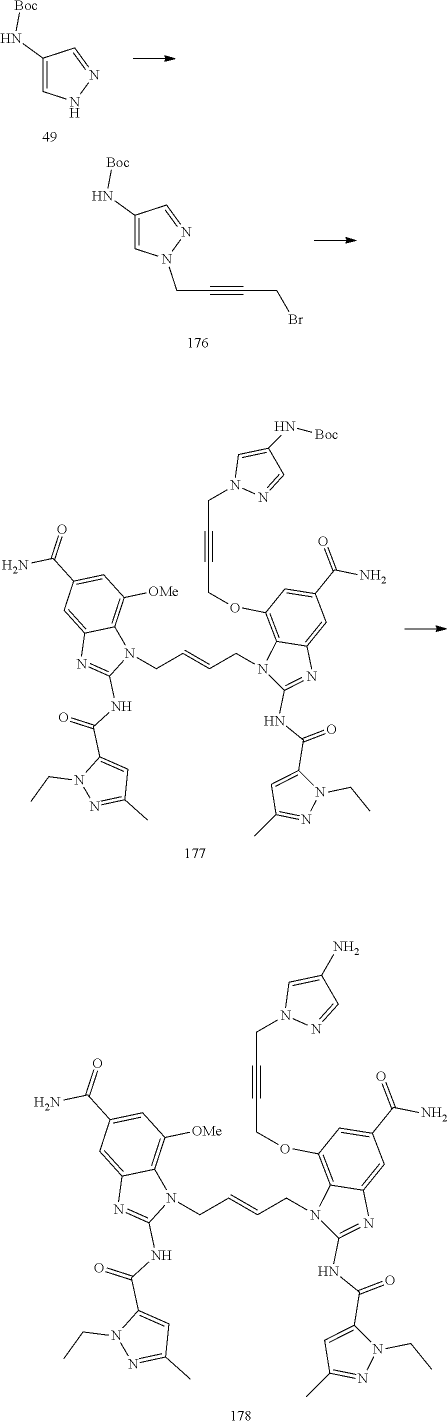 [SMILES: CC(C)(C)OC(=O)Nc1cn[nH]c1 ; CC(C)(C)OC(=O)Nc1cnn(CC#CCBr)c1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCC#CCn3cc(NC(=O)OC(C)(C)C)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCC#CCn3cc(N)cn3)c21]

### Preparation of Intermediate Compound 176

To a solution of Intermediate Compound 49 (300 mg, 1.64 mmol) in N,N-dimethylformamide (10 mL) were added cesium carbonate (693 mg, 2.13 mmol) and 1,4-dibromo-2-butene (1.04 g, 4.91 mmol). After stirred at room temperature for 1 hours, the reaction mixture was diluted with ethyl acetate (50 mL), washed with saturated aqueous ammonium chloride solution (2×50 mL). The organic layer was dried over anhydrous sodium sulfate and filtered. The filtrate was concentrated under reduced pressure. The resulting residue was purified by column chromatography to afford Intermediate Compound 176 (320 mg, 620%).

### Preparation of Intermediate Compound 177

To a solution of Intermediate Compound 66 (400 mg, 0.42 mmol, Intermediate Compound 66 was prepared according to the method described in the International patent publication No. WO 2022/155518 A1) in N,N-dimethylformamide (5 mL) were added cesium carbonate (548 mg, 1.68 mmol) and Intermediate Compound 176 (158 mg, 0.50 mmol) in N,N-dimethylformamide (2 mL). After stirred at room temperature for 3 hours, the reaction mixture was concentrated under reduced pressure. The reaction mixture was added dichloromethane (50 mL) and methanol (10 mL) and washed with distilled water (30 mL). The organic layer was dried over anhydrous sodium sulfate and filtered. The filtrate was concentrated under reduced pressure. The resulting residue was purified by column chromatography to afford Intermediate Compound 177 (246 mg, 61%). EI-MS m/z: [M+H]⁺ 956.52.

### Preparation of Intermediate Compound 178

To a solution of Intermediate Compound 177 (246 mg, 0.26 mmol) in dichloromethane (3 mL) was added trifluoroacetic acid (1 mL) at −0° C. After stirred at room temperature under nitrogen for 1.5 hour, the reaction mixture was concentrated under reduced pressure. The resulting residue was purified by HPLC to afford Intermediate Compound 178 (92 mg, 30%). EI-MS m/z: [M+H]⁺ 856.48.

### Example 30: Preparation of Intermediate Compound 187

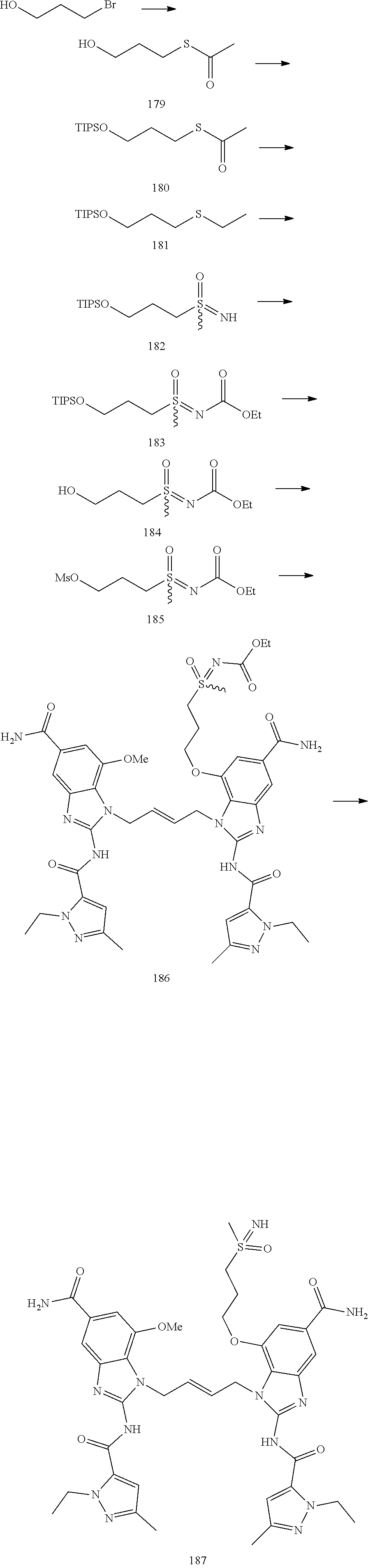

### Preparation of Intermediate Compound 179

To a solution of 3-bromopropanol (2.0 g, 14.39 mmol) in acetone (30 mL) was added potassium thiocyanate (1.8 g, 15.83 mmol) at −0° C. under nitrogen. After stirred at room temperature for 17 hours, the reaction mixture was concentrated under reduced pressure. The resulting residue was purified by column chromatography to afford Intermediate Compound 179 (1.26 g, 65%).

¹H-NMR (400 MHz, CDCl₃) δ 3.65 (q, J=5.6 Hz, 2H), 3.01 (dq, J=9.3, 3.3 Hz, 2H), 2.39-2.30 (m, 3H), 2.04 (t, J=5.8 Hz, 1H), 1.83 (q, J=6.0 Hz, 2H).

### Preparation of Intermediate Compound 180

To a solution of Intermediate Compound 179 (1.26 g, 9.39 mmol) in dichloromethane (30 mL) were added imidazole (958 mg, 14.08 mmol) and triisopropylsilyl chloride (2.0 g, 10.33 mmol) at −0° C. After stirred at room temperature for 4 hours, the reaction mixture was diluted with dichloromethane (100 mL), washed with saturated aqueous ammonium chloride solution (70 mL) and distilled water (70 mL). The organic layer was dried over anhydrous sodium sulfate and filtered. The filtrate was concentrated under reduced pressure. The resulting residue was purified by column chromatography to afford Intermediate Compound 180 (2.7 g, 99%).

¹H-NMR (400 MHz, CDCl₃) δ 3.74 (q, J=5.4 Hz, 2H), 2.99 (q, J=6.3 Hz, 2H), 2.35-2.30 (m, 3H), 1.81 (q, J=6.2 Hz, 2H), 1.06 (s, 21H).

### Preparation of Intermediate Compound 181

To a solution of Intermediate Compound 180 (2.7 g, 9.29 mmol) in methanol (30 mL) were added methyl iodide (0.69 mL, 10.2 mmol) and potassium carbonate (4.1 g, 30.25 mmol) at −0° C. After stirred at room temperature for 30 minutes, the reaction mixture was diluted with dichloromethane (100 mL) and washed with distilled water (70 mL). The organic layer was dried over anhydrous sodium sulfate and filtered. The filtrate was concentrated under reduced pressure. The resulting residue was purified by column chromatography to afford Intermediate Compound 181 (1.73 g, 65%).

¹H-NMR (400 MHz, CDCl₃) δ 3.86-3.73 (m, 2H), 2.60 (tt, J=7.0, 2.5 Hz, 2H), 2.11 (q, J=2.3 Hz, 3H), 1.82 (q, J=6.2 Hz, 2H), 1.15-1.02 (m, 21H).

### Preparation of Intermediate Compound 182

To a solution of Intermediate Compound 181 (1.2 g, 4.57 mmol) in methanol (20 mL) were added iodobenzene diacetate (3.7 g, 11.43 mmol) and ammonium carbonate (1.3 g, 13.71 mmol) at −0° C. After stirred and reflux for 2 hours, the reaction mixture was concentrated. The resulting residue was purified by purified by HPLC to afford Compound 182 (1.5 g, crude).

¹H-NMR (400 MHz, CDCl₃) δ 3.84 (q, J=5.4 Hz, 2H), 3.28-3.21 (m, 2H), 3.03-2.97 (m, 3H), 2.11-2.03 (m, 2H), 1.06 (d, J=4.4 Hz, 21H).

### Preparation of Intermediate Compound 183

To a solution of Intermediate Compound 182 (1.5 g, crude) in dichloromethane (20 mL) were added pyridine (0.71 mL, 8.86 mmol) and ethyl chloroformate (0.51 mL, 5.31 mmol) at 0° C. After stirred at room temperature for 2 hours, the reaction mixture was diluted with dichloromethane (100 mL), washed with 0.5 N hydrochloric acid solution (70 mL) and distilled water (70 mL). The organic layer was dried over anhydrous sodium sulfate and filtered. The filtrate was concentrated under reduced pressure. The resulting residue was purified by column chromatography to afford Intermediate Compound 183 (1.47 g, 91%).

¹H-NMR (400 MHz, CDCl₃) δ 4.15 (s, 2H), 3.83 (t, J=5.2 Hz, 2H), 3.51 (d, J=10.0 Hz, 2H), 3.25 (s, 3H), 2.10 (s, 2H), 1.29 (t, J=5.8 Hz, 3H), 1.06 (d, J=4.3 Hz, 21H).

### Preparation of Intermediate Compound 184

To a solution of Intermediate Compound 183 (1.5 g, 4.02 mmol) in dichloromethane (30 mL) was added hydrochloric acid (4 M 1,4-dioxane solution, 12 mL) at 0° C. After stirred for 2.5 hours, the reaction mixture was concentrated. The reaction mixture was added ethyl acetate (100 mL) and distilled water (70 mL). The obtained aqueous layer was concentrated. The reaction mixture was added dichloromethane (50 mL), methanol (5 mL). The organic layer was dried over anhydrous sodium sulfate and filtered. The filtrate was concentrated under reduced pressure to afford Intermediate Compound 184 (752 mg, 89%).

¹H-NMR (400 MHz, CDCl₃) δ 4.14 (tt, J=8.5, 4.5 Hz, 2H), 3.81 (t, J=5.4 Hz, 2H), 3.67-3.42 (m, 2H), 3.31-3.25 (m, 3H), 2.16 (q, J=6.1 Hz, 2H), 1.29 (dt, J=8.7, 4.8 Hz, 3H).

### Preparation of Intermediate Compound 185

To a solution of Intermediate Compound 184 (50 mg, 0.24 mmol) in dichloromethane (3 mL) were added triethylamine (0.07 mL, 0.48 mmol) and methanesulfonyl anhydride (50 mg, 0.29 mmol) at −0° C. After stirred at room temperature for 2 hours, the reaction mixture was diluted with dichloromethane (50 mL) and washed with distilled water (20 mL). The organic layer was dried over anhydrous sodium sulfate and filtered. The filtrate was concentrated to afford Intermediate Compound 185 (50 mg, 73%), which was used without further purification.

¹H-NMR (400 MHz, CDCl₃) δ 4.41 (d, J=5.9 Hz, 2H), 4.15 (dq, J=10.5, 3.7 Hz, 2H), 3.51 (d, J=53.9 Hz, 2H), 3.30 (q, J=2.4 Hz, 3H), 3.06 (q, J=2.4 Hz, 3H), 2.40 (d, J=8.7 Hz, 2H), 1.35-1.25 (m, 3H).

### Preparation of Intermediate Compound 186

To a solution of Intermediate Compound 66 (100 mg, 0.11 mmol, Intermediate Compound 66 was prepared according to the method described in the International patent publication No. WO 2022/155518 A1) in N,N-dimethylformamide (2 mL) were added cesium carbonate (113 mg, 0.35 mmol) and Intermediate Compound 185 (36 mg, 0.13 mmol) dissolved in N,N-dimethylformamide (1 mL). After stirred at room temperature for 3 hours, the reaction mixture was concentrated under reduced pressure. The reaction mixture was added dichloromethane (50 mL) and methanol (10 mL) and washed with distilled water (30 mL). The organic layer was dried over anhydrous sodium sulfate and filtered. The filtrate was concentrated under reduced pressure. The resulting residue was purified by column chromatography to afford Intermediate Compound 186 (48 mg, 50%). EI-MS m/z. [M+H]⁺ 914.41.

### Preparation of Intermediate Compound 187

To a solution of Intermediate Compound 186 (48 mg, 0.05 mmol) in ethanol (20 mL) was added sodium ethoxide (21% w/w ethanol, 0.24 mL, 0.64 mmol). After stirred and reflux for 14 hours, the reaction mixture was concentrated. The resulting residue was purified by HPLC to afford Intermediate Compound 187 (25 mg, 55%). EI-MS m/z: [M+H]⁺ 842.39.

### Example 31: Preparation of Intermediate Compound 197

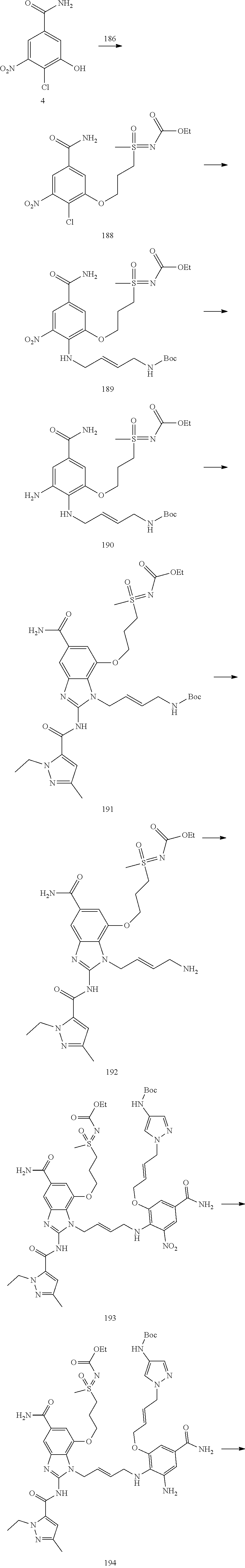

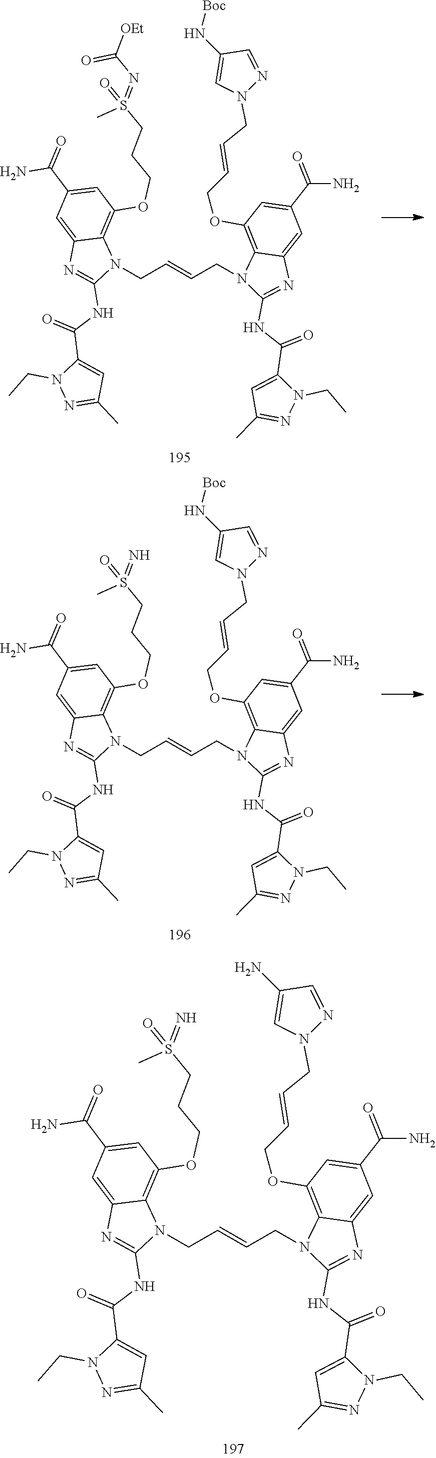

### Preparation of Intermediate Compound 188

To a solution of Intermediate Compound 4 (400 mg, 1.85 mmol) in N,N-dimethylformamide (5 mL) were added cesium carbonate (782 mg, 2.40 mmol) and compound 185 (584 mg, 2.03 mmol). After stirred at room temperature for 2 hours, the reaction mixture was diluted with ethyl acetate (50 mL) and washed with saturated aqueous ammonium chloride solution (50×2 mL). The organic layer was dried over anhydrous sodium sulfate and filtered. The filtrate was concentrated under reduced pressure. The resulting residue was purified by column chromatography to afford Intermediate Compound 188 (600 mg, 80%).

¹H-NMR (400 MHz, CDCl₃) δ 7.85 (s, 1H), 7.73 (d, J=4.3 Hz, 1H), 4.38 (q, J=6.0 Hz, 2H), 4.13 (q, J=6.5 Hz, 2H), 3.81-3.65 (m, 1H), 3.54 (d, J=14.8 Hz, 1H), 3.37-3.25 (m, 3H), 2.56-2.48 (m, 2H), 1.29 (q, J=6.6 Hz, 3H).

### Preparation of Intermediate Compound 189

To a solution of Intermediate Compound 188 (600 mg, 1.47 mmol) in ethanol (10 mL) were added t-butyl (E)-(4-aminobut-2-en-1-yl)carbamate (548 mg, 2.94 mmol) and triethylamine (0.62 mL, 4.41 mmol). After stirred at 120° C. for 20 hours, the reaction mixture was concentrated under reduced pressure. The resulting residue was purified by column chromatography to afford Intermediate Compound 189 (550 mg, 67%). EI-MS m/z: [M+H]⁺ 558.31.

### Preparation of Intermediate Compound 190

To a solution of Intermediate Compound 189 (550 mg, 0.99 mmol) in methanol (5 mL) and distilled water (1 mL) were added ammonia solution (28-30% ammonia, 1 mL) and sodium hydrosulfite (Na₂S₂O₄, 1.7 g, 9.86 mmol) at −0° C. After stirred at room temperature for 1.5 hour, the reaction solution was added methanol (10 mL). The resulting solid was filtered and washed with methanol. The filtrate was concentrated under reduced pressure. The reaction mixture was added and washed with distilled water (20 mL). The organic layer was dried over anhydrous sodium sulfate and filtered. The filtrate was concentrated under reduced pressure to afford Intermediate Compound 190 (339 mg, 65%), which was used without further purification. EI-MS m/z: [M+H]⁺ 528.39.

### Preparation of Intermediate Compound 191

To a solution of Intermediate Compound 190 (339 mg, 0.64 mmol) in N,N-dimethylformamide (3 mL) was added Intermediate Compound 2 (150 mg, 0.77 mmol) at −0° C. After stirred at room temperature for 30 minutes, the reaction solution was added N-(3-dimethylaminopropyl)-N′-ethylcarbodiimide hydrochloride (160 mg, 0.84 mmol) and triethylamine (0.05 mL, 0.38 mmol) at room temperature for 17 hours. The reaction mixture was concentrated under reduced pressure. The resulting residue was purified by column chromatography to afford Intermediate Compound 191 (390 mg, 88%). EI-MS m/z: [M+H]⁺ 689.37.

### Preparation of Intermediate Compound 192

To a solution of Intermediate Compound 191 (390 mg, 0.57 mmol) in dichloromethane (5 mL) and methanol (1 mL) was added hydrochloric acid (4 M 1,4-dioxane solution, 1.5 mL). After stirred for 2 hours, the reaction mixture was concentrated. The reaction mixture was added diethyl ether (20 mL). The resulting solid was filtered and dried to afford Intermediate Compound 192 (360 mg, 96%). EI-MS m/z: [M+H]⁺ 589.38.

### Preparation of Intermediate Compound 193

To a solution of Intermediate Compound 192 (360 mg, 0.54 mmol) in n-butanol (3 mL) were added compound 51 (170 mg, 0.38 mmol) and triethylamine (0.26 mL, 1.88 mmol) −0° C. After stirred at 120° C. for 24 hours, the reaction mixture cooled to room temperature. The reaction mixture was concentrated under reduced pressure. The resulting residue was purified by column chromatography to afford Intermediate Compound 193 (127 mg, 34%). EI-MS m/z: [M+H]⁺ 1004.40.

### Preparation of Intermediate Compound 194

To a solution of Intermediate Compound 193 (127 mg, 0.13 mmol) in methanol (5 mL) and distilled water (1 mL) were added ammonia solution (28˜30% ammonia, 0.2 mL) and sodium hydrosulfite (220 mg, 1.26 mmol) at −0° C. After stirred at room temperature for 2.5 hours, the reaction mixture was added methanol (10 mL). The resulting solid was filtered and washed with methanol. The filtrate was concentrated under reduced pressure. The reaction mixture was added dichloromethane (60 mL) and washed with distilled water (20 mL). The organic layer was dried with anhydrous sodium sulfate and filtered. The filtrate was concentrated to afford Intermediate Compound 194 (74 mg, 60%), which was used without further purification. EI-MS m/z: [M+H]⁺ 974.47.

### Preparation of Intermediate Compound 195

To a solution of Intermediate Compound 194 (74 mg, 0.08 mmol) in N,N-dimethylformamide (1 mL) was added compound 2 (18 mg, 0.09 mmol) at −0° C. After stirred at room temperature for 15 minutes, the reaction mixture was added N-(3-dimethylaminopropyl)-N′-ethylcarbodiimide hydrochloride (19 mg, 0.10 mmol) and triethylamine (0.05 mL, 0.38 mmol) at room temperature for 15 hours. The reaction mixture was concentrated under reduced pressure. The resulting residue was purified by column chromatography to afford Intermediate Compound 195 (42 mg, 49%). EI-MS m/z: [M+H]⁺ 1135.55.

### Preparation of Intermediate Compound 196

To a solution of Intermediate Compound 195 (42 mg, 0.04 mmol) in ethanol (2 mL) was added sodium ethoxide (21% w/w ethanol, 0.14 mL, 0.37 mmol). After stirred at reflux for 7 hours, the reaction mixture was concentrated under reduced pressure to afford Intermediate Compound 196 (50 mg, crude), which was used without further purification. EI-MS m/z: [M+H]⁺ 1063.53.

### Preparation of Intermediate Compound 197

To a solution of Intermediate Compound 196 (50 mg, crude) in dichloromethane (3 mL) was added trifluoroacetic acid (1 mL) at −0° C. under nitrogen. After stirred at room temperature for 1 hour, the reaction mixture was concentrated under reduced pressure. The resulting residue was purified by HPLC to afford Intermediate Compound 197 (23 mg, 49%).

¹H-NMR (400 MHz, DMSO) δ 7.96 (d, J=20.3 Hz, 2H), 7.86 (d, J=0.8 Hz, 1H), 7.67 (dd, J=14.8, 1.2 Hz, 2H), 7.41-7.36 (m, 2H), 7.28 (dd, J=7.1, 1.4 Hz, 2H), 6.53 (s, 2H), 5.87-5.54 (m, 5H), 4.95-4.85 (m, 4H), 4.54 (dq, J=20.2, 6.5 Hz, 6H), 4.47-4.41 (m, 2H), 4.01 (t, J=6.1 Hz, 3H), 2.12 (d, J=7.1 Hz, 6H), 2.00 (q, J=7.1 Hz, 2H), 1.27 (dt, J=9.3, 7.1 Hz, 6H) EI-MS m/z: [M+H]⁺ 963.53.

### Example 32: Preparation of Intermediate Compound 199

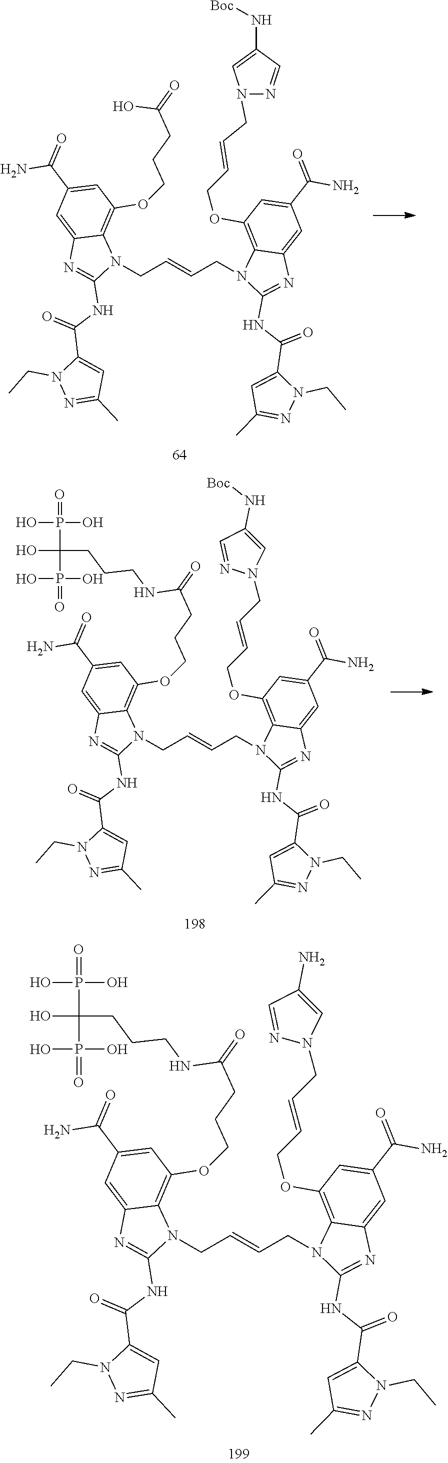 [SMILES: CCn1nc(C)cc1C(=O)Nc1nc2cc(C(N)=O)cc(OC/C=C/Cn3cc(NC(=O)OC(C)(C)C)cn3)c2n1C/C=C/Cn1c(NC(=O)c2cc(C)nn2CC)nc2cc(C(N)=O)cc(OCCCC(=O)O)c21 ; CCn1nc(C)cc1C(=O)Nc1nc2cc(C(N)=O)cc(OC/C=C/Cn3cc(NC(=O)OC(C)(C)C)cn3)c2n1C/C=C/Cn1c(NC(=O)c2cc(C)nn2CC)nc2cc(C(N)=O)cc(OCCCC(=O)NCCCC(O)(P(=O)(O)O)P(=O)(O)O)c21 ; CCn1nc(C)cc1C(=O)Nc1nc2cc(C(N)=O)cc(OC/C=C/Cn3cc(N)cn3)c2n1C/C=C/Cn1c(NC(=O)c2cc(C)nn2CC)nc2cc(C(N)=O)cc(OCCCC(=O)NCCCC(O)(P(=O)(O)O)P(=O)(O)O)c21]

### Preparation of Intermediate Compound 198

To a solution of Intermediate Compound 64 (90 mg, 0.08 mmol) in N,N-dimethylformamide (10 mL). were added alendronic acid (80 mg, 0.32 mmol), N,N,N′,N′-tetramethyl-O-(1H-benzotriazol-1-yl)uronium hexafluorophosphate (HBTU, 91 mg, 0.24 mmol), and triethylamine (0.04 mL, 0.32 mmol) under nitrogen. After stirred at room temperature for 3 days, the reaction solution was concentrated under reduced pressure. The resulting residue was purified by HPLC to afford Intermediate Compound 198 (32 mg, 31%). EI-MS m/z: [M+H]⁺ 1262.42.

### Preparation of Intermediate Compound 199

To a solution of Intermediate Compound 198 (32 mg, 0.02 mmol) in dichloromethane (3 mL) was added trifluoroacetic acid (1 mL) at 0° C. under nitrogen. After stirred at room temperature for 0.5 hours. The reaction solution was concentrated under reduced pressure. The resulting residue was purified by HPLC to afford Intermediate Compound 199 (14 mg, 47%). EI-MS m/z: [M+H]⁺ 1162.53.

### Preparation Example 4: Preparation of Intermediate Compound 204

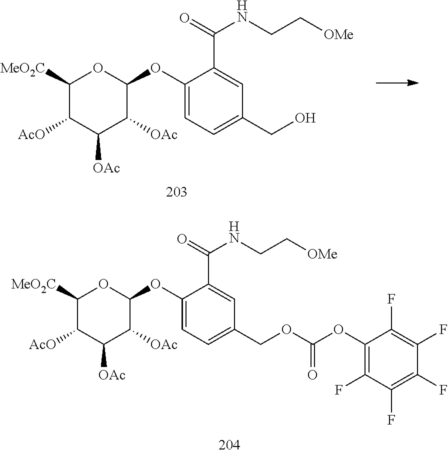 [SMILES: COCCNC(=O)c1cc(CO)ccc1O[C@@H]1O[C@H](C(=O)OC)[C@@H](OC(C)=O)[C@H](OC(C)=O)[C@H]1OC(C)=O ; COCCNC(=O)c1cc(COC(=O)Oc2c(F)c(F)c(F)c(F)c2F)ccc1O[C@@H]1O[C@H](C(=O)OC)[C@@H](OC(C)=O)[C@H](OC(C)=O)[C@H]1OC(C)=O]

### Preparation of Intermediate Compound 204

To a solution of Intermediate Compound 203 (2.0 g, 2.74 mmol, compound 203 was prepared according to the method described in the International patent publication No. WO 2018/182341 A1) in N,N-dimethylformamide (10 mL) were added bis(pentafluorophenyl)carbonate (881 mg, 2.23 mmol) and N,N-diisopropylethylamine (0.66 mL, 3.72 mmol) at 0° C. under nitrogen. After stirred at room temperature for 17 hours, the reaction mixture was diluted with ethyl acetate (100 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 204 (1.38 g, 98%).

¹H-NMR (400 MHz, CDCl₃) δ 8.14 (s, 1H), 7.52 (d, J=8.8 Hz, 2H), 7.10 (d, J=8.4 Hz, 1H), 5.41-5.30 (m, 4H), 4.25-4.18 (m, 1H), 3.73 (s, 4H), 3.59 (s, 3H), 3.43 (d, J=1.4 Hz, 3H), 2.06 (d, J=2.0 Hz, 9H). EI-MS m/z: [M+H]⁺ 751.96.

### Preparation Example 5: Preparation of Intermediate Compound 207

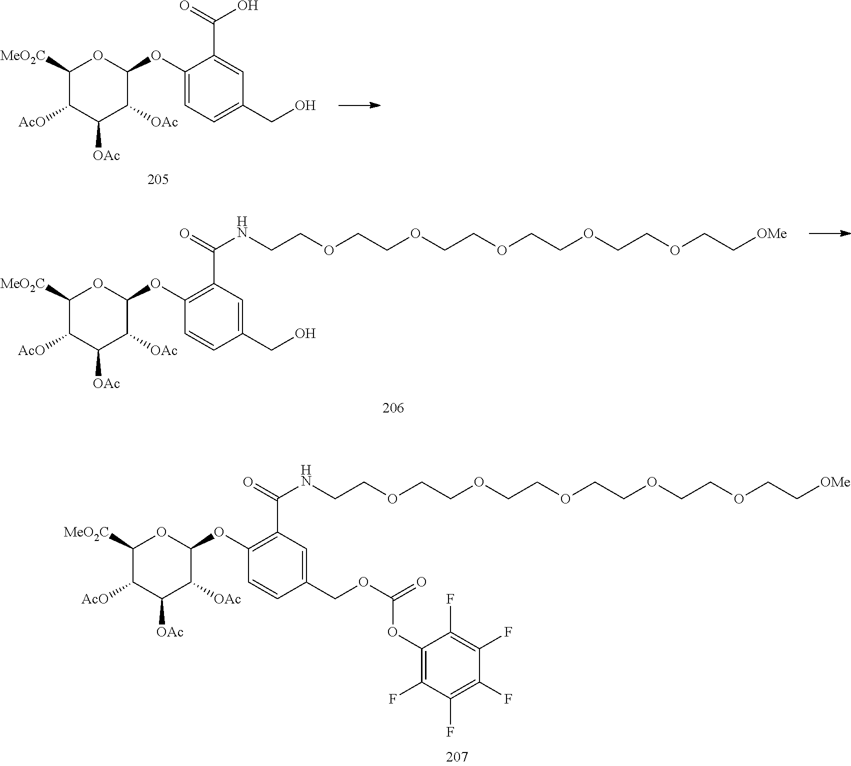 [SMILES: COC(=O)[C@H]1O[C@@H](Oc2ccc(CO)cc2C(=O)O)[C@H](OC(C)=O)[C@@H](OC(C)=O)[C@@H]1OC(C)=O ; COCCOCCOCCOCCOCCOCCNC(=O)c1cc(CO)ccc1O[C@@H]1O[C@H](C(=O)OC)[C@@H](OC(C)=O)[C@H](OC(C)=O)[C@H]1OC(C)=O ; COCCOCCOCCOCCOCCOCCNC(=O)c1cc(COC(=O)Oc2c(F)c(F)c(F)c(F)c2F)ccc1O[C@@H]1O[C@H](C(=O)OC)[C@@H](OC(C)=O)[C@H](OC(C)=O)[C@H]1OC(C)=O]

### Preparation of Intermediate Compound 206

To a solution of Intermediate Compound 205 (162 mg, 0.33 mmol, compound 205 was prepared according to the method described in the International patent publication No. WO 2018/182341 A1) in N,N-dimethylformamide (3 mL) were added 2,5,8,11,14,17-hexaoxanonadecan-19-amine (100 mg, 0.33 mmol), N,N,N′,N′-tetramethyl-O-(1H-benzotriazol-1-yl)uronium hexafluorophosphate (HBTU, 154 mg, 0.41 mmol), and N,N-diisopropylethylamine (0.12 mL, 0.68 mmol) at 0° C. under nitrogen. After stirred at room temperature for 2 hours, the reaction solution was diluted with ethyl acetate (50 mL) and then washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 206 (185 mg, 72%). EI-MS m/z: [M+Na]⁺ 784.18, [M+H]⁺ 762.17.

### Preparation of Intermediate Compound 207

To a solution of Intermediate Compound 206 (185 mg, 0.24 mmol) in dichloromethane (5 mL) were added bis(pentafluorophenyl)carbonate (115 mg, 0.29 mmol) and N,N-diisopropylethylamine (0.06 ml, 0.36 mmol) at 0° C. under nitrogen. After stirred at room temperature for 5 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 207 (92 mg, 39%). EI-MS m/z: [M+H]⁺ 972.13.

### Example 33: Preparation of Compound 214

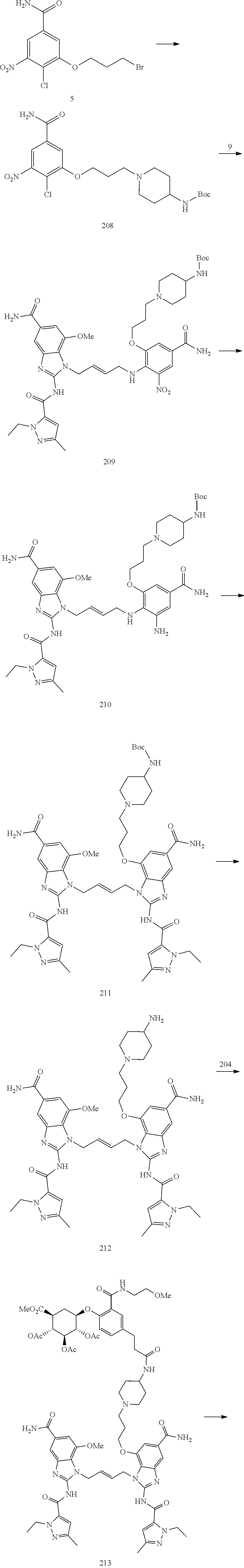

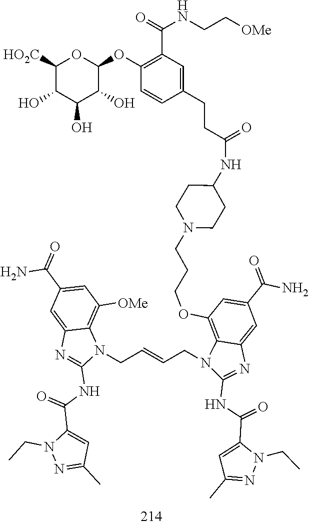 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(NC(=O)CCc4ccc(O[C@@H]5O[C@H](C(=O)O)[C@@H](O)[C@H](O)[C@H]5O)c(C(=O)NCCOC)c4)CC3)c21]

### Preparation of Intermediate Compound 208

To a solution of t-butyl piperidin-4-ylcarbamate (1.2 g, 5.99 mmol) in N,N-dimethylformamide (15 mL) were added compound 5 (2.2 g, 6.59 mmol) and then cesium carbonate (2.14 g, 6.59 mmol) at room temperature under nitrogen. After stirred for 16 hours, the reaction solution was added with distilled water (100 mL) and extracted with ethyl acetate (200 mL×3). The organic layer was dried over anhydrous sodium sulfate and filtered. The filtrate was concentrated under reduced pressure. The resulting residue was purified by column chromatography to afford Intermediate Compound 208 (1.92 g, 70%).

¹H-NMR (400 MHz, CDCl₃) δ 7.69 (s, 2H), 4.43 (m, 1H), 4.22 (t, J=6.4 Hz, 2H), 3.46 (m, 1H), 2.85-2.82 (m, 2H), 2.53 (t, J=7.2 Hz, 2H), 2.12-2.01 (m, 4H), 1.94-1.91 (m, 2H), 1.44-1.42 (m, 10H). EI-MS m/z: [M+H]⁺ 457.52.

### Preparation of Intermediate Compound 209

To a solution of Intermediate Compound 208 (1.30 g, 2.85 mmol) and compound 9 (2.75 g, 5.69 mmol) in n-butanol (30 mL) was added N,N-diisopropylethylamine (2.48 mL, 14.22 mmol). After stirred at 120° C. for 48 hours, the reaction mixture was cooled to room temperature and concentrated under reduced pressure. The reaction mixture was diluted with acetonitrile and diethyl ether to precipitate a solid and then filtered. The solid was dried to afford Intermediate Compound 209 (2.48 g, 84%), which was used without further purification. EI-MS m/z: [M+H]⁺ 832.09, [M/2+H]⁺416.82.

### Preparation of Intermediate Compound 210

To a solution of Intermediate Compound 209 (2.48 g, 2.39 mmol) in methanol (50 mL) were added sodium hydrosulfite (Na₂S₂O₄, 4.17 g, 23.95 mmol) in distilled water (30 mL) and ammonia solution (28˜30% ammonia, 4.6 mL, 59.9 mmol). After stirred at room temperature for 2.5 hours, the precipitate was filtered through Celite and washed with methanol. The filtrate was concentrated under reduced pressure. The resulting residue was purified by reverse phase column chromatography to afford Intermediate Compound 210 (1.4 g, 73%). EI-MS m/z: [M+H]⁺ 802.25.

### Preparation of Intermediate Compound 211

To a solution of Intermediate Compound 210 (760 mg, 0.95 mmol) in N,N-dimethylformamide (15 mL) was added compound 2 (241 mg, 1.23 mmol) in N,N-dimethylformamide (5 mL). After stirred at 30 minutes, the reaction solution was added N-(3-dimethylaminopropyl)-N′-ethylcarbodiimide (221 mg, 1.42 mmol) and triethylamine (0.26 mL, 1.89 mmol) at room temperature for 15 hours. The reaction solution was concentrated under reduced pressure. The resulting residue was purified by column chromatography to afford Intermediate Compound 211 (730 mg, 79%). EI-MS m/z: [M+H]⁺ 963.04, [M/2+H]⁺482.36.

### Preparation of Intermediate Compound 212

To a solution of Intermediate Compound 211 (730 mg, 0.76 mmol) in dichloromethane (8 mL) was added trifluoroacetic acid (0.4 mL) at 0° C. under nitrogen. After stirred at room temperature for 0.5 hours. The reaction solution was concentrated under reduced pressure. The resulting residue was purified by HPLC to afford Intermediate Compound 212 (830 mg, 90%).

¹H-NMR (400 MHz, DMSO-d₆) δ 12.85 (s, 1H), 8.00 (s, 2H), 7.66 (d, J=7.2 Hz, 2H), 7.38 (s, 2H), 7.30 (s, 2H), 6.52 (d, J=5.2 Hz, 2H), 5.75 (d, J=20.8 Hz, 2H), 4.90 (s, 4H), 4.53 (d, J=7.8 Hz, 4H), 4.01 (s, 2H), 3.69 (s, 3H), 3.06 (s, 3H), 2.92 (d, J=12.5 Hz, 2H), 2.12 (s, 6H), 2.06 (d, J=13.4 Hz, 2H), 1.68 (d, J=13.8 Hz, 2H), 1.28 (t, J=7.1 Hz, 6H). EI-MS m/z: [M+H]⁺ 863.07, [M/2+H]⁺432.34.

### Preparation of Intermediate Compound 213

To a solution of Intermediate Compound 212 (68 mg, 0.05 mmol) in N,N-dimethylformamide (3 mL) was added compound 11 (38 mg, 0.05 mmol) and N,N′-diisopropylethylamine (0.045 mL, 0.25 mmol). After stirred at room temperature for 15 hours, the reaction solution was concentrated under reduced pressure. The resulting residue was purified by column chromatography to afford Intermediate Compound 213 (60 mg, 89%). EI-MS m/z: [M+Na]⁺1453.26, [M+H]⁺ 1431.27, [M/2+H]⁺716.55.

### Preparation of Intermediate Compound 214

To a solution of Intermediate Compound 213 (60 mg, 0.042 mmol) in methanol (1.5 mL) was added lithium hydroxide monohydrate (7.04 mg, 0.17 mmol) in distilled water (1.5 mL) at −50° C. under nitrogen. After stirred at 0° C. for 3 hours, the reaction mixture was adjusted to pH 4-5 with acetic acid. The reaction solution was concentrated under reduced pressure. The resulting residue was purified by HPLC to afford Intermediate Compound 214 (32 mg, 59%).

### Example 34: Preparation of Compound 221

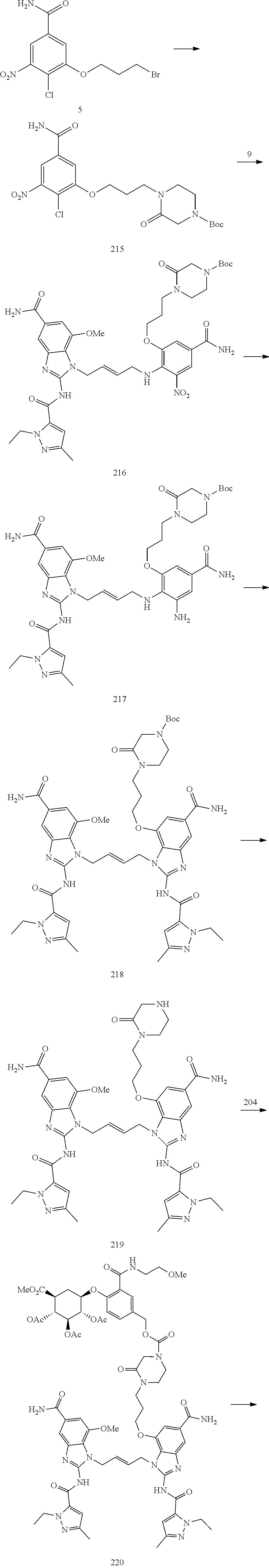

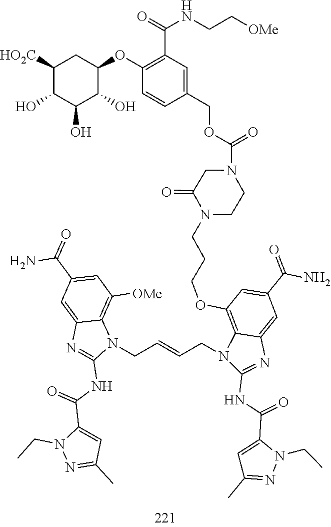 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(O[C@@H]5C[C@H](C(=O)O)[C@@H](O)[C@H](O)[C@H]5O)c(C(=O)NCCOC)c4)CC3=O)c21]

### Preparation of Intermediate Compound 215

To a solution of t-butyl 3-oxopiperazine-1-carboxylate (569 mg, 2.84 mmol) and was dissolved in tetrahydrofuran (12 mL), potassium hydroxide (159 mg, 2.84 mmol) and TBAB (tetrabutylammonium bromide, 152 mg, 0.47 mmol) were added and stirred at room temperature for 30 minutes. The reaction mixture was added Intermediate Compound 5 (800 mg, 2.37 mmol) in tetrahydrofuran (4 mL) at room temperature for 2 hours. The reaction mixture was diluted with ethyl acetate (50 mL) and washed with saturated aqueous ammonium chloride solution (50×2 mL) and dried over anhydrous sodium sulfate then filtered. The filtrate was concentrated under reduced pressure. The resulting residue was purified by column chromatography to afford Intermediate Compound 215 (760 mg, 70.2%).

¹H-NMR (400 MHz, DMSO-d₆) δ 8.27 (s, 1H), 8.05 (d, J=1.7 Hz, 1H), 7.84 (d, J=1.9 Hz, 1H), 7.77 (s, 1H), 4.24 (t, J=6.0 Hz, 2H), 3.86 (s, 2H), 3.53 (dt, J=13.4, 5.8 Hz, 4H), 3.37 (t, J=5.4 Hz, 2H), 2.02 (td, J=11.2, 4.8 Hz, 2H), 1.41 (d, J=3.2 Hz, 9H). EI-MS m/z: [M+H]⁺ 457.08.

### Preparation of Intermediate Compound 216

To a solution of Intermediate Compound 215 (296 mg, 0.45 mmol) were added Intermediate Compound 9 (440 mg, 0.91 mmol) and N,N-diisopropylethylamine (0.43 mL, 2.49 mmol) at room temperature. After stirred at 120° C. for 24 hours, the reaction solution was cooled to room temperature and diluted with dichloromethane (100 mL), methanol (20 mL) and washed with distilled water (50 mL). The organic layer was dried over anhydrous sodium sulfate and filtered. The filtrate was concentrated under reduced pressure to afford Intermediate Compound 216 (crude), which was used without further purification. EI-MS m/z: [M+H]⁺ 832.05, [M/2+H]⁺366.77.

### Preparation of Intermediate Compound 217

To a solution of Intermediate Compound 216 (crude, 0.45 mmol) in methanol (10 mL) were added sodium hydrosulfite (Na₂S₂O₄, 433 mg, 2.48 mmol) and ammonia solution (28˜30% ammonia, 0.55 mL). After stirred at room temperature for 3 hours, the precipitate was filtered through Celite and washed with methanol. The filtrate was concentrated under reduced pressure. The reaction mixture was diluted with dichloromethane (100 mL) and methanol (20 mL) and washed with distilled water (50 mL). The organic layer was dried with anhydrous sodium sulfate and filtered. The filtrate was concentrated under reduced pressure to afford Intermediate Compound 217 (229 mg, 43%), which was used without further purification. EI-MS m/z: [M+H]⁺ 802.11, [M/2+H]⁺365.25.

### Preparation of Intermediate Compound 218

To a solution of Intermediate Compound 217 (229 mg, 0.28 mmol) in N,N-dimethylformamide (4 mL) was added Intermediate Compound 2 (83.6 mg, 0.42 mmol) in N,N-dimethylformamide (1 mL) at −0° C. After stirred for 30 minutes, the traction solution was added N-(3-dimethylaminopropyl)-N′-ethylcarbodiimide (88.6 mg, 0.57 mmol) and triethylamine (0.09 mL, 0.63 mmol) at room temperature for 19 hours. The reaction mixture was concentrated under reduced pressure. The resulting residue was purified by column chromatography to afford Intermediate Compound 218 (110 mg, 40%). EI-MS m/z: [M+H]⁺ 963.19, [M/2+H]⁺482.31.

### Preparation of Intermediate Compound 219

To a solution of Intermediate Compound 218 (50 mg) in dichloromethane (1.4 mL) was added trifluoroacetic acid (0.6 mL) at 0° C. under nitrogen. After stirred at room temperature for 40 minutes, the reaction solution was concentrated under reduced pressure. The resulting residue was purified by HPLC to afford Intermediate Compound 219 (33 mg, 52%).

¹H-NMR (400 MHz, DMSO-d₆) δ 12.81 (s, 1H), 9.09 (s, 2H), 7.97 (d, J=19.3 Hz, 2H), 7.64 (s, 2H), 7.36 (d, J=13.6 Hz, 2H), 7.31 (s, 1H), 7.25 (s, 1H), 6.55 (s, 1H), 6.52 (s, 1H), 5.86 (d, J=15.9 Hz, 1H), 5.76 (d, J=15.8 Hz, 1H), 4.96 (s, 2H), 4.88 (d, J=4.9 Hz, 2H), 4.53 (t, J=7.9 Hz, 4H), 3.95 (s, 2H), 3.71 (s, 3H), 3.65 (s, 2H), 2.12 (d, J=5.9 Hz, 6H), 1.75 (s, 2H), 1.27 (q, J=7.7 Hz, 6H). EI-MS m/z: [M+H]⁺ 863.09, [M/2+H]⁺ 432.41.

### Preparation of Intermediate Compound 220

To a solution of Intermediate Compound 219 (20 mg, 0.017 mmol) in N,N-dimethylformamide (2 mL) was added compound 11 (12.3 mg, 0.05 mmol) and N,N-diisopropylethylamine (0.014 mL, 0.083 mmol). After stirred at room temperature for 15 hours, the reaction solution was concentrated under reduced pressure and purified by column chromatography to afford Intermediate Compound 220 (23 mg, 97%). EI-MS m/z: [M+Na]⁺ 1453.19, [M+H]⁺ 1431.19, [M/2+H]⁺716.48.

### Preparation of Compound 221

To a solution of Intermediate Compound 220 (23 mg, 0.016 mmol) in methanol (1 mL) was added lithium hydroxide monohydrate (1.67 mg, 0.06 mmol) in distilled water (1 mL) at −50° C. under nitrogen. After stirred at 0° C. for 3 hours, the reaction mixture was adjusted to pH 4-5 with acetic acid. The reaction solution was concentrated under reduced pressure. The resulting residue was purified by HPLC to afford Compound 221 (4.4 mg, 21%).

¹H-NMR (400 MHz, DMSO-d₆) δ 12.80 (s, 1H), 8.31 (s, 1H), 7.95 (d, J=13.8 Hz, 2H), 7.82 (s, 1H), 7.64 (d, J=4.1 Hz, 2H), 7.52 (d, J=8.5 Hz, 1H), 7.37-7.21 (m, 5H), 6.57 (s, 1H), 6.50 (s, 1H), 5.90-5.80 (m, 1H), 5.76 (d, J=15.6 Hz, 1H), 5.15 (d, J=6.8 Hz, 1H), 5.08 (s, 2H), 4.96 (s, 2H), 4.88 (s, 1H), 4.51 (dt, J=21.9, 7.3 Hz, 4H), 3.97 (d, J=9.5 Hz, 1H), 3.87 (d, J=17.5 Hz, 4H), 3.69 (s, 3H), 3.52 (s, 4H), 3.46 (d, J=3.7 Hz, 3H), 3.28 (s, 2H), 3.19 (s, 1H), 2.11 (d, J=15.9 Hz, 6H), 1.68 (s, 2H), 1.27 (dt, J=21.1, 7.0 Hz, 6H). EI-MS m/z: [M+H]⁺ 1291.07, [M+H]⁺ 646.39.

### Example 35: Preparation of Compound 230

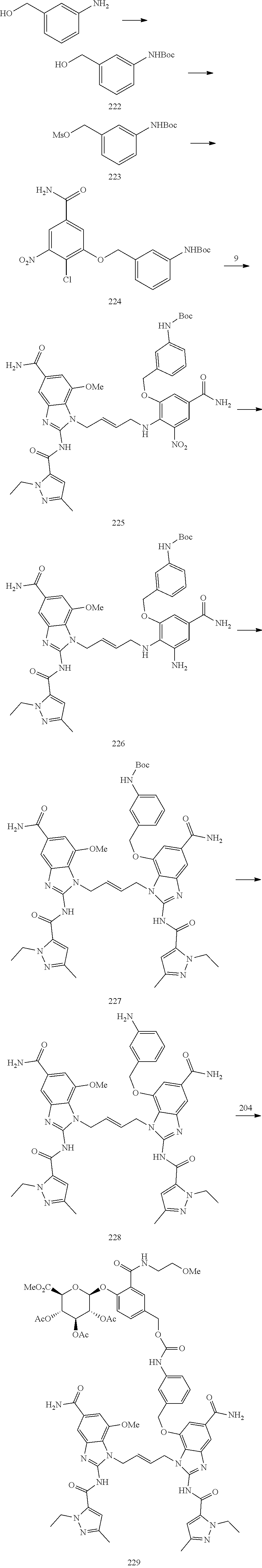

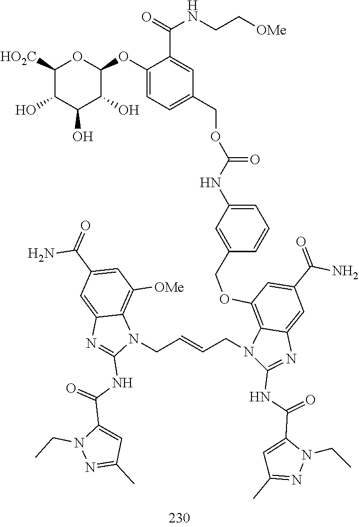 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCc3cccc(NC(=O)OCc4ccc(O[C@@H]5O[C@H](C(=O)O)[C@@H](O)[C@H](O)[C@H]5O)c(C(=O)NCCOC)c4)c3)c21]

### Preparation of Intermediate Compound 222

To a solution of 3-aminobenzyl alcohol (800 mg, 6.49 mmol) in tetrahydrofuran (9.3 mL) was added di-t-butyl dicarbonate (1.64 mL, 7.14 mmol). After stirred at room temperature for 24 hours, the reaction solution was concentrated under reduced pressure. The resulting residue was purified by column chromatography to afford Intermediate Compound 222 (1.45 g, 99%).

¹H-NMR (400 MHz, CDCl₃) δ 7.44 (s, 1H), 7.28 (d, J=8.0 Hz, 1H), 7.22 (d, J=8.1 Hz, 1H), 7.04 (d, J=7.3 Hz, 1H), 6.50 (s, 1H), 4.66 (s, 2H), 1.52 (s, 9H).

### Preparation of Intermediate Compound 223

To a solution of Intermediate Compound 222 (700 mg, 3.13 mmol) in dichloromethane (5 mL) were added triethylamine (0.65 mL, 4.70 mmol) and methanesulfonyl chloride (0.29 mL, 3.76 mmol) at 0° C. After stirred at 0° C. for 3 hours, the reaction solution was concentrated under reduced pressure. The resulting residue was purified by column chromatography to afford Intermediate Compound 223 (508 mg, 53%).

¹H-NMR (400 MHz, CDCl₃) δ 9.45 (s, 1H), 7.61 (s, 1H), 7.41 (d, J=8.2 Hz, 1H), 7.29 (d, J=16.0 Hz, 1H), 7.03 (d, J=7.5 Hz, 1H), 5.19 (s, 2H), 3.24-3.19 (m, 3H), 1.48 (s, 9H).

### Preparation of Intermediate Compound 224

To a solution of Intermediate Compound 4 (300 mg, 1.38 mmol) in N,N-dimethylformamide (3 mL) were added potassium carbonate (287 mg, 2.08 mmol) and compound 223 (500.9 mg, 1.66 mmol). After stirred at 50° C. for 1 hours, the reaction solution was diluted with ethyl acetate (50 mL) and washed with distilled water (50 mL). The organic layer was dried over anhydrous sodium sulfate and filtered the filtrate was concentrated under reduced pressure to afford Intermediate Compound 224 (487 mg, 83%), which was used without further purification.

¹H-NMR (400 MHz, DMSO-d₆) δ 9.44 (s, 1H), 8.27 (s, 1H), 8.07 (d, J=1.7 Hz, 1H), 7.98 (d, J=1.8 Hz, 1H), 7.78 (s, 1H), 7.62 (s, 1H), 7.46-7.39 (m, 1H), 7.30 (t, J=7.8 Hz, 1H), 7.09 (d, J=7.5 Hz, 1H), 5.33 (s, 2H), 1.48 (s, 9H). EI-MS m/z: [M+H]⁺ 422.13.

### Preparation of Intermediate Compound 225

To a solution of Intermediate Compound 224 (350 mg, 0.83 mmol) and Intermediate Compound 9 (683 mg, 1.41 mmol) in n-butanol (6 mL) was added N,N-diisopropylethylamine (0.79 mL, 4.56 mmol) at room temperature. After heated to 120° C. and stirred for 40 hours, the reaction solution was cooled to room temperature. The reaction mixture was diluted with dichloromethane (100 mL), methanol (20 mL) and washed with distilled water (50 mL). The organic layer was dried over anhydrous sodium sulfate and filtered and concentrated under reduced pressure. After added with dichloromethane and diethyl ether, the residue solid was filtered. The residue solid was dried to afford 225 (crude), which was used without further purification. EI-MS m/z: [M+H]⁺ 797.08.

### Preparation of Intermediate Compound 226

After dissolving Intermediate Compound 225 (crude, 0.83 mmol) in methanol (14 mL) and distilled water (2 mL) were added aqueous ammonia solution (28˜30%, 1.05 mL) and sodium hydrosulfite (Na₂S₂O₄, 1.05 g, 6.02 mmol) under nitrogen. After stirred at room temperature for 1 hours, the reaction solution was diluted with methanol (50 mL) and then filtered. The filtrate was concentrated under reduced pressure. The reaction mixture was diluted with ethyl acetate (100 mL) and methanol (20 mL) and washed with distilled water (50 mL). The organic layer was dried with anhydrous sodium sulfate and filtered. The filtrate was concentrated under reduced pressure to afford Intermediate Compound 226 (crude), which was used without further purification. EI-MS m/z: [M+H]⁺ 767.08.

### Preparation of Intermediate Compound 227

To a solution of intermediate compound 226 (0.83 mmol, crude) in N,N-dimethylformamide (5 mL) was added compound 2 (100 mg, 0.52 mmol) in N,N-dimethylformamide (1 mL) under nitrogen. After stirred at −0° C. for 1 hour, the reaction mixture was added N-(3-dimethylaminopropyl)-N′-ethylcarbodiimide hydrochloride (114 mg, 0.65 mmol) and triethylamine (0.18 mL, 1.29 mmol) at room temperature. The reaction mixture was stirred for 18 hours. The reaction mixture was concentrated under reduced pressure. The resulting residue was purified by column chromatography to afford Intermediate Compound 227 (128 mg, 32%). EI-MS m/z: [M+H]⁺ 928.02.

### Preparation of Intermediate Compound 228

To a solution of Intermediate Compound 227 (50 mg) in dichloromethane (2.4 mL) was added trifluoroacetic acid (0.6 mL) at 0° C. under nitrogen. After stirred at room temperature for 30 minutes, the reaction solution was concentrated under reduced pressure. The resulting residue was purified by HPLC to afford Intermediate Compound 228 (17 mg, 27%).

¹H-NMR (400 MHz, DMSO-d₆) δ 7.97 (s, 1H), 7.66 (d, J=9.3 Hz, 2H), 7.43 (s, 1H), 7.37 (s, 1H), 7.28 (s, 1H), 7.14 (t, J=7.8 Hz, 1H), 7.06 (s, 1H), 6.93 (s, 1H), 6.51 (d, J=7.0 Hz, 2H), 5.85-5.74 (m, 1H), 5.68-5.58 (m, 1H), 5.07 (s, 2H), 4.88 (dd, J=18.8, 5.6 Hz, 4H), 4.54-4.49 (m, 4H), 3.64 (s, 3H), 2.10 (d, J=4.4 Hz, 6H), 1.25 (q, J=7.6 Hz, 6H). EI-MS m/z: [M+H]⁺ 828.07.

### Preparation of Intermediate Compound 229

To a solution of Intermediate Compound 228 (40 mg, 0.03 mmol) in N,N-dimethylformamide (2 mL) was added compound 11 (25 mg, 0.03 mmol), N,N-diisopropylethylamine (0.027 mL, 0.15 mmol) and HOAt (1-Hydroxy-7-azabenzotriazole, 0.9 mg, 0.01 mmol). After stirred at room temperature for 19 hours, the reaction mixture was concentrated under reduced pressure to afford Intermediate Compound 229 (crude), which was used without further purification. EI-MS m/z: [M+H]⁺ 1395.83.

### Preparation of Compound 230

To a solution of Intermediate Compound 229 (81 mg, 0.051 mmol) in methanol (0.7 mL) and tetrahydrofuran (0.7 mL) was added lithium hydroxide monohydrate (10.76 mg, 0.25 mmol) in distilled water (0.9 mL) at −45° C. under nitrogen. After stirred at 0° C. for 1 hours, the reaction mixture was adjusted to pH 4-5 with acetic acid. The reaction solution was concentrated under reduced pressure. The resulting residue was purified by HPLC to afford Compound 230 (3.9 mg, 8.9%).

¹H-NMR (400 MHz, DMSO-d₆) δ 12.81 (s, 3H), 9.81 (s, 1H), 8.29 (s, 1H), 7.96 (s, 2H), 7.88 (s, 1H), 7.66 (d, J=11.3 Hz, 2H), 7.61 (s, 1H), 7.52 (d, J=8.6 Hz, 1H), 7.44 (s, 1H), 7.34 (t, J=9.1 Hz, 3H), 7.25 (d, J=8.6 Hz, 2H), 7.08 (t, J=7.9 Hz, 1H), 6.83 (d, J=7.6 Hz, 1H), 6.49 (d, J=16.8 Hz, 2H), 5.82 (d, J=14.8 Hz, 1H), 5.61 (d, J=15.2 Hz, 2H), 5.15 (d, J=6.8 Hz, 1H), 5.08 (d, J=21.2 Hz, 4H), 4.88 (dd, J=35.7, 5.6 Hz, 4H), 4.49 (dq, J=14.0, 7.2 Hz, 4H), 3.96 (d, J=9.5 Hz, 1H), 3.62-3.28 (m, 15H), 2.09 (d, J=9.3 Hz, 6H), 1.24 (dt, J=14.2, 7.0 Hz, 6H). EI-MS m/z: [M+H]⁺ 1445.20.

### Example 36: Preparation of Compound 239

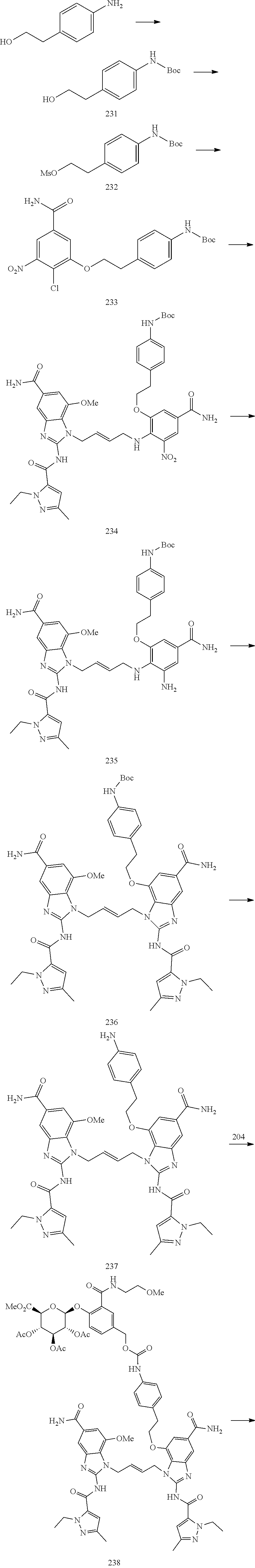

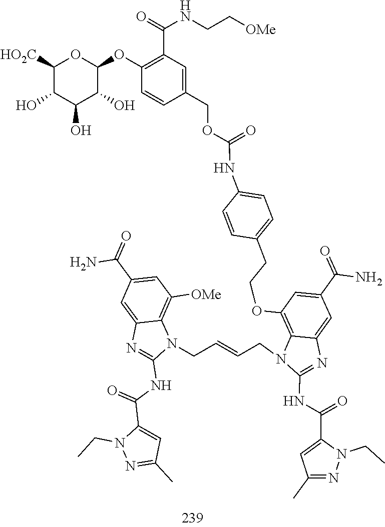 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOC)c4)cc3)c21]

### Preparation of Intermediate Compound 231

To a solution of 4-aminophenethyl alcohol (2 g, 14.96 mmol) in tetrahydrofuran (50 mL) was added di-t-butyl dicarbonate (3.8 mL, 16.46 mmol) at 0° C. After stirred at room temperature for 6 hours, the reaction solution was diluted with dichloromethane (100 mL) and washed with distilled water (50 mL). The organic layer was dried over anhydrous sodium sulfate and filtered. The reaction solution was concentrated under reduced pressure to afford Intermediate Compound 231 (3 g, 85%), which was used without further purification.

¹H-NMR (400 MHz, CDCl₃) δ 7.30 (d, J=8.1 Hz, 2H), 7.15 (d, J=8.2 Hz, 2H), 3.83 (q, J=6.4 Hz, 2H), 2.82 (t, J=6.5 Hz, 2H), 1.52 (s, 9H).

### Preparation of Intermediate Compound 232

To a solution of Intermediate Compound 231 (850 mg, 3.58 mmol) in dichloromethane (15 mL) were added triethylamine (0.75 mL, 5.37 mmol) and methanesulfonyl chloride (0.41 mL, 5.37 mmol) at 0° C. After stirred at room temperature for 2 hours, the reaction solution was diluted with dichloromethane (100 mL) and washed with distilled water (50 mL×2). The organic layer was dried over anhydrous sodium sulfate and filtered. The reaction solution was concentrated under reduced pressure to afford Intermediate Compound 232 (114 mg, crude), which was used without further purification.

### Preparation of Intermediate Compound 233

To a solution of Intermediate Compound 4 (703 mg, 3.25 mmol) in N,N-dimethylformamide (6 mL) were added potassium carbonate (673 mg, 4.88 mmol) and Intermediate Compound 232 (1.1 g, 3.57 mmol). After stirred at 50° C. for 22 hours, the reaction solution was diluted with ethyl acetate (50 mL) and washed with distilled water (15 mL×2) and brine (15 mL). The organic layer was dried over anhydrous sodium sulfate and filtered. The reaction solution was concentrated under reduced pressure to afford Intermediate Compound 233 (1.06 g, 74%).

¹H-NMR (400 MHz, DMSO-d₆) δ 9.21 (s, 1H), 8.22 (s, 1H), 7.97 (d, J=1.7 Hz, 1H), 7.82 (d, J=1.8 Hz, 1H), 7.71 (s, 1H), 7.34 (d, J=8.2 Hz, 2H), 7.19 (d, J=8.3 Hz, 2H), 4.33 (t, J=6.7 Hz, 2H), 3.00 (t, J=6.7 Hz, 2H), 1.42 (s, 9H). EI-MS m/z: [M+H]⁺ 436.13.

### Preparation of Intermediate Compound 234

To a solution of Intermediate Compound 233 (530 mg, 1.22 mmol) and Intermediate Compound 9 (650 mg, 1.58 mmol) in n-butanol (6 mL) was added N,N-diisopropylethylamine (0.95 mL, 5.47 mmol) at room temperature. After stirred at 120° C. for 21 hours, the reaction solution was diluted with dichloromethane (100 mL) and washed with distilled water (50 mL×2). The organic layer was dried over anhydrous magnesium sulfate and filtered. The reaction solution was concentrated under reduced pressure. The resulting residue was purified by column chromatography to afford Intermediate Compound 234 (657 mg, 67%). EI-MS m/z: [M+H]⁺ 436.13.

### Preparation of Intermediate Compound 235

To a solution of Intermediate Compound 234 (373 mg, 0.46 mmol) in methanol (5 mL) were added aqueous ammonia solution (28-30% ammonia, 0.82 mL) and sodium hydrosulfite (Na₂S₂O₄, 800 mg, 46 mmol) under nitrogen. After stirred at room temperature for 30 minutes, the reaction solution was added methanol (50 mL). The resulting solid was filtered and washed with methanol. The filtrate was concentrated and diluted with dichloromethane (100 mL), methanol (20 mL) and washed with distilled water (50 mL). The organic layer was dried over anhydrous sodium sulfate and filtered. The reaction solution was concentrated under reduced pressure to afford Intermediate Compound 235 (360 mg, crude), which was used without further purification. EI-MS m/z: [M+H]⁺ 781.21.

### Preparation of Intermediate Compound 236

To a solution of Intermediate Compound 235 (360 mg, 0.46 mmol) in N,N-dimethylformamide (2 mL) was added compound 2 (98 mg, 0.51 mmol) in N,N-dimethylformamide (1 mL) under nitrogen. After stirred at room temperature for 30 minutes, the reaction mixture were added N-(3-dimethylaminopropyl)-N′-ethylcarbodiimide (0.18 mL, 1.01 mmol) and triethylamine (0.2 mL, 1.38 mmol). After stirred at room temperature for 1.5 hours, the reaction solution was concentrated under reduced pressure. The resulting residue was purified by column chromatography to afford Intermediate Compound 236 (137 mg, 32%). EI-MS m/z: [M+H]⁺ 943.1.

### Preparation of Intermediate Compound 237

To a solution of Intermediate Compound 236 (50 mg) in dichloromethane (2 mL) was added trifluoroacetic acid (0.2 mL) at 0° C. under nitrogen. After stirred at room temperature for 40 minutes, the reaction solution was concentrated under reduced pressure. The resulting residue was purified by HPLC to afford Intermediate Compound 237 (14 mg, 31%).

¹H-NMR (400 MHz, DMSO-d₆) δ 7.91 (d, J=12.5 Hz, 1H), 7.60 (d, J=12.4 Hz, 1H), 7.28 (t, J=7.0 Hz, 2H), 7.14 (d, J=8.0 Hz, 1H), 7.05 (d, J=7.9 Hz, 1H), 6.48 (d, J=14.1 Hz, 1H), 5.72 (d, J=9.4 Hz, 2H), 4.85 (s, 1H), 4.65 (s, 1H), 4.46 (p, J=7.1 Hz, 2H), 4.14 (t, J=6.7 Hz, 1H), 3.67 (s, 2H), 2.79 (t, J=6.7 Hz, 1H), 2.46 (s, 2H), 2.05 (d, J=8.9 Hz, 3H), 1.21 (dt, J=10.6, 7.4 Hz, 3H). EI-MS m/z: [M+H]⁺ 842.14.

### Preparation of Intermediate Compound 238

To a solution of Intermediate Compound 237 (50 mg, 0.06 mmol) in N,N-dimethylformamide (1 mL) was added Intermediate Compound 11 (50 mg, 0.07 mmol), N,N′-diisopropylethylamine (0.05 mL, 0.3 mmol) and 1-Hydroxy-7-azabenzotriazole (HOAt, 1.6 mg, 0.012 mmol). After stirred at room temperature for 2 hours, the reaction mixture was concentrated under reduced pressure to afford Intermediate Compound 238 (114 mg), which was used without further purification. EI-MS m/z: [M+H]⁺ 1410.5.

### Preparation of Compound 239

To a solution of Intermediate Compound 238 (114 mg, 0.08 mmol) in methanol (1 mL) and tetrahydrofuran (1 mL) was added lithium hydroxide monohydrate (20 mg, 0.48 mmol) in distilled water (0.75 mL) at −50° C. under nitrogen. After stirred at 0° C. for 2 hours, the reaction mixture was adjusted to pH 4-5 with acetic acid. The reaction mixture was concentrated under reduced pressure. The resulting residue was purified by HPLC to afford Compound 239 (29 mg, 24%).

¹H-NMR (400 MHz, DMSO-d₆) δ 9.63 (s, 1H), 8.25 (s, 1H), 7.92 (s, 2H), 7.80 (s, 1H), 7.60 (d, J=11.1 Hz, 2H), 7.46 (d, J=8.5 Hz, 1H), 7.34-7.24 (m, 5H), 7.20 (d, J=8.6 Hz, 1H), 7.02 (d, J=8.2 Hz, 2H), 6.47 (s, 2H), 5.10 (d, J=6.7 Hz, 1H), 5.04 (s, 2H), 4.84 (s, 2H), 4.72 (s, 2H), 4.49-4.41 (m, 4H), 4.10 (s, 2H), 3.91 (d, J=9.3 Hz, 1H), 3.67 (s, 3H), 3.23 (s, 2H), 2.74 (s, 2H), 2.04 (d, J=11.7 Hz, 5H), 1.86 (s, 1H), 1.20 (dt, J=12.8, 7.0 Hz, 6H), 0.81 (d, J=11.8 Hz, 1H). EI-MS m/z: [M+H]⁺ 1269.52.

### Example 37: Preparation of Compound 252

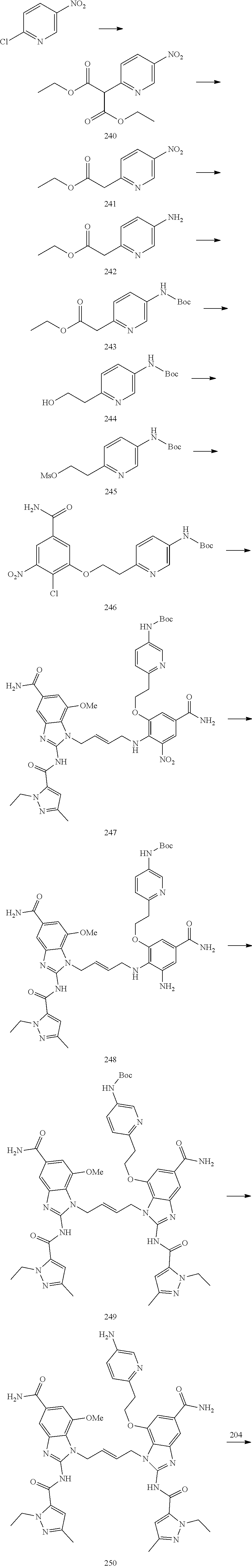

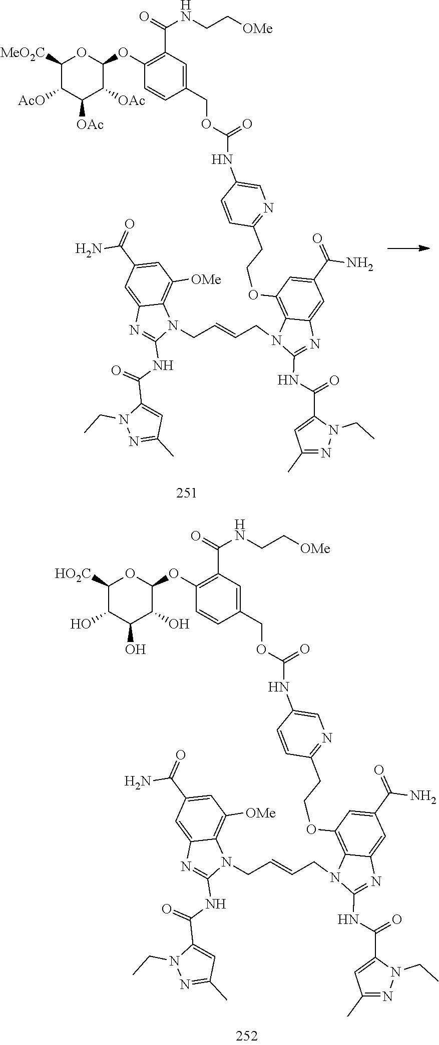 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)OC)[C@@H](OC(C)=O)[C@H](OC(C)=O)[C@H]5OC(C)=O)c(C(=O)NCCOC)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOC)c4)cn3)c21]

### Preparation of Intermediate Compound 240

To a solution of diethyl malonate (2 g, 12.49 mmol) in tetrahydrofuran (50 mL) was added sodium hydride (60% dispersion in mineral oil, 0.9 g, 24.93 mmol) at 0° C. After stirred at 0° C. for 0.5 hours, the reaction solution was added 2-chloro-5-nitropyridine (2.08 g, 13.11 mmol) at room temperature for 18 hours. The reaction mixture was diluted with ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 240 (3.3 g, 94%).

¹H-NMR (400 MHz, CDCl3) δ 9.37 (d, J=2.7 Hz, 1H), 8.51 (dt, J=8.7, 2.4 Hz, 1H), 7.77 (dd, J=8.7, 1.6 Hz, 1H), 5.06 (d, J=1.5 Hz, 1H), 4.35-4.19 (m, 4H), 1.33-1.25 (m, 6H). EI-MS m/z: [M+H]⁺ 283.41.

### Preparation of Intermediate Compound 241

To a solution of Intermediate Compound 240 (3.3 g, 11.69 mmol) in Dimethyl sulfoxide (33 mL) was added sodium chloride (0.72 g, 12.27 mmol) in water (0.3 mL). After stirred at 120° C. for 5 hours, the reaction mixture was diluted with ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 241 (1.27 g, 51%).

¹H-NMR (400 MHz, CDCl₃) δ 9.38 (d, J=2.7 Hz, 1H), 8.46 (dd, J=8.4, 3.0 Hz, 1H), 7.54 (dd, J=8.5, 2.3 Hz, 1H), 4.26-4.16 (m, 2H), 3.98 (d, J=2.3 Hz, 2H), 1.28 (td, J=7.3, 2.3 Hz, 3H). EI-MS m/z: [M+H]⁺ 211.39.

### Preparation of Intermediate Compound 243

To a solution of Intermediate Compound 241 (775 mg, 3.68 mmol) in methanol (15 mL) was added palladium/charcoal (10% wt. Pd/C, 77 mg). After stirred at room temperature under hydrogen balloon for 3 hours, the reaction solution was filtered through Celite. The filtrate was concentrated under reduced pressure. The resulting residue was dissolved in 1,4-dioxane (12 mL) at room temperature and then di-t-butyl dicarbonate (885 mg, 4.06 mmol) was added. After stirred at 100° C. for 17 hours, the reaction solution was concentrated under reduced pressure. The resulting residue was purified by column chromatography to afford Intermediate Compound 243 (906 mg, 87%).

¹H-NMR (400 MHz, CDCl₃) δ 8.36 (d, J=2.6 Hz, 1H), 7.98-7.93 (m, 1H), 7.29-7.19 (m, 1H), 4.17 (qd, J=7.2, 2.0 Hz, 2H), 3.78 (d, J=2.0 Hz, 2H), 1.52 (d, J=2.3 Hz, 9H), 1.25 (td, J=7.2, 2.1 Hz, 3H). EI-MS m/z: [M+H]⁺ 281.30.

### Preparation of Intermediate Compound 244

To a solution of Intermediate Compound 243 (906 mg, 3.23 mmol) in tetrahydrofuran (10 mL) was slowly added Lithium borohydride (1 M in tetrahydrofuran, 9.69 mL, 9.69 mmol) at 0° C. After stirred at room temperature for 4 hours, the reaction solution was diluted with ethyl acetate (50 mL) and washed with distilled water (15 mL×2). The organic layer was dried over anhydrous sodium sulfate and filtered. The reaction solution was concentrated under reduced pressure. The resulting residue was purified by column chromatography to afford Intermediate Compound 244 (294 mg, 38%).

¹H-NMR (400 MHz, CDCl₃) δ 8.65 (s, 1H), 8.16 (s, 1H), 7.43 (d, J=8.6 Hz, 1H), 6.59 (s, 1H), 4.05 (q, J=5.8 Hz, 2H), 3.40 (t, J=6.2 Hz, 2H), 2.65 (bs, 1H), 1.53 (s, 9H). EI-MS m/z: [M+H]⁺ 239.29.

### Preparation of Intermediate Compound 245

To a solution of Intermediate Compound 244 (294 mg, 1.23 mmol) in dichloromethane (6 mL) were added triethylamine (0.22 mL, 1.60 mmol) and methanesulfonyl chloride (0.11 mL, 1.36 mmol) at 0° C. under nitrogen. After stirred at room temperature for 1 hours, the reaction solution was concentrated under reduced pressure. The resulting residue was purified by column chromatography to afford Intermediate Compound 245 (390 mg, 99%).

¹H-NMR (400 MHz, CDCl₃) δ 8.47 (d, J=2.6 Hz, 1H), 7.99 (s, 1H), 7.20 (d, J=8.5 Hz, 1H), 6.83-6.76 (m, 1H), 4.62 (t, J=6.6 Hz, 2H), 3.20 (t, J=6.5 Hz, 2H), 2.92 (s, 3H), 1.53 (s, 9H). EI-MS m/z: [M+H]⁺ 317.57.

### Preparation of Intermediate Compound 246

To a solution of Intermediate Compound 4 (220 mg, 1.02 mmol) in N,N-dimethylformamide (3 mL) were added potassium carbonate (210 mg, 1.52 mmol) and compound 245 (385 mg, 1.22 mmol). After reaction mixture was stirred at 50° C. for 15 hours, then the reaction temperature was raised to 80° C. and stirred for 4 hours. The reaction solution was diluted with ethyl acetate (50 mL) and washed with distilled water (50 mL×2). The organic layer was dried over anhydrous magnesium sulfate and filtered. The filtrate was concentrated under reduced pressure. The resulting residue was purified by column chromatography to afford an Intermediate Compound 246 (270 mg, 60%).

¹H-NMR (400 MHz, DMSO-d₆) δ 9.49 (s, 1H), 8.54 (s, 1H), 8.28 (s, 1H), 8.02 (s, 1H), 7.91 (s, 1H), 7.81 (d, J=8.5 Hz, 1H), 7.76 (s, 1H), 7.30 (d, J=8.5 Hz, 1H), 4.55 (t, J=6.7 Hz, 2H), 3.20 (t, J=6.6 Hz, 2H), 1.47 (d, J=1.8 Hz, 9H). EI-MS m/z: [M+H]⁺ 437.26.

### Preparation of Intermediate Compound 247

To a solution of Intermediate Compound 246 (270 mg, 0.62 mmol) and Intermediate Compound 9 (508 mg, 1.05 mmol) were dissolved in n-butanol (5 mL) and then N,N-diisopropylethylamine (0.59 mL, 3.40 mmol) was added at room temperature. After stirred at 120° C. for 18 hours, The reaction solution was concentrated under reduced pressure. The resulting residue was purified by column chromatography to afford Intermediate Compound 247 (200 mg, 39%). EI-MS m/z: [M+H]⁺ 812.48.

### Preparation of Intermediate Compound 248

To a solution of Intermediate Compound 247 (213 mg, 0.26 mmol) in methanol (7 mL) and distilled water (1 mL) were added ammonia solution (28-30% ammonia, 0.25 mL) and sodium hydrosulfite (Na₂S₂O₄, 456 mg, 2.62 mmol) at −0° C. After stirred at room temperature for 1 hour, the reaction solution was added methanol (50 mL). The resulting solid was filtered and washed with methanol. The filtrate was concentrated under reduced pressure and diluted with acetonitrile. The resulting solid was filtered to afford Intermediate Compound Intermediate Compound 248 (crude), which was used without further purification. EI-MS m/z: [M+H]+ 782.35.

### Preparation of Intermediate Compound 249

To a solution of Intermediate Compound 248 (crude, 0.26 mmol) in N,N-dimethylformamide (3 mL) was added compound 2 (58 mg, 0.30 mmol) in N,N-dimethylformamide (1 mL) at −0° C. After stirred at room temperature for 1 hours, the reaction solution was added N-(3-dimethylaminopropyl)-N′-ethylcarbodiimide (58 mg, 0.37 mmol) and triethylamine (0.10 mL, 0.75 mmol). After stirred at room temperature for 20 hours, the reaction mixture was concentrated under reduced pressure. The resulting residue was purified by column chromatography to afford Intermediate Compound 249 (116 mg, 49%). EI-MS m/z: [M+H]⁺ 943.49.

### Preparation of Intermediate Compound 250

To a solution of Intermediate Compound 249 (50 mg) in dichloromethane (1.6 mL) was added trifluoroacetic acid (0.4 mL) at 0° C. under nitrogen. After stirred at room temperature for 1 hours, the reaction solution was concentrated under reduced pressure. The resulting residue was purified by HPLC to afford Intermediate Compound 250 (25 mg, 36%).

¹H-NMR (400 MHz, DMSO-d₆) δ 7.94 (s, 2H), 7.64 (d, J=10.4 Hz, 2H), 7.54 (q, J=9.0 Hz, 2H), 7.37 (s, 2H), 7.32 (d, J=16.5 Hz, 2H), 6.48 (d, J=26.9 Hz, 2H), 5.69 (s, 2H), 4.86 (s, 2H), 4.76 (s, 2H), 4.48 (p, J=7.0 Hz, 4H), 4.38 (t, J=6.5 Hz, 2H), 3.70 (s, 3H), 2.09 (d, J=7.0 Hz, 6H), 1.23 (q, J=6.4 Hz, 6H). EI-MS m/z: [M+H]⁺ 843.41.

### Preparation of Intermediate Compound 251

To a solution of Intermediate Compound 250 (46 mg, 0.04 mmol) in N,N-dimethylformamide (2 mL) was added Intermediate Compound 11 (29 mg, 0.04 mmol), N,N-diisopropylethylamine (0.03 mL, 0.16 mmol) and HOAt (1-Hydroxy-7-azabenzotriazole (0.9 mg, 0.01 mmol). After stirred at room temperature for 21 hours, the reaction mixture was diluted with ethyl acetate (20 mL), washed with distilled water (8 mL), dried over anhydrous sodium sulfate and filtered. The filtrate was concentrated under reduced pressure to afford Intermediate Compound 251 (crude), which was used without further purification. EI-MS m/z: [M+H]+ 1410.60.

### Preparation of Compound 252

To a solution of Intermediate Compound 251 (crude, 0.04 mmol) in methanol (0.3 mL) and tetrahydrofuran (0.3 mL) was added lithium hydroxide monohydrate (7.45 mg, 0.18 mmol) in distilled water (0.3 mL) at −45° C. under nitrogen. After stirred at 0° C. for 1 hours, the reaction mixture was adjusted to pH 4-5 with acetic acid. The reaction solution was concentrated under reduced pressure. The resulting residue was purified by HPLC to afford Compound 252 (10.5 mg, 23.4% for 2 steps).

¹H-NMR (400 MHz, DMSO) δ 10.04 (s, 1H), 8.60 (s, 1H), 8.28 (s, 1H), 7.95 (s, 1H), 7.87 (s, 2H), 7.63 (d, J=3.7 Hz, 2H), 7.51 (d, J=8.6 Hz, 1H), 7.37-7.28 (m, 4H), 7.28-7.20 (m, 2H), 6.50 (d, J=13.4 Hz, 2H), 5.74 (s, 2H), 5.14 (d, J=11.1 Hz, 3H), 4.87 (s, 2H), 4.74 (s, 2H), 4.49 (d, J=7.6 Hz, 4H), 4.36 (s, 2H), 3.96 (d, J=9.4 Hz, 2H), 3.72-3.46 (m, 13H), 3.38 (d, J=6.3 Hz, 3H), 3.27 (s, 3H), 3.02 (s, 2H), 2.08 (d, J=5.2 Hz, 6H), 1.23 (d, J=6.5 Hz, 6H). EI-MS m/z: [M+H]⁺ 1270.66.

### Example 38: Preparation of Intermediate Compound 254

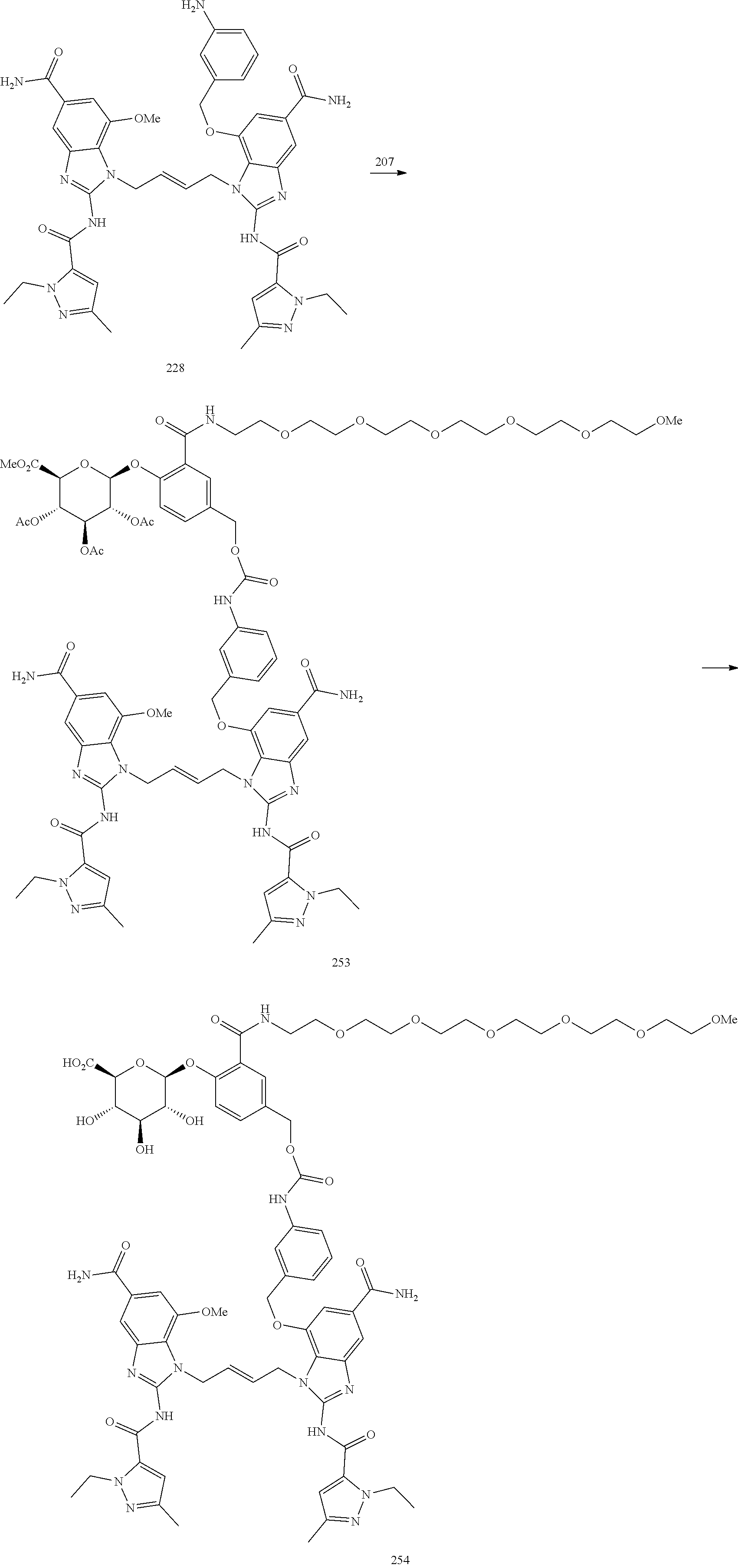

### Preparation of Intermediate Compound 253

To a solution of Intermediate Compound 228 (70 mg, 0.06 mmol) in N,N-dimethylformamide (3 mL) was added compound 207 (45 mg, 0.05 mmol), N,N′-diisopropylethylamine (0.045 mL, 0.25 mmol) and HOAt (1-Hydroxy-7-azabenzotriazole, 2.5 mg, 0.02 mmol). After stirred at room temperature for 15 hours, the reaction solution was concentrated under reduced pressure. The resulting residue was purified by column chromatography to afford Intermediate Compound 253 (74 mg, 98%). EI-MS m/z: [M+Na]⁺ 1637.23, [M+H]⁺ 1615.23, [M/2+H]⁺808.62.

### Preparation of Compound 254

To a solution of Intermediate Compound 253 (74 mg, 0.045 mmol) in methanol (1.5 mL) was added lithium hydroxide monohydrate (7.68 mg, 0.18 mmol) in distilled water (1.5 mL) at −50° C. under nitrogen. After stirred at 0° C. for 3 hours, the reaction mixture was adjusted to pH 4-5 with acetic acid. The reaction solution was concentrated under reduced pressure. The resulting residue was purified by HPLC to afford Compound 254 (29 mg, 43%).

¹H-NMR (400 MHz, DMSO-d₆) δ 9.80 (s, 1H), 8.28 (d, J=5.8 Hz, 1H), 7.95 (s, 2H), 7.87 (d, J=2.3 Hz, 1H), 7.66 (d, J=11.5 Hz, 2H), 7.60 (s, 1H), 7.55-7.48 (m, 1H), 7.44 (s, 1H), 7.32 (d, J=8.3 Hz, 3H), 7.25 (d, J=8.5 Hz, 2H), 7.08 (t, J=7.8 Hz, 1H), 6.83 (d, J=7.6 Hz, 1H), 6.48 (d, J=16.9 Hz, 2H), 5.81 (d, J=15.3 Hz, 1H), 5.65-5.56 (m, 1H), 5.13 (d, J=6.9 Hz, 1H), 5.10 (s, 2H), 5.05 (s, 2H), 4.92 (s, 1H), 4.83 (d, J=5.6 Hz, 2H), 4.49 (dd, J=13.1, 6.9 Hz, 4H), 3.96 (d, J=9.5 Hz, 1H), 3.61 (s, 2H), 3.51 (d, J=22.0 Hz, 20H), 3.22 (d, J=1.5 Hz, 3H), 2.08 (d, J=9.3 Hz, 6H), 1.23 (dt, J=14.2, 7.1 Hz, 6H). EI-MS m/z: [M+H]⁺ 1476.67, [M/2+H]⁺739.23.

### Example 39: Preparation of Intermediate Compound 262

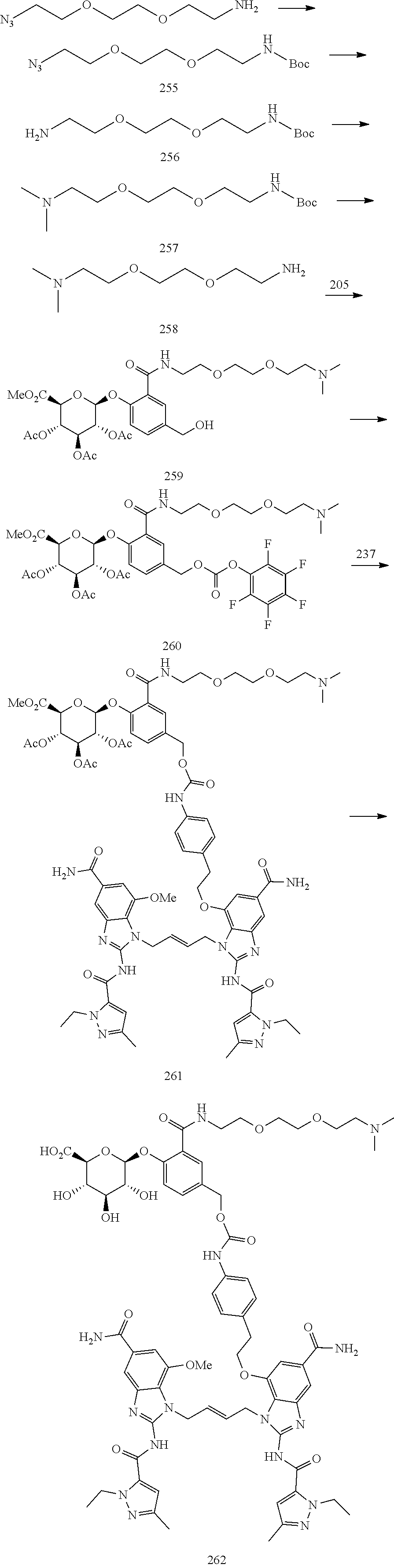

### Preparation of Intermediate Compound 255

To a solution of 2-(2-(2-azidoethoxy)ethoxy)ethan-1-amine (2 g, 11.48 mmol) in dichloromethane (20 mL) were added di-t-butyl dicarbonate (3.16 mL, 13.78 mmol) and triethylamine (3.16 mL 13.78 mmol) at 0° C. The reaction mixture was stirred at room temperature for 5 hours. The reaction mixture was diluted with ethyl acetate (20 mL) and washed with aqueous 1N hydrochloride solution (20 mL). The organic layer was dried over anhydrous sodium sulfate and filtered. The filtrate was concentrated under reduced pressure to afford Intermediate Compound 255 (3 g, crude), which was used without further purification.

¹H-NMR (400 MHz, CDCl₃)) δ 5.02 (br, 1H), 3.69-3.59 (m, 6H), 3.56 (t, J=4.8 Hz, 2H), 3.42 (t, J=4.8 Hz, 2H), 3.33 (d, J=4.4 Hz, 2H), 1.45 (s, 9H).

### Preparation of Intermediate Compound 256

To a solution of Intermediate Compound 255 (1.9 g, 6.93 mmol) in methanol (20 mL) was added palladium/charcoal (10% wt. Pd/C, 190 mg). After stirred at room temperature under hydrogen balloon for 3 hours, the reaction solution was passed through Celite. The filtrate was concentrated under reduced pressure to afford Intermediate Compound 256 (1.7 g, crude), which was used without further purification.

¹H-NMR (400 MHz, CDCl₃) δ 5.19 (br, 1H), 3.63-3.54 (m, 6H), 3.32 (s, 2H), 2.94 (s, 2H), 2.60 (s, 2H), 1.44 (s, 9H).

### Preparation of Intermediate Compound 257

To a solution of Intermediate Compound 256 (452 mg, 1.82 mmol) in methanol (10 mL) and distilled water (1 mL) were added paraformaldehyde (175 mg, 3.65 mmol) and sodium cyanoborohydride (251 mg, 4.00 mmol) and Zinc chloride (99 mg, 0.98 mmol). After stirred at room temperature for 16 hours, the reaction mixture was diluted with dichloromethane (20 mL)/methanol (2 mL) and washed with distilled water (10 mL) and dried over anhydrous magnesium sulfate and filtered. The filtrate was concentrated under reduced pressure. The resulting residue was purified by column chromatography to afford Intermediate Compound 257 (207 mg, 41%).

¹H-NMR (400 MHz, CDCl₃) δ 5.20 (br, 1H), 3.61-3.31 (m, 8H), 3.31 (d, J=4.0 Hz, 2H), 2.52 (t, J=5.6 Hz, 2H), 2.27 (s, 6H), 1.44 (s, 9H).

### Preparation of Intermediate Compound 258

To a solution of Intermediate Compound 257 (207 mg, 0.75 mmol) in dichloromethane (2 mL) was added trifluoroacetic acid (0.4 mL) at 0° C. The reaction mixture was raised to room temperature and stirred for 1 hours. The reaction mixture was diluted with dichloromethane/methanol (10/1, 20 mL×2) and washed with aqueous 3N sodium hydroxide solution (5 mL). The organic layer was dried over anhydrous sodium sulfate and filtered. The filtrate was concentrated under reduced pressure to afford Intermediate Compound 258 (105 mg, 80%), which was used without further purification.

¹H-NMR (400 MHz, CDCl₃)) δ 3.62 (s, 4H), 3.58 (t, J=4.4 Hz, 2H), 2.86 (t, J=4.0 Hz, 2H), 2.52 (d, J=4.0 Hz, 2H), 1.67 (br, 2H).

### Preparation of Intermediate Compound 259

To a solution of Intermediate Compound 258 (240 mg, 0.49 mmol) in N,N-dimethylformamide (10 mL) was added Intermediate Compound 205 (105 mg, 0.59 mmol), N,N,N′,N′-Tetramethyl-O-(1H-benzotriazol-1-yl)uronium hexafluorophosphate (HBTU, 244 mg, 0.64 mmol), N,N-diisopropylethylamine (0.19 mL, 0.99 mmol) at −0° C. After raised to room temperature and stirred under nitrogen for 3 hours. The reaction mixture was diluted with ethyl acetate (50 mL) and washed with distilled water (50 mL). The organic layer was dried over anhydrous sodium sulfate and filtered. The filtrate was concentrated under reduced pressure to afford Intermediate Compound 259 (380 mg, crude), which was used without further purification. EI-MS m/z: [M+H]⁺ 643.41.

### Preparation of Intermediate Compound 260

To a solution of Intermediate Compound 259 (490 mg, 0.76 mmol) in dichloromethane (10 mL) was added bis(pentafluorophenyl)carbonate (361 mg, 0.92 mmol) and N,N-diisopropylethylamine (0.4 ml, 0.29 mmol) at 0° C. After raised to room temperature and stirred under nitrogen for 5 hours. The reaction mixture was diluted with dichloromethane (15 mL) and washed with distilled water (15 mL×2). The organic layer was dried over anhydrous sodium sulfate and filtered. The filtrate was concentrated under reduced pressure to afford Intermediate Compound 260 (400 mg, crude), which was used without further purification. EI-MS m/z: [M+H]⁺ 853.39.

### Preparation of Intermediate Compound 261

To a solution of Intermediate Compound 237 (60 mg, 0.05 mmol) in N,N-dimethylformamide (3 mL) was added compound 260 (52 mg, 0.06 mmol), N,N-diisopropylethylamine (0.04 mL, 0.26 mmol) and HOAt (1-Hydroxy-7-azabenzotriazole, 1.4 mg, 0.01 mmol). After stirred at room temperature for 2 hours, the reaction mixture was concentrated under reduced pressure to afford Intermediate Compound 261 (80 mg, crude), which was used without further purification. EI-MS m/z: [M+1/2]⁺ 756.82, [M+H]⁺ 1511.96.

### Preparation of Compound 262

To a solution of Intermediate Compound 261 (crude 80 mg, 0.053 mmol) in methanol (1 mL) and tetrahydrofuran (1 mL) was added lithium hydroxide monohydrate (22 mg, 0.53 mmol) in distilled water (1 mL) at −50° C. under nitrogen. After stirred at 0° C. for 3 hours, the reaction mixture was adjusted to pH 4-5 with acetic acid. The reaction solution was concentrated under reduced pressure. The resulting residue was purified by HPLC to afford Compound 262 (12.2 mg, 17%). EI-MS m/z: [M/2+H]⁺ 686.25.

### Example 40: Preparation of Compound 270

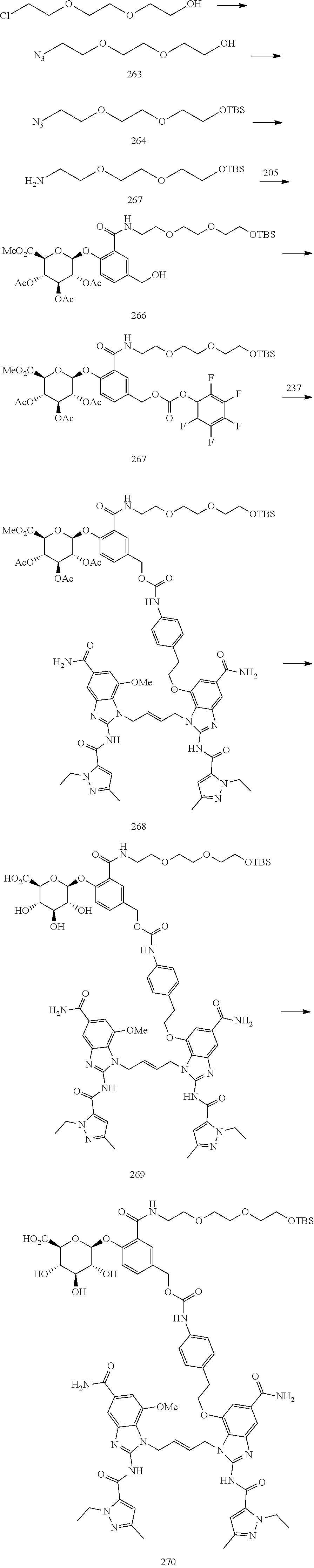

### Preparation of Intermediate Compound 263

To a solution of 2-(2-(2-chloroethoxy)ethoxy)ethan-1-ol (10 g, 59.3 mmol) in N,N-dimethylformamide (30 mL) was added sodium azide (4.63 g, 71.2 mmol) at 0° C. After stirred at 100° C. for 16 hours, The reaction mixture was diluted with chloroform (50 mL) and washed with distilled water (15 mL). The organic layer was dried over anhydrous sodium sulfate and filtered. The filtrate was concentrated under reduced pressure to afford Intermediate Compound 263 (10.8 g, crude), which was used without further purification.

¹H-NMR (400 MHz, CDCl₃) δ 3.74 (t, J=5.0 Hz, 2H), 3.71-3.65 (m, 6H), 3.64-3.59 (m, 2H), 3.40 (t, J=5.0 Hz, 2H), 2.34 (t, J=6.2 Hz, 1H). EI-MS m/z: [M+Na]⁺ 198.25.

### Preparation of Intermediate Compound 264

To a solution of Intermediate Compound 263 (2 g, 12.49 mmol) in tetrahydrofuran (50 mL) was added sodium hydride (60% dispersion in mineral oil, 501 mg, 20.9 mmol) at 0° C. The reaction mixture was stirred at 0° C. for 10 minutes. t-butyl dimethylchlorosilane (2.3 ml, 12.6 mmol) was added to the reaction mixture and stirred at room temperature for 15 hours. The reaction mixture was diluted with ethyl acetate (20 mL×3) and washed with brine (15 mL) and dried over anhydrous sodium sulfate and filtered. The filtrate was concentrated under reduced pressure. The resulting residue was purified by column chromatography to afford Intermediate Compound 264 (3.05 g, 92%).

¹H-NMR (400 MHz, CDCl₃) δ 3.77 (t, J=5.3 Hz, 2H), 3.72-3.62 (m, 6H), 3.57 (t, J=5.4 Hz, 2H), 3.39 (t, J=5.1 Hz, 2H), 0.90 (s, 9H), 0.07 (s, 6H). EI-MS m/z: [M+Na]⁺ 312.36, [M+H]⁺ 290.38.

### Preparation of Intermediate Compound 265

To a solution of Intermediate Compound 264 (3.05 g, 10.5 mmol) in tetrahydrofuran (30 mL) was added triphenylphosphine (4.63 g, 71.2 mmol). After stirred at 100° C. for 16 hours, the reaction mixture was concentrated under reduced pressure to afford Intermediate Compound 265 (1.5 g, 54%), which was used without further purification.

¹H-NMR (400 MHz, CDCl₃) δ 3.77 (t, J=5.5 Hz, 2H), 3.68-3.65 (m, 2H), 3.64-3.61 (m, 2H), 3.56 (t, J=5.4 Hz, 2H), 3.51 (t, J=5.2 Hz, 2H), 2.86 (t, J=5.3 Hz, 2H), 0.89 (s, 9H), 0.07 (s, 6H). EI-MS m/z: [M+H]⁺ 264.38.

### Preparation of Intermediate Compound 266

To a solution of Intermediate Compound 265 (2.20 g, 4.54 mmol) in N,N-dimethylformamide (12 mL) was added Intermediate 205 (1.44 g, 5.45 mmol), N,N,N′,N′-Tetramethyl-O-(1H-benzotriazol-1-yl)uronium hexafluorophosphate (HBTU, 2.24 g, 5.90 mmol), N,N-diisopropylethylamine (1.6 mL, 9.08 mmol) at −0° C. After raised to room temperature and stirred under nitrogen for 3 hours. The reaction mixture was diluted with ethyl acetate (50 mL) and washed with saturated aqueous sodium hydrogen carbonate (50 mL) and distilled water (50 mL). The organic layer was dried over anhydrous sodium sulfate and filtered. The filtrate was concentrated under reduced pressure. The resulting residue was purified by column chromatography to afford the Intermediate Compound 266 (830 mg, crude).

¹H-NMR (400 MHz, CDCl₃) δ 7.96 (s, 1H), 7.46 (d, J=8.5 Hz, 1H), 7.36 (d, J=6.0 Hz, 1H), 7.04 (d, J=8.4 Hz, 1H), 5.43-5.22 (m, 4H), 4.67 (s, 2H), 4.20 (d, J=9.3 Hz, 1H), 3.77-3.72 (m, 6H), 3.69-3.65 (m, 6H), 3.55 (t, J=5.5 Hz, 3H), 2.05 (s, 9H), 0.05 (d, J=1.4 Hz, 6H). EI-MS m/z: [M+Na]⁺752.41, [M+H]⁺ 730.44.

### Preparation of Intermediate Compound 267

To a solution of Intermediate Compound 266 (830 mg, 1.14 mmol) in dichloromethane (5 mL) was added bis(pentafluorophenyl)carbonate (538 mg, 1.36 mmol) and N,N-diisopropylethylamine (0.59 ml, 3.41 mmol) at 0° C. After raised to room temperature and stirred under nitrogen for 3 hours. The reaction mixture was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford the Intermediate Compound 267 (270 mg, 25%).

¹H-NMR (400 MHz, CDCl₃) δ 8.11 (d, J=2.6 Hz, 1H), 7.51 (d, J=8.4 Hz, 1H), 7.37 (s, 1H), 7.09 (d, J=8.5 Hz, 1H), 5.45-5.31 (m, 3H), 5.30-5.27 (m, 3H), 4.22 (d, J=9.1 Hz, 1H), 3.80-3.64 (m, 13H), 3.56 (t, J=5.6 Hz, 3H), 2.06 (s, 9H), 0.91-0.86 (m, 9H), 0.05 (t, J=1.2 Hz, 6H). EI-MS m/z: [M+H]⁺ 940.43.

### Preparation of Intermediate Compound 268

To a solution of Intermediate Compound 267 (75.4 mg, 0.06 mmol) in N,N-dimethylformamide (2 mL) was added compound 237 (65.8 mg, 0.07 mmol), N,N-diisopropylethylamine (0.06 mL, 0.32 mmol) and HOAt (1-Hydroxy-7-azabenzotriazole, 1.7 mg, 0.01 mmol). After stirred at room temperature for 2 hours, the reaction mixture was concentrated under reduced pressure to afford Intermediate Compound 268, which was used without further purification. EI-MS m/z: [M/2+H]⁺ 799.81.

### Preparation of Intermediate Compound 269

To a solution of Intermediate Compound 268 (101 mg, 0.06 mmol) in methanol (1 mL) was added lithium hydroxide monohydrate (13 mg, 0.32 mmol) in distilled water (0.75 mL) at −50° C. under nitrogen. After stirred at 0° C. for 2 hours, the reaction mixture was adjusted to pH 4-5 with acetic acid. the reaction mixture was concentrated under reduced pressure to afford Intermediate Compound 269 (crude), which was used without further purification. EI-MS m/z: [M/2+H]⁺730.34.

### Preparation of Compound 270

To a solution of Intermediate Compound 269 (0.06 mmol, crude) in dichloromethane (2 mL) was added trifluoroacetic acid (0.6 mL) at 0° C. under nitrogen. After stirred at room temperature for 2 hours, the reaction solution was concentrated under reduced pressure. The resulting residue was purified by HPLC to afford Compound 270 (53.8 mg, 54%; 2 steps). EI-MS m/z: [M+H]⁺ 1344.70, [M/2+H]⁺673.21.

### Example 41: Preparation of Compound 272

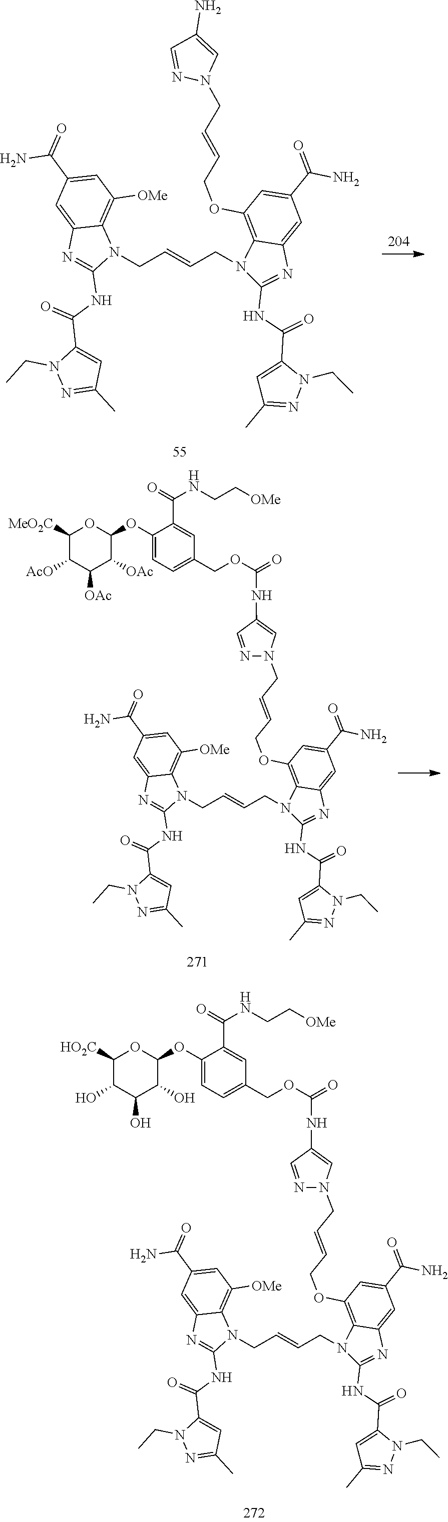

### Preparation of Intermediate Compound 271

To a solution of Intermediate Compound 55 (39 mg, 0.03 mmol) in N,N-dimethylformamide (2 mL) was added Intermediate Compound 204 (25 mg, 0.03 mmol), N,N′-diisopropylethylamine (0.03 mL, 0.16 mmol) and HOAt (1-Hydroxy-7-azabenzotriazole, 0.4 mg, 0.003 mmol). After stirred at room temperature for 3 hours, the reaction mixture was concentrated under reduced pressure to afford Intermediate Compound 271 (crude), which was used without further purification. EI-MS m/z: [M+H]⁺ 1425.67.

### Preparation of Compound 272

To a solution of Intermediate Compound 271 (crude, 0.03 mmol) in methanol (0.5 mL) and tetrahydrofuran (0.5 mL) was added lithium hydroxide monohydrate (13.5 mg, 0.3 mmol) in distilled water (0.7 mL) at −45° C. under nitrogen. After stirred at 0° C. for 1 hours, the reaction mixture was adjusted to pH 4-5 with acetic acid. The reaction solution was concentrated under reduced pressure. The resulting residue was purified by HPLC to afford Compound 272 (14 mg).

¹H-NMR (400 MHz, DMSO-d₆) δ 9.51 (s, 1H), 8.29 (s, 1H), 7.95 (d, J=15.5 Hz, 2H), 7.83 (s, 1H), 7.64 (d, J=6.9 Hz, 2H), 7.48 (d, J=8.6 Hz, 1H), 7.36-7.28 (m, 4H), 7.24 (d, J=8.6 Hz, 1H), 6.51 (s, 2H), 5.91-5.88 (m, 1H), 5.79 (s, 2H), 5.69-5.64 (m, 1H), 5.14 (d, J=6.7 Hz, 1H), 5.05 (s, 2H), 4.89 (d, J=10.7 Hz, 3H), 4.54-4.49 (m, 6H), 3.96 (d, J=9.5 Hz, 1H), 3.70 (s, 3H), 2.09 (t, J=2.8 Hz, 6H), 1.25 (q, J=6.8 Hz, 6H). EI-MS m/z: [M+H]⁺ 1285.54.

### Example 42: Preparation of Compound 274

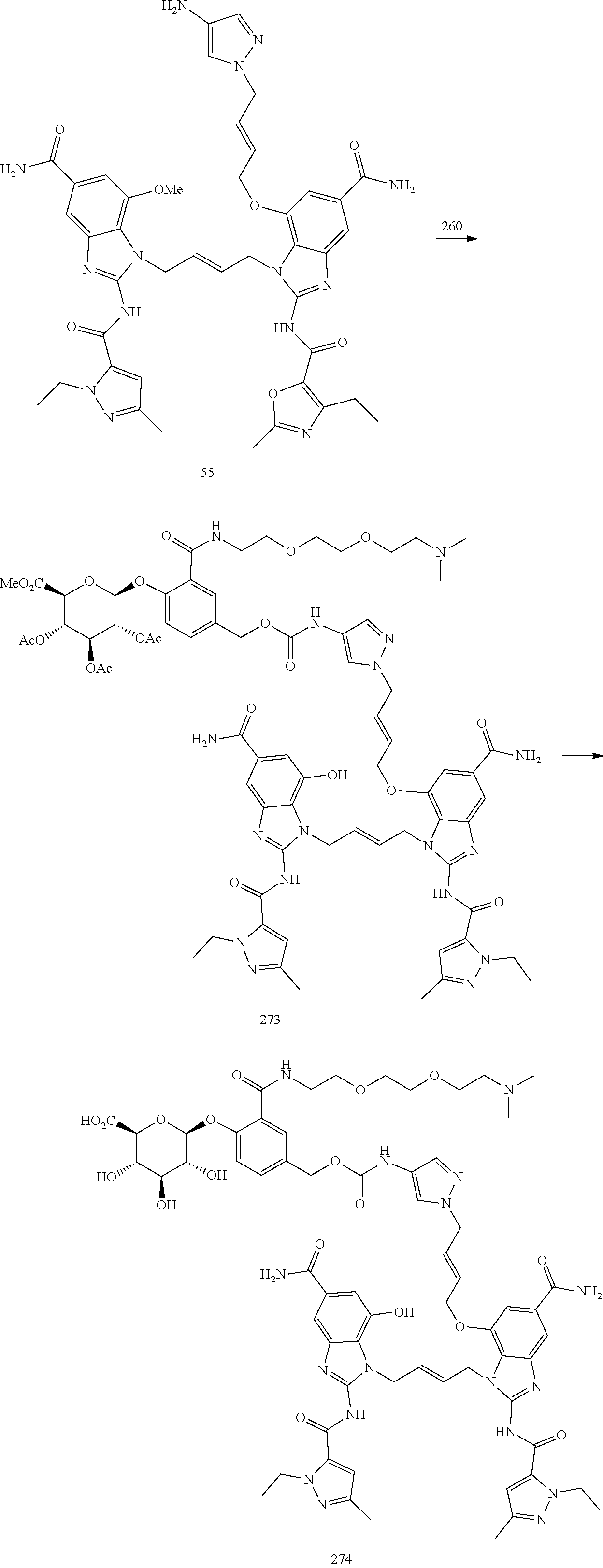

### Preparation of Intermediate Compound 273

To a solution of Intermediate Compound 55 (34 mg, 0.04 mmol) in N,N-dimethylformamide (2 mL) was added Intermediate Compound 260 (40 mg, 0.03 mmol), N,N-diisopropylethylamine (0.03 mL, 0.17 mmol) and HOAt (1-Hydroxy-7-azabenzotriazole, 0.9 mg, 0.007 mmol). After stirred at room temperature for 3 hours, the reaction mixture was concentrated under reduced pressure to afford Intermediate Compound 273 (crude), which was used without further purification. EI-MS m/z: [M+1/2]⁺ 764.28, [M+H]⁺ 1527.93.

### Preparation of Compound 274

To a solution of Intermediate Compound 273 (crude, 0.039 mmol) in methanol (2 mL) was added lithium hydroxide monohydrate (16 mg, 0.39 mmol) in distilled water (1 mL) at −50° C. under nitrogen. After stirred at 0° C. for 3 hours, the reaction mixture was adjusted to pH 4-5 with acetic acid. The reaction solution was concentrated under reduced pressure. The resulting residue was purified by HPLC to afford Compound 274 (10.6 mg, 19%). EI-MS m/z: [M+1/2]⁺694.16, [M+H]⁺ 1386.77.

### Example 43: Preparation of Compound 277

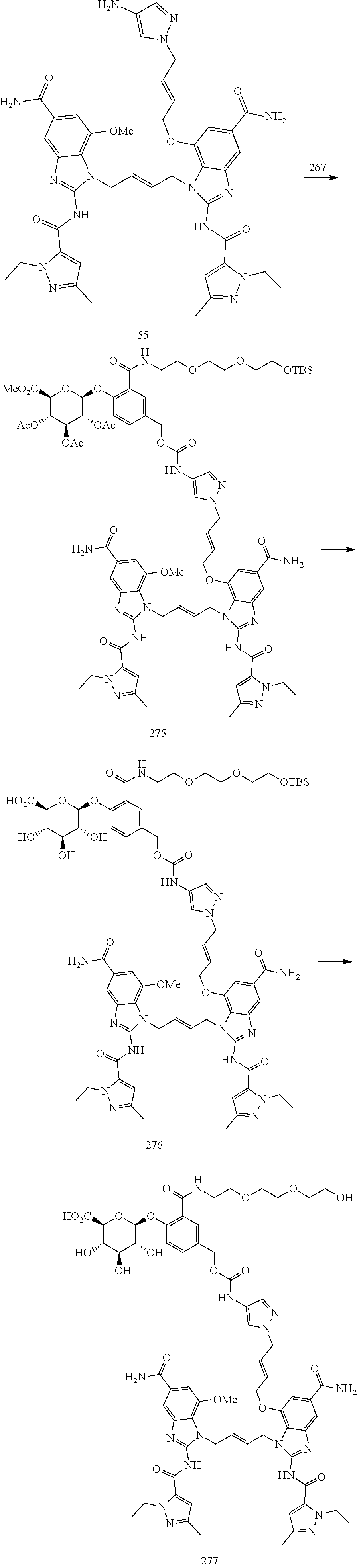

### Preparation of Intermediate Compound 275

To a solution of Intermediate Compound 55 (40 mg, 0.033 mmol) in N,N-dimethylformamide (1 mL) was added Intermediate Compound 267 (34.3 mg, 0.037 mmol), N,N′-diisopropylethylamine (0.03 mL, 0.167 mmol) and HOAt (1-Hydroxy-7-azabenzotriazole, 0.9 mg, 0.007 mmol). After stirred at room temperature for 4 hours, the reaction mixture was concentrated under reduced pressure to afford Intermediate Compound 275 (crude), which was used without further purification. EI-MS m/z: [M/2+H]⁺ 807.55.

### Preparation of Intermediate Compound 276

To a solution of Intermediate Compound 275 (53.7 mg, 0.033 mmol) in methanol (0.75 mL) and tetrahydrofuran (0.75 mL) was added lithium hydroxide monohydrate (14 mg, 0.33 mmol) in distilled water (1 mL) at −50° C. under nitrogen. After stirred at 0° C. for 4 hours, the reaction mixture was adjusted to pH 4-5 with acetic acid. The reaction solution was concentrated under reduced pressure to afford Intermediate Compound 276 (crude), which was used without further purification. EI-MS m/z: [M/2+H]⁺ 737.69.

### Preparation of Compound 277

To a solution of Intermediate Compound 276 (crude, 0.033 mmol) in dichloromethane (1 mL) was added trifluoroacetic acid (1 mL) at 0° C. under nitrogen. After stirred at room temperature for 2 hours, the reaction solution was concentrated under reduced pressure. The resulting residue was purified by HPLC to afford Compound 277 (13 mg, 29%; 3 steps). EI-MS m/z: [M+H]⁺ 1359.59, [M/2+H]⁺ 680.61.

### Example 44: Preparation of Compound 281

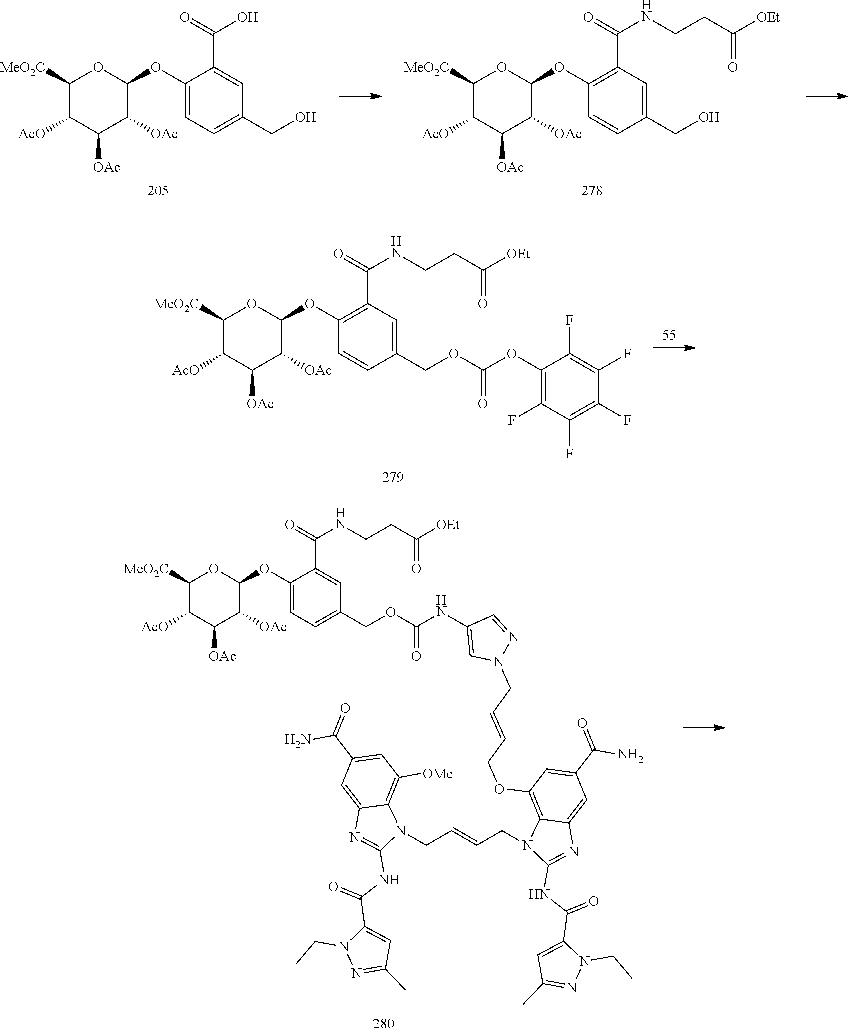

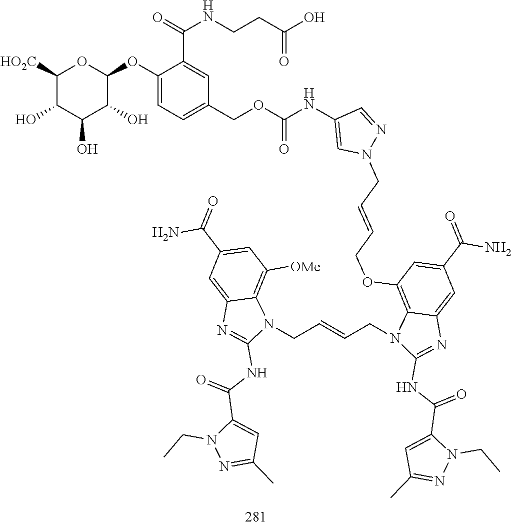 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCC(=O)O)c4)cn3)c21]

### Preparation of Intermediate Compound 278

To a solution of intermediate compound 205 (1 g, 2.06 mmol, compound 205 was prepared according to the method described in the International patent publication No. WO 2018/182341 A1) was dissolved in N,N-dimethylformamide (10 mL) was added β-Alanine ethyl ester hydrochloride (380 mg, 2.48 mmol), 2-(1H-Benzotriazole-1-yl)-1,1,3,3-tetramethylaminium tetrafluoroborate (HBTU, 1.02 g, 2.68 mmol) and N,N′-diisopropylethylamine (0.72 mL, 4.13 mmol) at −0° C. After raised to room temperature and stirred under nitrogen for 18 hours. The reaction mixture was diluted with ethyl acetate (50 mL) and washed with saturated aqueous sodium hydrogen carbonate (50 mL) and distilled water (50 mL). The organic layer was dried over anhydrous sodium sulfate and filtered. The filtrate was concentrated under reduced pressure. The resulting residue was purified by column chromatography to afford Intermediate Compound 278 (398 mg, 32%).

¹H-NMR (400 MHz, CDCl₃) δ 8.02 (d, J=2.6 Hz, 1H), 7.71 (s, 1H), 7.48 (d, J=8.5 Hz, 1H), 7.26 (s, 2H), 7.06-6.99 (m, 1H), 5.40 (dt, J=18.9, 7.7 Hz, 3H), 5.29 (d, J=7.3 Hz, 1H), 4.68 (d, J=2.6 Hz, 2H), 4.26-4.14 (m, 3H), 3.79 (d, J=6.9 Hz, 1H), 3.74 (d, J=2.6 Hz, 3H), 3.69-3.60 (m, 1H), 2.67 (d, J=7.0 Hz, 2H), 2.09-2.03 (m, 9H), 1.29 (td, J=7.2, 2.5 Hz, 3H).

### Preparation of Intermediate Compound 279

To a solution of Intermediate Compound 278 (398 mg, 0.68 mmol) in dichloromethane (4 mL) was added bis(pentafluorophenyl)carbonate (323 mg, 0.82 mmol) and N,N-diisopropylethylamine (0.36 ml, 2.04 mmol) at −0° C. After raised to room temperature and stirred under nitrogen for 14 hours. The reaction mixture was diluted with dichloromethane (15 mL) and washed with distilled water (15 mL×2). The organic layer was dried over anhydrous sodium sulfate and filtered. The filtrate was concentrated under reduced pressure to afford Intermediate Compound 279 (280 mg, 51%). EI-MS m/z: [M+H]⁺ 793.88.

### Preparation of Intermediate Compound 280

To a solution of Intermediate Compound 55 (300 mg, 0.25 mmol) in N,N-dimethylformamide (5 mL) was added compound 279 (218 mg, 0.28 mmol) and N,N′-diisopropylethylamine (0.22 mL, 0.13 mmol). After stirred at room temperature for 4 hours, the reaction solution was concentrated under reduced pressure. The resulting residue was purified by column chromatography to afford Intermediate Compound 280 (370 mg, crude). EI-MS m/z: [M+H]⁺ 1467.76.

### Preparation of Compound 281

To a solution of Intermediate Compound 280 (36 mg, 0.025 mmol) in methanol (1 mL) and tetrahydrofuran (1 mL) was added lithium hydroxide monohydrate (5.24 mg, 0.13 mmol) in distilled water (1 mL) at −50° C. under nitrogen. After stirred at 0° C. for 4 hours, the reaction mixture was adjusted to pH 4-5 with acetic acid. The reaction solution was concentrated under reduced pressure. The resulting residue was purified by HPLC to afford Compound 281 (13 mg). EI-MS m/z: [M+H]⁺ 1299.88.

### Example 45: Preparation of Intermediate Compound 285

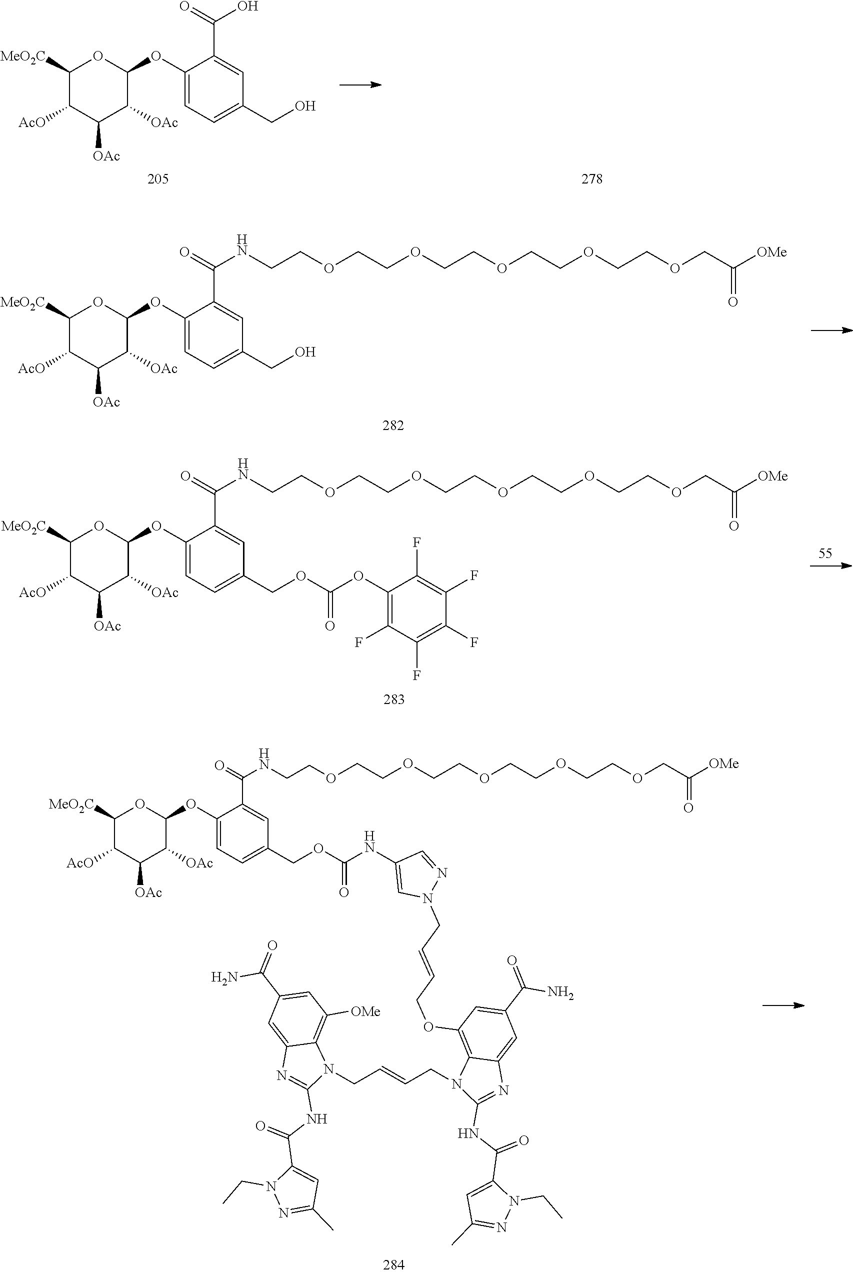

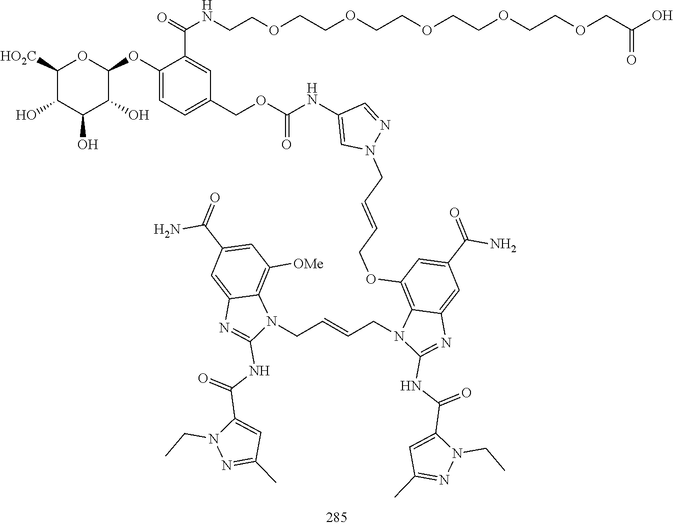 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCOCCOCCOCC(=O)O)c4)cn3)c21]

### Preparation of Intermediate Compound 282

To a solution of intermediate compound 205 (1 g, 2.06 mmol, compound 205 was prepared according to the method described in the International patent publication No. WO 2018/182341 A1) was dissolved in dichloromethane (3 mL) was added methyl 17-amino-3,6,9,12,15-pentaoxaheptadecanoate (766 mg, 2.48 mmol), N-methylmorpholine (0.57 mL, 5.16 mmol), N-Ethyl-N-(3-dimethylaminopropyl)carbodiimide hydrochloride (EDC HCl, 415 mg, 2.17 mmol) and 1-Hydroxybenzotriazole (306 mg, 2.27 mmol) at −0° C. After raised to room temperature and stirred under nitrogen for 18 hours. The reaction mixture was diluted with ethyl acetate (50 mL) and washed with saturated aqueous sodium hydrogen carbonate (50 mL) and distilled water (50 mL). The organic layer was dried over anhydrous sodium sulfate and filtered. The filtrate was concentrated under reduced pressure. The resulting residue was purified by column chromatography to afford Intermediate Compound 282 (673 mg, 42%). EI-MS m/z: [M+H]⁺ 775.97.

### Preparation of Intermediate Compound 283

To a solution of Intermediate Compound 282 (670 mg, 0.86 mmol) in dichloromethane (5 mL) was added bis(pentafluorophenyl)carbonate (408 mg, 1.04 mmol) and N,N-diisopropylethylamine (0.45 ml, 2.59 mmol) at −0° C. After raised to room temperature and stirred under nitrogen for 18 hours. The reaction mixture was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 283 (280 mg, 51%). EI-MS m/z: [M+H]⁺ 986.17.

### Preparation of Intermediate Compound 284

To a solution of Intermediate Compound 283 (300 mg, 0.25 mmol) in N,N-dimethylformamide (1.5 mL) were added N,N′-diisopropylethylamine (0.05 mL, 0.28 mmol) and Intermediate Compound 55 (60 mg, 0.06 mmol) at 0° C. under nitrogen. After stirred at room temperature for 4 hours, the reaction solution was concentrated under reduced pressure. The resulting residue was purified by column chromatography to afford Intermediate Compound 284 (91.9 mg, crude). EI-MS m/z: [1/2M+H]⁺ 830.331.

### Preparation of Compound 285

To a solution of Intermediate Compound 284 (92 mg, 0.06 mmol) in tetrahydrofuran (1 mL) and methanol (1 mL) was added lithium hydroxide monohydrate (23 mg, 0.55 mmol) in distilled water (1 mL) at −70° C. under nitrogen. After stirred at 0° C. for 4 hours, the reaction mixture was adjusted to pH 4-5 with acetic acid. The reaction solution was concentrated under reduced pressure. The resulting residue was purified by HPLC to afford Compound 285 (35 mg, 42%). EI-MS m/z: [M+H]⁺ 1505.876.

### Example 46: Preparation of Compound 291

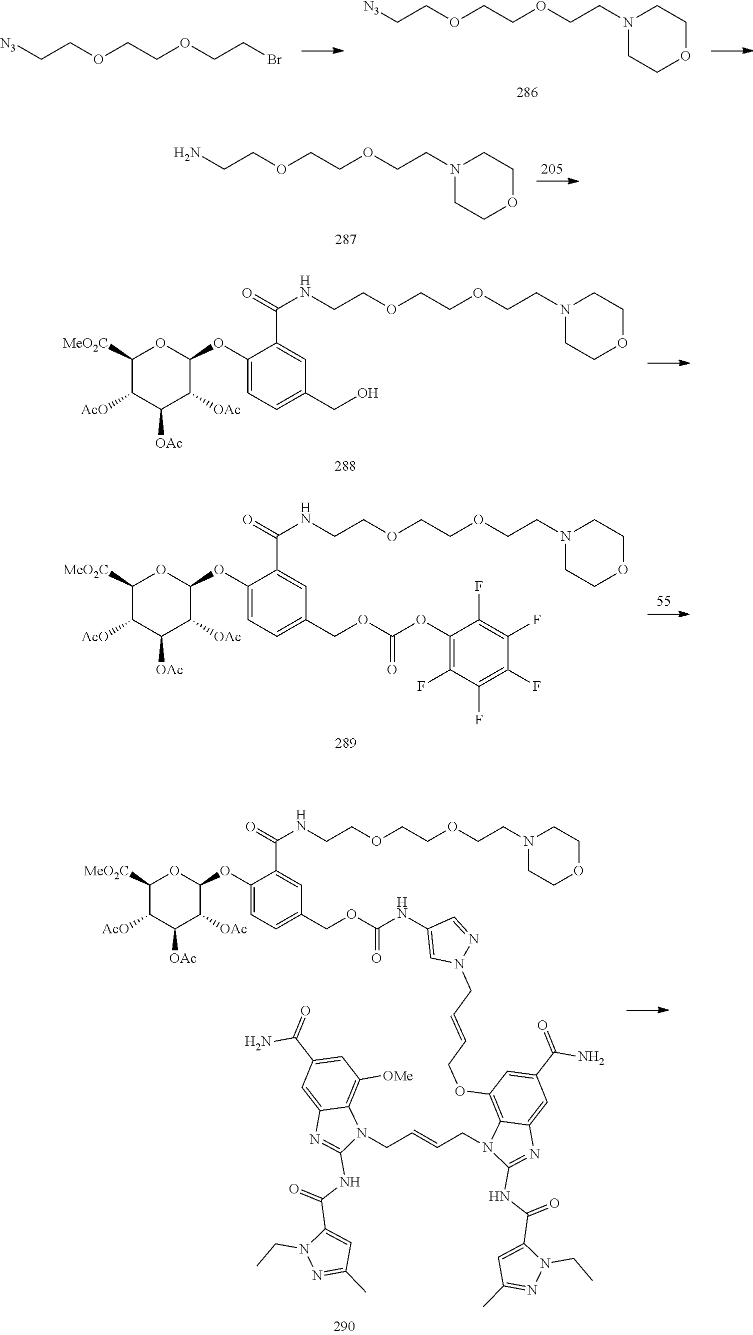

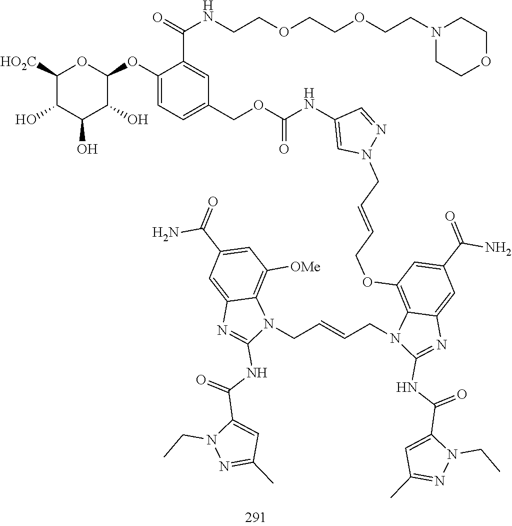 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN5CCOCC5)c4)cn3)c21]

### Preparation of Intermediate Compound 286

1-Azido-2-(2-(2-bromoethoxy)ethoxy)ethane (1.5 g, 6.3 mmol) was added morpholine (1.63 mL, 18.9 mmol). After stirred at 50° C. for 5 hours, the reaction solution was concentrated under reduced pressure. The resulting residue purified by column chromatography to afford Intermediate Compound 286 (793 mg, 52%).

### Preparation of Intermediate Compound 287

To a solution of Intermediate Compound 286 (793 mg, 3.25 mmol) in tetrahydrofuran (3 mL) were added triphenylphosphine (937 mg, 3.57 mmol) and distilled water (1 mL) at 0° C. under nitrogen. The reaction mixture was refluxed for 15 hours. After concentrated under reduced pressure, the resulting residue purified by column chromatography to afford Intermediate Compound 287 (607 mg, 86%).

¹H-NMR (400 MHz, CDCl₃) δ 3.72 (t, J=4.6 Hz, 4H), 3.63 (d, J=2.5 Hz, 5H), 3.55 (d, J=5.0 Hz, 1H), 2.98-2.88 (m, 4H), 2.60 (td, J=5.9, 2.8 Hz, 2H), 2.51 (t, J=4.8 Hz, 4H).

### Preparation of Intermediate Compound 288

To a solution of Intermediate Compound 205 (607 mg, 2.78 mmol, Intermediate Compound 205 was prepared by the method described in the International patent publication No. WO 2018/182341 A1) in dichloromethane (15 mL) were added N-(3-dimethylaminopropyl)-N′-ethylcarbodiimide hydrochloride (EDC HCl, 560 mg, 2.92 mmol), 1-hydroxybenzotriazole (HOBt, 413 mg, 3.06 mmol), Intermediate Compound 287 (1.62 g, 3.34 mmol) and N-methylmorpholine (0.76 mL, 6.95 mmol) at 0° C. under nitrogen. After stirred at room temperature for 15 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 288 (1.07 mg, 56%). EI-MS m/z: [M+H]⁺ 685.98.

### Preparation of Intermediate Compound 289

To a solution of Intermediate Compound 288 (100 mg, 0.15 mmol) in dichloromethane (1 mL) were added bis(pentafluorophenyl)carbonate (73 mg, 0.18 mmol) and N,N′-diisopropylethylamine (0.08 mL, 0.44 mmol) at 0° C. under nitrogen. After stirred at room temperature for 6 hours, the reaction solution was diluted with dichloromethane (15 mL) and washed with distilled water (15 mL×2). The organic layer was dried over anhydrous sodium sulfate and filtered. The filtrate was concentrated under reduced pressure to afford Intermediate Compound 289 (120 mg), which was used without further purification. EI-MS m/z: [M+H]⁺ 895.98.

### Preparation of Intermediate Compound 290

To a solution of Intermediate Compound 55 (40 mg, 0.05 mmol) in N,N-dimethylformamide (0.05 mL) were added N,N′-diisopropylethylamine (0.04 mL, 0.22 mmol) and Intermediate Compound 289 (54 mg, 0.05 mmol) at 0° C. under nitrogen. After stirred at room temperature for 17 hours, the reaction solution was concentrated under reduced pressure to afford Intermediate Compound 290 (crude), which was used without further purification. EI-MS m/z: [M+H]⁺ 1568.97.

### Preparation of Compound 291

To a solution of Intermediate Compound 290 (70 mg, 0.05 mmol) in tetrahydrofuran (1 mL) and methanol (1 mL) was added lithium hydroxide monohydrate (18.76 mg, 0.45 mmol) in distilled water (1 mL) at −70° C. under nitrogen. After stirred at 0° C. for 3 hours, the reaction mixture was adjusted to pH 4-5 with acetic acid and concentrated under reduced pressure. The resulting residue was purified by HPLC to afford Compound 291 (42 mg, 53%). EI-MS m/z: [M+H]⁺ 1429.19.

### Example 47: Preparation of Compound 299

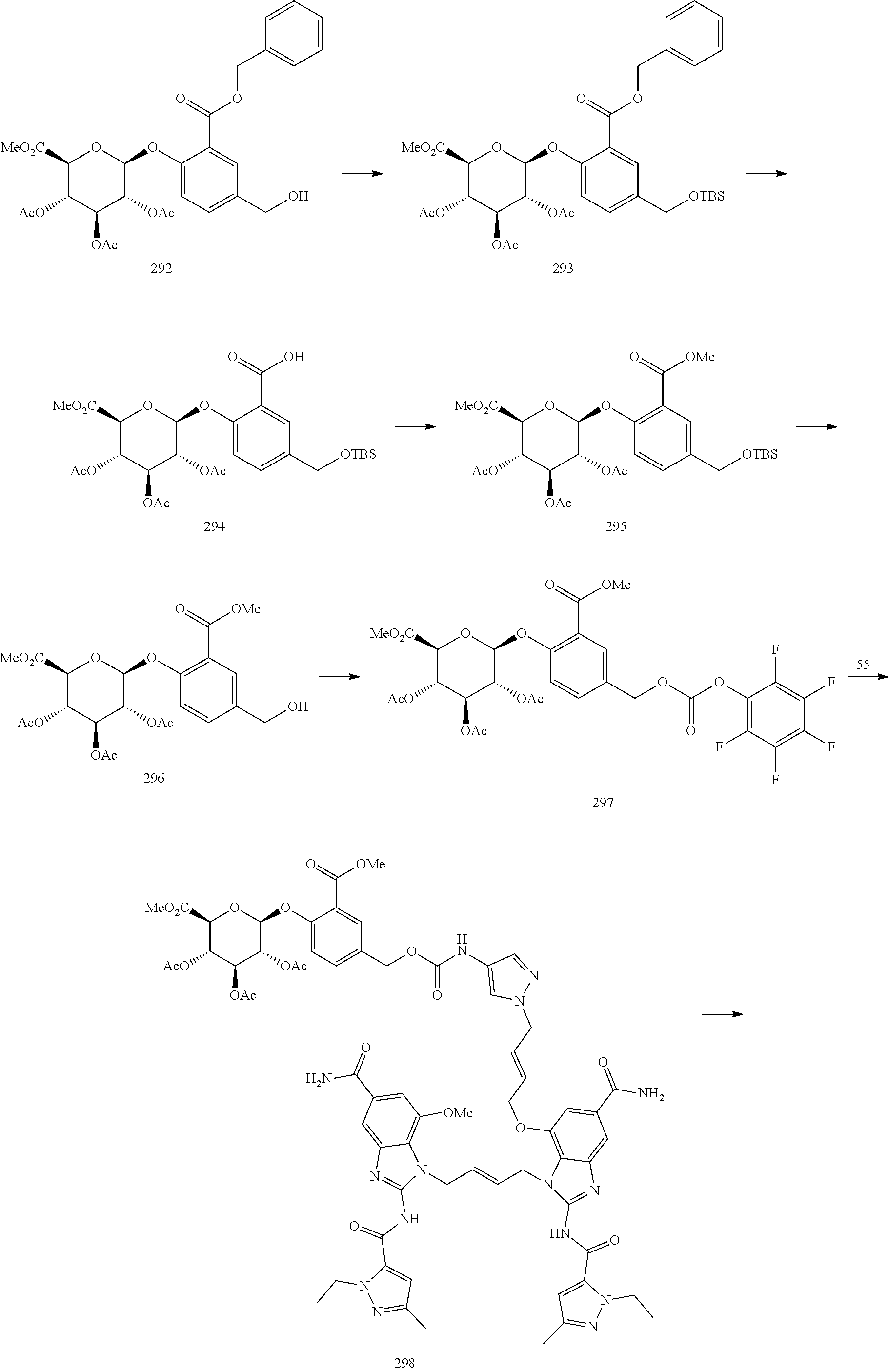

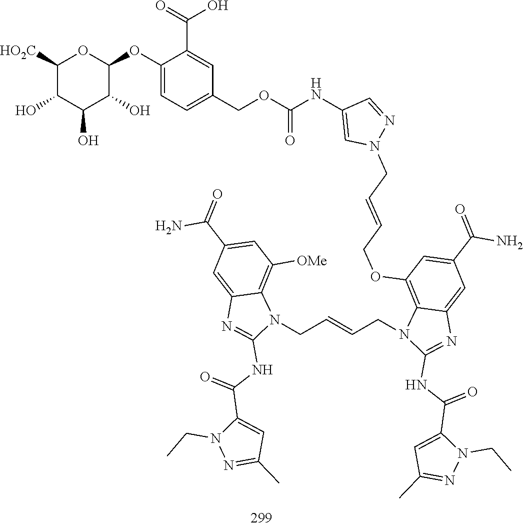 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)O)c4)cn3)c21]

### Preparation of Intermediate Compound 293

To a solution of Intermediate Compound 292 (10 g, 17.41 mmol, Intermediate Compound 292 was prepared by the method described in the International patent publication No. WO 2018/182341 A1) in dichloromethane (15 mL) were added imidazole (2.4 g, 34.81 mmol) and t-butyldimethylsilyl chloride (3.94 g, 26.11 mmol) under nitrogen. After stirred at room temperature for 2 hours, the reaction solution was diluted with dichloromethane (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 293 (10.4 g, 87%).

¹H-NMR (400 MHz, CDCl₃) δ 7.70 (s, 1H), 7.47-7.30 (m, 6H), 7.15 (d, J=8.7 Hz, 1H), 5.31 (d, J=17.6 Hz, 5H), 5.14 (d, J=5.6 Hz, 1H), 4.68 (s, 2H), 4.18-4.11 (m, 1H), 3.73 (d, J=2.9 Hz, 3H), 2.05 (s, 9H), 0.92 (d, J=3.0 Hz, 9H), 0.08 (d, J=2.9 Hz, 6H).

### Preparation of Intermediate Compound 294

To a solution of Intermediate Compound 293 (10 g, 15.1 mmol) in tetrahydrofuran (110 mL) and methanol (110 mL) were added 5% palladium/charcoal (520 mg) and distilled water (10 mL) under hydrogen. After stirred at room temperature for 2 hours, the reaction solution was filtered through Celite and concentrated under reduced pressure. The resulting residue was purified by column chromatography to afford Intermediate Compound 294 (7.9 g, 87%).

¹H-NMR (400 MHz, CDCl₃) δ 8.01 (d, J=2.5 Hz, 1H), 7.58 (d, J=8.2 Hz, 1H), 7.21 (d, J=8.5 Hz, 1H), 5.46-5.26 (m, 4H), 4.72 (s, 2H), 4.30 (d, J=8.2 Hz, 1H), 3.69 (d, J=1.9 Hz, 3H), 2.14-1.99 (m, 9H), 0.94 (d, J=2.0 Hz, 9H), 0.10 (s, 6H).

### Preparation of Intermediate Compound 295

To a solution of Intermediate Compound 294 (500 mg, 0.84 mmol) in N,N-dimethylformamide (3 mL) were added iodomethane (0.3 mL, 4.18 mmol) and potassium carbonate (144 mg, 1.04 mmol) under nitrogen. After stirred at room temperature for 4 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 295 (510 g, 99%). EI-MS m/z: [M+Na]⁺634.976.

### Preparation of Intermediate Compound 296

To a solution of Intermediate Compound 295 (510 mg, 0.83 mmol) in methanol (3 mL) was added camphorsulfonic acid (39 mg, 0.17 mmol) at 0° C. under nitrogen. After stirred at 0° C. for 2 hours, the reaction solution was diluted with ethyl acetate (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 296 (360 g, 87%). EI-MS m/z: [M+Na]⁺521.024.

¹H-NMR (400 MHz, CDCl₃) δ 7.76 (s, 1H), 7.47 (d, J=8.4 Hz, 1H), 7.26 (s, 2H), 7.15 (dd, J=8.9, 2.5 Hz, 1H), 5.40-5.30 (m, 3H), 5.15 (d, J=5.9 Hz, 1H), 4.67 (d, J=4.8 Hz, 2H), 4.23-4.15 (m, 1H), 3.86 (d, J=2.7 Hz, 3H), 3.74 (d, J=2.8 Hz, 3H), 2.10-2.02 (m, 9H).

### Preparation of Intermediate Compound 297

To a solution of Intermediate Compound 296 (185 mg, 0.24 mmol) in dichloromethane (5 mL) were added bis(pentafluorophenyl)carbonate (342 mg, 0.87 mmol) and N,N-diisopropylethylamine (0.38 ml, 2.17 mmol) at 0° C. under nitrogen. After stirred at room temperature for 14 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 297 (410 mg, 80%). EI-MS m/z: [M+Na]⁺ 730.794.

### Preparation of Intermediate Compound 298

To a solution of Intermediate Compound 297 (50 mg, 0.04 mmol) in N,N-dimethylformamide (0.05 mL) were added N,N′-diisopropylethylamine (0.04 mL, 0.21 mmol) and Intermediate Compound 55 (35 mg, 0.05 mmol) under nitrogen. After stirred at room temperature for 15 hours, the reaction solution was concentrated under reduced pressure to afford Intermediate Compound 298 (125 mg, crude), which was used without further purification. EI-MS m/z: [M+H]⁺ 1832.77.

### Preparation of Compound 299

To a solution of Intermediate Compound 298 (58 mg, 0.04 mmol) in tetrahydrofuran (1 mL) and methanol (1 mL) was added lithium hydroxide monohydrate (17.48 mg, 0.42 mmol) in distilled water (1 mL) at −70° C. under nitrogen. After stirred at 0° C. for 3 hours, the reaction mixture was adjusted to pH 4-5 with acetic acid and concentrated under reduced pressure. The resulting residue was purified by HPLC to afford Compound 299 (50 mg, 82%). EI-MS m/z: [M/2+H]⁺614.87.

### Example 48: Preparation of Compound 302

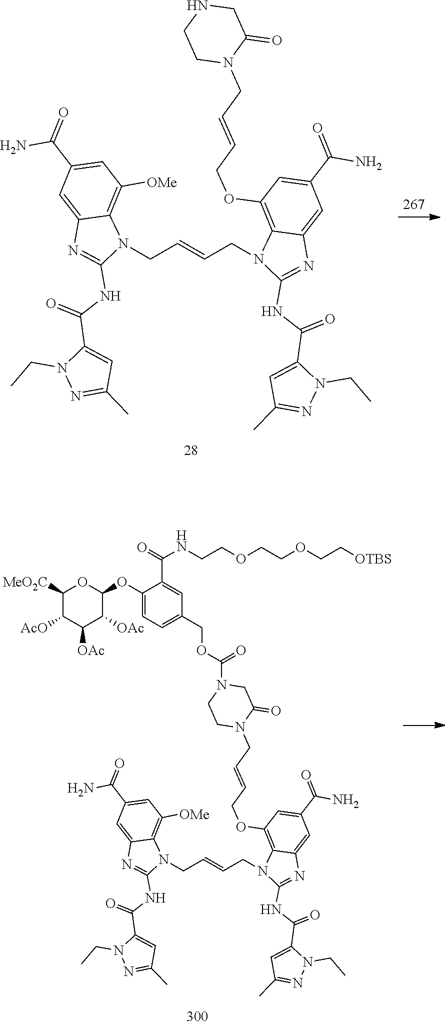 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCNCC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)OC)[C@@H](OC(C)=O)[C@H](OC(C)=O)[C@H]5OC(C)=O)c(C(=O)NCCOCCOCCO[Si](C)(C)C(C)(C)C)c4)CC3=O)c21]

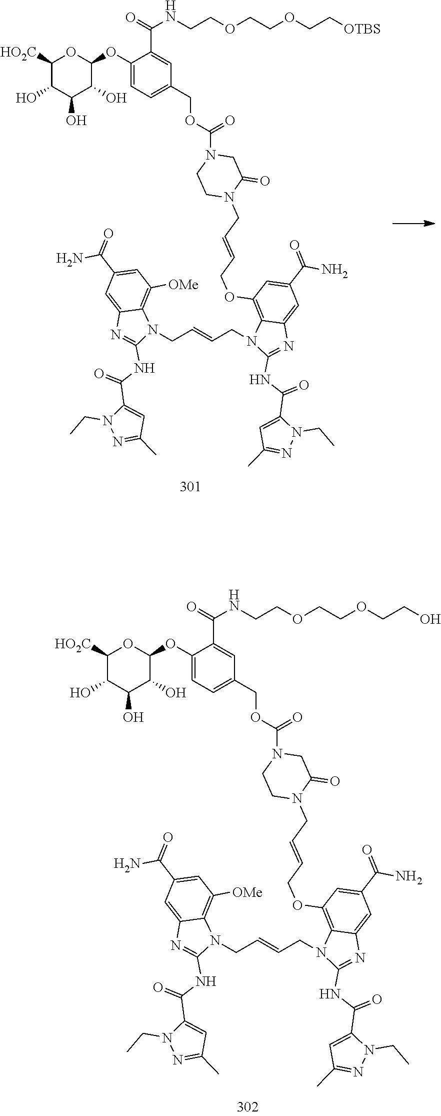 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)O)[C@@H](O)[C@H](O)[C@H]5O)c(C(=O)NCCOCCOCCO[Si](C)(C)C(C)(C)C)c4)CC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)O)[C@@H](O)[C@H](O)[C@H]5O)c(C(=O)NCCOCCOCCO)c4)CC3=O)c21]

### Preparation of Intermediate Compound 300

To a solution of Intermediate Compound 28 (30 mg, 0.02 mmol) in N,N-dimethylformamide (1 mL) were added Intermediate Compound 267 (25.5 mg, 0.03 mmol), N,N′-diisopropylethylamine (0.02 mL, 0.12 mmol) and 1-hydroxy-7-azabenzotriazole (HOAt, 2 mg, 0.02 mmol) under nitrogen. After stirred at room temperature for 2 hours, the reaction solution was concentrated under reduced pressure to afford Intermediate Compound 300 (crude), which was used without further purification. EI-MS m/z: [M/2+H]⁺ 759.25.

### Preparation of Intermediate Compound 301

To a solution of Intermediate Compound 300 (36.5 mg, 0.02 mmol) in tetrahydrofuran (0.5 mL) and methanol (0.5 mL) was added lithium hydroxide monohydrate (5.1 mg, 0.12 mmol) in distilled water (0.5 mL) at −70° C. under nitrogen. After stirred at 0° C. for 2 hours, the reaction mixture was adjusted to pH 4-5 with acetic acid and concentrated under reduced pressure to afford Intermediate Compound 301 (crude), which was used without further purification. EI-MS m/z: [M/2+H]⁺ 746.27.

### Preparation of Compound 302

To a solution of Intermediate Compound 301 (36.5 mg) in dichloromethane (1 mL) was added trifluoroacetic acid (0.25 mL) at 0° C. under nitrogen. After stirred at room temperature for 40 minutes, the reaction solution was concentrated under reduced pressure. The resulting residue was purified by HPLC to afford Compound 302 (24.8 mg, 63%). EI-MS m/z: [M+H]⁺ 1376.63, [M/2+H]⁺ 689.09.

### Example 49: Preparation of Compound 307

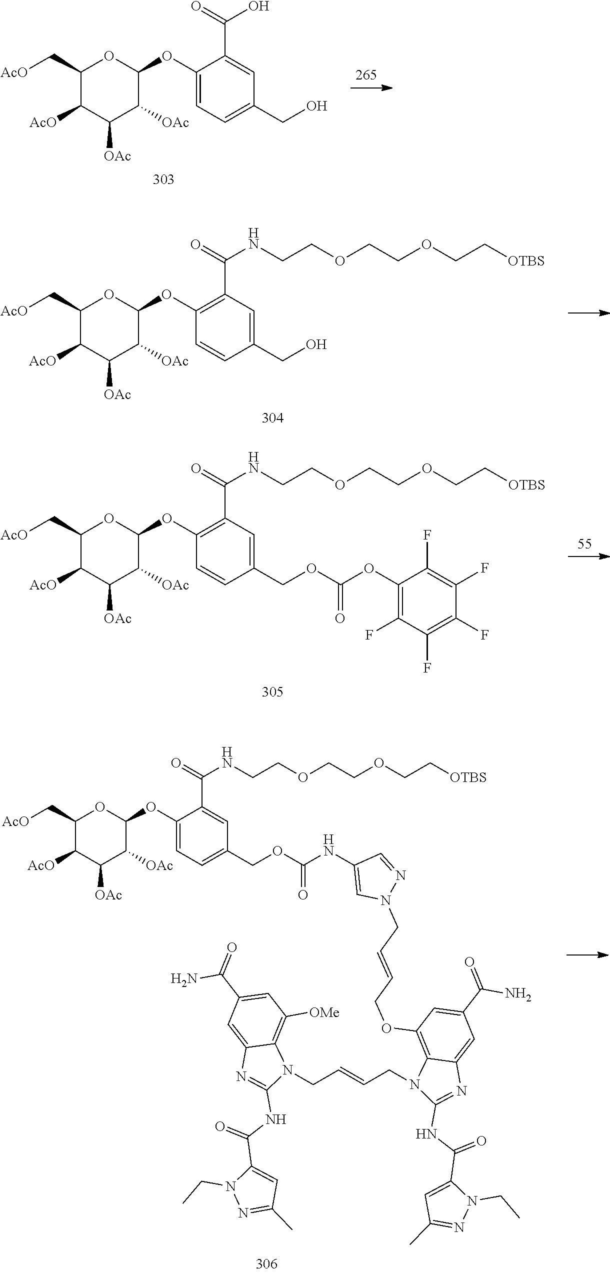

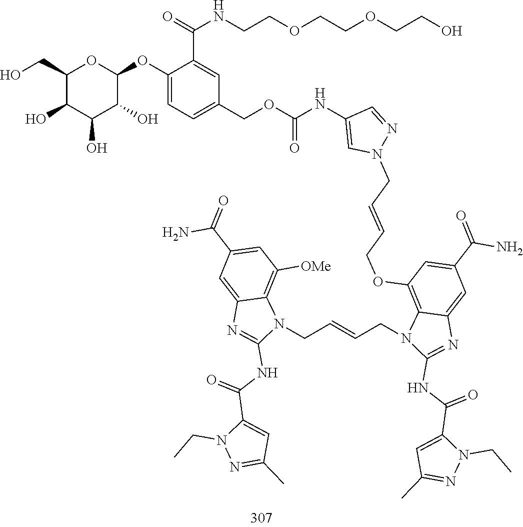 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](CO)[C@H](O)[C@H](O)[C@H]5O)c(C(=O)NCCOCCOCCO)c4)cn3)c21]

### Preparation of Intermediate Compound 304

To a solution of Intermediate Compound 303 (300 mg, 0.60 mmol, Intermediate Compound 303 was prepared by the method described in the International patent publication No. WO 2018/182341 A1) in N,N-dimethylformamide (3 mL) were added N,N,N′,N′-tetramethyl-O-(1H-benzotriazol-1-yl)uronium hexafluorophosphate (HBTU, 297 mg, 0.78 mmol), Intermediate Compound 265 (190 mg, 0.72 mmol) and N,N′-diisopropylethylamine (0.23 mL, 2.16 mmol) at 0° C. under nitrogen. After stirred at room temperature for 3 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 304 (379 mg, 85%).

¹H-NMR (400 MHz, CDCl₃) δ 8.04 (s, 1H), 7.46 (d, J=8.5 Hz, 1H), 7.38 (s, 1H), 7.06 (d, J=8.5 Hz, 1H), 5.58-5.46 (m, 2H), 5.16 (d, J=6.6 Hz, 2H), 4.68 (s, 2H), 4.25-4.09 (m, 3H), 3.74 (d, J=6.2 Hz, 3H), 3.67 (d, J=10.4 Hz, 6H), 3.54 (t, J=5.8 Hz, 3H), 2.22 (s, 3H), 2.06 (d, J=2.7 Hz, 6H), 2.02 (s, 3H), 0.89 (d, J=2.0 Hz, 9H), 0.07 (s, 6H). EI-MS m/z: [M/2+H]⁺744.19.

### Preparation of Intermediate Compound 305

To a solution of Intermediate Compound 304 (379 mg, 0.51 mmol) in dichloromethane (5 mL) were added bis(pentafluorophenyl)carbonate (241 mg, 0.61 mmol) and N,N-diisopropylethylamine (0.27 ml, 1.53 mmol) at 0° C. under nitrogen. After stirred at room temperature for 3 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 305 (424 mg, 87%). EI-MS m/z: [M+H]⁺ 954.08.

### Preparation of Intermediate Compound 306

To a solution of Intermediate Compound 55 (30 mg, 0.03 mmol) in N,N-dimethylformamide (1 mL) were added Intermediate Compound 305 (26.2 mg, 0.03 mmol), N,N′-diisopropylethylamine (0.02 mL, 0.13 mmol) and 1-hydroxy-7-azabenzotriazole (HOAt, 0.7 mg, 0.005 mmol) at 0° C. under nitrogen. After stirred at room temperature for 2 hours, the reaction solution was concentrated under reduced pressure to afford Intermediate Compound 306 (crude), which was used without further purification. EI-MS m/z: [M/2+H]⁺814.51.

### Preparation of Compound 307

To a solution of Intermediate Compound 306 (40 mg, 0.02 mmol) in tetrahydrofuran (0.3 mL) and methanol (0.3 mL) was added lithium hydroxide monohydrate (10.3 mg, 0.25 mmol) in distilled water (0.3 mL) at −70° C. under nitrogen. After stirred at 0° C. for 2 hours, the reaction mixture was adjusted to pH 4-5 with acetic acid and concentrated under reduced pressure. The reaction mixture in dichloromethane (2 mL) was added trifluoroacetic acid (0.4 mL) at 0° C. under nitrogen. After stirred at room temperature for 2 hours, the reaction solution was concentrated under reduced pressure. The resulting residue was purified by HPLC to afford Compound 307 (24.4 mg, 63%). EI-MS m/z: [M+H]⁺ 1346.20, [M/2+H]⁺673.38.

### Example 50: Preparation of Compound 309

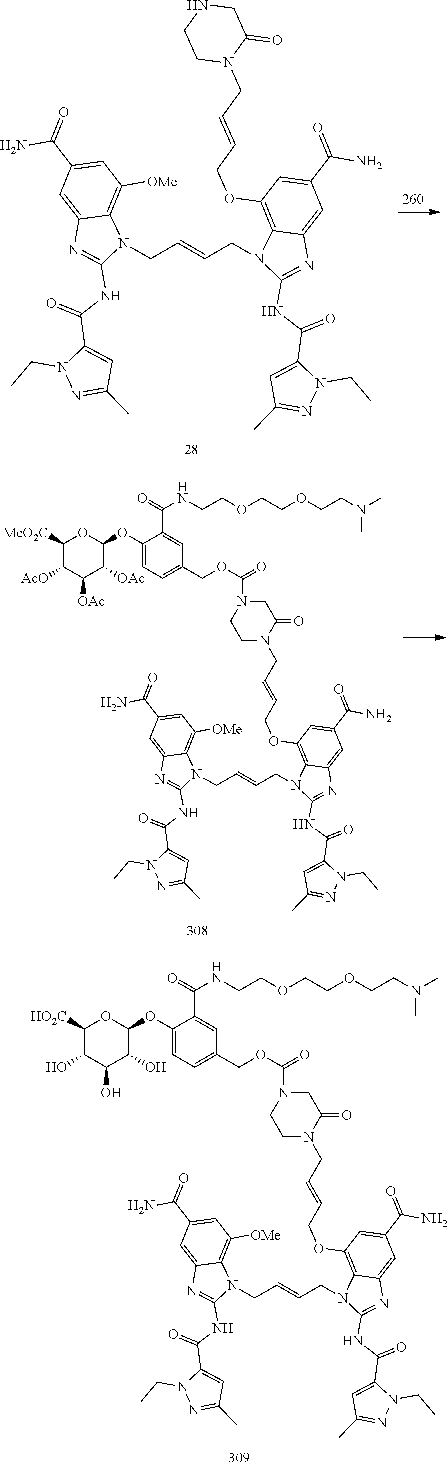

### Preparation of Intermediate Compound 308

To a solution of Intermediate Compound 28 (59 mg, 0.049 mmol) in N,N-dimethylformamide (2 mL) were added Intermediate Compound 260 (62 mg, 0.073 mmol), N,N′-diisopropylethylamine (0.04 mL, 0.24 mmol) and 1-hydroxy-7-azabenzotriazole (HOAt, 1.3 mg, 0.009 mmol) at 0° C. under nitrogen. After stirred at room temperature for 17 hours, the reaction solution was concentrated under reduced pressure. The resulting residue was purified by column chromatography to afford Intermediate Compound 308 (34 mg, 45%). EI-MS m/z: [M/2+H]⁺ 772.64.

### Preparation of Compound 309

To a solution of Intermediate Compound 308 (30 mg, 0.019 mmol) in tetrahydrofuran (0.5 mL) and methanol (0.5 mL) was added lithium hydroxide monohydrate (8.1 mg, 0.19 mmol) in distilled water (0.3 mL) at −70° C. under nitrogen. After stirred at 0° C. for 2 hours, the reaction mixture was adjusted to pH 4-5 with acetic acid and concentrated under reduced pressure. The resulting residue was purified by HPLC to afford Compound 309 (14 mg, 51%). EI-MS m/z: [M/2+H]⁺702.56.

### Preparation Example 6: Preparation of Intermediate Compound 310

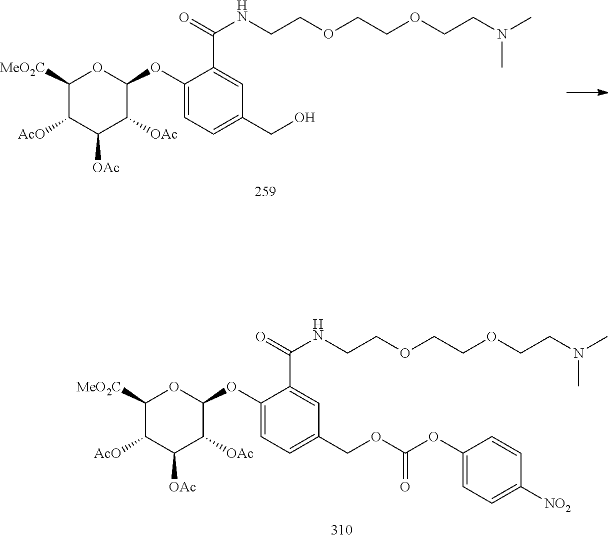 [SMILES: COC(=O)[C@H]1O[C@@H](Oc2ccc(CO)cc2C(=O)NCCOCCOCCN(C)C)[C@H](OC(C)=O)[C@@H](OC(C)=O)[C@@H]1OC(C)=O ; COC(=O)[C@H]1O[C@@H](Oc2ccc(COC(=O)Oc3ccc([N+](=O)[O-])cc3)cc2C(=O)NCCOCCOCCN(C)C)[C@H](OC(C)=O)[C@@H](OC(C)=O)[C@@H]1OC(C)=O]

### Preparation of Intermediate Compound 310

To a solution of Intermediate Compound 259 (300 mg, 0.47 mmol) in dichloromethane (60 mL) were added triethylamine (0.13 ml, 0.93 mmol) and 4-nitrophenyl chloroformate (122 mg, 0.61 mmol) at 0° C. under nitrogen. After stirred at room temperature for 4 hours, the reaction solution was diluted with dichloromethane (50 mL) and washed with saturated aqueous sodium bicarbonate solution (50 mL) and distilled water (50 mL). The organic layer was dried over anhydrous sodium sulfate and filtered. The filtrate was concentrated under reduced pressure to afford Intermediate Compound 310 (342 mg, Crude), which was used without further purification. EI-MS m/z: [M+H]⁺ 808.22.

### Example 51: Preparation of Compound 313

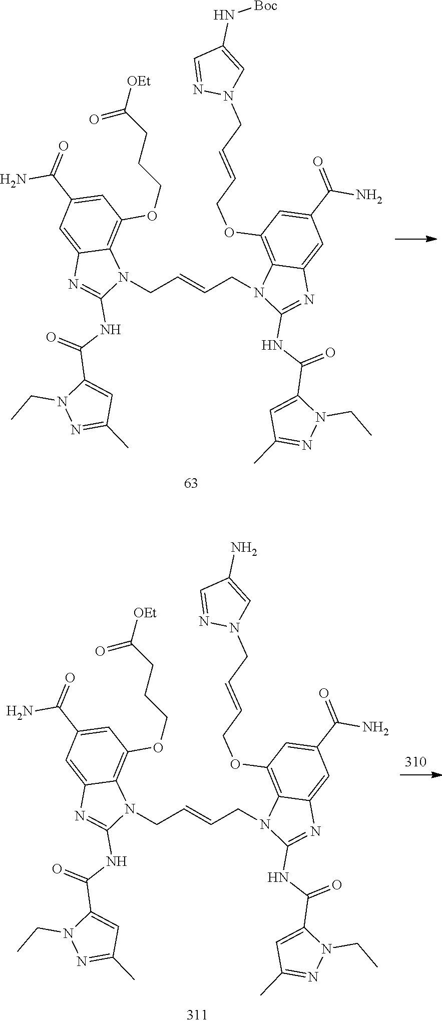

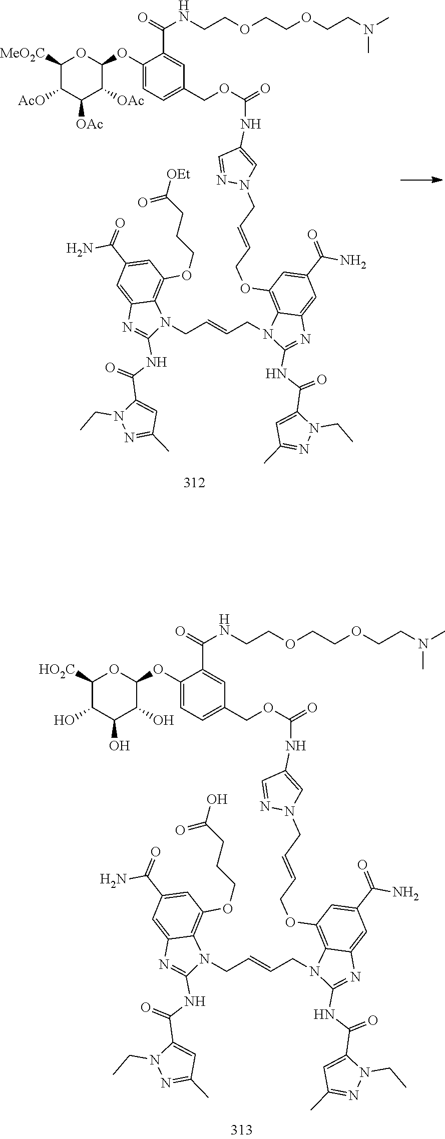 [SMILES: CCOC(=O)CCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC/C=C/Cn5cc(NC(=O)OCc6ccc(O[C@@H]7O[C@H](C(=O)OC)[C@@H](OC(C)=O)[C@H](OC(C)=O)[C@H]7OC(C)=O)c(C(=O)NCCOCCOCCN(C)C)c6)cn5)c43)c12 ; CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN(C)C)c4)cn3)c2n1C/C=C/Cn1c(NC(=O)c2cc(C)nn2CC)nc2cc(C(N)=O)cc(OCCCC(=O)O)c21]

### Preparation of Intermediate Compound 311

To a solution of Intermediate Compound 63 (500 mg, 0.09 mmol) in dichloromethane (3 mL) was added trifluoroacetic acid (1 mL) under nitrogen at 0° C. After stirred at room temperature for 1.5 hour, the reaction solution was concentrated under reduced pressure. The resulting residue was purified by reversed phase column chromatography to afford Intermediate Compound 311 (286 mg, 470%). EI-MS m/z: [M+H]⁺ 958.99.

### Preparation of Intermediate Compound 312

To a solution of Intermediate Compound 311 (286 mg, 0.22 mmol) in N,N-dimethylformamide (3 mL) were added Intermediate Compound 310 (213 mg, 0.26 mmol), N,N′-diisopropylethylamine (0.2 mL, 1.10 mmol) and 1-hydroxy-7-azabenzotriazole (HOAt, 3 mg, 0.02 mmol) at 0° C. under nitrogen. After stirred at room temperature for 21 hours, the reaction solution was concentrated under reduced pressure. The resulting residue was purified by reversed phase column chromatography to afford Intermediate Compound 312 (294 mg, 68%). EI-MS m/z: [M+H]⁺ 1626.61.

### Preparation of Compound 313

To a solution of Intermediate Compound 312 (294 mg) in tetrahydrofuran (1.5 mL) and methanol (1 mL) was added lithium hydroxide monohydrate (93 mg, 2.24 mmol) in distilled water (4 mL) at −70° C. under nitrogen. After stirred at 0° C. for 6 hours, the reaction mixture was adjusted to pH 4-5 with acetic acid and concentrated under reduced pressure. The resulting residue was purified by HPLC to afford Compound 313 (115 mg, 42%).

¹H-NMR (400 MHz, MeOD) δ 7.87 (d, J=2.3 Hz, 1H), 7.66 (s, 1H), 7.58 (dd, J=6.9, 1.3 Hz, 2H), 7.49-7.40 (m, 1H), 7.36 (s, 1H), 7.28-7.20 (m, 3H), 6.57 (d, J=15.2 Hz, 2H), 5.94-5.60 (m, 4H), 5.09 (d, J=4.9 Hz, 2H), 4.96 (t, J=15.4 Hz, 2H), 4.87 (s, 2H), 4.77 (s, 4H), 4.56 (q, J=6.4 Hz, 4H), 4.48 (d, J=5.4 Hz, 2H), 4.05 (d, J=9.5 Hz, 1H), 3.92 (t, J=6.4 Hz, 2H), 3.79 (dd, J=5.7, 4.4 Hz, 2H), 3.70 (s, 2H), 3.69-3.48 (m, 4H), 2.84 (s, 6H), 2.31 (t, J=7.3 Hz, 2H), 2.18 (d, J=6.3 Hz, 6H), 1.84 (p, J=6.9 Hz, 2H), 1.33-1.29 (m, 6H). EI-MS m/z: [M+H]⁺ 1459.85.

### Example 52: Preparation of Compound 316

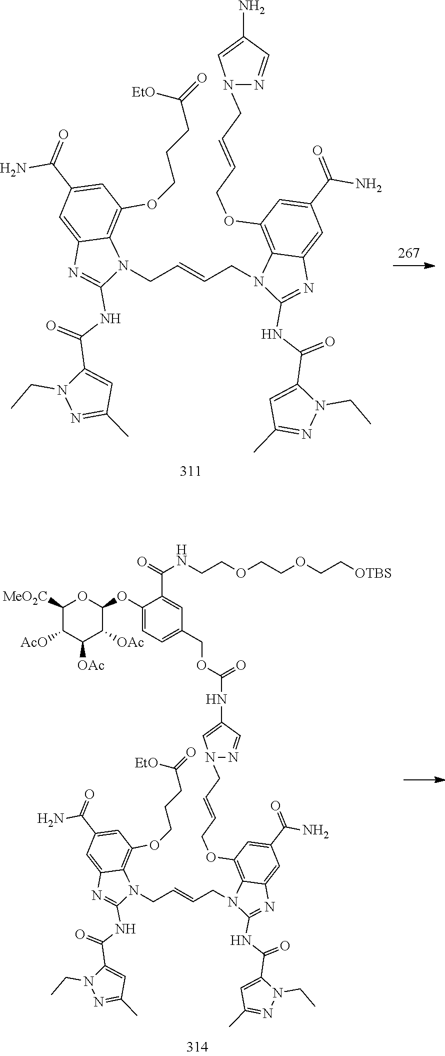 [SMILES: CCOC(=O)CCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC/C=C/Cn5cc(N)cn5)c43)c12 ; CCOC(=O)CCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC/C=C/Cn5cc(NC(=O)OCc6ccc(O[C@@H]7O[C@H](C(=O)OC)[C@@H](OC(C)=O)[C@H](OC(C)=O)[C@H]7OC(C)=O)c(C(=O)NCCOCCOCCO[Si](C)(C)C(C)(C)C)c6)cn5)c43)c12]

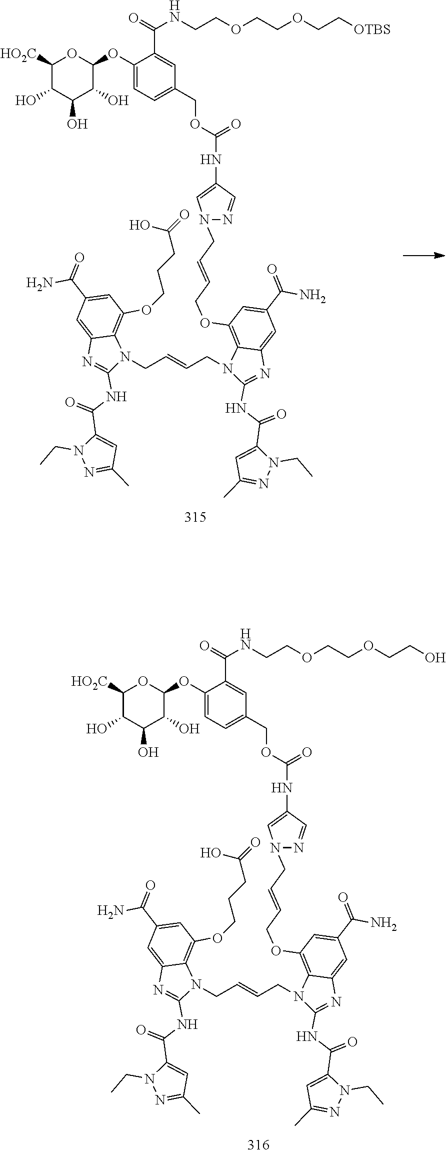 [SMILES: CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCO[Si](C)(C)C(C)(C)C)c4)cn3)c2n1C/C=C/Cn1c(NC(=O)c2cc(C)nn2CC)nc2cc(C(N)=O)cc(OCCCC(=O)O)c21 ; CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCO)c4)cn3)c2n1C/C=C/Cn1c(NC(=O)c2cc(C)nn2CC)nc2cc(C(N)=O)cc(OCCCC(=O)O)c21]

### Preparation of Intermediate Compound 314

To a solution of Intermediate Compound 311 (120 mg, 0.09 mmol) in N,N-dimethylformamide (1 mL) were added Intermediate Compound 267 (104 mg, 0.11 mmol), N,N′-diisopropylethylamine (0.1 mL, 0.59 mmol) at 0° C. under nitrogen. After stirred at room temperature for 2 hours, the reaction solution was concentrated under reduced pressure to afford Intermediate Compound 314 (crude), which was used without further purification. EI-MS m/z: [M/2+H]⁺ 857.39.

### Preparation of Intermediate Compound 315

To a solution of Intermediate Compound 314 (139 mg, 0.08 mmol) in tetrahydrofuran (1.3 mL) and methanol (1.3 mL) was added lithium hydroxide monohydrate (17 mg, 0.41 mmol) in distilled water (1.3 mL) at −70° C. under nitrogen. After stirred at 0° C. for 6 hours, the reaction mixture was adjusted to pH 4-5 with acetic acid and concentrated under reduced pressure to afford Intermediate Compound 315 (crude), which was used without further purification. EI-MS m/z: [M/2+H]⁺ 773.34.

### Preparation of Compound 316

To a solution of Intermediate Compound 315 (121 mg, 0.08 mmol) in dichloromethane (3 mL) was added trifluoroacetic acid (0.6 mL) at 0° C. under nitrogen. After stirred at room temperature for 2 hours, the reaction solution was concentrated under reduced pressure. The resulting residue was purified by HPLC to afford Compound 316 (58.9 mg, 45%). EI-MS m/z: [M+H]⁺ 1431.89, [M/2+H]⁺716.28.

### Example 53: Preparation of Compound 320

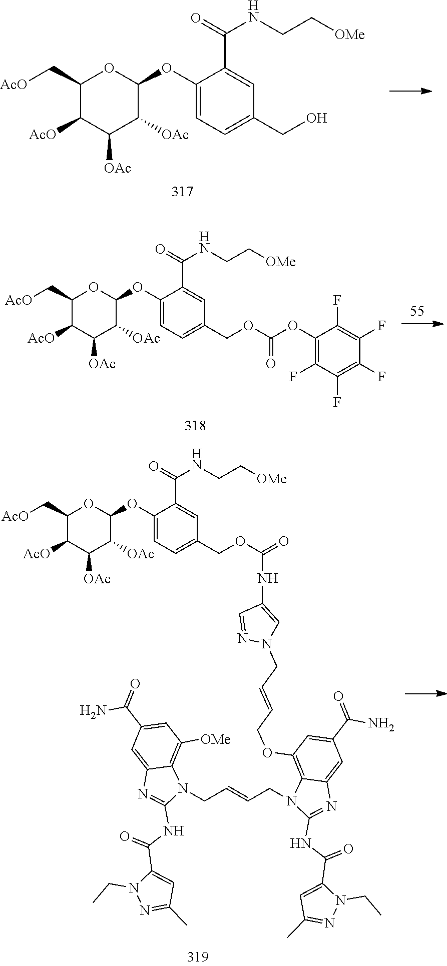 [SMILES: COCCNC(=O)c1cc(CO)ccc1O[C@@H]1O[C@H](COC(C)=O)[C@H](OC(C)=O)[C@H](OC(C)=O)[C@H]1OC(C)=O ; COCCNC(=O)c1cc(COC(=O)Oc2c(F)c(F)c(F)c(F)c2F)ccc1O[C@@H]1O[C@H](COC(C)=O)[C@H](OC(C)=O)[C@H](OC(C)=O)[C@H]1OC(C)=O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](COC(C)=O)[C@H](OC(C)=O)[C@H](OC(C)=O)[C@H]5OC(C)=O)c(C(=O)NCCOC)c4)cn3)c21]

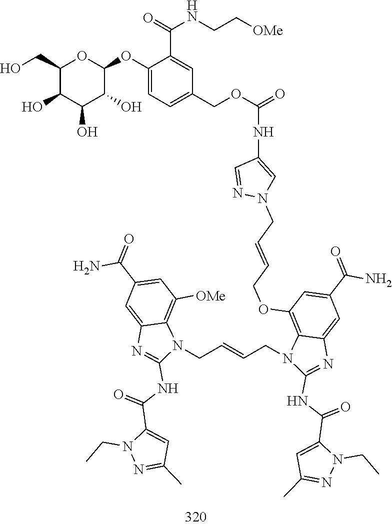 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](CO)[C@H](O)[C@H](O)[C@H]5O)c(C(=O)NCCOC)c4)cn3)c21]

### Preparation of Intermediate Compound 318

To a solution of Intermediate Compound 317 (1.6 g, 2.90 mmol, Intermediate Compound 317 was prepared according to the method described in the International patent publication No. WO 2018/182341 A1) in N,N-dimethylformamide (10 mL) were added bis(pentafluorophenyl)carbonate (1.4 g, 3.48 mmol) and N,N-diisopropylethylamine (1.03 ml, 5.80 mmol) at 0° C. under nitrogen. After stirred at room temperature for 17 hours, the reaction solution was diluted with ethyl acetate (100 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 318 (2.0 g, 90%). EI-MS m/z: [M+H]⁺ 765.89.

### Preparation of Intermediate Compound 319

To a solution of Intermediate Compound 318 (25 mg, 0.021 mmol) in N,N-dimethylformamide (1 mL) were added Intermediate Compound 55 (17 mg, 0.022 mmol), N,N′-diisopropylethylamine (0.02 mL, 0.104 mmol) and 1-hydroxy-7-azabenzotriazole (HOAt, 0.8 mg, 0.006 mmol) at 0° C. under nitrogen. After stirred at room temperature for 3 hours, the reaction solution was concentrated under reduced pressure to afford Intermediate Compound 319 (32 mg, Crude), which was used without further purification. EI-MS m/z: [M12+H]⁺ 1440.26.

### Preparation of Compound 320

To a solution of Intermediate Compound 320 (32 mg, Crude) in tetrahydrofuran (0.5 mL) and methanol (0.5 mL) was added lithium hydroxide monohydrate (9.3 mg, 9.33 mmol) in distilled water (0.5 mL) at −70° C. under nitrogen. After stirred at 0° C. for 3 hours, the reaction mixture was adjusted to pH 4-5 with acetic acid and concentrated under reduced pressure. The resulting residue was purified by HPLC to afford Compound 320 (23 mg). EI-MS m/z: [M+H]⁺ 1271.95.

### Example 54: Preparation of Compound 328

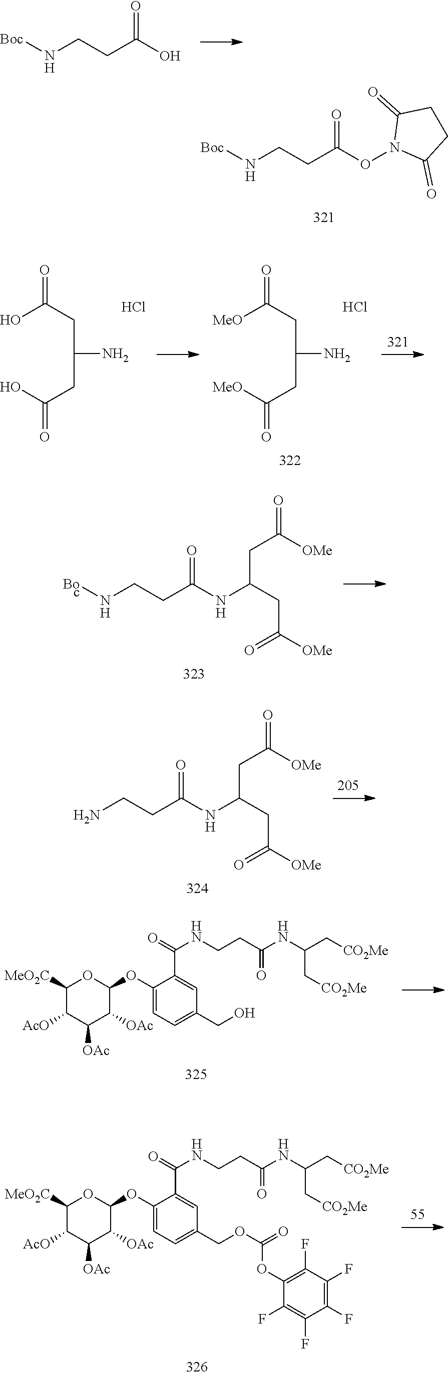

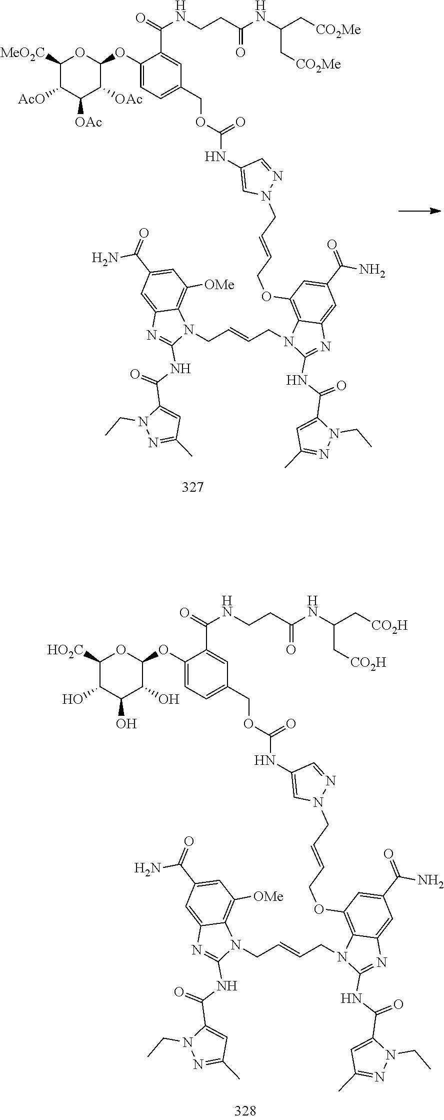 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)OC)[C@@H](OC(C)=O)[C@H](OC(C)=O)[C@H]5OC(C)=O)c(C(=O)NCCC(=O)NC(CC(=O)OC)CC(=O)OC)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCC(=O)NC(CC(=O)O)CC(=O)O)c4)cn3)c21]

### Preparation of Intermediate Compound 321

To a solution of 3-((t-butylcarbonyl)amino)propanoic acid (500 mg, 2.64 mmol) in dichloromethane (20 mL) were added N-(3-dimethylaminopropyl)-N′-ethylcarbodiimide hydrochloride (EDC HCl, 760 mg, 3.96 mmol) and N-hydroxysuccinimide (423 mg, 3.96 mmol). After stirred at room temperature for 17 hours, the reaction solution was diluted with dichloromethane (50 mL) and then washed with saturated aqueous sodium bicarbonate solution (50 mL) and distilled water (50 mL). The organic layer was dried over anhydrous sodium sulfate and filtered. The filtrate was concentrated under reduced pressure to afford Intermediate Compound 321 (806 mg, Crude), which was used without further purification.

¹H-NMR (400 MHz, CDCl₃) δ 5.10 (s, 1H), 3.55-3.50 (m, 2H), 2.85-2.80 (m, 6H), 1.45 (s, 9H).

### Preparation of Intermediate Compound 322

To a solution of #-glutamic acid hydrochloride (1 g, 5.45 mmol) in methanol (30 mL) was added thionyl chloride (2 mL, 27.23 mmol) at 0° C. under nitrogen. After stirred at room temperature for 4 hours, the reaction solution was concentrated under reduced pressure to afford Intermediate Compound 322 (1.17 g, crude), which was used without further purification.

¹H-NMR (400 MHz, MeOD) δ 3.75 (s, 6H), 2.89-2.71 (m, 4H).

### Preparation of Intermediate Compound 323

To a solution Intermediate Compound 322 (493 mg, 2.82 mmol) in dichloromethane (20 mL) were added N,N-diisopropylethylamine (1.47 mL, 8.45 mmol) and Intermediate Compound 321 (806 mg, 2.82 mmol) at 0° C. under nitrogen. After stirred at room temperature for 17 hours, the reaction solution was diluted with dichloromethane (100 mL) and then washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 323 (903 mg, 92%).

¹H-NMR (400 MHz, CDCl₃) δ 5.19 (s, 1H), 4.68-4.58 (m, 1H), 3.70 (s, 6H), 3.39 (d, J=6.6 Hz, 2H), 2.76-2.58 (m, 4H), 2.36 (t, J=6.0 Hz, 2H), 1.44 (s, 9H).

### Preparation of Intermediate Compound 324

To a solution of Intermediate Compound 323 (903 mg, 2.61 mmol) in dichloromethane (10 mL) was added hydrogen chloride (4M 1,4-dioxane solution. 5 mL). After stirred at room temperature for 2 hours, the reaction solution was concentrated under reduced pressure to afford Intermediate Compound 324 (845 mg, Crude), which was used without further purification. EI-MS m/z: [M+H]⁺ 247.22.

### Preparation of Intermediate Compound 325

To a solution of Intermediate Compound 205 (1.0 g, 2.06 mmol, Intermediate Compound 205 was prepared by the method described in the International patent publication No. WO 2018/182341 A1) in dichloromethane (20 mL) were added N-(3-dimethylaminopropyl)-N′-ethylcarbodiimide hydrochloride (EDC HCl, 415 mg, 2.27 mmol), 1-hydroxybenzotriazole (HOBt, 306 mg, 2.27 mmol), Intermediate Compound 324 (700 mg, 2.48 mmol), and N-methylmorpholine (0.68 mL, 6.19 mmol) at 0° C. under nitrogen. After stirred at room temperature for 2 hours, the reaction solution was diluted with dichloromethane (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 325 (1.03 mg, 70%).

¹H-NMR (400 MHz, DMSO-d₆) δ 7.98-7.88 (m, 2H), 7.59 (s, 1H), 7.39 (d, J=8.5 Hz, 1H), 7.11 (d, J=8.5 Hz, 1H), 5.71 (d, J=7.9 Hz, 1H), 5.49 (t, J=9.6 Hz, 1H), 5.28-5.18 (m, 2H), 5.09 (t, J=9.8 Hz, 1H), 4.74 (d, J=9.9 Hz, 1H), 4.46 (d, J=5.2 Hz, 2H), 4.39 (d, J=7.8 Hz, 1H), 3.65 (s, 3H), 3.57 (s, 6H), 2.33-2.28 (m, 4H), 2.01 (s, 9H).

### Preparation of Intermediate Compound 326

To a solution of Intermediate Compound 325 (1.03 g, 1.45 mmol) in dichloromethane (30 mL) were added bis(pentafluorophenyl)carbonate (627 mg, 1.59 mmol) and N,N-diisopropylethylamine (0.38 mL, 2.17 mmol) at 0° C. under nitrogen. After stirred at room temperature for 5 hours, the reaction solution diluted with dichloromethane (100 mL) and washed with saturated aqueous sodium bicarbonate solution (50 mL). The organic layer was dried over anhydrous sodium sulfate and filtered. The filtrate was concentrated under reduced pressure. The resulting residue was purified by column chromatography to afford Intermediate Compound 326 (92 mg, 39%). EI-MS m/z: [M+H]⁺ 922.89.

### Preparation of Intermediate Compound 327

To a solution of Intermediate Compound 55 (50 mg, 0.04 mmol) in N,N-dimethylformamide (1 mL) were added Intermediate Compound 326 (40 mg, 0.04 mmol), N,N′-diisopropylethylamine (0.04 mL, 0.21 mmol) at 0° C. under nitrogen. After stirred at room temperature for 3 hours, the reaction solution was concentrated under reduced pressure to afford Intermediate Compound 327 (70 mg, Crude), which was used without further purification. EI-MS m/z: [M+H]⁺ 1596.94.

### Preparation of Compound 328

To a solution of Intermediate Compound 327 (70 mg, Crude) in tetrahydrofuran (1 mL) and methanol (1 mL) was added lithium hydroxide monohydrate (34.7 mg, 0.83 mmol) in distilled water (1 mL) at −45° C. under nitrogen. After stirred at 0° C. for 1 hours, the reaction mixture was adjusted to pH 4-5 with acetic acid. and concentrated under reduced pressure. The resulting residue was purified by HPLC to afford Compound 328 (39 mg). EI-MS m/z: [M+H]⁺ 1428.89.

### Example 55: Preparation of Compound 330

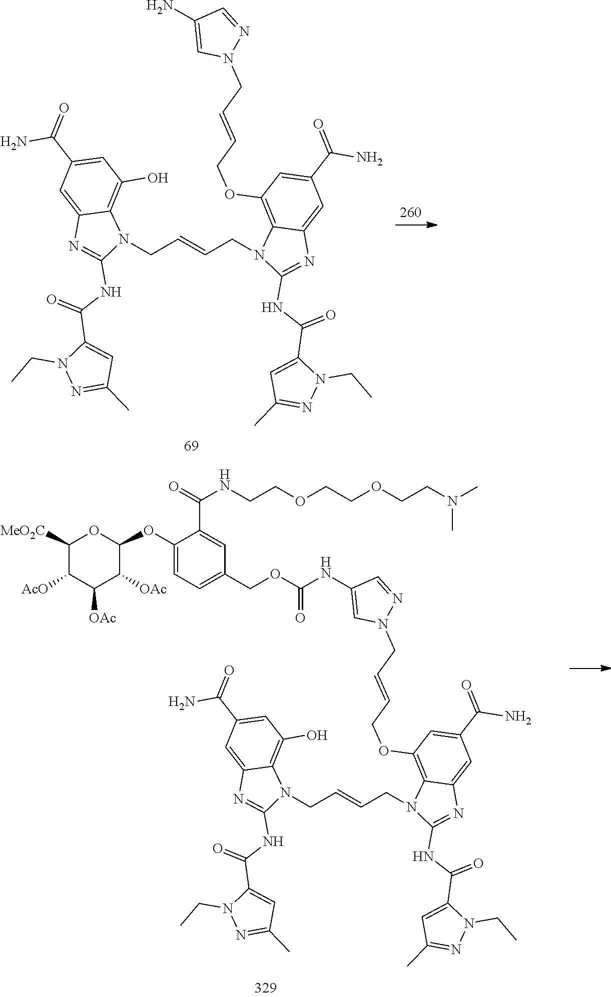 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC/C=C/Cn3cc(N)cn3)c21 ; CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)OC)[C@@H](OC(C)=O)[C@H](OC(C)=O)[C@H]5OC(C)=O)c(C(=O)NCCOCCOCCN(C)C)c4)cn3)c21]

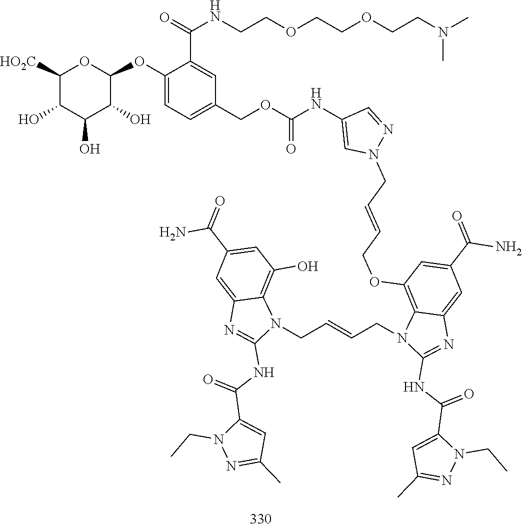 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN(C)C)c4)cn3)c21]

### Preparation of Intermediate Compound 329

To a solution of Intermediate Compound 69 (100 mg, 0.08 mmol) in N,N-dimethylformamide (2 mL) were added Intermediate Compound 260 (82 mg, 0.40 mmol), N,N′-diisopropylethylamine (0.08 mL, 0.42 mmol) and 1-hydroxy-7-azabenzotriazole (HOAt, 2.0 mg, 0.02 mmol) at 0° C. under nitrogen. After stirred at room temperature for 20 hours, the reaction solution was concentrated under reduced pressure and purified by reversed phase column chromatography to afford Intermediate Compound 329 (58 mg). EI-MS m/z: [M+H]⁺ 1513.05.

### Preparation of Compound 330

To a solution of Intermediate Compound 329 (58 mg, crude) in tetrahydrofuran (0.5 mL) and methanol (0.5 mL) was added lithium hydroxide monohydrate (6.4 mg, 0.15 mmol) in distilled water (0.5 mL) at −70° C. under nitrogen. After stirred at 0° C. for 3 hours, the reaction mixture was adjusted to pH 4-5 with acetic acid and concentrated under reduced pressure. The resulting residue was purified by HPLC to afford Compound 330 (17 mg, 32%). EI-MS m/z: [M+H]⁺ 1428.89.

### Example 56: Preparation of Compound 332

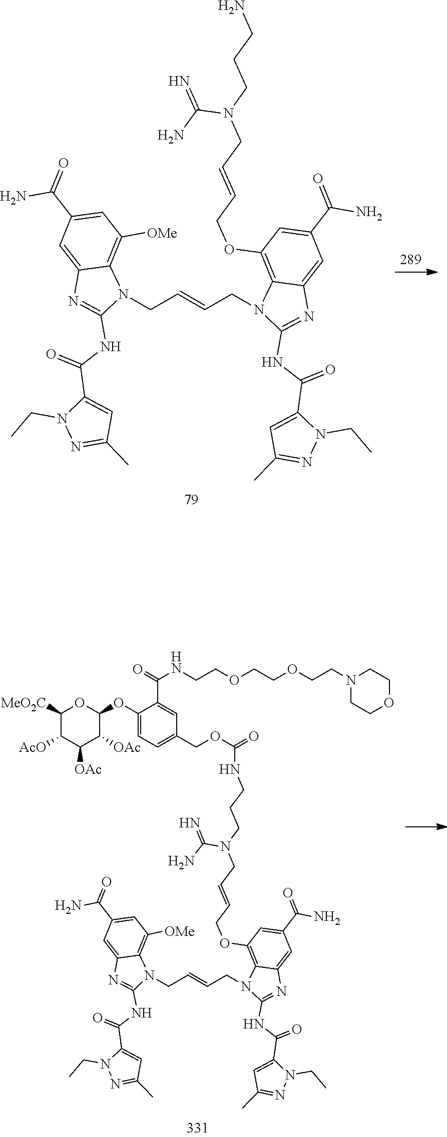 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN(CCCN)C(=N)N)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN(CCCNC(=O)OCc3ccc(O[C@@H]4O[C@H](C(=O)OC)[C@@H](OC(C)=O)[C@H](OC(C)=O)[C@H]4OC(C)=O)c(C(=O)NCCOCCOCCN4CCOCC4)c3)C(=N)N)c21]

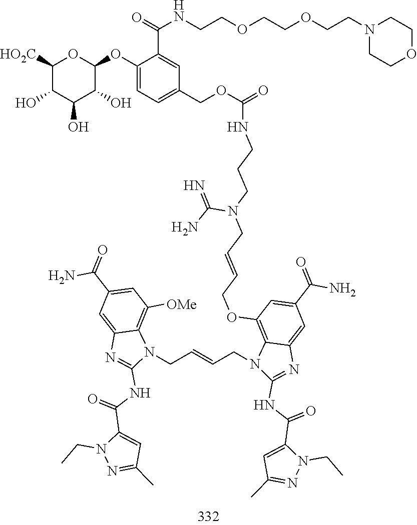 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN(CCCNC(=O)OCc3ccc(O[C@@H]4O[C@H](C(=O)O)[C@@H](O)[C@H](O)[C@H]4O)c(C(=O)NCCOCCOCCN4CCOCC4)c3)C(=N)N)c21]

### Preparation of Intermediate Compound 331

To a solution of Intermediate Compound 79 (35 mg, 0.028 mmol) in N,N-dimethylformamide (1 mL) were added Intermediate Compound 289 (25 mg, 0.028 mmol), N,N′-diisopropylethylamine (0.02 mL, 0.142 mmol) at 0° C. under nitrogen. After stirred at room temperature for 6 hours, the reaction solution was concentrated under reduced pressure to afford Intermediate Compound 331 (45 mg, Crude), which was used without further purification. EI-MS m/z: [M+H]⁺ 1601.84.

### Preparation of Compound 332

To a solution of Intermediate Compound 331 (45 mg, Crude) in tetrahydrofuran (0.5 mL) and methanol (0.5 mL) was added lithium hydroxide monohydrate (12 mg, 0.28 mmol) in distilled water (0.5 mL) at −70° C. under nitrogen. After stirred at 0° C. for 3 hours, the reaction mixture was adjusted to pH 4-5 with acetic acid and concentrated under reduced pressure. The resulting residue was purified by HPLC to afford Compound 332 (32 mg). EI-MS m/z: [M+H]⁺ 1461.96.

### Example 57: Preparation of Compound 337

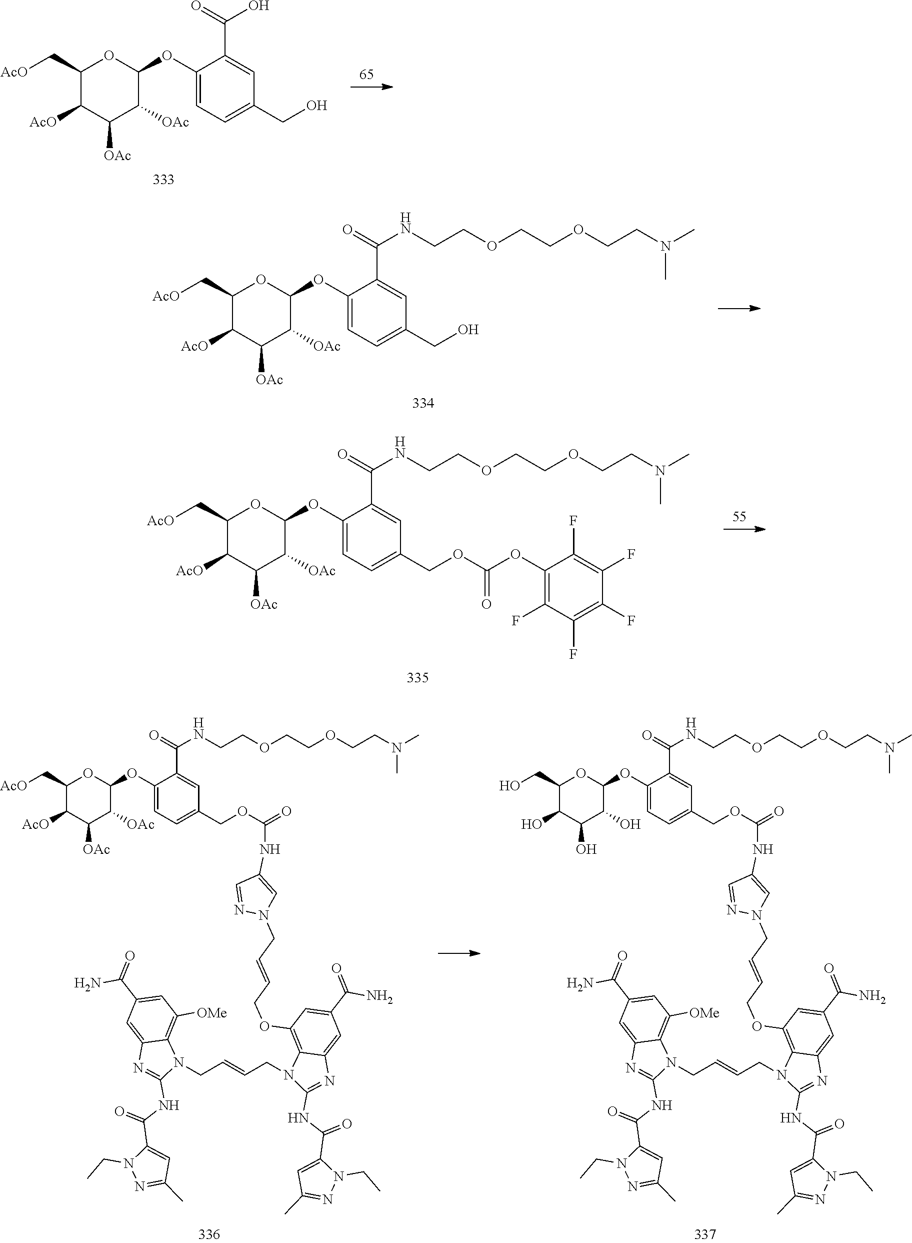

### Preparation of Intermediate Compound 334

To a solution of Intermediate Compound 333 (300 mg, 0.60 mmol, Intermediate Compound 333 was prepared by the method described in the International patent publication No. WO 2018/182341 A1) in N,N-dimethylformamide (3 mL) were added N,N,N′,N′-tetramethyl-O-(1H-benzotriazol-1-yl)uronium hexafluorophosphate (HBTU, 297 mg, 0.78 mmol), Intermediate Compound 65 (127 mg, 0.72 mmol) and N,N′-diisopropylethylamine (0.20 mL, 1.20 mmol) at 0° C. under nitrogen. After stirred at room temperature for 17 hours, the reaction solution was concentrated under reduced pressure. The resulting residue was purified by column chromatography to afford Intermediate Compound 334 (190 mg, 48%). EI-MS m/z: [M+H]⁺ 657.14.

### Preparation of Intermediate Compound 335

To a solution of Intermediate Compound 334 (57 mg, 0.09 mmol) in dichloromethane (3 mL) were added bis(pentafluorophenyl)carbonate (34 mg, 0.09 mmol) and N,N-diisopropylethylamine (0.01 mL, 0.26 mmol) at 0° C. under nitrogen. After stirred at room temperature for 3 hours, the reaction solution was concentrated under reduced pressure, to afford Intermediate Compound 335 (crude), which was used without further purification. EI-MS m/z: [M+H]⁺ 867.76.

### Preparation of Intermediate Compound 336

To a solution of Intermediate Compound 335 (50 mg, 0.06 mmol) in dichloromethane (3 mL) were added Intermediate Compound 55 (60 mg, 0.07 mmol), N,N′-diisopropylethylamine (0.03 mL, 0.17 mmol) and 1-hydroxy-7-azabenzotriazole (HOAt, 1.6 mg, 0.01 mmol) at 0° C. under nitrogen. After stirred at room temperature for 18 hours, the reaction solution was concentrated under reduced pressure. The resulting residue was purified by reversed phase column chromatography to afford Intermediate Compound 336 (36 mg, 40%). EI-MS m/z: [M+H]⁺ 1450.92.

### Preparation of Compound 337

To a solution of Intermediate Compound 336 (36 mg, Crude) in tetrahydrofuran (1 mL) and methanol (1 mL) was added lithium hydroxide monohydrate (10 mg, 0.24 mmol) in distilled water (1 mL) at −70° C. under nitrogen. After stirred at 0° C. for 2 hours, the reaction mixture was adjusted to pH 4-5 with acetic acid and concentrated under reduced pressure. The resulting residue was purified by HPLC to afford Compound 337 (10 mg, 30%). EI-MS m/z: [M+H]⁺ 1372.04.

### Example 58: Preparation of Compound 344

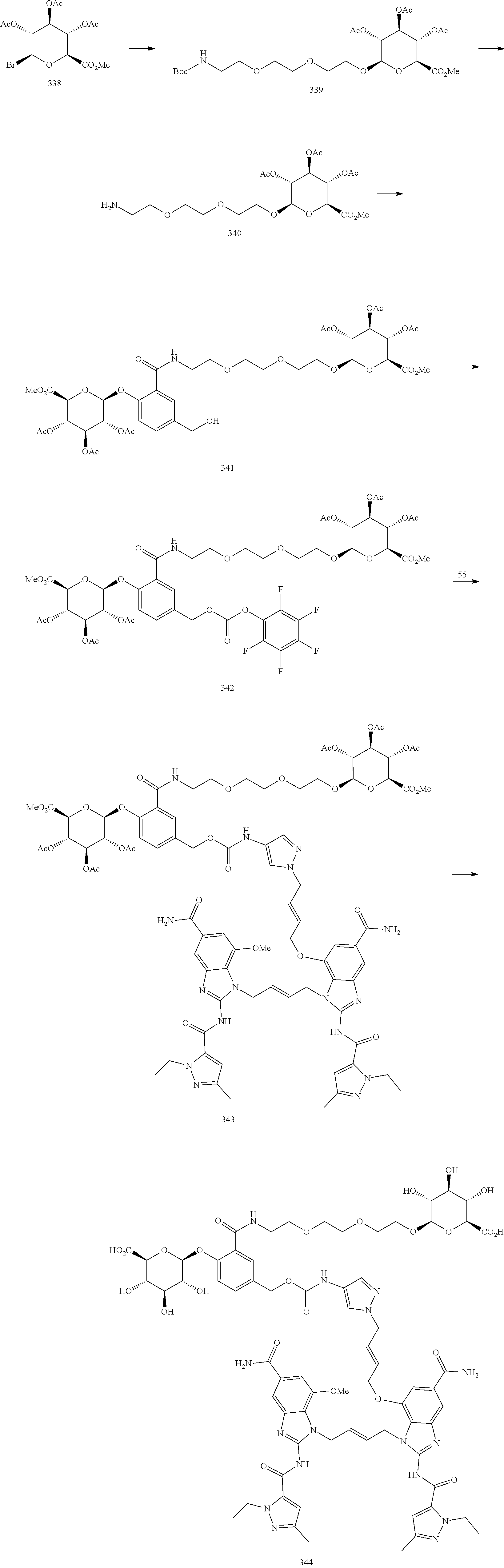

### Preparation of Intermediate Compound 339

To a solution of Intermediate Compound 338 (300 mg, 0.755 mmol, Intermediate Compound 338 was prepared by the method described in the International patent publication No. WO 2018/182341 A1) in dichloromethane (5 mL) were added Molecular Sieve (500 mg), t-butyl (2-(2-(2-hydroxyethoxy)ethoxy)ethyl)carbamate (226 mg, 0.91 mmol) and trifluoromethanesulfonate (233 mg, 0.91 mmol) at 0° C. under nitrogen. After stirred at room temperature for 40 minutes, the reaction solution was diluted with dichloromethane (100 mL) and washed with distilled water (50 mL). The organic layer was dried over anhydrous sodium sulfate and filtered. The filtrate was concentrated under reduced pressure. The resulting residue was purified by column chromatography to afford Intermediate Compound 339 (150 mg, 35%). EI-MS m/z: [M+H]⁺ 566.19.

### Preparation of Intermediate Compound 340

To a solution of Intermediate Compound 339 (150 mg, 0.265 mmol) in dichloromethane (1 mL) was added trifluoroacetic acid (0.1 mL) at 0° C. under nitrogen. After stirred at room temperature for 4 hours, the reaction solution was concentrated under reduced pressure to afford Intermediate Compound 340 (153 mg, crude), which was used without further purification. EI-MS m/z: [M+H]⁺ 466.19.

### Preparation of Intermediate Compound 341

To a solution of Intermediate Compound 338 (154 mg, 0.32 mmol, Intermediate Compound 338 was prepared by the method described in the International patent publication No. WO 2018/182341 A1) in N,N-dimethylformamide (1 mL) were added N-(3-dimethylaminopropyl)-N′-ethylcarbodiimide hydrochloride (EDC HCl, 53.38 mg, 0.28 mmol), 1-hydroxybenzotriazole (HOBt, 39.4 mg, 0.29 mmol), Intermediate Compound 185 (153 mg), and N-methylmorpholine (0.07 mL, 0.66 mmol) at 0° C. under nitrogen. After stirred at room temperature for 15 hours, the reaction solution was diluted with ethyl acetate (100 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 341 (139 mg, 56%). EI-MS m/z: [M+H]⁺ 932.01.

### Preparation of Intermediate Compound 342

To a solution of Intermediate Compound 341 (130 mg, 0.14 mmol) in dichloromethane (1 mL) were added bis(pentafluorophenyl)carbonate (66 mg, 1.17 mmol) and N,N-diisopropylethylamine (0.07 ml, 0.42 mmol) at 0° C. under nitrogen. After stirred at room temperature for 20 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 342 (159 mg, 99%). EI-MS m/z: [M+H]⁺ 1141.92.

### Preparation of Intermediate Compound 343

To a solution of Intermediate Compound 55 (184 mg, 0.15 mmol) in N,N-dimethylformamide (1 mL) were added Intermediate Compound 187 (159 mg, 0.14 mmol) and N,N′-diisopropylethylamine (0.12 mL, 0.7 mmol) at 0° C. under nitrogen. After stirred at room temperature for 3 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 343 (90 mg, 36%). EI-MS m/z: [M+H]⁺ 908.59.

### Preparation of Compound 344

To a solution of Intermediate Compound 341 (90 mg, 0.05 mmol) in tetrahydrofuran (1 mL) and methanol (1 mL) was added lithium hydroxide monohydrate (20.8 mg, 0.5 mmol) in distilled water (1 mL) at −70° C. under nitrogen. After stirred at 0° C. for 2 hours, the reaction mixture was adjusted to pH 4-5 with acetic acid and concentrated under reduced pressure. The resulting residue was purified by HPLC to afford Compound 344 (13.8 mg, 18%). EI-MS m/z: [M+H]⁺ 1535.25.

### Example 59: Preparation of Compound 350

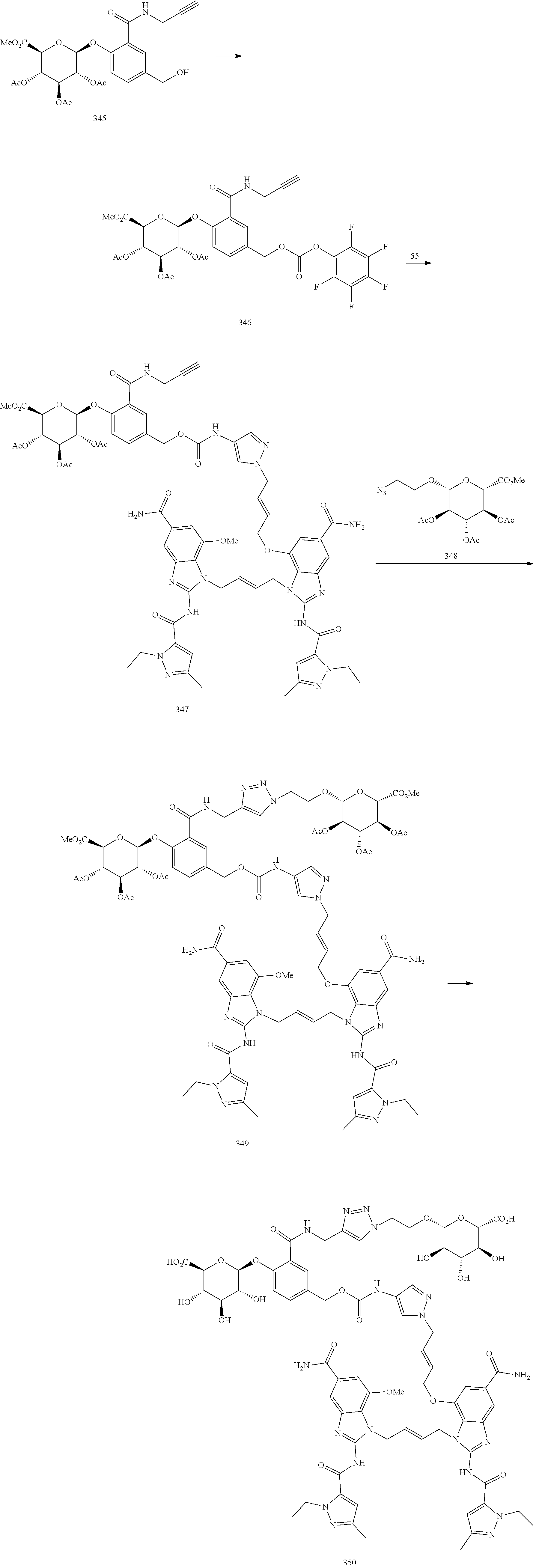

### Preparation of Intermediate Compound 346

To a solution of Intermediate Compound 345 (2 g, 3.84 mmol) in dichloromethane (20 mL) were added bis(pentafluorophenyl)carbonate (1.65 g, 4.99 mmol) and N,N-diisopropylethylamine (2 ml, 11.51 mmol) at 0° C. under nitrogen. After stirred at room temperature for 3 hours, the reaction solution was diluted with dichloromethane (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 346 (2 g, 71%).

¹H-NMR (400 MHz, DMSO) δ 7.96 (d, J=6.4 Hz, 1H), 7.74 (s, 1H), 7.59 (d, J=8.6 Hz, 1H), 7.23 (d, J=8.6 Hz, 1H), 5.79 (d, J=7.9 Hz, 1H), 5.49 (t, J=9.8 Hz, 1H), 5.39 (s, 2H), 5.22 (t, J=8.9 Hz, 1H), 5.08 (t, J=9.7 Hz, 1H), 4.75 (d, J=10.4 Hz, 1H), 4.12 (d, J=2.5 Hz, 2H), 4.08-3.96 (m, 1H), 3.64 (d, J=2.2 Hz, 4H), 3.54 (q, J=5.1 Hz, 10H), 2.06-1.96 (m, 10H). EI-MS m/z: [M+H]⁺ 731.84.

### Preparation of Intermediate Compound 347

To a solution of Intermediate Compound 55 (108 mg, 0.09 mmol) in N,N-dimethylformamide (1 mL) were added Intermediate Compound 346 (26.2 mg, 0.03 mmol) and N,N′-diisopropylethylamine (0.02 mL, 0.13 mmol) at 0° C. under nitrogen. After stirred at room temperature for 6 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure to afford Intermediate Compound 347 (128 mg, crude), which was used without further purification. EI-MS m/z: [M+H]⁺ 1405.24.

### Preparation of Intermediate Compound 349

To a solution of Intermediate Compound 347 (124 mg, 0.091 mmol) in ethanol (2 mL), dichloromethane and distilled water (2 mL) were added Intermediate Compound 348 (37 mg, 0.091 mmol, Intermediate Compound 348 was prepared by the method described in the United States patent publication No. US 2010-0323973 A1), Tris(3-hydroxypropyltriazolymethyl)amine (THPTA, 4.7 mg, 0.04 mmol), Copper(II) sulfate pentahydrate (18.2 mg, 0.073 mmol) and ascorbic acid (24.1 mg, 0.136 mmol) at 0° C. under nitrogen. After stirred at room temperature for 6 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure, Intermediate to afford Compound 349 (164 mg, crude), which was used without further purification. EI-MS m/z: [M+H]⁺ 1808.89.

### Preparation of Compound 350

To a solution of Intermediate Compound 349 (164 mg, 0.09 mmol) in tetrahydrofuran (1 mL) and methanol (1 mL) was added lithium hydroxide monohydrate (17 mg, 0.41 mmol) in distilled water (1 mL) at −70° C. under nitrogen. After stirred at 0° C. for 1 hours, the reaction mixture was adjusted to pH 4-5 with acetic acid and concentrated under reduced pressure. The resulting residue was purified by HPLC to afford Compound 350 (16 mg, 11%). EI-MS m/z: [M/2+H]⁺1529.044.

### Example 60: Preparation of Compound 352

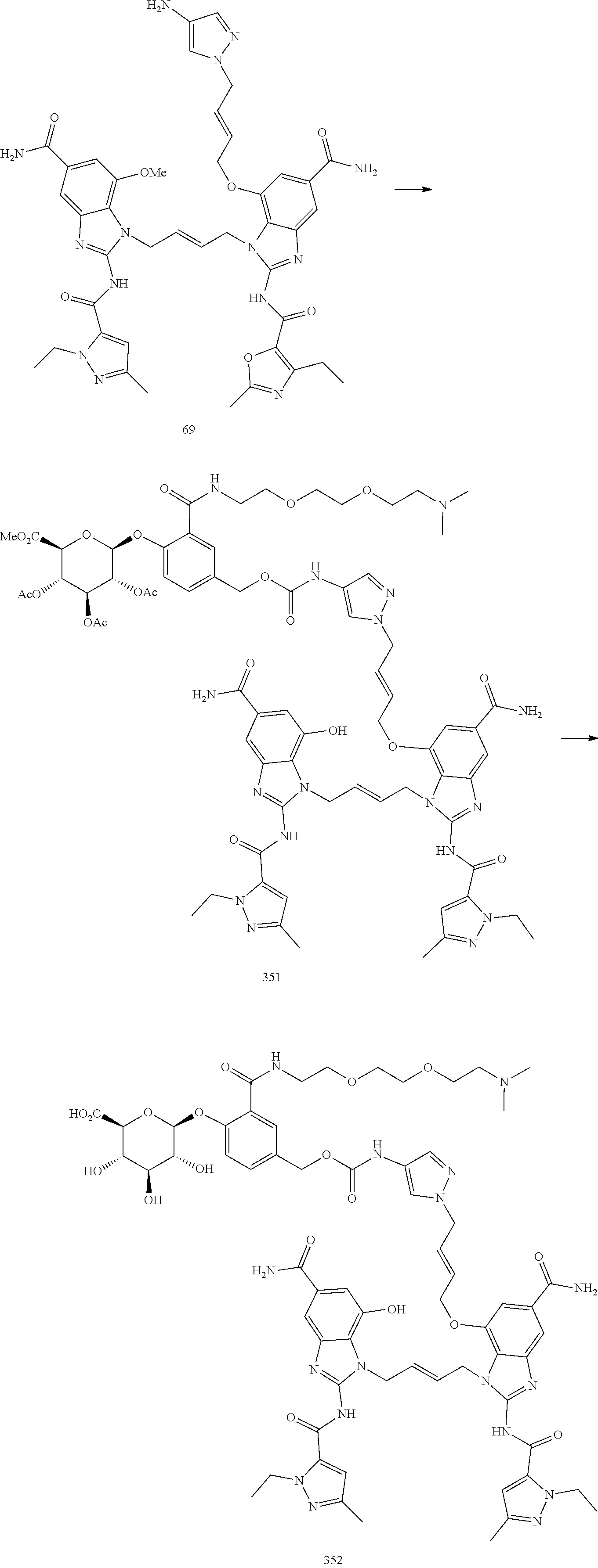

### Preparation of Intermediate Compound 351

To a solution of Intermediate Compound 69 (100 mg, 0.08 mmol) and Intermediate Compound 310 (82 mg, 0.10 mmol) in N,N-dimethylformamide (2 mL) were added 1-hydroxy-7-azabenzotriazole (HOAt, 2 mg, 0.02 mmol) and N,N′-diisopropylethylamine (0.08 mL, 0.42 mmol) at 0° C. under nitrogen. After stirred at room temperature for 20 hours under nitrogen, the reaction solution was concentrated under reduced pressure. The resulting residue was purified by reverse phase column chromatography to afford Intermediate Compound 351 (58 mg). EI-MS m/z: [M+H]⁺ 1513.05.

### Preparation of Compound 352

To a solution of Intermediate Compound 351 (58 mg) in tetrahydrofuran (1 mL) and methanol (1 mL) was added lithium hydroxide monohydrate (38 mg, 0.91 mmol) in distilled water (1 mL) at −70° C. After stirred for 6 hours at room temperature, the reaction mixture was adjusted to pH 4-5 with acetic acid. The reaction solution was concentrated under reduced pressure. The resulting residue was purified by HPLC to afford Compound 352 (17 mg, 2-steps 15%). EI-MS m/z: [M+H]⁺ 1372.54.

### Example 61: Preparation of Compound 357

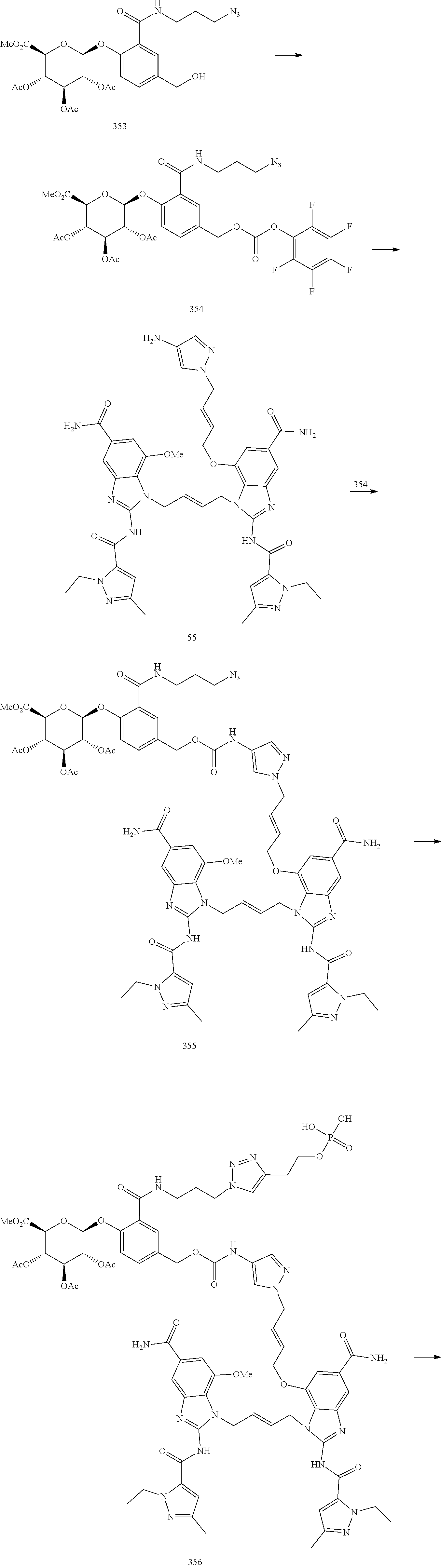

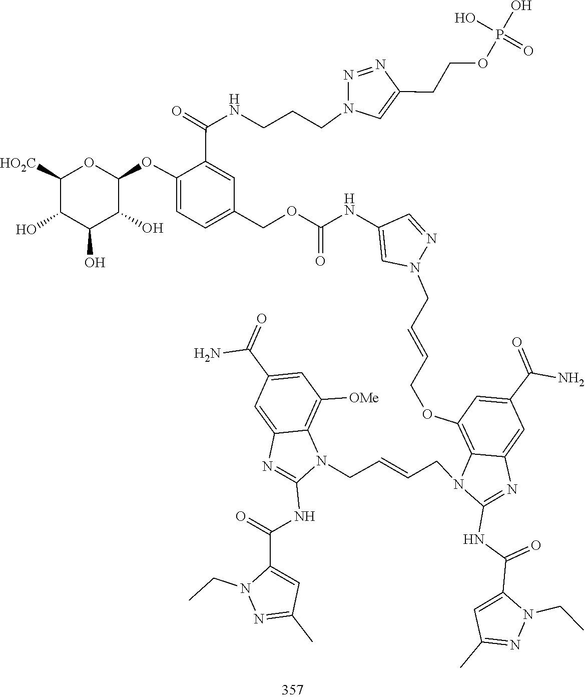 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCCn5cc(CCOP(=O)(O)O)nn5)c4)cn3)c21]

### Preparation of Intermediate Compound 354

To a solution of Intermediate Compound 353 (300 mg, 0.52 mmol, Intermediate Compound 353 was prepared by the method described in the International patent publication No. WO 2018/182341 A1) in dichloromethane (10 mL) were added bis(pentafluorophenyl)carbonate (417 mg, 1.06 mmol) and N, N′-diisopropyl ethyl amine (0.27 mL, 1.58 mmol) at 0° C. under nitrogen. After stirred at room temperature for 3 hours, the reaction solution was diluted with dichloromethane (30 mL) and washed with distilled water (30 mL×2). The organic layer was dried over anhydrous sodium sulfate and filtered. The filtrate was concentrated under reduced pressure. The resulting residue was purified by column chromatography to afford Intermediate Compound 354 (402 mg, 97%). EI-MS m/z: [M+H]⁺ 777.54.

### Preparation of Intermediate Compound 355

To a solution of Intermediate Compound 55 (100 mg, 0.11 mmol) and Intermediate Compound 354 (108 mg, 0.08 mmol) in N,N-dimethylformamide (1 mL) was added N,N′-diisopropylethylamine (0.06 mL, 0.35 mmol) at 0° C. under nitrogen. After stirred at room temperature for 6 hours, the reaction solution was diluted with ethyl acetate (30 mL) and washed with saturated aqueous sodium bicarbonate solution (30 mL) and distilled water (30 mL). The organic layer was dried over anhydrous sodium sulfate. After filtered and concentrated under reduced pressure to afford Intermediate Compound 355 (128 mg. crude), which was used without further purification. EI-MS m/z: [M+H]⁺ 1450.47.

### Preparation of Intermediate Compound 356

To a solution of Intermediate Compound 355 (64 mg) and but-3-yn-1-yl dihydrogen phosphate (299 mg, 1.10 mmol) in ethanol (2 mL), dichloromethane (2 mL) and distilled water (2 mL) were added tris(3-hydroxypropyltriazolylmethyl)amine (THPTA, 4.4 mg, 0.02 mmol), copper(II) sulfate pentahydrate (18.2 mg, 0.073 mmol) and sodium ascorbate (3.5 mg, 0.02) at 0° C. under nitrogen. After stirred at room temperature for 6 hours, the reaction solution was diluted with ethyl acetate (50 mL) and washed with distilled water (50 mL). The organic layer was dried over anhydrous sodium sulfate. After filtered and concentrated under reduced pressure to afford Intermediate Compound 356 (72 mg, crude), which was used without further purification. EI-MS m/z: [M+H]⁺ 1601.37.

### Preparation of Compound 357

To a solution of Intermediate Compound 356 (72 mg) in tetrahydrofuran (1 mL) and methanol (1 mL) was added lithium hydroxide monohydrate (70.8 mg, 0.49 mmol) in distilled water (1 mL) at −45° C. After stirred for 1 hours at room temperature, the reaction mixture was adjusted to pH 4-5 with acetic acid. The reaction solution was concentrated under reduced pressure. The resulting residue was purified by HPLC to afford Compound 357 (10 mg). EI-MS m/z: [M+H]⁺ 1461.43.

### Example 62: Preparation of Compound 359

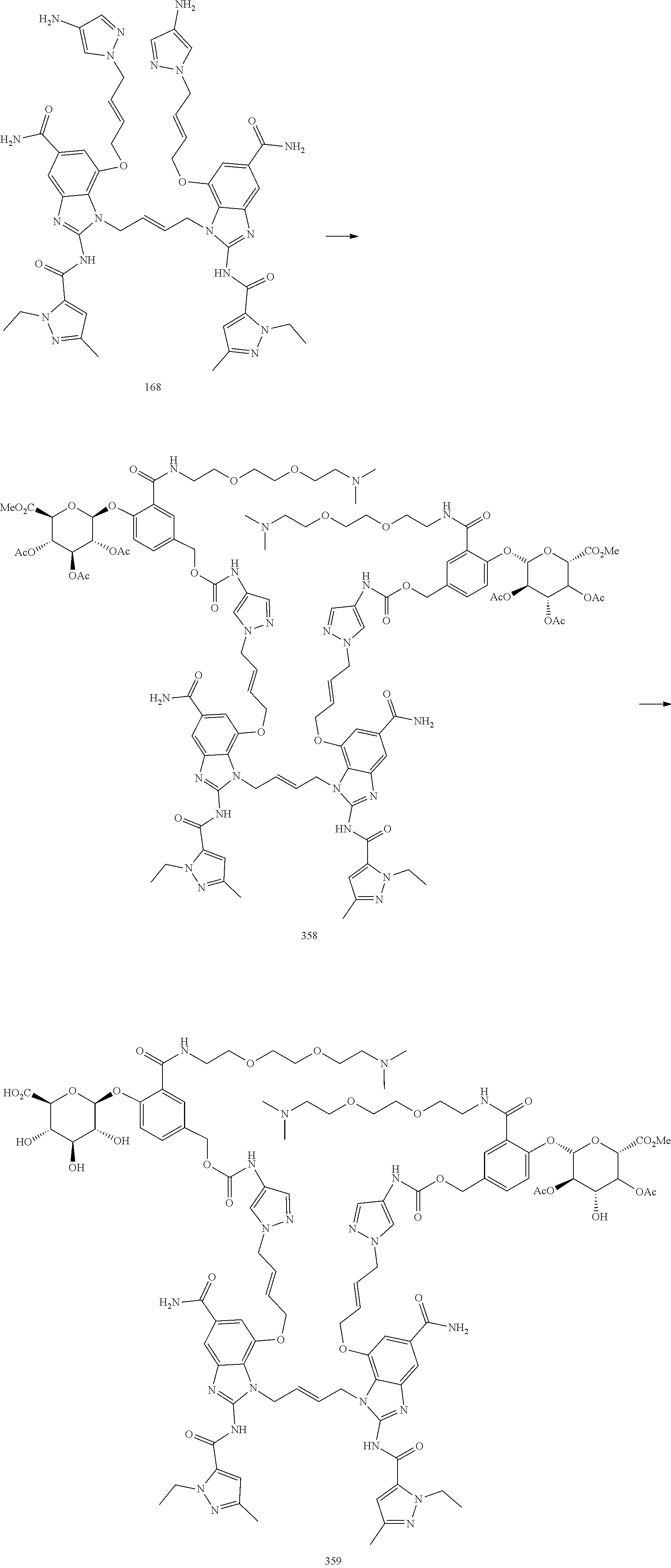

### Preparation of Intermediate Compound 358

To a solution of Intermediate Compound 168 (100 mg, 0.08 mmol) and Intermediate Compound 310 (152 mg, 0.19 mmol) in N,N-dimethylformamide (3 mL) were added 1-hydroxy-7-azabenzotriazole (HOAt, 4 mg, 0.03 mmol) and N,N′-diisopropylethylamine (0.07 mL, 0.38 mmol) at 0° C. under nitrogen. After stirred at room temperature for 17 hours, the reaction solution was concentrated under reduced pressure. The resulting residue was purified by reverse phase column chromatography to afford Intermediate Compound 358 (168 mg, 80%). EI-MS m/z: [1/2M+H]⁺ 1158.97, [1/3M+H]⁺ 772.95.

### Preparation of Compound 359

To a solution of Intermediate Compound 358 (168 mg, 0.073 mmol) in tetrahydrofuran (2 mL) and methanol (2 mL) was added lithium hydroxide monohydrate (92 mg, 2.18 mmol) in distilled water (3 mL) at −70° C. After stirred for 6 hours at room temperature, the reaction mixture was adjusted to pH 4-5 with acetic acid. The reaction solution was concentrated under reduced pressure. The resulting residue was purified by HPLC to afford Compound 359 (22 mg, 15%). EI-MS m/z: [M+H]⁺ 2036.20, [1/2M+H]⁺ 1018.64.

### Example 63: Preparation of Compound 361

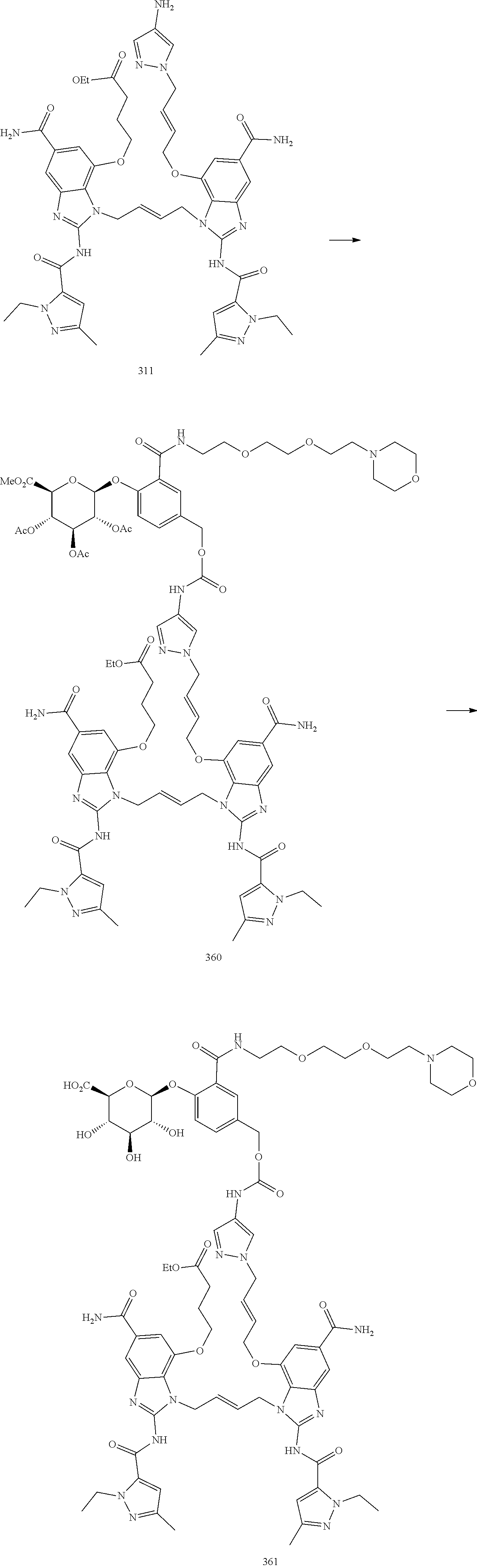

### Preparation of Intermediate Compound 360

To a solution of Intermediate Compound 311 (45 mg, 0.034 mmol) and Intermediate Compound 289 (31 mg, 0.034 mmol) in N,N-dimethylformamide (1 mL) was added N,N′-diisopropylethylamine (0.02 mL, 0.14 mmol) at 0° C. under nitrogen. After stirred at room temperature for 6 hours, the reaction solution was concentrated under reduced pressure to afford Intermediate Compound 360 (51 mg, crude), which was used without further purification. EI-MS m/z: [M+H]⁺ 1668.24.

### Preparation of Compound 361

To a solution of Intermediate Compound 360 (51 mg) in tetrahydrofuran (0.5 mL) and methanol (0.5 mL) was added lithium hydroxide monohydrate (12 mg, 0.28 mmol) in distilled water (0.5 mL) at −70° C. After stirred for 3 hours at 0° C., the reaction mixture was adjusted to pH 4-5 with acetic acid. The reaction solution was concentrated under reduced pressure. The resulting residue was purified by HPLC to afford Compound 361 (22 mg, 15%). EI-MS m/z: [M+H]⁺ 1500.26.

### Example 64: Preparation of Compound 363

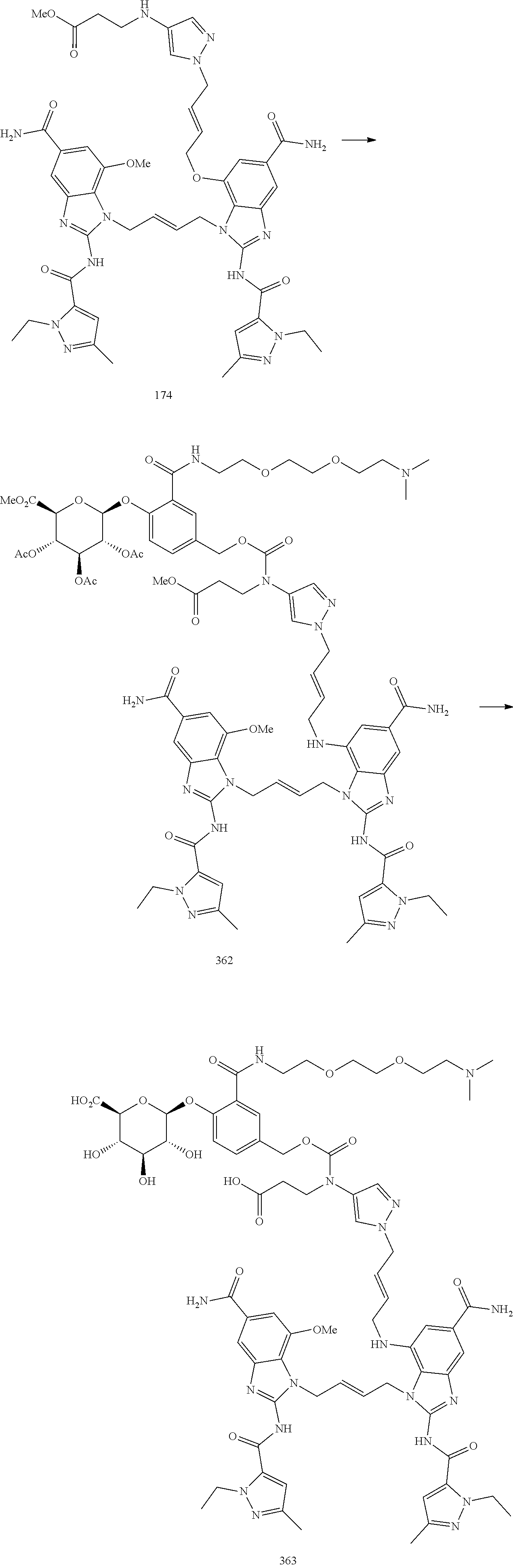

### Preparation of Intermediate Compound 362

To a solution of Intermediate Compound 174 (178 mg, 0.19 mmol) and Intermediate Compound 310 (183 mg, 0.23 mmol) in N,N-dimethylformamide (2 mL) were added 1-hydroxy-7-azabenzotriazole (HOAt, 5 mg, 0.04 mmol) and N,N′-diisopropylethylamine (0.13 mL, 0.94 mmol) at 0° C. under nitrogen. After stirred at room temperature for 40 hours under nitrogen, the reaction solution was diluted with ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 362 (65 mg, 21%). EI-MS m/z: [M/2+H]⁺807.37.

### Preparation of Compound 363

To a solution of Intermediate Compound 362 (72 mg, 0.05 mmol) in tetrahydrofuran (0.5 mL) and methanol (0.5 mL) was added lithium hydroxide monohydrate (9.4 mg, 0.22 mmol) in distilled water (1 mL) at −45° C. After stirred at 0° C. for 1 hours, the reaction solution was added lithium hydroxide monohydrate (20 mg, 0.48 mmol) in distilled water (5 mL) at 0° C. After stirred for 4 hours at room temperature, the reaction mixture was adjusted to pH 4-5 with acetic acid. The reaction solution was concentrated under reduced pressure. The resulting residue was purified by HPLC to afford Compound 363 (23 mg, 35%). EI-MS m/z: [M+H]⁺ 1458.92.

### Example 65: Preparation of Compound 367

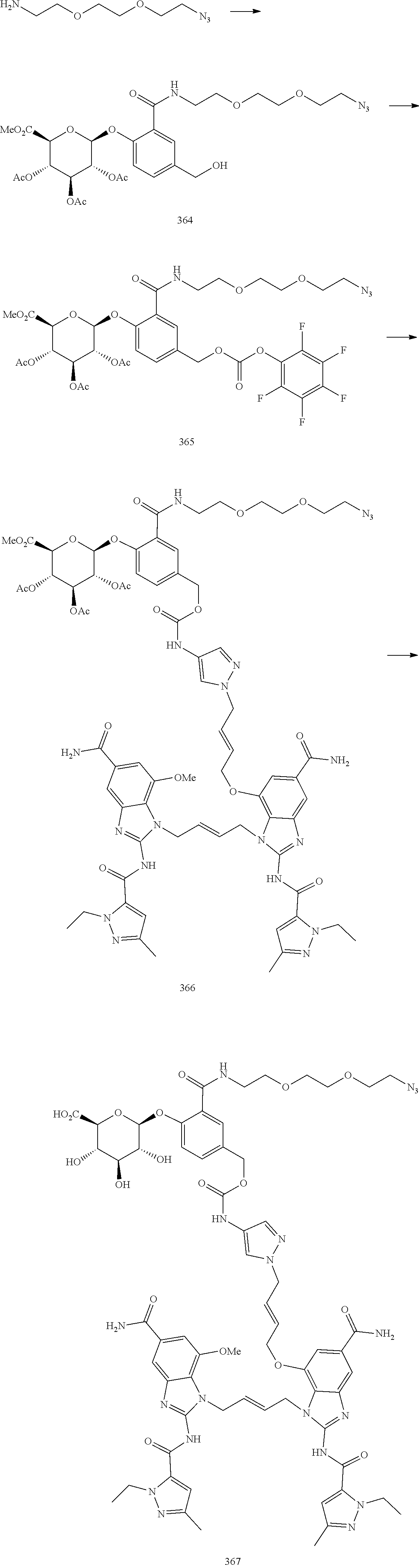

### Preparation of Intermediate Compound 364

To a solution of Intermediate Compound 205 (350 mg, 0.72 mmol, Intermediate Compound 205 was prepared by the method described in the International patent publication No. WO 2018/182341 A1) in N,N-dimethylformamide (2 mL) were added N-(3-dimethylaminopropyl)-N′-ethylcarbodiimide hydrochloride (EDC HCl, 411 mg, 1.08 mmol), 1-hydroxybenzotriazole (HOBt, 140 mg, 1.08 mmol), 2-(2-(2-azidoethoxy)ethoxy)ethanamine (151 mg, 0.86 mmol), and N,N′-diisopropylethylamine (0.23 mL, 2.16 mmol) at 0° C. under nitrogen. After stirred at room temperature for 15 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 364 (250 mg, 54%).

¹H-NMR (400 MHz, CDCl₃) δ 8.03 (m, 2H), 7.47 (m, 1H), 7.39 (m, 1H), 7.04 (m, 1H), 5.40 (m, 3H), 5.24 (s, 1H), 4.69 (s, 2H), 4.20 (m, 1H). 3.74 (m, 13H), 3.57 (m, 4H), 3.37 (m, 3H), 2.95 (s, 4H), 2.89 (s, 4H), 2.07 (s, 9H). EI-MS m/z: [M+H]⁺ 762.17.

### Preparation of Intermediate Compound 365

To a solution of Intermediate Compound 364 (250 mg, 0.39 mmol) in dichloromethane (2 mL) were added bis(pentafluorophenyl)carbonate (307 mg, 0.78 mmol) and N,N′-diisopropylethylamine (0.2 mL, 1.17 mmol) at 0° C. under nitrogen. After stirred at room temperature for 14 hours, the reaction solution was diluted with dichloromethane (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 365 (230 mg, 69%). EI-MS m/z: [M+H]⁺ 851.25.

### Preparation of Intermediate Compound 366

To a solution of Intermediate Compound 55 (223 mg, 0.26 mmol) and Intermediate Compound 365 (150 mg, 0.17 mmol) in N,N-dimethylformamide (3 mL) were added 1-hydroxy-7-azabenzotriazole (HOAt, 2.3 mg, 0.017 mmol) and N,N′-diisopropylethylamine (0.12 mL, 0.69 mmol) at ° C. under nitrogen. After stirred at room temperature for 3 hours, the reaction solution was concentrated under reduced pressure. The resulting residue was purified by column chromatography to afford Intermediate Compound 366 (240 mg, 90%). EI-MS m/z: [M/2+H]⁺763.14

### Preparation of Compound 367

To a solution of Intermediate Compound 366 (210 mg, 0.13 mmol) in tetrahydrofuran (1 mL) and methanol (1 mL) was added lithium hydroxide monohydrate (26 mg, 0.61 mmol) in distilled water (2 mL) at −50° C. After stirred for 3 hours at 0° C., the reaction mixture was adjusted to pH 4-5 with acetic acid. The reaction solution was concentrated under reduced pressure. The resulting residue was purified by HPLC to afford Compound 367 (80 mg, 41%).

¹H-NMR (400 MHz, DMSO) δ 9.50 (s, 1H), 8.20 (t, J=8.0 Hz, 1H), 7.92 (d, J=16.0 Hz, 2H), 7.95 (s, 1H), 7.83 (m, 3H), 7.64 (m, 1H), 7.30 (m, 4H), 7.20 (m, 1H), 6.50 (s, 2H), 5.92 (m, 1H), 5.79 (m, 2H), 5.69 (m, 2H), 5.42 (m, 2H), 5.12 (d, J=8.0 Hz, 1H), 2.05 (s, 2H), 4.90 (m, 4H), 3.97 (d, J=16.0 Hz, 1H), 3.69 (s, 3H). 3.61 (m, 8H), 2.68 (s, 2H), 2.33 (s, 2H), 2.09 (m, 6H), 1.27 (m, 8H). EI-MS m/z: [M/2+H]⁺693.12.

### Example 66: Preparation of Compound 372

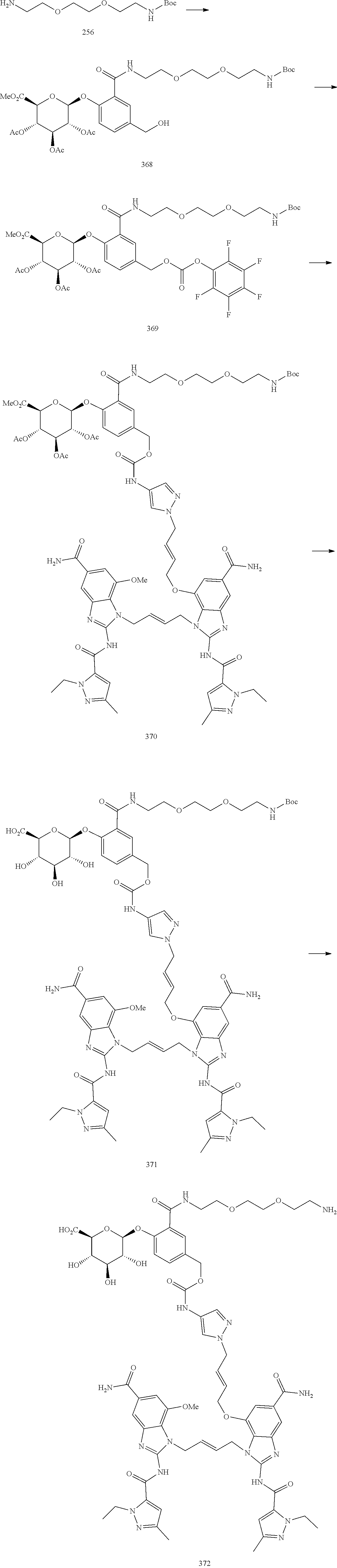

### Preparation of Intermediate Compound 368

To a solution of Intermediate Compound 205 (169 mg, 0.68 mmol, Intermediate Compound 205 was prepared by the method described in the International patent publication No. WO 2018/182341 A1) in N,N-dimethylformamide (5 mL) were added N-(3-dimethylaminopropyl)-N′-ethylcarbodiimide hydrochloride (EDC HCl, 178 mg, 0.92 mmol), 1-hydroxybenzotriazole (HOBt, 125 mg, 0.92 mmol), Intermediate Compound 256 (300 mg, 0.61 mmol), and N,N′-diisopropylethylamine (0.2 mL, 1.85 mmol) at 0° C. under nitrogen. After stirred at room temperature for 15 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 368 (390 mg, 88%).

¹H-NMR (400 MHz, CDCl₃) δ 7.89 (t, J=5.7 Hz, 1H), 7.59 (d, J=2.3 Hz, 1H), 7.39 (dd, J=8.5, 2.3 Hz, 1H), 7.12 (d, J=8.6 Hz, 1H), 6.74 (s, 1H), 5.77-5.70 (m, 1H), 5.49 (t, J=9.6 Hz, 1H), 5.20 (dd, J=9.7, 7.9 Hz, 2H), 5.07 (t, J=9.7 Hz, 1H), 4.73 (d, J=9.9 Hz, 2H), 4.46 (d, J=4.8 Hz, 2H), 3.63 (s, 1H), 3.59-3.48 (m, 6H), 3.43-3.37 (m, 4H), 3.06 (q, J=6.0 Hz, 2H), 2.04-1.96 (m, 9H), 1.37 (s, 10H). EI-MS m/z: [M+Na]⁺ 715.44.

### Preparation of Intermediate Compound 369

To a solution of Intermediate Compound 368 (150 mg, 0.20 mmol) in dichloromethane (3 mL) were added bis(pentafluorophenyl)carbonate (124 mg, 0.31 mmol) and N,N′-diisopropylethylamine (0.1 mL, 0.62 mmol) at 0° C. under nitrogen. After stirred at room temperature for 14 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 369 (180 mg, 92%).

¹H-NMR (400 MHz, DMSO) δ 7.98 (t, J=5.7 Hz, 1H), 7.74 (d, J=2.3 Hz, 1H), 7.59 (dd, J=8.6, 2.4 Hz, 1H), 7.22 (d, J=8.7 Hz, 1H), 6.73 (s, 1H), 5.84-5.73 (m, 3H), 5.49 (t, J=9.6 Hz, 1H), 5.39 (s, 2H), 5.22 (dd, J=9.7, 7.8 Hz, 1H), 5.08 (t, J=9.7 Hz, 1H), 4.75 (d, J=10.0 Hz, 1H), 3.64 (s, 3H), 3.53 (dt, J=10.8, 5.4 Hz, 6H), 3.39 (q, J=5.9 Hz, 6H), 3.06 (q, J=6.0 Hz, 2H), 2.69-2.65 (m, 1H), 2.35-2.30 (m, 1H), 1.36 (s, 9H). EI-MS m/z: [M+H]⁺ 925.42.

### Preparation of Intermediate Compound 370

To a solution of Intermediate Compound 55 (70 mg, 0.08 mmol) and Intermediate Compound 369 (90 mg, 0.10 mmol) in N,N-dimethylformamide (3 mL) were added 1-hydroxy-7-azabenzotriazole (HOAt, 1.1 mg, 0.01 mmol) and N,N′-diisopropylethylamine (0.07 mL, 0.40 mmol) at 0° C. under nitrogen. After stirred at room temperature for 3 hours, the reaction solution was concentrated under reduced pressure. The resulting residue was purified by column chromatography to afford Intermediate Compound 370 (120 mg, 91%).

¹H-NMR (400 MHz, DMSO) δ 12.82 (s, 2H), 9.49 (s, 1H), 8.48 (s, 3H), 7.92 (s, 3H), 7.63 (d, J=8.7 Hz, 3H), 7.46 (d, J=8.1 Hz, 1H), 7.31 (d, J=7.4 Hz, 4H), 7.15 (d, J=8.6 Hz, 1H), 6.72 (s, 1H), 6.50 (d, J=2.8 Hz, 2H), 5.85-5.63 (m, 4H), 5.48 (t, J=9.6 Hz, 1H), 5.21 (d, J=8.1 Hz, 1H), 5.12-5.00 (m, 3H), 4.89 (d, J=10.2 Hz, 3H), 4.72 (d, J=9.9 Hz, 1H), 4.52 (t, J=8.5 Hz, 6H), 3.69 (s, 2H), 3.62 (s, 7H), 3.56-3.47 (m, 5H), 3.20-3.11 (m, 6H), 3.05 (d, J=6.0 Hz, 5H), 2.09 (d, J=3.8 Hz, 4H), 2.05-1.98 (m, 6H), 1.36 (s, 9H). EI-MS m/z: [M/2+H]⁺ 801.00.

### Preparation of Intermediate Compound 371

To a solution of Intermediate Compound 370 (120 mg, 0.075 mmol) in tetrahydrofuran (1 mL) and methanol (1 mL) was added lithium hydroxide monohydrate (12 mg, 0.30 mmol) in distilled water (2 mL) at −50° C. After stirred for 3 hours at 0° C., the reaction mixture was adjusted to pH 4-5 with acetic acid. The reaction solution was concentrated under reduced pressure. The resulting residue was purified by HPLC to afford Intermediate Compound 371 (80 mg, 73%). EI-MS m/z: [M/2+H]⁺ 730.32.

### Preparation of Compound 372

To a solution of Intermediate 371 (80 mg, 0.075 mmol) in dichloromethane (3 mL) was added trifluoroacetic acid (0.45 mL) at 0° C. under nitrogen. After stirred at room temperature for 1.5 hours under nitrogen, the reaction solution was concentrated under reduced pressure. The resulting residue was purified by reverse phase column chromatography to afford Compound 372 (90 mg, 96%). EI-MS m/z: [M/2+H]⁺ 680.30.

### Example 67: Preparation of Compound 374

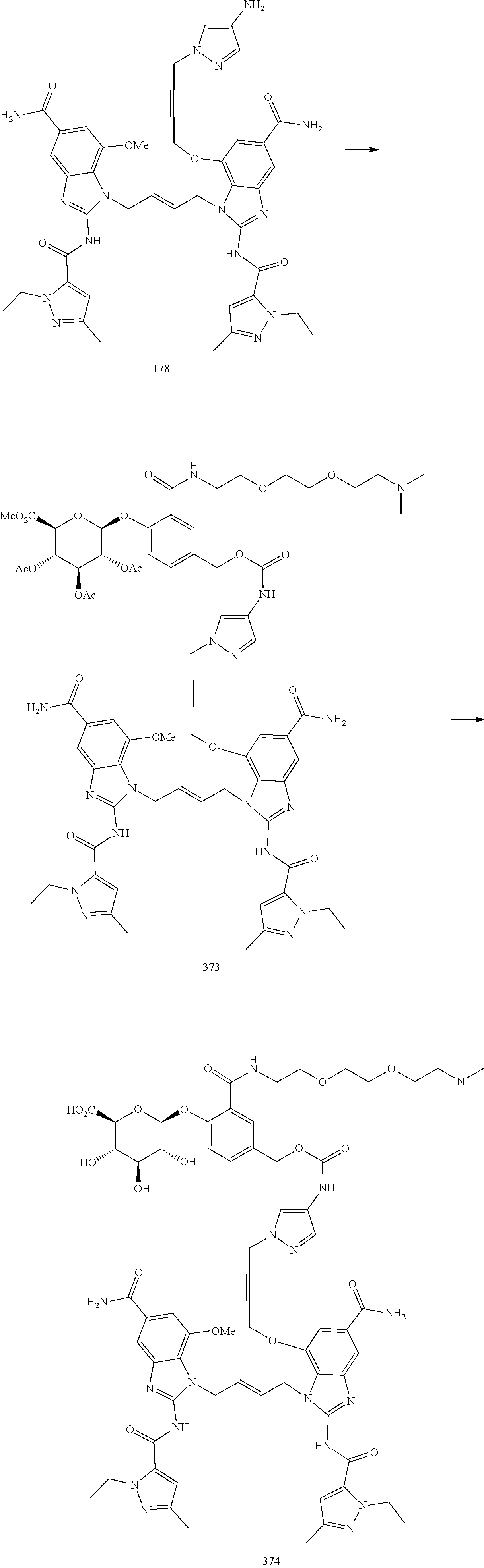

### Preparation of Intermediate Compound 373

To a solution of Intermediate Compound 259 (38 mg, 0.06 mmol) in dichloromethane (2 mL) were added N,N′-diisopropylethylamine (0.02 mL, 0.13 mmol) and triphosgene (8 mg, 0.03 mmol) at −70° C. under nitrogen. After stirred for 2 hours at −70° C. under nitrogen, the reaction solution was added Intermediate Compound 178 (40 mg, 0.03 mmol) and N,N′-diisopropylethylamine (0.023 mL, 0.17 mmol) in N,N-dimethylformamide (3 mL) at −70° C. After stirred at room temperature for 16 hours, the reaction solution was concentrated under reduced pressure to afford Intermediate Compound 373 (60 mg, crude), which was used without further purification. EI-MS m/z: [M+H]⁺ 1524.75.

### Preparation of Compound 374

To a solution of Intermediate Compound 373 (60 mg) in tetrahydrofuran (0.4 mL) and methanol (0.2 mL) was added lithium hydroxide monohydrate (20 mg, 0.49 mmol) in distilled water (1.2 mL) at −70° C. After stirred for 6 hours at 0° C., the reaction mixture was adjusted to pH 4-5 with acetic acid. The reaction solution was concentrated under reduced pressure. The resulting residue was purified by HPLC to afford Compound 374 (22 mg, 39%). EI-MS m/z: [M+H]⁺ 1384.70, [1/2M+H]⁺693.18.

### Example 68: Preparation of Compound 376

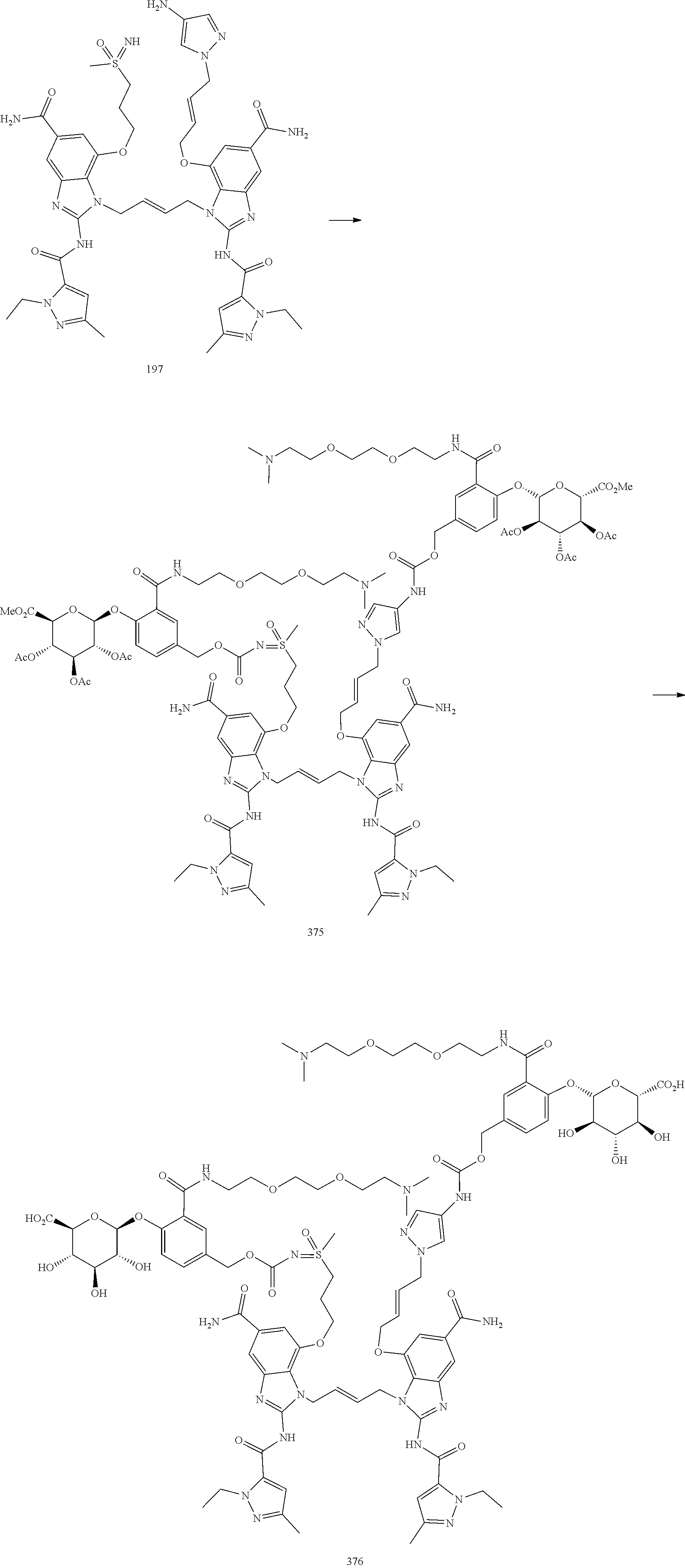

### Preparation of Intermediate Compound 375

To a solution of Intermediate Compound 259 (30 mg, 0.045 mmol) in dichloromethane (1 mL) were added N,N′-diisopropylethylamine (0.01 mL, 0.08 mmol) and triphosgene 5 mg, 0.017 mmol) at −70° C. under nitrogen. After stirred for 2 hours at −70° C., the reaction solution was added Intermediate Compound 197 (15 mg, 0.011 mmol) and N,N′-diisopropylethylamine (0.008 mL, 0.056 mmol) in N,N-dimethylformamide (3 mL) at −70° C. After stirred at room temperature for 16 hours under nitrogen, the reaction solution was concentrated under reduced pressure to afford Intermediate Compound 375 (40 mg, crude), which was used without further purification. EI-MS m/z: [1/2M+H]⁺ 1151.05, [1/3M+H]⁺ 767.81.

### Preparation of Compound 376

To a solution of Intermediate Compound 375 (40 mg) in tetrahydrofuran (0.4 mL) and methanol (0.2 mL) was added lithium hydroxide monohydrate (14 mg, 0.33 mmol) in distilled water (1.2 mL) at −70° C. After stirred for 6 hours at 0° C., the reaction mixture was adjusted to pH 4-5 with acetic acid. The reaction solution was concentrated under reduced pressure. The resulting residue was purified by HPLC to afford Compound 376 (5 mg, 19%). EI-MS m/z: [M+H]⁺ 2020.33, [1/2M+H]⁺1011.01, [1/3M+H]⁺674.39.

### Example 69: Preparation of Compound 381

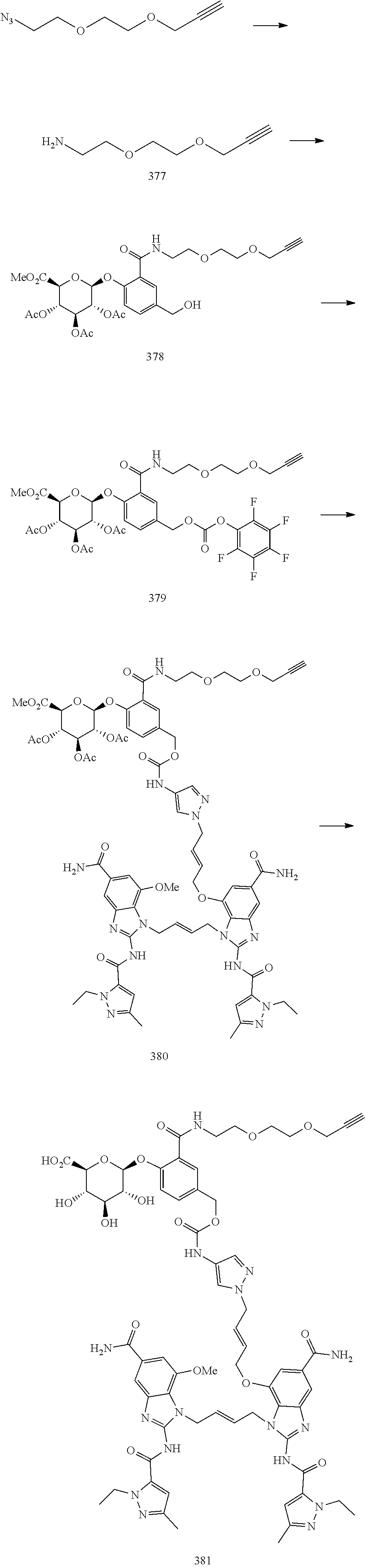

### Preparation of Intermediate Compound 377

To a solution of 3-(2-(2-azidoethoxy)ethoxy)prop-1-yne (260 mg, 1.53 mmol) in tetrahydrofuran (3 mL) were added triphenylphosphine (443 mg, 1.68 mmol) and distilled water (0.27 mL, 15.3 mmol) at 0° C. under nitrogen. The reaction mixture was refluxed for 16 hours. After the reaction was completed, the reaction solution was concentrated under reduced pressure. The resulting residue was purified by column chromatography to afford Intermediate Compound 377 (160 mg, 72%).

¹H-NMR (400 MHz, CDCl₃) δ 4.22 (s, 2H), 4.14-3.93 (m, 4H), 3.66-3.59 (m, 2H), 2.95-3.93 (m, 2H). EI-MS m/z: [M+H]⁺ 144.28.

### Preparation of Intermediate Compound 378

To a solution of Intermediate Compound 205 (141 mg, 0.99 mmol, Intermediate Compound 205 was prepared by the method described in the International patent publication No. WO 2018/182341 A1) in N,N-dimethylformamide (5 mL) were added N-(3-dimethylaminopropyl)-N′-ethylcarbodiimide hydrochloride (EDC HCl, 189 mg, 0.99 mmol), 1-hydroxybenzotriazole (HOBt, 133 mg, 0.99 mmol), Intermediate Compound 377 (320 mg, 0.66 mmol), and N,N′-diisopropylethylamine (0.21 mL, 1.98 mmol) at 0° C. under nitrogen. After stirred at room temperature for 15 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 378 (330 mg, 81%).

¹H-NMR (400 MHz, DMSO) δ 7.95 (s, 1H), 7.89 (t, J=5.6 Hz, 2H), 7.60 (d, J=2.3 Hz, 1H), 7.39 (dd, J=8.6, 2.3 Hz, 2H), 7.11 (d, J=8.5 Hz, 2H), 5.78-5.70 (m, 2H), 5.49 (t, J=9.6 Hz, 2H), 5.20 (dd, J=9.7, 7.9 Hz, 3H), 5.07 (t, J=9.8 Hz, 2H), 4.73 (d, J=9.9 Hz, 2H), 4.46 (s, 3H), 4.15 (d, J=2.4 Hz, 4H), 3.63 (s, 5H), 3.58 (s, 8H), 2.89 (s, 3H), 2.73 (s, 3H), 2.05-1.98 (m, 12H). EI-MS m/z: [M+H]⁺ 610.34.

### Preparation of Intermediate Compound 379

To a solution of Intermediate Compound 378 (110 mg, 0.18 mmol) in dichloromethane (3 mL) were added bis(pentafluorophenyl)carbonate (71 mg, 0.18 mmol) and N,N′-diisopropylethylamine (0.1 mL, 0.62 mmol) at 0° C. under nitrogen. After stirred at room temperature for 14 hours, the reaction solution was diluted with dichloromethane (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 379 (140 mg, 94%). EI-MS m/z: [M+H]⁺ 820.34.

### Preparation of Intermediate Compound 380

To a solution of Intermediate Compound 55 (44 mg, 0.05 mmol) and Intermediate Compound 379 (50 mg, 0.04 mmol) in N,N-dimethylformamide (2 mL) was added N,N′-diisopropylethylamine (0.036 mL, 0.20 mmol) at 0° C. under nitrogen. After stirred at room temperature for 3 hours, the reaction solution was concentrated under reduced pressure. The resulting residue was purified by column chromatography to afford Intermediate Compound 380 (62 mg, 99%). EI-MS m/z: [M/2+H]⁺ 747.67.

### Preparation of Compound 381

To a solution of Intermediate Compound 380 (60 mg, 0.04 mmol) in tetrahydrofuran (1 mL) and methanol (1 mL) was added lithium hydroxide monohydrate (7.5 mg, 0.18 mmol) in distilled water (2 mL) at −50° C. After stirred for 3 hours at 0° C., the reaction mixture was adjusted to pH 4-5 with acetic acid. The reaction solution was concentrated under reduced pressure. The resulting residue was purified by HPLC to afford Compound 381 (40 mg, 73%).

¹H-NMR (400 MHz, DMSO) δ 12.82 (s, 1H), 9.51 (s, 1H), 8.29 (s, 1H), 7.94 (d, J=15.6 Hz, 2H), 7.83 (d, J=2.3 Hz, 1H), 7.64 (d, J=6.5 Hz, 2H), 7.54-7.43 (m, 1H), 7.37-7.27 (m, 3H), 7.24 (d, J=8.6 Hz, 1H), 6.50 (s, 2H), 5.88 (s, 1H), 5.79 (s, 2H), 5.70 (s, 2H), 5.13 (d, J=7.3 Hz, 1H), 5.05 (s, 2H), 4.89 (d, J=10.7 Hz, 3H), 4.53 (d, J=9.5 Hz, 5H), 4.14 (d, J=2.4 Hz, 1H), 3.96 (d, J=9.5 Hz, 2H), 3.69 (s, 2H), 3.57 (s, 3H), 3.52 (d, J=6.1 Hz, 2H), 2.67 (p, J=1.9 Hz, 4H), 2.33 (p, J=1.9 Hz, 3H), 2.09 (d, J=3.8 Hz, 5H), 1.91 (s, 1H), 1.25 (q, J=6.8 Hz, 5H). EI-MS m/z: [M/2+H]⁺677.68.

### Example 70: Preparation of Compound 396

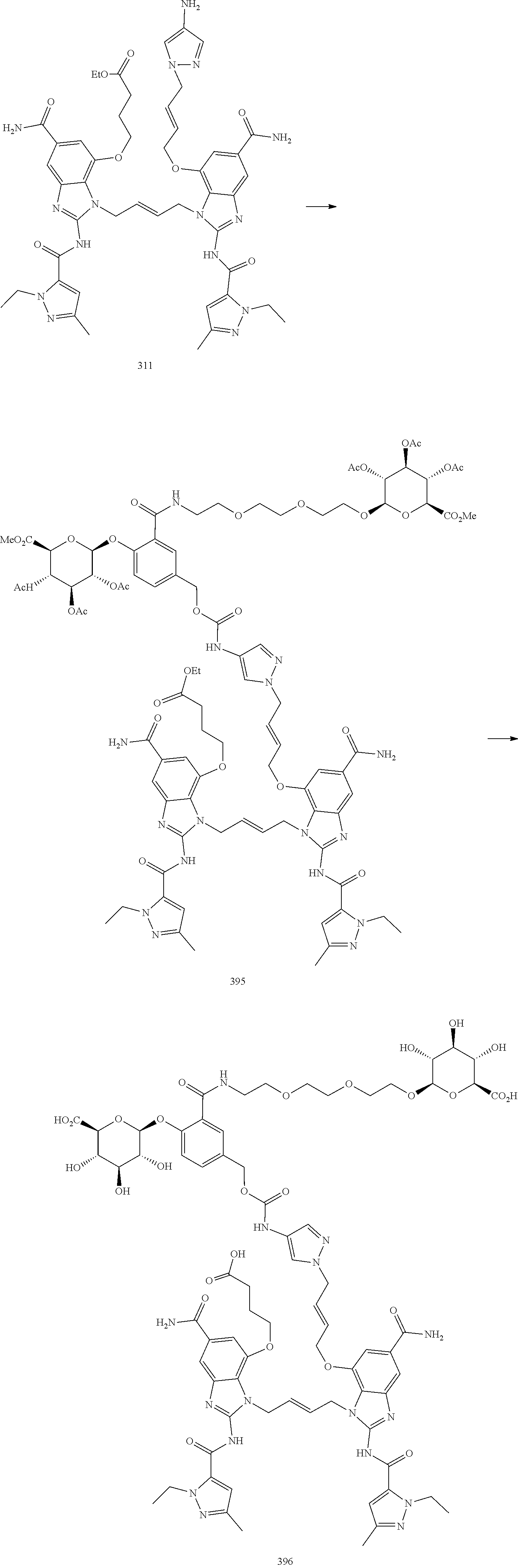

### Preparation of Intermediate Compound 395

To a solution of Intermediate Compound 311 (150 mg, 0.16 mmol) and Intermediate Compound 343 (150 mg, 0.13 mmol) in N,N-dimethylformamide (5 mL) were added 1-hydroxy-7-azabenzotriazole (HOAt, 3.6 mg, 0.026 mmol) and N,N′-diisopropylethylamine (0.11 mL, 0.65 mmol) at 0° C. under nitrogen. After stirred at room temperature for 13 hours, the reaction solution was diluted with ethyl acetate (50 mL) and washed with distilled water (50 mL×2). The organic layer was dried over anhydrous sodium sulfate and filtered. The filtrate was concentrated under reduced pressure. The resulting residue was purified by column chromatography to afford Intermediate Compound 395 (274 mg, 98%). EI-MS m/z: [M/2+H]⁺958.92.

### Preparation of Compound 396

To a solution of Intermediate Compound 395 (274 mg, 0.14 mmol) in tetrahydrofuran (2 mL) and methanol (2 mL) was added lithium hydroxide monohydrate (60 mg, 1.43 mmol) in distilled water (2 mL) at −45° C. After stirred for 0.5 hours at 0° C., the reaction solution was added lithium hydroxide monohydrate (60 mg, 1.43 mmol) in distilled water (12 mL) at 0° C. After stirred for 1 hours at room temperature, the reaction mixture was adjusted to pH 4-5 with acetic acid. The reaction solution was concentrated under reduced pressure. The resulting residue was purified by HPLC to afford Compound 396 (48 mg, 21%). EI-MS m/z: [M+H]⁺ 1608.92.

### Example 71: Preparation of Compound 400

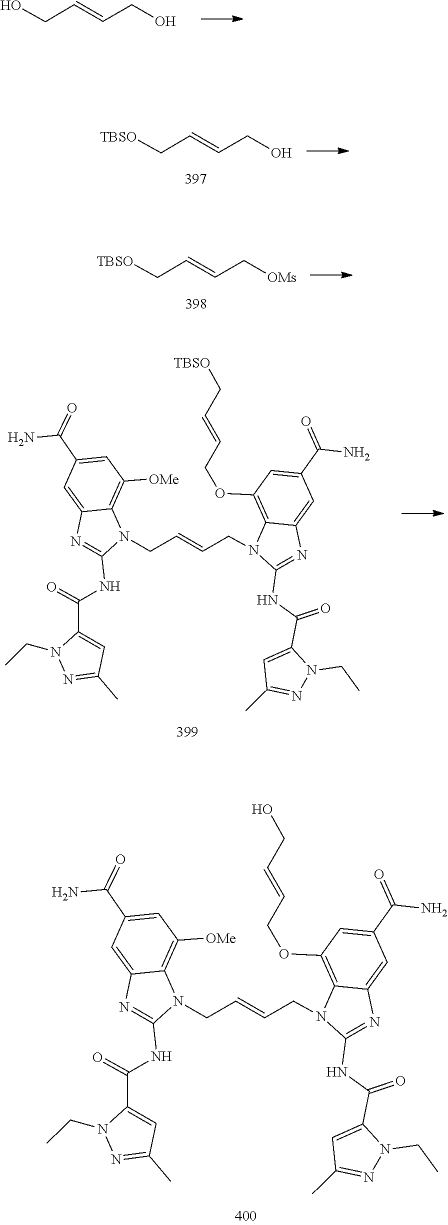

### Preparation of Intermediate Compound 397

To a solution of trans-2-butene-1,4-diol (1.5 g, 11.94 mmol) in dichloromethane (100 mL) were added trimethylamine (2.2 mL, 15.92 mmol) and t-butyldimethylsilyl chloride (1.0 g, 7.96 mmol) at 0° C. under nitrogen. After stirred at room temperature for 4 hours, the reaction solution was diluted with dichloromethane (100 mL) and washed with saturated aqueous ammonium chloride solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 397 (1.2 g, 74%).

¹H-NMR (400 MHz, MeOD) δ 5.88-5.71 (m, 2H), 4.19 (dt, J=4.3, 1.4 Hz, 2H), 4.09-4.02 (m, 2H), 0.92 (s, 9H).

### Preparation of Intermediate Compound 398

To a solution of Intermediate Compound 397 (200 mg, 0.99 mmol) in dichloromethane (10 mL) were added trimethylamine (0.28 mL, 1.98 mmol) and methanesulfonyl anhydride (206 mg, 1.19 mmol) at 0° C. under nitrogen. After stirred at room temperature for 2 hours, the reaction solution was diluted with dichloromethane (50 mL) and washed with distilled water (50 mL). The organic layer was dried over anhydrous sodium sulfate and filtered. The filtrate was concentrated under reduced pressure to afford Intermediate Compound 398 (140 mg, crude), which was used without further purification.

¹H-NMR (400 MHz, CDCl₃) δ 6.05-5.83 (m, 2H), 4.74 (dq, J=6.3, 1.2 Hz, 2H), 4.21 (dt, J=4.2, 1.6 Hz, 2H), 3.01 (s, 3H), 0.91 (s, 9H), 0.08 (s, 6H).

### Preparation of Intermediate Compound 399

To a solution of Intermediate Compound 66 (400 mg, 0.42 mmol, Intermediate Compound 66 was prepared by the method described in the International patent publication No. WO 2022/155518 A1) and Intermediate Compound 398 (130 mg, 0.46 mmol) in N,N-dimethylformamide (3 mL) was added cesium carbonate (685 mg, 2.10 mmol) were added at 0° C. under nitrogen. After stirred at room temperature for 3 hours, the reaction solution was concentrated under reduced pressure and diluted with dichloromethane (50 mL) and methanol (10 mL) and washed with distilled water (50 mL). The organic layer was dried over anhydrous sodium sulfate and filtered. The filtrate was concentrated under reduced pressure. The resulting residue was purified by column chromatography to afford Intermediate Compound 399 (128 mg, 33%). EI-MS m/z: [M+H]⁺ 907.58.

### Preparation of Compound 400

To a solution of Intermediate Compound 399 (20 mg, 0.02 mmol) in dichloromethane (0.4 mL) and methanol (0.2 mL) was added hydrochloric acid (4.0 M in 1,4-dioxane solution, 0.03 mL) at 0° C. under nitrogen. After stirred at room temperature for 2 hours, the reaction solution was concentrated under reduced pressure. The resulting residue was purified by HPLC to afford Compound 400 (10 mg, 42%). EI-MS m/z: [M+H]⁺ 794.27.

### Example 72: Preparation of Compound 408

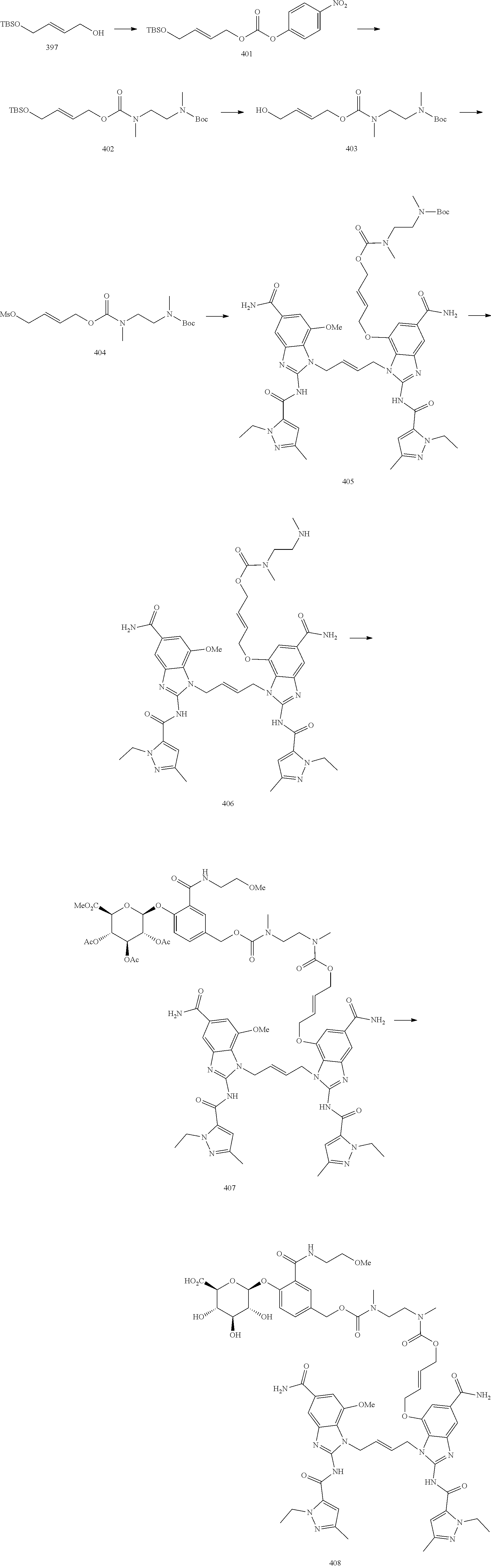

### Preparation of Intermediate Compound 401

To a solution of Intermediate Compound 397 (500 mg, 2.47 mmol) in dichloromethane (10 mL) were added trimethylamine (1.04 mL, 7.41 mmol), pyridine (0.60 mL, 7.41 mmol) and 4-nitrophenyl chloroformate (747 mg, 3.70 mmol) at 0° C. under nitrogen. After stirred at room temperature for 20 hours, the reaction solution was diluted with dichloromethane (100 mL) and washed with saturated aqueous sodium bicarbonate solution (50 mL) and 2% sodium hydroxide aqueous solution (50 mL). The organic layer was dried over anhydrous sodium sulfate and filtered. The filtrate was concentrated under reduced pressure. The resulting residue was purified by column chromatography to afford Intermediate Compound 401 (729 mg, 80%).

¹H-NMR (400 MHz, CDCl₃) δ 8.32-8.24 (m, 2H), 7.42-7.34 (m, 2H), 6.04-5.84 (m, 2H), 4.78 (dq, J=5.9, 1.1 Hz, 2H), 4.23 (dq, J=4.2, 1.4 Hz, 2H), 0.92 (s, 9H), 0.08 (s, 6H).

### Preparation of Intermediate Compound 402

To a solution of Intermediate Compound 401 (729 mg, 1.98 mmol) in dichloromethane (10 mL) were added trimethylamine (0.56 mL, 3.97 mmol) and tert-butyl methyl(2-(methylamino)ethyl)carbamate (485 mg, 2.58 mmol) at 0° C. under nitrogen. After stirred at room temperature for 3 hours, the reaction solution was diluted with dichloromethane (100 mL) and washed with saturated aqueous sodium bicarbonate solution (50 mL) and 2% sodium hydroxide aqueous solution (50 mL). The organic layer was dried over anhydrous sodium sulfate and filtered. The filtrate was concentrated under reduced pressure. The resulting residue was purified by column chromatography to afford Intermediate Compound 402 (825 mg, 99%).

¹H-NMR (400 MHz, CDCl₃) δ 5.85-5.78 (m, 2H), 4.58 (s, 2H), 4.18 (d, J=2.6 Hz, 2H), 3.37 (s, 4H), 2.94 (s, 3H), 2.88 (s, 3H), 1.45 (s, 9H), 0.91 (s, 9H), 0.07 (s, 6H).

### Preparation of Intermediate Compound 403

To a solution of Intermediate Compound 402 (825 mg, 0.43 mmol) in tetrahydrofuran (10 mL) was added tetrabutylammonium fluoride solution (1.0 M in tetrahydrofuran, 3 mL, 2.97 mmol) at 0° C. under nitrogen. After stirred at room temperature for 17 hours, the reaction solution was diluted with dichloromethane (100 mL) and washed with saturated aqueous sodium bicarbonate solution (50 mL) and saturated aqueous ammonium chloride solution (50 mL). The organic layer was dried over anhydrous sodium sulfate and filtered. The filtrate was concentrated under reduced pressure. The resulting residue was purified by column chromatography to afford Intermediate Compound 403 (514 mg, 86%).

¹H-NMR (400 MHz, CDCl₃) δ 5.96-5.73 (m, 2H), 4.58 (d, J=5.9 Hz, 2H), 4.15 (d, J=12.5 Hz, 2H), 3.45-3.27 (m, 4H), 2.94 (d, J=5.0 Hz, 3H), 2.87 (d, J=11.5 Hz, 3H), 1.45 (s, 9H).

### Preparation of Intermediate Compound 404

To a solution of Intermediate Compound 403 (150 mg, 0.50 mmol) in dichloromethane (5 mL) were added trimethylamine (0.14 mL, 0.99 mmol) and methanesulfonyl anhydride (103 mg, 0.59 mmol) at 0° C. under nitrogen. After stirred at room temperature for 2 hours, the reaction solution was diluted with dichloromethane (50 mL) and washed with distilled water (50 mL). The organic layer was dried over anhydrous sodium sulfate and filtered. The filtrate concentrated under reduced pressure to afford Intermediate Compound 404 (164 mg, crude), which was used without further purification.

¹H-NMR (400 MHz, CDCl₃) δ 6.06-5.77 (m, 2H), 4.73 (dt, J=6.2, 1.1 Hz, 2H), 4.63 (s, 2H), 3.38 (s, 4H), 3.03 (s, 3H), 2.95 (s, 3H), 2.87 (d, J=7.8 Hz, 3H), 1.45 (s, 9H).

### Preparation of Intermediate Compound 405

To a solution of Intermediate Compound 66 (350 mg, 0.37 mmol, Intermediate Compound 66 was prepared by the method described in the International patent publication No. WO 2022/155518 A1) and Intermediate Compound 404 (154 mg, 0.40 mmol) in N,N-dimethylformamide (2 mL) was added cesium carbonate (600 mg, 1.84 mmol) were added at 0° C. under nitrogen. After stirred at room temperature for 3 hours, the reaction solution was concentrated under reduced pressure and diluted with dichloromethane (50 mL) and methanol (10 mL) and washed with distilled water (30 mL). The organic layer was dried over anhydrous sodium sulfate and filtered. The filtrate was concentrated under reduced pressure. The resulting residue was purified by column chromatography to afford Intermediate Compound 405 (207 mg, 56%). EI-MS m/z: [M+H]⁺ 1007.74.

### Preparation of Intermediate Compound 406

To a solution of Intermediate 405 (75 mg, 0.12 mmol) in dichloromethane (1 mL) was added trifluoroacetic acid (0.2 mL) at 0° C. under nitrogen. After stirred at room temperature for 2 hours, the reaction solution was concentrated under reduced pressure to afford Intermediate Compound 406 (93 mg. crude), which was used without further purification. EI-MS m/z: [M+H]⁺ 908.38.

### Preparation of Intermediate Compound 407

To a solution of Intermediate Compound 204 (50 mg, 0.067 mmol) and Intermediate Compound 406 (93 mg) in N,N-dimethylformamide (2 mL) was added N,N′-diisopropylethylamine (0.039 mL, 0.280 mmol) at 0° C. under nitrogen. After stirred at room temperature for 19 hours, the reaction solution was concentrated under reduced pressure. The solid was triturated with dichloromethane and diethyl ether. The reaction mixture was filtered and dried to afford Intermediate Compound 407 (77 mg, 74%), which was used without further purification. EI-MS m/z: [M+H]⁺ 1475.87.

### Preparation of Compound 408

To a solution of Intermediate Compound 407 (77 mg) in tetrahydrofuran (1 mL) and methanol (0.5 mL) was added lithium hydroxide monohydrate (14 mg, 0.33 mmol) in distilled water (1.5 mL) at −70° C. After stirred for 4 hours at 0° C., the reaction mixture was adjusted to pH 4-5 with acetic acid. The reaction solution was concentrated under reduced pressure. The resulting residue was purified by HPLC to afford Compound 408 (42 mg, 75%). EI-MS m/z [M+H]⁺ 1335.77.

### Example 73: Preparation of Compound 415

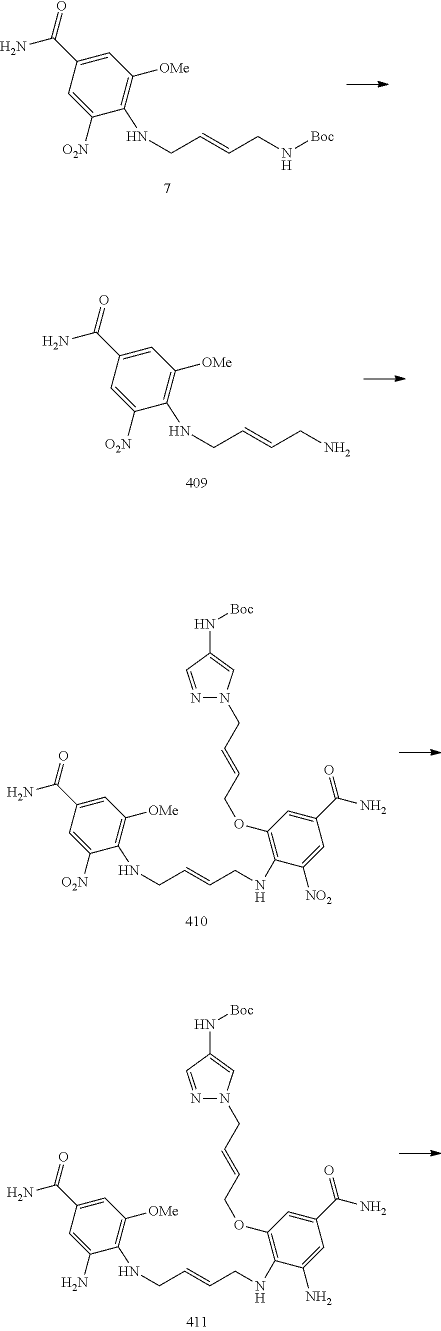 [SMILES: COc1cc(C(N)=O)cc([N+](=O)[O-])c1NC/C=C/CNC(=O)OC(C)(C)C ; COc1cc(C(N)=O)cc([N+](=O)[O-])c1NC/C=C/CN ; COc1cc(C(N)=O)cc([N+](=O)[O-])c1NC/C=C/CNc1c(OC/C=C/Cn2cc(NC(=O)OC(C)(C)C)cn2)cc(C(N)=O)cc1[N+](=O)[O-] ; COc1cc(C(N)=O)cc(N)c1NC/C=C/CNc1c(N)cc(C(N)=O)cc1OC/C=C/Cn1cc(NC(=O)OC(C)(C)C)cn1]

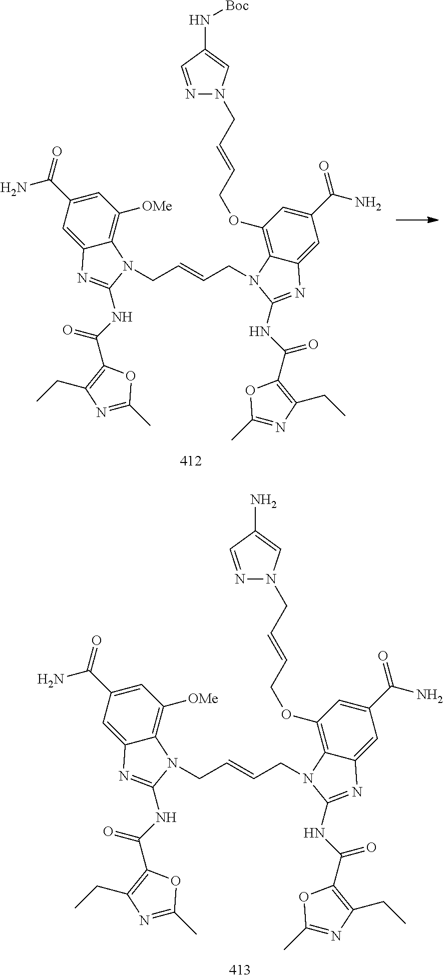 [SMILES: CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OC/C=C/Cn3cc(NC(=O)OC(C)(C)C)cn3)c21 ; CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OC/C=C/Cn3cc(N)cn3)c21]

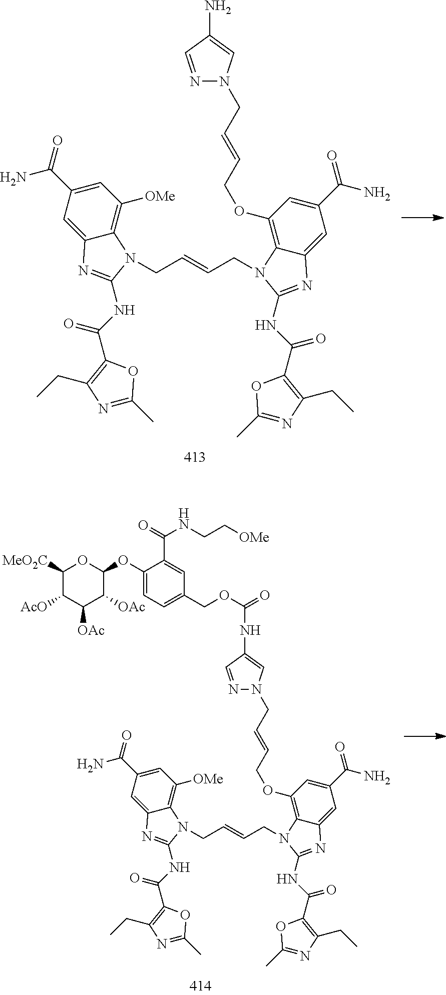 [SMILES: CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OC/C=C/Cn3cc(N)cn3)c21 ; CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OC/C=C/Cn3cc(NC(=O)OCc4ccc(O[C@@H]5O[C@H](C(=O)OC)[C@@H](OC(C)=O)[C@H](OC(C)=O)[C@H]5OC(C)=O)c(C(=O)NCCOC)c4)cn3)c21]

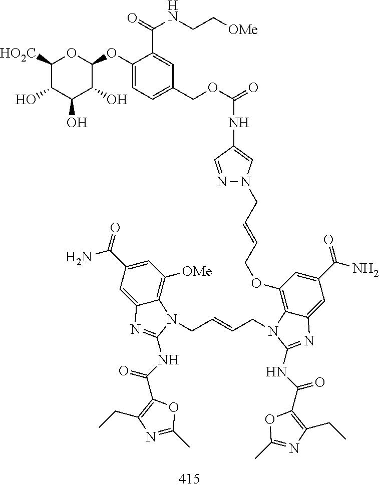 [SMILES: CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOC)c4)cn3)c21]

### Preparation of Intermediate Compound 409

To a solution of Intermediate Compound 7 (741 mg, 1.95 mmol) in methanol (2 mL) was added hydrochloric acid (4.0 M in 1,4-dioxane solution, 4.2 mL) at 0° C. under nitrogen. After stirred at room temperature for 2 hours, the reaction mixture was triturated with dichloromethane and diethyl ether. The reaction mixture was filtered and dried to afford Intermediate Compound 409 (614 mg, 89%), which was used without further purification. EI-MS m/z: [M+H]⁺ 281.41.

### Preparation of Intermediate Compound 410

To a solution of Intermediate Compound 409 (469 mg, 1.33 mmol) and Intermediate Compound 51 (300 mg, 0.66 mmol) in normal butyl alcohol (3 mL) was added triethylamine (0.65 mL, 4.65 mmol) at room temperature. After stirred 120° C. for 60 hours under sealed. The reaction solution was cooled to room temperature and concentrated under reduced pressure. The resulting residue was purified by column chromatography to afford Intermediate Compound 410 (140 mg, 30%). EI-MS m/z: [M+H]⁺ 697.06.

### Preparation of Intermediate Compound 411

To a solution of Intermediate Compound 410 (140 mg, 0.30 mmol) in methanol (4 mL) and distilled water (1 mL) were added aqueous ammonia solution (28˜30% ammonia, 0.4 mL) and sodium hydrosulfite (700 mg, 4.02 mmol). After stirred at room temperature for 2 hours, the reaction solution was diluted with methanol (10 mL) and filtered. The filtrate was concentrated under reduced pressure to afford Intermediate Compound 411 (120 mg, crude), which was used without further purification. EI-MS m/z: [M+H]⁺ 637.04.

### Preparation of Intermediate Compound 412

To a solution of Intermediate 411 (120 mg) in N,N-dimethylformamide (2 mL) was added compound 149 (148 mg, 0.75 mmol) in N,N-dimethylformamide (1 mL) at 0° C. under nitrogen. After stirred at room temperature for 1 hours, the reaction solution triethylamine (0.19 mL, 1.32 mmol) and N-(3-dimethylaminopropyl)-N-ethyl carbodiimide (144 mg, 0.75 mmol) were added at 0° C. After stirred at room temperature for 16 hours, the reaction solution was concentrated under reduced pressure. The resulting residue was purified by column chromatography to afford Intermediate Compound 412 (25 mg, 14%). EI-MS m/z: [M+H]⁺ 961.42.

### Preparation of Intermediate Compound 413

To a solution of Intermediate 412 (25 mg, 0.07 mmol) in dichloromethane (2 mL) was added trifluoroacetic acid (0.5 mL) at 0° C. under nitrogen. After stirred at room temperature for 2 hours, the reaction solution was concentrated under reduced pressure. The resulting residue was purified by HPLC to afford Intermediate Compound 413 (10 mg, 56%). EI-MS m/z: [M+H]⁺ 861.31.

### Preparation of Intermediate Compound 414

To a solution of Intermediate Compound 204 (5.5 mg, 0.007 mmol) and Intermediate Compound 413 (8 mg, 0.007 mmol) in N,N-dimethylformamide (0.3 mL) was added N,N′-diisopropylethylamine (0.006 mL, 0.033 mmol) at 0° C. under nitrogen. After stirred at room temperature for 3 hours, the reaction solution was concentrated under reduced pressure to afford Intermediate Compound 414 (9 mg, crude), which was used without further purification. EI-MS m/z: [M+H]⁺ 1428.79.

### Preparation of Compound 415

To a solution of Intermediate Compound 414 (9 mg) in tetrahydrofuran (0.2 mL) and methanol (0.1 mL) was added lithium hydroxide monohydrate (4 mg, 0.095 mmol) in distilled water (0.3 mL) at −70° C. After stirred for 4 hours at 0° C., the reaction mixture was adjusted to pH 4-5 with acetic acid. The reaction solution was concentrated under reduced pressure. The resulting residue was purified by HPLC to afford Compound 415 (6.8 mg, 71%). EI-MS m/z: [M+H]⁺ 1288.80.

### Example 74: Preparation of Compound 417

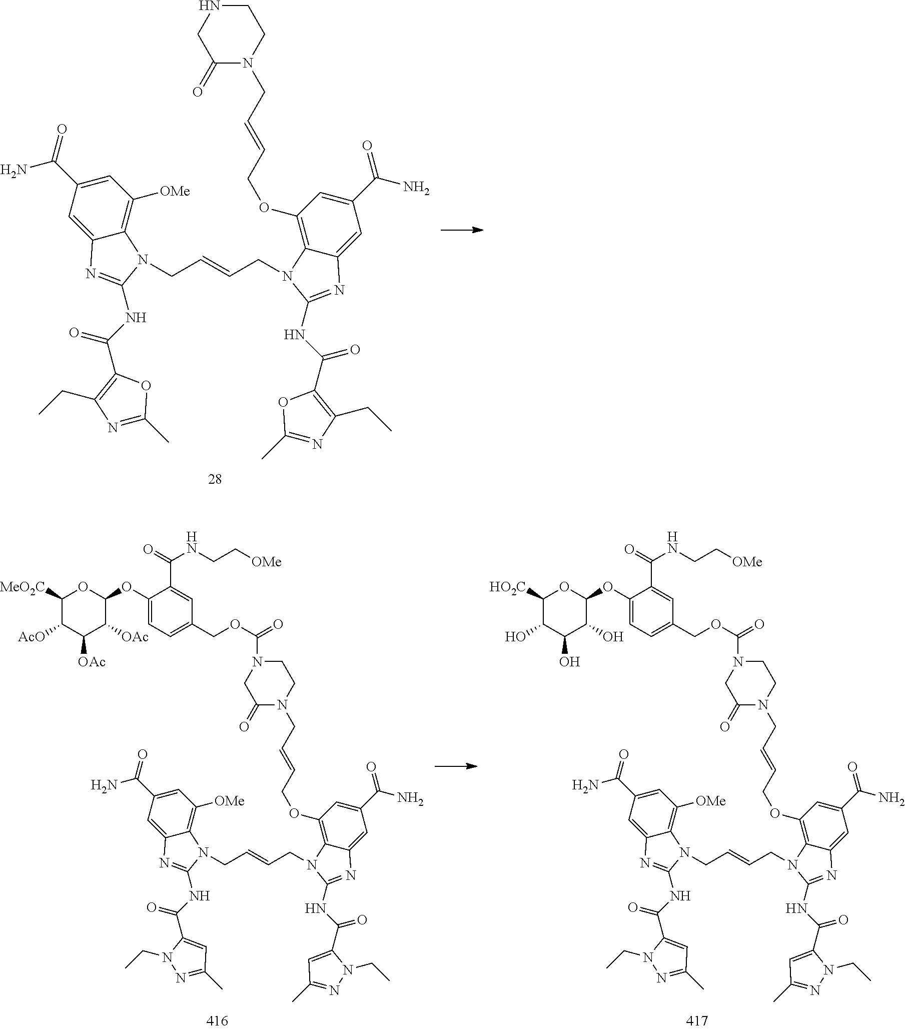

### Preparation of Intermediate Compound 416

To a solution of compound 28 (56 mg, 0.046 mmol) in N,N-dimethylformamide (2 mL) were added Intermediate Compound 204 (35 mg, 0.046 mmol), N,N′-diisopropylethylamine (0.064 mL, 0.370 mmol) and 1-hydroxy-7-azabenzotriazole (HOAt, 0.6 mg, 0.005 mmol) under nitrogen. After stirred at room temperature for 2 hours, the reaction solution was concentrated under reduced pressure to afford Intermediate Compound 416 (66 mg, crude), which was used without further purification. EI-MS m/z: [M+1/2]⁺722.10.

### Preparation of Compound 417

To a solution of Intermediate Compound 416 (66 mg) in methanol (0.5 mL) and tetrahydrofuran (0.5 mL) was added lithium hydroxide monohydrate (39 mg, 0.92 mmol) in distilled water (0.7 mL) at −50° C. under nitrogen. After stirred for 4 hours at 0° C., the reaction mixture was adjusted to pH 4-5 with acetic acid. The reaction solution was concentrated under reduced pressure. The resulting residue was purified by HPLC to afford Compound 417 (30 mg, 50%). EI-MS m/z: [M+1/2]⁺652.10, [M+H]⁺ 1302.65.

### Example 75: Preparation of Compound 419

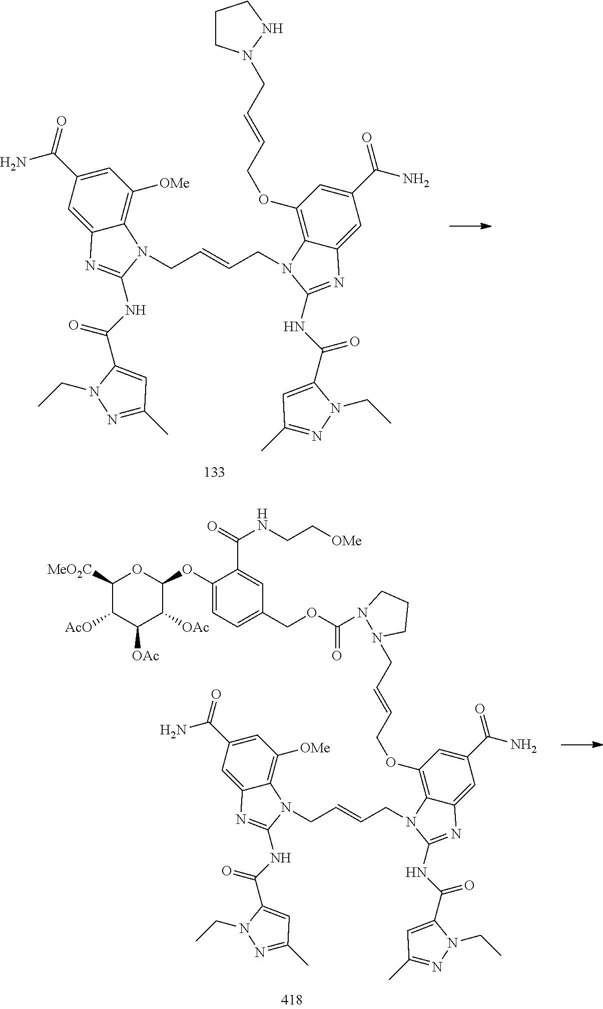 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)OC)[C@@H](OC(C)=O)[C@H](OC(C)=O)[C@H]4OC(C)=O)c(C(=O)NCCOC)c3)c21]

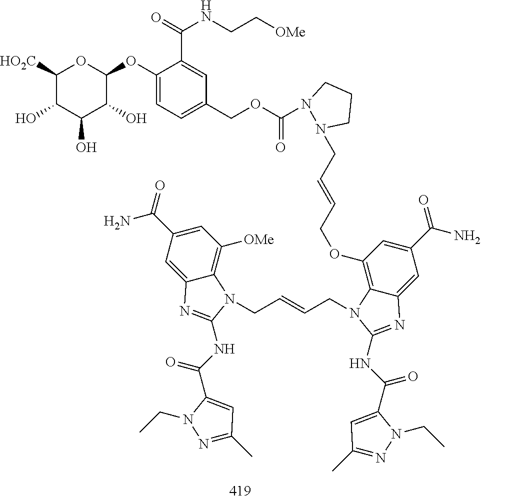 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOC)c3)c21]

### Preparation of Intermediate Compound 418

To a solution of compound 133 (30 mg, 0.025 mmol) in N,N-dimethylformamide (3 mL) were added Intermediate Compound 204 (22 mg, 0.030 mmol), N,N′-diisopropylethylamine (0.02 mL, 0.12 mmol) and 1-hydroxy-7-azabenzotriazole (HOAt, 1 mg, 0.01 mmol) under nitrogen. After stirred at room temperature for 2 hours, the reaction solution was concentrated under reduced pressure. The reaction mixture was added acetonitrile. The resulting solid was filtered and dried to afford Intermediate Compound 418 (35 mg, crude), which was used without further purification. EI-MS m/z: [M+1/2]⁺ 708.16, [M+H]⁺ 1414.67.

### Preparation of Compound 419

To a solution of Intermediate Compound 418 (35 mg) in methanol (1 mL) was added lithium hydroxide monohydrate (9 mg, 0.22 mmol) in distilled water (1 mL) at −50° C. under nitrogen. After stirred for 3 hours at 0° C., the reaction mixture was adjusted to pH 4-5 with acetic acid. The reaction solution was concentrated under reduced pressure. The resulting residue was purified by HPLC to afford Compound 419 (10 mg, 32%). EI-MS m/z: [M+1/2]⁺ 638.09, [M+H]⁺ 1274.53.

### Example 76: Preparation of Compound 422

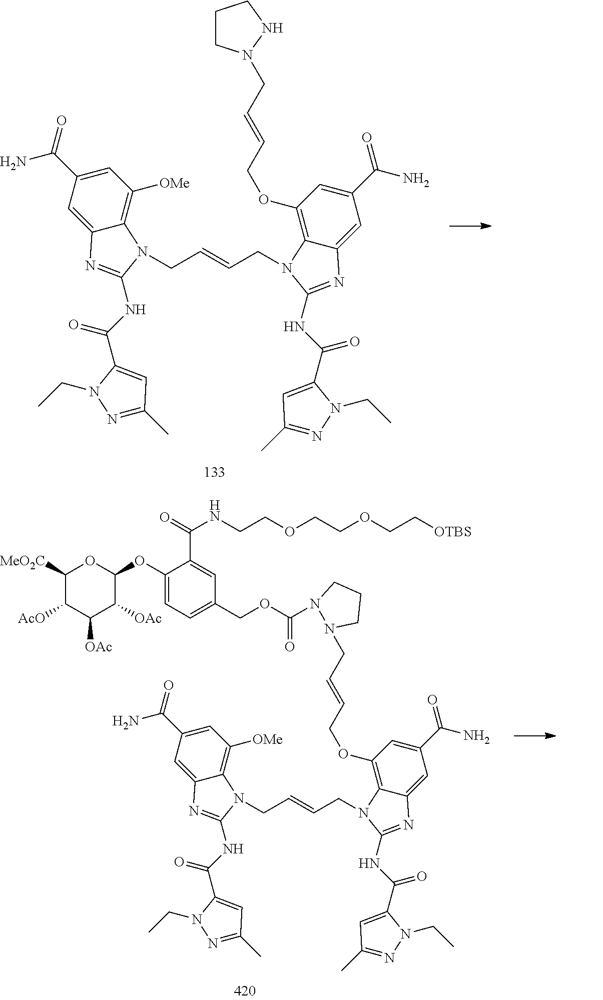 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)OC)[C@@H](OC(C)=O)[C@H](OC(C)=O)[C@H]4OC(C)=O)c(C(=O)NCCOCCOCCO[Si](C)(C)C(C)(C)C)c3)c21]

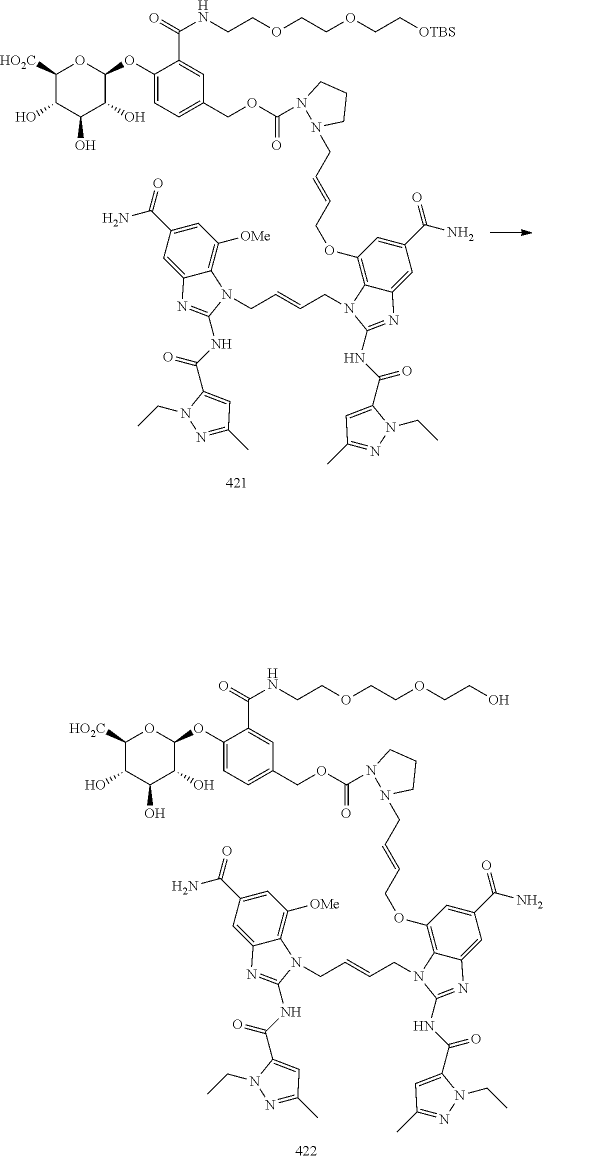 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOCCOCCO[Si](C)(C)C(C)(C)C)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOCCOCCO)c3)c21]

### Preparation of Intermediate Compound 420

To a solution of Intermediate Compound 267 (122 mg, 0.13 mmol) in N,N-dimethylformamide (1 mL) was added compound 133 (140 mg, 0.12 mmol), N,N-diisopropylethylamine (0.1 mL, 0.59 mmol) and HOAt (1-Hydroxy-7-azabenzotriazole, 3 mg, 0.02 mmol). After stirred at room temperature for 3 hours, the reaction mixture was concentrated under reduced pressure to afford Intermediate Compound 420 (189 mg, crude), which was used without further purification. EI-MS m/z: [M/2+H]⁺ 801.90.

### Preparation of Intermediate Compound 421

To a solution of Intermediate Compound 420 (189 mg, crude) in methanol (0.5 mL) and tetrahydrofuran (0.5 mL) was added lithium hydroxide monohydrate (49 mg, 1.17 mmol) in distilled water (0.5 mL) at −50° C. under nitrogen. After stirred at 0° C. for 3 hours, the reaction mixture was adjusted to pH 4-5 with acetic acid. The reaction mixture was concentrated under reduced pressure to afford Intermediate Compound 421 (172 mg, crude), which was used without further purification. EI-MS m/z: [M/2+H]⁺ 731.84.

### Preparation of Compound 422

To a solution of Intermediate Compound 421 (172 mg, crude) in dichloromethane (3 mL) was added trifluoroacetic acid (0.6 mL) at 0° C. under nitrogen. After stirred at room temperature for 2 hours, the reaction solution was concentrated under reduced pressure. The resulting residue was purified by HPLC to afford Compound 422 (70 mg, 38%). EI-MS m/z: [M+1/2]⁺674.80, [M+H]⁺ 1347.93.

### Example 77: Preparation of Compound 424

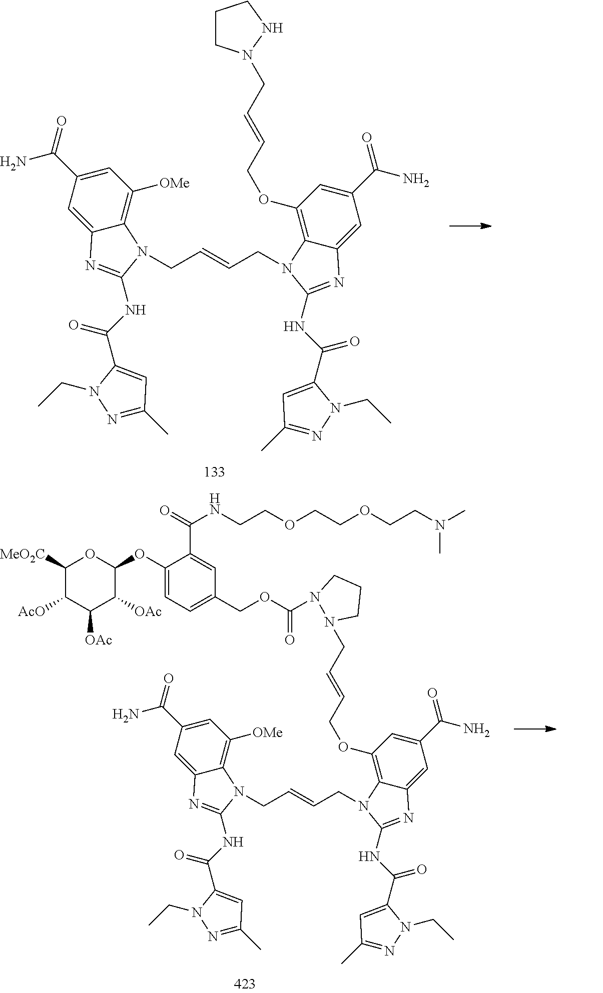 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)OC)[C@@H](OC(C)=O)[C@H](OC(C)=O)[C@H]4OC(C)=O)c(C(=O)NCCOCCOCCN(C)C)c3)c21]

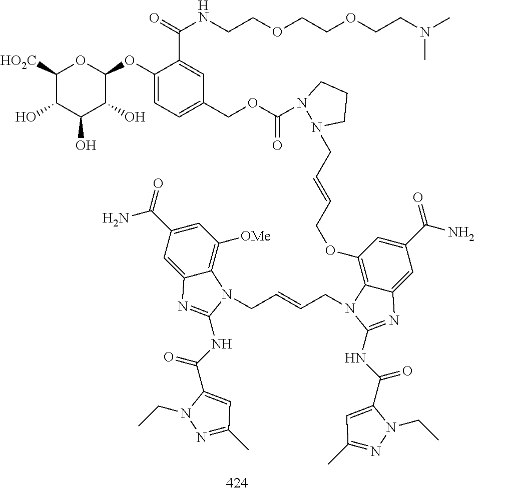 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOCCOCCN(C)C)c3)c21]

### Preparation of Intermediate Compound 423

To a solution of compound 133 (42 mg, 0.05 mmol) in N,N-dimethylformamide (2 mL) were added Intermediate Compound 260 (63 mg, 0.07 mmol), N,N′-diisopropylethylamine (0.04 mL, 0.25 mmol) and 1-hydroxy-7-azabenzotriazole (HOAt, 1 mg, 0.01 mmol) at 0° C. under nitrogen. After stirred at room temperature for 17 hours, the reaction solution was concentrated under reduced pressure. The resulting residue was purified by HPLC to afford Intermediate Compound 423 (40 mg, crude). EI-MS m/z: [M+1/2]⁺ 758.66, [M+H]⁺ 1515.77.

### Preparation of Compound 424

To a solution of Intermediate Compound 423 (40 mg) in tetrahydrofuran (0.75 mL) and methanol (0.75 mL) was added lithium hydroxide monohydrate (11 mg, 0.26 mmol) in distilled water (1 mL) at −40° C. under nitrogen. After stirred at 0° C. for 3 hours, the reaction mixture was adjusted to pH 4-5 with acetic acid. The reaction mixture was concentrated under reduced pressure. The resulting residue was purified by HPLC to afford Compound 424 (9.7 mg, 26%). EI-MS m/z: [M+1/2]⁺688.56, [M+H]⁺ 1376.62.

### Preparation Example 7: Preparation of Intermediate Compound 430

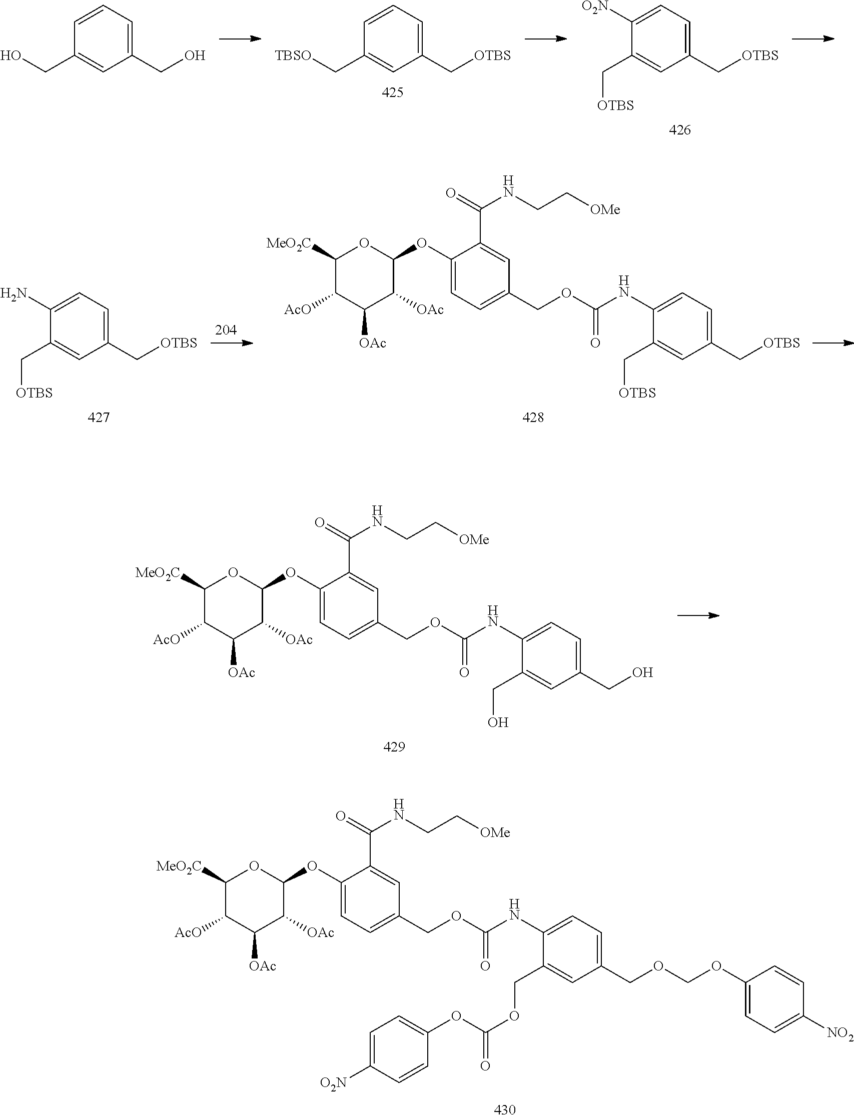

### Preparation of Intermediate Compound 425

To a solution of 1,3-benzenedimethanol (10.0 g, 72.4 mmol) in N,N-dimethylformamide (40 mL) were added imidazole (24.6 mg, 361.9 mmol) and tert-butyldimethylsilyl chloride (27.3 g, 180.9 mmol) at 0° C. under nitrogen. After stirred at room temperature for 16 hours, the reaction solution was diluted with ethyl acetate (100 mL) and washed with saturated aqueous ammonium chloride solution (70 mL) and distilled water (70 mL). The organic layer was dried over anhydrous sodium sulfate and filtered. The filtrate was concentrated under reduced pressure. The resulting residue was purified by column chromatography to afford Intermediate Compound 425 (21.5 g, 81%).

¹H-NMR (400 MHz, CDCl₃) δ 7.35-7.15 (m, 4H), 4.74 (s, 4H), 0.95 (s, 18H), 0.10 (s, 12H).

### Preparation of Intermediate Compound 426

To a solution of nitric acid (12.1 mL, 60%) in Acetic anhydride (100 mL) was added at 0° C. under nitrogen. After stirred at room temperature for 0.5 hours, the reaction solution Intermediate Compound 425 (21.5 g, 58.6 mmol) in acetic anhydride (45 mL) was dropwise at −20° C. After stirred at 0° C. for 0.5 hours, the reaction solution was diluted with ethyl acetate (100 mL) and washed with distilled water (80 mL), saturated aqueous sodium bicarbonate solution (70 mL×3) and saturated aqueous ammonium chloride solution (70 mL). The organic layer was dried over anhydrous sodium sulfate and filtered. The filtrate was concentrated under reduced pressure. The resulting residue was purified by column chromatography to afford Intermediate Compound 426 (13.1 g, 54%).

¹H-NMR (400 MHz, CDCl₃) δ 8.09 (d, J=8.4 Hz, 1H), s, 7.88 (s, 1H), 7.36 (d, J=6.0 Hz, 1H), 5.11 (s, 2H), 4.82 (s, 2H), 0.96 (d, J=7.2 Hz, 18H), 0.14 (d, J=8.8 Hz, 12H).

### Preparation of Intermediate Compound 427

To a solution of Intermediate Compound 426 (4.0 g, 9.7 mmol) in methanol (10 mL) and tetrahydrofuran (40 mL) were added palladium/charcoal (5% w/w, 200 mg) and ammonium formate (980 mg, 15.5 mmol). After stirred at room temperature under hydrogen balloon for 2 hours, the reaction solution was filtered through Celite and washed with dichloromethane (200 mL). The reaction solution was concentrated under reduced pressure. The resulting residue was purified by column chromatography to afford Intermediate Compound 427 (3.6 g, 96%).

¹H-NMR (400 MHz, CDCl₃) δ 7.02-6.99 (m, 2H), 6.63 (d, J=8.0 Hz, 1H), 4.70 (s, 2H), 4.61 (s, 2H), 0.90 (d, J=9.6 Hz, 18H), 0.07 (s, 12H). EI-MS m/z: [M+H]⁺ 382.42.

### Preparation of Intermediate Compound 428

To a solution of Intermediate Compound 204 (2.0 g, 2.66 mmol) and Intermediate Compound 427 (349 mg, 2.93 mmol) in N,N-dimethylformamide (10 mL) was added N,N′-diisopropylethylamine (1.4 mL, 8.00 mmol) at 0° C. under nitrogen. After stirred at room temperature for 19 hours, the reaction solution was diluted with ethyl acetate (40 mL) and washed with distilled water (30 mL×3). The organic layer was dried over anhydrous sodium sulfate and filtered. The filtrate was concentrated under reduced pressure. The resulting residue was purified by column chromatography to afford Intermediate Compound 428 (1.9 g, 77%).

¹H-NMR (400 MHz, CDCl₃) δ 8.33 (s, 1H), 8.06 (d, J=2.3 Hz, 1H), 7.96 (s, 1H), 7.50 (dd, J=8.5, 2.4 Hz, 1H), 7.45 (d, J=6.8 Hz, 1H), 7.21 (dd, J=8.4, 2.0 Hz, 1H), 7.05-7.02 (m, 2H), 5.44-5.31 (m, 3H), 5.31-5.25 (m, 1H), 5.17 (s, 2H), 4.71 (s, 2H), 4.66 (s, 2H), 4.19 (d, J=8.7 Hz, 1H), 3.78-3.68 (m, 4H), 3.62-3.52 (m, 3H), 3.42 (s, 3H), 2.06 (s, 9H), 0.90 (d, J=18.4 Hz, 18H), 0.07 (d, J=3.0 Hz, 12H).

### Preparation of Intermediate Compound 429

To a solution of Intermediate Compound 428 (1.94 g, 2.05 mmol) in methanol (40 mL) was added (1S)-(+)-10-camphorsulfonic acid (190 mg, 0.82 mmol) at 0° C. under nitrogen. After stirred for 2 hours at 0° C., the reaction solution was neutralized with triethylamine. After concentration reduced pressure, the resulting residue was purified by column chromatography to afford Intermediate Compound 429 (1.41 g, 95%).

¹H-NMR (400 MHz, CDCl₃) δ 8.08 (d, J=1.6 Hz, 1H), 8.05-7.85 (m, 2H), 7.53-7.44 (m, 2H), 7.29 (dd, J=8.1, 2.1 Hz, 1H), 7.19 (s, 1H), 7.04 (d, J=8.5 Hz, 1H), 5.44-5.24 (m, 2H), 5.17 (s, 2H), 4.70 (d, J=4.4 Hz, 2H), 4.63 (d, J=4.2 Hz, 2H), 4.19 (d, J=9.0 Hz, 1H), 3.74-3.65 (m, 4H), 3.62-3.50 (m, 3H), 3.42 (s, 3H), 2.38-2.32 (m, 1H), 2.06 (s, 9H), 1.76 (s, 1H).

### Preparation of Compound 430

To a solution of Intermediate Compound 429 (590 mg, 0.82 mmol) in dichloromethane (10 mL) were added pyridine (0.4 mL, 4.91 mmol) and 4-nitrophenyl chloroformate (825 mg, 4.09 mmol) at 0° C. under nitrogen. After stirred at room temperature for 16 hours, the reaction solution was diluted with ethyl acetate (40 mL) and washed with distilled water (30 mL). The organic layer was dried over anhydrous sodium sulfate and filtered. The filtrate was concentrated under reduced pressure. The resulting residue was purified by column chromatography to afford Compound 430 (819 mg, 95%).

¹H-NMR (400 MHz, CDCl₃) δ 8.27 (dd, J=9.2, 3.6 Hz, 4H), 8.14 (d, J=2.3 Hz, 1H), 7.93 (d, J=8.2 Hz, 1H), 7.54-7.45 (m, 5H), 7.41-7.33 (m, 4H), 7.05 (d, J=8.5 Hz, 1H), 5.45-5.31 (m, 6H), 5.27 (s, 2H), 5.20 (s, 2H), 4.19 (d, J=9.0 Hz, 1H), 3.75-3.71 (m, 4H), 3.63-3.52 (m, 3H), 3.42 (s, 3H), 2.08-2.02 (m, 9H). EI-MS m/z: [M+H]⁺ 1050.81.

### Example 78: Preparation of Compound 432

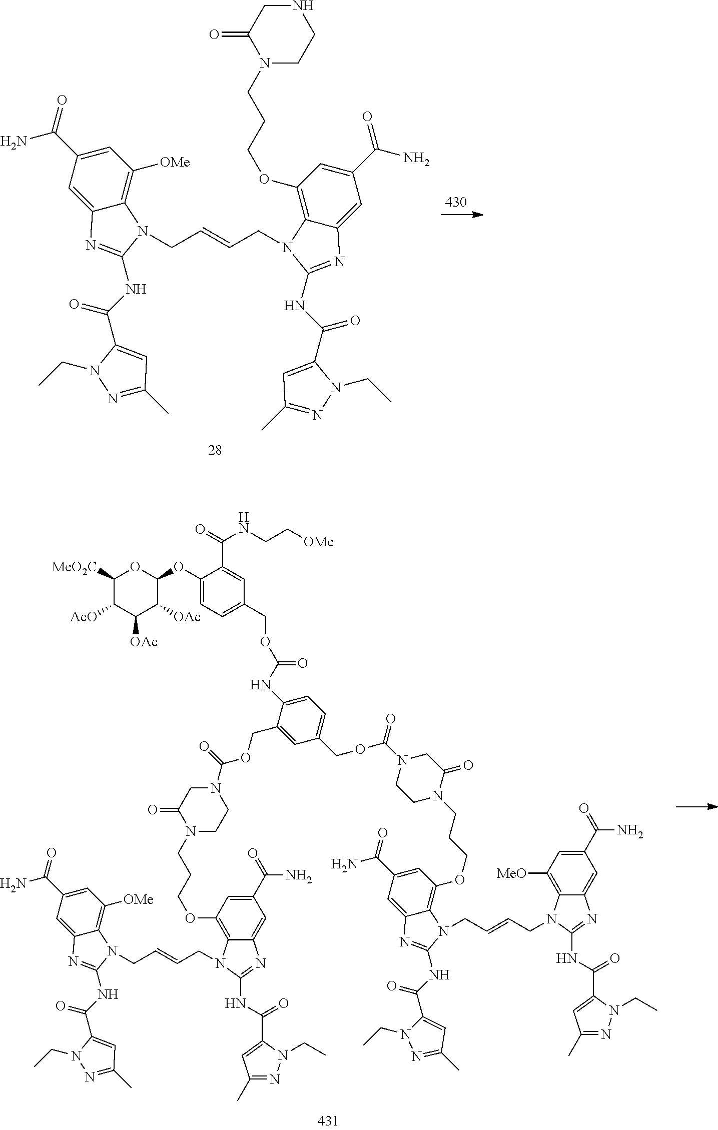 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCNCC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)OCc5ccc(O[C@@H]6O[C@H](C(=O)OC)[C@@H](OC(C)=O)[C@H](OC(C)=O)[C@H]6OC(C)=O)c(C(=O)NCCOC)c5)c(COC(=O)N5CCN(CCCOc6cc(C(N)=O)cc7nc(NC(=O)c8cc(C)nn8CC)n(C/C=C/Cn8c(NC(=O)c9cc(C)nn9CC)nc9cc(C(N)=O)cc(OC)c98)c67)C(=O)C5)c4)CC3=O)c21]

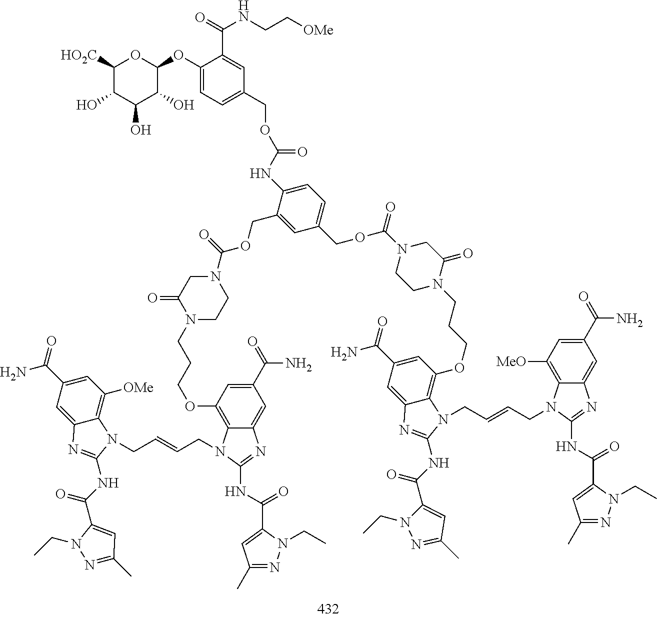 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)OCc5ccc(O[C@@H]6O[C@H](C(=O)O)[C@@H](O)[C@H](O)[C@H]6O)c(C(=O)NCCOC)c5)c(COC(=O)N5CCN(CCCOc6cc(C(N)=O)cc7nc(NC(=O)c8cc(C)nn8CC)n(C/C=C/Cn8c(NC(=O)c9cc(C)nn9CC)nc9cc(C(N)=O)cc(OC)c98)c67)C(=O)C5)c4)CC3=O)c21]

### Preparation of Intermediate Compound 431

To a solution of compound 28 (43 mg, 0.036 mmol) and Intermediate Compound 430 (18 mg, 0.017 mmol) in N,N-dimethylformamide (2 mL) were added 1-hydroxy-7-azabenzotriazole (HOAt, 0.46 mg, 0.003 mmol) and N,N′-diisopropylethylamine (0.024 mL, 0.137 mmol) at 0° C. under nitrogen. After stirred at room temperature for 19 hours, the reaction mixture was concentrated under reduced pressure. The resulting solid was washed with acetonitrile and dried to afford Intermediate Compound 431 (16 mg, crude), which was used without further purification. EI-MS m/z: [M+H]⁺ 2499.57, [M/2+H]⁺ 1250.63.

### Preparation of Compound 432

To a solution of Intermediate Compound 431 (16 mg) in methanol (0.5 mL) was added lithium hydroxide monohydrate (1.1 mg, 0.025 mmol) in distilled water (0.5 mL) at −50° C. After stirred for 3 hours at 0° C., the reaction mixture was adjusted to pH 4-5 with acetic acid. The reaction solution was concentrated under reduced pressure. The resulting residue was purified by HPLC to afford Compound 432 (3.4 mg, 22%). EI-MS m/z: [M+H]⁺ 2359.62, [M/2+H]⁺1180.70.

### Example 79: Preparation of Compound 436

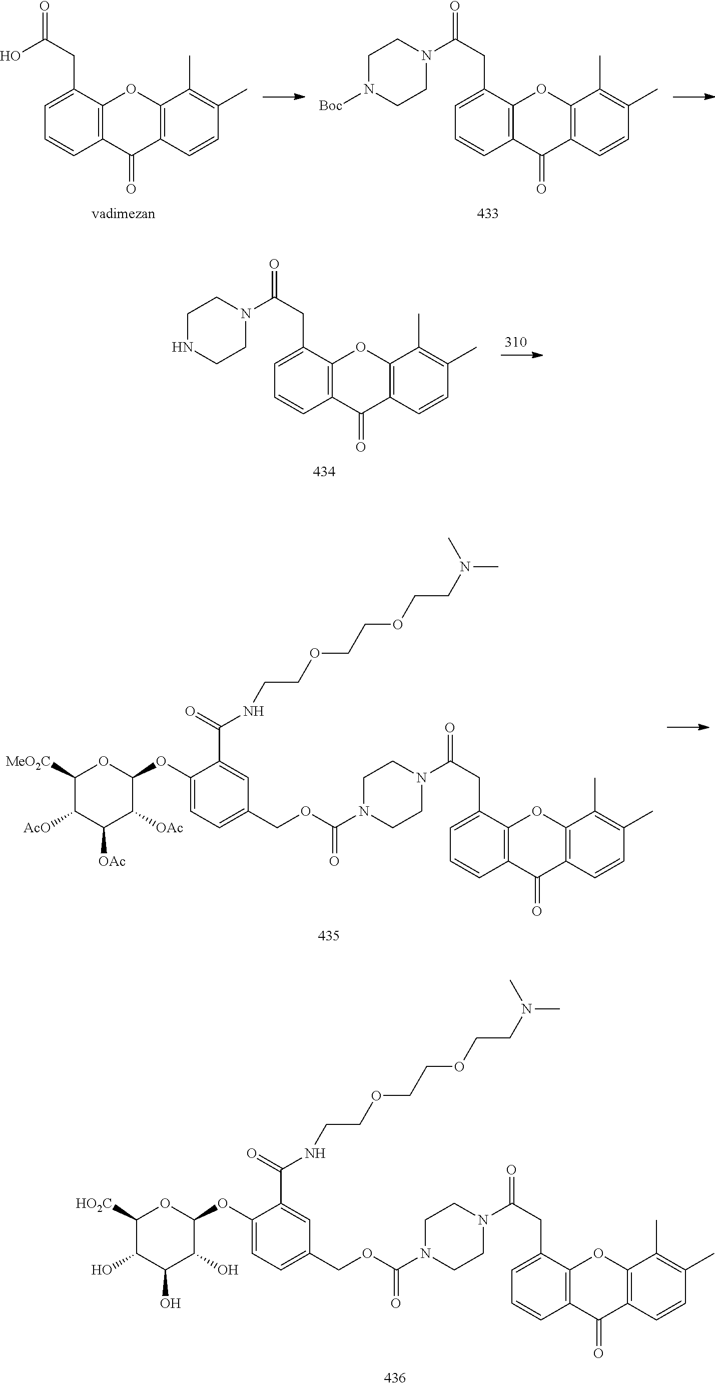 [SMILES: Cc1ccc2c(=O)c3cccc(CC(=O)O)c3oc2c1C ; Cc1ccc2c(=O)c3cccc(CC(=O)N4CCN(C(=O)OC(C)(C)C)CC4)c3oc2c1C ; Cc1ccc2c(=O)c3cccc(CC(=O)N4CCNCC4)c3oc2c1C ; COC(=O)[C@H]1O[C@@H](Oc2ccc(COC(=O)N3CCN(C(=O)Cc4cccc5c(=O)c6ccc(C)c(C)c6oc45)CC3)cc2C(=O)NCCOCCOCCN(C)C)[C@H](OC(C)=O)[C@@H](OC(C)=O)[C@@H]1OC(C)=O ; Cc1ccc2c(=O)c3cccc(CC(=O)N4CCN(C(=O)OCc5ccc(O[C@@H]6O[C@H](C(=O)O)[C@@H](O)[C@H](O)[C@H]6O)c(C(=O)NCCOCCOCCN(C)C)c5)CC4)c3oc2c1C]

### Preparation of Intermediate Compound 433

To a solution of vadimezan (100 mg, 0.35 mmol) in N,N-dimethylformamide (2 mL) were added N,N′-dicyclohexylcarbodiimide (DCC, 80 mg, 0.39 mmol), 1-hydroxybenzotriazole (HOBt, 53 mg, 0.39 mmol), 1-(tert-butoxycarbonyl)piperazine (72 mg, 0.39 mmol) and triethylamine (0.15 mL, 1.06 mmol) at 0° C. under nitrogen. After stirred at room temperature for 15 hours, the reaction solution was diluted with ethyl acetate (15 mL) and washed with distilled water (15 mL×2). The organic layer was dried over anhydrous sodium sulfate and filtered. The filtrate was concentrated under reduced pressure. The resulting residue was purified by column chromatography to afford Intermediate Compound 433 (53 mg, 33%). EI-MS m/z: [M+H]⁺ 451.136.

### Preparation of Intermediate Compound 434

To a solution of Intermediate 433 (53 mg, 0.12 mmol) in dichloromethane (1 mL) was added trifluoroacetic acid (0.6 mL) at 0° C. under nitrogen. After stirred at room temperature for 0.5 hours under nitrogen, the reaction solution was concentrated under reduced pressure to afford Intermediate Compound 434 (54 mg, crude), which was used without further purification. EI-MS m/z: [M+H]⁺ 351.148.

### Preparation of Intermediate Compound 435

To a solution of Intermediate Compound 434 (54 mg, 0.11 mmol) and Intermediate Compound 310 (90 mg, 0.11 mmol) in N,N-dimethylformamide (3 mL) were added 1-hydroxy-7-azabenzotriazole (HOAt, 3.2 mg, 0.02 mmol) and N,N′-diisopropylethylamine (0.1 mL, 0.59 mmol) at 0° C. under nitrogen. After stirred at room temperature for 19 hours, the reaction solution was concentrated under reduced pressure to afford Intermediate Compound 435 (113 mg), which was used without further purification. EI-MS m/z: [M+H]⁺ 1019.74.

### Preparation of Compound 436

To a solution of Intermediate Compound 435 (113 mg) in tetrahydrofuran (1 mL) and methanol (1 mL) was added lithium hydroxide monohydrate (23.4 mg, 0.56 mmol) in distilled water (1 mL) at −45° C. After stirred for 4 hours at 0° C., the reaction mixture was adjusted to pH 4-5 with acetic acid. The reaction solution was concentrated under reduced pressure. The resulting residue was purified by HPLC to afford Compound 436 (40 mg, 41%). EI-MS m/z: [M+H]⁺ 878.994.

### Example 80: Preparation of Compound 440

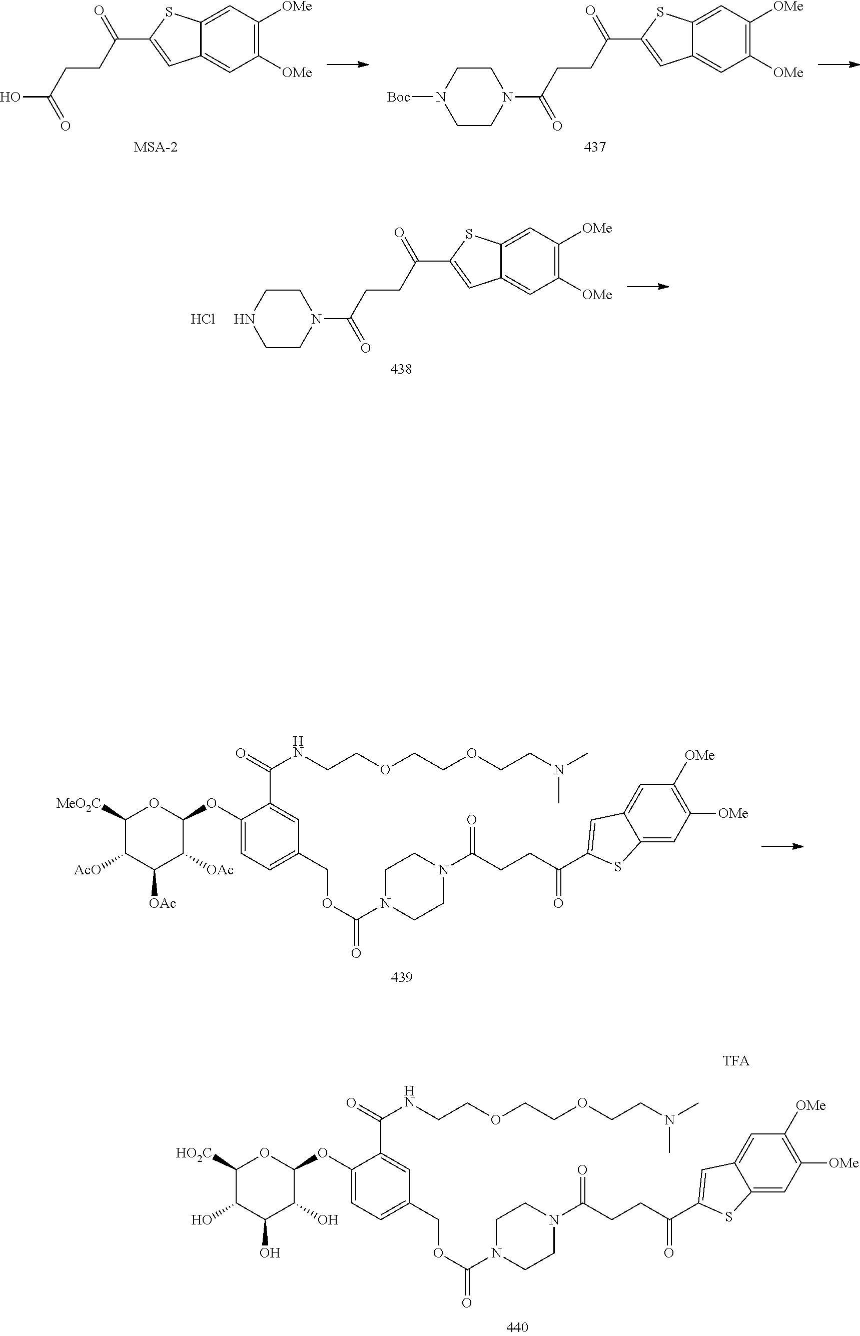

### Preparation of Intermediate Compound 437

To a solution of MSA-2 (444 mg, 1.51 mmol) in N,N-dimethylformamide (10 mL) were added N,N,N′,N′-tetramethyl-O-(1H-benzotriazol-1-yl)uronium hexafluorophosphate (HBTU, 858 mg, 2.26 mmol), 1-(tert-butoxycarbonyl)piperazine (309 mg, 1.65 mmol) and N,N′-diisopropylethylamine (0.78 mL, 4.52 mmol) at 0° C. under nitrogen. After stirred at room temperature for 18 hours, the reaction solution was diluted with ethyl acetate (50 mL) and washed with saturated aqueous sodium bicarbonate solution (50 mL) and distilled water (50 mL). The organic layer was dried over anhydrous sodium sulfate and filtered. The filtrate was concentrated under reduced pressure. The resulting residue was purified by column chromatography to afford Intermediate Compound 437 (650 mg, 93%). EI-MS m/z: [M+H]⁺ 463.05.

### Preparation of Intermediate Compound 438

To a solution of Intermediate 437 (650 mg, 1.41 mmol) in dichloromethane (4 mL) was added hydrochloric acid (4.0 M in 1,4-dioxane solution, 1 mL) at 0° C. under nitrogen. After stirred at room temperature for 2.5 hours, the solid obtained by diluted with diethyl ether was filtered and dried to afford Intermediate Compound 438 (559 mg, 99%), which was used without further purification. EI-MS m/z: [M+H]⁺ 363.17.

### Preparation of Intermediate Compound 439

To a solution of Intermediate Compound 438 (156 mg, 0.39 mmol) and Intermediate Compound 310 (315 mg, 0.39 mmol) in N,N-dimethylformamide (3 mL) was added N,N′-diisopropylethylamine (0.2 mL, 1.17 mmol) at 0° C. under nitrogen. After stirred at room temperature for 12 hours, the reaction solution was concentrated under reduced pressure. The resulting residue was purified by column chromatography to afford Intermediate Compound 439 (350 mg, 86%). EI-MS m/z: [M+H]⁺ 1030.90.

### Preparation of Compound 440

To a solution of Intermediate Compound 439 (113 mg, 0.34 mmol) in tetrahydrofuran (1 mL) and methanol (1 mL) was added lithium hydroxide monohydrate (64.1 mg, 1.52 mmol) in distilled water (1 mL) at −45° C. After stirred for 4 hours at 0° C., the reaction mixture was adjusted to pH 4-5 with acetic acid. The reaction solution was concentrated under reduced pressure. The resulting residue was purified by HPLC to afford Compound 440 (163 mg, 54%). EI-MS m/z: [M+H]⁺ 890.92.

### Example 81: Preparation of Compound 446

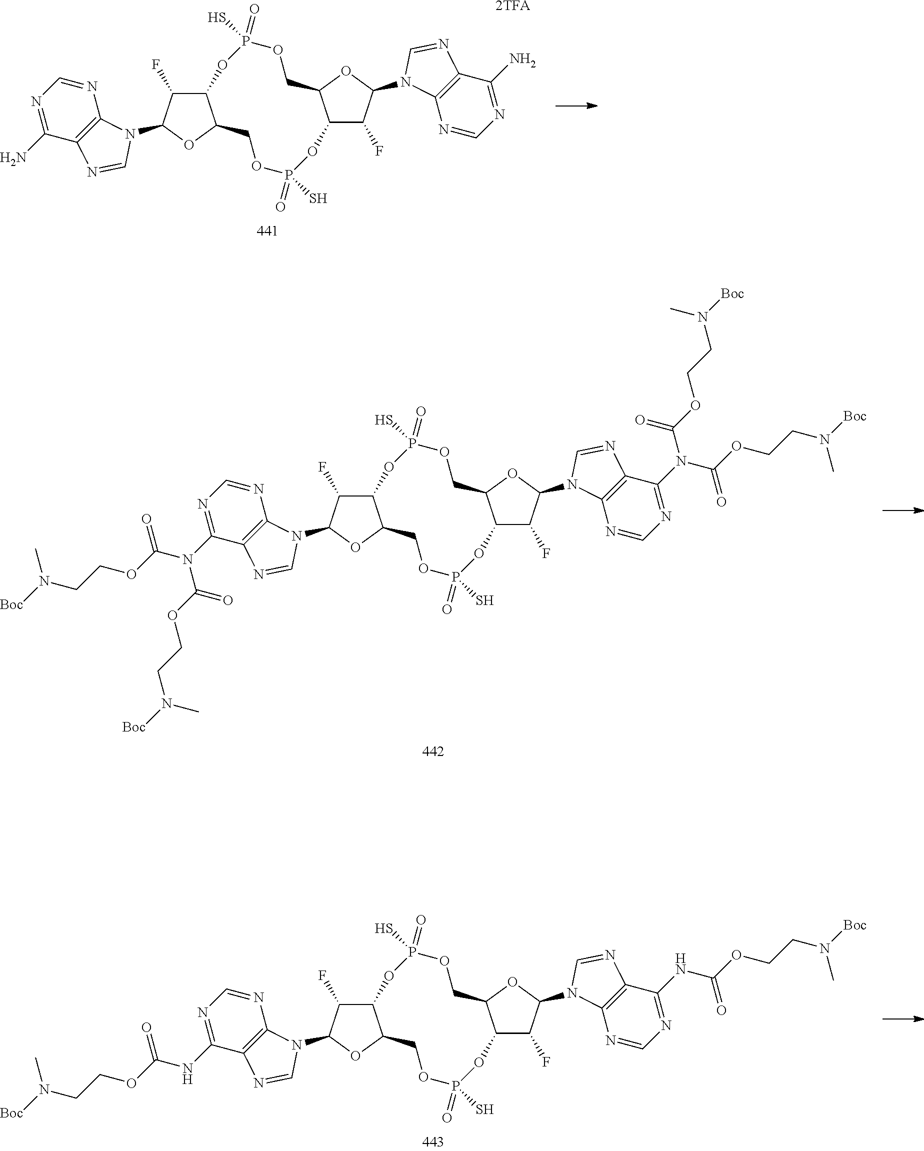

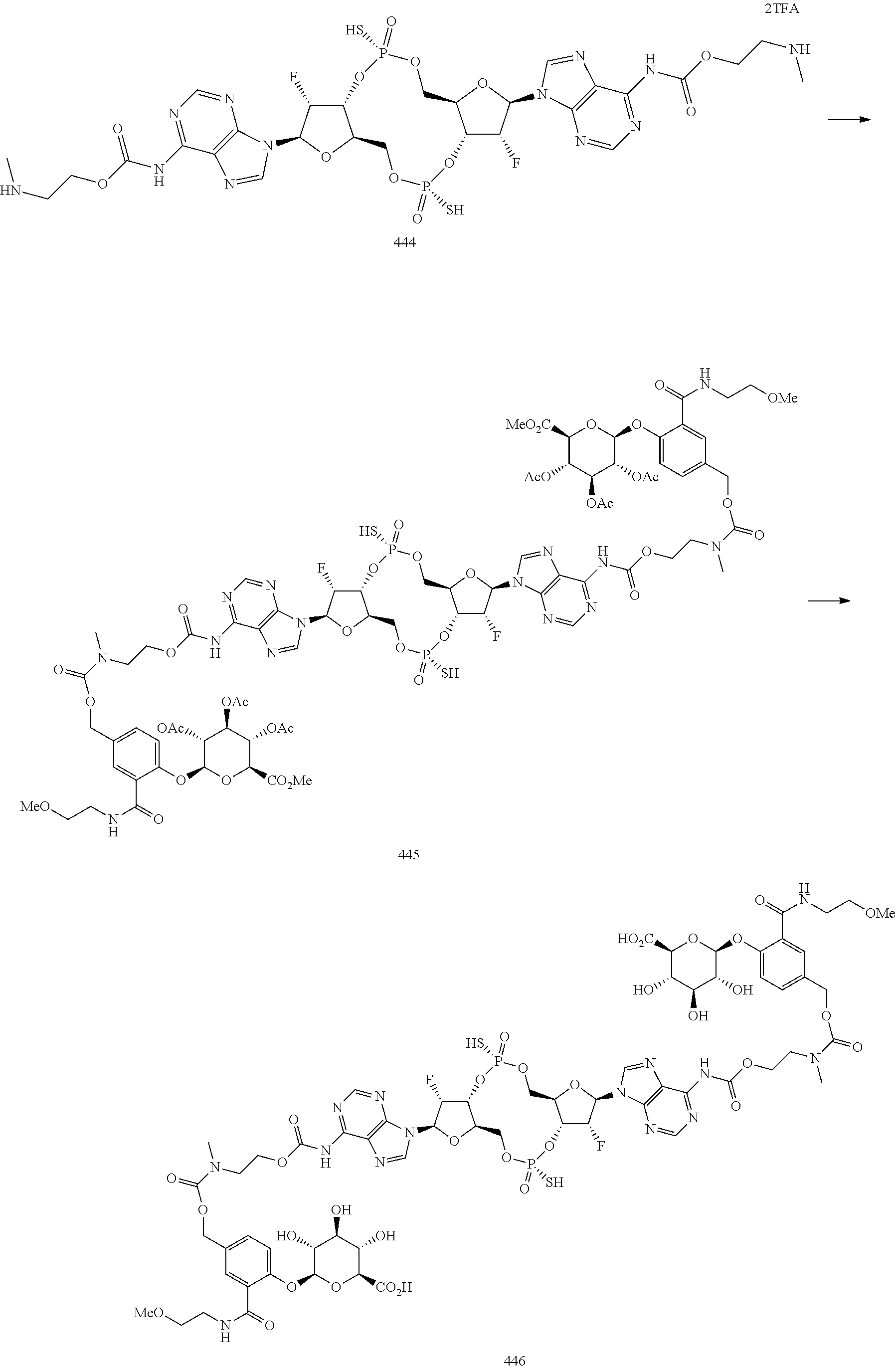

### Preparation of Intermediate Compound 442

To a solution of tert-butyl (2-hydroxyethyl)(methyl)carbamate (880 mg, 5.0 mmol) in dichloromethane (5 mL) were added pyridine (0.97 mL, 11.7 mmol) and phosgene solution (15 wt % in toluene, 7.2 mL, 10.8 mmol) in dichloromethane (15 mL) at −78° C. under nitrogen. After stirred at room temperature for 0.5 hours, the reaction solution Intermediate Compound 441 (50 mg, 0.054 mmol, Intermediate Compound 441 was prepared by the method described in the International patent publication No. WO 2020/092617 A1) in pyridine (2.5 mL) was added at 0° C. After stirred at room temperature for 4 hours, the reaction solution was diluted with dichloromethane (5 mL) and washed with and distilled water (15 mL). The organic layer was dried over anhydrous sodium sulfate and filtered. The filtrate was concentrated under reduced pressure. The resulting residue to afford Intermediate Compound 442 (120 mg), which was used without further purification. EI-MS m/z: [M+H]⁺ 1498.75.

### Preparation of Intermediate Compound 443

To a solution of Intermediate Compound 442 (120 mg) in methanol (2 mL) was added methylamine (33% in ethanol, 2 mL) at 0° C. under nitrogen. After stirred at room temperature for 2 hours, the reaction mixture was concentrated under reduced pressure. The resulting solid obtained by diluted with diethyl ether was filtered and dried to afford Intermediate Compound 443 (48 mg, crude), which was used without further purification. EI-MS m/z: [M+H]⁺ 1097.25.

### Preparation of Intermediate Compound 444

To a solution of Intermediate 443 (48 mg, crude) in dichloromethane (1 mL) was added trifluoroacetic acid (0.25 mL) at 0° C. under nitrogen. After stirred at room temperature for 2 hours, the reaction solution was concentrated under reduced pressure. The resulting solid obtained by diluted with methanol and acetonitrile was filtered and dried to afford Intermediate Compound 444 (59 mg, crude), which was used without further purification. EI-MS m/z: [M+H]⁺ 896.94.

### Preparation of Intermediate Compound 445

To a solution of Intermediate Compound 204 (78 mg, 0.104 mmol) and Intermediate Compound 444 (49 mg, 0.043 mmol) in N,N-dimethylformamide (2 mL) were added 1-hydroxy-7-azabenzotriazole (HOAt, 0.4 mg, 0.003 mmol) and N,N′-diisopropylethylamine (0.04 mL, 0.217 mmol) at 0° C. under nitrogen. After stirred at room temperature for 3 hours, the reaction solution was concentrated under reduced pressure to afford Intermediate Compound 445 (50 mg, crude), which was used without further purification. EI-MS m/z: [M+H]⁺ 2031.53.

### Preparation of Compound 446

To a solution of Intermediate Compound 445 (50 mg, crude) tetrahydrofuran (1 mL) and methanol (1 mL) was added tetramethylammonium hydroxide solution (10 wt % in water, 0.03 mL) and lithium hydroxide monohydrate (8.2 mg, 0.19 mmol) in distilled water (1 mL) at −45° C. After stirred at 0° C. for 2 hours, the reaction mixture was adjusted to pH 4-5 with acetic acid. The reaction solution was concentrated under reduced pressure. The resulting residue was purified by HPLC to afford Compound 446 (4.8 mg, 11%). EI-MS m/z: [M+H]⁺ 1751.32.

### Example 82: Preparation of Compound 447

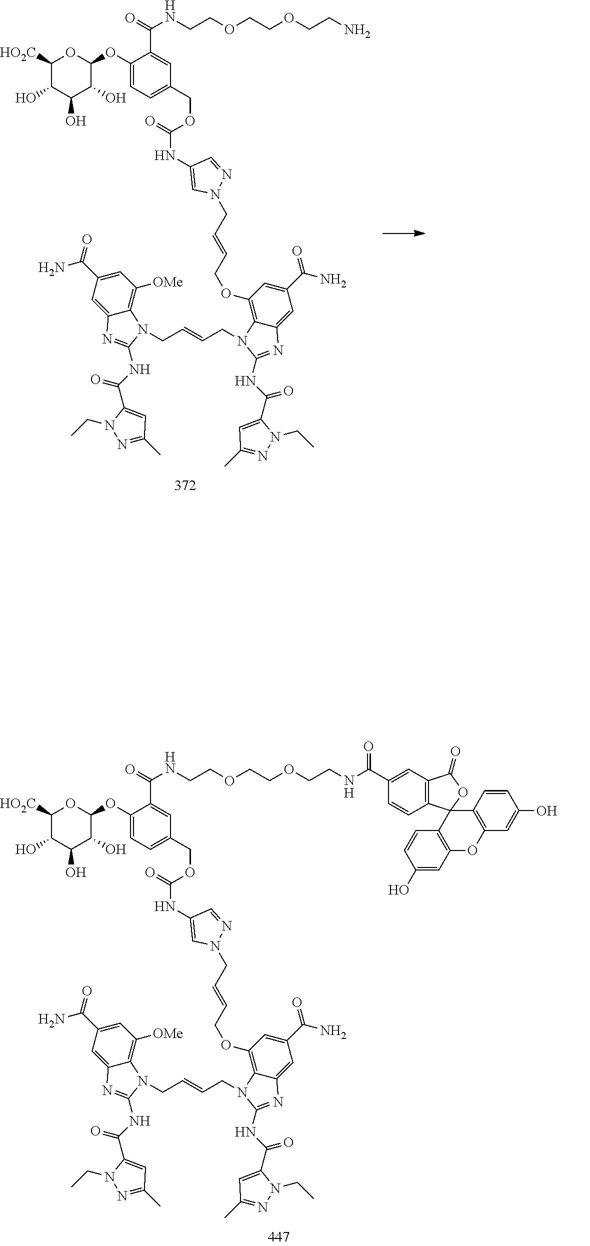 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCN)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCNC(=O)c5ccc6c(c5)C(=O)OC65c6ccc(O)cc6Oc6cc(O)ccc65)c4)cn3)c21]

### Preparation of Compound 447

To a solution of Compound 372 (70 mg, 0.04 mmol) and 5-carboxyfluorescein N-succinimidyl ester (21 mg, 0.044 mmol) in N,N-dimethylformamide (2 mL) was added triethylamine (0.03 mL, 0.21 mmol) at 0° C. under nitrogen. After stirred at room temperature for 3 hours, the reaction solution was concentrated under reduced pressure. The resulting residue was purified by HPLC to afford Compound 447 (47 mg).

¹H-NMR (400 MHz, DMSO) δ 10.15 (s, 2H), 9.50 (s, 1H), 8.89 (s, 1H), 8.46 (s, 1H), 8.30 (s, 1H), 8.27-8.22 (m, 1H), 7.94 (d, J=15.4 Hz, 2H), 7.83 (d, J=2.3 Hz, 1H), 7.67-7.60 (m, 2H), 7.47 (d, J=10.8 Hz, 1H), 7.40-7.28 (m, 5H), 7.24 (d, J=8.7 Hz, 1H), 6.68 (d, J=2.4 Hz, 1H), 6.58 (s, 1H), 6.55 (d, J=2.3 Hz, 1H), 6.50 (s, 1H), 5.78 (s, 2H), 5.14 (d, J=7.0 Hz, 2H), 5.05 (s, 2H), 4.89 (d, J=10.4 Hz, 3H), 4.52 (d, J=15.8 Hz, 6H), 3.97 (d, J=9.5 Hz, 1H), 3.69 (s, 3H), 3.56 (d, J=15.7 Hz, 7H), 2.11-2.03 (m, 5H), 1.91 (s, 1H), 1.24 (q, J=6.8 Hz, 5H).

### Example 83: Preparation of Compound 453

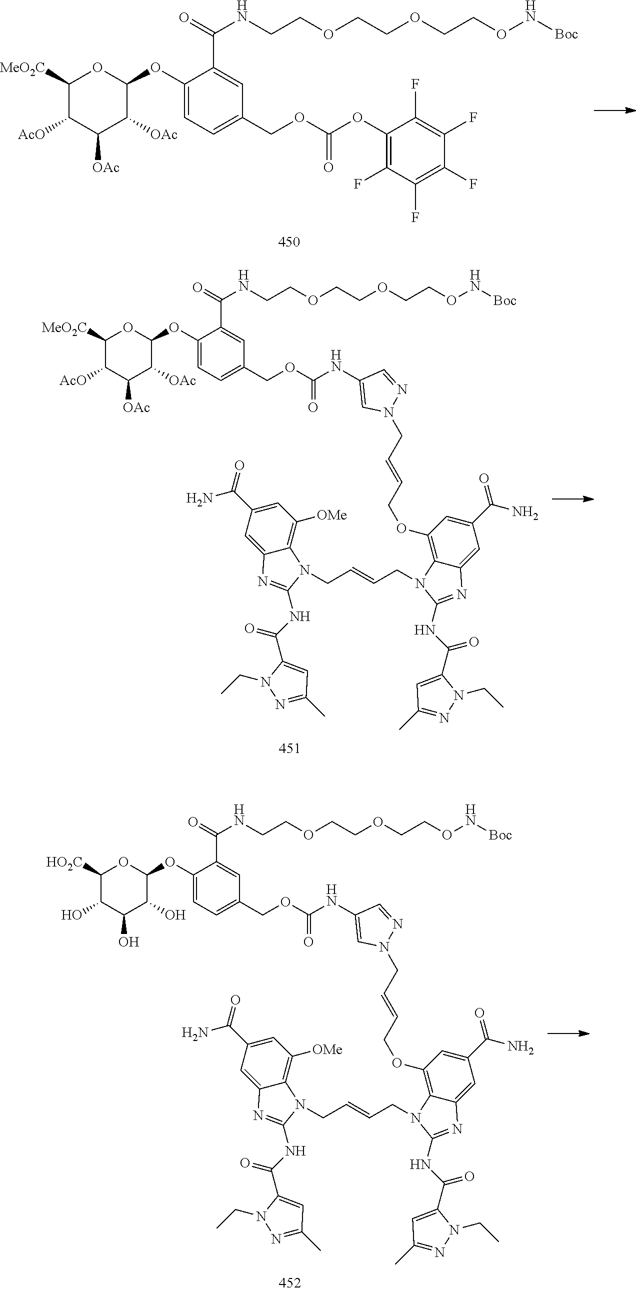

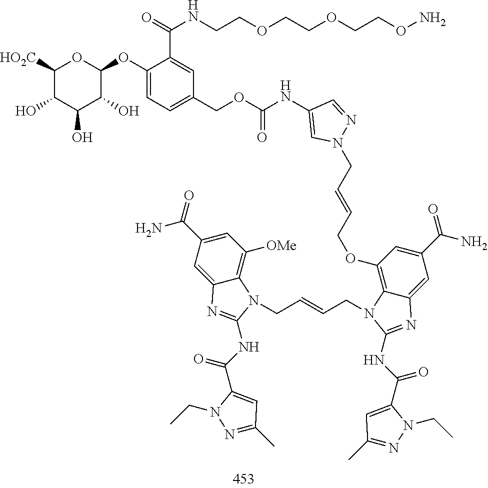 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)cn3)c21]

### Preparation of Intermediate Compound 451

To a solution of Intermediate Compound 450 (25.5 mg, 0.03 mmol, Intermediate Compound 450 was prepared by the method described in the Korean Patent Application No. 10-2023-0099038 and Intermediate Compound 55 (30 mg, 0.02 mmol) in N,N-dimethylformamide (1 mL) were added 1-hydroxy-7-azabenzotriazole (HOAt, 0.7 mg, 0.005 mmol) and N,N′-diisopropylethylamine (0.02 mL, 0.12 mmol) at 0° C. under nitrogen. After stirred at room temperature for 2 hours, the reaction solution was concentrated under reduced pressure to afford Intermediate Compound 451 (36 mg, crude), which was used without further purification. EI-MS m/z: [M/2+H]⁺ 759.25.

### Preparation of Intermediate Compound 452

To a solution of Intermediate Compound 451 (36 mg, crude) in methanol (0.5 mL) was added lithium hydroxide monohydrate (5.1 mg, 0.12 mmol) in distilled water (0.5 mL) at −50° C. After stirred for 2 hours at 0° C., the reaction mixture was adjusted to pH 4-5 with acetic acid. The reaction solution was concentrated under reduced pressure to afford Intermediate Compound 452 (36 mg, crude), which was used without further purification. EI-MS m/z: [M/2+H]⁺746.27.

### Preparation of Compound 453

To a solution of Intermediate 452 (36 mg) in dichloromethane (1 mL) was added trifluoroacetic acid (0.35 mL) at 0° C. under nitrogen. After stirred at room temperature for 2 hours under nitrogen, the reaction solution was concentrated under reduced pressure. The resulting residue was purified by HPLC to afford Compound 453 (25 mg, 63%). EI-MS m/z: [M+H]⁺ 1376.63, [M/2+H]⁺689.09.

### Example 84: Preparation of Compounds 454 to 500

Compounds 454 through 500, as shown in Table A below, were prepared according to procedures analogous to those outlined in Example 83 above using the appropriate monomers described as Examples above, or as obtained from commercial sources, in the coupling and deprotected step.

TABLE A

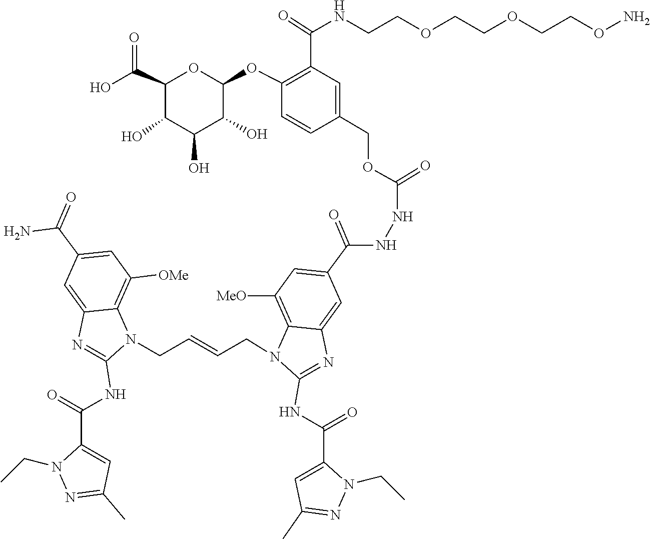 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NNC(=O)OCc3ccc(O[C@@H]4O[C@H](C(=O)O)[C@@H](O)[C@H](O)[C@H]4O)c(C(=O)NCCOCCOCCON)c3)cc(OC)c21]

Compound 454

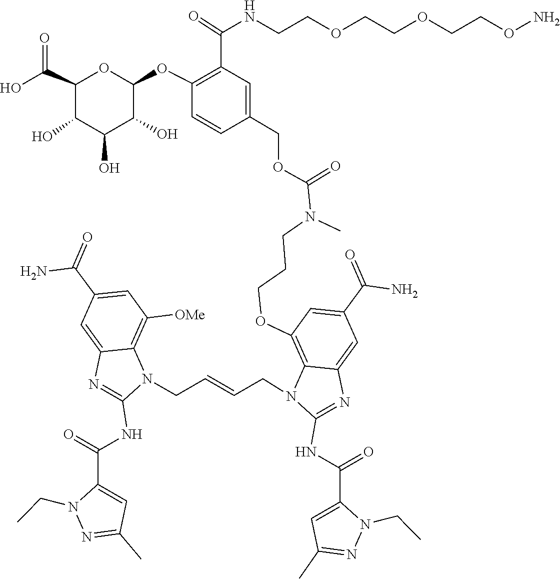 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

Compound 455

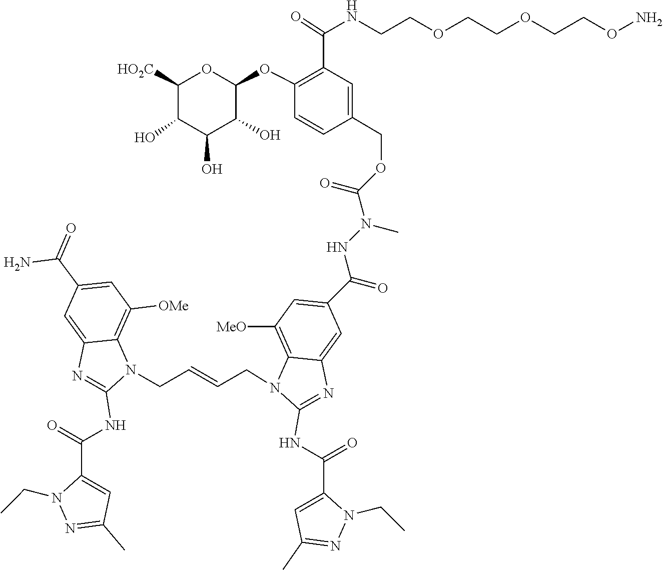 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN(C)C(=O)OCc3ccc(O[C@@H]4O[C@H](C(=O)O)[C@@H](O)[C@H](O)[C@H]4O)c(C(=O)NCCOCCOCCON)c3)cc(OC)c21]

Compound 456

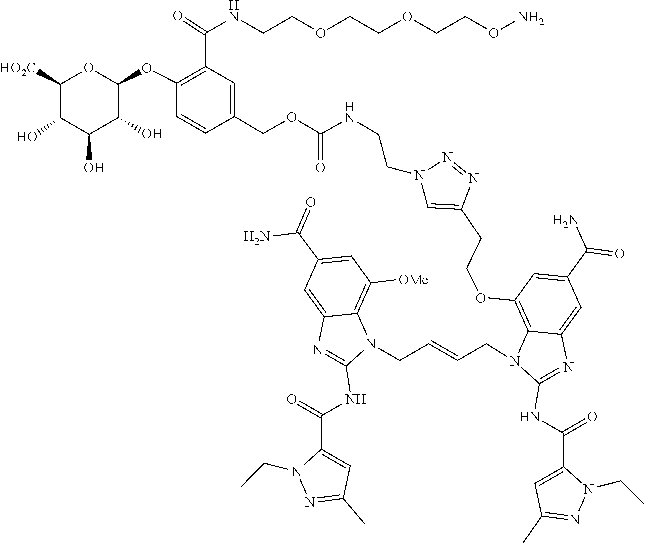 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3cn(CCNC(=O)OCc4ccc(O[C@@H]5O[C@H](C(=O)O)[C@@H](O)[C@H](O)[C@H]5O)c(C(=O)NCCOCCOCCON)c4)nn3)c21]

Compound 457

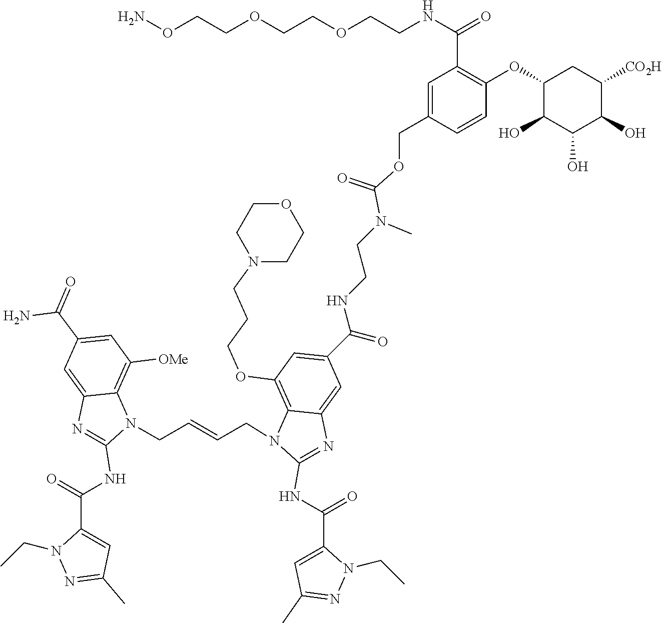 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NCCN(C)C(=O)OCc3ccc(O[C@@H]4C[C@H](C(=O)O)[C@@H](O)[C@H](O)[C@H]4O)c(C(=O)NCCOCCOCCON)c3)cc(OCCCN3CCOCC3)c21]

Compound 458

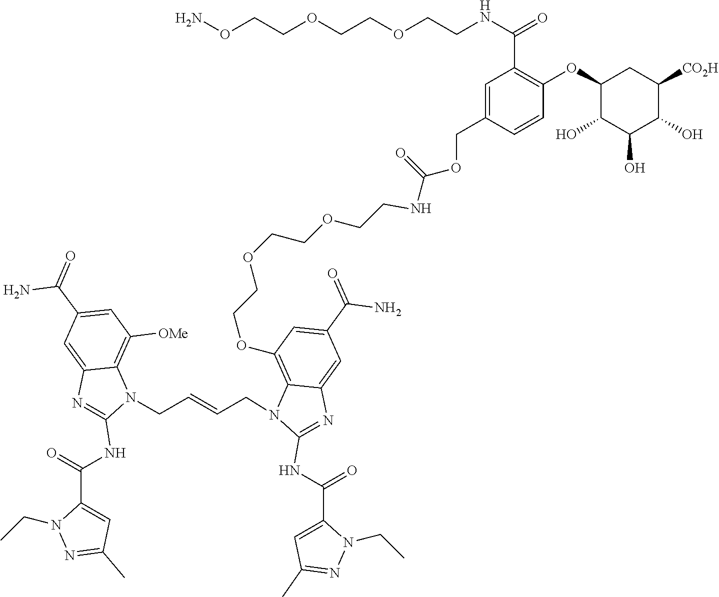 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCOCCOCCNC(=O)OCc3ccc(O[C@H]4C[C@@H](C(=O)O)[C@H](O)[C@@H](O)[C@@H]4O)c(C(=O)NCCOCCOCCON)c3)c21]

Compound 459

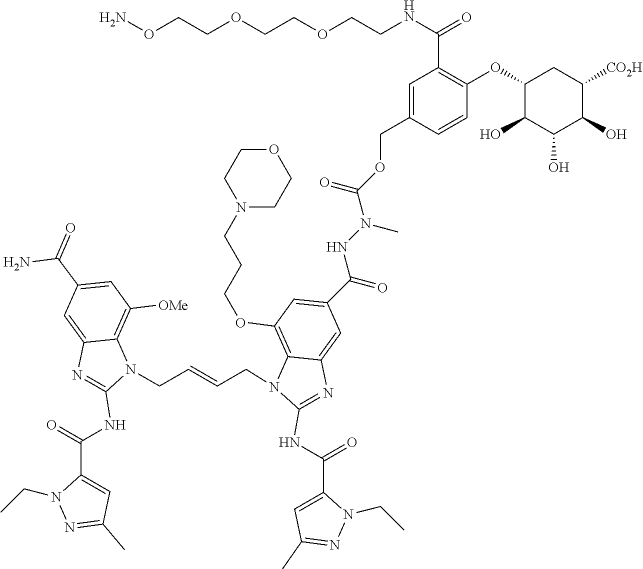 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN(C)C(=O)OCc3ccc(O[C@@H]4C[C@H](C(=O)O)[C@@H](O)[C@H](O)[C@H]4O)c(C(=O)NCCOCCOCCON)c3)cc(OCCCN3CCOCC3)c21]

Compound 460

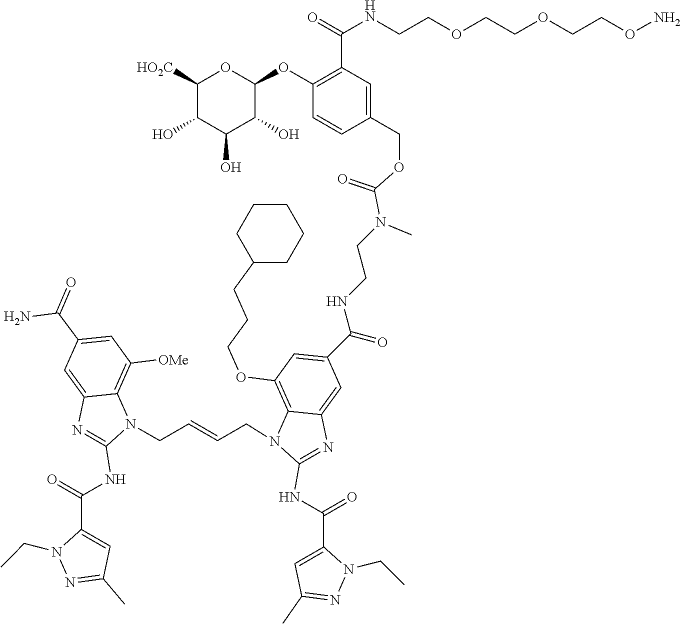 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NCCN(C)C(=O)OCc3ccc(O[C@@H]4O[C@H](C(=O)O)[C@@H](O)[C@H](O)[C@H]4O)c(C(=O)NCCOCCOCCON)c3)cc(OCCCC3CCCCC3)c21]

Compound 461

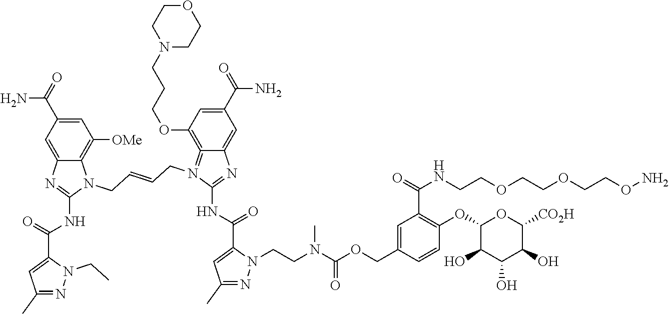

Compound 462

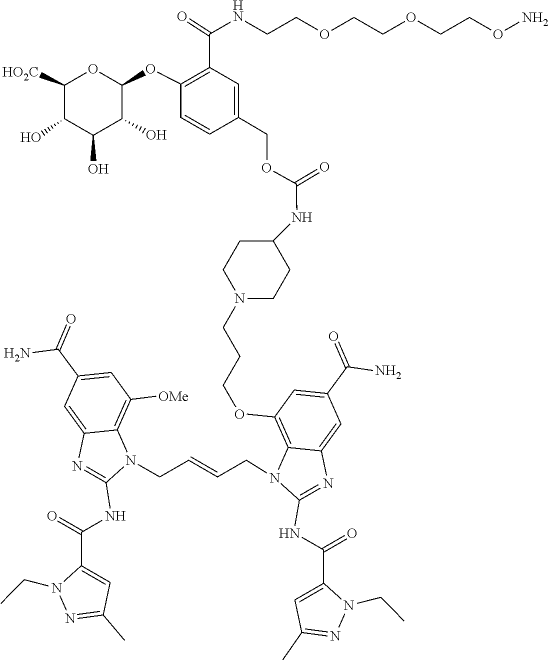 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c21]

Compound 463

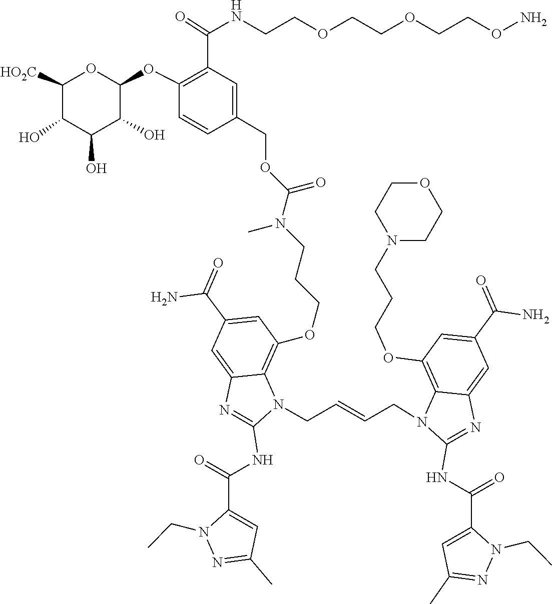 [SMILES: CCn1nc(C)cc1C(=O)Nc1nc2cc(C(N)=O)cc(OCCCN3CCOCC3)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

Compound 464

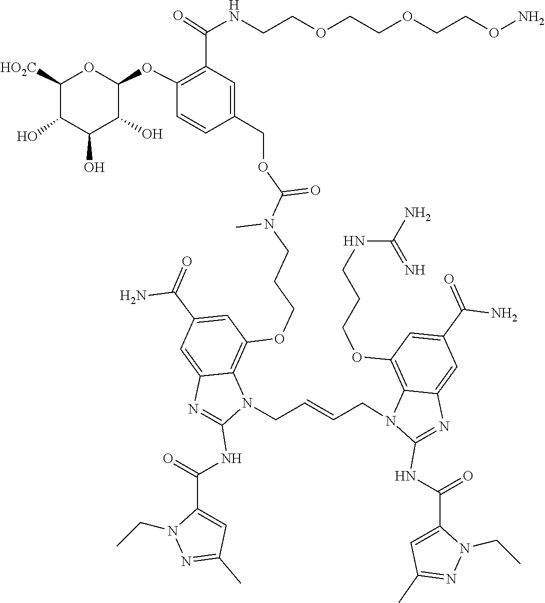 [SMILES: CCn1nc(C)cc1C(=O)Nc1nc2cc(C(N)=O)cc(OCCCNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

Compound 465

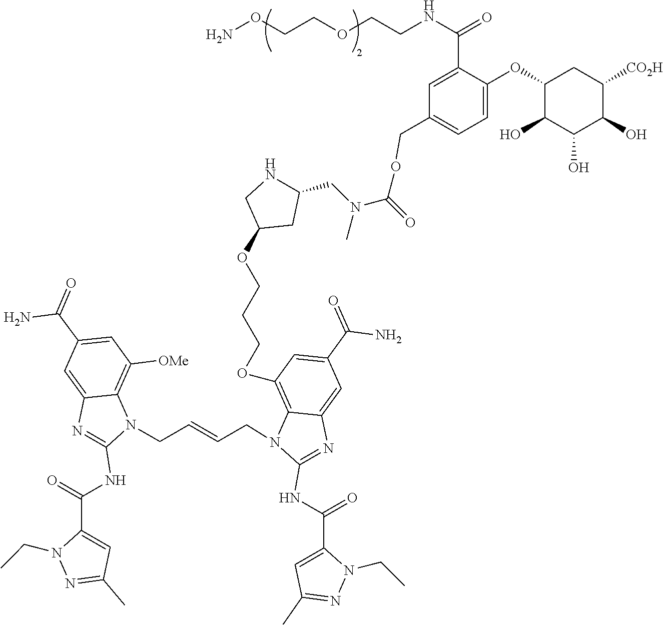

Compound 466

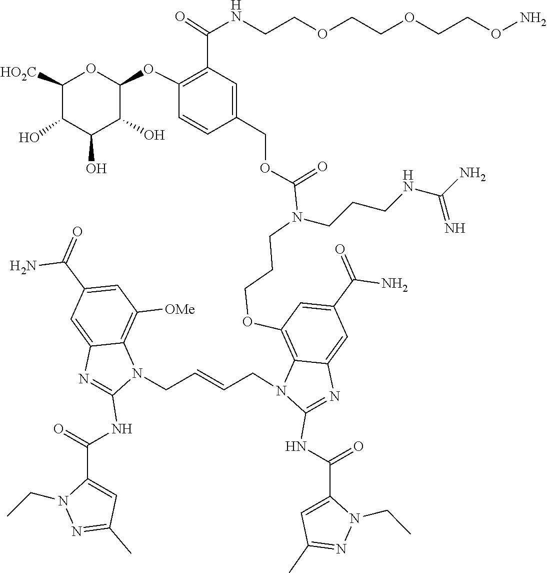 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(CCCNC(=N)N)C(=O)OCc3ccc(O[C@@H]4O[C@H](C(=O)O)[C@@H](O)[C@H](O)[C@H]4O)c(C(=O)NCCOCCOCCON)c3)c21]

Compound 467

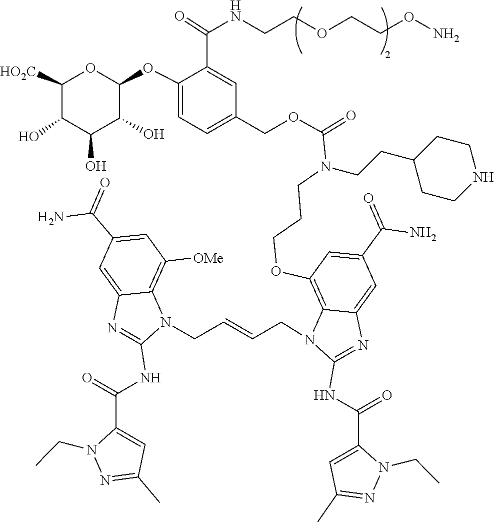

Compound 468

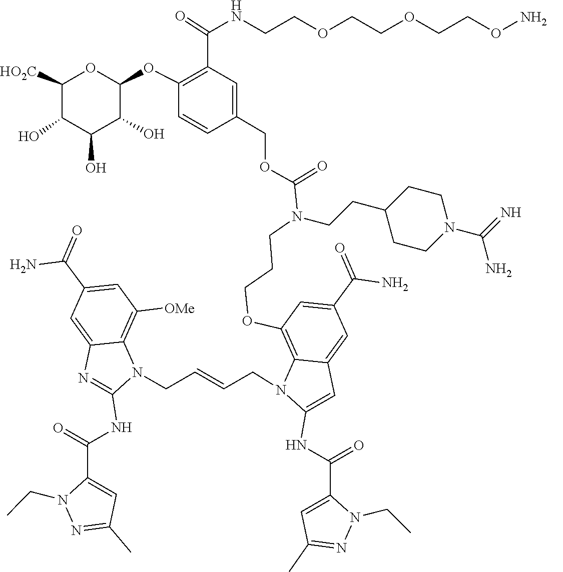 [SMILES: CCn1nc(C)cc1C(=O)Nc1cc2cc(C(N)=O)cc(OCCCN(CCC3CCN(C(=N)N)CC3)C(=O)OCc3ccc(O[C@@H]4O[C@H](C(=O)O)[C@@H](O)[C@H](O)[C@H]4O)c(C(=O)NCCOCCOCCON)c3)c2n1C/C=C/Cn1c(NC(=O)c2cc(C)nn2CC)nc2cc(C(N)=O)cc(OC)c21]

Compound 469

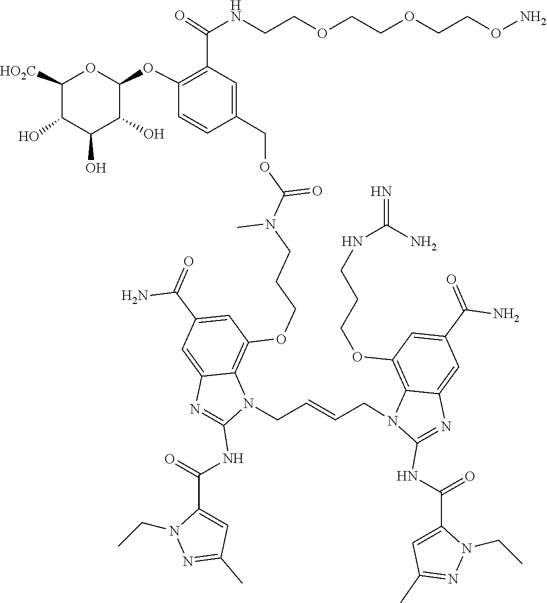 [SMILES: CCn1nc(C)cc1C(=O)Nc1nc2cc(C(N)=O)cc(OCCCNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

Compound 470

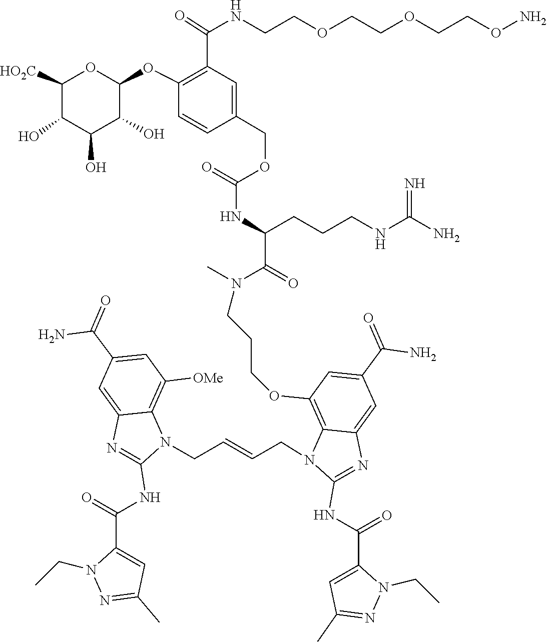 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CCCNC(=N)N)NC(=O)OCc3ccc(O[C@@H]4O[C@H](C(=O)O)[C@@H](O)[C@H](O)[C@H]4O)c(C(=O)NCCOCCOCCON)c3)c21]

Compound 471

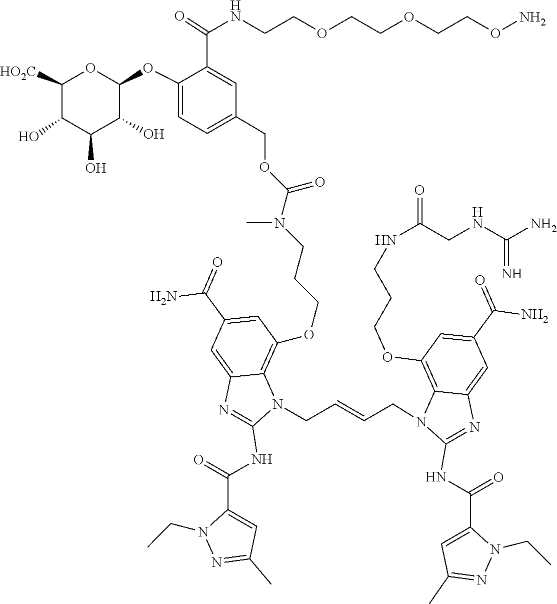 [SMILES: CCn1nc(C)cc1C(=O)Nc1nc2cc(C(N)=O)cc(OCCCNC(=O)CNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

Compound 472

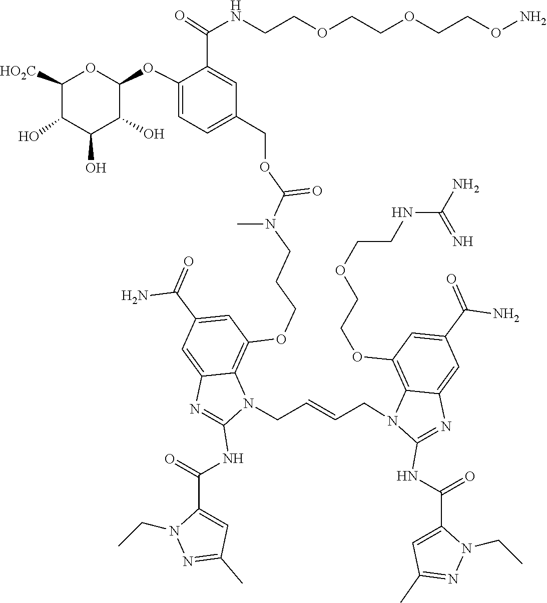 [SMILES: CCn1nc(C)cc1C(=O)Nc1nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c2n1C/C=C/Cn1c(NC(=O)c2cc(C)nn2CC)nc2cc(C(N)=O)cc(OCCOCCNC(=N)N)c21]

Compound 473

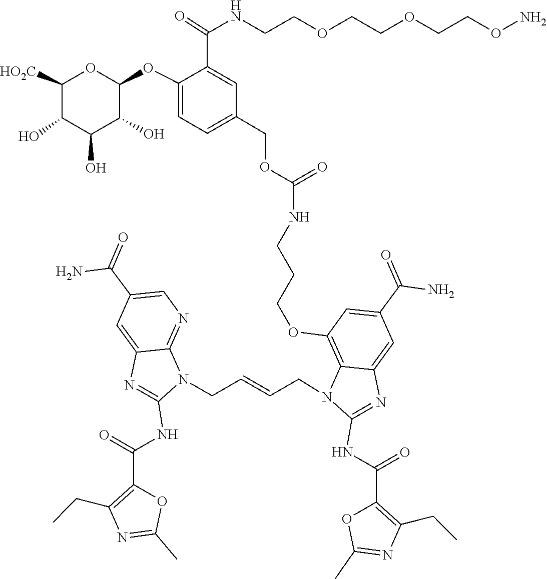 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNC(=O)OCc3ccc(O[C@@H]4O[C@H](C(=O)O)[C@@H](O)[C@H](O)[C@H]4O)c(C(=O)NCCOCCOCCON)c3)c21]

Compound 474

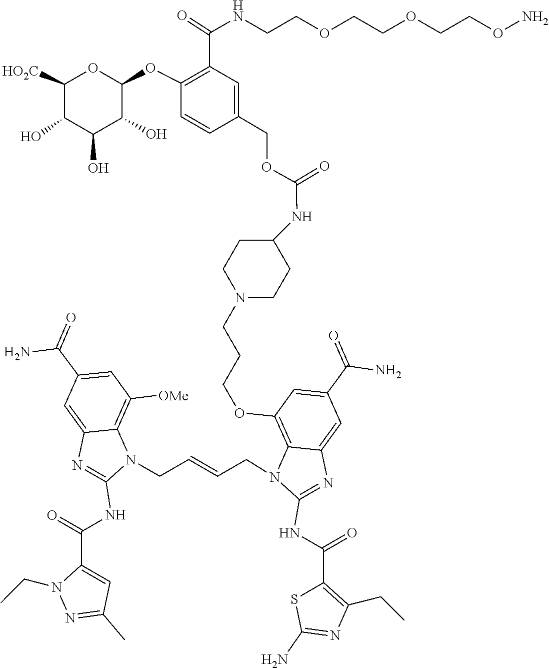 [SMILES: CCc1nc(N)sc1C(=O)Nc1nc2cc(C(N)=O)cc(OCCCN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c2n1C/C=C/Cn1c(NC(=O)c2cc(C)nn2CC)nc2cc(C(N)=O)cc(OC)c21]

Compound 475

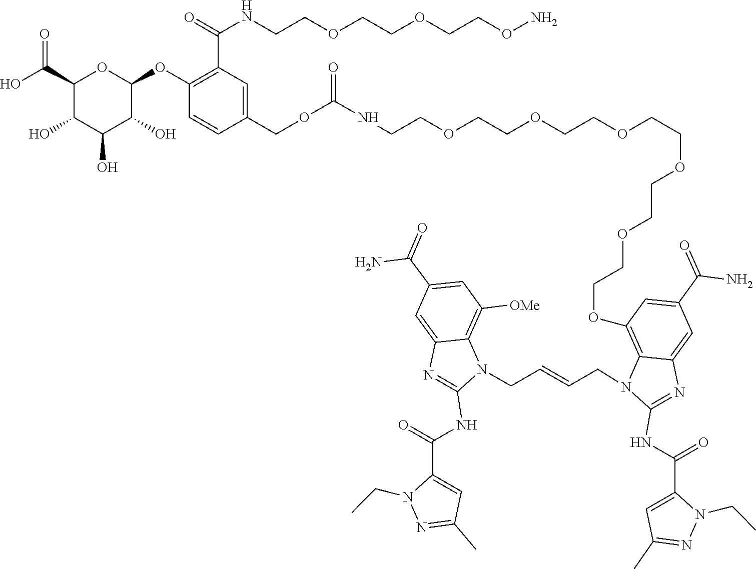 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCOCCOCCOCCOCCOCCNC(=O)OCc3ccc(O[C@@H]4O[C@H](C(=O)O)[C@@H](O)[C@H](O)[C@H]4O)c(C(=O)NCCOCCOCCON)c3)c21]

Compound 476

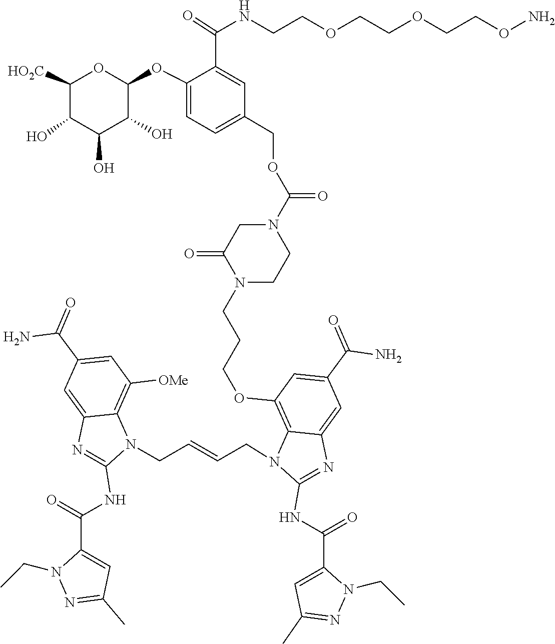 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(O[C@@H]5O[C@H](C(=O)O)[C@@H](O)[C@H](O)[C@H]5O)c(C(=O)NCCOCCOCCON)c4)CC3=O)c21]

Compound 477

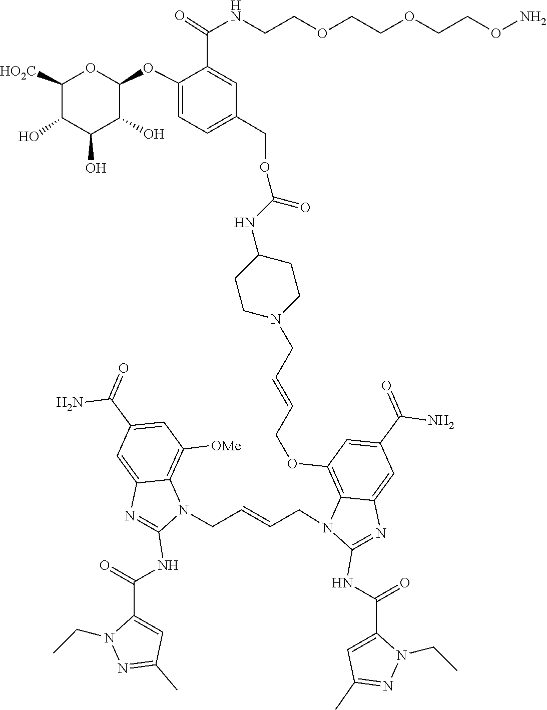 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c21]

Compound 478

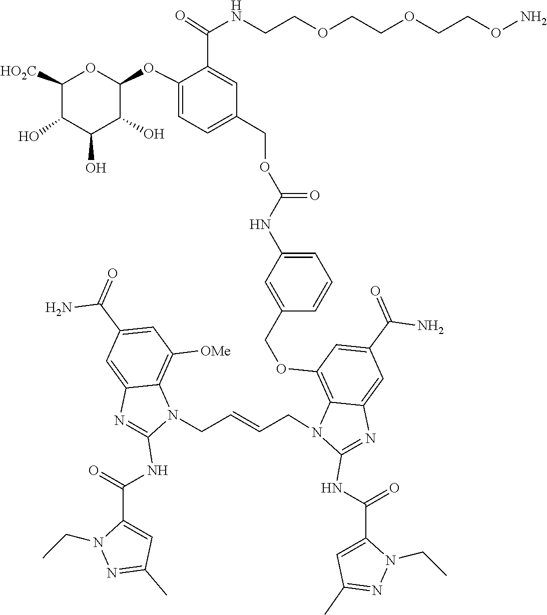 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCc3cccc(NC(=O)OCc4ccc(O[C@@H]5O[C@H](C(=O)O)[C@@H](O)[C@H](O)[C@H]5O)c(C(=O)NCCOCCOCCON)c4)c3)c21]

Compound 479

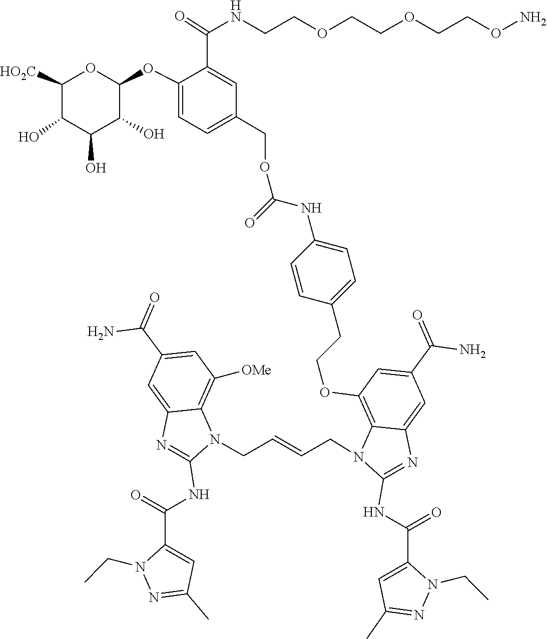 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOCCOCCON)c4)cc3)c21]

Compound 480

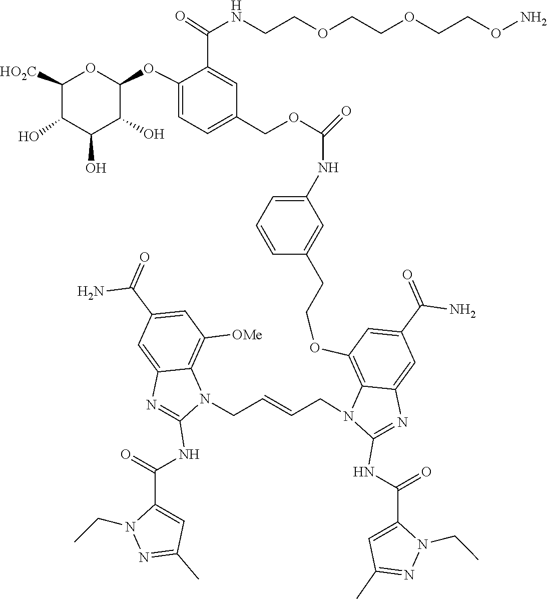 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3cccc(NC(=O)OCc4ccc(O[C@@H]5O[C@H](C(=O)O)[C@@H](O)[C@H](O)[C@H]5O)c(C(=O)NCCOCCOCCON)c4)c3)c21]

Compound 481

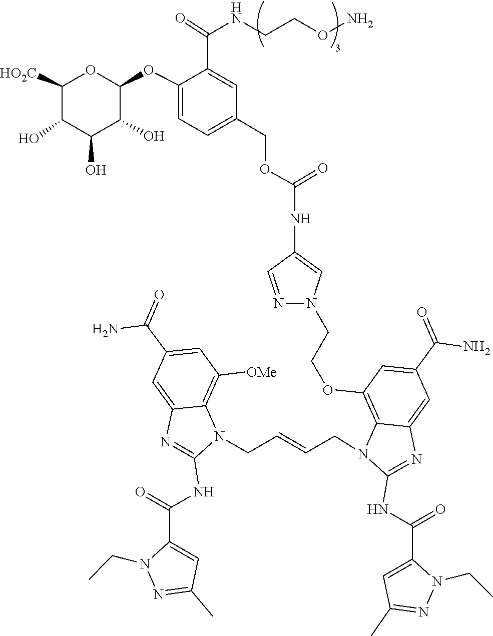

Compound 482

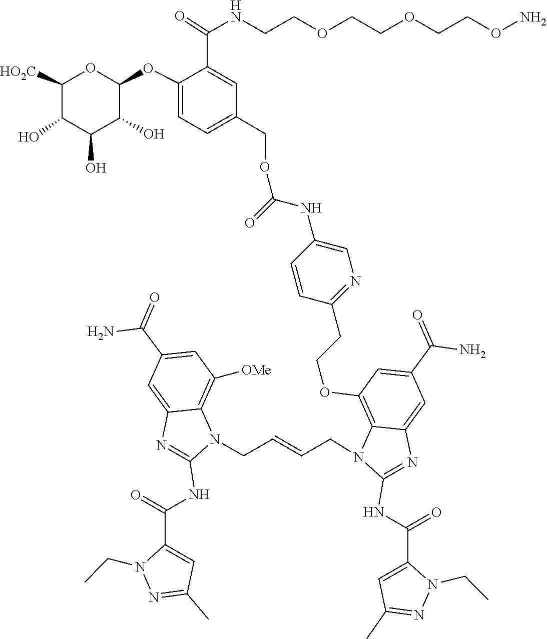 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOCCOCCON)c4)cn3)c21]

Compound 483

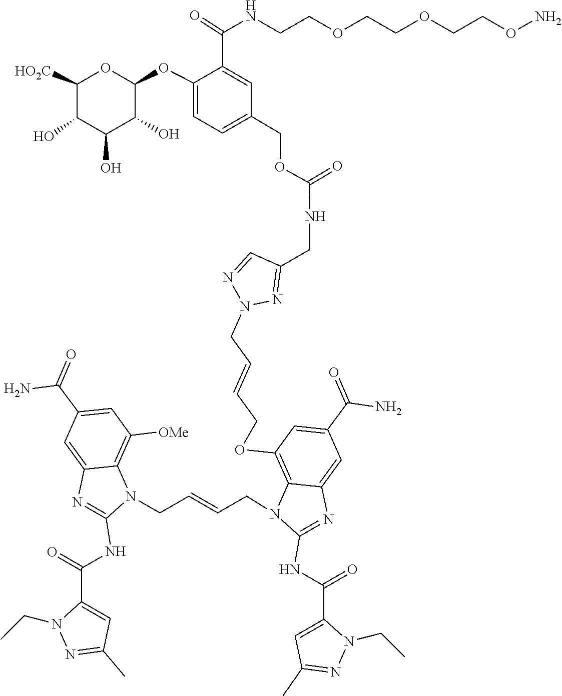 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3ncc(CNC(=O)OCc4ccc(O[C@@H]5O[C@H](C(=O)O)[C@@H](O)[C@H](O)[C@H]5O)c(C(=O)NCCOCCOCCON)c4)n3)c21]

Compound 484

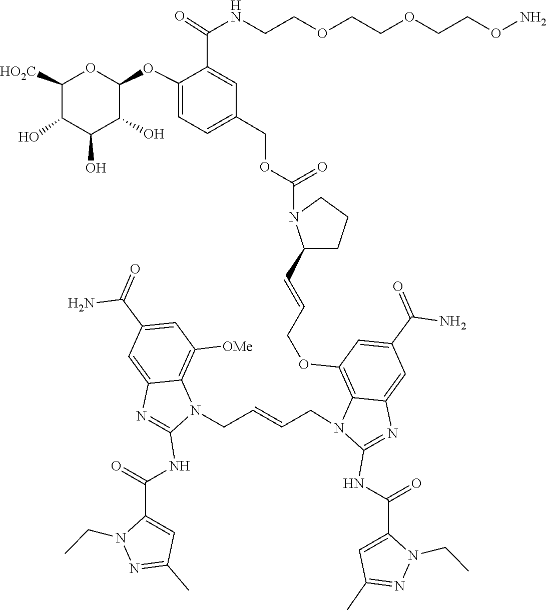 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/[C@@H]3CCCN3C(=O)OCc3ccc(O[C@@H]4O[C@H](C(=O)O)[C@@H](O)[C@H](O)[C@H]4O)c(C(=O)NCCOCCOCCON)c3)c21]

Compound 485

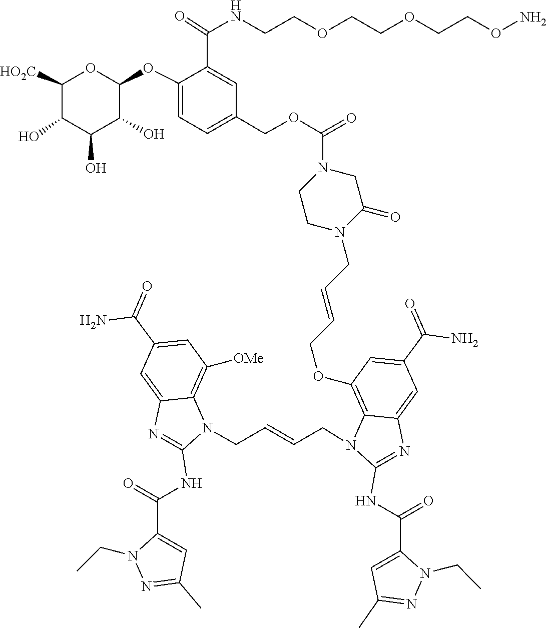 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)O)[C@@H](O)[C@H](O)[C@H]5O)c(C(=O)NCCOCCOCCON)c4)CC3=O)c21]

Compound 486

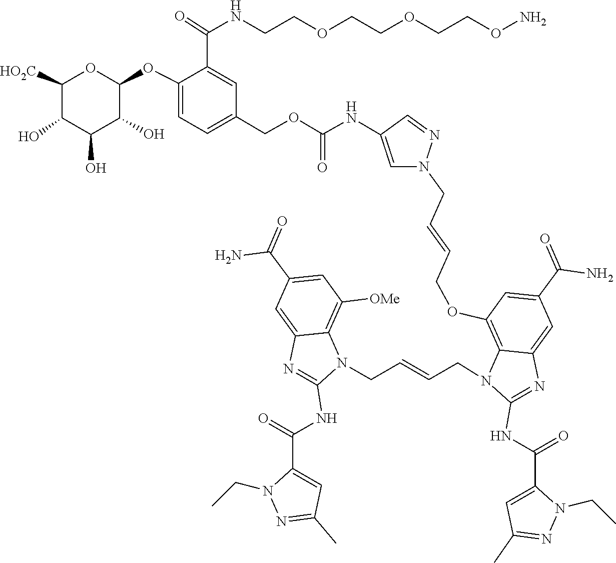 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)cn3)c21]

Compound 487

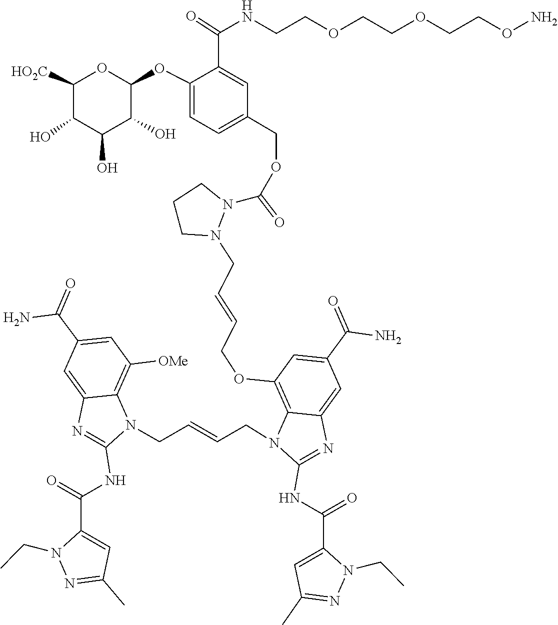 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOCCOCCON)c3)c21]

Compound 488

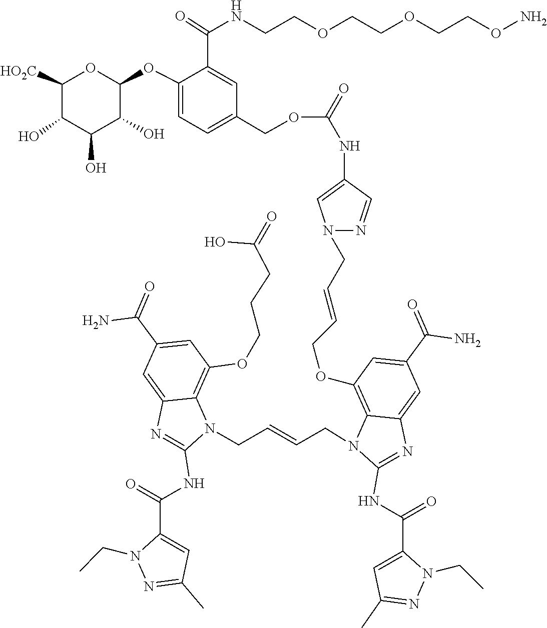 [SMILES: CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)cn3)c2n1C/C=C/Cn1c(NC(=O)c2cc(C)nn2CC)nc2cc(C(N)=O)cc(OCCCC(=O)O)c21]

Compound 489

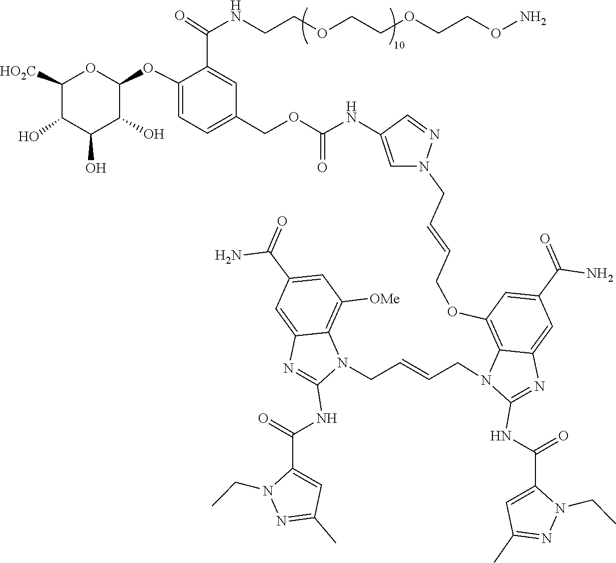

Compound 490

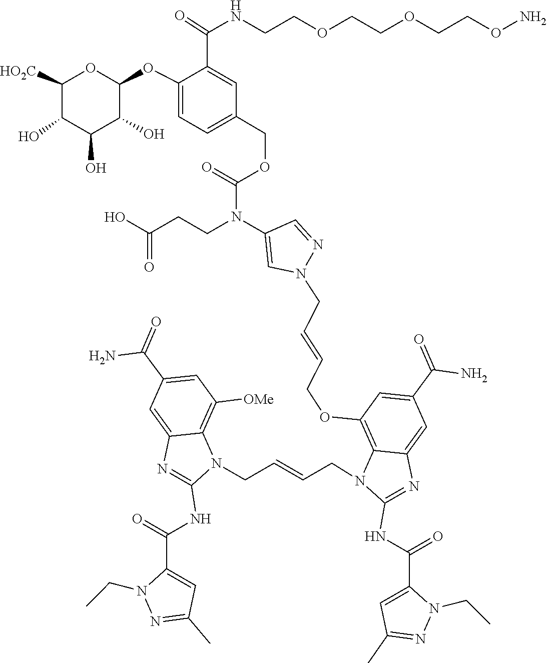 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(N(CCC(=O)O)C(=O)OCc4ccc(O[C@@H]5O[C@H](C(=O)O)[C@@H](O)[C@H](O)[C@H]5O)c(C(=O)NCCOCCOCCON)c4)cn3)c21]

Compound 491

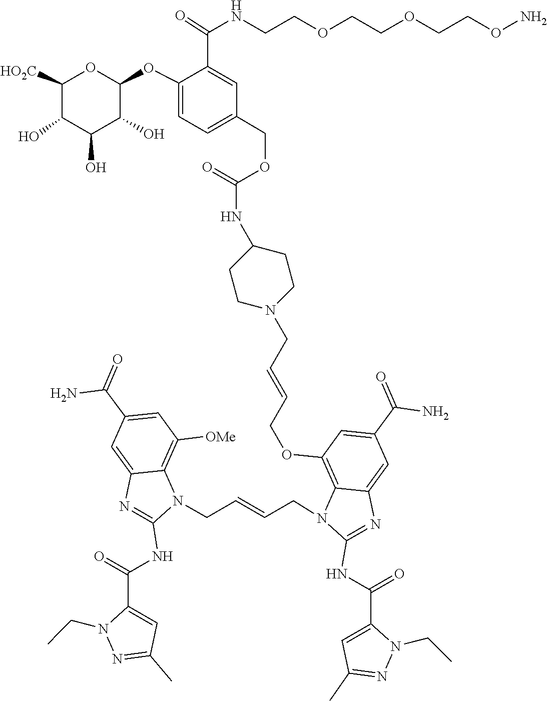 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c21]

Compound 492

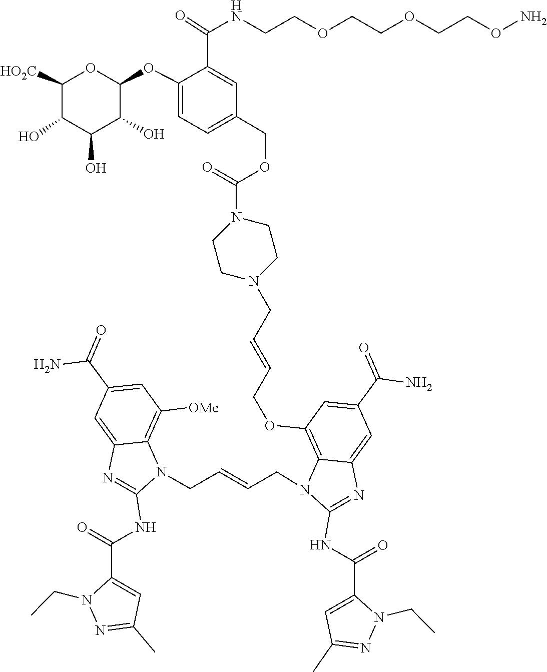 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)O)[C@@H](O)[C@H](O)[C@H]5O)c(C(=O)NCCOCCOCCON)c4)CC3)c21]

Compound 493

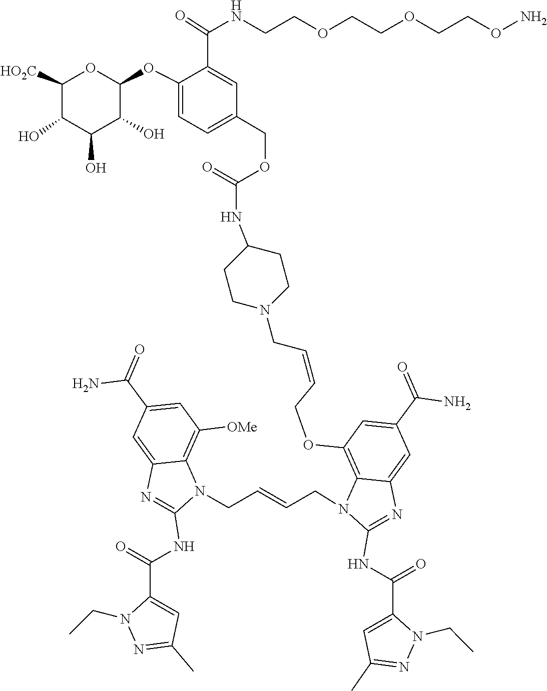 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C\CN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c21]

Compound 494

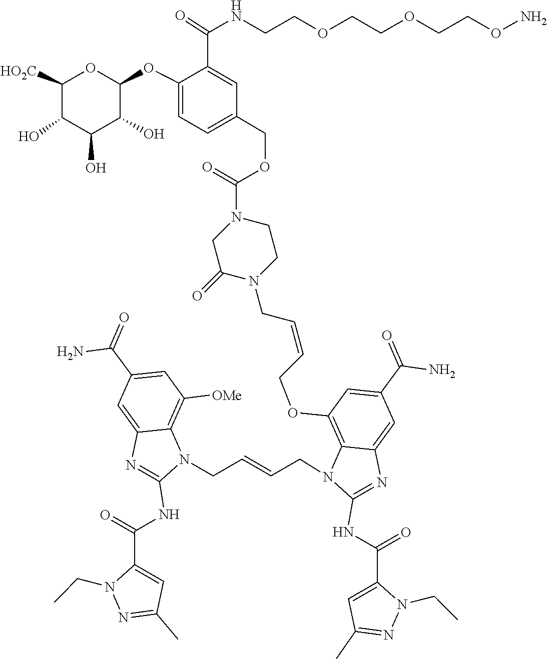 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C\CN3CCN(C(=O)OCc4ccc(O[C@@H]5O[C@H](C(=O)O)[C@@H](O)[C@H](O)[C@H]5O)c(C(=O)NCCOCCOCCON)c4)CC3=O)c21]

Compound 495

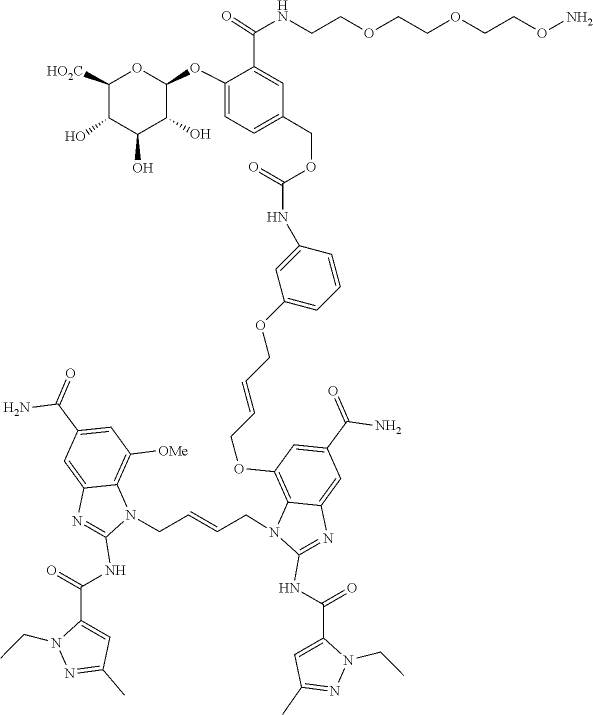 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/COc3cccc(NC(=O)OCc4ccc(O[C@@H]5O[C@H](C(=O)O)[C@@H](O)[C@H](O)[C@H]5O)c(C(=O)NCCOCCOCCON)c4)c3)c21]

Compound 496

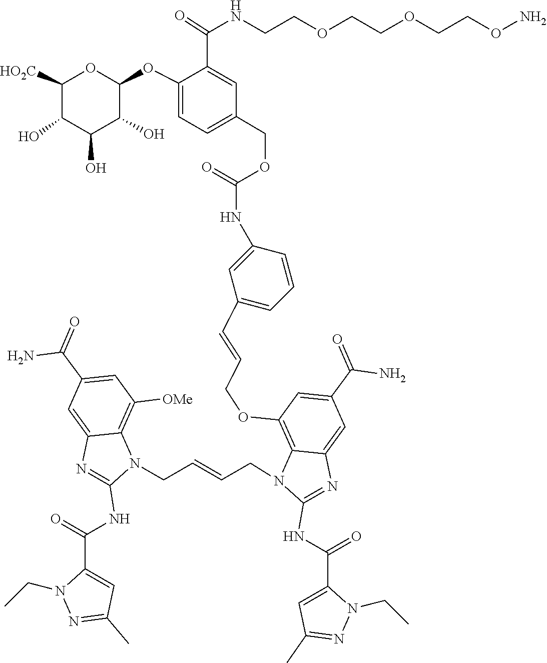 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/c3cccc(NC(=O)OCc4ccc(O[C@@H]5O[C@H](C(=O)O)[C@@H](O)[C@H](O)[C@H]5O)c(C(=O)NCCOCCOCCON)c4)c3)c21]

Compound 497

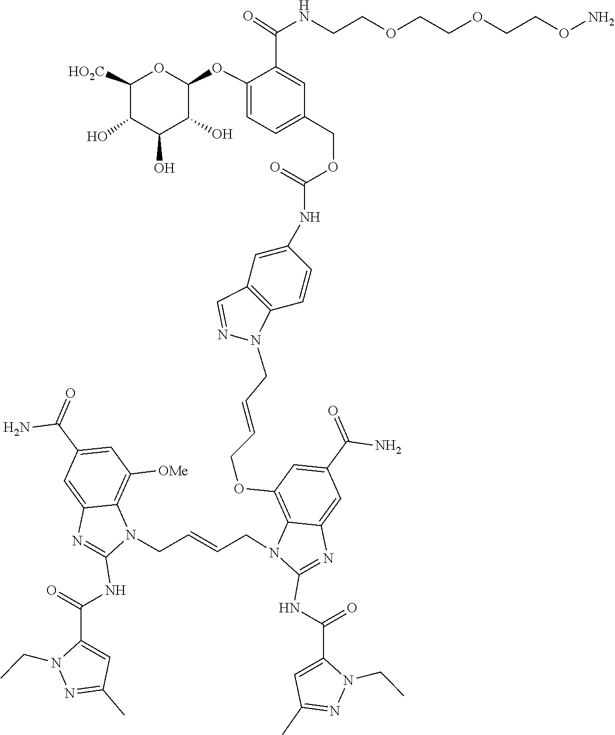 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3ncc4cc(NC(=O)OCc5ccc(O[C@@H]6O[C@H](C(=O)O)[C@@H](O)[C@H](O)[C@H]6O)c(C(=O)NCCOCCOCCON)c5)ccc43)c21]

Compound 498

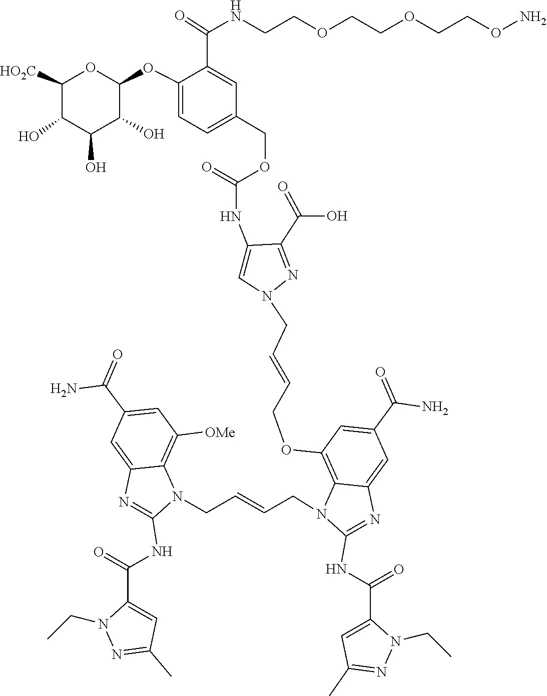 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)c(C(=O)O)n3)c21]

Compound 499

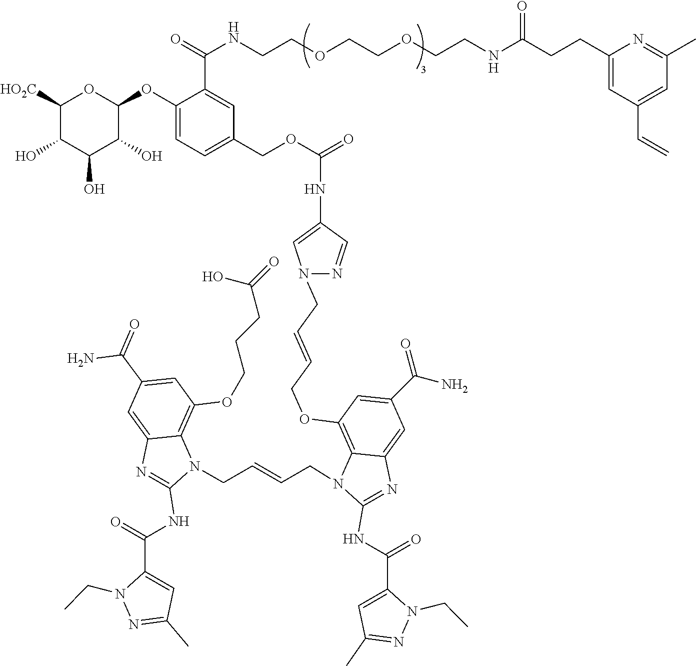

Compound 500

### Example 86: Preparation of Comparative Compound #1

The Compound of Comparative Compound 1 has the following structure and was purchased from ChemScene (#CS-0077291) and used.

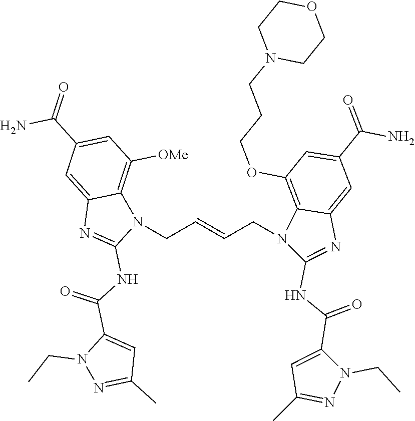 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOCC3)c21]

### Example 87: Preparation of Comparative Compound #2

The compound of Comparative Compound #2 has the following structure and was prepared by the method described in the United States patent publication No. 2021-0032269 A1.

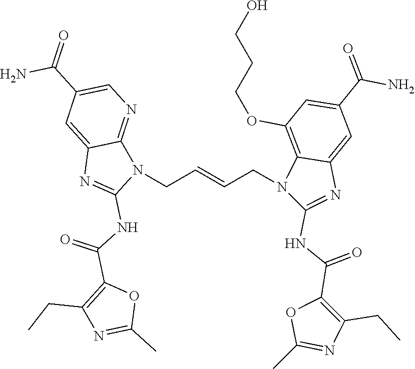 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCO)c21]

### Example 88: Preparation of Comparative Compound #3

The compound of Comparative Compound #3 has the following structure and was prepared by the method described in the United States patent publication No. 2022-0267364 A1.

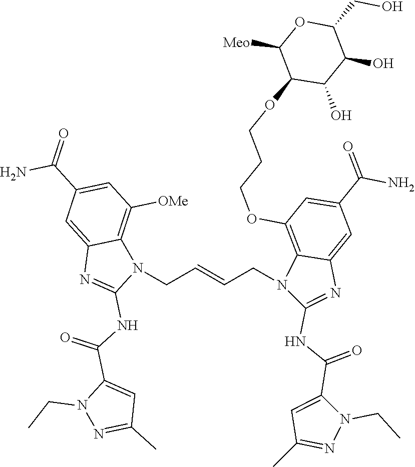

### Testing of Compounds

### Experimental Example 1: Assessment of STING Activation and Direct STING Binding

To assess potency of STING agonist compounds described herein, THP1-Dual™ Cells (NF—KB-SEAP and IRF-Lucia luciferase reporter monocytes, InvivoGen) were used. The reporter cells were knocked-in either of hSTING-WT (R232, #thpd-r232), HAQ variant (#thpd-nfis), or H232 variant (#thpd-h232). All cells were cultured at 37° C. in a humidified incubator at 5% CO₂ and maintained in RPMI 1640 medium (Gibco, #22400097) supplemented with 10% heat-inactivated FBS and 1×Pen-Strep (Gibco, #15140122). To measure STING agonist-mediated IRF3 activity, the cells (9.0×10⁴ cells per well) were seeded in a flat-bottomed 96-well plate. The next day, the cells were treated with compounds in a serial dilution for 24 hours. Subsequently, 20 μL of the cell culture supernatant and 50 μL of the QUANTI-Luc™ reagent (InvivoGen, #rep-qlc) were mixed in a 96-well white plate and then, luciferase activity was measured using an EnVision Xcite multilabel reader. The data were analyzed using Prism8 software (4-parameter).

The potency of STING agonist compounds was evaluated by the activation of Type I Interferon pathway using THP-1 IRF3 reporter assay. Although the activities varied among the STING agonist compounds, half maximal effective concentration (EC₅₀) values for all IRF3 activation were below 1 M and were comparable to Comparative compound #1 or #2, as shown in Table 1 below. In addition, the STING agonist compounds activated human STING variants, such as HAQ and H232, as shown in Table 2. Collectively, these results indicate that the STING agonist compounds strongly stimulate STING signaling pathway.

TABLE 1
STING activity using hSTING-R232 cells

| Compound No. | EC₅₀ (nM) |
|---|---|
| Comparative #1 | 6.8 |
| Comparative #2 | 212.6 |
| 174 | 44.41 |
| 214 | 15.99 |
| 221 | 68.30 |
| 230 | 3.96 |
| 239 | 4.46 |
| 262 | 6.78 |
| 270 | 16.72 |
| 272 | 18.84 |
| 274 | 9.95 |
| 277 | 27.36 |
| 281 | 39.17 |
| 285 | 33.49 |
| 291 | 8.86 |
| 299 | 7.20 |
| 302 | 46.70 |
| 307 | 5.36 |
| 309 | 31.20 |
| 313 | 68.30 |
| 316 | 88.16 |
| 320 | 3.65 |
| 328 | 80.71 |
| 330 | 9.80 |
| 332 | 573.80 |
| 337 | 16.25 |
| 344 | 11.07 |
| 347 | 10.48 |
| 357 | 50.41 |
| 359 | 462.70 |
| 361 | 35.81 |
| 363 | 402.50 |
| 367 | 7.67 |
| 372 | 54.63 |
| 376 | 286.90 |
| 396 | 239.80 |
| 417 | 24.65 |
| 419 | 38.09 |
| 422 | 45.00 |
| 424 | 72.00 |

TABLE 2
STING activity using hSTING-HAQ or H232 cells

| | HAQ | H232 |
|---|---|---|
| Compound No. | EC50 (nM) | EC50 (nM) |
| Comparative #1 | 6.8 | 9.7 |
| Comparative #2 | 212.6 | 36.6 |
| 302 | 83.50 | 96.60 |
| 422 | 476.90 | 119.10 |
| 274 | 14.80 | 48.30 |
| 309 | 52.00 | 70.40 |
| 424 | 516.90 | 116.90 |
| 316 | 399.70 | 250.60 |
| 313 | 115.40 | 216.40 |
| 281 | 108.10 | 96.70 |

Additionally, for analysis of direct binding potency of STING agonist compounds described herein to human STING, HUMAN STING WT BINDING KIT (Cisbio, #64BDSTGPEG) was used according to the manufacture's instruction. It was confirmed that the STING agonist compounds directly bind to human STING protein in vitro, and summary result data were presented as EC₅₀ in Table 3.

TABLE 3
Binding potency

| Compound No. | EC₅₀ (nM) |
|---|---|
| Comparative #1 | 0.55 |
| Comparative #2 | 0.13 |
| 270 | 0.39 |
| 277 | 0.20 |
| 302 | 0.38 |
| 422 | 0.92 |
| 274 | 0.19 |
| 309 | 0.40 |
| 424 | 1.60 |
| 307 | 0.18 |
| 320 | 0.04 |
| 337 | 0.19 |
| 313 | 0.08 |
| 285 | 0.30 |
| 328 | 0.04 |
| 291 | 0.26 |
| 299 | 0.27 |
| 344 | 0.24 |
| 357 | 0.14 |
| 359 | 0.17 |
| 361 | 0.10 |
| 363 | 0.80 |

### Experimental Example 2: Cytokine production induction in vitro assay

For analysis of STING agonist-induced cytokine production, human peripheral blood mononuclear cells (PBMCs, STEMCELL, #70025.3) and human cancer cell lines (BxPC-3 (ATCC, #CRL-1687) and MDA-MB-468 (DSMZ, #ACC 738)) were cultured in RPMI 1640 medium (Gibco, #22400097) supplemented with 10% heat-inactivated FBS and 1×Antibiotic-Antimycotic (Gibco, #15240062). The cells (2.0×10⁴ cells per well) were seeded in a flat-bottomed 96-well plate. PBMCs were stimulated with STING agonist compounds described herein (Compound 214, 221, and 230) at 1 μM for 24 hours. BxPC-3 and MDA-MB-468 tumor cells were treated with STING agonist compounds described herein (Compound 313) in a 5-fold serial dilution from 3 M for 24 hours, respectively. The levels of cytokines in the culture supernatants were measured using the ELISA kit (R&D systems, #DY266, #DY9345-05) according to the manufacture's instruction.

Briefly, each well of a 96-well plate was coated with 100 μL of the diluted capture antibody in PBS and incubated overnight at room temperature (RT). Next day, each well was washed with wash buffer (0.05% Tween 20 in PBS) three times using an auto-washer. The plates were blocked with 300 μL of Reagent Diluent (1% BSA in PBS) for 1 hour at RT. The aspiration/wash step was repeated. 100 μL of diluted samples or standards in Reagent Diluent was added to each well and incubated for 2 hours at RT. After wash, 100 μL of the Detection Antibody pre-diluted in Reagent Diluent was added into each well and incubated for 2 hours at RT. After wash, 100 μL of the working diluted Streptavidin-TRP in Reagent Diluent was added and incubated under subdued light conditions for 20 minutes, followed by repeated aspiration/wash. 100 μL of substrate solution (mixed H₂O₂ and Tetramethylbenzidine in a 1:1 ratio) was added and incubated for 20 minutes at RT in avoiding direct light exposure. The reaction was stopped by adding 50 μL of stop solution (2N H₂SO₄). The optical density was determined using a microplate reader (Molecular Devices VersaMax Microplate Reader). The optical density (O.D.) of each well was normalized by subtracting the reading at 570 nm from the reading at 450 nm.

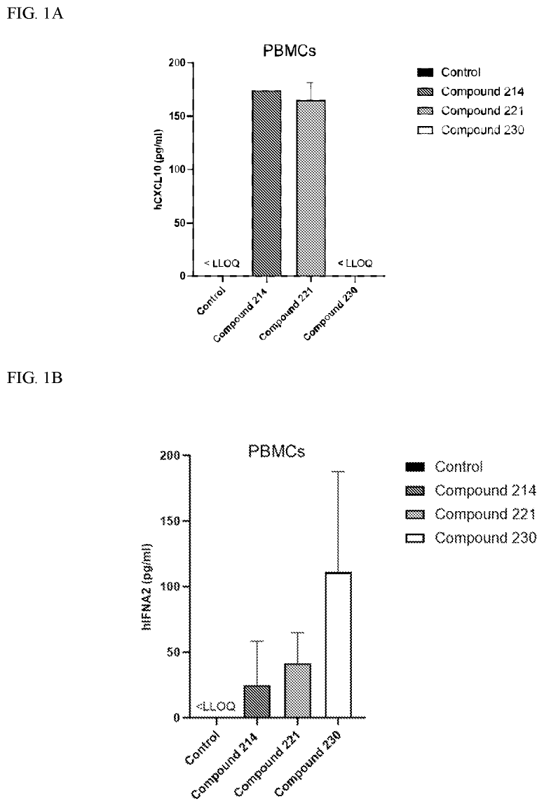

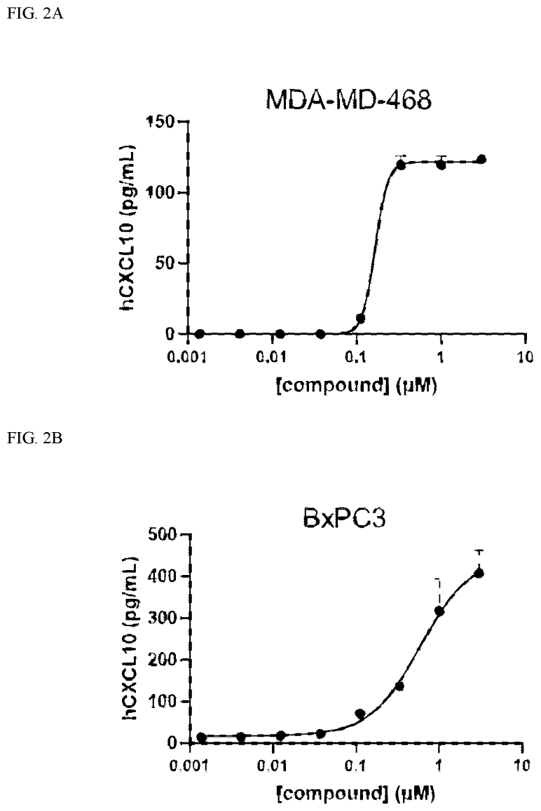

Since the STING is expressed in both tumor and immune cells, the STING agonist compounds induced cytokine production through activation of STING pathway in those cells. Specifically, as shown in FIGS. 1 and 2, the STING agonist compounds strongly mediated the production of human CXCL-10 and human IFNα, which are key downstream cytokines of STING pathway.

### Experimental Example 3: Immune Cells Activation In Vitro Assay

To test the effect of STING agonist compounds described herein in the activation of innate and adaptive immune cells, flow cytometry (FACS) was performed. Human THP-1 cells (Korean Cell Line Bank, #40202) and PBMCs were seeded at 1.2×10⁵ cells per well in a round-bottomed 96-well plate in RPMI 1640 medium (Gibco, #22400097) supplemented with 10% heat-inactivated FBS and 1× Antibiotic-Antimycotic (Gibco, #15240062) and rested for 24 hours at 37° C. The cells were stimulated with the STING agonist compounds as described; For THP-1 stimulation, the cells were treated with the STING agonist compound (Compound 313) in a 5-fold serial dilution from 1 μM for 48 hours. The induction of cell surface activation markers, CD86 (co-stimulatory molecule) and HLA-DR (MHC-class II), was measured by flow cytometry. For PBMCs, the cells were stimulated with the STING agonist compounds, Compound 274 and 277 at 100 nM for 48 hours or Compound 344 and 361 at 3 μM for 24 hours, respectively. For flow cytometry analysis, the stimulated cells were centrifuged at 300 g for 5 minutes at 4° C., and then resuspended in FACS buffer (2% FBS, 2 mM EDTA in PBS). After staining cells with human Fc blocker (BD Biosciences, #564220) in FACS buffer for 10 minutes at 4° C. Subsequently, the cells were stained with fluorescence-conjugated antibodies against CD8, CD3, CD56, and CD69 for CD8 T and NK cells activation and against CD86 and MHC-II for THP-1 naïve cells activation for 40 minutes at 4° C. After washing three times with FACS buffer, the cells were resuspended in FACS buffer and analyzed by FACSlyric (BD bioscience). The data were analyzed with Flowjo 9.0 software (BD bioscience).

TABLE 4
Antibody used in FACS

| | | | Dilu- | | |
|---|---|---|---|---|---|
| Target | Clone | Fluorescence | tion | Producer | Cat |
| CD3 | SK7 | PE-Cy7 | 1:50 | BD biosciences | 557851 |
| CD8 | SK1 | Alexafluor647 | 1:50 | BD biosciences | 560176 |
| CD56 | NCAM-1 | PE | 1:50 | BD biosciences | 562432 |
| CD69 | FN50 | BV421 | 1:50 | BD biosciences | 562884 |
| CD86 | 2331(FUN- | APC | 1:50 | BD biosciences | 555660 |
| | 1) | | | | |
| HLA- | G46-6 | FITC | 1:50 | BD biosciences | 555811 |
| DR | | | | | |

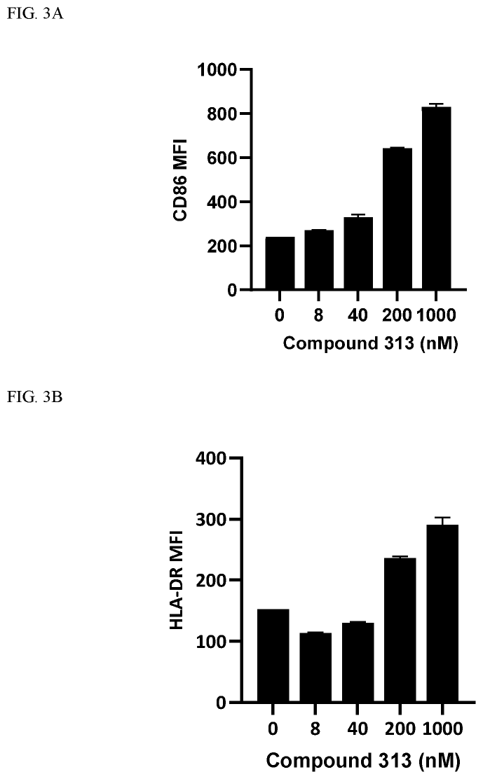

As macrophages and monocytes express high levels of STING protein, the human monocytic cell line THP-1 also shows high STING expression. As shown in FIGS. 3A and 3B, when THP-1 was treated with the STING agonist compounds, co-stimulatory molecule CD86 and MHC class II molecule HLA-DR were up-regulated on cell surface in a dose-dependent manner. These suggest that the STING agonist compounds activate myeloid cells, such as macrophages and dendritic cells, and may enhance their tumor antigen presentation through up-regulation of the antigen presentation machinery.

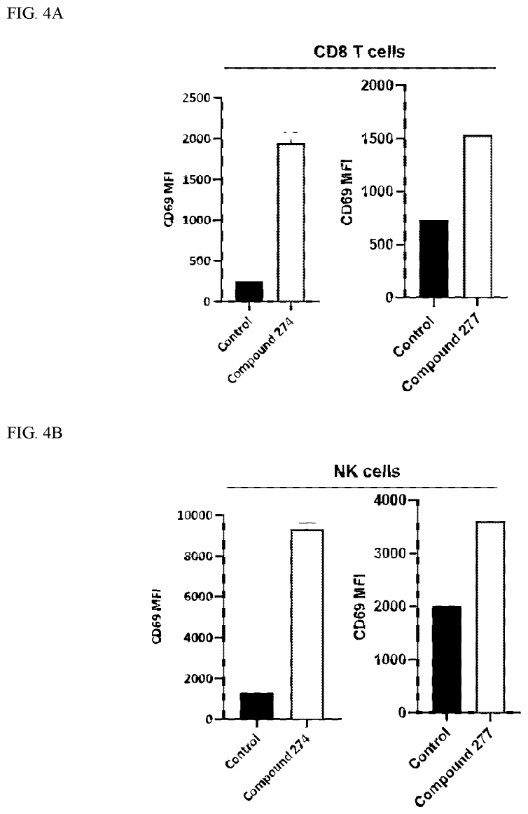

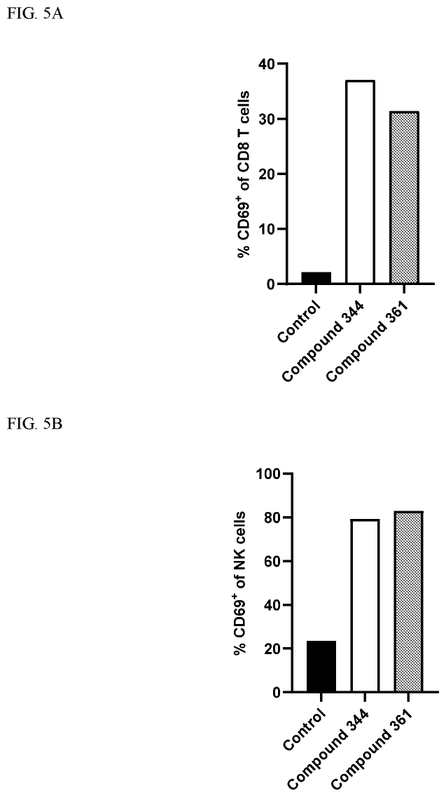

After treating human PBMCs with the STING agonist compounds, it was analyzed the activation of CD8⁺ T cells and NK cells by measuring CD69, an activation marker, on their cell surface by flow cytometry. The STING agonist compounds greatly enhanced CD69 expression on both CD8⁺ T and NK cells (FIGS. 4A and 4B), and the expansion of those activated cells (FIGS. 5A and 5B).

Thus, the results suggest that the STING agonist compounds effectively induce innate and adaptive immune cell responses.

### Experimental Example 4: Assessment of Pharmacokinetics

For in vivo evaluation of STING agonist compounds described herein in naïve Balb/C mouse, single doses of STING agonist compounds were given at 1.5 mg/kg intravenously into female BALB/c mice (Orientbio, South Korea) at 6-8 weeks of age. Pharmacokinetics were studied following injection of the STING agonist compounds into Balb/C mice. Plasma samples were taken at various time points and stored frozen for analysis. The plasma concentrations of the STING agonist compounds at the indicated time points were measured using a LC-MS/MS analysis method.

Briefly, 250 μL of acetonitrile (ACN) solution was added in both 50 μL of sample and 50 μL of plasma containing 10 nM Dextromethorphan (internal standard), and the solutions were mixed vigorously using a vortex mixer for 5 minutes. The samples were then spun down at 14,000 rpm, 4° C. for 5 minutes. 100 μL of supernatants were combined with 100 μL of mobile phase A (0.1% formic acid in water with 5% ACN) and mixed thoroughly. The samples were measured for the STING agonist compounds using a LC-MS/MS (Nexera LC40 (SHIMADZU) and QTRAP4500 (SCIEX)).

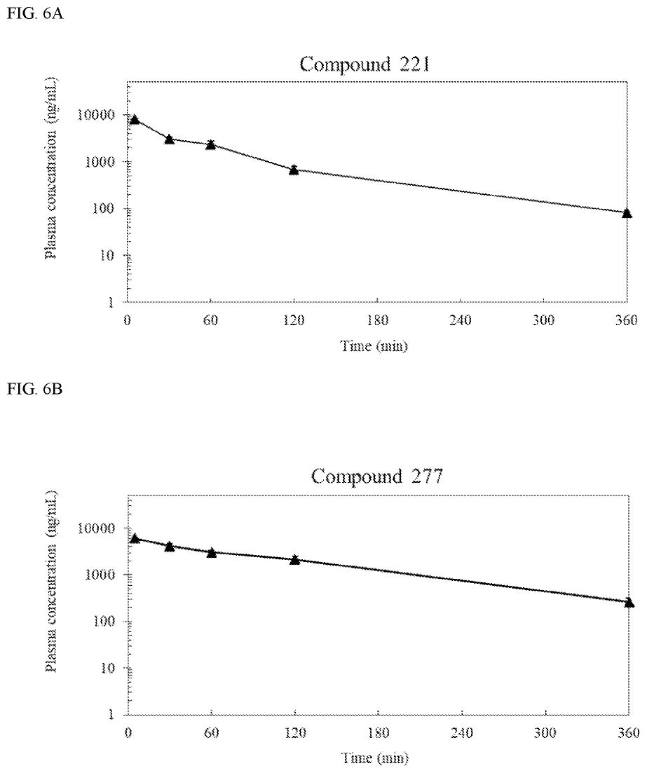

The PK profiles of Compound 221, 277, 281, 274, Comparative #1 and #2 were summarized in Tables 5 to 10. Graphical representation of the plasma PK was shown in FIGS. 6A to 6D. Compared to the Comparative #1 and 2, the STING agonist compounds showed significantly stable pharmacokinetic profiles in mice.

TABLE 5
Compound 221

| | 1.5 mg/kg |
|---|---|
| Mouse (Balb/c) | single IV, n = 3 |
| AUCₗₐₛₜ | 6.67 ± 0.22 |
| (μg*h/mL) | |
| AUC_{∞} μg*h/mL) | 6.80 ± 0.23 |
| AUC_{∞, norm} | 4.53 ± 0.15 |
| (kg*h/L) | |
| CL (L/h/kg) | 0.22 ± 0.01 |
| V_{SS} (L/kg) | 0.26 ± 0.01 |
| Cₘₐₓ (μg/mL) | 8.06 ± 0.45 |
| C_{max, norm} (kg/L) | 5.37 ± 0.30 |
| C₀ (μg/mL) | 9.79 ± 0.43 |
| t_{1/2} (h) | 1.06 ± 0.04 |
| F (%) | (100) |

TABLE 6
Compound 277

| | 1.5 mg/kg |
|---|---|
| Mouse (Balb/c) | single IV, n = 3 |
| AUCₗₐₛₜ | 10.5 ± 0.49 |
| (μg*h/mL) | |
| AUC_{∞} μg*h/mL) | 11.1 ± 0.37 |
| AUC_{∞, norm} | 7.37 ± 0.25 |
| (kg*h/L) | |
| CL (L/h/kg) | 0.14 ± 0.00 |
| V_{SS} (L/kg) | 0.27 ± 0.03 |
| Cₘₐₓ (μg/mL) | 6.06 ± 0.36 |
| C_{max, norm} (kg/L) | 4.04 ± 0.24 |
| C₀ (μg/mL) | 6.53 ± 0.32 |
| t_{1/2} (h) | 1.38 ± 0.19 |
| F (%) | (100) |

TABLE 7
Compound 281

| | 1.5 mg/kg |
|---|---|
| Mouse (Balb/c) | single IV, n = 3 |
| AUCₗₐₛₜ | 14.8 ± 1.35 |
| (μg*h/mL) | |
| AUC_{∞} μg*h/mL) | 14.8 ± 1.34 |
| AUC_{∞, norm} | 9.89 ± 0.90 |
| (kg*h/L) | |
| CL (L/h/kg) | 0.10 ± 0.01 |
| V_{SS} (L/kg) | 0.32 ± 0.03 |
| Cₘₐₓ (μg/mL) | 6.16 ± 0.42 |
| C_{max, norm} (kg/L) | 4.11 ± 0.28 |
| C₀ (μg/mL) | 6.50 ± 0.49 |
| t_{1/2} (h) | 2.80 ± 0.27 |
| F (%) | (100) |

TABLE 8
Compound 274

| | 1.5 mg/kg |
|---|---|
| Mouse (Balb/c) | single IV, n = 3 |
| AUCₗₐₛₜ | 56.9 ± 4.12 |
| (μg*h/mL) | |
| AUC_{∞} μg*h/mL) | 57.1 ± 3.90 |
| AUC_{∞, norm} | 38.1 ± 2.60 |
| (kg*h/L) | |
| CL (L/h/kg) | 0.03 ± 0.00 |
| V_{SS} (L/kg) | 0.17 ± 0.01 |
| Cₘₐₓ (μg/mL) | 9.65 ± 0.61 |
| C_{max, norm} (kg/L) | 6.44 ± 0.41 |
| C₀ (μg/mL) | 10.0 ± 0.64 |
| t_{1/2} (h) | 4.26 ± 0.74 |
| F (%) | (100) |

TABLE 9
Comparative #1

| | 1.5 mg/kg |
|---|---|
| Mouse (Balb/c) | single IV, n = 3 |
| AUCₗₐₛₜ | 0.98 ± 0.10 |
| (μg*h/mL) | |
| AUC_{∞} μg*h/mL) | 0.99 ± 0.10 |
| AUC_{∞, norm} | 0.66 ± 0.07 |
| (kg*h/L) | |
| CL (L/h/kg) | 1.53 ± 0.16 |
| V_{SS} (L/kg) | 0.70 ± 0.15 |
| Cₘₐₓ (μg/mL) | 2.91 ± 0.45 |
| C_{max, norm} (kg/L) | 1.94 ± 0.30 |
| C₀ (μg/mL) | 4.63 ± 0.86 |
| t_{1/2} (h) | 0.91 ± 0.04 |
| F (%) | (100) |

TABLE 10
Comparative #2

| | 1.5 mg/kg |
|---|---|
| Mouse (Balb/c) | single IV, n = 3 |
| AUCₗₐₛₜ | 0.07 ± 0.01 |
| (μg*h/mL) | |
| AUC_{∞} μg*h/mL) | 0.08 ± 0.01 |
| AUC_{∞, norm} | 0.05 ± 0.01 |
| (kg*h/L) | |
| CL (L/h/kg) | 19.7 ± 2.59 |
| V_{SS} (L/kg) | 5.10 ± 1.01 |
| Cₘₐₓ (μg/mL) | 0.24 ± 0.03 |
| C_{max, norm} (kg/L) | 0.16 ± 0.02 |
| C₀ (μg/mL) | 0.38 ± 0.05 |
| t_{1/2} (h) | 0.20 ± 0.03 |
| F (%) | (100) |

### Experimental Example 5: In Vitro Assessment of Normal Cell Cytotoxicity

PBMCs were also purchased from STEMCELL™ (#700025.2). The cells (8.0×10⁴ cells per well) were seeded in a flat-bottomed 96-well plate in RPMI 1640 medium (Gibco, #22400097) with 10% heat-inactivated FBS and 1× Antibiotic-Antimycotic (Gibco, #15240062) and rested for 24 hours at 37° C. The cells were treated with the STING agonist compounds described herein (Compound 277, 302, 422, 274, 309, 424, 316, 313, 281, Comparative #1 and #2) in a serial dilution. After 72 hours, the cell viability was measured using CellTiter-Glo Luminescent Cell Viability Assay (Promega, #G7573) according to the manufacture's instruction. The signal was detected using an EnVision Xcite multilabel reader and data were analyzed using GraphPad Prism 8 software.

To verify the effect of the STING agonist compounds in normal cell cytotoxicity, the PBMCs were treated with the STING agonist compounds for 3 days and measured the percentage of cell death. As shown in 50% cytotoxic concentration (CC₅₀) values in Table 11, compared to the Comparative #1 and #2, all STING agonist compounds had higher CC₅₀ values, implying lower cytotoxicity in normal immune cells.

TABLE 11
In vitro cell cytotoxicity in PBMCs

| Compound No. | CC₅₀ (nM) |
|---|---|
| Comparative #1 | 3.34 |
| Comparative #2 | 6.28 |
| 277 | 32.11 |
| 302 | 59.21 |
| 422 | 207.3 |
| 274 | 16.02 |
| 309 | 44.31 |
| 424 | 147.2 |
| 316 | 206.9 |
| 313 | 98.03 |
| 281 | 126.6 |

The CD34⁺ hematopoietic stem cells (HSCs) were purchased from STEMCELL™ (#70002.3). The cells (2×10⁴ cells per well) were cultured in StemSpan™ SFEM II medium supplemented with StemSpan™ CD34+ Expansion Supplement (#02691) in a 6-well plate at 37° C. for 7 days. On day 3 or 4, an equal volume of fresh complete medium was added into the cell culture. On day 7, the cells (4×10⁴ cells per well) were seeded in a 96-well white plate and rested for 24 hours in the same condition.

To verify the effect of STING agonist compounds described herein in normal cell cytotoxicity, the HSCs were treated with STING agonist compounds described herein (Compound 277, 302, 422, 424, 313, 363, 396 and Comparative #1) in a serial dilution. After 72 hours, the cell viability was measured using CellTiter-Glo Luminescent Cell Viability Assay (Promega, #G7573) according to the manufacture's instruction. The signal was detected using an EnVision Xcite multilabel reader and data were analyzed using GraphPad Prism 8 software. As shown in 50% cytotoxic concentration (CC₅₀) values in Table 12, compared to the Comparative #1, all STING agonist compounds had higher CC₅₀ values, implying lower cytotoxicity in HSCs.

TABLE 12
In vitro cell cytotoxicity in CD34⁺ HSC

| Compound No. | CC₅₀ (nM) |
|---|---|
| Comparative #1 | 2.10 |
| 277 | 13.50 |
| 302 | 21.50 |
| 422 | 32.10 |
| 424 | 30.60 |
| 313 | 44.50 |
| 363 | 86.10 |
| 396 | 156.10 |

### Experimental Example 6: In Vivo Efficacy in Syngeneic Mouse Models

Female BALB/c mice (KOTECH, South Korea) with 6 weeks of age, were used for all studies that were completed under the approval of the Legochembio Science's Institutional Animal Care and Use Committee (IACUC). CT26 or EMT6 cells (American Type Culture Collection (ATCC), #CRL-2638, #CRL-2755) were maintained in RPMI 1640 medium (Gibco, #22400097) supplemented with 10% heat-inactivated FBS and 1× Antibiotic-Antimycotic (Gibco, #15240062) at 37° C. with 5% CO₂. The mycoplasma-negative cells were used for all experiments and mycoplasma test was done regularly using e-Myco™ VALiD Mycoplasma PCR Detection Kit (iNtRON biotechnology, #25239). CT26 cells (2×10⁵ cells/mouse) or EMT6 cells (5×10⁵ cells/mouse) in PBS were implanted subcutaneously into the shaved right flank. Tumor volumes and body weights were measured every 3 to 4 days, and the tomor volumes were calculated according to the formula 0.5×(length)×(width)².

Specifically, to determine in vivo efficacy of STING agonist compounds (Compound 214 and 221) in CT26 syngeneic mouse model, when tumor volume reached 70 mm³, each compound was given at 1.5 mg/kg intravenously every 3 or 4 days (total three times). To determine in vivo efficacy of STING agonist compound (Compound 274) in CT26 syngeneic mouse model, when tumor volume 55 mm³, the compound was given at a variety of doses intravenously once weekly for 3 weeks except a single dose of 1 mg/kg. To compare in vivo efficacy of STING agonist compounds (Compound 274 and 281) in CT26 syngeneic mouse model to the Comparative Compound #1, when tumor volume 55 mm³, each compound was given at 0.5 mg/kg intravenously once weekly for three weeks. To compare in vivo efficacy of STING agonist compounds (Compound 274, 313, 344 and 396) in CT26 syngeneic mouse model to the Comparative Compound #3, when tumor volume 80 mm³, each compound was given at 0.3 mg/kg intravenously once weekly for 3 weeks. To compare in vivo efficacy of STING agonist compounds (Compound 274 and 313) in EMT6 syngeneic mouse model to the Comparative Compounds #1 and #2, when tumor volume 100 mm³, each compound was given at 0.125 mg/kg intravenously once weekly for three weeks. To determine in vivo efficacy of STING agonist compound (Compound 313) in EMT6 syngeneic mouse model, when tumor volume 100 mm³, the compound was given at the indicated doses intravenously once weekly for 3 weeks except for the dose at 0.05 mg/kg twice a week.

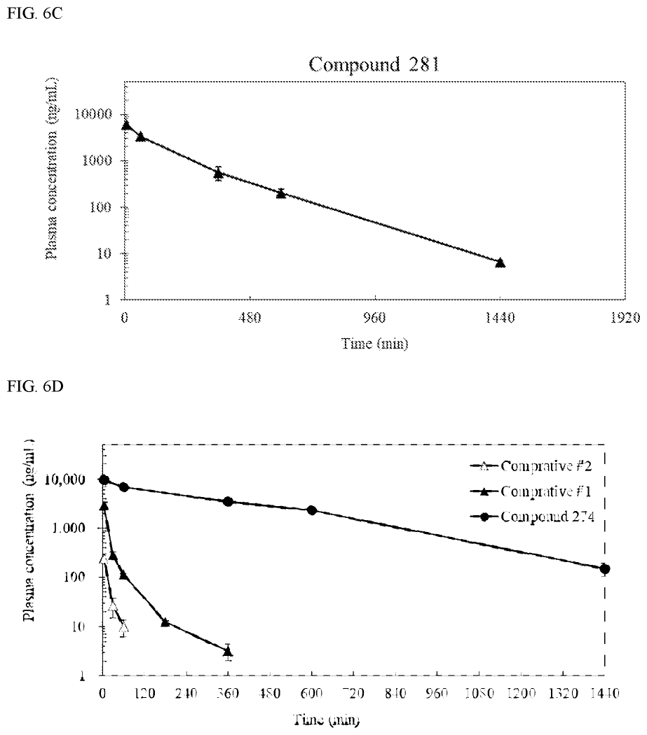

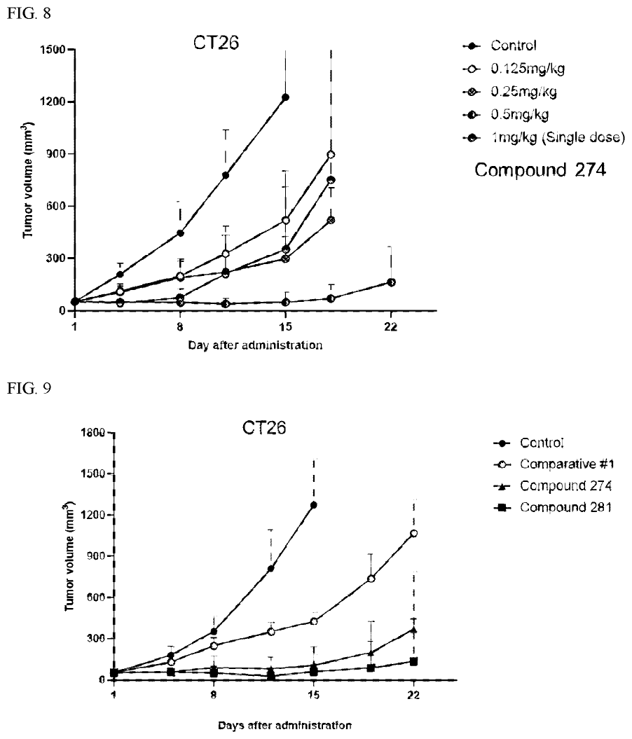

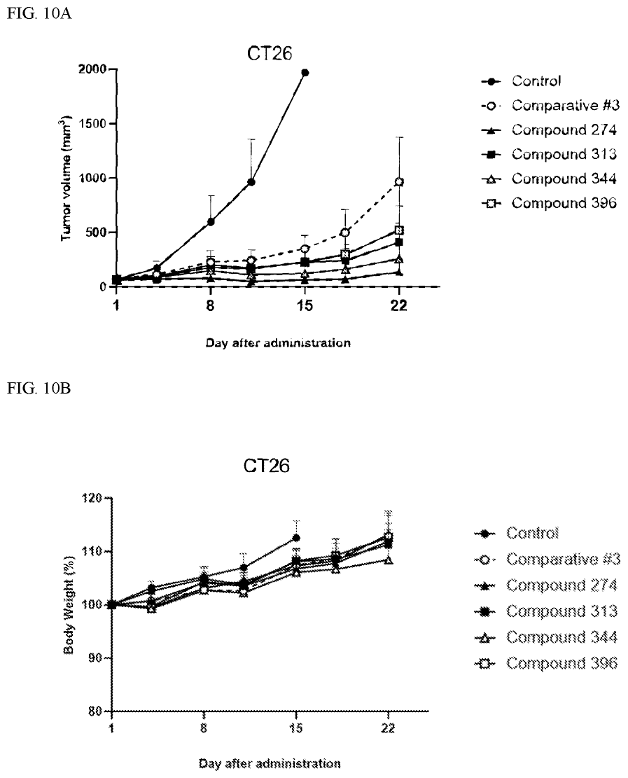

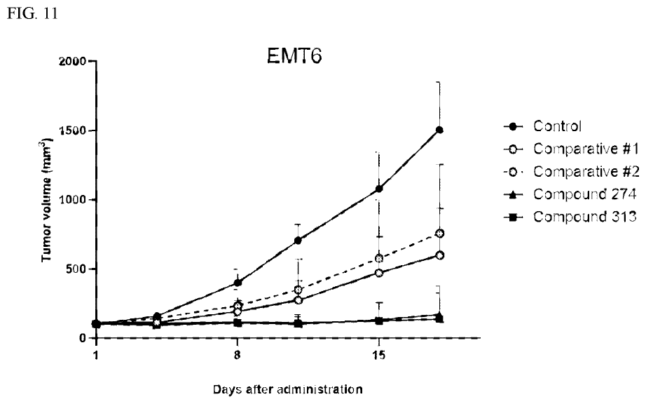

A syngeneic system was used to assess the ability of the STING agonist compounds to induce immune responses and drive an anti-tumor immune response. In both CT26 and EMT6 syngeneic models, the STING agonist compounds remarkably controlled tumor growth at various doses (FIGS. 7 to 12).

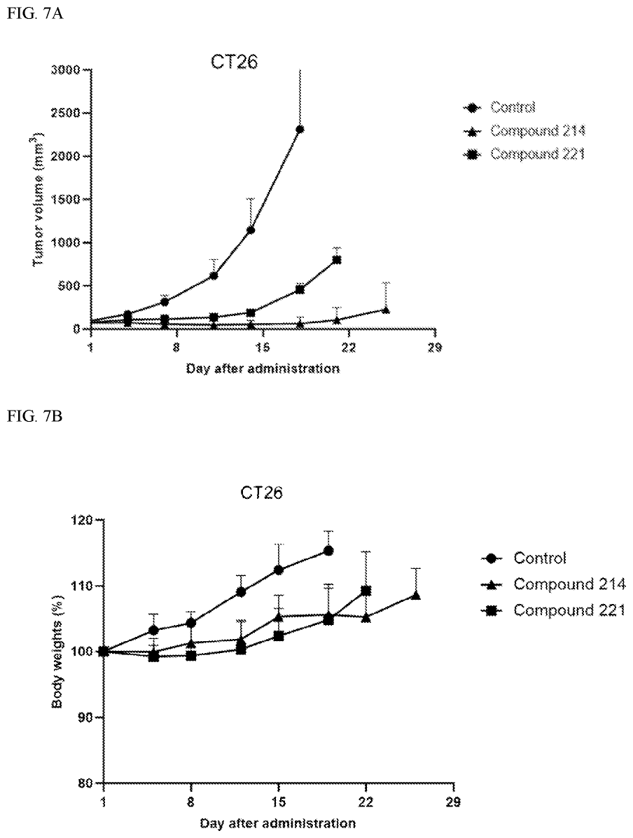

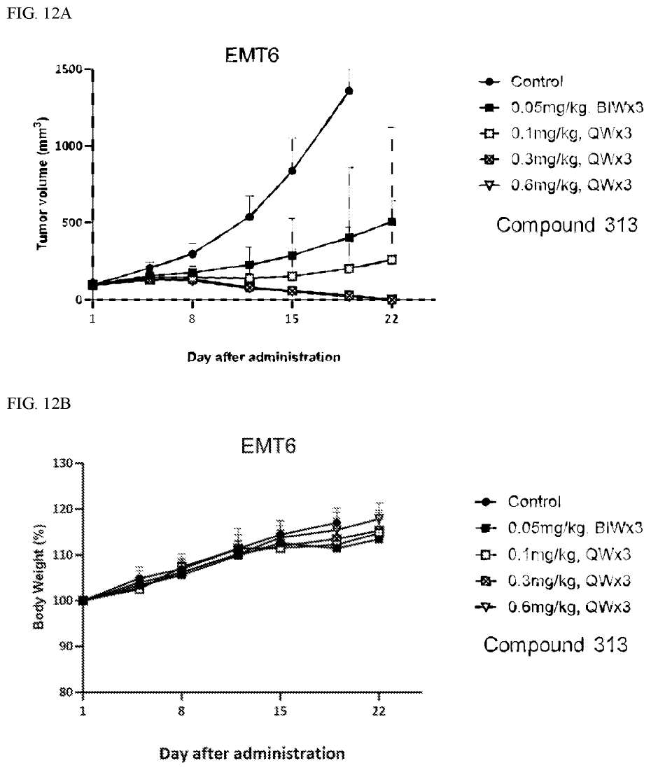

Particularly, Compound 274 completely eradicated tumors in a group of 0.5 mg/kg (2 out of 5 mice) and in single-dose group of 1 mg/kg (2 out of 5 mice) in CT26 model (FIG. 8). Compound 313 also eliminated tumors in all groups (1 out of 5 for 0.05 mg/kg, 2 out of 5 for 0.1 mg/kg, 5 out of 5 for both 0.3 and 0.6 mg/kg) in EMT6 model (FIG. 12).

Consistent with in vitro effectiveness, the STING agonist compounds described herein showed the superior in vivo efficacy in different syngeneic mouse models.

Taken together, the STING agonist compounds described herein have strongly competitive profiles of high anti-tumor activity, but low toxicity.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound represented by structural formula (I), or a pharmaceutically acceptable salt thereof: 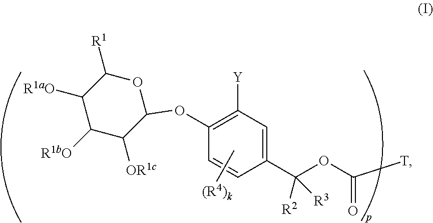
wherein:
T is a moiety comprising a Stimulator of Interferon Genes (STING) agonist,
p is 1 or 2,
each instance of R¹ is independently CH₂OR¹¹ or COOR¹²,
each instance of R^{1a}, R^{1b}, R^{1c}, and R¹¹ is independently H or a hydroxyl protecting group,
each instance of R¹² is independently H or a carboxyl protecting group,
each instance of R² and R³ is independently H or alkyl, or R² and R³ together with a carbon atom to which they are attached form a cycloalkyl,
each instance of R⁴ is independently selected from halogen, alkyl, CN, and NO₂,
each instance of k is independently 0, 1, 2, or 3,
each instance of Y is independently selected from H, —C(O)NHL^{u}U, —C(O)NR′(L^{u}U), —C(O)N(L^{u}U)₂ and —C(O)OH,
each instance of L^{u} is a first linker,
each instance of U is independently selected from H, alkyl, amino, azido, acetylenyl, alkylamino, heterocyclyl, alkoxy, —COOH, —P(O)(OH)₂, —OH, -DBCO and a saccharide, and
each instance R′ is independently selected from alkyl, cycloalkyl, alkoxy, alkylthio, mono- or di-alkylamino, heteroaryl, and aryl.

2. The compound of claim 1, wherein the compound of formula (I) is a compound of formula (Ia):

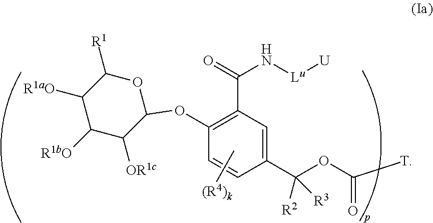

3. The compound of claim 1 or 2, wherein p is 1 and T is a moiety represented by one of the following structural formulas: 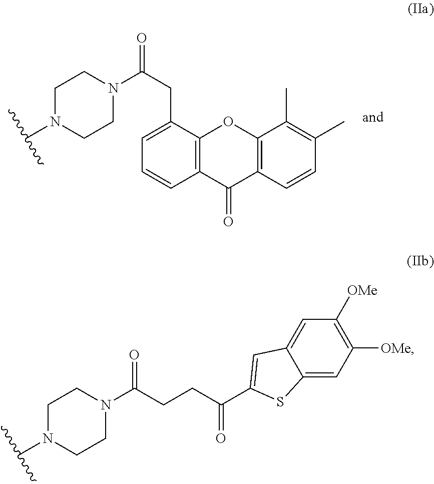 [SMILES: *N1CCN(C(=O)Cc2cccc3c(=O)c4ccc(C)c(C)c4oc23)CC1 ; *N1CCN(C(=O)CCC(=O)c2cc3cc(OC)c(OC)cc3s2)CC1]
wherein 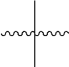
is the point of connection to the —C(O)O(CR²R³)— fragment of the compound represented by structural formula (I).

4. The compound of claim 1 or 2, wherein, T is a moiety represented by Formula (IIc): 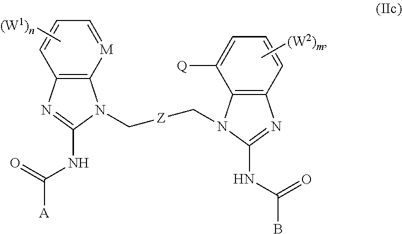
wherein:
T is coupled to the —C(O)OCR²R³— fragment of formula (I) via W¹, W², A, B, or L²,
M is N, C(X^{a}R^{a}) or C(X^{b}L¹L²-),
Q is —X^{a}R^{a} or -X^{b}L¹L²-,
each instance of W¹ and W² is independently selected from alkyl, amino, amido, carboxylic acid, ester, and hydrazido, e.g. —C(O)NH-alkyl-N(alkyl)- or —C(O)NH—N(alkyl)-;
n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino and —O(alkylene)guanidino,
L¹ is selected from alkylene, heteroalkylene, alkenylene, alkynylene, cycloalkylene, heterocyclylene, arylene, and heteroarylene,
L² is a bond, or a linker moiety coupled to L¹ and comprising a nitrogen atom coupled to the —C(O)O(CR²R³)— fragment of the compound represented by structural formula (I).

5. The compound of claim 4, wherein X^{a} is O.

6. The compound of claim 4 or 5, wherein R^{a} is selected from C₁₋₆ alkyl, heterocyclylalkyl, cycloalkylalkyl, -(alkylene)carboxylic acid, and -(alkylene)guanidino.

7. The compound of any one of claims 4-6, wherein L² is selected from a bond, 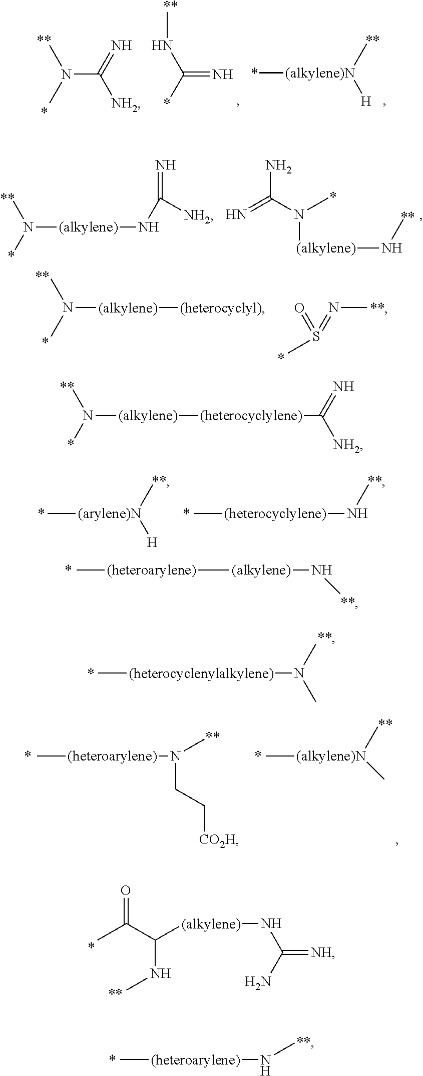
and *heterocyclylene**, and wherein * is the point of connection to L¹ and ** is the point of connection to the —C(O)O(CR²R³)— fragment of the compound represented by structural formula (I).

8. The compound of claim 3, wherein T is represented by structural formula (IIa).

9. The compound of claim 8, wherein the compound is

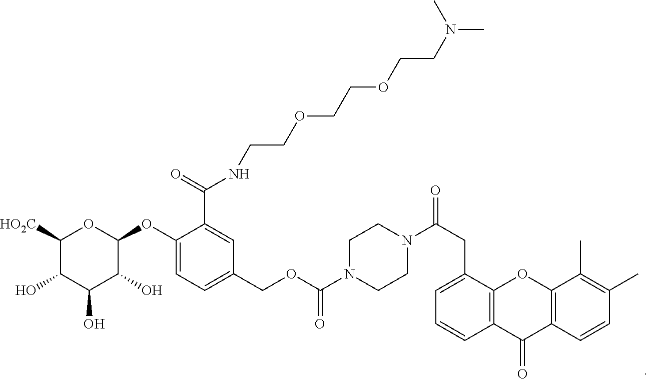 [SMILES: Cc1ccc2c(=O)c3cccc(CC(=O)N4CCN(C(=O)OCc5ccc(O[C@@H]6O[C@H](C(=O)O)[C@@H](O)[C@H](O)[C@H]6O)c(C(=O)NCCOCCOCCN(C)C)c5)CC4)c3oc2c1C]

10. The compound of claim 3, wherein T is represented by structural formula (IIb).

11. The compound of claim 10, wherein the compound is

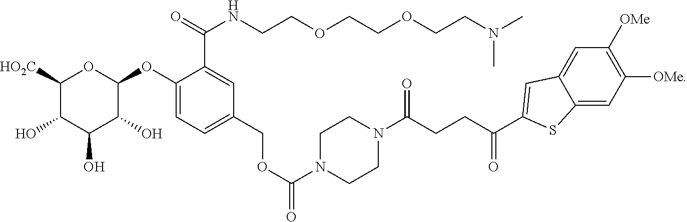 [SMILES: COc1cc2cc(C(=O)CCC(=O)N3CCN(C(=O)OCc4ccc(O[C@@H]5O[C@H](C(=O)O)[C@@H](O)[C@H](O)[C@H]5O)c(C(=O)NCCOCCOCCN(C)C)c4)CC3)sc2cc1OC]

12. The compound of any one of claims 4-6, wherein T is coupled to the —C(O)OCR²R³-fragment of the compound represented by structural formula (I) via W², and T is a moiety represented by formula (IIc1): 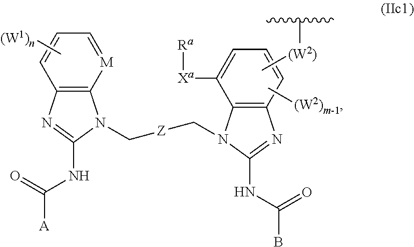
wherein 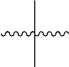
is the point of connection to the —C(O)O(CR²R³)— fragment of the compound represented by structural formula (I).

13. The compound of any one of claims 4-6, wherein T is coupled to the —C(O)O(CR²R³)— fragment of the compound represented by structural formula (I) via B, and T is a moiety represented by formula (IIc2): 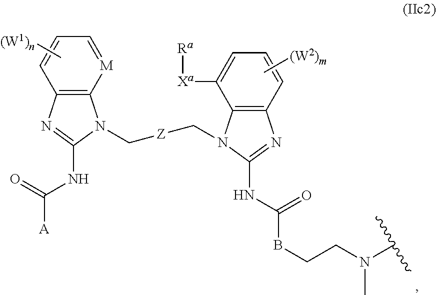
wherein 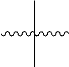
is the point of connection to the —C(O)O(CR²R³)— fragment of the compound represented by structural formula (I).

14. The compound of any one of claims 4-6, wherein T is coupled to the —C(O)O(CR²R³)— fragment of the compound represented by structural formula (I) via L², and T is a moiety represented by formula (IIc3): 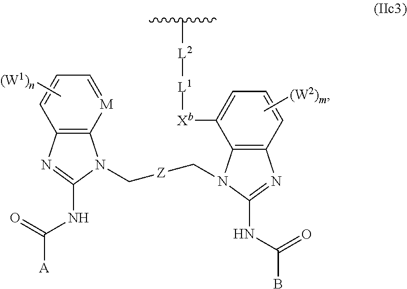
wherein 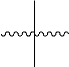
is the point of connection to the —C(O)O(CR²R³)— fragment of the compound represented by structural formula (I).

15. The compound of any one of claims 4-6, wherein T is coupled to the —C(O)O(CR²R³)— fragment of the compound represented by structural formula (I) via L², and T is a moiety represented by formula (IIc4): 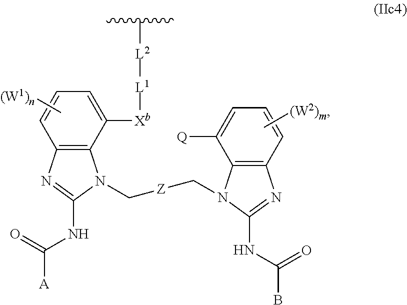
wherein 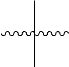
is the point of connection to the —C(O)O(CR²R³)— fragment of the compound represented by structural formula (I).

16. The compound of any one of claim 14 or 15, wherein L² is selected from a bond, 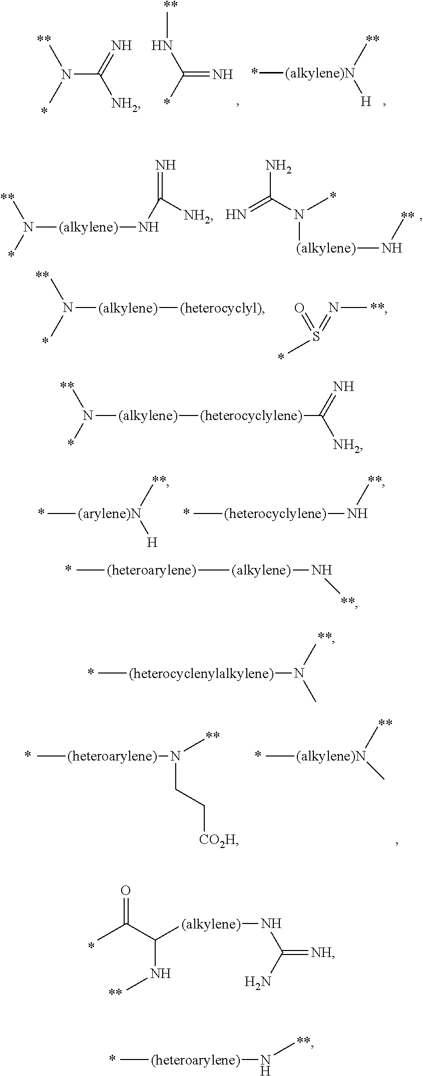
and *heterocyclylene**, and wherein * is the point of connection to L¹ and ** is the point of connection to the —C(O)O(CR²R³)— fragment of the compound represented by structural formula (I).

17. The compound of claim 14 or 15, wherein L² is a second linker comprising ^{#}OC(O)NR⁵-L⁴-NR⁶, ^{#}OC(O)-L⁴-NR⁶, or ^{#}OC(O)NR⁵-L⁴-(heterocyclylene), wherein:
the heterocyclylene comprises a nitrogen atom connected to the —C(O)O(CR²R³)— fragment of the compound represented by structural formula (I), and wherein # is the point of connection to L¹,
each instance of L⁴ is independently alkylene or arylalkylene, and
each instance of R⁵ is independently selected from H, alkyl, and dialkylaminoalkyl, and
each instance of R⁶ is independently selected from H, alkyl, and dialkylaminoalkyl.

18. The compound of claim 17, wherein L² is selected from 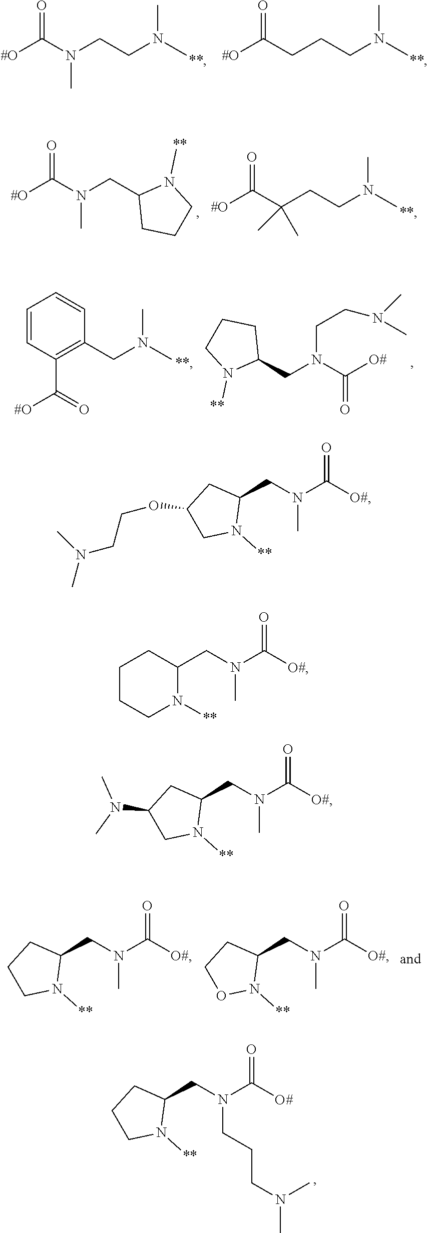
wherein ** indicates the connection point to the —C(O)O(CR R³)— fragment of the compound represented by structural formula (I).

19. The compound of claim 18, wherein L² is selected from

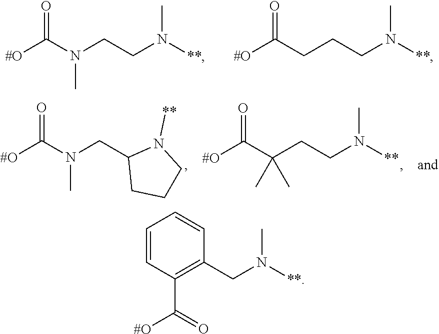

20. The compound of claim 19, wherein L² is

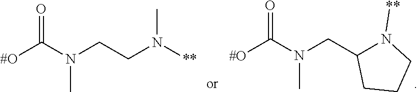

21. The compound of claim 1, wherein p is 1 and T is a moiety represented by formula (IIc5): 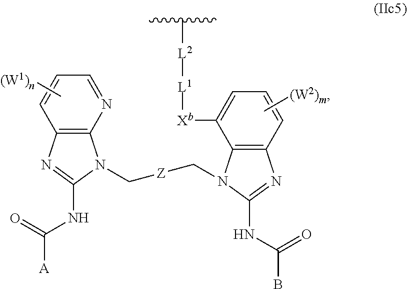
wherein:
T is coupled to the —C(O)O(CR²R³)— fragment of the compound represented by structural Formula (I) via L²,
each instance of W¹ and W² is independently selected from alkyl, amino, amido, carboxylic acid, ester, and hydrazido,
n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino and —O(alkylene)guanidino,
L¹ is selected from alkylene, heteroalkylene, alkenylene, alkynylene, cycloalkylene, heterocyclylene, arylene, and heteroarylene,
L² is a bond, a linker moiety coupled to L¹ and comprising a nitrogen atom coupled to the —C(O)O(CR²R³)— fragment of the compound represented by structural formula (I), or a second linker,
wherein 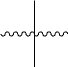
is the point of connection to the —C(O)O(CR²R³)— fragment of the compound represented by structural formula (I).

22. The compound of claim 21, wherein L² is a second linker comprising ^{#}OC(O)NR⁵-L⁴-NR⁶, ^{#}OC(O)-L⁴-NR⁶, or ^{#}OC(O)NR⁵-L⁴-(heterocyclylene), wherein:
the heterocyclylene comprises a nitrogen atom connected to the —C(O)O(CR²R³)— fragment of the compound represented by structural formula (I), and wherein # is the point of connection to L¹,
each instance of L⁴ is independently alkylene or arylalkylene,
each instance of R⁵ is independently selected from H, alkyl, and dialkylaminoalkyl, and
each instance of R⁶ is independently selected from H, alkyl, and dialkylaminoalkyl.

23. The compound of claim 1 or 2, wherein p is 2 and T is a moiety represented by one of the following structural formulas: 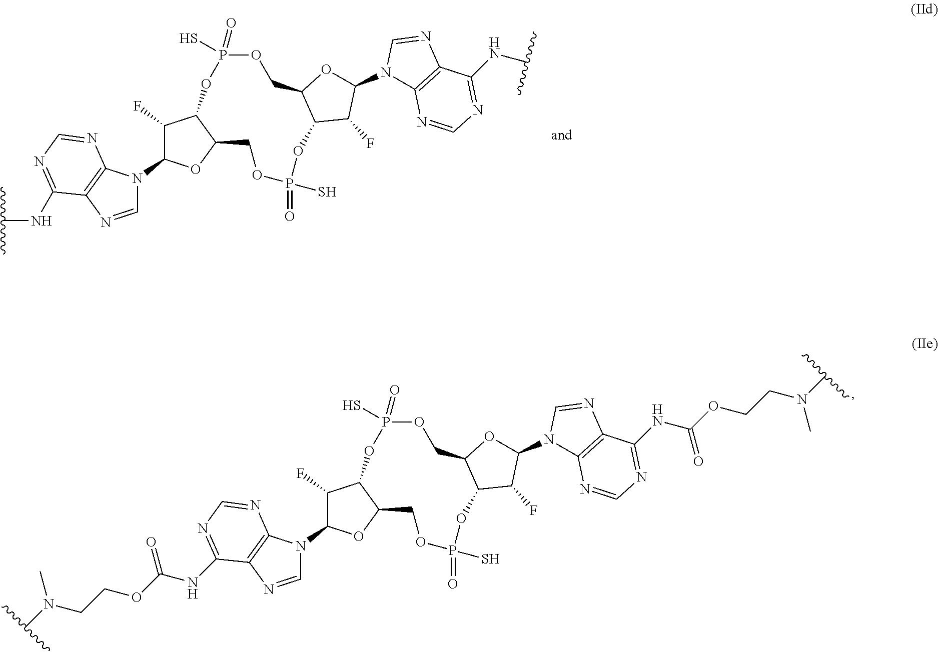 [SMILES: *Nc1ncnc2c1ncn2[C@@H]1O[C@@H]2COP(=O)(S)O[C@H]3[C@@H](F)[C@H](n4cnc5c(N*)ncnc54)O[C@@H]3COP(=O)(S)O[C@H]2[C@H]1F ; *N(C)CCOC(=O)Nc1ncnc2c1ncn2[C@@H]1O[C@@H]2COP(=O)(S)O[C@H]3[C@@H](F)[C@H](n4cnc5c(NC(=O)OCCN(*)C)ncnc54)O[C@@H]3COP(=O)(S)O[C@H]2[C@H]1F]
wherein each 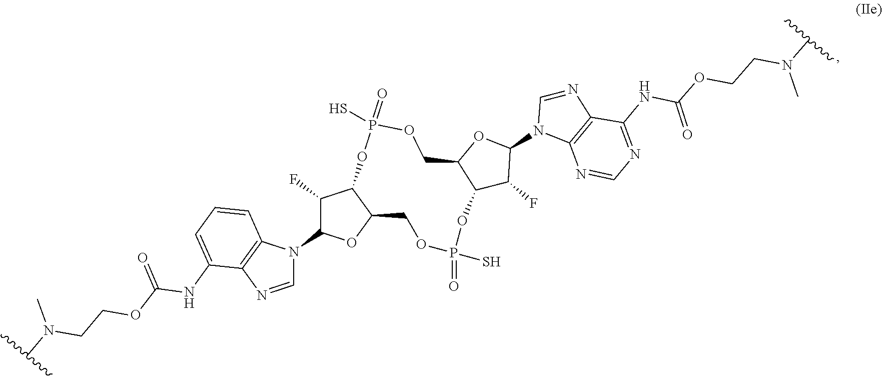
is a point of connection to the —C(O)O(CR²R³)— fragments of the compound represented by structural formula (I),
or T is a moiety represented by structural formula (IIf), wherein T is coupled to each —C(O)OCR²R³— fragment of formula (I) independently via two of W¹, W², A, B, or L² 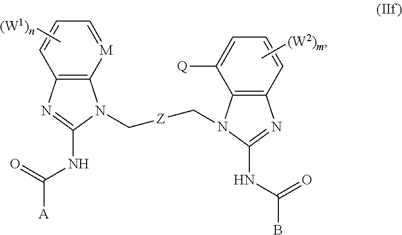
wherein:
M is N, C(X^{a}R^{a}), or C(X^{b}L¹L²-),
Q is —X^{a}R^{a} or -X^{b}L¹L²-,
each instance of W¹ and W² is independently selected from alkyl, amino, amido, carboxylic acid, ester, and hydrazido,
n and m are each independently 0, 1, 2, or 3,
Z is selected from alkylene, alkenylene, and alkynylene,
A and B are each independently 5-membered heteroaryl,
X^{a} and X^{b} are each independently selected from CH₂, NH, O, and S,
each instance of R^{a} is independently selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino and —O(alkylene)guanidino,
each instance of L¹ is independently selected from alkylene, heteroalkylene, alkenylene, alkynylene, cycloalkylene, heterocyclylene, arylene, and heteroarylene,
each instance of L² is independently selected from a bond, a linker moiety coupled to the respective L¹ and comprising a nitrogen atom coupled to the respective —C(O)O(CR²R³)— fragment of the compound represented by structural formula (I), or a second linker.

24. The compound of claim 23, wherein T is a moiety of formula (IId).

25. The compound of claim 23, wherein T is a moiety of formula (IIe).

26. The compound of claim 25, wherein the compound is

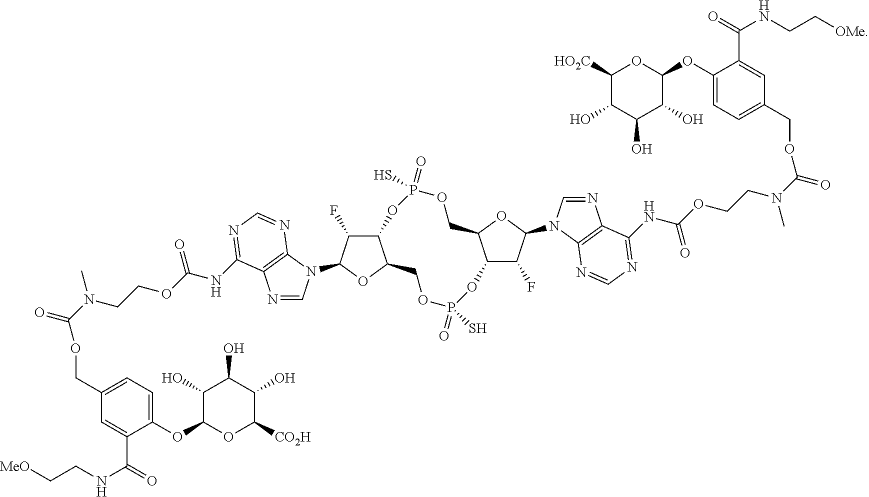 [SMILES: COCCNC(=O)c1cc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(NC(=O)OCCN(C)C(=O)OCc7ccc(O[C@@H]8O[C@H](C(=O)O)[C@@H](O)[C@H](O)[C@H]8O)c(C(=O)NCCOC)c7)ncnc65)O[C@@H]4CO[P@@](=O)(S)O[C@H]3[C@H]2F)ccc1O[C@@H]1O[C@H](C(=O)O)[C@@H](O)[C@H](O)[C@H]1O]

27. The compound of claim 23, wherein T is a moiety of formula (IIf).

28. The compound of claim 23, wherein T is coupled to each —C(O)O(CR²R³)— fragment via W¹ and W², and T is a moiety represented by formula (IIf1): 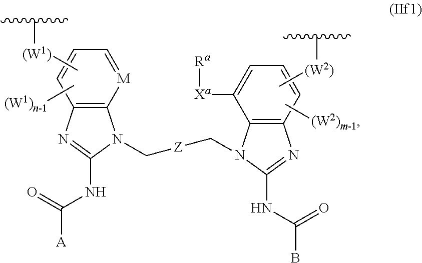
wherein each 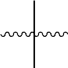
is a point of connection to the —C(O)O(CR²R³)— fragments of the compound represented by structural formula (I).

29. The compound of claim 23, wherein T is coupled to each —C(O)O(CR²R³)— fragment via A and B, and T is a moiety represented by formula (IIf2): 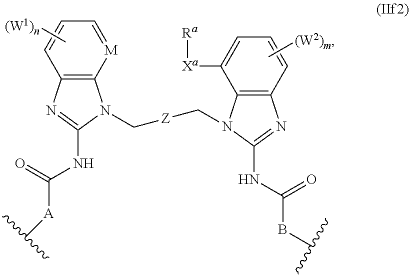
wherein each 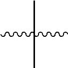
is a point of connection to the —C(O)O(CR²R³)— fragments of the compound represented by structural formula (I).

30. The compound of claim 23, wherein T is coupled to each —C(O)O(CR²R³)— fragment via two L², and T is a moiety represented by formula (IIf3): 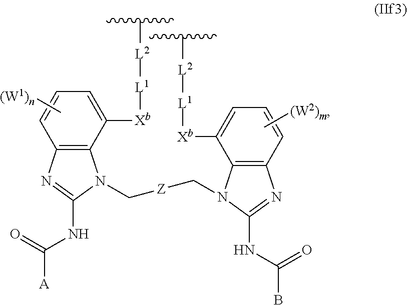
wherein each 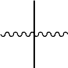
is a point of connection to the —C(O)O(CR²R³)— fragments of the compound represented by structural formula (I).

31. The compound of claim 30, wherein each L² is independently a second linker comprising ^{#}OC(O)NR⁵-L⁵-NR⁶, ^{#}OC(O)-L⁴-NR⁶, or ^{#}OC(O)NR⁵-L⁵-(heterocyclylene), wherein the heterocyclylene comprises a nitrogen atom connected to the respective —C(O)O(CR²R³)— fragment of the compound represented by structural formula (I), wherein # is the point of connection to the respective L¹,
each L⁵ is independently alkylene or aralkylene, and
each R⁵ and each R⁶ are each independently selected from H, alkyl, and dialkylaminoalkyl.

32. The compound of claim 1 or 2, wherein p is 1 and T is a moiety represented by formula (III): 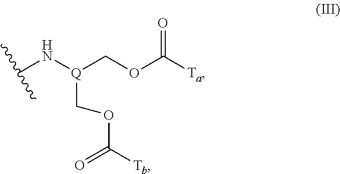
wherein Q is a branched linker moiety,
Tₐ and T_{b} are each independently a moiety comprising a Stimulator of Interferon Genes (STING) agonist, and 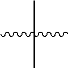
is a point of connection to the —C(O)O(CR²R³)— fragment of the compound represented by structural formula (III).

33. The compound of claim 32, wherein T is a moiety represented by formula (IIIa): 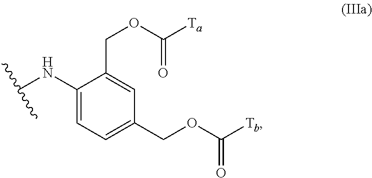
wherein: 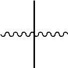
is the point of connection to the —C(O)O(CR²R³)— fragment of the compound represented by structural formula (I),
Tₐ and T_{b} are each independently a moiety represented by formula (IV): 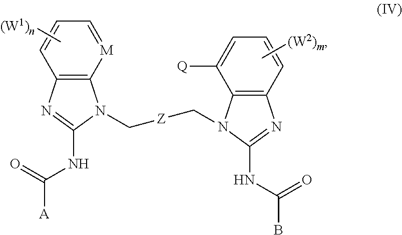
wherein:
Ta and Tb are each independently coupled to the —C(O)OCR²R³— fragment of formula (I) via W¹, W², A, B, or L²,
M is N, C(X^{a}R^{a}) or C(X^{b}L¹L²-),
Q is —X^{a}R^{a} or -X^{b}L¹L²-,
each instance of W¹ and W² is independently selected from alkyl, amino, amido, carboxylic acid, ester, and hydrazido, e.g. —C(O)NH-(alkyl)-N(alkyl)-;
n and m are each independently 0, 1, 2, or 3,
Z is selected from alkylene, alkenylene, and alkynylene,
A and B are each independently aryl or heteroaryl,
X^{a} and X^{b} are each independently selected from CH₂, NH, O, and S,
R^{a} is selected from H, alkyl, alkenyl, alkynyl, heteroalkyl, cycloalkyl, heterocyclyl, aryl, heteroaryl, heterocyclyl, aralkyl, heteroaralkyl, heterocyclylalkyl, cycloalkylalkyl, -(alkylene)carboxylic acid, -(alkylene)guanidino, -(alkylene)NHC(O)CH₂ guanidino, -(alkylene)O(alkylene)guanidino, and —O(alkylene)guanidino,
L¹ is selected from alkylene, heteroalkylene, alkenylene, alkynylene, cycloalkylene, heterocyclylene, arylene, and heteroarylene,
L² is a bond, or a linker moiety coupled to L¹ and comprising a nitrogen atom coupled to the —C(O)O(CR²R³)— fragment of the compound represented by structural formula (IIIa) or a second linker.

34. The compound of any one of claims 4-7, 10-23, 27-31, and 33, wherein A and B are each independently substituted or unsubstituted pyrazole or substituted or unsubstituted oxazole.

35. The compound of claim 34, wherein A and B are each independently substituted pyrazole or substituted oxazole, wherein the pyrazole and oxazole are each substituted with two C₁₋₃ alkyls.

36. The compound of any one of claims 1-35, wherein each instance of the first linker is independently selected in each instance from *(alkylene)O(alkylene)**, *(heteroalkylene)**, *(alkylene)**, *(heteroaralkylene)**, *(heteroalkylene)(heterocyclylene)**, *CH₂CH₂C(O)NHCH**, *(CH₂CH₂O)ₜ—**, and *(alkylene)(heteroarylene)(CH₂CH₂O)ₜ**,
wherein * indicates the point of connection to the —C(O)NH— fragment of the compound represented by structural formula (I), ** indicates the point of connection to U, and t represents an integer from 1-15.

37. The compound of claim 36, wherein each instance of the first linker is independently selected from *(CH₂CH₂O)ₜCH₂**, *(CH₂CH₂O)₂CH₂CH₂N(CH₃)CH₂**, *(CH₂CH₂O)₂CH₂CH₂N(CH₃)**, *(CH₂CH₂O)ₜ**, *(CH₂CH₂O)ₜCH₂CH₂NH**, *CH₂CH₂**, *(CH₂CH₂O)ₜCH₂**, *(CH₂CH₂O)ₜCH₂CH₂ heterocyclylene**, *(CH₂CH₂O)ₜCH₂**, *(CH₂CH₂O)ₜCH₂CH₂**, and *(CH₂CH₂O)ₜNH**.

38. The compound of claim 36 or claim 37, wherein t represents an integer from 1-6.

39. The compound of claim 38, wherein t represents an integer from 1-3.

40. The compound of any one of claims 1-39, wherein each instance of U is H.

41. The compound of any one of claims 1-39, wherein each instance of U is independently selected from alkyl, amino, azido, acetylenyl, alkylamino, heterocyclyl, alkoxy, —COOH, —P(O)(OH)₂, -DBCO and —OH.

42. The compound of claim 41, wherein the heterocyclyl comprises at least one nitrogen which is the point of connection to L^{u}.

43. The compound of claim 1, wherein the compound is

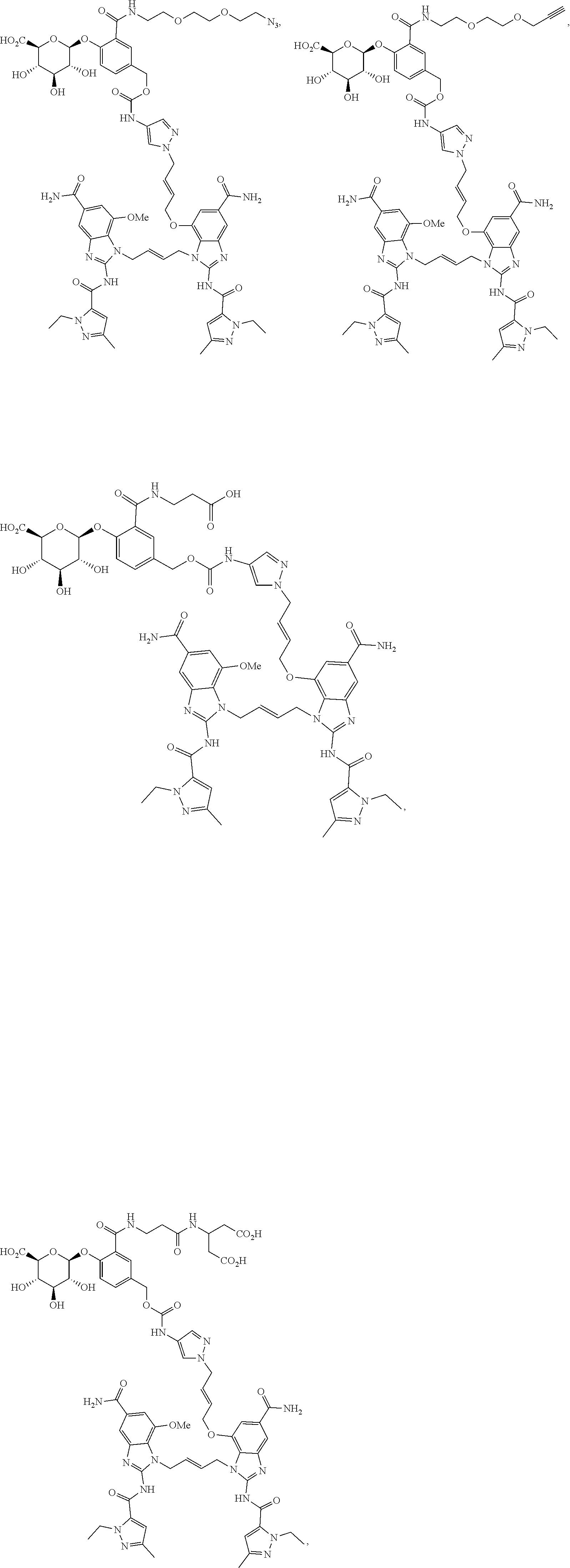

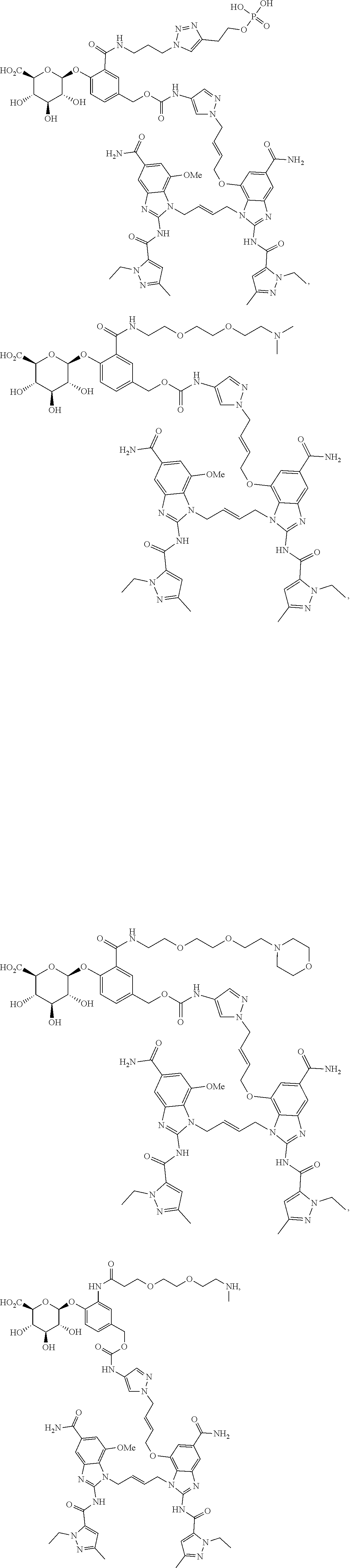

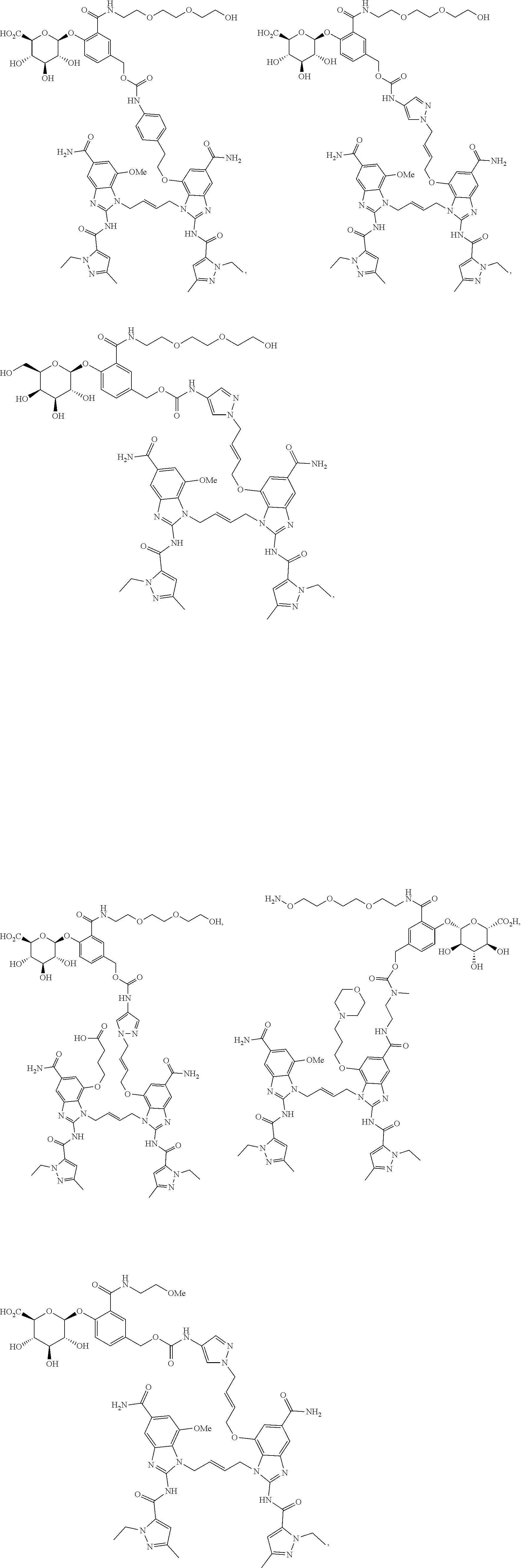

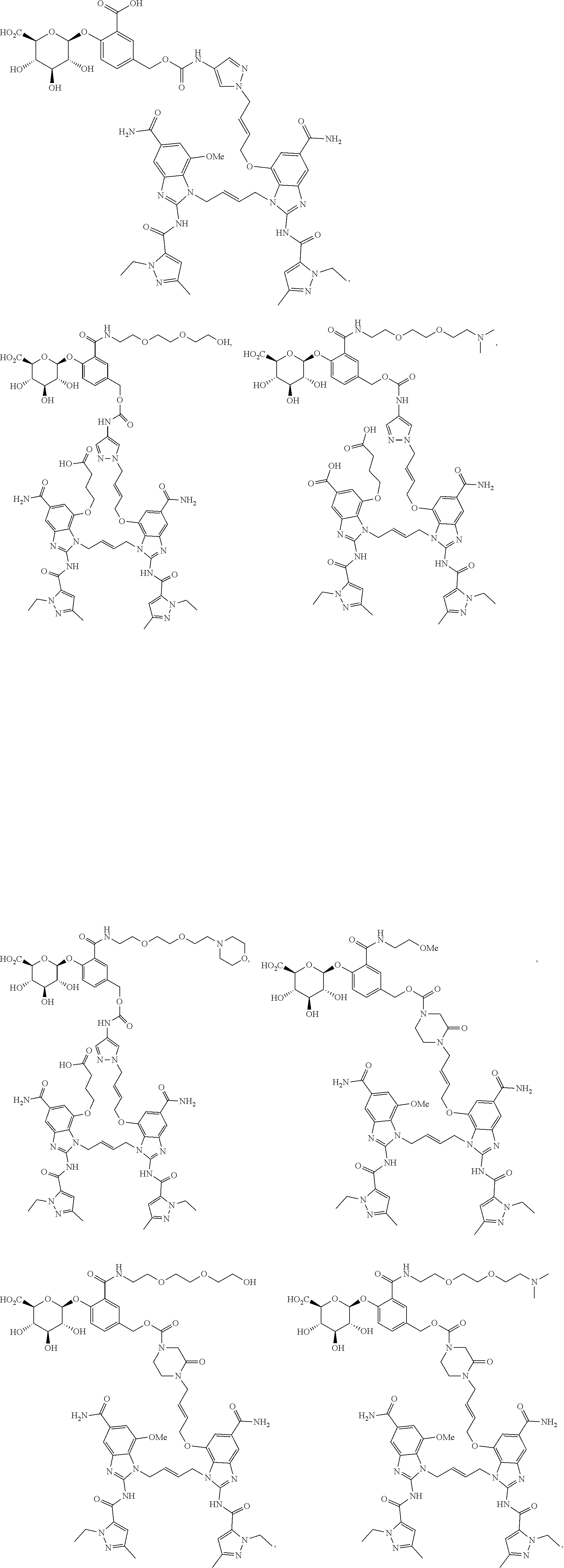

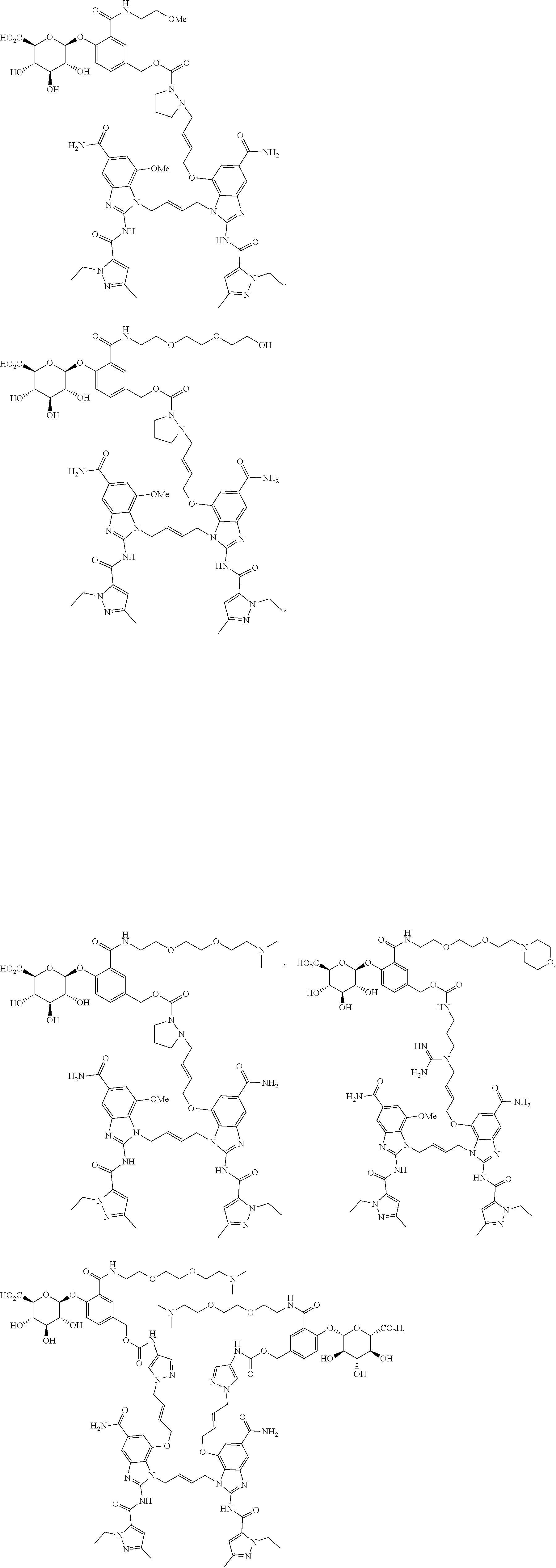

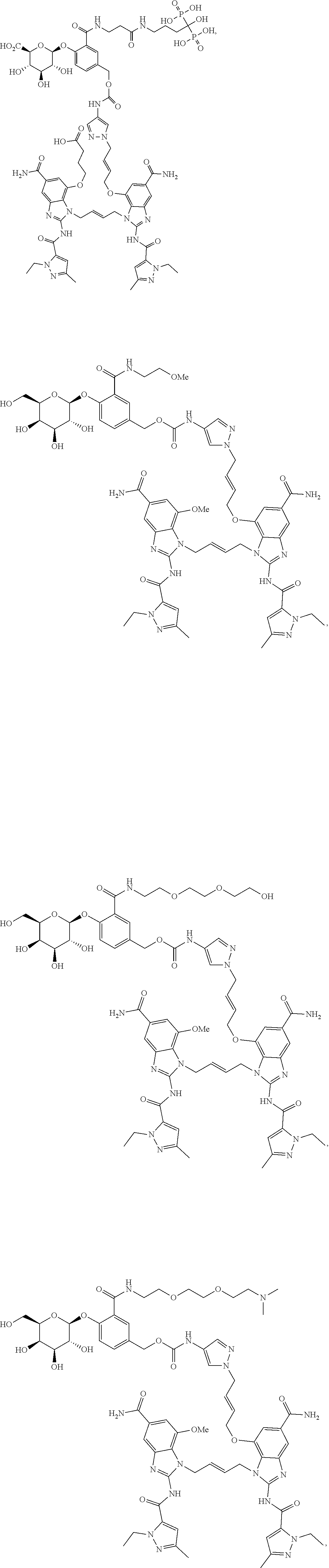

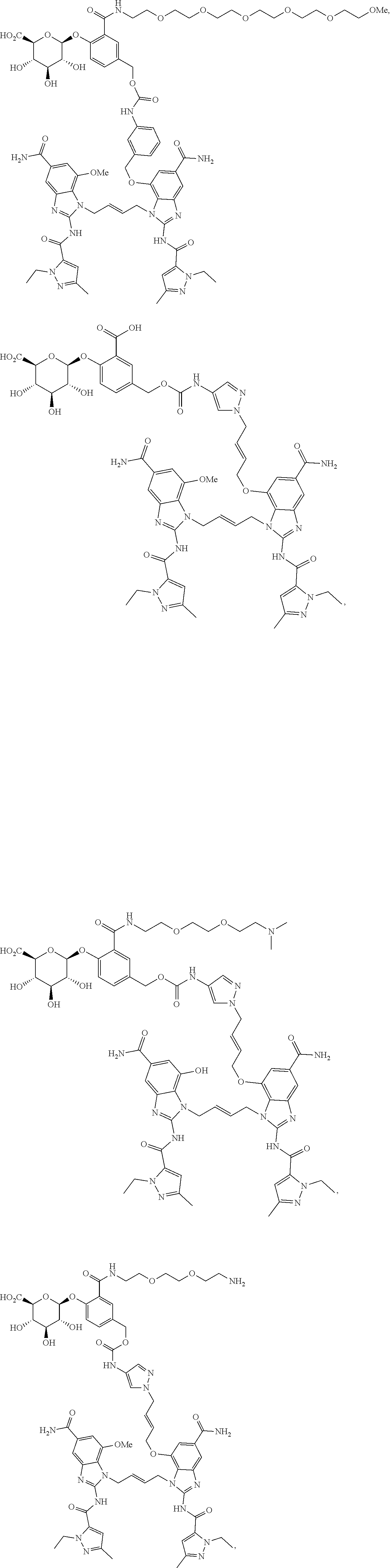

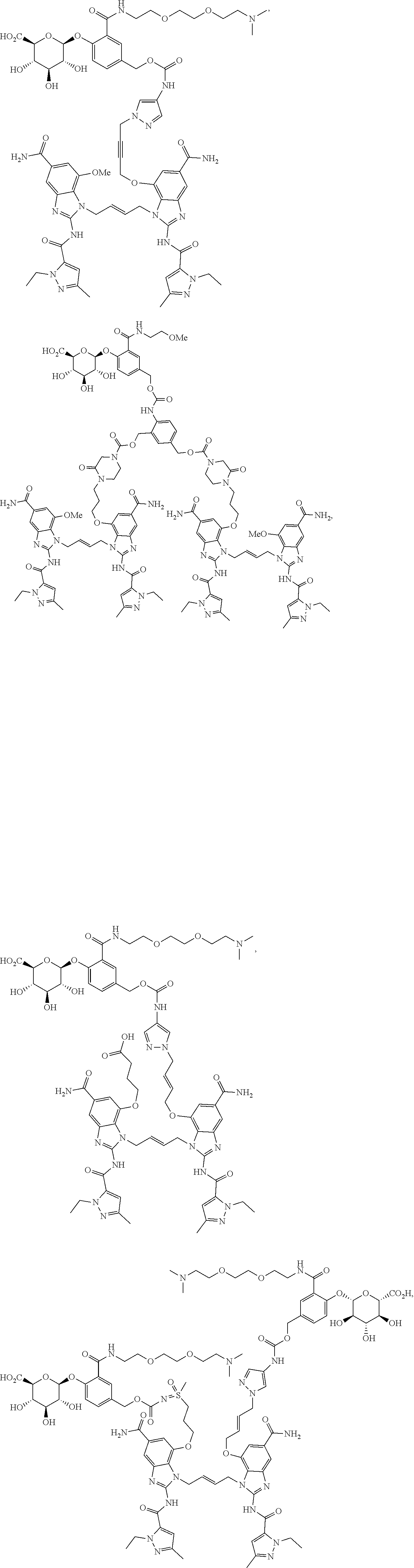

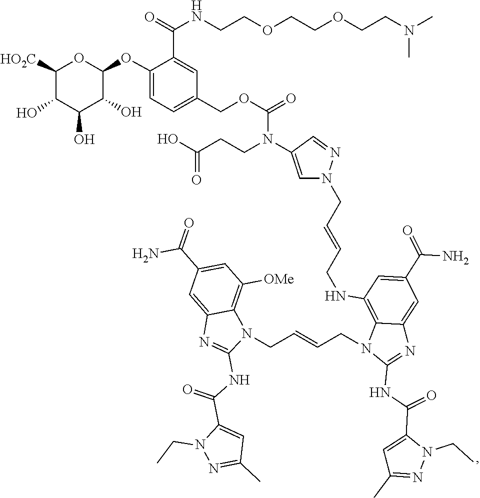 [SMILES: CCn1nc(C)cc1C(=O)Nc1nc2cc(C(N)=O)cc(NC/C=C/Cn3cc(N(CCC(=O)O)C(=O)OCc4ccc(O[C@@H]5O[C@H](C(=O)O)[C@@H](O)[C@H](O)[C@H]5O)c(C(=O)NCCOCCOCCN(C)C)c4)cn3)c2n1C/C=C/Cn1c(NC(=O)c2cc(C)nn2CC)nc2cc(C(N)=O)cc(OC)c21]

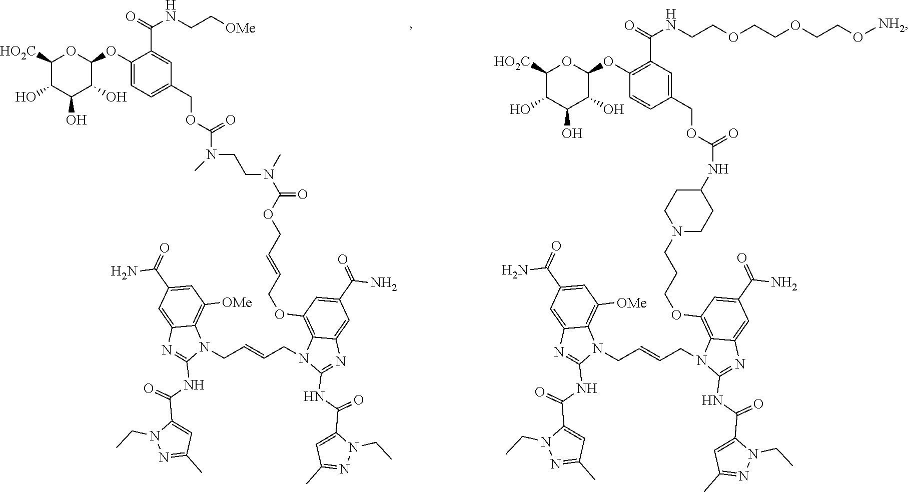 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/COC(=O)N(C)CCN(C)C(=O)OCc3ccc(O[C@@H]4O[C@H](C(=O)O)[C@@H](O)[C@H](O)[C@H]4O)c(C(=O)NCCOC)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c21]

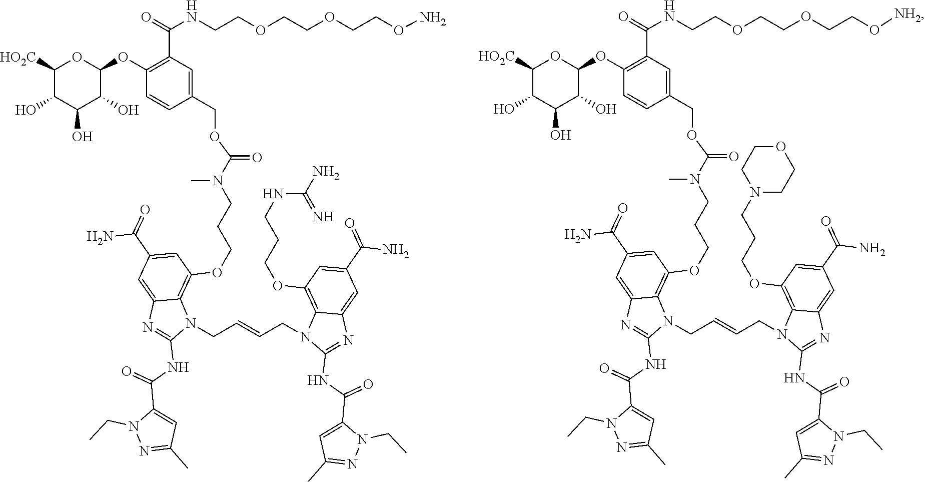 [SMILES: CCn1nc(C)cc1C(=O)Nc1nc2cc(C(N)=O)cc(OCCCNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21 ; CCn1nc(C)cc1C(=O)Nc1nc2cc(C(N)=O)cc(OCCCN3CCOCC3)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21]

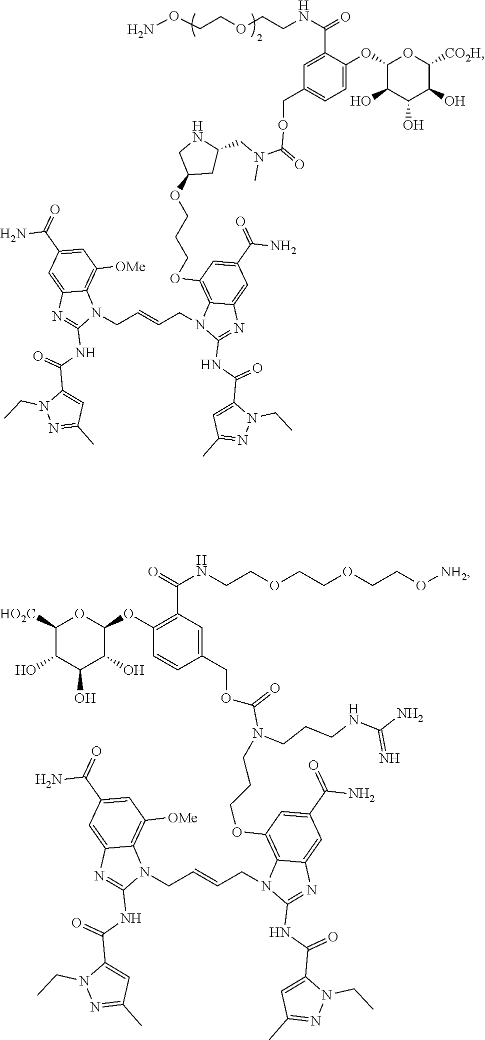

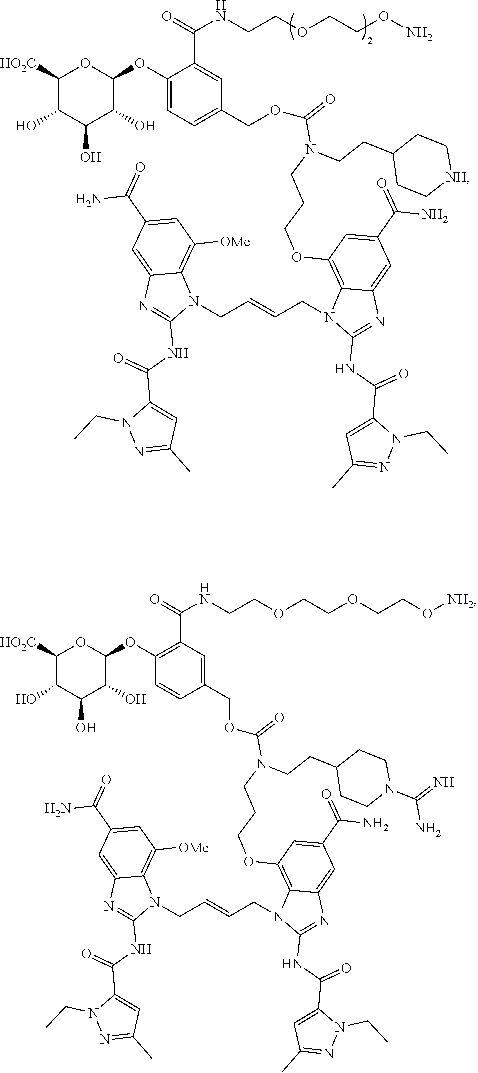

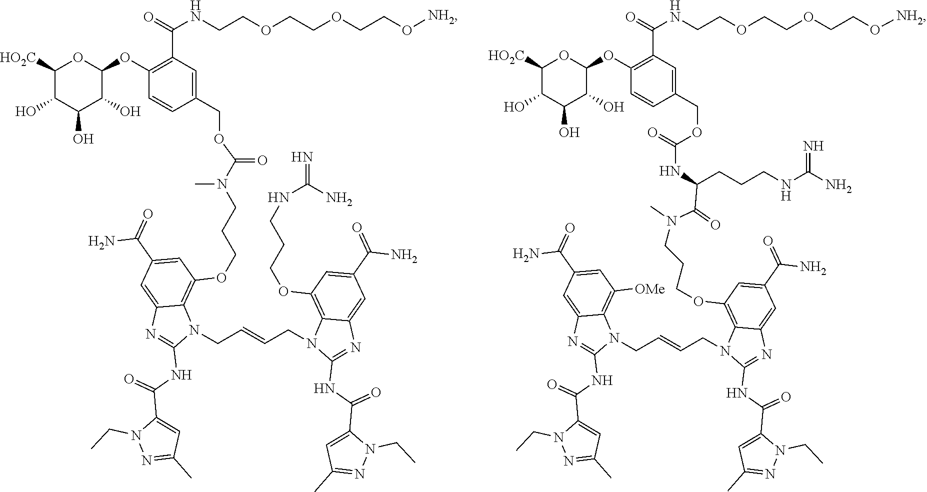 [SMILES: CCn1nc(C)cc1C(=O)Nc1nc2cc(C(N)=O)cc(OCCCNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CCCNC(=N)N)NC(=O)OCc3ccc(O[C@@H]4O[C@H](C(=O)O)[C@@H](O)[C@H](O)[C@H]4O)c(C(=O)NCCOCCOCCON)c3)c21]

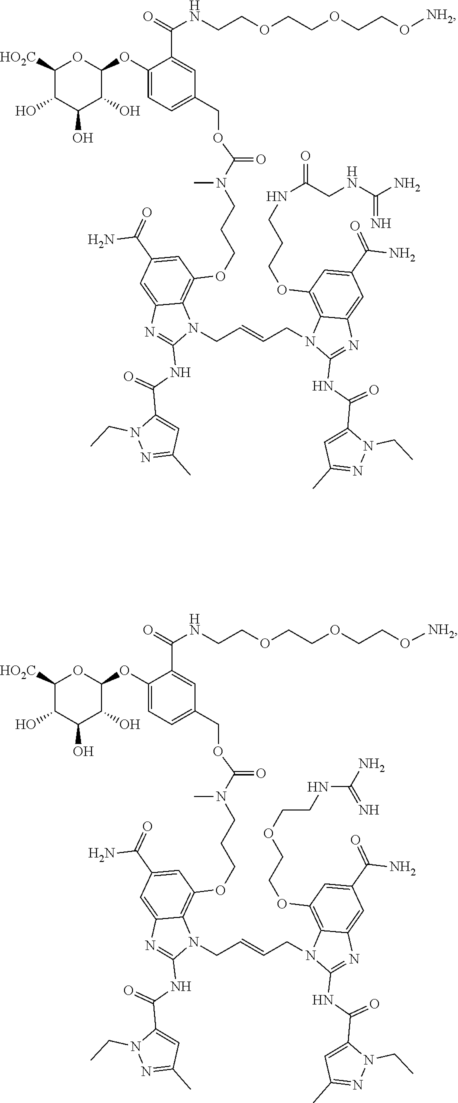 [SMILES: CCn1nc(C)cc1C(=O)Nc1nc2cc(C(N)=O)cc(OCCCNC(=O)CNC(=N)N)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c21 ; CCn1nc(C)cc1C(=O)Nc1nc2cc(C(N)=O)cc(OCCCN(C)C(=O)OCc3ccc(O[C@@H]4O[C@H](C(=O)O)[C@@H](O)[C@H](O)[C@H]4O)c(C(=O)NCCOCCOCCON)c3)c2n1C/C=C/Cn1c(NC(=O)c2cc(C)nn2CC)nc2cc(C(N)=O)cc(OCCOCCNC(=N)N)c21]

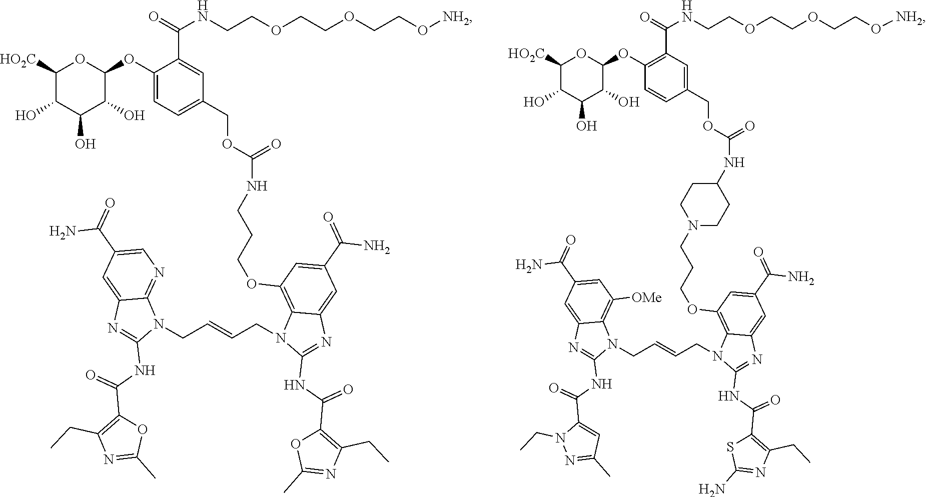 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNC(=O)OCc3ccc(O[C@@H]4O[C@H](C(=O)O)[C@@H](O)[C@H](O)[C@H]4O)c(C(=O)NCCOCCOCCON)c3)c21 ; CCc1nc(N)sc1C(=O)Nc1nc2cc(C(N)=O)cc(OCCCN3CCC(NC(=O)OCc4ccc(O[C@@H]5O[C@H](C(=O)O)[C@@H](O)[C@H](O)[C@H]5O)c(C(=O)NCCOCCOCCON)c4)CC3)c2n1C/C=C/Cn1c(NC(=O)c2cc(C)nn2CC)nc2cc(C(N)=O)cc(OC)c21]

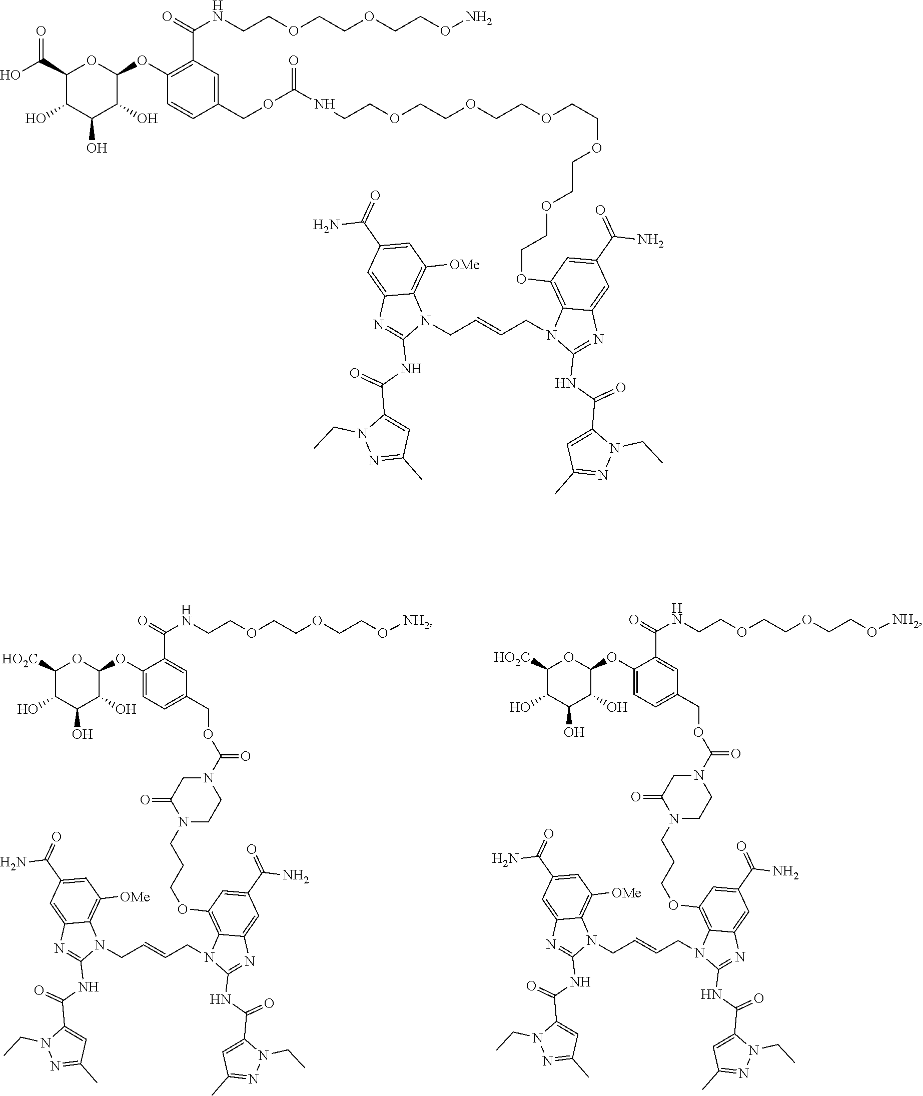

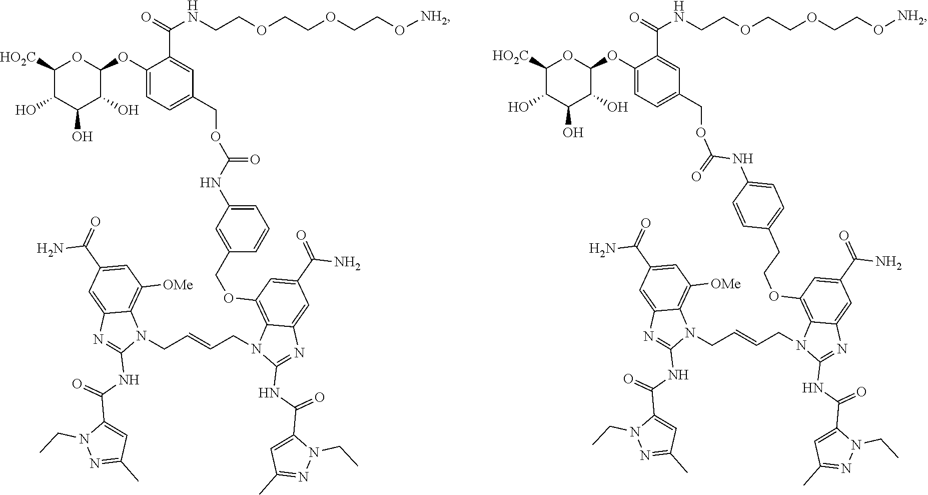 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCc3cccc(NC(=O)OCc4ccc(O[C@@H]5O[C@H](C(=O)O)[C@@H](O)[C@H](O)[C@H]5O)c(C(=O)NCCOCCOCCON)c4)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOCCOCCON)c4)cc3)c21]

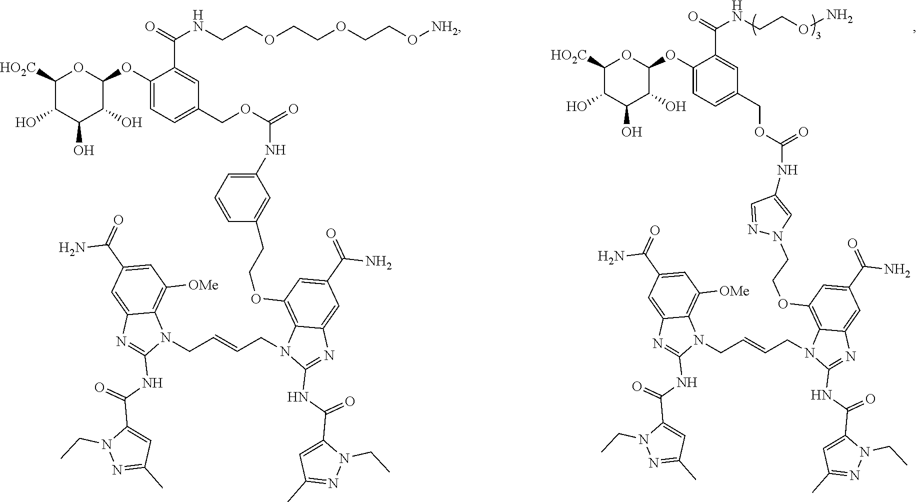

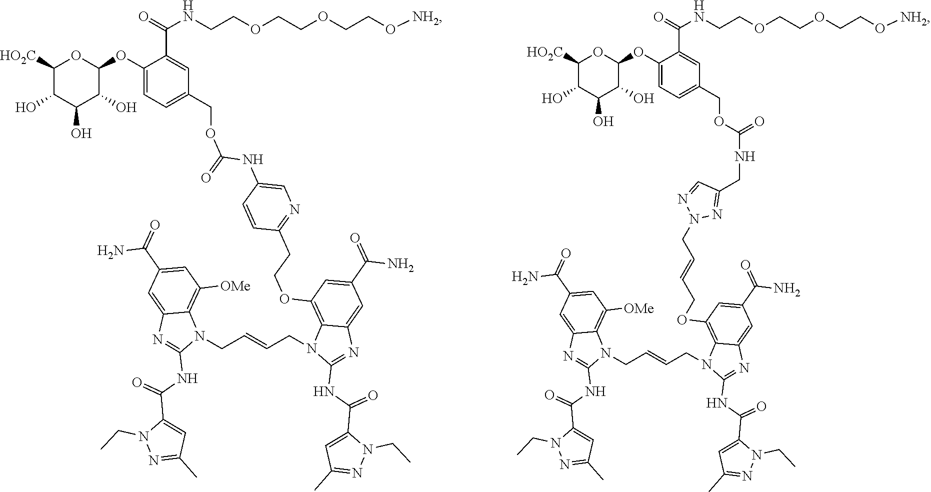 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCc3ccc(NC(=O)OCc4ccc(O[C@@H]5O[C@H](C(=O)O)[C@@H](O)[C@H](O)[C@H]5O)c(C(=O)NCCOCCOCCON)c4)cn3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3ncc(CNC(=O)OCc4ccc(O[C@@H]5O[C@H](C(=O)O)[C@@H](O)[C@H](O)[C@H]5O)c(C(=O)NCCOCCOCCON)c4)n3)c21]

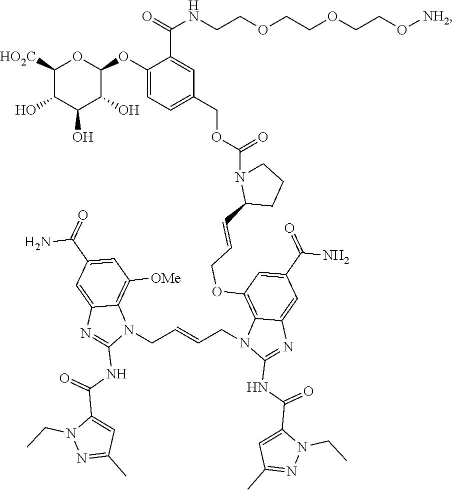 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/[C@@H]3CCCN3C(=O)OCc3ccc(O[C@@H]4O[C@H](C(=O)O)[C@@H](O)[C@H](O)[C@H]4O)c(C(=O)NCCOCCOCCON)c3)c21]

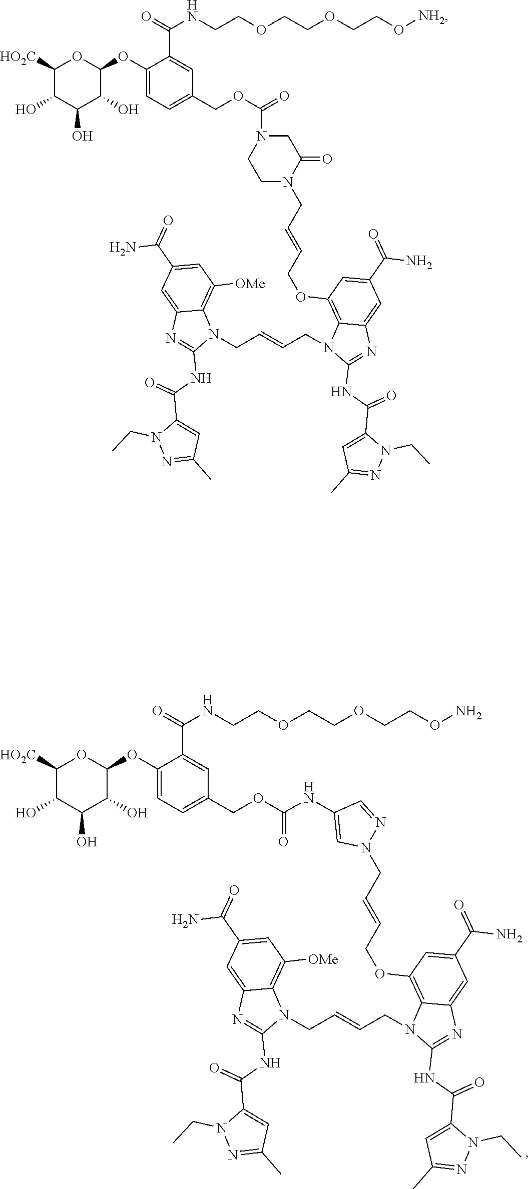 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCN(C(=O)OCc4ccc(O[C@@H]5O[C@H](C(=O)O)[C@@H](O)[C@H](O)[C@H]5O)c(C(=O)NCCOCCOCCON)c4)CC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)cn3)c21]

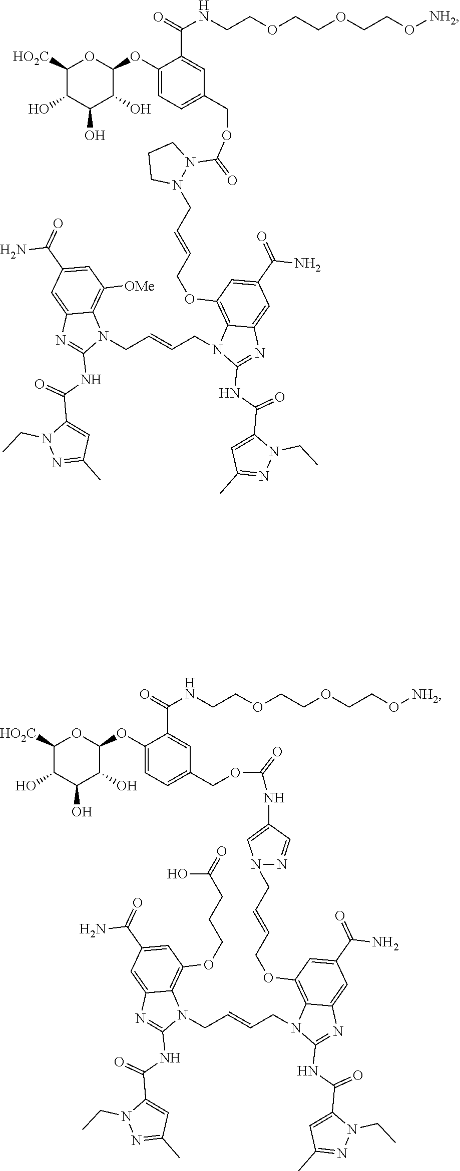 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/CN3CCCN3C(=O)OCc3ccc(O[C@@H]4O[C@H](C(=O)O)[C@@H](O)[C@H](O)[C@H]4O)c(C(=O)NCCOCCOCCON)c3)c21 ; CCn1nc(C)cc1C(=O)Nc1nc2cc(C(N)=O)cc(OC/C=C/Cn3cc(NC(=O)OCc4ccc(O[C@@H]5O[C@H](C(=O)O)[C@@H](O)[C@H](O)[C@H]5O)c(C(=O)NCCOCCOCCON)c4)cn3)c2n1C/C=C/Cn1c(NC(=O)c2cc(C)nn2CC)nc2cc(C(N)=O)cc(OCCCC(=O)O)c21]

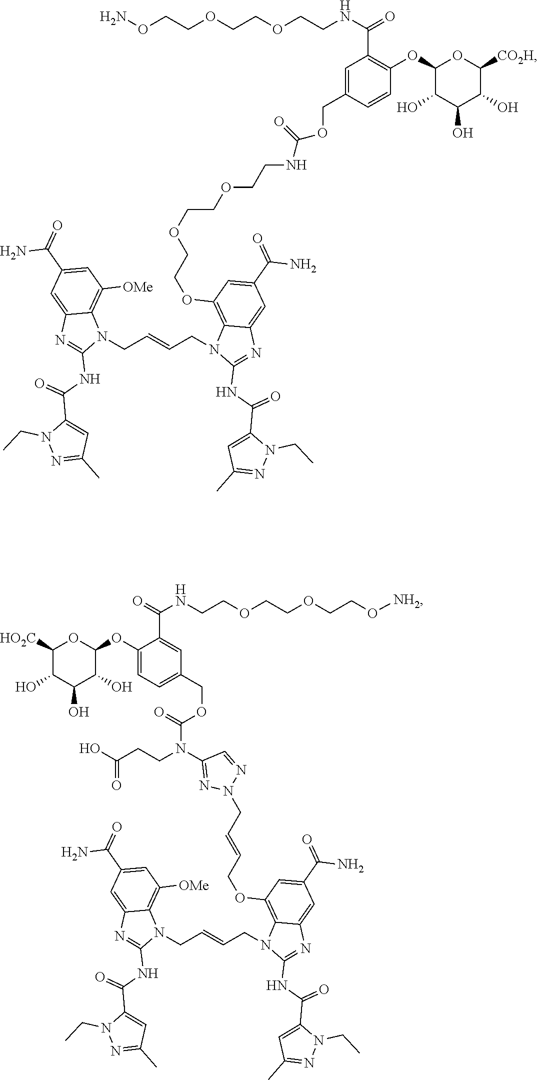 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCOCCOCCNC(=O)OCc3ccc(O[C@H]4O[C@@H](C(=O)O)[C@H](O)[C@@H](O)[C@@H]4O)c(C(=O)NCCOCCOCCON)c3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OC/C=C/Cn3ncc(N(CCC(=O)O)C(=O)OCc4ccc(O[C@@H]5O[C@H](C(=O)O)[C@@H](O)[C@H](O)[C@H]5O)c(C(=O)NCCOCCOCCON)c4)n3)c21]

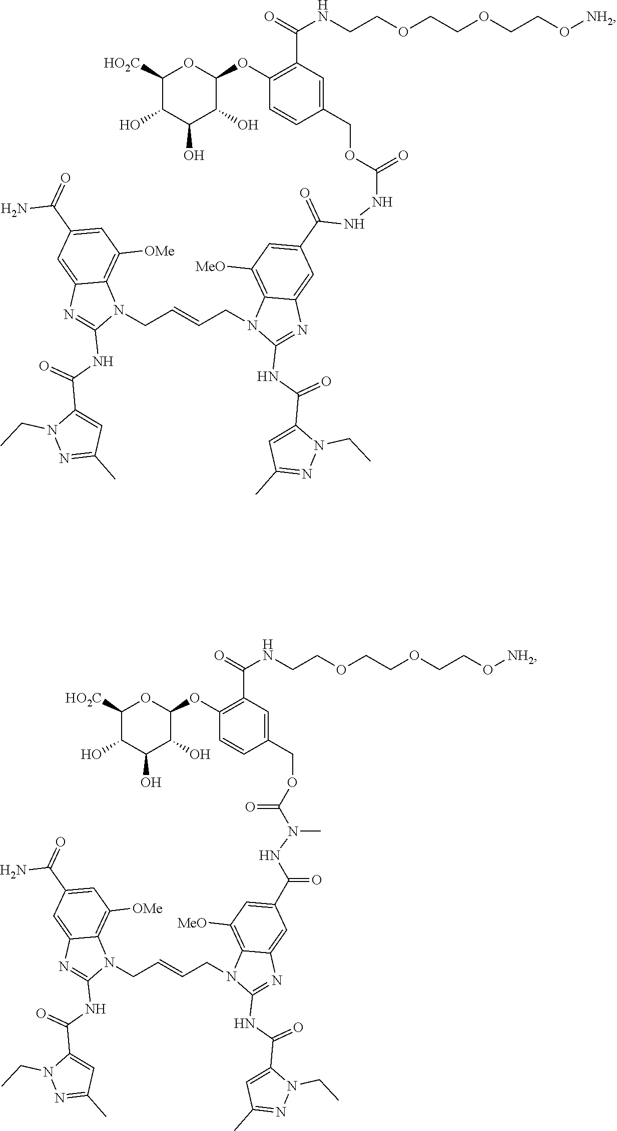 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NNC(=O)OCc3ccc(O[C@@H]4O[C@H](C(=O)O)[C@@H](O)[C@H](O)[C@H]4O)c(C(=O)NCCOCCOCCON)c3)cc(OC)c21 ; CCn1nc(C)cc1C(=O)Nc1nc2cc(C(N)=O)cc(OC)c2n1C/C=C/Cn1c(NC(=O)c2cc(C)nn2CC)nc2cc(C(=O)NN(C)C(=O)OCc3ccc(O[C@@H]4O[C@H](C(=O)O)[C@@H](O)[C@H](O)[C@H]4O)c(C(=O)NCCOCCOCCON)c3)cc(OC)c21]

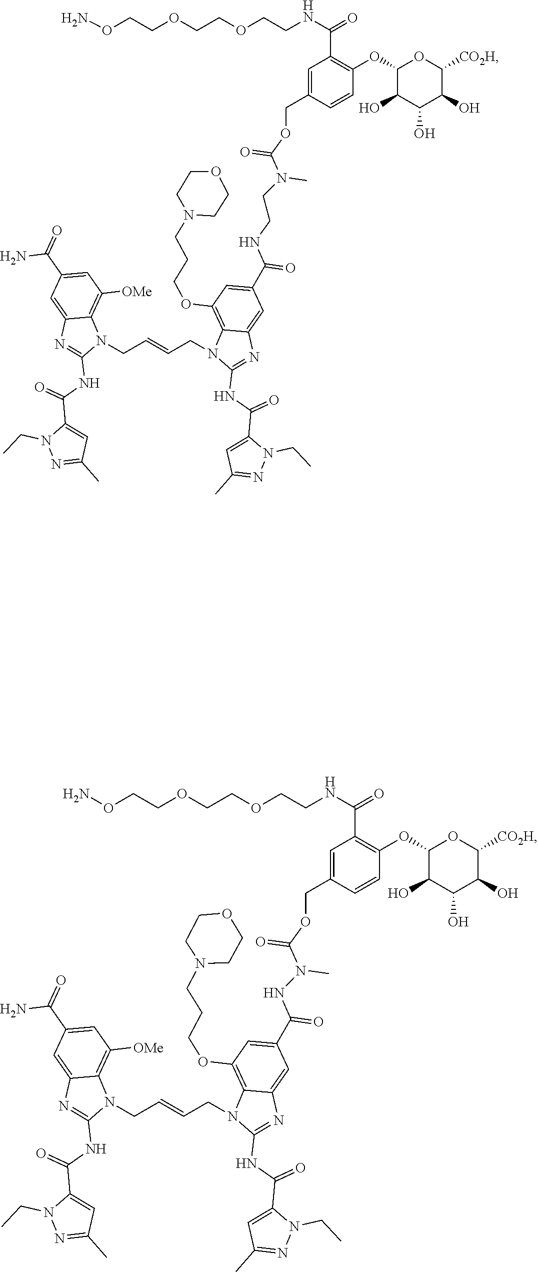 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NCCN(C)C(=O)OCc3ccc(O[C@@H]4O[C@H](C(=O)O)[C@@H](O)[C@H](O)[C@H]4O)c(C(=O)NCCOCCOCCON)c3)cc(OCCCN3CCOCC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(=O)NN(C)C(=O)OCc3ccc(O[C@@H]4O[C@H](C(=O)O)[C@@H](O)[C@H](O)[C@H]4O)c(C(=O)NCCOCCOCCON)c3)cc(OCCCN3CCOCC3)c21]

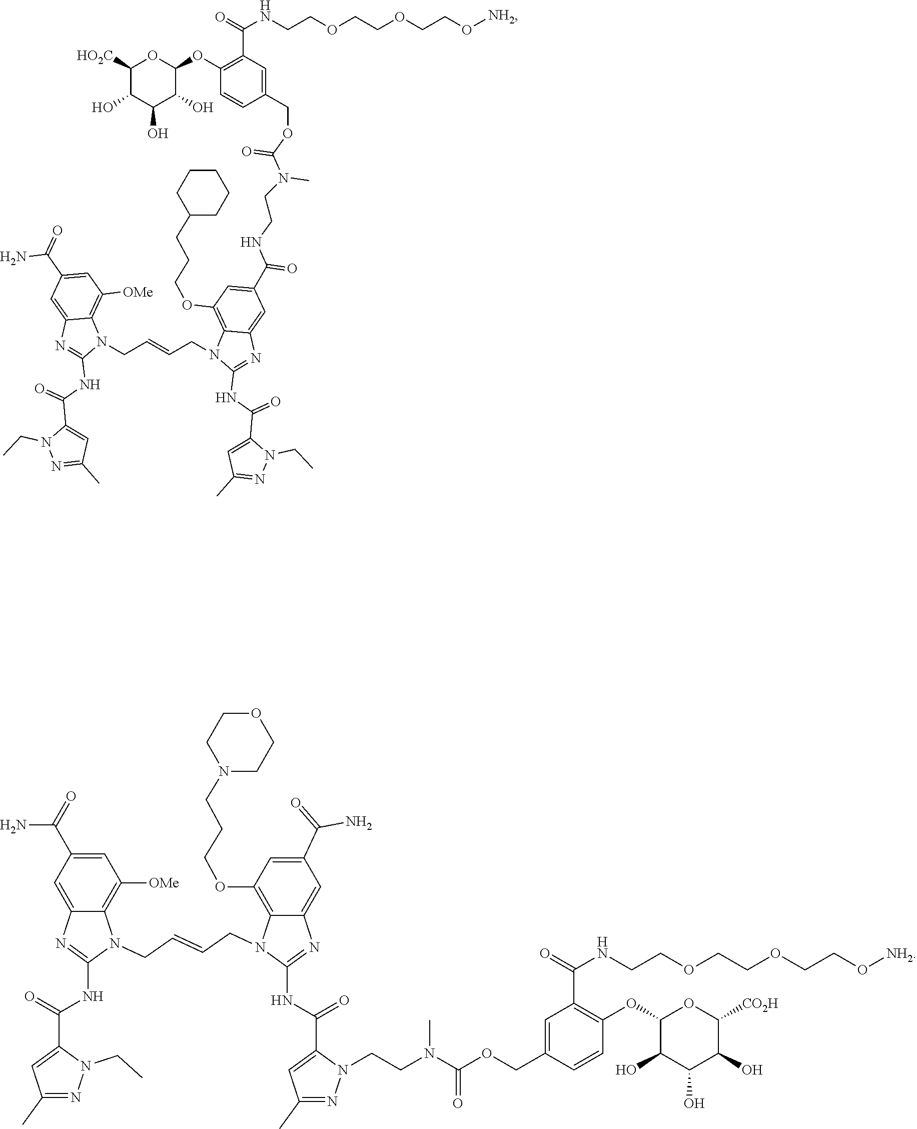 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NCCN(C)C(=O)OCc3ccc(O[C@@H]4O[C@H](C(=O)O)[C@@H](O)[C@H](O)[C@H]4O)c(C(=O)NCCOCCOCCON)c3)cc(OCCCC3CCCCC3)c21 ; CCn1nc(C)cc1C(=O)Nc1nc2cc(C(N)=O)cc(OC)c2n1C/C=C/Cn1c(NC(=O)c2cc(C)nn2CCN(C)C(=O)OCc2ccc(O[C@@H]3O[C@H](C(=O)O)[C@@H](O)[C@H](O)[C@H]3O)c(C(=O)NCCOCCOCCON)c2)nc2cc(C(N)=O)cc(OCCCN3CCOCC3)c21]

44. The compound of any one of claims 1-39, wherein each instance of U is a saccharide.

45. The compound of claim 44, wherein the saccharide is a glucuronide.

46. The compound of claim 45, wherein the glucuronide is

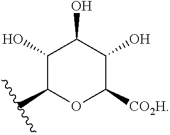 [SMILES: *[C@@H]1O[C@H](C(=O)O)[C@@H](O)[C@H](O)[C@H]1O]

47. The compound of claim 46, wherein the compound is selected from

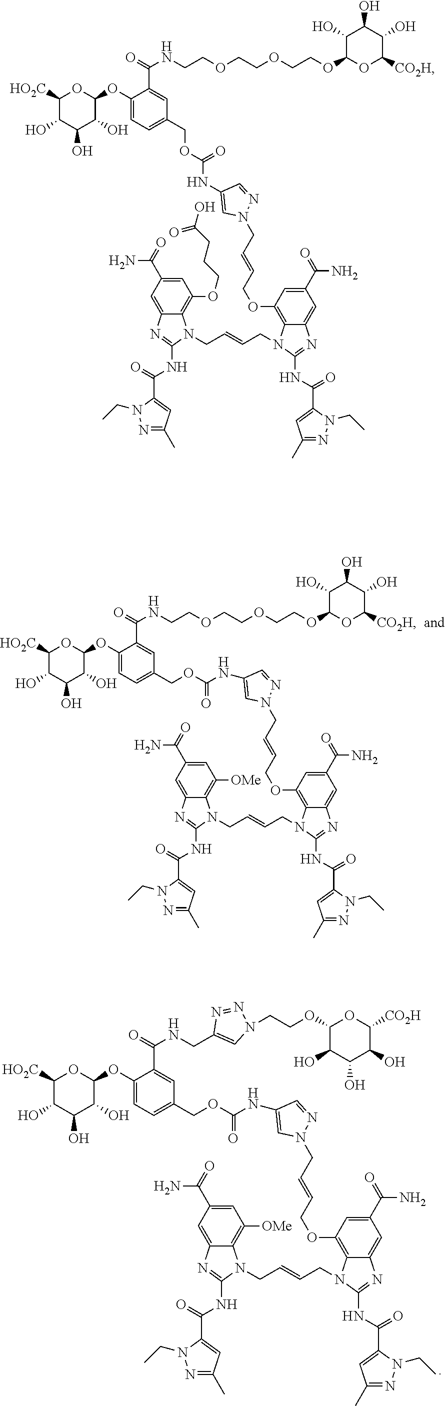

48. A pharmaceutical composition comprising a compound of any one of claims 1-47 and a pharmaceutically acceptable excipient.

49. A method of preventing or treating a disease mediated by stimulator of interferon genes (STING) in a subject in need thereof, comprising administering to the subject the compound of any one of claims 1-47 or the pharmaceutical composition of claim 48.

50. The method of claim 49, wherein the disease mediated by STING is cancer.

51. The method of claim 50, wherein the cancer is selected from lung cancer, small cell lung cancer, gastrointestinal cancer, colorectal cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi sarcoma, and melanoma.

52. A method of inducing an immune response in a subject in need thereof, comprising administering to the subject the compound of any one of claims 1-47 or the pharmaceutical composition of claim 48.

53. The method of claim 52, wherein the inducing of the immune response is effective to prevent or treat a disease mediated by STING in the subject.

54. The method of claim 53, wherein the disease mediated by STING is cancer.

55. The method of claim 54, wherein the cancer is selected from lung cancer, small cell lung cancer, gastrointestinal cancer, colorectal cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi sarcoma, and melanoma.

56. A method of modulating the activity of a STING adaptor protein comprising contacting the STING adaptor protein with the compound of any one of claims 1-47.

57. The method of claim 56, wherein the compound increases the activity of the STING adaptor protein.

58. The method of any one of claims 49-51, further comprising administering to the subject one or more additional therapeutic agents.

59. The method of any one of claims 52-55, further comprising administering to the subject one or more additional therapeutic agents.
